(19) United States
(12) Patent Application Publication
(10) Pub. No.: US 2018/0371021 A1
(43) Pub. Date: Dec. 27, 2018

(54) PEPTIDOMIMETIC MACROCYCLES AND USES THEREOF

(71) Applicant: Aileron Therapeutics, Inc., Cambridge, MA (US)

(72) Inventors: Manuel AIVADO, Chester Springs, PA (US); Vincent GUERLAVAIS, Arlington, MA (US); Karen OLSON, Waltham, MA (US)

(21) Appl. No.: 15/975,298

(22) Filed: May 9, 2018

Related U.S. Application Data
(60) Provisional application No. 62/650,527, filed on Mar. 30, 2018.
(60) Provisional application No. 62/571,881, filed on Oct. 13, 2017.
(60) Provisional application No. 62/504,922, filed on May 11, 2017.

(51) Int. Cl. C07K 7/64; A61P 35/00; A61K 47/64

(52) U.S. Cl. CPC C07K 7/64; A61P 35/00; A61K 31/519; A61K 2300/00; A61K 47/64

(57) ABSTRACT

The present disclosure describes the synthesis of peptidomimetic macrocycles and methods of using peptidomimetic macrocycles to treat a condition. The present disclosure also describes methods of using peptidomimetic macrocycles in combination with at least one additional pharmaceutically-active agent for the treatment of a condition, for example, cancer.

### CROSS REFERENCE

This application claims the benefit of U.S. Provisional Application No. 62/504,922, filed May 11, 2017; U.S. Provisional Application No. 62/571,881, filed Oct. 13, 2017; and U.S. Provisional Application No. 62/650,527, filed Mar. 30, 2018, each of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on May 31, 2018, is named 35224-823_201_SL.txt and is 1,195,677 bytes in size.

### BACKGROUND

The human transcription factor protein p53 induces cell cycle arrest and apoptosis in response to DNA damage and cellular stress, and thereby plays a critical role in protecting cells from malignant transformation. The E3 ubiquitin ligase MDM2, also known as HDM2, negatively regulates p53 function through a direct binding interaction, which neutralizes the p53 transactivation activity. Loss of p53 activity, either by deletion, mutation, or MDM2 overexpression, is the most common defect in human cancers.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE INVENTION

In some embodiments, the present disclosure provides a method of treating a condition in a subject in need thereof, the method comprising administering to the subject a therapeutically-effective amount of a peptidomimetic macrocycle and at least one pharmaceutically-active agent, wherein the peptidomimetic macrocycle and the at least one pharmaceutically-active agent are administered with a time separation of more than 61 minutes.

### BRIEF DESCRIPTION OF THE FIGURES

FIG. 1 shows that treatment with SP262 and SP154 resulted in decreased PD-L1 expression in HCT-116 p53^{+/+} cells, but not HCT-116 p53^{-/-} cells.

FIG. 2 illustrates the dosing regiments (DRs) used in the “3+3” dose escalation trial.

FIG. 3 shows drug concentration levels in patient plasma at all dose levels tested in Arm A (LEFT PANEL) and Arm B (RIGHT PANEL).

FIG. 4 shows fold-increase levels from baseline levels of plasma MIC-1 on cycle one, day one, two, or three (C1D1, C1D2, C1D3) at dose levels at or above 0.83 mg/kg.

FIG. 5 shows a waterfall plot that illustrates the anti-tumor activity of AP1 in patients of the Phase 1 dose-escalation trial.

FIG. 6 shows results of the anti-tumor activity study for 33 patients.

FIG. 7 shows the time-on-drug for evaluable p53-WT patients who had CRs, PRs, and SDs when dosed with AP1 at ≥3.2 mg/kg/cycle.

FIG. 8 PANEL A shows a 50-year-old patient with peripheral T-Cell Lymphoma (PTCL). FIG. 8 PANEL B shows that the lymph node returned to its normal size and was no longer detected by the PET tracer as being cancerous after six cycles of AP1 treatment. FIG. 8 PANEL C shows images of a 73-year-old patient with Merkel Cell Carcinoma (MCC). FIG. 8 PANEL D shows that skin lesions diminished in size and left only mild scar tissue after one cycle of AP1 treatment.

FIG. 9 LEFT PANEL shows PET scans from the first patient enrolled in the Phase 2 study prior to treatment with AP1. FIG. 9 RIGHT PANEL shows PET scans from the first patient enrolled in the Phase 2 study after 2 cycles of treatment with AP1.

FIG. 10 TOP PANEL shows percentage of human CD45 engraftment in bone marrow for the vehicle, and treatment with 20 mg/kg AP1. FIG. 10 BOTTOM PANEL shows the percentage survival of mice upon treatment with the vehicle or administration of AP1.

FIG. 11 shows a graph of MCF-7 cell proliferation determined using a WST-1 assay measured at the indicated time points after different numbers of MCF-7 cells were grown at 37° C. for a 24 hour growth period.

FIG. 12 shows MCF-7 cell proliferation when the cells were treated with varying concentrations of ribociclib.

FIG. 13 shows MCF-7 cell proliferation when the cells were treated with AP1 or AP1 with varying concentrations of ribociclib.

FIG. 14 shows MCF-7 cell proliferation when the cells were treated with varying concentrations of AP1. MCF-7 cells were treated with ribociclib or a combination of ribociclib and AP1 at concentrations of 0.1 μM, 0.3 μM, and 1 μM.

FIG. 15 shows MCF-7 cell proliferation when the cells were treated with ribociclib or ribociclib with varying concentrations of AP1.

FIG. 16 shows a combination index plot of ribociclib in MCF-7 cells.

FIG. 17 shows MCF-7 cell proliferation when the cells were treated with varying concentrations of abemaciclib.

FIG. 18 shows MCF-7 cell proliferation when the cells were treated with AP1 or AP1 with varying concentrations of abemaciclib.

FIG. 19 shows MCF-7 cell proliferation when the cells were treated with varying concentrations of AP1.

FIG. 20 shows MCF-7 cell proliferation when the cells were treated with abemaciclib or abemaciclib with varying concentrations of AP1.

FIG. 21 shows cell proliferation of MCF-7 cells when the cells were treated with palbociclib alone.

FIG. 22 shows cell proliferation of MCF-7 cells when the cells were treated with AP1 alone.

FIG. 23 shows MCF-7 cell proliferation when the cells were treated simultaneously with a fixed amount of AP1 and varying amounts of palbociclib.

FIG. 24 shows MCF-7 cell proliferation when the cells were treated simultaneously with a fixed amount of palbociclib and varying amounts of AP1.

FIG. 25 shows MCF-7 cell proliferation when the cells were treated with varying concentrations of AP1 and palbociclib in different orders over a period of 72 h.

FIG. 26 shows MCF-7 cell proliferation when the cells were pre-treated with AP1 for 24 h and subsequently treated with varying concentrations of palbociclib; and when the cells were pre-treated with varying concentrations of palbociclib for 24 h and subsequently treated with a fixed amount of AP1.

FIG. 27 shows MCF-7 cell proliferation when the cells were pre-treated with varying concentrations of AP1 for 24 h and subsequently treated with fixed amounts of palbociclib; and when the cells were pre-treated with fixed amounts of palbociclib and subsequently treated with varying concentrations of AP1.

FIG. 28 shows MOLT-3 cell proliferation when the cells were treated with palbociclib alone.

FIG. 29 shows MOLT-3 cell proliferation when the cells were treated with AP1 alone.

FIG. 30 shows the combination index plot of the treatment of MCF-7 cells with AP1 and palbociclib using a WST-1 assay.

FIG. 31 shows the combination index plot of the treatment of MCF-7 cells with AP1 and palbociclib using CyQUANT.

FIG. 32 shows the effects of AP1, palbociclib, or combination treatment with AP1+palbociclib on the median tumor volumes in the SJSA-1 osteosarcoma xenograft model.

FIG. 33 shows the effects of AP1, palbociclib, or combination treatment with AP1+palbociclib on the median tumor volumes in the MCF-7.1 human breast carcinoma xenograft model.

FIG. 34 shows individual tumor volumes of mice treated with MCF-7.1 human breast carcinoma xenografts treated with the vehicle.

FIG. 35 PANEL A shows the individual tumor volumes of mice treated with AP1 20 mg/kg qwk×4. FIG. 35 PANEL B shows the individual tumor volumes of mice treated with palbociclib 75 mg/kg qd×22. FIG. 35 PANEL C shows the individual tumor volumes of mice treated with AP1, and treated with palbociclib 6 h after administration of AP1. FIG. 35 PANEL D shows the individual tumor volumes of mice treated with palbociclib, and treated with AP1 6 h after administration of AP1.

FIG. 36 shows the effects of AP1, palbociclib, or combination treatment with AP1+palbociclib on the median tumor volumes in the A549 xenograft model.

FIG. 37 PANEL A shows the effect of vehicle treatment on median tumor volumes in the A549 xenograft model. FIG. 37 PANEL B shows the effect of vehicle treatment on median tumor volumes in the A549 xenograft model.

FIG. 38 shows C32 cell proliferation when the cells were treated with trametinib alone or trametinib in combination with varying concentrations of AP1.

FIG. 39 shows the combination index plot of the treatment of C32 cells with AP1 and trametinib.

FIG. 40 shows C32 cell proliferation when the cells were treated with AP1 alone or AP1 with varying concentrations of trametinib.

FIG. 41 shows C32 cell proliferation when the cells were treated with varying concentrations of AP1 and varying concentrations of trametinib.

FIG. 42 shows MEL-JUSO cell proliferation when the cells were treated with AP1 alone or AP1 and varying concentrations of trametinib.

FIG. 43 shows MEL-JUSO cell proliferation when the cells were treated with no agent, AP1 alone, trametinib alone, or 0.03 μM AP1 and 1.0 nM trametinib.

FIG. 44 shows MEL-JUSO cell proliferation when the cells were treated with trametinib alone or trametinib with varying concentrations of AP1

FIG. 45 shows the combination index plot of the treatment of MEL-JUSO cells with AP1 and trametinib.

FIG. 46 shows A375 cell proliferation when the cells were treated with AP1 alone or AP1 in combination with varying concentrations of trametinib.

FIG. 47 shows A375 cell proliferation when the cells were treated with trametinib alone or trametinib in combination with varying concentrations of AP1.

FIG. 48 shows the combination index plot of the treatment of A375 melanoma cells with AP1 and trametinib.

FIG. 49 shows C32 cell proliferation when the cells were treated with varying concentrations of binimetinib and AP1.

FIG. 50 shows C32 cell proliferation when the cells were treated with AP1 alone or AP1 in combination with varying concentrations of binimetinib.

FIG. 51 shows C32 cell proliferation when the cells were treated with binimetinib alone or binimetinib in combination with varying concentrations of AP1.

FIG. 52 shows the combination index plot of the treatment of C32 cells with AP1 and binimetinib.

FIG. 53 shows MEL-JUSO cell proliferation when the cells were treated with AP1 alone or AP1 in combination with varying concentrations of binimetinib.

FIG. 54 shows MEL-JUSO cell proliferation when the cells were treated with AP1 alone or AP1 in combination with varying concentrations of binimetinib.

FIG. 55 shows MEL-JUSO cell proliferation when the cells were treated with binimetinib alone or binimetinib in combination with varying concentrations of AP1.

FIG. 56 shows the combination index plot of the treatment of MEL-JUSO cells with AP1 and binimetinib.

FIG. 57 shows C32 cell proliferation when the cells were treated with AP1 alone or AP1 in combination with varying combinations of pimasertib.

FIG. 58 shows C32 cell proliferation when the cells were treated with varying concentrations of AP1 and pimasertib.

FIG. 59 shows C32 cell proliferation when the cells were treated with pimasertib alone or pimasertib in combination with varying concentrations of AP1.

FIG. 60 shows the combination index plot of the treatment of C32 cells with AP1 and pimasertib.

FIG. 61 shows MEL-JUSO cell proliferation when the cells were treated with AP1 alone or AP1 in combination with varying concentrations of pimasertib.

FIG. 62 shows MEL-JUSO cell proliferation when the cells were treated with AP1 and pimasertib.

FIG. 63 shows MEL-JUSO cell proliferation when the cells were treated with pimasertib alone or pimasertib in combination with varying concentrations of AP1.

FIG. 64 shows the combination index plot of the treatment of MEL-JUSO cells with AP1 and pimasertib.

FIG. 65 shows C32 cell proliferation when the cells were treated with AP1 alone or AP1 in combination with varying combinations of selumetinib.

FIG. 66 shows C32 cell proliferation when the cells were treated with varying concentrations of AP1 and selumetinib.

FIG. 67 shows C32 cell proliferation when the cells were treated with selumetinib alone or selumetinib in combination with varying concentrations of AP1.

FIG. 68 shows the combination index plot of the treatment of C32 cells with AP1 and selumetinib.

FIG. 69 shows MEL-JUSO cell proliferation when the cells were treated with AP1 alone or AP1 in combination with varying concentrations of pimasertib.

FIG. 70 shows MEL-JUSO cell proliferation when the cells were treated with AP1 and pimasertib.

FIG. 71 shows MEL-JUSO cell proliferation when the cells were treated with pimasertib alone or pimasertib in combination with varying concentrations of AP1.

FIG. 72 shows the combination index plot of the treatment of MEL-JUSO cells with AP1 and pimasertib.

FIG. 73 shows combination treatment and dosing regimens used to study the effects of AP1 to treat AML.

FIG. 74 shows the results of treatment with AP1 or Paclitaxel on individual mouse tumor volume by day.

FIG. 75 shows the results of combination treatment with AP1+paclitaxel on individual mouse tumor volume by day.

FIG. 76 shows the results of treatment with AP1 or Paclitaxel on individual mouse tumor volume by day on a Log₁₀ axis to show growth.

FIG. 77 shows the results of combination treatment with AP1+paclitaxel on individual mouse tumor volume by day on a Log₁₀ axis to show growth.

FIG. 78 shows the results of treatment with AP1 or Paclitaxel on individual mouse tumor volume % change from baseline by day.

FIG. 79 shows the results of combination treatment with AP1+paclitaxel on individual mouse tumor volume % change from baseline by day.

FIG. 80 shows the results of treatment with AP1 or Paclitaxel on median tumor volume % change from baseline by day.

FIG. 81 shows the results of combination treatment with AP1+paclitaxel on median tumor volume % change from baseline by day.

FIG. 82 shows the results of treatment with AP1 or Paclitaxel on average (±1 StDev) tumor volume % change from baseline by day.

FIG. 83 shows the results of combination treatment with AP1+paclitaxel on average (±1 StDev) tumor volume % change from baseline by day.

FIG. 84 compares the results of treatment with AP1, paclitaxel, or combination treatment with AP1+paclitaxel on the average % change in tumor volume from baseline per day.

FIG. 85 compares the results of treatment with AP1, paclitaxel, or combination treatment with AP1+paclitaxel on the average % change in tumor volume from baseline per day.

FIG. 86 shows the effect of treatment with AP1, paclitaxel, or combination treatment with AP1+paclitaxel on individual tumor volume % change from baseline on Day 28 per study group.

FIG. 87 shows the effect of treatment with AP1, eribulin, or combination treatment with AP1+eribulin on the average % change of tumor volume.

FIG. 88 shows the effect of treatment with AP1, eribulin, or combination treatment with AP1+eribulin on individual tumor volume % change from baseline on Day 28

FIG. 89 shows changes in the normalized body weights of mice treated under various dosing regimens of AP1, Abraxane®, or combination treatment with AP1+Abraxane® over a period of 12 days in the MCF-7.1 human breast carcinoma xenograft model.

FIG. 90 shows changes in tumor volumes (mm³) of mice treated under various dosing regimens over a period of 12 days in the MCF-7.1 human breast carcinoma xenograft model.

FIG. 91 PANEL A shows the results of vehicle treatment on tumor volumes (mm³) of mice using a CloudmanS91 malignant melanoma model. FIG. 91 PANEL B shows the results of treatment with anti-PD-1 on tumor volumes (mm³) of mice using a CloudmanS91 malignant melanoma model. FIG. 91 PANEL C shows the effect of treatment with twice a week treatment of AP1 at 20 mg/kg on tumor volumes (mm³) of mice using a CloudmanS91 malignant melanoma model. FIG. 91 PANEL D shows the effect of combination treatment with twice a week treatment of AP1 at 20 mg/kg and anti-PD-1 on tumor volumes (mm³) of mice using a CloudmanS91 malignant melanoma model.

FIG. 92 PANEL A shows the results of vehicle treatment on tumor volumes (mm³) of mice using a CloudmanS91 malignant melanoma model. FIG. 92 PANEL B shows the results of treatment with anti-PD-L1 on tumor volumes (mm³) of mice using a CloudmanS91 malignant melanoma model. FIG. 92 PANEL C shows the effect of treatment with twice a week treatment of AP1 at 20 mg/kg on tumor volumes (mm³) of mice using a CloudmanS91 malignant melanoma model. FIG. 92 PANEL D shows the effect of combination treatment with twice a week treatment of AP1 at 20 mg/kg and anti-PD-L1 on tumor volumes (mm³) of mice using a CloudmanS91 malignant melanoma model.

FIG. 93 PANEL A shows the results of vehicle treatment on tumor volumes (mm³) of mice using the A20 murine lymphoma model. FIG. 93 PANEL B shows the results of treatment with anti-PD-1 on tumor volumes (mm³) of mice using the A20 murine lymphoma model. FIG. 93 PANEL C shows the effect of treatment with twice a week treatment of AP1 at 20 mg/kg on tumor volumes (mm³) of mice using the A20 murine lymphoma model. FIG. 93 PANEL D shows the effect of combination treatment with twice a week treatment of AP1 at 20 mg/kg and anti-PD-1 on tumor volumes (mm³) of mice using the A20 murine lymphoma model.

FIG. 94 PANEL A shows the results of vehicle treatment on tumor volumes (mm³) of mice using the A20 murine lymphoma model. FIG. 94 PANEL B shows the results of treatment with anti-PD-L1 on tumor volumes (mm³) of mice using the A20 murine lymphoma model. FIG. 94 PANEL C shows the effect of treatment with twice a week treatment of AP1 at 20 mg/kg on tumor volumes (mm³) of mice using the A20 murine lymphoma model. FIG. 94 PANEL D shows the effect of combination treatment with twice a week treatment of AP1 at 20 mg/kg and anti-PD-L1 on tumor volumes (mm³) of mice using the A20 murine lymphoma model.

FIG. 95 PANEL A shows the results of vehicle treatment on tumor volumes (mm³) of mice using the M38 syngeneic colon carcinoma model. FIG. 95 PANEL B shows the results of treatment with anti-PD-L1 on tumor volumes (mm³) of mice using the M38 syngeneic colon carcinoma model. FIG. 95 PANEL C shows the effect of treatment with twice a week treatment of AP1 at 20 mg/kg on tumor volumes (mm³) of mice using the M38 syngeneic colon carcinoma model. FIG. 95 PANEL D shows the effect of combination treatment with twice a week treatment of AP1 at 20 mg/kg and anti-PD-L1 on tumor volumes (mm³) of mice using the M38 syngeneic colon carcinoma model.

FIG. 96 PANEL A shows the results of vehicle treatment on tumor volumes (mm³) of mice using the M38 syngeneic colon carcinoma model. FIG. 96 PANEL B shows the results of treatment with anti-PD-L1 on tumor volumes (mm³) of mice using the M38 syngeneic colon carcinoma model. FIG. 96 PANEL C shows the effect of treatment with twice a week treatment of AP1 at 20 mg/kg on tumor volumes (mm³) of mice using the M38 syngeneic colon carcinoma model. FIG. 96 PANEL D shows the effect of combination treatment with twice a week treatment of AP1 at 20 mg/kg and anti-PD-L1 on tumor volumes (mm³) of mice using the M38 syngeneic colon carcinoma model.

FIG. 97 PANEL A shows the results of vehicle treatment on tumor volumes (mm³) of mice using the CT26 undifferentiated colon carcinoma cell line. FIG. 97 PANEL B shows the results of treatment with anti-CTLA-4 9H10 on tumor volumes (mm³) of mice using the CT26 undifferentiated colon carcinoma cell line. FIG. 97 PANEL C shows the effect of treatment with twice a week treatment of AP1 at 20 mg/kg on tumor volumes (mm³) of mice using the CT26 undifferentiated colon carcinoma cell line. FIG. 97 PANEL D shows the effect of combination treatment with twice a week treatment of AP1 at 20 mg/kg and anti-CTLA-4 on tumor volumes (mm³) of mice using the CT26 undifferentiated colon carcinoma cell line.

### DETAILED DESCRIPTION

The human transcription factor protein p53 induces cell cycle arrest and apoptosis in response to DNA damage and cellular stress, and thereby plays a critical role in protecting cells from malignant transformation. The E3 ubiquitin ligase MDM2, also known as HDM2, negatively regulates p53 function through a direct binding interaction that neutralizes the p53 transactivation activity. Neutralization of p53 transactivation activity leads to export from the nucleus of p53 protein, which targets p53 for degradation via the ubiquitylation-proteasomal pathway. Loss of p53 activity, either by deletion, mutation, or MDM2 overexpression, is the most common defect in human cancers. Tumors that express wild type p53 are vulnerable to pharmacologic agents that stabilize or increase the concentration of active p53.

MDMX (MDM4) is a negative regulator of p53, and there is significant structural homology between the p53 binding interfaces of MDM2 and MDMX. The p53-MDM2 and p53-MDMX protein-protein interactions are mediated by the same 15-residue alpha-helical transactivation domain of p53, which inserts into hydrophobic clefts on the surface of MDM2 and MDMX. Three residues within this domain of p53 (F19, W23, and L26) are essential for binding to MDM2 and MDMX.

Provided herein are p53-based peptidomimetic macrocycles that modulate an activity of p53 and p53-based peptidomimetic macrocycles that inhibit the interactions between p53 and MDM2 and/or p53 and MDMX proteins. Also provided herein are the use of p53-based peptidomimetic macrocycles and an additional therapeutic agent for the treatment of a condition. Further, provided herein are p53-based peptidomimetic macrocycles and additional therapeutic agents that can be used for treating diseases, for example, cancer and other hyperproliferative diseases.

### Definitions

As used herein, the term “macrocycle” refers to a molecule having a chemical structure including a ring or cycle formed by at least 9 covalently bonded atoms.

As used herein, the term “peptidomimetic macrocycle” or “crosslinked polypeptide” refers to a compound comprising a plurality of amino acid residues joined by a plurality of peptide bonds and at least one macrocycle-forming linker which forms a macrocycle between a first naturally-occurring or non-naturally-occurring amino acid residue (or analogue) and a second naturally-occurring or non-naturally-occurring amino acid residue (or analogue) within the same molecule. Peptidomimetic macrocycle include embodiments where the macrocycle-forming linker connects the α-carbon of the first amino acid residue (or analogue) to the α-carbon of the second amino acid residue (or analogue). The peptidomimetic macrocycles optionally include one or more non-peptide bonds between one or more amino acid residues and/or amino acid analogue residues, and optionally include one or more non-naturally-occurring amino acid residues or amino acid analogue residues in addition to any which form the macrocycle. A “corresponding uncrosslinked polypeptide” when referred to in the context of a peptidomimetic macrocycle is understood to relate to a polypeptide of the same length as the macrocycle and comprising the equivalent natural amino acids of the wild-type sequence corresponding to the macrocycle.

AP1 is an alpha helical hydrocarbon crosslinked polypeptide macrocycle with an amino acid sequence less than 20 amino acids long that is derived from the transactivation domain of wild type human p53 protein. AP1 contains a phenylalanine, a tryptophan and a leucine amino acid in the same positions relative to each other as in the transactivation domain of wild type human p53 protein. AP1 has a single cross link spanning amino acids in the i to the i+7 position of the amino acid sequence and has more than three amino acids between the i+7 position and the carboxyl terminus. AP1 binds to human MDM2 and MDM4 and has an observed mass of 950-975 m/e as measured by electrospray ionization-mass spectrometry.

As used herein, the term “stability” refers to the maintenance of a defined secondary structure in solution by a peptidomimetic macrocycle as measured by circular dichroism, NMR or another biophysical measure, or resistance to proteolytic degradation in vitro or in vivo. Non-limiting examples of secondary structures contemplated herein are α-helices, 3₁₀ helices, β-turns, and β-pleated sheets.

As used herein, the term “helical stability” refers to the maintenance of an α-helical structure by a peptidomimetic macrocycle as measured by circular dichroism or NMR. In some embodiments, a peptidomimetic macrocycle can exhibit at least a 1.25, 1.5, 1.75, or 2-fold increase in α-helicity as determined by circular dichroism compared to a corresponding uncrosslinked macrocycle.

The term “amino acid” refers to a molecule containing both an amino group and a carboxyl group. Suitable amino acids include, without limitation, both the D- and L-isomers of the naturally-occurring amino acids, as well as non-naturally-occurring amino acids prepared by organic synthesis or other metabolic routes. The term amino acid, as used herein, includes, without limitation, α-amino acids, natural amino acids, non-natural amino acids, and amino acid analogues.

The term “α-amino acid” refers to a molecule containing both an amino group and a carboxyl group bound to a carbon which is designated the α-carbon.

The term “β-amino acid” refers to a molecule containing both an amino group and a carboxyl group in a β configuration.

The term “naturally-occurring amino acid” refers to any one of the twenty amino acids commonly found in peptides synthesized in nature, and known by the one letter abbreviations A, R, N, C, D, Q, E, G, H, I, L, K, M, F, P, S, T, W, Y and V.

The following table shows a summary of the properties of natural amino acids:

| | 3- | 1- | Side- | Side-chain | |
|---|---|---|---|---|---|
| | Letter | Letter | chain | charge | Hydropathy |
| Amino Acid | Code | Code | Polarity | (pH 7.4) | Index |
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | polar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | Positive (10%) | −3.2 |
| | | | | Neutral (90%) | |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

“Hydrophobic amino acids” include small hydrophobic amino acids and large hydrophobic amino acids. “Small hydrophobic amino acids” are glycine, alanine, proline, and analogues thereof. “Large hydrophobic amino acids” are valine, leucine, isoleucine, phenylalanine, methionine, tryptophan, and analogues thereof. “Polar amino acids” are serine, threonine, asparagine, glutamine, cysteine, tyrosine, and analogues thereof. “Charged amino acids” are lysine, arginine, histidine, aspartate, glutamate, and analogues thereof.

The term “amino acid analogue” refers to a molecule which is structurally similar to an amino acid and which can be substituted for an amino acid in the formation of a peptidomimetic macrocycle. Amino acid analogues include, without limitation, β-amino acids and amino acids wherein the amino or carboxy group is substituted by a similarly reactive group (e.g., substitution of the primary amine with a secondary or tertiary amine, or substitution of the carboxy group with an ester).

The term “non-natural amino acid” refers to an amino acid which is not one of the twenty amino acids commonly found in peptides synthesized in nature, and known by the one letter abbreviations A, R, N, C, D, Q, E, G, H, I, L, K, M, F, P, S, T, W, Y and V. Non-natural amino acids or amino acid analogues include, without limitation, structures according to the following:

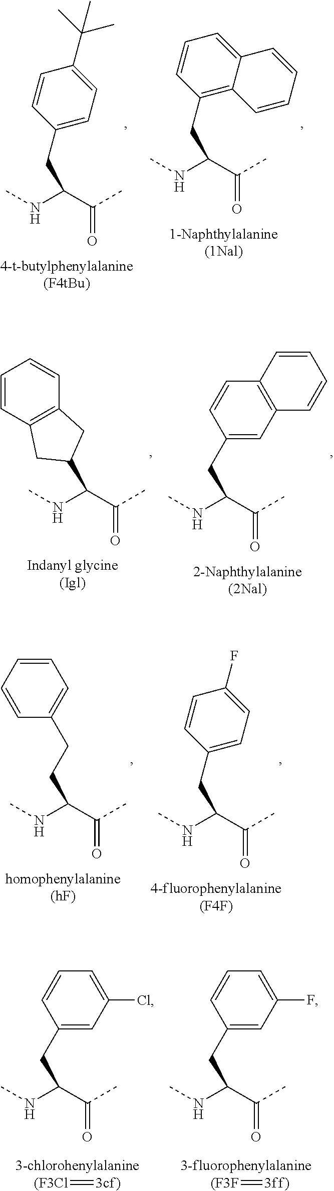

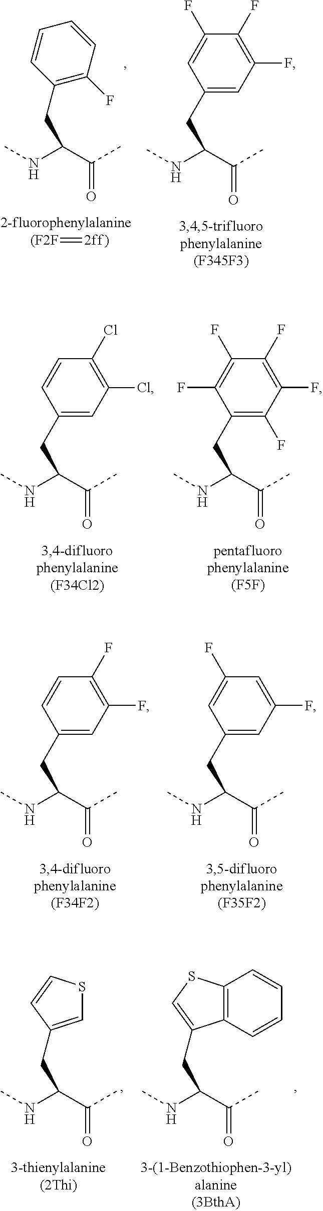

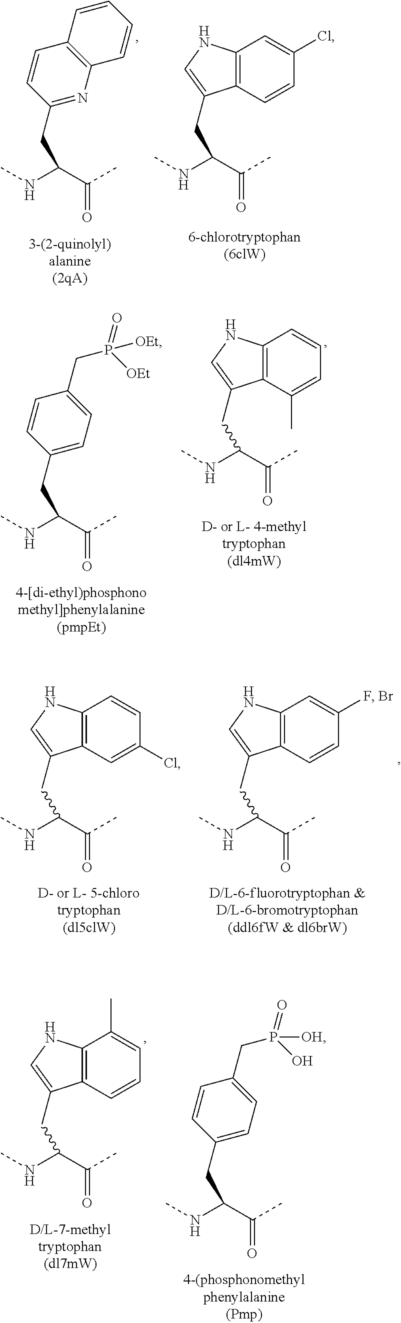

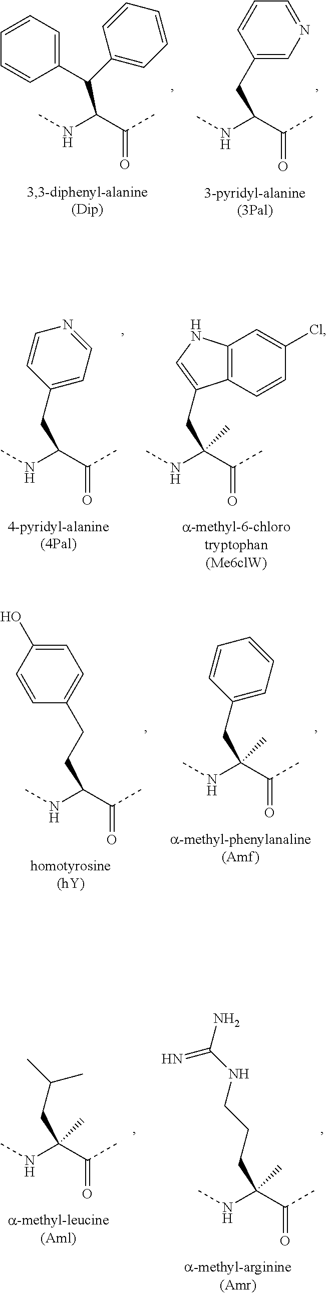

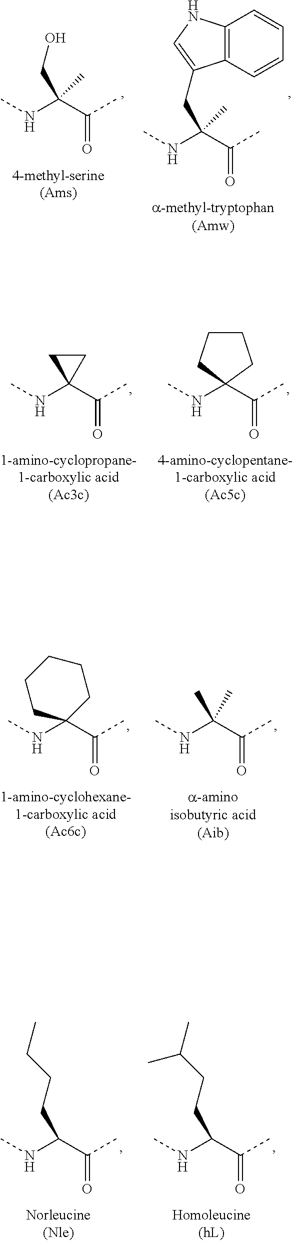

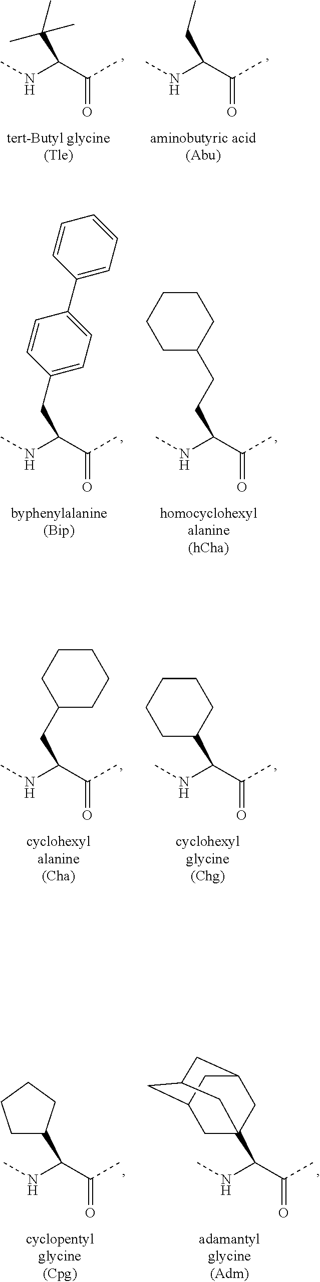

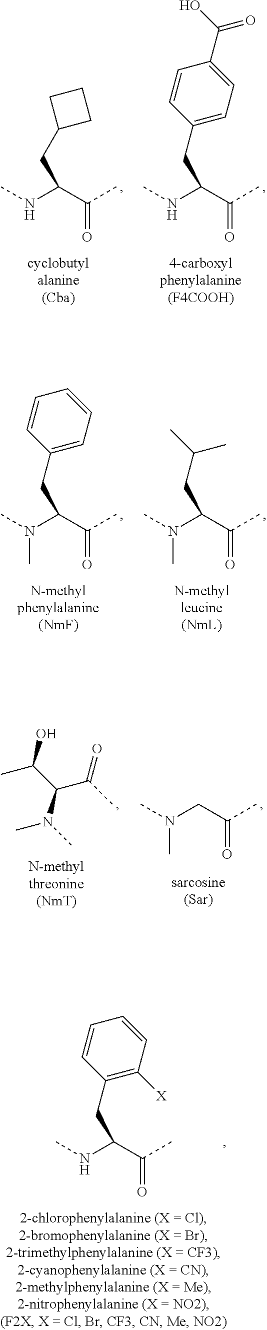

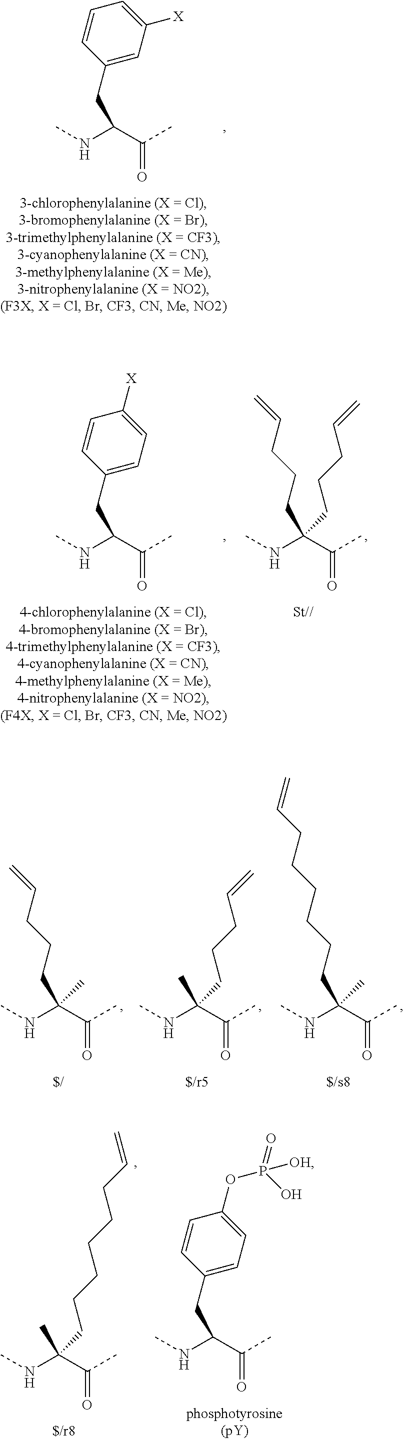

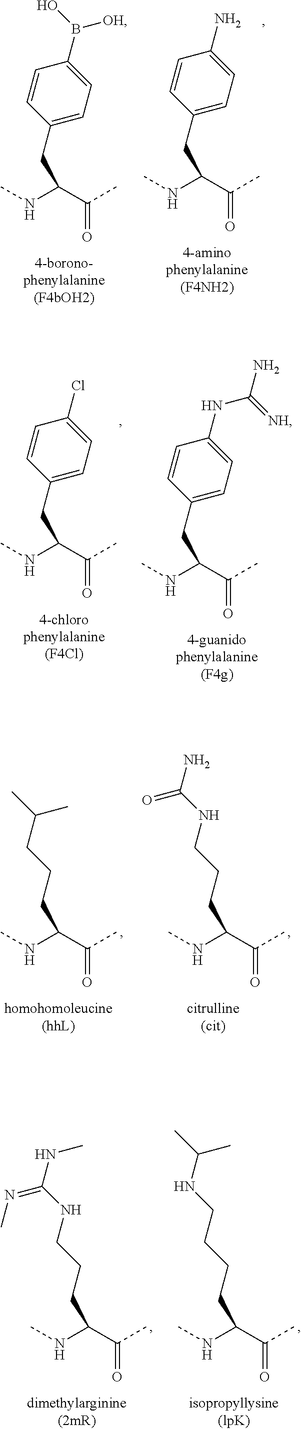

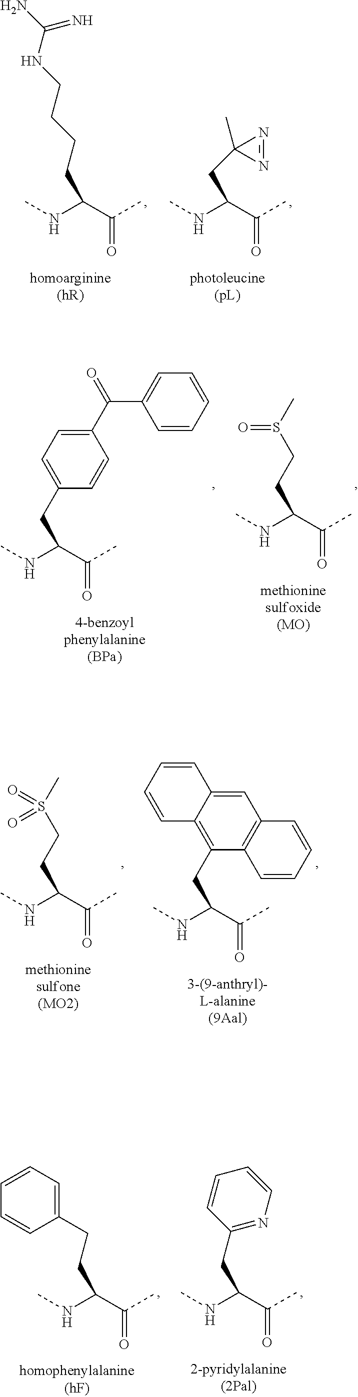

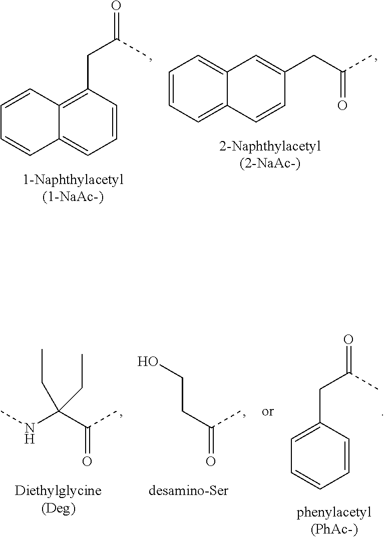

Amino acid analogues include β-amino acid analogues. Examples of β-amino acid analogues include, but are not limited to, the following: cyclic β-amino acid analogues; β-alanine; (R)-β-phenylalanine; (R)-1,2,3,4-tetrahydro-isoquinoline-3-acetic acid; (R)-3-amino-4-(1-naphthyl)-butyric acid; (R)-3-amino-4-(2,4-dichlorophenyl)butyric acid; (R)-3-amino-4-(2-chlorophenyl)-butyric acid; (R)-3-amino-4-(2-cyanophenyl)-butyric acid; (R)-3-amino-4-(2-fluorophenyl)-butyric acid; (R)-3-amino-4-(2-furyl)-butyric acid; (R)-3-amino-4-(2-methylphenyl)-butyric acid; (R)-3-amino-4-(2-naphthyl)-butyric acid; (R)-3-amino-4-(2-thienyl)-butyric acid; (R)-3-amino-4-(2-trifluoromethylphenyl)-butyric acid; (R)-3-amino-4-(3,4-dichlorophenyl)butyric acid; (R)-3-amino-4-(3,4-difluorophenyl)butyric acid; (R)-3-amino-4-(3-benzothienyl)-butyric acid; (R)-3-amino-4-(3-chlorophenyl)-butyric acid; (R)-3-amino-4-(3-cyanophenyl)-butyric acid; (R)-3-amino-4-(3-fluorophenyl)-butyric acid; (R)-3-amino-4-(3-methylphenyl)-butyric acid; (R)-3-amino-4-(3-pyridyl)-butyric acid; (R)-3-amino-4-(3-thienyl)-butyric acid; (R)-3-amino-4-(3-trifluoromethylphenyl)-butyric acid; (R)-3-amino-4-(4-bromophenyl)-butyric acid; (R)-3-amino-4-(4-chlorophenyl)-butyric acid; (R)-3-amino-4-(4-cyanophenyl)-butyric acid; (R)-3-amino-4-(4-fluorophenyl)-butyric acid; (R)-3-amino-4-(4-iodophenyl)-butyric acid; (R)-3-amino-4-(4-methylphenyl)-butyric acid; (R)-3-amino-4-(4-nitrophenyl)-butyric acid; (R)-3-amino-4-(4-pyridyl)-butyric acid; (R)-3-amino-4-(4-trifluoromethylphenyl)-butyric acid; (R)-3-amino-4-pentafluoro-phenylbutyric acid; (R)-3-amino-5-hexenoic acid; (R)-3-amino-5-hexynoic acid; (R)-3-amino-5-phenylpentanoic acid; (R)-3-amino-6-phenyl-5-hexenoic acid; (S)-1,2,3,4-tetrahydro-isoquinoline-3-acetic acid; (S)-3-amino-4-(1-naphthyl)-butyric acid; (S)-3-amino-4-(2,4-dichlorophenyl)butyric acid; (S)-3-amino-4-(2-chlorophenyl)-butyric acid; (S)-3-amino-4-(2-cyanophenyl)-butyric acid; (S)-3-amino-4-(2-fluorophenyl)-butyric acid; (S)-3-amino-4-(2-furyl)-butyric acid; (S)-3-amino-4-(2-methylphenyl)-butyric acid; (S)-3-amino-4-(2-naphthyl)-butyric acid; (S)-3-amino-4-(2-thienyl)-butyric acid; (S)-3-amino-4-(2-trifluoromethylphenyl)-butyric acid; (S)-3-amino-4-(3,4-dichlorophenyl)butyric acid; (S)-3-amino-4-(3,4-difluorophenyl)butyric acid; (S)-3-amino-4-(3-benzothienyl)-butyric acid; (S)-3-amino-4-(3-chlorophenyl)-butyric acid; (S)-3-amino-4-(3-cyanophenyl)-butyric acid; (S)-3-amino-4-(3-fluorophenyl)-butyric acid; (S)-3-amino-4-(3-methylphenyl)-butyric acid; (S)-3-amino-4-(3-pyridyl)-butyric acid; (S)-3-amino-4-(3-thienyl)-butyric acid; (S)-3-amino-4-(3-trifluoromethylphenyl)-butyric acid; (S)-3-amino-4-(4-bromophenyl)-butyric acid; (S)-3-amino-4-(4-chlorophenyl)-butyric acid; (S)-3-amino-4-(4-cyanophenyl)-butyric acid; (S)-3-amino-4-(4-fluorophenyl)-butyric acid; (S)-3-amino-4-(4-iodophenyl)-butyric acid; (S)-3-amino-4-(4-methylphenyl)-butyric acid; (S)-3-amino-4-(4-nitrophenyl)-butyric acid; (S)-3-amino-4-(4-pyridyl)-butyric acid; (S)-3-amino-4-(4-trifluoromethylphenyl)-butyric acid; (S)-3-amino-4-pentafluoro-phenylbutyric acid; (S)-3-amino-5-hexenoic acid; (S)-3-amino-5-hexynoic acid; (S)-3-amino-5-phenylpentanoic acid; (S)-3-amino-6-phenyl-5-hexenoic acid; 1,2,5,6-tetrahydropyridine-3-carboxylic acid; 1,2,5,6-tetrahydropyridine-4-carboxylic acid; 3-amino-3-(2-chlorophenyl)-propionic acid; 3-amino-3-(2-thienyl)-propionic acid; 3-amino-3-(3-bromophenyl)-propionic acid; 3-amino-3-(4-chlorophenyl)-propionic acid; 3-amino-3-(4-methoxyphenyl)-propionic acid; 3-amino-4,4,4-trifluoro-butyric acid; 3-aminoadipic acid; D-β-phenylalanine; β-leucine; L-β-homoalanine; L-β-homoaspartic acid γ-benzyl ester; L-β-homoglutamic acid δ-benzyl ester; L-β-homoisoleucine; L-β-homoleucine; L-β-homomethionine; L-β-homophenylalanine; L-β-homoproline; L-β-homotryptophan; L-β-homovaline; L-Nω-benzyloxycarbonyl-β-homolysine; Nω-L-β-homoarginine; O-benzyl-L-β-homohydroxyproline; O-benzyl-L-β-homoserine; O-benzyl-L-β-homothreonine; O-benzyl-L-β-homotyrosine; γ-trityl-L-β-homoasparagine; (R)-β-phenylalanine; L-β-homoaspartic acid γ-t-butyl ester; L-β-homoglutamic acid δ-t-butyl ester; L-Nω-β-homolysine; Nδ-trityl-L-β-homoglutamine; Nω-2,2,4,6,7-pentamethyl-dihydrobenzofuran-5-sulfonyl-L-β-homoarginine; O-t-butyl-L-β-homohydroxy-proline; O-t-butyl-L-β-homoserine; O-t-butyl-L-β-homothreonine; O-t-butyl-L-β-homotyrosine; 2-aminocyclopentane carboxylic acid; and 2-aminocyclohexane carboxylic acid.

Amino acid analogues include analogues of alanine, valine, glycine or leucine. Examples of amino acid analogues of alanine, valine, glycine, and leucine include, but are not limited to, the following: α-methoxyglycine; α-allyl-L-alanine; α-aminoisobutyric acid; α-methyl-leucine; β-(1-naphthyl)-D-alanine; β-(1-naphthyl)-L-alanine; β-(2-naphthyl)-D-alanine; β-(2-naphthyl)-L-alanine; β-(2-pyridyl)-D-alanine; β-(2-pyridyl)-L-alanine; β-(2-thienyl)-D-alanine; β-(2-thienyl)-L-alanine; β-(3-benzothienyl)-D-alanine; β-(3-benzothienyl)-L-alanine; β-(3-pyridyl)-D-alanine; β-(3-pyridyl)-L-alanine; β-(4-pyridyl)-D-alanine; β-(4-pyridyl)-L-alanine; β-chloro-L-alanine; β-cyano-L-alanine; β-cyclohexyl-D-alanine; β-cyclohexyl-L-alanine; β-cyclopenten-1-yl-alanine; β-cyclopentyl-alanine; β-cyclopropyl-L-Ala-OH.dicyclohexylammonium salt; β-t-butyl-D-alanine; β-t-butyl-L-alanine; γ-aminobutyric acid; L-α,β-diaminopropionic acid; 2,4-dinitro-phenylglycine; 2,5-dihydro-D-phenylglycine; 2-amino-4,4,4-trifluorobutyric acid; 2-fluoro-phenylglycine; 3-amino-4,4,4-trifluoro-butyric acid; 3-fluoro-valine; 4,4,4-trifluoro-valine; 4,5-dehydro-L-leu-OH.dicyclohexylammonium salt; 4-fluoro-D-phenylglycine; 4-fluoro-L-phenylglycine; 4-hydroxy-D-phenylglycine; 5,5,5-trifluoro-leucine; 6-aminohexanoic acid; cyclopentyl-D-Gly-OH.dicyclohexylammonium salt; cyclopentyl-Gly-OH.dicyclohexylammonium salt; D-α,β-diaminopropionic acid; D-α-aminobutyric acid; D-α-t-butylglycine; D-(2-thienyl)glycine; D-(3-thienyl)glycine; D-2-aminocaproic acid; D-2-indanylglycine; D-allylglycine.dicyclohexylammonium salt; D-cyclohexylglycine; D-norvaline; D-phenylglycine; β-aminobutyric acid; β-aminoisobutyric acid; (2-bromophenyl)glycine; (2-methoxyphenyl)glycine; (2-methylphenyl)glycine; (2-thiazoyl)glycine; (2-thienyl)glycine; 2-amino-3-(dimethylamino)-propionic acid; L-α,β-diaminopropionic acid; L-α-aminobutyric acid; L-α-t-butylglycine; L-(3-thienyl)glycine; L-2-amino-3-(dimethylamino)-propionic acid; L-2-aminocaproic acid dicyclohexyl-ammonium salt; L-2-indanylglycine; L-allylglycine.dicyclohexyl ammonium salt; L-cyclohexylglycine; L-phenylglycine; L-propargylglycine; L-norvaline; N-α-aminomethyl-L-alanine; D-α,γ-diaminobutyric acid; L-α,γ-diaminobutyric acid; β-cyclopropyl-L-alanine; (N-β-(2,4-dinitrophenyl))-L-α,β-diaminopropionic acid; (N-β-1-(4,4-dimethyl-2,6-dioxocyclohex-1-ylidene)ethyl)-D-α,β-diaminopropionic acid; (N-β-1-(4,4-dimethyl-2,6-dioxocyclohex-1-ylidene)ethyl)-L-α,β-diaminopropionic acid; (N-β-4-methyltrityl)-L-α,β-diaminopropionic acid; (N-3-allyloxycarbonyl)-L-α,β-diaminopropionic acid; (N-γ-1-(4,4-dimethyl-2,6-dioxocyclohex-1-ylidene)ethyl)-D-α,γ-diaminobutyric acid; (N-γ-1-(4,4-dimethyl-2,6-dioxocyclohex-1-ylidene)ethyl)-L-α,γ-diaminobutyric acid; (N-γ-4-methyltrityl)-D-α,γ-diaminobutyric acid; (N-γ-4-methyltrityl)-L-α,γ-diaminobutyric acid; (N-γ-allyloxycarbonyl)-L-α,γ-diaminobutyric acid; D-α,γ-diaminobutyric acid; 4,5-dehydro-L-leucine; cyclopentyl-D-Gly-OH; cyclopentyl-Gly-OH; D-allylglycine; D-homocyclohexylalanine; L-1-pyrenylalanine; L-2-aminocaproic acid; L-allylglycine; L-homocyclohexylalanine; and N-(2-hydroxy-4-methoxy-Bzl)-Gly-OH.

Amino acid analogues include analogues of arginine or lysine. Examples of amino acid analogues of arginine and lysine include, but are not limited to, the following: citrulline; L-2-amino-3-guanidinopropionic acid; L-2-amino-3-ureidopropionic acid; L-citrulline; Lys(Me)₂-OH; Lys(N₃)—OH; Nδ-benzyloxycarbonyl-L-ornithine; Nω-nitro-D-arginine; Nω-nitro-L-arginine; α-methyl-ornithine; 2,6-diaminoheptanedioic acid; L-ornithine; (Nδ-1-(4,4-dimethyl-2,6-dioxo-cyclohex-1-ylidene)ethyl)-D-ornithine; (Nδ-1-(4,4-dimethyl-2,6-dioxo-cyclohex-1-ylidene)ethyl)-L-ornithine; (Nδ-4-methyltrityl)-D-omithine; (Nδ-4-methyltrityl)-L-ornithine; D-omithine; L-omithine; Arg(Me)(Pbf)-OH; Arg(Me)₂-OH (asymmetrical); Arg(Me)₂-OH (symmetrical); Lys(ivDde)-OH; Lys(Me)₂-OH.HCl; Lys(Me₃)-OH chloride; Nω-nitro-D-arginine; and Nω-nitro-L-arginine.

Amino acid analogues include analogues of aspartic or glutamic acids. Examples of amino acid analogues of aspartic and glutamic acids include, but are not limited to, the following: α-methyl-D-aspartic acid; α-methyl-glutamic acid; α-methyl-L-aspartic acid; γ-methylene-glutamic acid; (N-γ-ethyl)-L-glutamine; [N-α-(4-aminobenzoyl)]-L-glutamic acid; 2,6-diaminopimelic acid; L-α-aminosuberic acid; D-2-aminoadipic acid; D-α-aminosuberic acid; α-aminopimelic acid; iminodiacetic acid; L-2-aminoadipic acid; threo-β-methyl-aspartic acid; γ-carboxy-D-glutamic acid γ,γ-di-t-butyl ester; γ-carboxy-L-glutamic acid γ,γ-di-t-butyl ester; Glu(OAll)-OH; L-Asu(OtBu)-OH; and pyroglutamic acid.

Amino acid analogues include analogues of cysteine and methionine. Examples of amino acid analogues of cysteine and methionine include, but are not limited to, Cys(farnesyl)-OH, Cys(farnesyl)-OMe, α-methyl-methionine, Cys(2-hydroxyethyl)-OH, Cys(3-aminopropyl)-OH, 2-amino-4-(ethylthio)butyric acid, buthionine, buthioninesulfoximine, ethionine, methionine methylsulfonium chloride, selenomethionine, cysteic acid, [2-(4-pyridyl)ethyl]-DL-penicillamine, [2-(4-pyridyl)ethyl]-L-cysteine, 4-methoxybenzyl-D-penicillamine, 4-methoxybenzyl-L-penicillamine, 4-methylbenzyl-D-penicillamine, 4-methylbenzyl-L-penicillamine, benzyl-D-cysteine, benzyl-L-cysteine, benzyl-DL-homocysteine, carbamoyl-L-cysteine, carboxyethyl-L-cysteine, carboxymethyl-L-cysteine, diphenylmethyl-L-cysteine, ethyl-L-cysteine, methyl-L-cysteine, t-butyl-D-cysteine, trityl-L-homocysteine, trityl-D-penicillamine, cystathionine, homocystine, L-homocystine, (2-aminoethyl)-L-cysteine, seleno-L-cystine, cystathionine, Cys(StBu)-OH, and acetamidomethyl-D-penicillamine.

Amino acid analogues include analogues of phenylalanine and tyrosine. Examples of amino acid analogues of phenylalanine and tyrosine include β-methyl-phenylalanine, β-hydroxyphenylalanine, α-methyl-3-methoxy-DL-phenylalanine, α-methyl-D-phenylalanine, α-methyl-L-phenylalanine, 1,2,3,4-tetrahydroisoquinoline-3-carboxylic acid, 2,4-dichloro-phenylalanine, 2-(trifluoromethyl)-D-phenylalanine, 2-(trifluoromethyl)-L-phenylalanine, 2-bromo-D-phenylalanine, 2-bromo-L-phenylalanine, 2-chloro-D-phenylalanine, 2-chloro-L-phenylalanine, 2-cyano-D-phenylalanine, 2-cyano-L-phenylalanine, 2-fluoro-D-phenylalanine, 2-fluoro-L-phenylalanine, 2-methyl-D-phenylalanine, 2-methyl-L-phenylalanine, 2-nitro-D-phenylalanine, 2-nitro-L-phenylalanine, 2;4;5-trihydroxy-phenylalanine, 3,4,5-trifluoro-D-phenylalanine, 3,4,5-trifluoro-L-phenylalanine, 3,4-dichloro-D-phenylalanine, 3,4-dichloro-L-phenylalanine, 3,4-difluoro-D-phenylalanine, 3,4-difluoro-L-phenylalanine, 3,4-dihydroxy-L-phenylalanine, 3,4-dimethoxy-L-phenylalanine, 3,5,3′-triiodo-L-thyronine, 3,5-diiodo-D-tyrosine, 3,5-diiodo-L-tyrosine, 3,5-diiodo-L-thyronine, 3-(trifluoromethyl)-D-phenylalanine, 3-(trifluoromethyl)-L-phenylalanine, 3-amino-L-tyrosine, 3-bromo-D-phenylalanine, 3-bromo-L-phenylalanine, 3-chloro-D-phenylalanine, 3-chloro-L-phenylalanine, 3-chloro-L-tyrosine, 3-cyano-D-phenylalanine, 3-cyano-L-phenylalanine, 3-fluoro-D-phenylalanine, 3-fluoro-L-phenylalanine, 3-fluoro-tyrosine, 3-iodo-D-phenylalanine, 3-iodo-L-phenylalanine, 3-iodo-L-tyrosine, 3-methoxy-L-tyrosine, 3-methyl-D-phenylalanine, 3-methyl-L-phenylalanine, 3-nitro-D-phenylalanine, 3-nitro-L-phenylalanine, 3-nitro-L-tyrosine, 4-(trifluoromethyl)-D-phenylalanine, 4-(trifluoromethyl)-L-phenylalanine, 4-amino-D-phenylalanine, 4-amino-L-phenylalanine, 4-benzoyl-D-phenylalanine, 4-benzoyl-L-phenylalanine, 4-bis(2-chloroethyl)amino-L-phenylalanine, 4-bromo-D-phenylalanine, 4-bromo-L-phenylalanine, 4-chloro-D-phenylalanine, 4-chloro-L-phenylalanine, 4-cyano-D-phenylalanine, 4-cyano-L-phenylalanine, 4-fluoro-D-phenylalanine, 4-fluoro-L-phenylalanine, 4-iodo-D-phenylalanine, 4-iodo-L-phenylalanine, homophenylalanine, thyroxine, 3,3-diphenylalanine, thyronine, ethyl-tyrosine, and methyl-tyrosine.

Amino acid analogues include analogues of proline. Examples of amino acid analogues of proline include, but are not limited to, 3,4-dehydro-proline, 4-fluoro-proline, cis-4-hydroxy-proline, thiazolidine-2-carboxylic acid, and trans-4-fluoro-proline.

Amino acid analogues include analogues of serine and threonine. Examples of amino acid analogues of serine and threonine include, but are not limited to, 3-amino-2-hydroxy-5-methylhexanoic acid, 2-amino-3-hydroxy-4-methylpentanoic acid, 2-amino-3-ethoxybutanoic acid, 2-amino-3-methoxybutanoic acid, 4-amino-3-hydroxy-6-methylheptanoic acid, 2-amino-3-benzyloxypropionic acid, 2-amino-3-benzyloxypropionic acid, 2-amino-3-ethoxypropionic acid, 4-amino-3-hydroxybutanoic acid, and α-methylserine.

Amino acid analogues include analogues of tryptophan. Examples of amino acid analogues of tryptophan include, but are not limited to, the following: α-methyl-tryptophan; β-(3-benzothienyl)-D-alanine; β-(3-benzothienyl)-L-alanine; 1-methyl-tryptophan; 4-methyl-tryptophan; 5-benzyloxy-tryptophan; 5-bromo-tryptophan; 5-chloro-tryptophan; 5-fluoro-tryptophan; 5-hydroxy-tryptophan; 5-hydroxy-L-tryptophan; 5-methoxy-tryptophan; 5-methoxy-L-tryptophan; 5-methyl-tryptophan; 6-bromo-tryptophan; 6-chloro-D-tryptophan; 6-chloro-tryptophan; 6-fluoro-tryptophan; 6-methyl-tryptophan; 7-benzyloxy-tryptophan; 7-bromo-tryptophan; 7-methyl-tryptophan; D-1,2,3,4-tetrahydro-norharman-3-carboxylic acid; 6-methoxy-1,2,3,4-tetrahydronorharman-1-carboxylic acid; 7-azatryptophan; L-1,2,3,4-tetrahydro-norharman-3-carboxylic acid; 5-methoxy-2-methyl-tryptophan; and 6-chloro-L-tryptophan.

In some embodiments, amino acid analogues are racemic. In some embodiments, the D isomer of the amino acid analogue is used. In some embodiments, the L isomer of the amino acid analogue is used. In other embodiments, the amino acid analogue comprises chiral centers that are in the R or S configuration. In still other embodiments, the amino group(s) of a β-amino acid analogue is substituted with a protecting group, e.g., tert-butyloxycarbonyl (BOC group), 9-fluorenylmethyloxycarbonyl (FMOC), tosyl, and the like. In yet other embodiments, the carboxylic acid functional group of a β-amino acid analogue is protected, e.g., as its ester derivative. In some embodiments the salt of the amino acid analogue is used.

A “non-essential” amino acid residue is a residue that can be altered from the wild-type sequence of a polypeptide without abolishing or substantially abolishing its essential biological or biochemical activity (e.g., receptor binding or activation). An “essential” amino acid residue is a residue that, when altered from the wild-type sequence of the polypeptide, results in abolishing or substantially abolishing the polypeptide's essential biological or biochemical activity.

A “conservative amino acid substitution” is one in which the amino acid residue is replaced with an amino acid residue having a similar side chain. Families of amino acid residues having similar side chains have been defined in the art. These families include amino acids with basic side chains (e.g., K, R, H), acidic side chains (e.g., D, E), uncharged polar side chains (e.g., G, N, Q, S, T, Y, C), nonpolar side chains (e.g., A, V, L, I, P, F, M, W), beta-branched side chains (e.g., T, V, I) and aromatic side chains (e.g., Y, F, W, H). Thus, a predicted nonessential amino acid residue in a polypeptide, e.g., is replaced with another amino acid residue from the same side chain family. Other examples of acceptable substitutions are substitutions based on isosteric considerations (e.g., norleucine for methionine) or other properties (e.g., 2-thienylalanine for phenylalanine, or 6-Cl-tryptophan for tryptophan).

The term “capping group” refers to the chemical moiety occurring at either the carboxy or amino terminus of the polypeptide chain of the subject peptidomimetic macrocycle. The capping group of a carboxy terminus includes an unmodified carboxylic acid (i.e. —COOH) or a carboxylic acid with a substituent. For example, the carboxy terminus can be substituted with an amino group to yield a carboxamide at the C-terminus. Various substituents include but are not limited to primary, secondary, and tertiary amines, including pegylated secondary amines. Representative secondary amine capping groups for the C-terminus include:

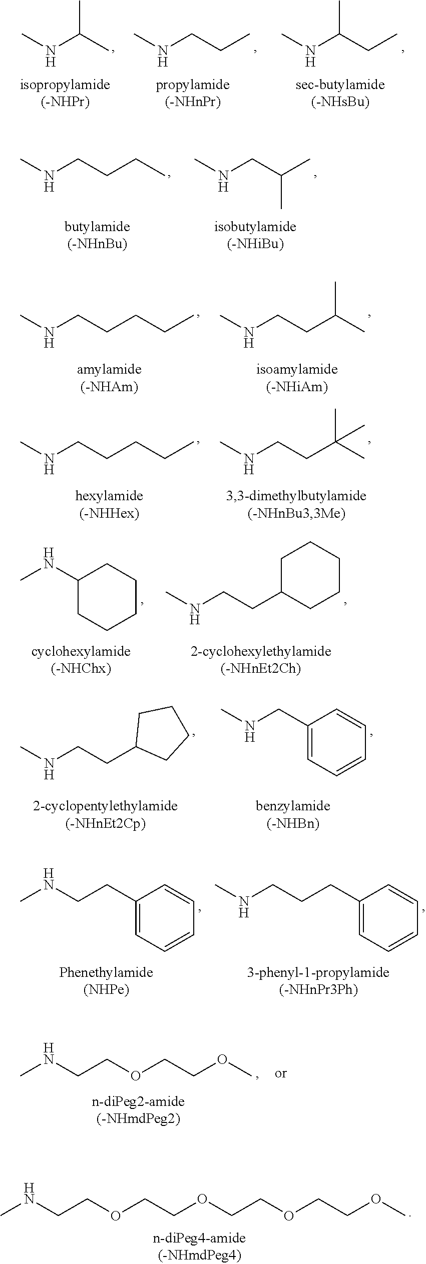

The capping group of an amino terminus includes an unmodified amine (i.e. —NH₂) or an amine with a substituent. For example, the amino terminus can be substituted with an acyl group to yield a carboxamide at the N-terminus. Various substituents include but are not limited to substituted acyl groups, including C₁-C₆ carbonyls, C₇-C₃₀ carbonyls, and pegylated carbamates. Representative capping groups for the N-terminus include, but are not limited to, 4-FBzl (4-fluoro-benzyl) and the following:

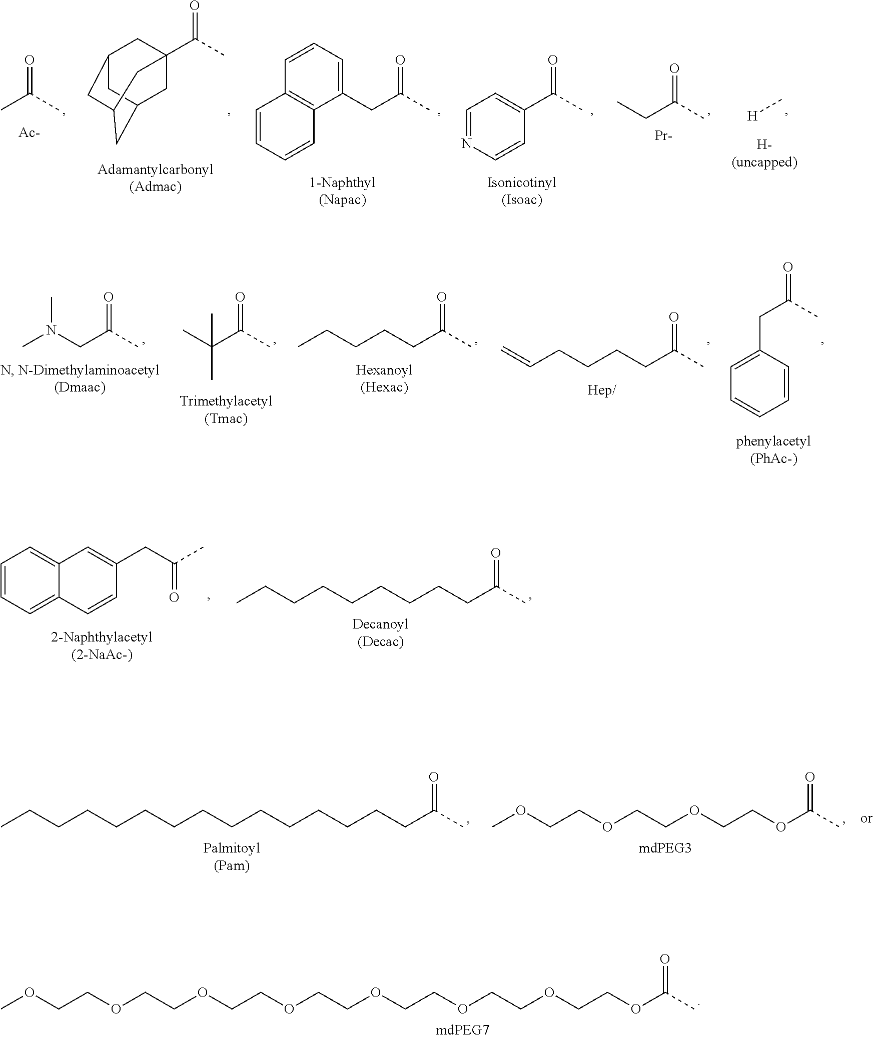

The term “member” as used herein in conjunction with macrocycles or macrocycle-forming linkers refers to the atoms that form or can form the macrocycle, and excludes substituent or side chain atoms. By analogy, cyclodecane, 1,2-difluoro-decane and 1,3-dimethyl cyclodecane are all considered ten-membered macrocycles as the hydrogen or fluoro substituents or methyl side chains do not participate in forming the macrocycle.

The symbol “ [symbol] ” when used as part of a molecular structure refers to a single bond or a trans or cis double bond.

The term “amino acid side chain” refers to a moiety attached to the α-carbon (or another backbone atom) in an amino acid. For example, the amino acid side chain for alanine is methyl, the amino acid side chain for phenylalanine is phenylmethyl, the amino acid side chain for cysteine is thiomethyl, the amino acid side chain for aspartate is carboxymethyl, the amino acid side chain for tyrosine is 4-hydroxyphenylmethyl, etc. Other non-naturally-occurring amino acid side chains are also included, for example, those that occur in nature (e.g., an amino acid metabolite) or those that are made synthetically (e.g., an α,α di-substituted amino acid).

The term “α,α di-substituted amino” acid refers to a molecule or moiety containing both an amino group and a carboxyl group bound to a carbon (the β-carbon) that is attached to two natural or non-natural amino acid side chains.

The term “polypeptide” encompasses two or more naturally- or non-naturally-occurring amino acids joined by a covalent bond (e.g., an amide bond). Polypeptides as described herein include full length proteins (e.g., fully processed proteins) as well as shorter amino acid sequences (e.g., fragments of naturally-occurring proteins or synthetic polypeptide fragments).

The term “first C-terminal amino acid” refers to the amino acid which is closest to the C-terminus. The term “second C-terminal amino acid” refers to the amino acid attached at the N-terminus of the first C-terminal amino acid.

The term “macrocyclization reagent” or “macrocycle-forming reagent” as used herein refers to any reagent which can be used to prepare a peptidomimetic macrocycle by mediating the reaction between two reactive groups. Reactive groups can be, for example, an azide and alkyne, in which case macrocyclization reagents include, without limitation, Cu reagents such as reagents which provide a reactive Cu(I) species, such as CuBr, CuI or CuOTf, as well as Cu(II) salts such as Cu(CO₂CH₃)₂, CuSO₄, and CuCl₂ that can be converted in situ to an active Cu(I) reagent by the addition of a reducing agent such as ascorbic acid or sodium ascorbate. Macrocyclization reagents can additionally include, for example, Ru reagents known in the art such as Cp*RuCl(PPh₃)₂, [Cp*RuCl]₄ or other Ru reagents which can provide a reactive Ru(II) species. In other cases, the reactive groups are terminal olefins. In such embodiments, the macrocyclization reagents or macrocycle-forming reagents are metathesis catalysts including, but not limited to, stabilized, late transition metal carbene complex catalysts such as Group VIII transition metal carbene catalysts. For example, such catalysts are Ru and Os metal centers having a +2 oxidation state, an electron count of 16 and pentacoordinated. In other examples, catalysts have W or Mo centers. In some embodiments, the reactive groups are thiol groups. In some embodiments, the macrocyclization reagent is, for example, a linker functionalized with two thiol-reactive groups such as halogen groups.

The term “halo” or “halogen” refers to fluorine, chlorine, bromine or iodine or a radical thereof.

The term “alkyl” refers to a hydrocarbon chain that is a straight chain or branched chain, containing the indicated number of carbon atoms. For example, C₁-C₁₀ indicates that the group has from 1 to 10 (inclusive) carbon atoms in it. In the absence of any numerical designation, “alkyl” is a chain (straight or branched) having 1 to 20 (inclusive) carbon atoms.

The term “alkylene” refers to a divalent alkyl (i.e., —R—).

The term “alkenyl” refers to a hydrocarbon chain that is a straight chain or branched chain having one or more carbon-carbon double bonds. The alkenyl moiety contains the indicated number of carbon atoms. For example, C₂-C₁₀ indicates that the group has from 2 to 10 (inclusive) carbon atoms. The term “lower alkenyl” refers to a C₂-C₆ alkenyl chain. In the absence of any numerical designation, “alkenyl” is a chain (straight or branched) having 2 to 20 (inclusive) carbon atoms.

The term “alkynyl” refers to a hydrocarbon chain that is a straight chain or branched chain having one or more carbon-carbon triple bonds. The alkynyl moiety contains the indicated number of carbon atoms. For example, C₂-C₁₀ indicates that the group has from 2 to 10 (inclusive) carbon atoms. The term “lower alkynyl” refers to a C₂-C₆ alkynyl chain. In the absence of any numerical designation, “alkynyl” is a chain (straight or branched) having 2 to 20 (inclusive) carbon atoms.

The term “aryl” refers to a 6-carbon monocyclic or 10-carbon bicyclic aromatic ring system wherein 0, 1, 2, 3, or 4 atoms of each ring are substituted by a substituent. Examples of aryl groups include phenyl, naphthyl and the like. The term “arylalkoxy” refers to an alkoxy substituted with aryl.

“Arylalkyl” refers to an aryl group, as defined above, wherein one of the aryl group's hydrogen atoms has been replaced with a C₁-C₅ alkyl group, as defined above. Representative examples of an arylalkyl group include, but are not limited to, 2-methylphenyl, 3-methylphenyl, 4-methylphenyl, 2-ethylphenyl, 3-ethylphenyl, 4-ethylphenyl, 2-propylphenyl, 3-propylphenyl, 4-propylphenyl, 2-butylphenyl, 3-butylphenyl, 4-butylphenyl, 2-pentylphenyl, 3-pentylphenyl, 4-pentylphenyl, 2-isopropylphenyl, 3-isopropylphenyl, 4-isopropylphenyl, 2-isobutylphenyl, 3-isobutylphenyl, 4-isobutylphenyl, 2-sec-butylphenyl, 3-sec-butylphenyl, 4-sec-butylphenyl, 2-t-butylphenyl, 3-t-butylphenyl and 4-t-butylphenyl.

“Arylamido” refers to an aryl group, as defined above, wherein one of the aryl group's hydrogen atoms has been replaced with one or more —C(O)NH₂ groups. Representative examples of an arylamido group include 2-C(O)NH₂-phenyl, 3-C(O)NH₂-phenyl, 4-C(O)NH₂-phenyl, 2-C(O)NH₂-pyridyl, 3-C(O)NH₂-pyridyl, and 4-C(O)NH₂-pyridyl.

“Alkylheterocycle” refers to a C₁-C₅ alkyl group, as defined above, wherein one of the C₁-C₅ alkyl group's hydrogen atoms has been replaced with a heterocycle. Representative examples of an alkylheterocycle group include, but are not limited to, —CH₂CH₂-morpholine, —CH₂CH₂-piperidine, —CH₂CH₂CH₂-morpholine, and —CH₂CH₂CH₂-imidazole.

“Alkylamido” refers to a C₁-C₅ alkyl group, as defined above, wherein one of the C₁-C₅ alkyl group's hydrogen atoms has been replaced with a —C(O)NH₂ group. Representative examples of an alkylamido group include, but are not limited to, —CH₂—C(O)NH₂, —CH₂CH₂—C(O)NH₂, —CH₂CH₂CH₂C(O)NH₂, —CH₂CH₂CH₂CH₂C(O)NH₂, —CH₂CH₂CH₂CH₂CH₂C(O)NH₂, —CH₂CH(C(O)NH₂)CH₃, —CH₂CH(C(O)NH₂)CH₂CH₃, —CH(C(O)NH₂)CH₂CH₃, —C(CH₃)₂CH₂C(O)NH₂, —CH₂—CH₂—NH—C(O)—CH₃, —CH₂—CH₂—NH—C(O)—CH₃—CH3, and —CH₂—CH₂—NH—C(O)—CH═CH₂.

“Alkanol” refers to a C₁-C₅ alkyl group, as defined above, wherein one of the C₁-C₅ alkyl group's hydrogen atoms has been replaced with a hydroxyl group. Representative examples of an alkanol group include, but are not limited to, —CH₂OH, —CH₂CH₂OH, —CH₂CH₂CH₂OH, —CH₂CH₂CH₂CH₂OH, —CH₂CH₂CH₂ CH₂CH₂OH, —CH₂CH(OH)CH₃, —CH₂CH(OH)CH₂CH₃, —CH(OH)CH₃ and —C(CH₃)₂CH₂OH.

“Alkylcarboxy” refers to a C₁-C₅ alkyl group, as defined above, wherein one of the C₁-C₅ alkyl group's hydrogen atoms has been replaced with a —COOH group. Representative examples of an alkylcarboxy group include, but are not limited to, —CH₂COOH, —CH₂CH₂COOH, —CH₂CH₂CH₂COOH, —CH₂CH₂CH₂CH₂COOH, —CH₂CH(COOH)CH₃, —CH₂CH₂CH₂CH₂CH₂COOH, —CH₂CH(COOH)CH₂CH₃, —CH(COOH)CH₂CH₃ and —C(CH₃)₂CH₂COOH.

The term “cycloalkyl” as employed herein includes saturated and partially unsaturated cyclic hydrocarbon groups having 3 to 12 carbons, preferably 3 to 8 carbons, and more preferably 3 to 6 carbons, wherein the cycloalkyl group additionally is optionally substituted. Some cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of O, N, or S if monocyclic, bicyclic, or tricyclic, respectively), wherein 0, 1, 2, 3, or 4 atoms of each ring are substituted by a substituent. Examples of heteroaryl groups include pyridyl, furyl or furanyl, imidazolyl, benzimidazolyl, pyrimidinyl, thiophenyl or thienyl, quinolinyl, indolyl, thiazolyl, and the like.

The term “heteroarylalkyl” or the term “heteroaralkyl” refers to an alkyl substituted with a heteroaryl. The term “heteroarylalkoxy” refers to an alkoxy substituted with heteroaryl.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of O, N, or S if monocyclic, bicyclic, or tricyclic, respectively), wherein 0, 1, 2 or 3 atoms of each ring are substituted by a substituent. Examples of heterocyclyl groups include piperazinyl, pyrrolidinyl, dioxanyl, morpholinyl, tetrahydrofuranyl, and the like.

The term “substituent” refers to a group replacing a second atom or group such as a hydrogen atom on any molecule, compound or moiety. Suitable substituents include, without limitation, halo, hydroxy, mercapto, oxo, nitro, haloalkyl, alkyl, alkaryl, aryl, aralkyl, alkoxy, thioalkoxy, aryloxy, amino, alkoxycarbonyl, amido, carboxy, alkanesulfonyl, alkylcarbonyl, and cyano groups.

In some embodiments, the compounds disclosed herein contain one or more asymmetric centers and thus occur as racemates and racemic mixtures, single enantiomers, individual diastereomers and diastereomeric mixtures. All such isomeric forms of these compounds are included unless expressly provided otherwise. In some embodiments, the compounds disclosed herein are also represented in multiple tautomeric forms, in such instances, the compounds include all tautomeric forms of the compounds described herein (e.g., if alkylation of a ring system results in alkylation at multiple sites, the invention includes all such reaction products). All such isomeric forms of such compounds are included unless expressly provided otherwise. All crystal forms of the compounds described herein are included unless expressly provided otherwise.

As used herein, the terms “increase” and “decrease” mean, respectively, to cause a statistically significantly (i.e., p<0.1) increase or decrease of at least 5%.

As used herein, the recitation of a numerical range for a variable is intended to convey that the variable is equal to any of the values within that range. Thus, for a variable which is inherently discrete, the variable is equal to any integer value within the numerical range, including the end-points of the range. Similarly, for a variable which is inherently continuous, the variable is equal to any real value within the numerical range, including the end-points of the range. As an example, and without limitation, a variable which is described as having values between 0 and 2 takes the values 0, 1 or 2 if the variable is inherently discrete, and takes the values 0.0, 0.1, 0.01, 0.001, or any other real values ≥0 and ≤2 if the variable is inherently continuous.

As used herein, unless specifically indicated otherwise, the word “or” is used in the inclusive sense of “and/or” and not the exclusive sense of “either/or”.

The term “on average” represents the mean value derived from performing at least three independent replicates for each data point.

The term “biological activity” encompasses structural and functional properties of a macrocycle. Biological activity is, for example, structural stability, alpha-helicity, affinity for a target, resistance to proteolytic degradation, cell penetrability, intracellular stability, in vivo stability, or any combination thereof.

The term “binding affinity” refers to the strength of a binding interaction, for example between a peptidomimetic macrocycle and a target. Binding affinity can be expressed, for example, as equilibrium dissociation constant (“K_{D}”), which is expressed in units which are a measure of concentration (e.g. M, mM, μM, nM etc). Numerically, binding affinity and K_{D} values vary inversely, such that a lower binding affinity corresponds to a higher K_{D} value, while a higher binding affinity corresponds to a lower K_{D} value. Where high binding affinity is desirable, “improved” binding affinity refers to higher binding affinity and therefore lower K_{D} values.

As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease.

The terms “combination therapy” or “combined treatment” or in “combination” as used herein denotes any form of concurrent or parallel treatment with at least two distinct therapeutic agents.

The term “in vitro efficacy” refers to the extent to which a test compound, such as a peptidomimetic macrocycle, produces a beneficial result in an in vitro test system or assay. In vitro efficacy can be measured, for example, as an “IC₅₀” or “EC₅₀” value, which represents the concentration of the test compound which produces 50% of the maximal effect in the test system.

The term “ratio of in vitro efficacies” or “in vitro efficacy ratio” refers to the ratio of IC₅₀ or EC₅₀ values from a first assay (the numerator) versus a second assay (the denominator). Consequently, an improved in vitro efficacy ratio for Assay 1 versus Assay 2 refers to a lower value for the ratio expressed as IC₅₀(Assay 1)/IC₅₀(Assay 2) or alternatively as EC₅₀(Assay 1)/EC₅₀(Assay 2). This concept can also be characterized as “improved selectivity” in Assay 1 versus Assay 2, which can be due either to a decrease in the IC₅₀ or EC₅₀ value for Target 1 or an increase in the value for the IC₅₀ or EC₅₀ value for Target 2.

As used in the present application, “biological sample” means any fluid or other material derived from the body of a normal or diseased subject, such as blood, serum, plasma, lymph, urine, saliva, tears, cerebrospinal fluid, milk, amniotic fluid, bile, ascites fluid, pus, and the like. Also included within the meaning of the term “biological sample” is an organ or tissue extract and culture fluid in which any cells or tissue preparation from a subject has been incubated. The biological samples can be any samples from which genetic material can be obtained. Biological samples can also include solid or liquid cancer cell samples or specimens. The cancer cell sample can be a cancer cell tissue sample. In some embodiments, the cancer cell tissue sample can obtained from surgically excised tissue. Exemplary sources of biological samples include fine needle aspiration, core needle biopsy, vacuum assisted biopsy, incisional biopsy, excisional biopsy, punch biopsy, shave biopsy or skin biopsy. In some cases, the biological samples comprise fine needle aspiration samples. In some embodiments, the biological samples comprise tissue samples, including, for example, excisional biopsy, incisional biopsy, or other biopsy. The biological samples can comprise a mixture of two or more sources; for example, fine needle aspirates and tissue samples. Tissue samples and cellular samples can also be obtained without invasive surgery, for example by punctuating the chest wall or the abdominal wall or from masses of breast, thyroid or other sites with a fine needle and withdrawing cellular material (fine needle aspiration biopsy). In some embodiments, a biological sample is a bone marrow aspirate sample. A biological sample can be obtained by methods known in the art such as the biopsy methods provided herein, swabbing, scraping, phlebotomy, or any other suitable method.

The term “solid tumor” or “solid cancer” as used herein refers to tumors that usually do not contain cysts or liquid areas. Solid tumors as used herein include sarcomas, carcinomas and lymphomas. In various embodiments, leukemia (cancer of blood) is not solid tumor.

Solid tumor cancers that can be treated by the methods provided herein include, but are not limited to, sarcomas, carcinomas, and lymphomas. In specific embodiments, solid tumors that can be treated in accordance with the methods described include, but are not limited to, cancer of the breast, liver, neuroblastoma, head, neck, eye, mouth, throat, esophagus, esophagus, chest, bone, lung, kidney, colon, rectum or other gastrointestinal tract organs, stomach, spleen, skeletal muscle, subcutaneous tissue, prostate, breast, ovaries, testicles or other reproductive organs, skin, thyroid, blood, lymph nodes, kidney, liver, pancreas, and brain or central nervous system. Solid tumors that can be treated by the instant methods include tumors and/or metastasis (wherever located) other than lymphatic cancer, for example brain and other central nervous system tumors (including but not limited to tumors of the meninges, brain, spinal cord, cranial nerves and other parts of central nervous system, e.g. glioblastomas or medulla blastemas); head and/or neck cancer; breast tumors; circulatory system tumors (including but not limited to heart, mediastinum and pleura, and other intrathoracic organs, vascular tumors and tumor-associated vascular tissue); excretory system tumors (including but not limited to tumors of kidney, renal pelvis, ureter, bladder, other and unspecified urinary organs); gastrointestinal tract tumors (including but not limited to tumors of the esophagus, stomach, small intestine, colon, colorectal, rectosigmoid junction, rectum, anus and anal canal, tumors involving the liver and intrahepatic bile ducts, gall bladder, other and unspecified parts of biliary tract, pancreas, other and digestive organs); oral cavity tumors (including but not limited to tumors of lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx); reproductive system tumors (including but not limited to tumors of vulva, vagina, Cervix uteri, Corpus uteri, uterus, ovary, and other sites associated with female genital organs, placenta, penis, prostate, testis, and other sites associated with male genital organs); respiratory tract tumors (including but not limited to tumors of nasal cavity and middle ear, accessory sinuses, larynx, trachea, bronchus and lung, e.g. small cell lung cancer or non-small cell lung cancer); skeletal system tumors (including but not limited to tumors of bone and articular cartilage of limbs, bone articular cartilage and other sites); skin tumors (including but not limited to malignant melanoma of the skin, non-melanoma skin cancer, basal cell carcinoma of skin, squamous cell carcinoma of skin, mesothelioma, Kaposi's sarcoma); and tumors involving other tissues including peripheral nerves and autonomic nervous system, connective and soft tissue, retroperitoneum and peritoneum, eye and adnexa, thyroid, adrenal gland and other endocrine glands and related structures, secondary and unspecified malignant neoplasm of lymph nodes, secondary malignant neoplasm of respiratory and digestive systems and secondary malignant neoplasm of other sites.

In some examples, the solid tumor treated by the methods of the instant disclosure is pancreatic cancer, bladder cancer, colon cancer, liver cancer, colorectal cancer (colon cancer or rectal cancer), breast cancer, prostate cancer, renal cancer, hepatocellular cancer, lung cancer, ovarian cancer, cervical cancer, gastric cancer, esophageal cancer, head and neck cancer, melanoma, neuroendocrine cancers, CNS cancers, brain tumors, bone cancer, skin cancer, ocular tumor, choriocarcinoma (tumor of the placenta), sarcoma or soft tissue cancer.

In some examples, the solid tumor to be treated by the methods of the instant disclosure is selected bladder cancer, bone cancer, breast cancer, cervical cancer, CNS cancer, colon cancer, ocular tumor, renal cancer, liver cancer, lung cancer, pancreatic cancer, choriocarcinoma (tumor of the placenta), prostate cancer, sarcoma, skin cancer, soft tissue cancer or gastric cancer.

In some examples, the solid tumor treated by the methods of the instant disclosure is breast cancer. Non limiting examples of breast cancer that can be treated by the instant methods include ductal carcinoma in situ (DCIS or intraductal carcinoma), lobular carcinoma in situ (LCIS), invasive (or infiltrating) ductal carcinoma, invasive (or infiltrating) lobular carcinoma, inflammatory breast cancer, triple-negative breast cancer, paget disease of the nipple, phyllodes tumor (phylloides tumor or cystosarcoma phyllodes), angiosarcoma, adenoid cystic (or adenocystic) carcinoma, low-grade adenosquamous carcinoma, medullary carcinoma, papillary carcinoma, tubular carcinoma, metaplastic carcinoma, micropapillary carcinoma, and mixed carcinoma.

In some examples, the solid tumor treated by the methods of the instant disclosure is bone cancer. Non limiting examples of bone cancer that can be treated by the instant methods include osteosarcoma, chondrosarcoma, the Ewing Sarcoma Family of Tumors (ESFTs).

In some examples, the solid tumor treated by the methods of the instant disclosure is skin cancer. Non limiting examples of skin cancer that can be treated by the instant methods include melanoma, basal cell skin cancer, and squamous cell skin cancer.

In some examples, the solid tumor treated by the methods of the instant disclosure is ocular tumor. Non limiting examples of ocular tumor that can be treated by the methods of the instant disclosure include ocular tumor is choroidal nevus, choroidal melanoma, choroidal metastasis, choroidal hemangioma, choroidal osteoma, iris melanoma, uveal melanoma, intraocular lymphoma, melanocytoma, metastasis retinal capillary hemangiomas, congenital hypertrophy of the RPE, RPE adenoma or retinoblastoma.

In some embodiments solid tumors treated by the methods disclosed herein exclude cancers that are known to be associated with HPV (Human papillomavirus). The excluded group includes HPV positive cervical cancer, HPV positive anal cancer, and HPV head and neck cancers, such as oropharyngeal cancers.

The term “liquid cancer” as used herein refers to cancer cells that are present in body fluids, such as blood, lymph and bone marrow. Liquid cancers include leukemia, myeloma and liquid lymphomas. Liquid lymphomas include lymphomas that contain cysts or liquid areas. Liquid cancers as used herein do not include solid tumors, such as sarcomas and carcinomas or solid lymphomas that do not contain cysts or liquid areas.

Liquid cancer cancers that can be treated by the methods provided herein include, but are not limited to, leukemias, myelomas, and liquid lymphomas. In specific embodiments, liquid cancers that can be treated in accordance with the methods described include, but are not limited to, liquid lymphomas, lekemias, and myelomas. Exemplary liquid lymphomas and leukemias that can be treated in accordance with the methods described include, but are not limited to, chronic lymphocytic leukemia/small lymphocytic lymphoma, B-cell prolymphocytic leukemia, lymphoplasmacytic lymphoma (such as waldenstrom macroglobulinemia), splenic marginal zone lymphoma, plasma cell myeloma, plasmacytoma, monoclonal immunoglobulin deposition diseases, heavy chain diseases, extranodal marginal zone B cell lymphoma, also called malt lymphoma, nodal marginal zone B cell lymphoma (nmzl), follicular lymphoma, mantle cell lymphoma, diffuse large B cell lymphoma, mediastinal (thymic) large B cell lymphoma, intravascular large B cell lymphoma, primary effusion lymphoma, burkitt lymphoma/leukemia, T cell prolymphocytic leukemia, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides/sezary syndrome, primary cutaneous CD30-positive T cell lymphoproliferative disorders, primary cutaneous anaplastic large cell lymphoma, lymphomatoid papulosis, angioimmunoblastic T cell lymphoma, peripheral T cell lymphoma, unspecified, anaplastic large cell lymphoma, classical Hodgkin lymphomas (nodular sclerosis, mixed cellularity, lymphocyte-rich, lymphocyte depleted or not depleted), and nodular lymphocyte-predominant Hodgkin lymphoma.

Examples of liquid cancers include cancers involving hyperplastic/neoplastic cells of hematopoietic origin, e.g., arising from myeloid, lymphoid or erythroid lineages, or precursor cells thereof. Exemplary disorders include: acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), multiple mylenoma, hairy cell leukemia (HLL) and Waldenstrom's macroglobulinemia (WM). Additional forms of malignant liquid lymphomas include, but are not limited to non-Hodgkin lymphoma and variants thereof, adult T cell leukemia/lymphoma (ATL), cutaneous T-cell lymphoma (CTCL), periphieral T-cell lymphoma (PTCL), large granular lymphocytic leukemia (LGF), Hodgkin's disease and Reed-Sternberg disease. For example, liquid cancers include, but are not limited to, acute lymphocytic leukemia (ALL); T-cell acute lymphocytic leukemia (T-ALL); anaplastic large cell lymphoma (ALCL); chronic myelogenous leukemia (CML); acute myeloid leukemia (AML); chronic lymphocytic leukemia (CLL); B-cell chronic lymphocytic leukemia (B-CLL); diffuse large B-cell lymphomas (DLBCL); hyper eosinophilia/chronic eosinophilia; and Burkitt's lymphoma.

In some embodiments, the cancer comprises an acute lymphoblastic leukemia; acute myeloid leukemia; AIDS-related cancers; AIDS-related lymphoma; chronic lymphocytic leukemia; chronic myelogenous leukemia; chronic myeloproliferative disorders; adult T cell leukemia/lymphoma (ATL), cutaneous T-cell lymphoma (CTCL), peripheral T-cell lymphoma (PTCL); Hodgkin lymphoma; multiple myeloma; multiple myeloma/plasma cell neoplasm; Non-Hodgkin lymphoma; or primary central nervous system (CNS) lymphoma. In various embodiments, the liquid cancer can be B-cell chronic lymphocytic leukemia, B-cell lymphoma-DLBCL, B-cell lymphoma-DLBCL-germinal center-like, B-cell lymphoma-DLBCL-activated B-cell-like, or Burkitt's lymphoma.

In some embodiments, a subject treated in accordance with the methods provided herein is a human who has or is diagnosed with cancer lacking p53 deactivating mutation and/or expressing wild type p53. In some embodiments, a subject treated for cancer in accordance with the methods provided herein is a human predisposed or susceptible to cancer lacking p53 deactivating mutation and/or expressing wild type p53. In some embodiments, a subject treated for cancer in accordance with the methods provided herein is a human at risk of developing cancer lacking p53 deactivating mutation and/or expressing wild type p53. A p53 deactivating mutation in some example can be a mutation in DNA-binding domain of the p53 protein. In some examples the p53 deactivating mutation can be a missense mutation. In various examples, the cancer can be determined to lack one or more p53 deactivating mutations selected from mutations at one or more of residues R175, G245, R248, R249, R273, and R282. The lack of p53 deactivating mutation and/or the presence of wild type p53 in the cancer can be determined by any suitable method known in art, for example by sequencing, array based testing, RNA analysis and amplifications methods like PCR.

In certain embodiments, the human subject is refractory and/or intolerant to one or more other standard treatment of the cancer known in art. In some embodiments, the human subject has had at least one unsuccessful prior treatment and/or therapy of the cancer.

In some embodiments, a subject treated for tumor in accordance with the methods provided herein is a human, who has or is diagnosed with a tumor. In other embodiments, a subject treated for tumor in accordance with the methods provided herein is a human, predisposed or susceptible to a tumor. In some embodiments, a subject treated for tumor in accordance with the methods provided herein is a human, at risk of developing a tumor.

In some embodiments, a subject treated for tumor in accordance with the methods provided herein is a human, who has or is diagnosed with a tumor, determined to lack a p53 deactivating mutation and/or expressing wild type p53. In other embodiments, a subject treated for tumor in accordance with the methods provided herein is a human, predisposed or susceptible to a tumor, determined to lack a p53 deactivating mutation and/or expressing wild type p53. In some embodiments, a subject treated for tumor in accordance with the methods provided herein is a human, at risk of developing a tumor, determined to lack a p53 deactivating mutation and/or expressing wild type p53. A p53 deactivating mutation, as used herein is any mutation that leads to loss of (or a decrease in) the in vitro apoptotic activity of p53.

In some embodiments, the subject treated for tumor in accordance with the methods provided herein is a human, who has or is diagnosed with a tumor, determined to have a p53 gain of function mutation. In other embodiments, a subject treated for tumor in accordance with the methods provided herein is a human, predisposed or susceptible to a tumor, determined to have a p53 gain of function mutation. In some embodiments, a subject treated for tumor in accordance with the methods provided herein is a human, at risk of developing a tumor, determined to have a p53 gain of function mutation. A p53 gain of function mutation, as used herein is any mutation such that the mutant p53 exerts oncogenic functions beyond their negative domination over the wild-type p53 tumor suppressor functions. The p53 gain of function mutant protein mat exhibit new activities that can contribute actively to various stages of tumor progression and to increased resistance to anticancer treatments. Accordingly, in some embodiments, a subject with a tumor in accordance with the composition as provided herein is a human who has or is diagnosed with a tumor that is determined to have a p53 gain of function mutation.

In some embodiments, the subject treated for tumor in accordance with the methods provided herein is a human, who has or is diagnosed with a tumor that is not p53 negative. In other embodiments, a subject treated for tumor in accordance with the methods provided herein is a human, predisposed or susceptible to a tumor that is not p53 negative. In some embodiments, a subject treated for tumor in accordance with the methods provided herein is a human, at risk of developing a tumor that is not p53 negative.

In some embodiments, the subject treated for tumor in accordance with the methods provided herein is a human, who has or is diagnosed with a tumor that expresses p53 with partial loss of function mutation. In other embodiments, a subject treated for tumor in accordance with the methods provided herein is a human, predisposed or susceptible to a tumor that expresses p53 with partial loss of function mutation. In some embodiments, a subject treated for tumor in accordance with the methods provided herein is a human, at risk of developing a tumor that expresses p53 with partial loss of function mutation. As used herein “a partial loss of p53 function” mutation means that the mutant p53 exhibits some level of function of normal p53, but to a lesser or slower extent. For example, a partial loss of p53 function can mean that the cells become arrested in cell division to a lesser or slower extent.

In some embodiments, the subject treated for tumor in accordance with the methods provided herein is a human, who has or is diagnosed with a tumor that expresses p53 with a copy loss mutation and a deactivating mutation. In other embodiments, a subject treated for tumor in accordance with the methods provided herein is a human, predisposed or susceptible to a tumor that expresses p53 with a copy loss mutation and a deactivating mutation. In some embodiments, a subject treated for tumor in accordance with the methods provided herein is a human, at risk of developing a tumor that expresses p53 with a copy loss mutation and a deactivating mutation.

In some embodiments, the subject treated for tumor in accordance with the methods provided herein is a human, who has or is diagnosed with a tumor that expresses p53 with a copy loss mutation. In other embodiments, a subject treated for tumor in accordance with the methods provided herein is a human, predisposed or susceptible to a tumor that expresses p53 with a copy loss mutation. In some embodiments, a subject treated for tumor in accordance with the methods provided herein is a human, at risk of developing a tumor that expresses p53 with a copy loss mutation.

In some embodiments, the subject treated for tumor in accordance with the methods provided herein is a human, who has or is diagnosed with a tumor that expresses p53 with one or more silent mutations. In other embodiments, a subject treated for tumor in accordance with the methods provided herein is a human, predisposed or susceptible to a tumor that expresses p53 with one or more silent mutations. In some embodiments, a subject treated for tumor in accordance with the methods provided herein is a human, at risk of developing a tumor that expresses p53 with one or more silent mutations. Silent mutations as used herein are mutations which cause no change in the encoded p53 amino acid sequence.

In some embodiments, a subject treated for tumor in accordance with the methods provided herein is a human, who has or is diagnosed with a tumor, determined to lack a dominant p53 deactivating mutation. Dominant p53 deactivating mutation or dominant negative mutation, as used herein, is a mutation wherein the mutated p53 inhibits or disrupt the activity of the wild-type p53 gene.

### Peptidomimetic Macrocycles

In some embodiments, a peptidomimetic macrocycle has the Formula (I):

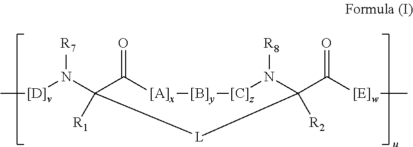

wherein:
each A, C, D, and E is independently a natural or non-natural amino acid or an amino acid analog, and each terminal D and E independently optionally includes a capping group;
each B is independently a natural or non-natural amino acid, an amino acid analog,

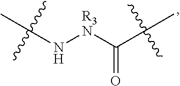

[—NH-L₃-CO—], [—NH-L₃-SO₂—], or [—NH-L₃-];
each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-, or at least one of R₁ and R₂ forms a macrocycle-forming linker L′ connected to the alpha position of one of said D or E amino acids;
each R₃ is independently hydrogen, alkyl, alkenyl, alkynyl, arylalkyl, heteroalkyl, cycloalkyl, heterocycloalkyl, cycloalkylalkyl, aryl, or heteroaryl, optionally substituted with R₅;
each L and L′ is independently a macrocycle-forming linker of the formula -L₁-L₂-;
each L₁, L₂, and L₃ is independently alkylene, alkenylene, alkynylene, heteroalkylene, cycloalkylene, heterocycloalkylene, arylene, heteroarylene, or [—R₄—K—R₄-]ₙ, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
each R₇ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
each R₈ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
each v and w is independently an integer from 1-1000, for example 1-500, 1-200, 1-100, 1-50, 1-30, 1-20, or 1-10;
u is an integer from 1-10, for example 1-5, 1-3 or 1-2;
each x, y, and z is independently an integer from 0-10, for example the sum of x+y+z is 2, 3, or 6; and
n is an integer from 1-5.

In some embodiments, v and w are integers from 1-30. In some embodiments, w is an integer from 3-1000, for example 3-500, 3-200, 3-100, 3-50, 3-30, 3-20, or 3-10. In some embodiments, the sum of x+y+z is 3 or 6. In some embodiments, the sum of x+y+z is 3. In other embodiments, the sum of x+y+z is 6.

In some embodiments, w is an integer from 3-10, for example 3-6, 3-8, 6-8, or 6-10. In some embodiments, w is 3. In other embodiments, w is 6. In some embodiments, v is an integer from 1-1000, for example 1-500, 1-200, 1-100, 1-50, 1-30, 1-20, or 1-10. In some embodiments, v is 2.

In an embodiment of any of the Formulas described herein, L₁ and L₂, either alone or in combination, do not form a triazole or a thioether.

In one example, at least one of R₁ and R₂ is alkyl that is unsubstituted or substituted with halo-. In another example, both R₁ and R₂ are independently alkyl that is unsubstituted or substituted with halo-. In some embodiments, at least one of R₁ and R₂ is methyl. In other embodiments, R₁ and R₂ are methyl.

In some embodiments, x+y+z is at least 3. In other embodiments, x+y+z is 1, 2, 3, 4, 5, 6, 7, 8, 9 or 10. In some embodiments, the sum of x+y+z is 3 or 6. In some embodiments, the sum of x+y+z is 3. In other embodiments, the sum of x+y+z is 6. Each occurrence of A, B, C, D or E in a macrocycle or macrocycle precursor is independently selected. For example, a sequence represented by the formula [A]ₓ, when x is 3, encompasses embodiments wherein the amino acids are not identical, e.g. Gln-Asp-Ala as well as embodiments wherein the amino acids are identical, e.g. Gln-Gln-Gln. This applies for any value of x, y, or z in the indicated ranges. Similarly, when u is greater than 1, each compound can encompass peptidomimetic macrocycles which are the same or different. For example, a compound can comprise peptidomimetic macrocycles comprising different linker lengths or chemical compositions.

In some embodiments, the peptidomimetic macrocycle comprises a secondary structure which is an α-helix and R₈ is —H, allowing for intra-helical hydrogen bonding. In some embodiments, at least one of A, B, C, D or E is an α,α-disubstituted amino acid. In one example, B is an α,α-disubstituted amino acid. For instance, at least one of A, B, C, D or E is 2-aminoisobutyric acid. In other embodiments, at least one of A, B, C, D or E is

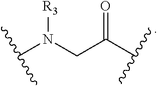

In other embodiments, the length of the macrocycle-forming linker L as measured from a first Cα to a second Cα is selected to stabilize a desired secondary peptide structure, such as an α-helix formed by residues of the peptidomimetic macrocycle including, but not necessarily limited to, those between the first Cα to a second Cα.

In some embodiments, peptidomimetic macrocycles are also provided of the formula:

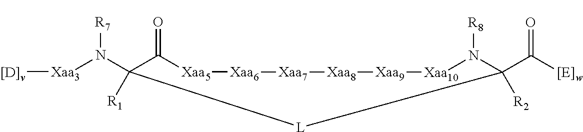

wherein:
each of Xaa₃, Xaa₅, Xaa₆, Xaa₇, Xaa₈, Xaa₉, and Xaa₁₀ is individually an amino acid, wherein at least three of Xaa₃, Xaa₅, Xaa₆, Xaa₇, Xaa₈, Xaa₉, and Xaa₁₀ are the same amino acid as the amino acid at the corresponding position of the sequence Phe₃-X₄-His₅-Tyr₆-Trp₇-Ala₈-Gln₉-Leu₁₀-X₁₁-Ser₁₂ (SEQ ID NO: 8), wherein each X is an amino acid;
each D and E is independently a natural or non-natural amino acid or an amino acid analog;
R₁ and R₂ are independently —H, alkyl, alkenyl, alkynyl, arylalkyl, cycloalkyl, cycloalkylalkyl, heteroalkyl, or heterocycloalkyl, unsubstituted or substituted with halo-; or at least one of R₁ and R₂ forms a macrocycle-forming linker L′ connected to the alpha position of one of said D or E amino acids;
each L and L′ is independently a macrocycle-forming linker of the formula -L₁-L₂-;
each L₁ and L₂ is independently alkylene, alkenylene, alkynylene, heteroalkylene, cycloalkylene, heterocycloalkylene, arylene, heteroarylene, or [—R₄—K—R₄-]ₙ, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
R₇ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
R₈ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
v is an integer from 1-1000, for example 1-500, 1-200, 1-100, 1-50, 1-30, 1-20 or 1-10;
w is an integer from 3-1000, for example 3-500, 3-200, 3-100, 3-50, 3-30, 3-20, or 3-10; and
n is an integer from 1-5.

In some embodiments, v and w are integers from 1-30. In some embodiments, w is an integer from 3-1000, for example 3-500, 3-200, 3-100, 3-50, 3-30, 3-20, or 3-10. In some embodiments, the sum of x+y+z is 3 or 6. In some embodiments, the sum of x+y+z is 3. In other embodiments, the sum of x+y+z is 6.

In some embodiments of any of the Formulas described herein, at least three of Xaa₃, Xaa₅, Xaa₆, Xaa₇, Xaa₈, Xaa₉, and Xaa₁₀ are the same amino acid as the amino acid at the corresponding position of the sequence Phe₃-X₄-His₅-Tyr₆-Trp₇-Ala₈-Gln₉-Leu₁₀-X₁₁-Ser₁₂ (SEQ ID NO: 8). In other embodiments, at least four of Xaa₃, Xaa₅, Xaa₆, Xaa₇, Xaa₈, Xaa₉, and Xaa₁₀ are the same amino acid as the amino acid at the corresponding position of the sequence Phe₃-X₄-His₅-Tyr₆-Trp₇-Ala₈-Gln₉-Leu₁₀-X₁₁-Ser₁₂ (SEQ ID NO: 8). In other embodiments, at least five of Xaa₃, Xaa₅, Xaa₆, Xaa₇, Xaa₈, Xaa₉, and Xaa₁₀ are the same amino acid as the amino acid at the corresponding position of the sequence Phe₃-X₄-His₅-Tyr₆-Trp₇-Ala₈-Gln₉-Leu₁₀-X₁₁-Ser₁₂ (SEQ ID NO: 8). In other embodiments, at least six of Xaa₃, Xaa₅, Xaa₆, Xaa₇, Xaa₈, Xaa₉, and Xaa₁₀ are the same amino acid as the amino acid at the corresponding position of the sequence Phe₃-X₄-His₅-Tyr₆-Trp₇-Ala₈-Gln₉-Leu₁₀-X₁₁-Ser₁₂ (SEQ ID NO: 8). In other embodiments, at least seven of Xaa₃, Xaa₅, Xaa₆, Xaa₇, Xaa₈, Xaa₉, and Xaa₁₀ are the same amino acid as the amino acid at the corresponding position of the sequence Phe₃-X₄-His₅-Tyr₆-Trp₇-Ala₈-Gln₉-Leu₁₀-X₁₁-Ser₁₂ (SEQ ID NO: 8).

In some embodiments, a peptidomimetic macrocycle has the Formula:

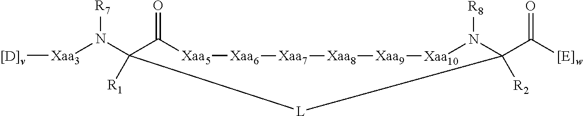

wherein:
each of Xaa₃, Xaa₅, Xaa₆, Xaa₇, Xaa₈, Xaa₉, and Xaa₁₀ is individually an amino acid, wherein at least three of Xaa₃, Xaa₅, Xaa₆, Xaa₇, Xaa₈, Xaa₉, and Xaa₁₀ are the same amino acid as the amino acid at the corresponding position of the sequence Phe₃-X₄-Glu₅-Tyr₆-Trp₇-Ala₈-Gln₉-Leu₁₀/Cba₁₀-X₁₁-Ala₁₂ (SEQ ID NO: 9), wherein each X is an amino acid;
each D is independently a natural or non-natural amino acid or an amino acid analog;
each E is independently a natural or non-natural amino acid or an amino acid analog, for example an amino acid selected from Ala (alanine), D-Ala (D-alanine), Aib (α-aminoisobutyric acid), Sar (N-methyl glycine), and Ser (serine);
R₁ and R₂ are independently —H, alkyl, alkenyl, alkynyl, arylalkyl, cycloalkyl, cycloalkylalkyl, heteroalkyl, or heterocycloalkyl, unsubstituted or substituted with halo-; or at least one of R₁ and R₂ forms a macrocycle-forming linker L′ connected to the alpha position of one of said D or E amino acids;
each L and L′ is independently a macrocycle-forming linker of the formula -L₁-L₂-;
each L₁ and L₂ is independently alkylene, alkenylene, alkynylene, heteroalkylene, cycloalkylene, heterocycloalkylene, arylene, heteroarylene, or [—R₄—K—R₄-]ₙ, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
R₇ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
R₈ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
v is an integer from 1-1000, for example 1-500, 1-200, 1-100, 1-50, 1-30, 1-20, or 1-10;
w is an integer from 3-1000, for example 3-500, 3-200, 3-100, 3-50, 3-30, 3-20, or 3-10; and
n is an integer from 1-5.

In some embodiments of the above Formula, at least three of Xaa₃, Xaa₅, Xaa₆, Xaa₇, Xaa₈, Xaa₉, and Xaa₁₀ are the same amino acid as the amino acid at the corresponding position of the sequence Phe₃-X₄-Glu₅-Tyr₆-Trp₇-Ala₈-Gln₉-Leu₁₀/Cba₁₀-X₁₁-Ala₁₂ (SEQ ID NO: 9). In other embodiments of the above Formula, at least four of Xaa₃, Xaa₅, Xaa₆, Xaa₇, Xaa₈, Xaa₉, and Xaa₁₀ are the same amino acid as the amino acid at the corresponding position of the sequence Phe₃-X₄-Glu₅-Tyr₆-Trp₇-Ala₈-Gln₉-Leu₁₀/Cba₁₀-X₁₁-Ala₁₂ (SEQ ID NO: 9). In other embodiments of the above Formula, at least five of Xaa₃, Xaa₅, Xaa₆, Xaa₇, Xaa₈, Xaa₉, and Xaa₁₀ are the same amino acid as the amino acid at the corresponding position of the sequence Phe₃-X₄-Glu₅-Tyr₆-Trp₇-Ala₈-Gln₉-Leu₁₀/Cba₁₀-X₁₁-Ala₁₂ (SEQ ID NO: 9). In other embodiments of the above Formula, at least six of Xaa₃, Xaa₅, Xaa₆, Xaa₇, Xaa₈, Xaa₉, and Xaa₁₀ are the same amino acid as the amino acid at the corresponding position of the sequence Phe₃-X₄-Glu₅-Tyr₆-Trp₇-Ala₈-Gln₉-Leu₁₀/Cba₁₀-X₁₁-Ala₁₂ (SEQ ID NO: 9). In other embodiments of the above Formula, at least seven of Xaa₃, Xaa₅, Xaa₆, Xaa₇, Xaa₈, Xaa₉, and Xaa₁₀ are the same amino acid as the amino acid at the corresponding position of the sequence Phe₃-X₄-Glu₅-Tyr₆-Trp₇-Ala₈-Gln₉-Leu₁₀/Cba₁₀-X₁₁-Ala₁₂ (SEQ ID NO: 9).

In some embodiments, w is an integer from 3-10, for example 3-6, 3-8, 6-8, or 6-10. In some embodiments, w is 3. In other embodiments, w is 6. In some embodiments, v is an integer from 1-10. In some embodiments, v is 2.

In an embodiment of any of the Formulas described herein, L₁ and L₂, either alone or in combination, do not form a triazole or a thioether.

In one example, at least one of R₁ and R₂ is alkyl, unsubstituted or substituted with halo-. In another example, both R₁ and R₂ are independently alkyl, unsubstituted or substituted with halo-. In some embodiments, at least one of R₁ and R₂ is methyl. In other embodiments, R₁ and R₂ are methyl.

In some embodiments, x+y+z is at least 3. In other embodiments, x+y+z is 1, 2, 3, 4, 5, 6, 7, 8, 9 or 10. In some embodiments, the sum of x+y+z is 3 or 6. In some embodiments, the sum of x+y+z is 3. In other embodiments, the sum of x+y+z is 6. Each occurrence of A, B, C, D or E in a macrocycle or macrocycle precursor is independently selected. For example, a sequence represented by the formula [A]ₓ, when x is 3, encompasses embodiments wherein the amino acids are not identical, e.g. Gln-Asp-Ala as well as embodiments wherein the amino acids are identical, e.g. Gln-Gln-Gln. This applies for any value of x, y, or z in the indicated ranges. Similarly, when u is greater than 1, each compound can encompass peptidomimetic macrocycles which are the same or different. For example, a compound can comprise peptidomimetic macrocycles comprising different linker lengths or chemical compositions.

In some embodiments, the peptidomimetic macrocycle comprises a secondary structure which is an α-helix and R₈ is —H, allowing intra-helical hydrogen bonding. In some embodiments, at least one of A, B, C, D or E is an α,α-disubstituted amino acid. In one example, B is an α,α-disubstituted amino acid. For instance, at least one of A, B, C, D or E is 2-aminoisobutyric acid. In other embodiments, at least one of A, B, C, D or E is

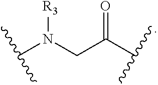

In other embodiments, the length of the macrocycle-forming linker L as measured from a first Cα to a second Cα is selected to stabilize a desired secondary peptide structure, such as an α-helix formed by residues of the peptidomimetic macrocycle including, but not necessarily limited to, those between the first Cα to a second Cα.

In some embodiments, a peptidomimetic macrocycle of Formula (I) has Formula (Ia):

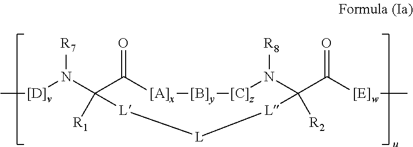

wherein:
each A, C, D, and E is independently a natural or non-natural amino acid or an amino acid analog;
each B is independently a natural or non-natural amino acid, amino acid analog,

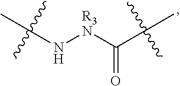

[—NH-L₃-CO—], [—NH-L₃-SO₂—], or [—NH-L₃-];
each L is independently a macrocycle-forming linker;
each L′ is independently alkylene, alkenylene, alkynylene, heteroalkylene, cycloalkylene, heterocycloalkylene, arylene, or heteroarylene, each being optionally substituted with R₅, or a bond, or together with R₁ and the atom to which both R₁ and L′ are bound forms a ring;
each L″ is independently alkylene, alkenylene, alkynylene, heteroalkylene, cycloalkylene, heterocycloalkylene, arylene, or heteroarylene, each being optionally substituted with R₅, or a bond, or together with R₂ and the atom to which both R₂ and L″ are bound forms a ring;
each R₁ is independently —H, alkyl, alkenyl, alkynyl, arylalkyl, cycloalkyl, cycloalkylalkyl, heteroalkyl, or heterocycloalkyl, unsubstituted or substituted with halo-, or together with L′ and the atom to which both R₁ and L′ are bound forms a ring;
each R₂ is independently —H, alkyl, alkenyl, alkynyl, arylalkyl, cycloalkyl, cycloalkylalkyl, heteroalkyl, or heterocycloalkyl, unsubstituted or substituted with halo-, or together with L″ and the atom to which both R₂ and L″ are bound forms a ring;
each R₃ is independently hydrogen, alkyl, alkenyl, alkynyl, arylalkyl, heteroalkyl, cycloalkyl, heterocycloalkyl, cycloalkylalkyl, aryl, or heteroaryl, optionally substituted with R₅;
each L₃ is independently alkylene, alkenylene, alkynylene, heteroalkylene, cycloalkylene, heterocycloalkylene, arylene, heteroarylene, or [—R₄—K—R₄-]ₙ, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
n is an integer from 1-5;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
each R₇ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
each R₈ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
each v and w is independently an integer from 1-1000, for example 1-500, 1-200, 1-100, 1-50, 1-40, 1-25, 1-20, 1-15, or 1-10;
each x, y and z is independently an integer from 0-10, for example x+y+z is 2, 3, or 6; and
u is an integer from 1-10, for example 1-5, 1-3, or 1-2.

In some embodiments, L is a macrocycle-forming linker of the formula -L₁-L₂-. In some embodiments, each L₁ and L₂ is independently alkylene, alkenylene, alkynylene, heteroalkylene, cycloalkylene, heterocycloalkylene, arylene, heteroarylene, or [—R₄—K—R₄-]ₙ, each being optionally substituted with R₅; each R₄ is independently alkylene, alkenylene, alkynylene, heteroalkylene, cycloalkylene, heterocycloalkylene, arylene, or heteroarylene; each K is independently O, S, SO, SO₂, CO, CO₂, or CONR₃; and n is an integer from 1-5.

In one example, at least one of R₁ and R₂ is alkyl, unsubstituted or substituted with halo-. In another example, both R₁ and R₂ are independently alkyl, unsubstituted or substituted with halo-. In some embodiments, at least one of R₁ and R₂ is methyl. In other embodiments, R₁ and R₂ are methyl.

In some embodiments, x+y+z is at least 2. In other embodiments, x+y+z is 1, 2, 3, 4, 5, 6, 7, 8, 9 or 10. Each occurrence of A, B, C, D or E in a macrocycle or macrocycle precursor is independently selected. For example, a sequence represented by the formula [A]ₓ, when x is 3, encompasses embodiments where the amino acids are not identical, e.g. Gln-Asp-Ala as well as embodiments wherein the amino acids are identical, e.g. Gln-Gln-Gln. This applies for any value of x, y, or z in the indicated ranges. Similarly, when u is greater than 1, each compound can encompass peptidomimetic macrocycles which are the same or different. For example, a compound can comprise peptidomimetic macrocycles comprising different linker lengths or chemical compositions.

In some embodiments, the peptidomimetic macrocycle comprises a secondary structure which is a helix and R₈ is —H, allowing intra-helical hydrogen bonding. In some embodiments, at least one of A, B, C, D or E is an α,α-disubstituted amino acid. In one example, B is an α,α-disubstituted amino acid. For instance, at least one of A, B, C, D or E is 2-aminoisobutyric acid. In other embodiments, at least one of A, B, C, D or E is

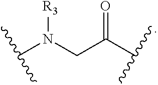

In other embodiments, the length of the macrocycle-forming linker L as measured from a first Cα to a second Cα is selected to stabilize a desired secondary peptide structure, such as a helix formed by residues of the peptidomimetic macrocycle including, but not necessarily limited to, those between the first Cα to a second Cα.

In one embodiment, the peptidomimetic macrocycle of Formula (I) is:

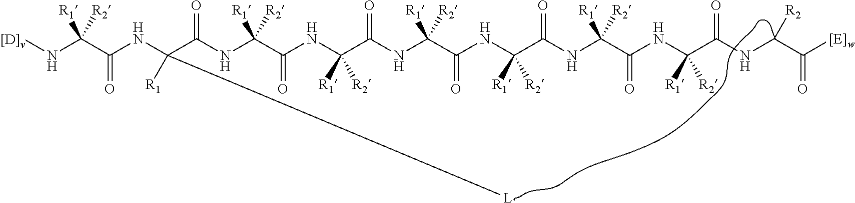

wherein each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-.

In related embodiments, the peptidomimetic macrocycle of Formula (I) is:

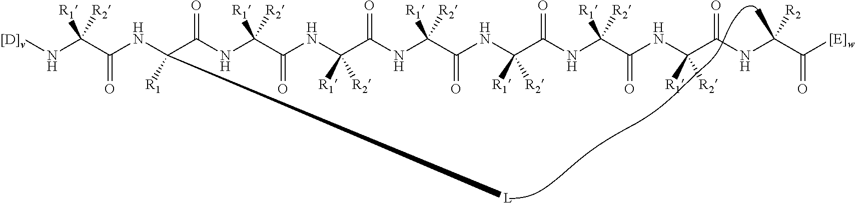

wherein each R₁′ and R₂′ is independently an amino acid.

In other embodiments, the peptidomimetic macrocycle of Formula (I) is a compound of any of the formulas shown below:

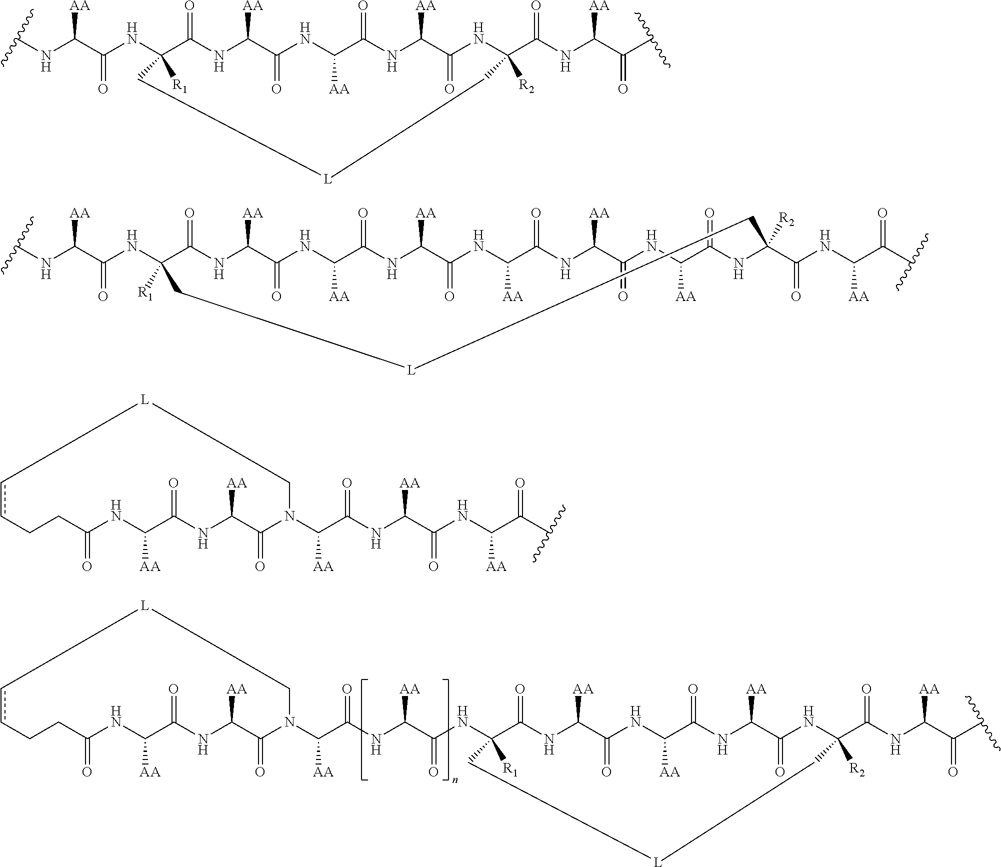

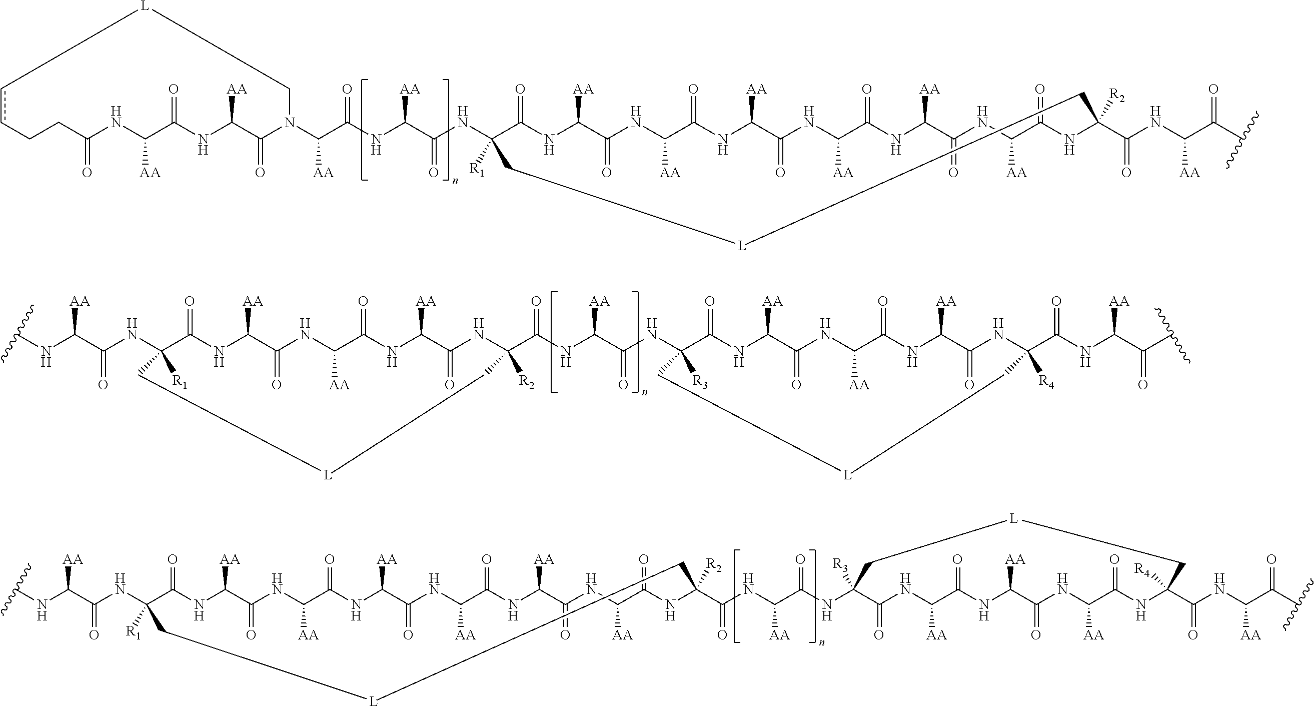

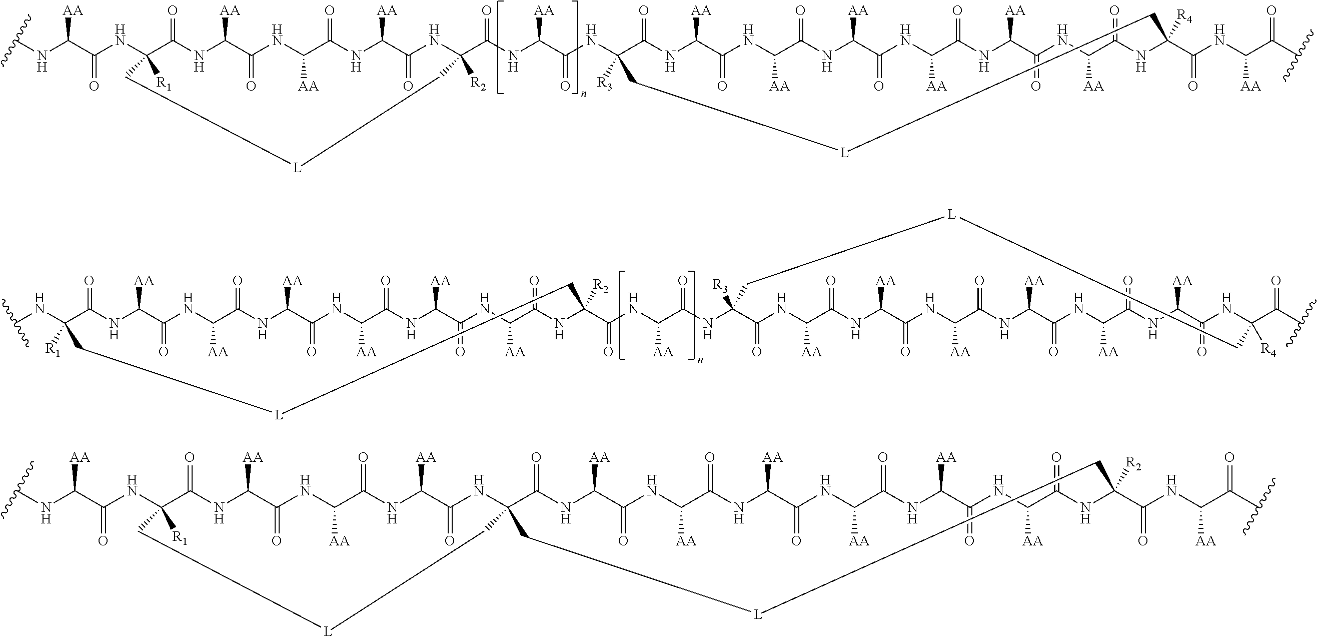

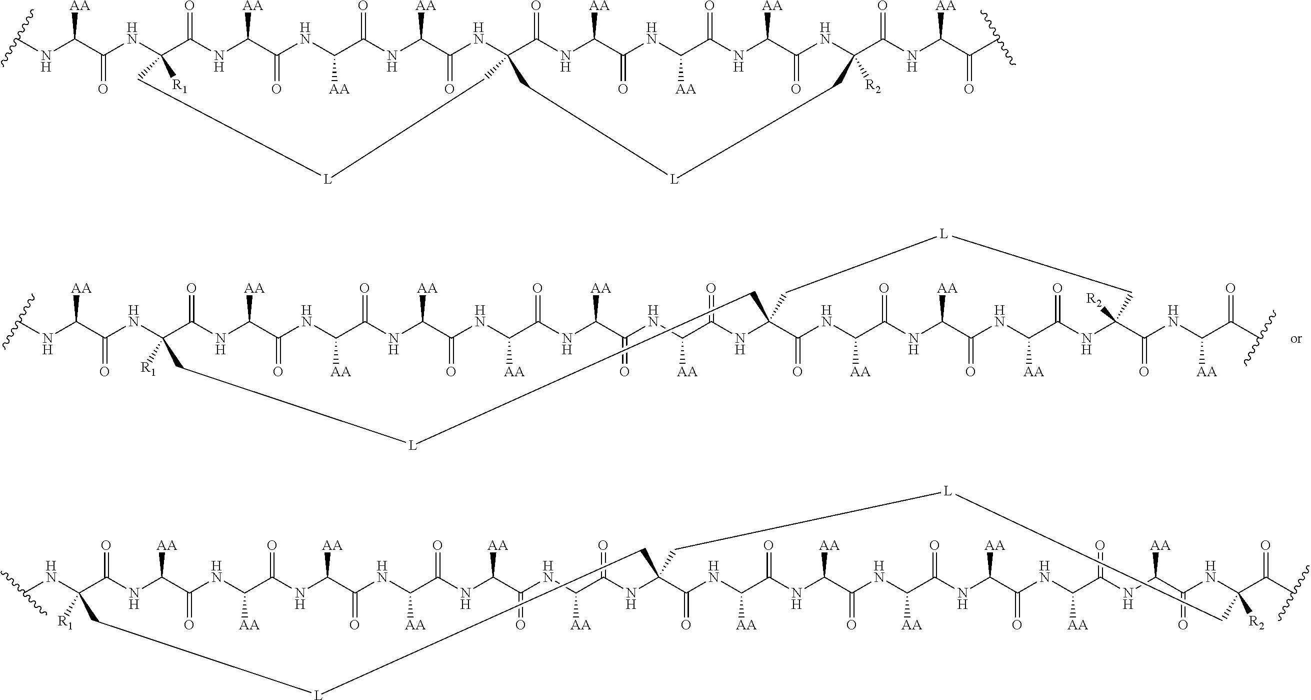

wherein “AA” represents any natural or non-natural amino acid side chain and “ [symbol] ” is [D]ᵥ, [E]_{w} as defined above, and n is an integer between 0 and 20, 50, 100, 200, 300, 400 or 500. In some embodiments, n is 0. In other embodiments, n is less than 50.

Exemplary embodiments of the macrocycle-forming linker L are shown below.

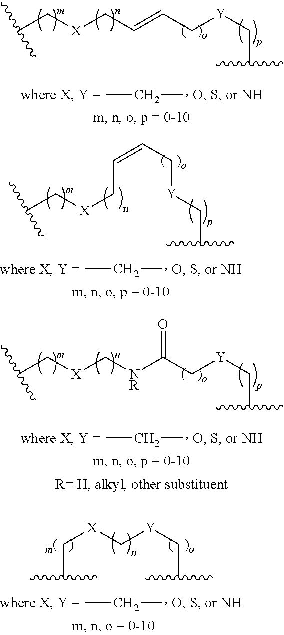

In other embodiments, D and/or E in the compound of Formula I are further modified to facilitate cellular uptake. In some embodiments, lipidating or PEGylating a peptidomimetic macrocycle facilitates cellular uptake, increases bioavailability, increases blood circulation, alters pharmacokinetics, decreases immunogenicity and/or decreases the needed frequency of administration.

In other embodiments, at least one of [D] and [E] in the compound of Formula I represents a moiety comprising an additional macrocycle-forming linker such that the peptidomimetic macrocycle comprises at least two macrocycle-forming linkers. In a specific embodiment, a peptidomimetic macrocycle comprises two macrocycle-forming linkers. In an embodiment, u is 2.

In some embodiments, the peptidomimetic macrocycles have the Formula (I):

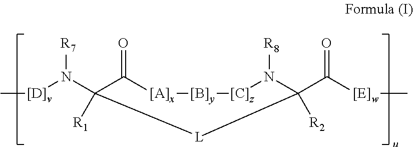

wherein:
each A, C, D, and E is independently a natural or non-natural amino acid or an amino acid analog;
each B is independently a natural or non-natural amino acid, amino acid analog,

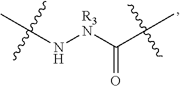

[—NH-L₃-CO—], [—NH-L₃-SO₂—], or [—NH-L₃-];
each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-, or at least one of R₁ and R₂ forms a macrocycle-forming linker L′ connected to the alpha position of one of said D or E amino acids;
each R₃ is independently hydrogen, alkyl, alkenyl, alkynyl, arylalkyl, heteroalkyl, cycloalkyl, heterocycloalkyl, cycloalkylalkyl, aryl, or heteroaryl, optionally substituted with R₅;
each L and L′ is independently macrocycle-forming linker of the formula

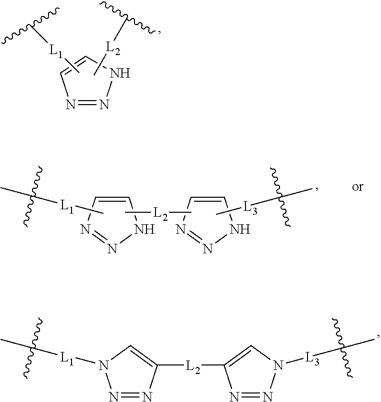

wherein each L₁, L₂ and L₃ is independently alkylene, alkenylene, alkynylene, heteroalkylene, cycloalkylene, heterocycloalkylene, arylene, heteroarylene, or [—R₄—K—R₄-]ₙ, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
each R₇ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
each R₈ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
each v and w is independently an integer from 1-1000;
each x, y and z is independently an integer from 0-10;
us is an integer from 1-10; and
n is an integer from 1-5.

In one example, at least one of R₁ and R₂ is alkyl that is unsubstituted or substituted with halo-. In another example, both R₁ and R₂ are independently alkyl that are unsubstituted or substituted with halo-. In some embodiments, at least one of R₁ and R₂ is methyl. In other embodiments, R₁ and R₂ are methyl.

In some embodiments, x+y+z is at least 2. In other embodiments, x+y+z is 1, 2, 3, 4, 5, 6, 7, 8, 9 or 10. Each occurrence of A, B, C, D or E in a macrocycle or macrocycle precursor is independently selected. For example, a sequence represented by the formula [A]ₓ, when x is 3, encompasses embodiments where the amino acids are not identical, e.g. Gln-Asp-Ala as well as embodiments wherein the amino acids are identical, e.g. Gln-Gln-Gln. This applies for any value of x, y, or z in the indicated ranges.

In some embodiments, each of the first two amino acid represented by E comprises an uncharged side chain or a negatively charged side chain. In some embodiments, each of the first three amino acid represented by E comprises an uncharged side chain or a negatively charged side chain. In some embodiments, each of the first four amino acid represented by E comprises an uncharged side chain or a negatively charged side chain. In some embodiments, one or more or each of the amino acid that is i+1, i+2, i+3, i+4, i+5, and/or i+6 with respect to Xaa₁₃ represented by E comprises an uncharged side chain or a negatively charged side chain.

In some embodiments, the first C-terminal amino acid and/or the second C-terminal amino acid represented by E comprise a hydrophobic side chain. For example, the first C-terminal amino acid and/or the second C-terminal amino acid represented by E comprises a hydrophobic side chain, for example a small hydrophobic side chain. In some embodiments, the first C-terminal amino acid, the second C-terminal amino acid, and/or the third C-terminal amino acid represented by E comprise a hydrophobic side chain. For example, the first C-terminal amino acid, the second C-terminal amino acid, and/or the third C-terminal amino acid represented by E comprises a hydrophobic side chain, for example a small hydrophobic side chain. In some embodiments, one or more or each of the amino acid that is i+1, i+2, i+3, i+4, i+5, and/or i+6 with respect to Xaa₁₃ represented by E comprises an uncharged side chain or a negatively charged side chain.

In some embodiments, w is between 1 and 1000. For example, the first amino acid represented by E comprises a small hydrophobic side chain. In some embodiments, w is between 2 and 1000. For example, the second amino acid represented by E comprises a small hydrophobic side chain. In some embodiments, w is between 3 and 1000. For example, the third amino acid represented by E comprises a small hydrophobic side chain. For example, the third amino acid represented by E comprises a small hydrophobic side chain. In some embodiments, w is between 4 and 1000. In some embodiments, w is between 5 and 1000. In some embodiments, w is between 6 and 1000. In some embodiments, w is between 7 and 1000. In some embodiments, w is between 8 and 1000.

In some embodiments, the peptidomimetic macrocycle comprises a secondary structure which is a helix and R₈ is —H, allowing intra-helical hydrogen bonding. In some embodiments, at least one of A, B, C, D or E is an α,α-disubstituted amino acid. In one example, B is an α,α-disubstituted amino acid. For instance, at least one of A, B, C, D or E is 2-aminoisobutyric acid. In other embodiments, at least one of A, B, C, D or E is

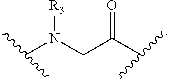

In other embodiments, the length of the macrocycle-forming linker L as measured from a first Cα to a second Cα is selected to stabilize a desired secondary peptide structure, such as a helix formed by residues of the peptidomimetic macrocycle including, but not necessarily limited to, those between the first Cα to a second Cα.

In some embodiments, L is a macrocycle-forming linker of the formula

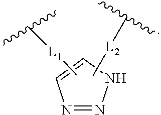

In some embodiments, L is a macrocycle-forming linker of the formula

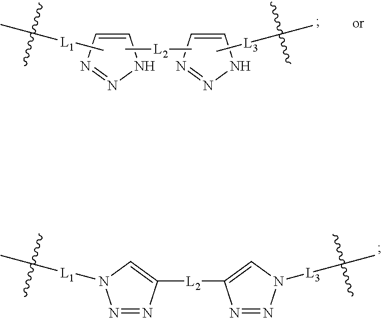

or a tautomer thereof.

Exemplary embodiments of the macrocycle-forming linker L are shown below:

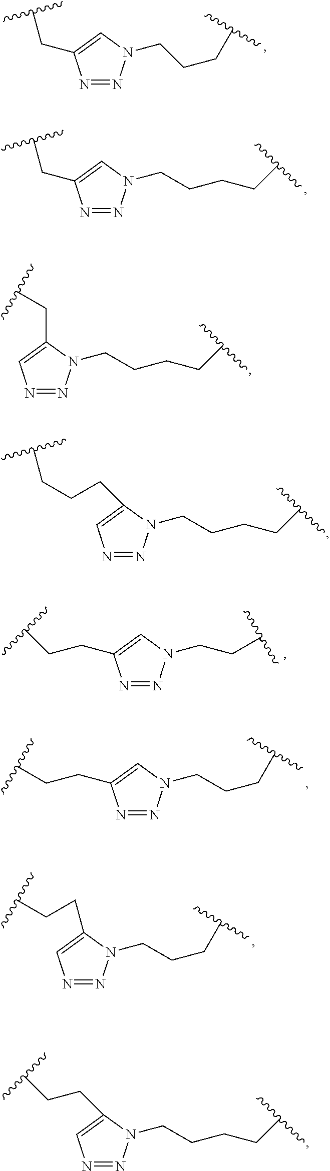

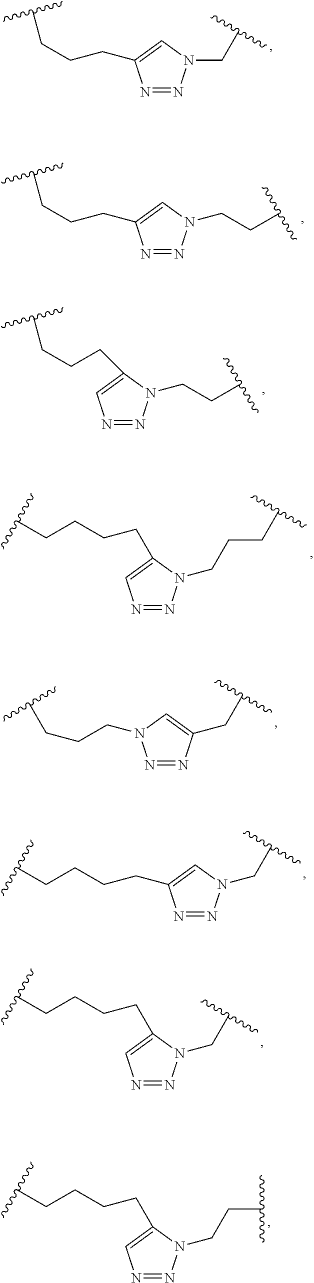

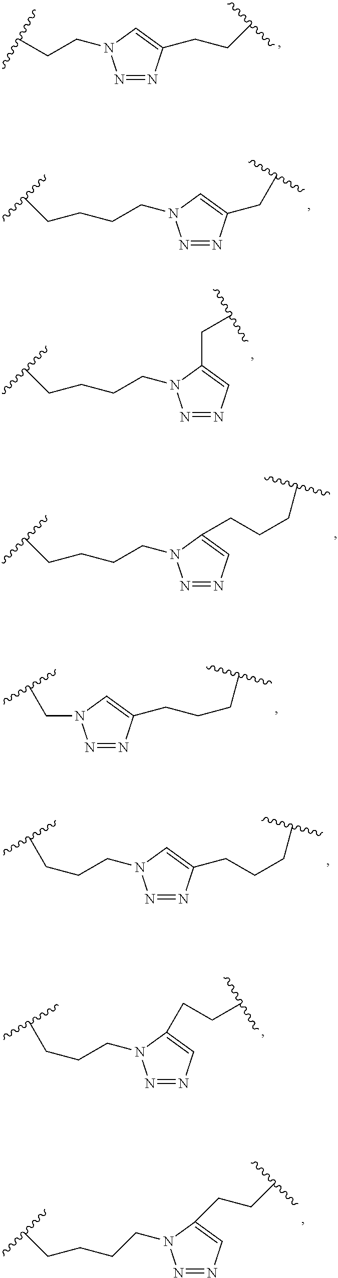

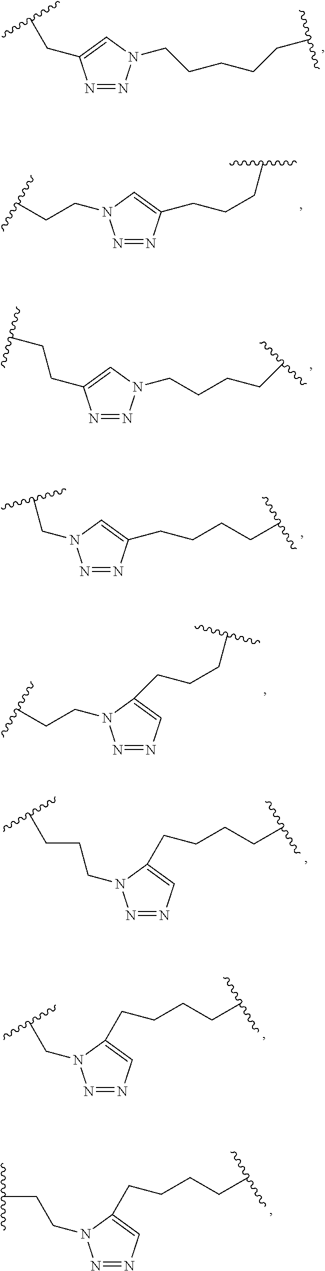

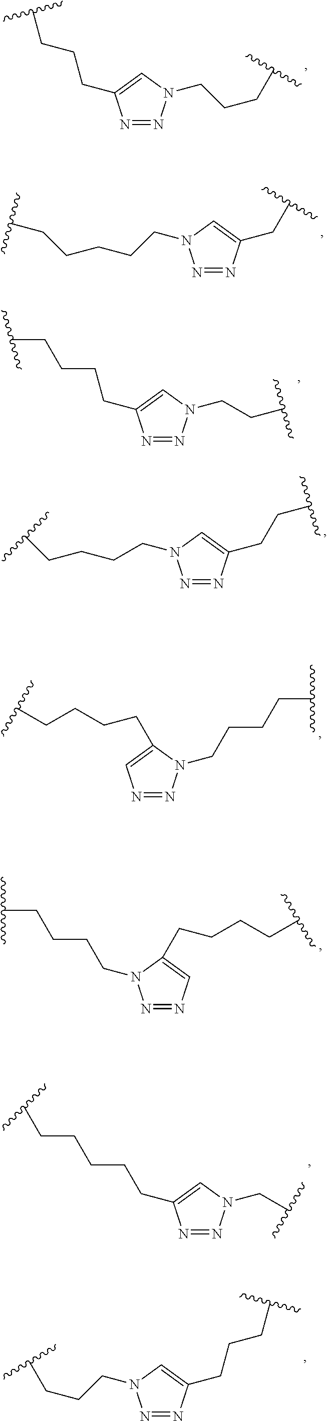

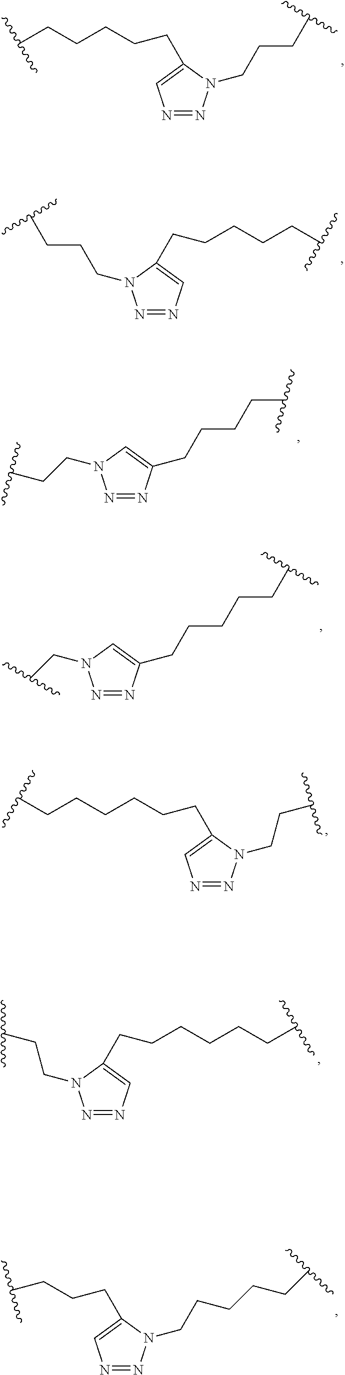

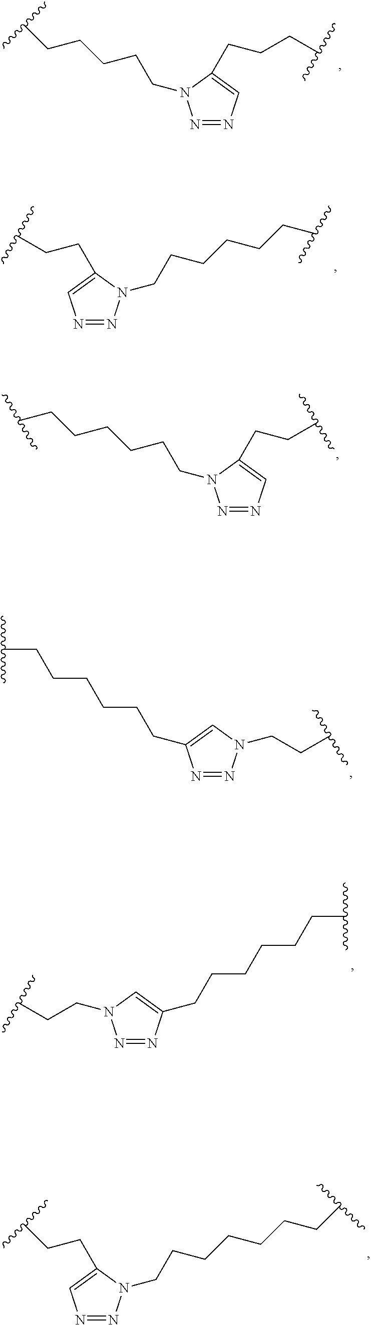 [SMILES: *CCCCCn1nncc1CCC* ; *CCCCCCn1nncc1CC* ; *CCCCCCn1nncc1CC* ; *CCCCCCc1cn(CC*)nn1 ; *CCCCCCc1cn(CC*)nn1 ; *CCCCCCCn1nncc1CC*]

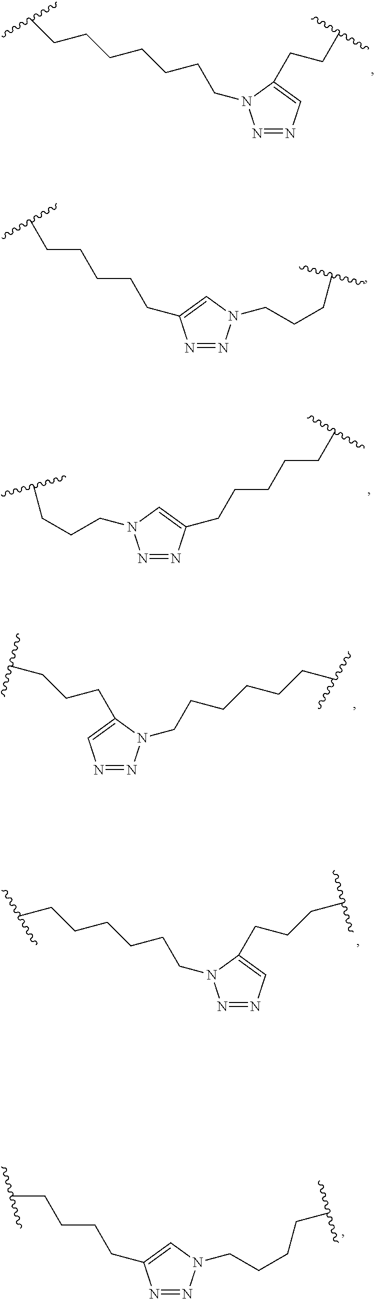 [SMILES: *CCCCCCCn1nncc1CC* ; *CCCCCc1cn(CCC*)nn1 ; *CCCCCc1cn(CCC*)nn1 ; *CCCCCCn1nncc1CCC* ; *CCCCCCn1nncc1CCC* ; *CCCCc1cn(CCCC*)nn1]

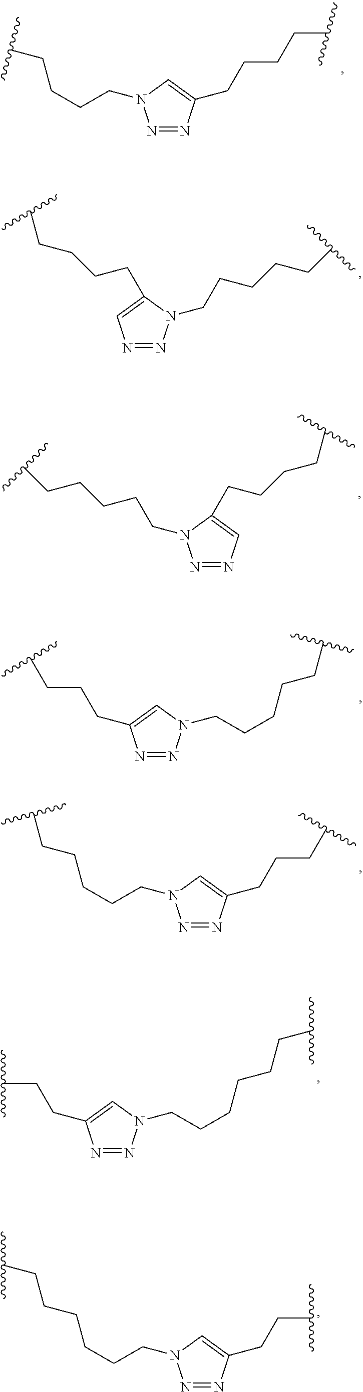

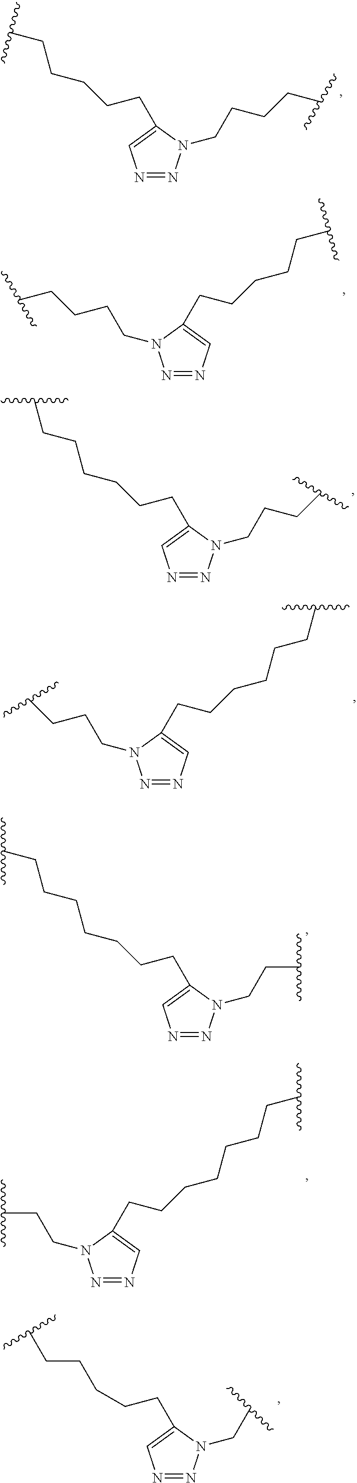

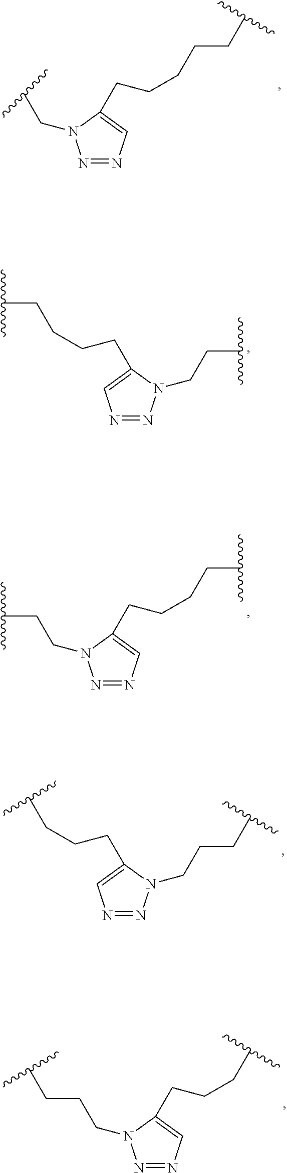 [SMILES: *CCCCCc1cnnn1C* ; *CCCCc1cnnn1CC* ; *CCCCc1cnnn1CC* ; *CCCc1cnnn1CCC* ; *CCCc1cnnn1CCC*]

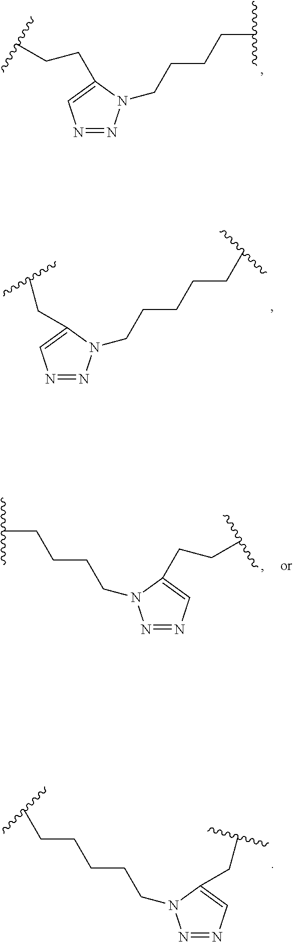 [SMILES: *CCCCn1nncc1CC* ; *CCCCCn1nncc1C* ; *CCCCn1nncc1CC* ; *CCCCCn1nncc1C*]

Amino acids which are used in the formation of triazole crosslinkers are represented according to the legend indicated below. Stereochemistry at the alpha position of each amino acid is S unless otherwise indicated. For azide amino acids, the number of carbon atoms indicated refers to the number of methylene units between the alpha carbon and the terminal azide. For alkyne amino acids, the number of carbon atoms indicated is the number of methylene units between the alpha position and the triazole moiety plus the two carbon atoms within the triazole group derived from the alkyne.
$5a5 Alpha-Me alkyne 1,5 triazole (5 carbon)
$5n3 Alpha-Me azide 1,5 triazole (3 carbon)
$4rn6 Alpha-Me R-azide 1,4 triazole (6 carbon)
$4a5 Alpha-Me alkyne 1,4 triazole (5 carbon)

In some embodiments, any of the macrocycle-forming linkers described herein can be used in any combination with any of the sequences shown in TABLE 1, TABLE 1a, TABLE 1b, or TABLE 1c and also with any of the R-substituents indicated herein.

In some embodiments, the peptidomimetic macrocycle comprises at least one α-helix motif. For example, A, B and/or C in the compound of Formula I include one or more α-helices. As a general matter, α-helices include between 3 and 4 amino acid residues per turn. In some embodiments, the α-helix of the peptidomimetic macrocycle includes 1 to 5 turns and, therefore, 3 to 20 amino acid residues. In specific embodiments, the α-helix includes 1 turn, 2 turns, 3 turns, 4 turns, or 5 turns. In some embodiments, the macrocycle-forming linker stabilizes an α-helix motif included within the peptidomimetic macrocycle. Thus, in some embodiments, the length of the macrocycle-forming linker L from a first Cα to a second Cα is selected to increase the stability of an α-helix.

In some embodiments, the macrocycle-forming linker spans from 1 turn to 5 turns of the α-helix. In some embodiments, the macrocycle-forming linker spans approximately 1 turn, 2 turns, 3 turns, 4 turns, or 5 turns of the α-helix. In some embodiments, the length of the macrocycle-forming linker is approximately 5 Å to 9 Å per turn of the α-helix, or approximately 6 Å to 8 Å per turn of the α-helix.

Where the macrocycle-forming linker spans approximately 1 turn of an α-helix, the length is equal to approximately 5 carbon-carbon bonds to 13 carbon-carbon bonds, approximately 7 carbon-carbon bonds to 11 carbon-carbon bonds, or approximately 9 carbon-carbon bonds. Where the macrocycle-forming linker spans approximately 2 turns of an α-helix, the length is equal to approximately 8 carbon-carbon bonds to 16 carbon-carbon bonds, approximately 10 carbon-carbon bonds to 14 carbon-carbon bonds, or approximately 12 carbon-carbon bonds. Where the macrocycle-forming linker spans approximately 3 turns of an α-helix, the length is equal to approximately 14 carbon-carbon bonds to 22 carbon-carbon bonds, approximately 16 carbon-carbon bonds to 20 carbon-carbon bonds, or approximately 18 carbon-carbon bonds. Where the macrocycle-forming linker spans approximately 4 turns of an α-helix, the length is equal to approximately 20 carbon-carbon bonds to 28 carbon-carbon bonds, approximately 22 carbon-carbon bonds to 26 carbon-carbon bonds, or approximately 24 carbon-carbon bonds. Where the macrocycle-forming linker spans approximately 5 turns of an α-helix, the length is equal to approximately 26 carbon-carbon bonds to 34 carbon-carbon bonds, approximately 28 carbon-carbon bonds to 32 carbon-carbon bonds, or approximately 30 carbon-carbon bonds. Where the macrocycle-forming linker spans approximately 1 turn of an α-helix, the linkage contains approximately 4 atoms to 12 atoms, approximately 6 atoms to 10 atoms, or approximately 8 atoms. Where the macrocycle-forming linker spans approximately 2 turns of the α-helix, the linkage contains approximately 7 atoms to 15 atoms, approximately 9 atoms to 13 atoms, or approximately 11 atoms. Where the macrocycle-forming linker spans approximately 3 turns of the α-helix, the linkage contains approximately 13 atoms to 21 atoms, approximately 15 atoms to 19 atoms, or approximately 17 atoms. Where the macrocycle-forming linker spans approximately 4 turns of the α-helix, the linkage contains approximately 19 atoms to 27 atoms, approximately 21 atoms to 25 atoms, or approximately 23 atoms. Where the macrocycle-forming linker spans approximately 5 turns of the α-helix, the linkage contains approximately 25 atoms to 33 atoms, approximately 27 atoms to 31 atoms, or approximately 29 atoms.

Where the macrocycle-forming linker spans approximately 1 turn of the α-helix, the resulting macrocycle forms a ring containing approximately 17 members to 25 members, approximately 19 members to 23 members, or approximately 21 members. Where the macrocycle-forming linker spans approximately 2 turns of the α-helix, the resulting macrocycle forms a ring containing approximately 29 members to 37 members, approximately 31 members to 35 members, or approximately 33 members. Where the macrocycle-forming linker spans approximately 3 turns of the α-helix, the resulting macrocycle forms a ring containing approximately 44 members to 52 members, approximately 46 members to 50 members, or approximately 48 members. Where the macrocycle-forming linker spans approximately 4 turns of the α-helix, the resulting macrocycle forms a ring containing approximately 59 members to 67 members, approximately 61 members to 65 members, or approximately 63 members. Where the macrocycle-forming linker spans approximately 5 turns of the α-helix, the resulting macrocycle forms a ring containing approximately 74 members to 82 members, approximately 76 members to 80 members, or approximately 78 members.

In other embodiments, provided are peptidomimetic macrocycles of Formula (II) or (IIa):

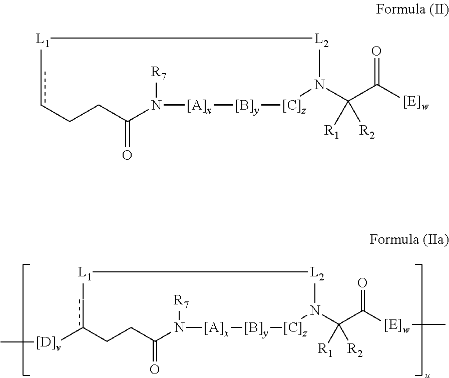

wherein:
each A, C, D, and E is independently a natural or non-natural amino acid or an amino acid analog, and the terminal D and E independently optionally include a capping group;
each B is independently a natural or non-natural amino acid, amino acid analog,

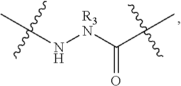

[—NH-L₃-CO—], [—NH-L₃-SO₂—], or [—NH-L₃-];
each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-; or at least one of R₁ and R₂ forms a macrocycle-forming linker L′ connected to the alpha position of one of said D or E amino acids;
each R₃ is independently hydrogen, alkyl, alkenyl, alkynyl, arylalkyl, heteroalkyl, cycloalkyl, heterocycloalkyl, cycloalkylalkyl, aryl, or heteroaryl, optionally substituted with R₅;
each L and L′ is a macrocycle-forming linker of the formula -L₁-L₂-;
each L₁, L₂, and L₃ is independently alkylene, alkenylene, alkynylene, heteroalkylene, cycloalkylene, heterocycloalkylene, arylene, heteroarylene, or [—R₄—K—R₄-]ₙ, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
each R₇ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅;
each v and w is independently an integer from 1-1000;
u is an integer from 1-10;
each x, y, and z is independently integers from 0-10; and
n is an integer from 1-5.

In one example, L₁ and L₂, either alone or in combination, do not form a triazole or a thioether.

In one example, at least one of R₁ and R₂ is alkyl, unsubstituted or substituted with halo-. In another example, both R₁ and R₂ are independently alkyl, unsubstituted or substituted with halo-. In some embodiments, at least one of R₁ and R₂ is methyl. In other embodiments, R₁ and R₂ are methyl.

In some embodiments, x+y+z is at least 1. In other embodiments, x+y+z is at least 2. In other embodiments, x+y+z is 1, 2, 3, 4, 5, 6, 7, 8, 9 or 10. Each occurrence of A, B, C, D or E in a macrocycle or macrocycle precursor is independently selected. For example, a sequence represented by the formula [A]ₓ, when x is 3, encompasses embodiments wherein the amino acids are not identical, e.g. Gln-Asp-Ala as well as embodiments wherein the amino acids are identical, e.g. Gln-Gln-Gln. This applies for any value of x, y, or z in the indicated ranges.

In some embodiments, the peptidomimetic macrocycle comprises a secondary structure which is an α-helix and R₈ is —H, allowing intra-helical hydrogen bonding. In some embodiments, at least one of A, B, C, D or E is an α,α-disubstituted amino acid. In one example, B is an α,α-disubstituted amino acid. For example, at least one of A, B, C, D or E is 2-aminoisobutyric acid. In other embodiments, at least one of A, B, C, D or E is

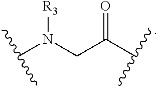

In other embodiments, the length of the macrocycle-forming linker L as measured from a first Cα to a second Cα is selected to stabilize a desired secondary peptide structure, such as an α-helix formed by residues of the peptidomimetic macrocycle including, but not necessarily limited to, those between the first Cα to a second Cα.

Exemplary embodiments of the macrocycle-forming linker -L₁-L₂- are shown below.

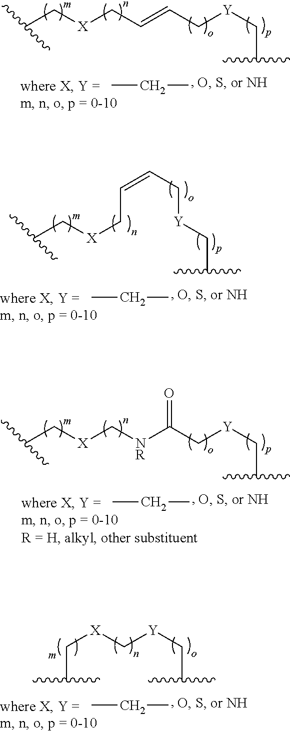

In some embodiments, the peptidomimetic macrocycle has the Formula (III) or Formula (IIIa):

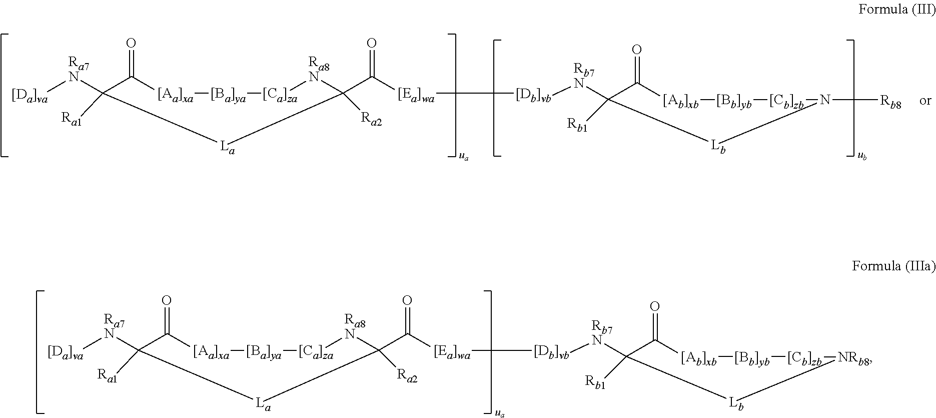

wherein:
each Aₐ, Cₐ, Dₐ, Eₐ, A_{b}, C_{b}, and D_{b} is independently a natural or non-natural amino acid or an amino acid analog;
each Bₐ and B_{b} is independently a natural or non-natural amino acid, amino acid analog,

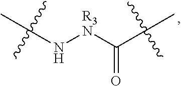

[—NH-L₄-CO—], [—NH-L₄-SO₂—], or [—NH-L₄-];
each Rₐ₁ is independently alkyl, alkenyl, alkynyl, arylalkyl, cycloalkyl, cycloalkylalkyl, heteroalkyl, or heterocycloalkyl, any of which is unsubstituted or substituted; or H; or Rₐ₁ forms a macrocycle-forming linker L′ connected to the alpha position of one of the Dₐ or Eₐ amino acids; or together with Lₐ forms a ring that is unsubstituted or substituted;
each Rₐ₂ is independently alkyl, alkenyl, alkynyl, arylalkyl, cycloalkyl, cycloalkylalkyl, heteroalkyl, or heterocycloalkyl, any of which is unsubstituted or substituted; or H; or Rₐ₂ forms a macrocycle-forming linker L′ connected to the alpha position of one of the Dₐ or Eₐ amino acids; or together with Lₐ forms a ring that is unsubstituted or substituted;
each R_{b1} is independently alkyl, alkenyl, alkynyl, arylalkyl, cycloalkyl, cycloalkylalkyl, heteroalkyl, or heterocycloalkyl, any of which is unsubstituted or substituted; or H; or R_{b1} forms a macrocycle-forming linker L′ connected to the alpha position of one of the D_{b} amino acids; or together with L_{b} forms a ring that is unsubstituted or substituted;
each R₃ is independently alkyl, alkenyl, alkynyl, arylalkyl, heteroalkyl, cycloalkyl, heterocycloalkyl, cycloalkylalkyl, cycloaryl, or heterocycloaryl, any of which is unsubstituted or substituted, or H;
each Lₐ is independently a macrocycle-forming linker, and optionally forms a ring with Rₐ₁ or Rₐ₂ that is unsubstituted or substituted;
each L_{b} is independently a macrocycle-forming linker, and optionally forms a ring with R_{b1} that is unsubstituted or substituted;
each L′ is independently a macrocycle-forming linker;
each L₄ is independently alkylene, alkenylene, alkynylene, heteroalkylene, cycloalkylene, heterocycloalkylene, cycloarylene, heterocycloarylene, or [—R₄—K—R₄-]ₙ, any of which is unsubstituted or substituted;
each R₄ is independently alkylene, alkenylene, alkynylene, heteroalkylene, cycloalkylene, heterocycloalkylene, arylene, or heteroarylene, any of which is unsubstituted or substituted;
each K is independently O, S, SO, SO₂, CO, CO₂, OCO₂, NR₃, CONR₃, OCONR₃, OSO₂NR₃, NR_{3q}, CONR_{3q}, OCONR_{3q}, or OSO₂NR_{3q}, wherein each R_{3q} is independently a point of attachment to Rₐ₁, Rₐ₂, or R_{b1};
Rₐ₇ is alkyl, alkenyl, alkynyl, arylalkyl, cycloalkyl, heteroalkyl, cycloalkylalkyl, heterocycloalkyl, cycloaryl, or heterocycloaryl, any of which is unsubstituted or substituted; or H; or part of a cyclic structure with a Dₐ amino acid;
R_{b7} is alkyl, alkenyl, alkynyl, arylalkyl, cycloalkyl, heteroalkyl, cycloalkylalkyl, heterocycloalkyl, cycloaryl, or heterocycloaryl, any of which is unsubstituted or substituted; or H; or part of a cyclic structure with a D_{b} amino acid;
Rₐ₈ is alkyl, alkenyl, alkynyl, arylalkyl, cycloalkyl, heteroalkyl, cycloalkylalkyl, heterocycloalkyl, cycloaryl, or heterocycloaryl, any of which is unsubstituted or substituted; or H; or part of a cyclic structure with an Eₐ amino acid;
R_{b8} is alkyl, alkenyl, alkynyl, arylalkyl, cycloalkyl, heteroalkyl, cycloalkylalkyl, heterocycloalkyl, cycloaryl, or heterocycloaryl, any of which is unsubstituted or substituted; or H; or an amino acid sequence of 1-1000 amino acid residues;
each va and vb is independently an integer from 0-1000;
each wa and wb is independently an integer from 0-1000;
each ua and ub is independently 0, 1, 2, 3, 4, 5, 6, 7, 8, 9, or 10, wherein ua+ub is at least 1;
each xa and xb is independently 0, 1, 2, 3, 4, 5, 6, 7, 8, 9, or 10;
each ya and yb is independently 0, 1, 2, 3, 4, 5, 6, 7, 8, 9, or 10;
each za and zb is independently 0, 1, 2, 3, 4, 5, 6, 7, 8, 9, or 10; and
each n is independently 1, 2, 3, 4, or 5,
   or a pharmaceutically-acceptable salt thereof.

In some embodiments, the peptidomimetic macrocycle has the Formula (III) or Formula (IIIa):

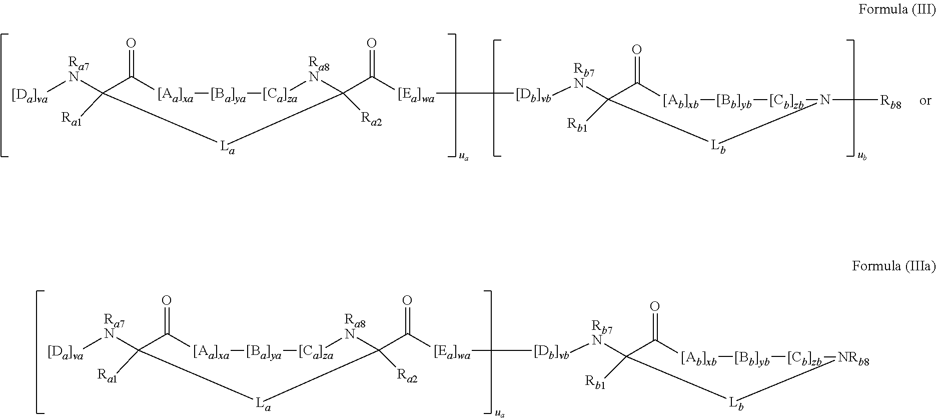

wherein:
each Aₐ, Cₐ, Dₐ, Eₐ, A_{b}, C_{b}, and D_{b} is independently a natural or non-natural amino acid or an amino acid analogue;
each Bₐ and B_{b} is independently a natural or non-natural amino acid, amino acid analog,

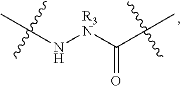

[—NH-L₄-CO—], [—NH-L₄-SO₂—], or [—NH-L₄-];
each Rₐ₁ is independently alkyl, alkenyl, alkynyl, arylalkyl, cycloalkyl, cycloalkylalkyl, heteroalkyl, or heterocycloalkyl, any of which is unsubstituted or substituted; or H; or Rₐ₁ forms a macrocycle-forming linker L′ connected to the alpha position of one of the Dₐ or Eₐ amino acids; or together with Lₐ forms a ring that is unsubstituted or substituted;
each Rₐ₂ is independently alkyl, alkenyl, alkynyl, arylalkyl, cycloalkyl, cycloalkylalkyl, heteroalkyl, or heterocycloalkyl, any of which is unsubstituted or substituted; or H; or Rₐ₂ forms a macrocycle-forming linker L′ connected to the alpha position of one of the Dₐ or Eₐ amino acids; or together with Lₐ forms a ring that is unsubstituted or substituted;
each R_{b1} is independently alkyl, alkenyl, alkynyl, arylalkyl, cycloalkyl, cycloalkylalkyl, heteroalkyl, or heterocycloalkyl, any of which is unsubstituted or substituted; or H; or R_{b1} forms a macrocycle-forming linker L′ connected to the alpha position of one of the D_{b} amino acids; or together with L_{b} forms a ring that is unsubstituted or substituted;
each R₃ is independently alkyl, alkenyl, alkynyl, arylalkyl, heteroalkyl, cycloalkyl, heterocycloalkyl, cycloalkylalkyl, cycloaryl, or heterocycloaryl, any of which is unsubstituted or substituted with R₅, or H;
each Lₐ is independently a macrocycle-forming linker, and optionally forms a ring with Rₐ₁ or Rₐ₂ that is unsubstituted or substituted;
each L_{b} is independently a macrocycle-forming linker, and optionally forms a ring with R_{b1} that is unsubstituted or substituted;
each L′ is independently a macrocycle-forming linker;
each L₄ is independently alkylene, alkenylene, alkynylene, heteroalkylene, cycloalkylene, heterocycloalkylene, cycloarylene, heterocycloarylene, or [—R₄—K—R₄-]ₙ, any of which is unsubstituted or substituted with R₅;
each R₄ is independently alkylene, alkenylene, alkynylene, heteroalkylene, cycloalkylene, heterocycloalkylene, arylene, or heteroarylene, any of which is unsubstituted or substituted with R₅;
each K is independently O, S, SO, SO₂, CO, CO₂, OCO₂, NR₃, CONR₃, OCONR₃, OSO₂NR₃, NR_{3q}, CONR_{3q}, OCONR_{3q}, or OSO₂NR_{3q}, wherein each R_{3q} is independently a point of attachment to Rₐ₁, Rₐ₂, or R_{b1};
each R₅ is independently halogen, alkyl, —OR₆, —N(R₆)₂, —SR₆, —SOR₆, —SO₂R₆, —CO₂R₆, a fluorescent moiety, a radioisotope, or a therapeutic agent;
each R₆ is independently H, alkyl, alkenyl, alkynyl, arylalkyl, cycloalkylalkyl, heterocycloalkyl, a fluorescent moiety, a radioisotope or a therapeutic agent;
each Rₐ is independently alkyl, alkenyl, alkynyl, arylalkyl, cycloalkyl, heteroalkyl, cycloalkylalkyl, heterocycloalkyl, cycloaryl, or heterocycloaryl, any of which is unsubstituted or substituted with R₅; or H; or part of a cyclic structure with a Dₐ amino acid;
R_{b7} is alkyl, alkenyl, alkynyl, arylalkyl, cycloalkyl, heteroalkyl, cycloalkylalkyl, heterocycloalkyl, cycloaryl, or heterocycloaryl, any of which is unsubstituted or substituted with R₅; or H; or part of a cyclic structure with a D_{b} amino acid;
each Rₐ₈ is independently alkyl, alkenyl, alkynyl, arylalkyl, cycloalkyl, heteroalkyl, cycloalkylalkyl, heterocycloalkyl, cycloaryl, or heterocycloaryl, any of which is unsubstituted or substituted with R₅; or H; or part of a cyclic structure with an Eₐ amino acid;
R_{b8} is alkyl, alkenyl, alkynyl, arylalkyl, cycloalkyl, heteroalkyl, cycloalkylalkyl, heterocycloalkyl, cycloaryl, or heterocycloaryl, any of which is unsubstituted or substituted with R₅; or H; or an amino acid sequence of 1-1000 amino acid residues;
each va and vb is independently an integer from 0-1000;
each wa and wb is independently an integer from 0-1000;
each ua and ub is independently 0, 1, 2, 3, 4, 5, 6, 7, 8, 9, or 10, wherein ua+ub is at least 1;
each xa and xb is independently 0, 1, 2, 3, 4, 5, 6, 7, 8, 9, or 10;
each ya and yb is independently 0, 1, 2, 3, 4, 5, 6, 7, 8, 9, or 10;
each za and zb is independently 0, 1, 2, 3, 4, 5, 6, 7, 8, 9, or 10; and
each n is independently 1, 2, 3, 4, or 5,
   or a pharmaceutically-acceptable salt thereof.

In some embodiments, the peptidomimetic macrocycle of the invention has the formula defined above, wherein:
each Lₐ is independently a macrocycle-forming linker of the formula -L₁-L₂-, and optionally forms a ring with Rₐ₁ or Rₐ₂ that is unsubstituted or substituted;
each L_{b} is independently a macrocycle-forming linker of the formula -L₁-L₂-, and optionally forms a ring with R_{b1} that is unsubstituted or substituted;
each L′ is independently a macrocycle-forming linker of the formula -L₁-L₂-;
each L₁ and L₂ is independently alkylene, alkenylene, alkynylene, heteroalkylene, cycloalkylene, heterocycloalkylene, cycloarylene, heterocycloarylene, or [—R₄—K—R₄-]ₙ, any of which is unsubstituted or substituted with R₅;
each R₄ is independently alkylene, alkenylene, alkynylene, heteroalkylene, cycloalkylene, heterocycloalkylene, arylene, or heteroarylene, any of which is unsubstituted or substituted with R₅;
each K is independently O, S, SO, SO₂, CO, CO₂, OCO₂, NR₃, CONR₃, OCONR₃, OSO₂NR₃, NR_{3q}, CONR_{3q}, OCONR_{3q}, or OSO₂NR_{3q}, wherein each R_{3q} is independently a point of attachment to Rₐ₁, Rₐ₂, or R_{b1};
each R₅ is independently halogen, alkyl, —OR₆, —N(R₆)₂, —SR₆, —SOR₆, —SO₂R₆, —CO₂R₆, a fluorescent moiety, a radioisotope, or a therapeutic agent; and
each R₆ is independently H, alkyl, alkenyl, alkynyl, arylalkyl, cycloalkylalkyl, heterocycloalkyl, a fluorescent moiety, a radioisotope or a therapeutic agent,
   or a pharmaceutically-acceptable salt thereof.

In some embodiments, the peptidomimetic macrocycle has the formula defined above wherein one of Lₐ and L_{b} is a bis-thioether-containing macrocycle-forming linker. In some embodiments, one of Lₐ and L_{b} is a macrocycle-forming linker of the formula -L₁-S-L₂-S-L₃-.

In some embodiments, the peptidomimetic macrocycle has the formula defined above wherein one of Lₐ and L_{b} is a bis-sulfone-containing macrocycle-forming linker. In some embodiments, one of Lₐ and L_{b} is a macrocycle-forming linker of the formula -L₁-SO₂-L₂-SO₂-L₃-.

In some embodiments, the peptidomimetic macrocycle has the formula defined above wherein one of Lₐ and L_{b} is a bis-sulfoxide-containing macrocycle-forming linker. In some embodiments, one of Lₐ and L_{b} is a macrocycle-forming linker of the formula -L₁-S(O)-L₂-S(O)-L₃-.

In some embodiments, a peptidomimetic macrocycle of the invention comprises one or more secondary structures. In some embodiments, the peptidomimetic macrocycle comprises a secondary structure that is an α-helix. In some embodiments, the peptidomimetic macrocycle comprises a secondary structure that is a β-hairpin turn.

In some embodiments, uₐ is 0. In some embodiments, uₐ is 0, and L_{b} is a macrocycle-forming linker that crosslinks an α-helical secondary structure. In some embodiments, uₐ is 0, and L_{b} is a macrocycle-forming linker that crosslinks a β-hairpin secondary structure. In some embodiments, uₐ is 0, and L_{b} is a hydrocarbon-containing macrocycle-forming linker that crosslinks an α-helical secondary structure. In some embodiments, uₐ is 0, and L_{b} is a hydrocarbon-containing macrocycle-forming linker that crosslinks a β-hairpin secondary structure.

In some embodiments, u_{b} is 0. In some embodiments, u_{b} is 0, and Lₐ is a macrocycle-forming linker that crosslinks an α-helical secondary structure. In some embodiments, u_{b} is 0, and Lₐ is a macrocycle-forming linker that crosslinks a 3-hairpin secondary structure. In some embodiments, u_{b} is 0, and Lₐ is a hydrocarbon-containing macrocycle-forming linker that crosslinks an α-helical secondary structure. In some embodiments, u_{b} is 0, and Lₐ is a hydrocarbon-containing macrocycle-forming linker that crosslinks a 3-hairpin secondary structure.

In some embodiments, the peptidomimetic macrocycle comprises only α-helical secondary structures. In other embodiments, the peptidomimetic macrocycle comprises only β-hairpin secondary structures.

In other embodiments, the peptidomimetic macrocycle comprises a combination of secondary structures, wherein the secondary structures are α-helical and β-hairpin structures. In some embodiments, Lₐ and L_{b} are a combination of hydrocarbon-, triazole, or sulfur-containing macrocycle-forming linkers. In some embodiments, the peptidomimetic macrocycle comprises Lₐ and L_{b}, wherein Lₐ is a hydrocarbon-containing macrocycle-forming linker that crosslinks a β-hairpin structure, and L_{b} is a triazole-containing macrocycle-forming linker that crosslinks an α-helical structure. In some embodiments, the peptidomimetic macrocycle comprises Lₐ and L_{b}, wherein Lₐ is a hydrocarbon-containing macrocycle-forming linker that crosslinks an α-helical structure, and L_{b} is a triazole-containing macrocycle-forming linker that crosslinks a β-hairpin structure. In some embodiments, the peptidomimetic macrocycle comprises Lₐ and L_{b}, wherein Lₐ is a triazole-containing macrocycle-forming linker that crosslinks an α-helical structure, and L_{b} is a hydrocarbon-containing macrocycle-forming linker that crosslinks a β-hairpin structure. In some embodiments, the peptidomimetic macrocycle comprises Lₐ and L_{b}, wherein Lₐ is a triazole-containing macrocycle-forming linker that crosslinks a β-hairpin structure, and L_{b} is a hydrocarbon-containing macrocycle-forming linker that crosslinks an α-helical structure.

In some embodiments, uₐ+u_{b} is at least 1. In some embodiments, uₐ+u_{b}=2.

In some embodiments, uₐ is 1, u_{b} is 1, Lₐ is a triazole-containing macrocycle-forming linker that crosslinks an α-helical secondary structure, and L_{b} is a hydrocarbon-containing macrocycle-forming linker that crosslinks an α-helical structure. In some embodiments, uₐ is 1, u_{b} is 1, Lₐ is a triazole-containing macrocycle-forming linker that crosslinks an α-helical secondary structure, and L_{b} is a hydrocarbon-containing macrocycle-forming linker that crosslinks a β-hairpin structure. In some embodiments, uₐ is 1, u_{b} is 1, Lₐ is a triazole-containing macrocycle-forming linker that crosslinks a β-hairpin secondary structure, and L_{b} is a hydrocarbon-containing macrocycle-forming linker that crosslinks an α-helical structure. In some embodiments, uₐ is 1, u_{b} is 1, Lₐ is a triazole-containing macrocycle-forming linker that crosslinks a β-hairpin secondary structure, and L_{b} is a hydrocarbon-containing macrocycle-forming linker that crosslinks a β-hairpin structure.

In some embodiments, uₐ is 1, u_{b} is 1, Lₐ is a hydrocarbon-containing macrocycle-forming linker that crosslinks an α-helical secondary structure, and L_{b} is a triazole-containing macrocycle-forming linker that crosslinks an α-helical secondary structure. In some embodiments, uₐ is 1, u_{b} is 1, Lₐ is a hydrocarbon-containing macrocycle-forming linker that crosslinks an α-helical secondary structure, and L_{b} is a triazole-containing macrocycle-forming linker that crosslinks a β-hairpin secondary structure. In some embodiments, uₐ is 1, u_{b} is 1, Lₐ is a hydrocarbon-containing macrocycle-forming linker that crosslinks a β-hairpin secondary structure, and L_{b} is a triazole-containing macrocycle-forming linker that crosslinks an α-helical secondary structure. In some embodiments, uₐ is 1, u_{b} is 1, Lₐ is a hydrocarbon-containing macrocycle-forming linker that crosslinks a β-hairpin secondary structure, and L_{b} is a triazole-containing macrocycle-forming linker that crosslinks a β-hairpin secondary structure.

In some embodiments, uₐ is 1, u_{b} is 1, Lₐ is a hydrocarbon-containing macrocycle-forming linker with an α-helical secondary structure, and L_{b} is a sulfur-containing macrocycle-forming linker. In some embodiments, uₐ is 1, u_{b} is 1, Lₐ is a hydrocarbon-containing macrocycle-forming linker with a β-hairpin secondary structure, and L_{b} is a sulfur-containing macrocycle-forming linker.

In some embodiments, uₐ is 1, u_{b} is 1, Lₐ is a sulfur-containing macrocycle-forming linker, and L_{b} is a hydrocarbon-containing macrocycle-forming linker with an α-helical secondary structure. In some embodiments, uₐ is 1, u_{b} is 1, Lₐ is a sulfur-containing macrocycle-forming linker, and L_{b} is a hydrocarbon-containing macrocycle-forming linker with a β-hairpin secondary structure.

In some embodiments, uₐ is 1, u_{b} is 1, Lₐ is a hydrocarbon-containing macrocycle-forming linker that crosslinks an α-helical structure, and L_{b} is a hydrocarbon-containing macrocycle-forming linker that crosslinks an α-helical structure. In some embodiments, uₐ is 1, u_{b} is 1, Lₐ is a hydrocarbon-containing macrocycle-forming linker that crosslinks an α-helical structure, and L_{b} is a hydrocarbon-containing macrocycle-forming linker that crosslinks a β-hairpin structure. In some embodiments, uₐ is 1, u_{b} is 1, Lₐ is a hydrocarbon-containing macrocycle-forming linker that crosslinks a β-hairpin structure, and L_{b} is a hydrocarbon-containing macrocycle-forming linker that crosslinks an α-helical structure. In some embodiments, uₐ is 1, u_{b} is 1, Lₐ is a hydrocarbon-containing macrocycle-forming linker that crosslinks a β-hairpin structure, and L_{b} is a hydrocarbon-containing macrocycle-forming linker that crosslinks a β-hairpin structure.

In some embodiments, R_{b1} is H.

Unless otherwise stated, any compounds (including peptidomimetic macrocycles, macrocycle precursors, and other compositions) are also meant to encompass compounds which differ only in the presence of one or more isotopically enriched atoms. For example, compounds having the described structures except for the replacement of a hydrogen atom by deuterium or tritium, or the replacement of a carbon atom by ¹³C or ¹⁴C are contemplated.

In some embodiments, the compounds disclosed herein can contain unnatural proportions of atomic isotopes at one or more of atoms that constitute such compounds. For example, the compounds can be radiolabeled with radioactive isotopes, such as for example tritium (³H), iodine-125 (¹²⁵I) or carbon-14 (¹⁴C). In other embodiments, one or more carbon atoms is replaced with a silicon atom. All isotopic variations of the compounds disclosed herein, whether radioactive or not, are contemplated herein.

In some embodiments, the peptidomimetic macrocycle comprises an amino acid sequence that is at least 60%, at least 65%, at least 70%, at least 75%, at least 80%, at least 85%, at least 90%, or at least 95% identical to an amino acid sequence listed in Table 1, Table 1a, Table 1b, Table 1c, Table 2a, or Table 2b. In some embodiments, the peptidomimetic macrocycle comprises an amino acid sequence that is at least 60% identical to an amino acid sequence listed in Table 1, Table 1a, Table 1b, Table 1c, Table 2a, or Table 2b. In some embodiments, the peptidomimetic macrocycle comprises an amino acid sequence that is at least 65% identical to an amino acid sequence listed in Table 1, Table 1a, Table 1b, Table 1c, Table 2a, or Table 2b. In some embodiments, the peptidomimetic macrocycle comprises an amino acid sequence that is at least 70% identical to an amino acid sequence listed in Table 1, Table 1a, Table 1b, Table 1c, Table 2a, or Table 2b. In some embodiments, the peptidomimetic macrocycle comprises an amino acid sequence that is at least 75% identical to an amino acid sequence listed in Table 1, Table 1a, Table 1b, Table 1c, Table 2a, or Table 2b.

In some embodiments, the peptidomimetic macrocycle is at least 60%, at least 65%, at least 70%, at least 75%, at least 80%, at least 85%, at least 90%, or at least 95% identical to an amino acid sequence listed in Table 1, Table 1a, Table 1b, Table 1c, Table 2a, or Table 2b. In some embodiments, the peptidomimetic macrocycle is at least 60% identical to an amino acid sequence listed in Table 1, Table 1a, Table 1b, Table 1c, Table 2a, or Table 2b. In some embodiments, the peptidomimetic macrocycle is at least 65% identical to an amino acid sequence listed in Table 1, Table 1a, Table 1b, Table 1c, Table 2a, or Table 2b. In some embodiments, the peptidomimetic macrocycle is at least 70% identical to an amino acid sequence listed in Table 1, Table 1a, Table 1b, Table 1c, Table 2a, or Table 2b. In some embodiments, the peptidomimetic macrocycle is at least 75% identical to an amino acid sequence listed in Table 1, Table 1a, Table 1b, Table 1c, Table 2a, or Table 2b.

### Preparation of Peptidomimetic Macrocycles

Peptidomimetic macrocycles can be prepared by any of a variety of methods known in the art. For example, any of the residues indicated by “$” or “$r8” in Table 1, Table 1a, Table 1b, Table 1c, Table 2a, or Table 2b can be substituted with a residue capable of forming a crosslinker with a second residue in the same molecule or a precursor of such a residue.

α,α-Disubstituted amino acids and amino acid precursors can be employed in synthesis of the peptidomimetic macrocycle precursor polypeptides. For example, the “S5-olefin amino acid” is (S)-α-(2′-pentenyl) alanine and the “R8 olefin amino acid” is (R)-α-(2′-octenyl) alanine. Following incorporation of such amino acids into precursor polypeptides, the terminal olefins are reacted with a metathesis catalyst, leading to the formation of the peptidomimetic macrocycle. In various embodiments, the following amino acids can be employed in the synthesis of the peptidomimetic macrocycle:

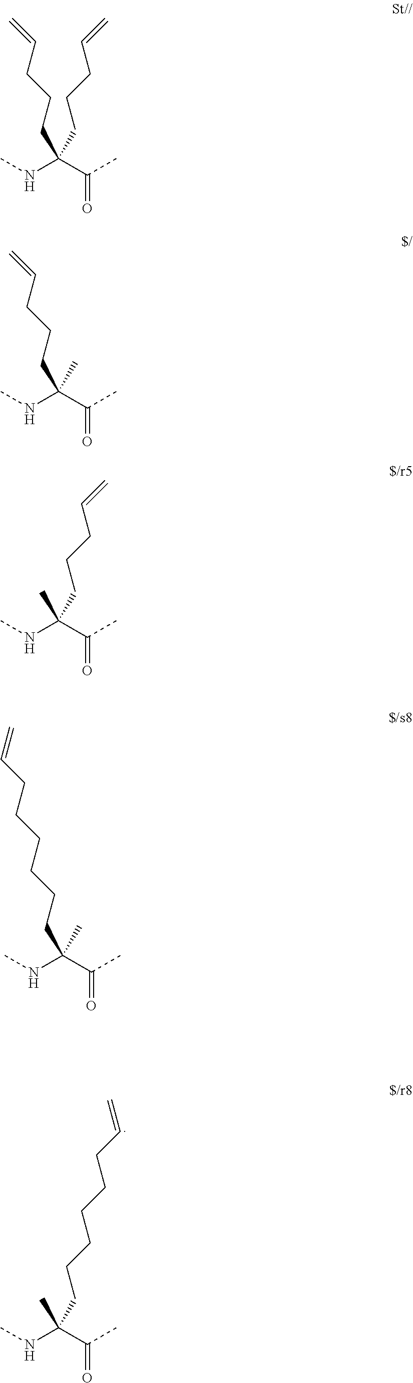

In other embodiments, the peptidomimetic macrocycles are of Formula IV or IVa. In such embodiments, amino acid precursors are used containing an additional substituent R—at the alpha position. Such amino acids are incorporated into the macrocycle precursor at the desired positions, which can be at the positions where the crosslinker is substituted or, alternatively, elsewhere in the sequence of the macrocycle precursor. Cyclization of the precursor is then effected according to the indicated method.

### Pharmaceutically-Acceptable Salts

The invention provides the use of pharmaceutically-acceptable salts of any therapeutic compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the invention. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the invention. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrrazole, pipyrrazole, imidazole, pyrazine, or pipyrazine.

In some embodiments, an ammonium salt is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrrazole salt, a pipyrrazole salt, an imidazole salt, a pyrazine salt, or a pipyrazine salt.

Acid addition salts can arise from the addition of an acid to a compound of the invention. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate and undecanoate. Salts derived from appropriate bases include alkali metal (e.g., sodium), alkaline earth metal (e.g., magnesium), ammonium and N-(alkyl)₄⁺ salts.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate (mesylate) salt, an ethanesulfonate salt, a benzenesulfonate salt, a p-toluenesulfonate salt, a citrate salt, an oxalate salt, or a maleate salt.

### Purity of Compounds of the Invention

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

### Formulation and Administration

### Pharmaceutical Compositions

Pharmaceutical compositions disclosed herein include peptidomimetic macrocycles and pharmaceutically-acceptable derivatives or prodrugs thereof. A “pharmaceutically-acceptable derivative” means any pharmaceutically-acceptable salt, ester, salt of an ester, pro-drug or other derivative of a compound disclosed herein which, upon administration to a recipient, is capable of providing (directly or indirectly) a compound disclosed herein. Particularly favored pharmaceutically-acceptable derivatives are those that increase the bioavailability of the compounds when administered to a mammal (e.g., by increasing absorption into the blood of an orally administered compound) or which increases delivery of the active compound to a biological compartment (e.g., the brain or lymphatic system) relative to the parent species. Some pharmaceutically-acceptable derivatives include a chemical group which increases aqueous solubility or active transport across the gastrointestinal mucosa.

In some embodiments, peptidomimetic macrocycles are modified by covalently or non-covalently joining appropriate functional groups to enhance selective biological properties. Such modifications include those which increase biological penetration into a given biological compartment (e.g., blood, lymphatic system, central nervous system), increase oral availability, increase solubility to allow administration by injection, alter metabolism, and alter rate of excretion.

For preparing pharmaceutical compositions from the compounds disclosed herein, pharmaceutically-acceptable carriers include either solid or liquid carriers. Solid form preparations include powders, tablets, pills, capsules, cachets, suppositories, and dispersible granules. A solid carrier can be one or more substances, which also acts as diluents, flavoring agents, binders, preservatives, tablet disintegrating agents, or an encapsulating material.

In powders, the carrier is a finely divided solid, which is in a mixture with the finely divided active component. In tablets, the active component is mixed with the carrier having the necessary binding properties in suitable proportions and compacted in the shape and size desired.

Suitable solid excipients are carbohydrate or protein fillers include, but are not limited to sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents are added, such as the crosslinked polyvinyl pyrrolidone, agar, alginic acid, or a salt thereof, such as sodium alginate.

Liquid form preparations include solutions, suspensions, and emulsions, for example, water or water/propylene glycol solutions. For parenteral injection, liquid preparations can be formulated in solution in aqueous polyethylene glycol solution.

The pharmaceutical preparation can be in unit dosage form. In such form the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

When one or more compositions disclosed herein comprise a combination of a peptidomimetic macrocycle and one or more additional therapeutic or prophylactic agents, both the compound and the additional agent should be present at dosage levels of between about 1 to 100%, and more preferably between about 5 to 95% of the dosage normally administered in a monotherapy regimen. In some embodiments, the additional agents are administered separately, as part of a multiple dose regimen, from one or more compounds disclosed herein. Alternatively, those agents are part of a single dosage form, mixed together with the compounds disclosed herein in a single composition.

### Mode of Administration

An effective amount of a peptidomimetic macrocycles of the disclosure can be administered in either single or multiple doses by any of the accepted modes of administration. In some embodiments, the peptidomimetic macrocycles of the disclosure are administered parenterally, for example, by subcutaneous, intramuscular, intrathecal, intravenous or epidural injection. For example, the peptidomimetic macrocycle is administered intravenously, intra-arterially, subcutaneously or by infusion. In some examples, the peptidomimetic macrocycle is administered intravenously. In some examples, the peptidomimetic macrocycle is administered intra-arterially.

Regardless of the route of administration selected, the peptidomimetic macrocycles of the present disclosure, and/or the pharmaceutical compositions of the present disclosure, are formulated into pharmaceutically-acceptable dosage forms. The peptidomimetic macrocycles according to the disclosure can be formulated for administration in any convenient way for use in human or veterinary medicine, by analogy with other pharmaceuticals.

In one aspect, the disclosure provides pharmaceutical formulation comprising a therapeutically-effective amount of one or more of the peptidomimetic macrocycles described above, formulated together with one or more pharmaceutically-acceptable carriers (additives) and/or diluents. In one embodiment, one or more of the peptidomimetic macrocycles described herein are formulated for parenteral administration for parenteral administration, one or more peptidomimetic macrocycles disclosed herein can be formulated as aqueous or non-aqueous solutions, dispersions, suspensions or emulsions or sterile powders which can be reconstituted into sterile injectable solutions or dispersions just prior to use. Such formulations can comprise sugars, alcohols, antioxidants, buffers, bacteriostats, solutes which render the formulation isotonic with the blood of the intended recipient or suspending or thickening agents. These compositions can also contain adjuvants such as preservatives, wetting agents, emulsifying agents and dispersing agents. Prevention of the action of microorganisms upon the subject compounds can be ensured by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin. If desired the formulation can be diluted prior to use with, for example, an isotonic saline solution or a dextrose solution. In some examples, the peptidomimetic macrocycle is formulated as an aqueous solution and is administered intravenously.

### Amount and Frequency of Administration

Dosing can be determined using various techniques. The selected dosage level can depend upon a variety of factors including the activity of the particular peptidomimetic macrocycle employed, the route of administration, the time of administration, the rate of excretion or metabolism of the particular peptidomimetic macrocycle being employed, the duration of the treatment, other drugs, compounds and/or materials used in combination with the particular peptidomimetic macrocycle employed, the age, sex, weight, condition, general health and prior medical history of the patient being treated, and like factors well known in the medical arts. The dosage values can also vary with the severity of the condition to be alleviated. For any particular subject, specific dosage regimens can be adjusted over time according to the individual need and the professional judgment of the person administering or supervising the administration of the compositions.

A physician or veterinarian can prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In some embodiments, a suitable daily dose of a peptidomimetic macrocycle of the disclosure can be that amount of the peptidomimetic macrocycle which is the lowest dose effective to produce a therapeutic effect. Such an effective dose will generally depend upon the factors described above. The precise time of administration and amount of any particular peptidomimetic macrocycle that will yield the most effective treatment in a given patient will depend upon the activity, pharmacokinetics, and bioavailability of a particular peptidomimetic macrocycle, physiological condition of the patient (including age, sex, disease type and stage, general physical condition, responsiveness to a given dosage and type of medication), route of administration, and the like.

Dosage can be based on the amount of the peptidomimetic macrocycle per kg body weight of the patient. Alternatively, the dosage of the subject disclosure can be determined by reference to the plasma concentrations of the peptidomimetic macrocycle. For example, the maximum plasma concentration (Cₘₐₓ) and the area under the plasma concentration-time curve from time 0 to infinity (AUC) can be used.

The amount of the peptidomimetic macrocycle that is administered to a subject can be from about 1 μg/kg, 25 μg/kg, 50 μg/kg, 75 μg/kg, 100 i g/kg, 125 μg/kg, 150 μg/kg, 175 μg/kg, 200 μg/kg, 225 μg/kg, 250 μg/kg, 275 μg/kg, 300 μg/kg, 325 μg/kg, 350 μg/kg, 375 μg/kg, 400 μg/kg, 425 μg/kg, 450 μg/kg, 475 μg/kg, 500 μg/kg, 525 μg/kg, 550 μg/kg, 575 μg/kg, 600 μg/kg, 625 μg/kg, 650 μg/kg, 675 μg/kg, 700 μg/kg, 725 μg/kg, 750 μg/kg, 775 μg/kg, 800 μg/kg, 825 μg/kg, 850 μg/kg, 875 μg/kg, 900 μg/kg, 925 μg/kg, 950 μg/kg, 975 μg/kg, 1 mg/kg, 2.5 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, or 100 mg/kg per body weight of the subject.

The amount of the peptidomimetic macrocycle that is administered to a subject can be from about 0.01 mg/kg to about 100 mg/kg body weight of the subject. In some embodiments, the amount of the peptidomimetic macrocycle administered is about 0.01-10 mg/kg, about 0.01-20 mg/kg, about 0.01-50 mg/kg, about 0.1-10 mg/kg, about 0.1-20 mg/kg, about 0.1-50 mg/kg, about 0.1-100 mg/kg, about 0.5-10 mg/kg, about 0.5-20 mg/kg, about 0.5-50 mg/kg, about 0.5-100 mg/kg, about 1-10 mg/kg, about 1-20 mg/kg, about 1-50 mg/kg, or about 1-100 mg/kg body weight of the human subject. In some embodiments, the amount of the peptidomimetic macrocycle administered is about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, or 20 mg/kg body weight of the subject. In some embodiments, the amount of the peptidomimetic macrocycle administered is about 5 mg/kg. In some embodiments, the amount of the peptidomimetic macrocycle administered is about 10 mg/kg. In some embodiments, the amount of the peptidomimetic macrocycle administered is about 15 mg/kg.

In some embodiments, the amount of the peptidomimetic macrocycle administered is about 0.16 mg, about 0.32 mg, about 0.64 mg, about 1.28 mg, about 3.56 mg, about 7.12 mg, about 14.24 mg, or about 20 mg per kilogram body weight of the subject. In some examples the amount of the peptidomimetic macrocycle administered is about 0.16 mg per kilogram body weight of the subject. In some examples the amount of the peptidomimetic macrocycle administered is about 0.32 mg per kilogram body weight of the subject. In some examples the amount of the peptidomimetic macrocycle administered is about 0.64 mg per kilogram body weight of the subject. In some examples the amount of the peptidomimetic macrocycle administered is about 1.28 mg per kilogram body weight of the subject. In some examples the amount of the peptidomimetic macrocycle administered is about 3.56 mg per kilogram body weight of the subject. In some examples the amount of the peptidomimetic macrocycle administered is about 7.12 mg per kilogram body weight of the subject. In some examples the amount of the peptidomimetic macrocycle administered is about 14.24 mg per kilogram body weight of the subject.

In some embodiments, a pharmaceutically-acceptable amount of a peptidomimetic macrocycle is administered to a subject 1, 2, 3, 4, 5, 6, 7, 8, 9, 10, 11, 12, 13, or 14 times a week. In some embodiments about 0.5-about 20 mg or about 0.5-about 10 mg of the peptidomimetic macrocycle per kilogram body weight of the human subject is administered once a week. For example about 0.5-about 1 mg, about 0.5-about 5 mg, about 0.5-about 10 mg, about 0.5-about 15 mg, about 1-about 5 mg, about 1-about 10 mg, about 1-about 15 mg, about 1-about 20 mg, about 5-about 10 mg, about 1-about 15 mg, about 5-about 20 mg, about 10-about 15 mg, about 10-about 20 mg, or about 15-about 20 mg of the peptidomimetic macrocycle per kilogram body weight of the human subject is administered once a week. In some examples about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, about 10 mg, about 10.25 mg, about 10.5 mg, about 10.75 mg, about 11 mg, about 11.25 mg, about 11.5 mg, about 11.75 mg, about 12 mg, about 12.25 mg, about 12.5 mg, about 12.75 mg, about 13 mg, about 13.25 mg, about 13.5 mg, about 13.75 mg, about 14 mg, about 14.25 mg, about 14.5 mg, about 14.75 mg, about 15 mg, about 15.25 mg, about 15.5 mg, about 15.75 mg, about 16 mg, about 16.5 mg, about 17 mg, about 17.5 mg, about 18 mg, about 18.5 mg, about 19 mg, about 19.5 mg, or about 20 mg of the peptidomimetic macrocycle per kilogram body weight of the human subject is administered once a week. In some examples, the amount of the peptidomimetic macrocycle administered is about 1.25 mg, about 2.5 mg, about 5 mg, about 10 mg, or about 20 mg per kilogram body weight of the human subject and the peptidomimetic macrocycle is administered once a week. In some examples, the amount of the peptidomimetic macrocycle administered is about 1.25 mg, about 2.5 mg, about 5 mg or about 10 mg per kilogram body weight of the human subject and the peptidomimetic macrocycle is administered once a week.

In some embodiments about 0.5-about 20 mg or about 0.5-about 10 mg of the peptidomimetic macrocycle per kilogram body weight of the human subject is administered two times a week. For example about 0.5-about 1 mg, about 0.5-about 5 mg, about 0.5-about 10 mg, about 0.5-about 15 mg, about 1-about 5 mg, about 1-about 10 mg, about 1-about 15 mg, about 1-about 20 mg, about 5-about 10 mg, about 1-about 15 mg, about 5-about 20 mg, about 10-about 15 mg, about 10-about 20 mg, or about 15-about 20 mg of the peptidomimetic macrocycle per kilogram body weight of the human subject is administered about twice a week. In some examples about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, about 10 mg, about 10.25 mg, about 10.5 mg, about 10.75 mg, about 11 mg, about 11.25 mg, about 11.5 mg, about 11.75 mg, about 12 mg, about 12.25 mg, about 12.5 mg, about 12.75 mg, about 13 mg, about 13.25 mg, about 13.5 mg, about 13.75 mg, about 14 mg, about 14.25 mg, about 14.5 mg, about 14.75 mg, about 15 mg, about 15.25 mg, about 15.5 mg, about 15.75 mg, about 16 mg, about 16.5 mg, about 17 mg, about 17.5 mg, about 18 mg, about 18.5 mg, about 19 mg, about 19.5 mg, or about 20 mg of the peptidomimetic macrocycle per kilogram body weight of the human subject is administered two times a week. In some examples, the amount of the peptidomimetic macrocycle administered is about 1.25 mg, about 2.5 mg, about 5 mg, about 10 mg, or about 20 mg per kilogram body weight of the human subject and the peptidomimetic macrocycle is administered two times a week. In some examples, the amount of the peptidomimetic macrocycle administered is about 1.25 mg, about 2.5 mg, about 5 mg or about 10 mg per kilogram body weight of the human subject and the peptidomimetic macrocycle is administered two times a week.

In some embodiments about 0.5-about 20 mg or about 0.5-about 10 mg of the peptidomimetic macrocycle per kilogram body weight of the human subject is administered 3, 4, 5, 6, or 7 times a week. For example, about 0.5-about 1 mg, about 0.5-about 5 mg, about 0.5-about 10 mg, about 0.5-about 15 mg, about 1-about 5 mg, about 1-about 10 mg, about 1-about 15 mg, about 1-about 20 mg, about 5-about 10 mg, about 1-about 15 mg, about 5-about 20 mg, about 10-about 15 mg, about 10-about 20 mg, or about 15-about 20 mg of the peptidomimetic macrocycle per kilogram body weight of the human subject is administered 3, 4, 5, 6, or 7 times a week. In some examples about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, about 10 mg, about 10.25 mg, about 10.5 mg, about 10.75 mg, about 11 mg, about 11.25 mg, about 11.5 mg, about 11.75 mg, about 12 mg, about 12.25 mg, about 12.5 mg, about 12.75 mg, about 13 mg, about 13.25 mg, about 13.5 mg, about 13.75 mg, about 14 mg, about 14.25 mg, about 14.5 mg, about 14.75 mg, about 15 mg, about 15.25 mg, about 15.5 mg, about 15.75 mg, about 16 mg, about 16.5 mg, about 17 mg, about 17.5 mg, about 18 mg, about 18.5 mg, about 19 mg, about 19.5 mg, or about 20 mg of the peptidomimetic macrocycle per kilogram body weight of the human subject is administered 3, 4, 5, 6, or 7 times a week. In some examples, the amount of the peptidomimetic macrocycle administered is about 1.25 mg, about 2.5 mg, about 5 mg, about 10 mg, or about 20 mg per kilogram body weight of the human subject and the peptidomimetic macrocycle is administered 3, 4, 5, 6, or 7 times a week. In some examples, the amount of the peptidomimetic macrocycle administered is about 1.25 mg, about 2.5 mg, about 5 mg, or about 10 mg per kilogram body weight of the human subject and the peptidomimetic macrocycle is administered 3, 4, 5, 6, or 7 times a week.

In some embodiments, a pharmaceutically-acceptable amount of a peptidomimetic macrocycle is administered to a subject once every 1, 2, 3, 4, 5, 6, 7, or 8 weeks. In some embodiments, about 0.5-about 20 mg or about 0.5-about 10 mg of the peptidomimetic macrocycle per kilogram body weight of the human subject is administered once every 2, 3, or 4 weeks. For example, about 0.5-about 1 mg, about 0.5-about 5 mg, about 0.5-about 10 mg, about 0.5-about 15 mg, about 1-about 5 mg, about 1-about 10 mg, about 1-about 15 mg, about 1-about 20 mg, about 5-about 10 mg, about 1-about 15 mg, about 5-about 20 mg, about 10-about 15 mg, about 10-about 20 mg, or about 15-about 20 mg of the peptidomimetic macrocycle per kilogram body weight of the human subject is administrated 3, 4, 5, 6, or 7 once every 2 or 3 week. In some examples about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, about 10 mg, about 10.25 mg, about 10.5 mg, about 10.75 mg, about 11 mg, about 11.25 mg, about 11.5 mg, about 11.75 mg, about 12 mg, about 12.25 mg, about 12.5 mg, about 12.75 mg, about 13 mg, about 13.25 mg, about 13.5 mg, about 13.75 mg, about 14 mg, about 14.25 mg, about 14.5 mg, about 14.75 mg, about 15 mg, about 15.25 mg, about 15.5 mg, about 15.75 mg, about 16 mg, about 16.5 mg, about 17 mg, about 17.5 mg, about 18 mg, about 18.5 mg, about 19 mg, about 19.5 mg, or about 20 mg of the peptidomimetic macrocycle per kilogram body weight of the human subject is administered once every 2 or 3 weeks. In some examples, the amount of the peptidomimetic macrocycle administered is about 1.25 mg, about 2.5 mg, about 5 mg, about 10 mg, or about 20 mg per kilogram body weight of the human subject and the peptidomimetic macrocycle is administered once every 2 weeks. In some examples, the amount of the peptidomimetic macrocycle administered is about 1.25 mg, about 2.5 mg, about 5 mg or about 10 mg per kilogram body weight of the human subject and the peptidomimetic macrocycle is administered once every 2 weeks. In some examples, the amount of the peptidomimetic macrocycle administered is about 1.25 mg, about 2.5 mg, about 5 mg, about 10 mg, or about 20 mg per kilogram body weight of the human subject and the peptidomimetic macrocycle is administered once every 3 weeks. In some examples, the amount of the peptidomimetic macrocycle administered is about 1.25 mg, about 2.5 mg, about 5 mg, or about 10 mg per kilogram body weight of the human subject and the peptidomimetic macrocycle is administered once every 3 weeks.

In some embodiments, a pharmaceutically-acceptable amount of a peptidomimetic macrocycle is administered to a subject gradually over a period of time. In some embodiments, an amount of a peptidomimetic macrocycle can be administered to a subject gradually over a period of from about 0.1 h to about 24 h. In some embodiments, an amount of a peptidomimetic macrocycle can be administered to a subject over a period of about 0.1 h, about 0.2 h, about 0.3 h, about 0.4 h, about 0.5 h, about 0.6 h, about 0.7 h, about 0.8 h, about 0.9 h, about 1 h, about 1.5 h, about 2 h, about 2.5 h, about 3 h, about 3.5 h, about 4 h, about 4.5 h, about 5 h, about 5.5 h, about 6 h, about 6.5 h, about 7 h, about 7.5 h, about 8 h, about 8.5 h, about 9 h, about 9.5 h, about 10 h, about 10.5 h, about 11 h, about 11.5 h, about 12 h, about 12.5 h, about 13 h, about 13.5 h, about 14 h, about 14.5 h, about 15 h, about 15.5 h, about 16 h, about 16.5 h, about 17 h, about 17.5 h, about 18 h, about 18.5 h, about 19 h, about 19.5 h, about 20 h, about 20.5 h, about 21 h, about 21.5 h, about 22 h, about 22.5 h, about 23 h, about 23.5 h, or about 24 h. In some embodiments, a pharmaceutically-acceptable amount of a peptidomimetic macrocycle is administered gradually over a period of about 0.5 h. In some embodiments, a pharmaceutically-acceptable amount of a peptidomimetic macrocycle is administered gradually over a period of about 1 h. In some embodiments, a pharmaceutically-acceptable amount of a peptidomimetic macrocycle is administered gradually over a period of about 1.5 h.

Administration of the peptidomimetic macrocycles can continue for as long as clinically necessary. In some embodiments, a peptidomimetic macrocycle of the disclosure can be administered for more than 1 day, more than 1 week, more than 1 month, more than 2 months, more than 3 months, more than 4 months, more than 5 months, more than 6 months, more than 7 months, more than 8 months, more than 9 months, more than 10 months, more than 11 months, more than 12 months, more than 13 months, more than 14 months, more than 15 months, more than 16 months, more than 17 months, more than 18 months, more than 19 months, more than 20 months, more than 21 months, more than 22 months, more than 23 months, or more than 24 months. In some embodiments, one or more peptidomimetic macrocycle of the disclosure is administered for less than 1 week, less than 1 month, less than 2 months, less than 3 months, less than 4 months, less than 5 months, less than 6 months, less than 7 months, less than 8 months, less than 9 months, less than 10 months, less than 11 months, less than 12 months, less than 13 months, less than 14 months, less than 15 months, less than 16 months, less than 17 months, less than 18 months, less than 19 months, less than 20 months, less than 21 months, less than 22 months, less than 23 months, or less than 24 months.

In some embodiments, a peptidomimetic macrocycle can be administered to a subject 1, 2, 3, 4, 5, 6, 7, 8, 9, 10, 11, 12, 13, 14, 15, 16, 17, 18, 19, or 20 times over a treatment cycle. In some embodiments a peptidomimetic macrocycle can be administered to a subject 2, 4, 6, or 8 times over a treatment cycle. In some embodiments, a peptidomimetic macrocycle can be administered to a subject 4 times over a treatment cycle. In some embodiments, a treatment cycle is 7 days, 14 days, 21 days, or 28 days long. In some embodiments, a treatment cycle is 21 days long. In some embodiments, a treatment cycle is 28 days long.

In some embodiments, a peptidomimetic macrocycle is administered on day 1, 8, 15 and 28 of a 28 day cycle. In some embodiments, the peptidomimetic macrocycle is administered on day 1, 8, 15 and 28 of a 28 day cycle and administration is continued for two cycles. In some embodiments, the peptidomimetic macrocycle is administered on day 1, 8, 15 and 28 of a 28 day cycle and administration is continued for three cycles. In some embodiments, the peptidomimetic macrocycle is administered on day 1, 8, 15 and 28 of a 28 day cycle and administration is continued for 4, 5, 6, 7, 8, 9, 10, or more than 10 cycles.

In some embodiments, the peptidomimetic macrocycle is administered on day 1, 8, 11 and 21 of a 21-day cycle. In some embodiments, the peptidomimetic macrocycle is administered on day 1, 8, 11 and 21 of a 21-day cycle and administration is continued for two cycles. In some embodiments, the peptidomimetic macrocycle is administered on day 1, 8, 11 and 21 of a 21-day cycle and administration is continued for three cycles. In some embodiments, the peptidomimetic macrocycle is administered on day 1, 8, 11 and 21 of a 21-day cycle and administration is continued for 4, 5, 6, 7, 8, 9, 10, or more than 10 cycles.

In some embodiments, one or more peptidomimetic macrocycle of the disclosure is administered chronically on an ongoing basis. In some embodiments administration of one or more peptidomimetic macrocycle of the disclosure is continued until documentation of disease progression, unacceptable toxicity, or patient or physician decision to discontinue administration.

In some embodiments, the compounds of the invention can be used to treat one condition. In some embodiments, the compounds of the invention can be used to treat two conditions. In some embodiments, the compounds of the invention can be used to treat three conditions. In some embodiments, the compounds of the invention can be used to treat four conditions. In some embodiments, the compounds of the invention can be used to treat five conditions.

### Methods of Use

In one aspect, provided herein are novel peptidomimetic macrocycles that are useful in competitive binding assays to identify agents which bind to the natural ligand(s) of the proteins or peptides upon which the peptidomimetic macrocycles are modeled. For example, in the p53/MDMX system, labeled peptidomimetic macrocycles based on p53 can be used in a MDMX binding assay along with small molecules that competitively bind to MDMX. Competitive binding studies allow for rapid in vitro evaluation and determination of drug candidates specific for the p53/MDMX system. Such binding studies can be performed with any of the peptidomimetic macrocycles disclosed herein and their binding partners. Further provided are methods for the generation of antibodies against the peptidomimetic macrocycles. In some embodiments, these antibodies specifically bind both the peptidomimetic macrocycle and the precursor peptides, such as p53, to which the peptidomimetic macrocycles are related. Such antibodies, for example, disrupt the native protein-protein interaction, for example, binding between p53 and MDMX.

In other aspects, provided herein are both prophylactic and therapeutic methods of treating a subject at risk of (or susceptible to) a disorder or having a disorder associated with aberrant (e.g., insufficient or excessive) expression or activity of the molecules including p53, MDM2 or MDMX.

In another embodiment, a disorder is caused, at least in part, by an abnormal level of p53 or MDM2 or MDMX, (e.g., over or under expression), or by the presence of p53 or MDM2 or MDMX exhibiting abnormal activity. As such, the reduction in the level and/or activity of p53 or MDM2 or MDMX, or the enhancement of the level and/or activity of p53 or MDM2 or MDMX, by peptidomimetic macrocycles derived from p53, is used, for example, to ameliorate or reduce the adverse symptoms of the disorder.

In another aspect, provided herein are methods for treating or preventing a disease including hyperproliferative disease and inflammatory disorder by interfering with the interaction or binding between binding partners, for example, between p53 and MDM2 or p53 and MDMX. These methods comprise administering an effective amount of a compound to a warm blooded animal, including a human. In some embodiments, the administration of one or more compounds disclosed herein induces cell growth arrest or apoptosis.

In some embodiments, the peptidomimetic macrocycles can be used to treat, prevent, and/or diagnose cancers and neoplastic conditions. As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states can be categorized as pathologic, i.e., characterizing or constituting a disease state, or can be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. A metastatic tumor can arise from a multitude of primary tumor types, including but not limited to those of breast, lung, liver, colon and ovarian origin. “Pathologic hyperproliferative” cells occur in disease states characterized by malignant tumor growth. Examples of non-pathologic hyperproliferative cells include proliferation of cells associated with wound repair. Examples of cellular proliferative and/or differentiation disorders include cancer, e.g., carcinoma, sarcoma, or metastatic disorders. In some embodiments, the peptidomimetic macrocycles are novel therapeutic agents for controlling breast cancer, ovarian cancer, colon cancer, lung cancer, metastasis of such cancers and the like.

Examples of cancers or neoplastic conditions include, but are not limited to, a fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastric cancer, esophageal cancer, rectal cancer, pancreatic cancer, ovarian cancer, prostate cancer, uterine cancer, cancer of the head and neck, skin cancer, brain cancer, squamous cell carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular cancer, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, or Kaposi sarcoma.

In some embodiments, the cancer is head and neck cancer, melanoma, lung cancer, breast cancer, or glioma.

Examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. The diseases can arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), periphieral T-cell lymphoma (PTCL), large granular lymphocytic leukemia (LGF), Hodgkin's disease and Reed-Stemberg disease.

Examples of cellular proliferative and/or differentiation disorders of the breast include, but are not limited to, proliferative breast disease including, e.g., epithelial hyperplasia, sclerosing adenosis, and small duct papillomas; tumors, e.g., stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

Examples of cellular proliferative and/or differentiative disorders of the skin include, but are not limited to proliferative skin disease such as melanomas, including mucosal melanoma, superficial spreading melanoma, nodular melanoma, lentigo (e.g. lentigo maligna, lentigo maligna melanoma, or acral lentiginous melanoma), amelanotic melanoma, desmoplastic melanoma, melanoma with features of a Spitz nevus, melanoma with small nevus-like cells, polypoid melanoma, and soft-tissue melanoma; basal cell carcinomas including micronodular basal cell carcinoma, superficial basal cell carcinoma, nodular basal cell carcinoma (rodent ulcer), cystic basal cell carcinoma, cicatricial basal cell carcinoma, pigmented basal cell carcinoma, aberrant basal cell carcinoma, infiltrative basal cell carcinoma, nevoid basal cell carcinoma syndrome, polypoid basal cell carcinoma, pore-like basal cell carcinoma, and fibroepithelioma of Pinkus; squamus cell carcinomas including acanthoma (large cell acanthoma), adenoid squamous cell carcinoma, basaloid squamous cell carcinoma, clear cell squamous cell carcinoma, signet-ring cell squamous cell carcinoma, spindle cell squamous cell carcinoma, Marjolin's ulcer, erythroplasia of Queyrat, and Bowen's disease; or other skin or subcutaneous tumors.

Examples of cellular proliferative and/or differentiation disorders of the lung include, but are not limited to, bronchogenic carcinoma, including paraneoplastic syndromes, bronchioloalveolar carcinoma, neuroendocrine tumors, such as bronchial carcinoid, miscellaneous tumors, and metastatic tumors; pathologies of the pleura, including inflammatory pleural effusions, noninflammatory pleural effusions, pneumothorax, and pleural tumors, including solitary fibrous tumors (pleural fibroma) and malignant mesothelioma.

Examples of cellular proliferative and/or differentiative disorders of the colon include, but are not limited to, non-neoplastic polyps, adenomas, familial syndromes, colorectal carcinogenesis, colorectal carcinoma, and carcinoid tumors.

Examples of cellular proliferative and/or differentiative disorders of the liver include, but are not limited to, nodular hyperplasias, adenomas, and malignant tumors, including primary carcinoma of the liver and metastatic tumors.

Examples of cellular proliferative and/or differentiative disorders of the ovary include, but are not limited to, ovarian tumors such as, tumors of coelomic epithelium, serous tumors, mucinous tumors, endometrioid tumors, clear cell adenocarcinoma, cystadenofibroma, Brenner tumor, surface epithelial tumors; germ cell tumors such as mature (benign) teratomas, monodermal teratomas, immature malignant teratomas, dysgerminoma, endodermal sinus tumor, choriocarcinoma; sex cord-stomal tumors such as, granulosa-theca cell tumors, thecomafibromas, androblastomas, hill cell tumors, and gonadoblastoma; and metastatic tumors such as Krukenberg tumors.

### Combination Treatment

Combination therapy with a peptidomimetic macrocycle of the disclosure and at least one additional therapeutic agent, for example, any additional therapeutic agent described herein, can be used to treat a condition. In some embodiments, the combination therapy can produce a significantly better therapeutic result than the additive effects achieved by each individual constituent when administered alone at a therapeutic dose. In some embodiments, the dosage of the peptidomimetic macrocycle or additional therapeutic agent, for example, any additional therapeutic agent described herein, in combination therapy can be reduced as compared to monotherapy with each agent, while still achieving an overall therapeutic effect. In some embodiments, a peptidomimetic macrocycle and an additional therapeutic agent, for example, any additional therapeutic agent described herein, can exhibit a synergistic effect. In some embodiments, the synergistic effect of a peptidomimetic macrocycle and additional therapeutic agent, for example, any additional therapeutic agent described herein, can be used to reduce the total amount drugs administered to a subject, which decrease side effects experienced by the subject.

The peptidomimetic macrocycles of the disclosure can be used in combination with at least one additional pharmaceutically-active agent, for example, any additional therapeutic agent described herein. In some embodiments, the at least one additional pharmaceutically-active agent, for example, any additional therapeutic agent described herein, can modulate the same or a different target as the peptidomimetic macrocycles of the disclosure. In some embodiments, the at least one additional pharmaceutically-active agent, for example, any additional therapeutic agent described herein, can modulate the same target as the peptidomimetic macrocycles of the disclosure, or other components of the same pathway, or overlapping sets of target enzymes. In some embodiments, the at least one additional pharmaceutically-active agent, for example, any additional therapeutic agent described herein, can modulate a different target from the peptidomimetic macrocycles of the disclosure.

Accordingly, in one aspect, the present disclosure provides a method for treating cancer, the method comprising administering to a subject in need thereof (a) an effective amount of a peptidomimetic macrocycle of the disclosure and (b) an effective amount of at least one additional pharmaceutically active agent, for example, any additional therapeutic agent described herein, to provide a combination therapy. In some embodiments, the combination therapy may have an enhanced therapeutic effect compared to the effect of the peptidomimetic macrocycle and the at least one additional pharmaceutically active agent each administered alone. According to certain exemplary embodiments, the combination therapy has a synergistic therapeutic effect. According to this embodiment, the combination therapy produces a significantly better therapeutic result (e.g., anti-cancer, cell growth arrest, apoptosis, induction of differentiation, cell death, etc.) than the additive effects achieved by each individual constituent when administered alone at a therapeutic dose.

Combination therapy includes but is not limited to the combination of peptidomimetic macrocycles of this disclosure with chemotherapeutic agents, therapeutic antibodies, and radiation treatment, to provide a synergistic therapeutic effect. In some embodiments, the peptidomimetic macrocycles of the disclosure are used in combination with one or more anti-cancer (antineoplastic or cytotoxic) chemotherapy drug. Suitable chemotherapeutic agents for use in the combinations of the present disclosure include, but are not limited to, alkylating agents, antibiotic agents, antimetabolic agents, hormonal agents, plant-derived agents, anti-angiogenic agents, differentiation inducing agents, cell growth arrest inducing agents, apoptosis inducing agents, cytotoxic agents, agents affecting cell bioenergetics, biologic agents, e.g., monoclonal antibodies, kinase inhibitors and inhibitors of growth factors and their receptors, gene therapy agents, cell therapy, or any combination thereof.

In some embodiments, a method of treating cancer in a subject in need thereof can comprise administering to the subject a therapeutically effective amount of a p53 agent that inhibits the interaction between p53 and MDM2 and/or p53 and MDMX, and/or modulates the activity of p53 and/or MDM2 and/or MDMX; and at least one additional pharmaceutically-active agent. In some examples, the p53 agent is selected from the group consisting of a small organic or inorganic molecule; a saccharine; an oligosaccharide; a polysaccharide; a peptide, a protein, a peptide analog, a peptide derivative; an antibody, an antibody fragment, a peptidomimetic; a peptidomimetic macrocycle of the disclosure; a nucleic acid; a nucleic acid analog, a nucleic acid derivative; an extract made from biological materials; a naturally-occurring or synthetic composition; and any combination thereof.

In some embodiments, the p53 agent is selected from the group consisting of RG7388 (RO5503781, idasanutlin), RG7112 (RO5045337), nutlin3a, nutlin3b, nutlin3, nutlin2, spirooxindole containing small molecules, 1,4-diazepines, 1,4-benzodiazepine-2,5-dione compounds, WK23, WK298, SJ172550, RO2443, RO5963, RO5353, RO2468, MK8242 (SCH900242), M1888, M1773 (SAR405838), NVPCGM097, DS3032b, AM8553, AMG232, NSC207895 (X1006), JNJ26854165 (serdemetan), RITA (NSC652287), YH239EE, or any combination thereof. In some examples, the at least one additional pharmaceutically-active agent is selected from the group consisting of palbociclib (PD0332991); abemaciclib (LY2835219); ribociclib (LEE 011); voruciclib (P1446A-05); fascaplysin; arcyriaflavin; 2-bromo-12,13-dihydro-5H-indolo[2,3-a]pyrrolo[3,4-c]carbazole-5,7(6H)-dione; 3-amino thioacridone (3-ATA), trans-4-((6-(ethylamino)-2-((1-(phenylmethyl)-1H-indol-5-yl)amino)-4-pyrimidinyl)amino)-cyclohexano (CINK4); 1,4-dimethoxyacridine-9(10H)-thione (NSC 625987); 2-methyl-5-(p-tolylamino)benzo[d]thiazole-4,7-dione (ryuvidine); and flavopiridol (alvocidib); and any combination thereof.

a. Combination Treatment with Estrogen Receptor Antagonists

In some embodiments, the peptidomimetic macrocycles of the disclosure are used in combination with an estrogen receptor antagonist. In some embodiments, the peptidomimetic macrocycles of the disclosure are used in combination with toremifene (Fareston®), fulvestrant (Faslodex®), or tamoxifen citrate (Soltamox®).

Fulvestrant is a selective estrogen receptor degrader (SERD) and is indicated for the treatment of hormone receptor positive metastatic breast cancer in postmenopausal women with disease progression following anti-estrogen therapy. Fulvestrant is a complete estrogen receptor antagonist with little to no agonist effects and accelerates the proteasomal degradation of the estrogen receptor. Fulvestrant has poor oral bioavailability and is administered via intramuscular injection. Fulvestrant-induced expression of ErbB3 and ErbB4 receptors sensitizes oestrogen receptor-positive breast cancer cells to heregulin beta1. In some embodiments, the peptidomimetic macrocycles of the disclosure are used in combination with fulvestrant.

b. Combination Treatment with Aromatase Inhibitors

In some embodiments, the peptidomimetic macrocycles of the disclosure are used in combination with an aromatase inhibitor. Aromatase inhibitors are used in the treatment of breast cancer in post-menopausal women and gynecomastia in men. Aromatase inhibitors can be used off-label to reduce estrogen conversion when using external testosterone. Aromatase inhibitors can also be used for chemoprevention in high-risk women.

In some embodiments, the peptidomimetic macrocycles of the disclosure are used in combination with a non-selective aromatase inhibitor. In some embodiments, the peptidomimetic macrocycles of the disclosure can be used in combination with a non-selective aromatase inhibitor, such as aminoglutethimide or testolactone (Teslac®). In some embodiments, the peptidomimetic macrocycles of the disclosure are used in combination with a selective aromatase inhibitor. In some embodiments, the peptidomimetic macrocycles of the disclosure are used in combination with a selective aromatase inhibitor, such as anastrozole (Arimidex®), letrozole (Femara®), exemestane (Aromasin®), vorozole (Rivizor®), formestane (Lentaron®), or fadrozole (Afema®). In some embodiments, the peptidomimetic macrocycles of the disclosure are used in combination with exemestane. In some embodiments, the peptidomimetic macrocycles of the disclosure are used in combination with an aromatase inhibitor that has unknown mechanism of action, such as 1,4,6-androstatrien-3,17-dione (ATD) or 4-androstene-3,6,17-trione.

c. Combination Treatment with mTOR Inhibitors

In some embodiments, the peptidomimetic macrocycles of the disclosure are used in combination with an mTOR inhibitor. mTOR inhibitors are drugs that inhibit the mechanistic target of rapamycin (mTOR), which is a serine/threonine-specific protein kinase that belongs to the family of phosphatidylinositol-3 kinase (PI3K)-related kinases (PIKKs). mTOR regulates cellular metabolism, growth, and proliferation by forming and signaling through the protein complexes mTORC1 and mTORC2.

In some embodiments, the peptidomimetic macrocycles of the disclosure are used in combination with an mTOR inhibitor, such as rapamycin, temsirolimus (CCI-779), everolimus (RAD001), ridaforolimus (AP-23573). In some embodiments, the peptidomimetic macrocycles of the disclosure are used in combination with everolimus (Afinitor®). Everolimus affects the mTORC1 protein complex and can lead to hyper-activation of the kinase AKT, which can lead to longer survival in some cell types. Everolimus binds to FKBP12, a protein receptor which directly interacts with mTORC1 and inhibits downstream signaling. mRNAs that codify proteins implicated in the cell cycle and in the glycolysis process are impaired or altered as a result, inhibiting tumor growth and proliferation.

In some embodiments, the peptidomimetic macrocycles of the disclosure are used in combination with a mTOR inhibitor and an aromatase inhibitor. For example, the peptidomimetic macrocycles can be used in combination with everolimus and exemestane.

d. Combination Treatment with Antimetabolites

Antimetabolites are chemotherapy treatments that are similar to normal substances within the cell. When cells incorporate the antimetabolites into the cellular metabolism, the cells are unable to divide. Antimetabolites are cell-cycle specific and attack cells at specific phases in the cell cycle.

In some examples, the peptidomimetic macrocycles of the disclosure are used in combination with one or more antimetabolites, such as a folic acid antagonist, pyrimidine antagonist, purine antagonist, or an adenosine deaminase inhibitor. In some embodiments, the peptidomimetic macrocycles of the disclosure are used in combination with an antimetabolite, such as methotrexate, 5-fluorouracil, foxuridine, cytarabine, capecitabine, gemcitabine, 6-mercaptopurine, 6-thioguanine, cladribine, fludarabine, nelarabine, or pentostatin. In some embodiments, the peptidomimetic macrocycles of the disclosure are used in combination with capecitabine (Xeloda®), gemcitabine (Gemzar®), or cytarabine (Cytosar-U®).

e. Combination Treatment with Plant Alkaloids

In some embodiments, the peptidomimetic macrocycles of the disclosure are used in combination with plant alkaloids. In some embodiments, the peptidomimetic macrocycles of the disclosure are used in combination with plant alkaloids, such as vinca alkaloids, taxanes, podophyllotoxins, or camptothecan analogues. In some embodiments, the peptidomimetic macrocycles of the disclosure are used in combination with plant alkaloids, such as vincristine, vinblastine, vinorelbine, paclitaxel, docetaxel, etoposide, tenisopide, irinotecan, or topotecan.

In some embodiments, the peptidomimetic macrocycles of the disclosure are used in combination with taxanes, such as paclitaxel (Abraxane® or Taxol®) and docetaxel (Taxotere®). In some embodiments, the peptidomimetic macrocycles of the instant disclosure are used in combination with paclitaxel. In some embodiments, the peptidomimetic macrocycles of the instant disclosure are used in combination with docetaxel.

f. Combination Treatment with Therapeutic Antibodies

In some embodiments, the peptidomimetic macrocycles of the disclosure are used in combination with therapeutic antibodies. In some embodiments, the peptidomimetic macrocycles of the disclosure are used in combination with naked monoclonal antibodies, such as alemtuzumab (Campath®) or trastuzumab (Herceptin®). In some embodiments, the peptidomimetic macrocycles of the disclosure are used in combination with conjugated monoclonal antibodies, such as radiolabeled antibodies or chemolabeled antibodies. In some embodiments, the peptidomimetic macrocycles of the disclosure are used in combination with conjugated monoclonal antibodies, such as ibritumomab tiuxetan (Zevalin®), brentuximab vedotin (Adcetris®), ado-trastuzumab emtansine (Kadcyla®), or denileukin diftitox (Ontak®). In some embodiments, the peptidomimetic macrocycles of the disclosure are used in combination with bispecific monoclonal antibodies, such as blinatumomab (Blincyto®).

In some embodiments, the peptidomimetic macrocycles of the disclosure are used in combination with an anti-CD20 antibody, such as rituximab (Mabthera®/Rituxan®), obinutuzumab (Gazyva®), ibritumomab tiuxetan, tositumomab, ofatumumab (Genmab®), ocaratuzumab, ocrelizumab, TRU-015, or veltuzumab. Other antibodies that can be used in combination with the peptidomimetic macrocycles of the disclosure include antibodies against the programed cell death (PD-1) receptor, for example pembrolizumab (Keytruda®) or nivolumba (Opdivo®).

g. Combination Treatment with PD-L1 and/or PD-1 Antagonists

The PD-1 pathway comprises the immune cell co-receptor Programmed Death-1 (PD-1) and the PD-1 ligands PD-L1 and PD-L2. The PD-1 pathway mediates local immunosuppression in the tumor microenvironment. PD-1 and PD-L1 antagonists suppress the immune system. In some embodiments, a PD-1 or PD-L1 antagonist is a monoclonal antibody or antigen binding fragment thereof that specifically binds to, blocks, or downregulates PD-1 or PD-L1, respectively. In some embodiments, a PD-1 or PD-L1 antagonist is a compound or biological molecule that specifically binds to, blocks, or downregulates PD-1 or PD-L1, respectively.

In some embodiments, the peptidomimetic macrocycles of the disclosure are used in combination with a PD-1 or PD-L1 antagonist. In some embodiments, the peptidomimetic macrocycles of the disclosure are used in combination with a PD-1/PD-L1 antagonist, for example, MK-3475, nivolumab (Opdivo®), pembrolizumab (Keytruda®), humanized antibodies (i.e., h409A1 1, h409A16 and h409A17), AMP-514, BMS-936559, MEDI0680, MEDI4736, MPDL3280A, MSB0010718C, MDX-1105, MDX-1106, or pidilzumab. In some embodiments, the peptidomimetic macrocycles of the disclosure are used in combination with a PD-1/PD-L1 antagonist that is an immunoadhesion molecule, such as AMP-224. In some embodiments, the peptidomimetic macrocycles of the disclosure are used in combination with a PD-1/PD-L1 antagonist to treat cancer cells or a tumor that overexpresses PD-1 or PD-L1. In some embodiments, the peptidomimetic macrocycles of the disclosure are used in combination with a PD-1/PD-L1 antagonist to treat cancer cells or a tumor that overexpresses miR-34.

h. Combination Treatment with Anti-Hormone Therapy

Anti-hormone therapy uses an agent to suppress selected hormones or the effects. Anti-hormone therapy is achieved by antagonizing the function of hormones with a hormone antagonist and/or by preventing the production of hormones. In some embodiments, the suppression of hormones can be beneficial to subjects with certain cancers that grow in response to the presence of specific hormones. In some embodiments, the peptidomimetic macrocycles of the disclosure are used in combination with a hormone antagonist.

In some embodiments, the peptidomimetic macrocycles of the disclosure are used in combination with anti-androgens, anti-estrogens, aromatase inhibitors, or luteinizing hormone-releasing hormone (LHRH) agonists. In some embodiments, the peptidomimetic macrocycles of the disclosure are used in combination with anti-androgens, such as bicalutamide (Casodex®), cyproterone (Androcur®), flutamide (Euflex®), or nilutamide (Anandron®). In some embodiments, the peptidomimetic macrocycles of the disclosure are used in combination with anti-estrogens, such as fulvestrant (Faslodex®), raloxifene (Evista®), or tamoxifen (Novaladex®, Tamofen®). In some embodiments, the peptidomimetic macrocycles of the disclosure are used in combination with LHRH agonists, such as buserelin (Suprefact®), goserelin (Zoladex®), or leuprolide (Lupron®, Lupron Depot®, Eligard®).

i. Combination Treatment with Hypomethylating (Demethylating) Agents

Hypomethylating (demethylating) agents inhibit DNA methylation, which affects cellular function through successive generations of cells without changing the underlying DNA sequence. Hypomethylating agents can block the activity of DNA methyltransferase. In some embodiments, the peptidomimetic macrocycles of the disclosure can be used in combination with hypomethylating agents, such as azacitidine (Vidaza®, Azadine®) or decitabine (Dacogen®).

j. Combination Treatment with Anti-Inflammatory Agents

In some embodiments, the peptidomimetic macrocycles of the disclosure can be used in combination with nonsteroidal anti-inflammatory drugs (NSAIDs), specific COX-2 inhibitors, or corticosteroids. In some embodiments, the peptidomimetic macrocycles of the disclosure can be used in combination with NSAIDs, such as aspirin, ibuprofen, naproxen, celecoxib, ketorolac, or diclofenac. In some embodiments, the peptidomimetic macrocycles of the disclosure can be used in combination with specific COX-2 inhibitors, such as celecoxib (Celebrex®), rofecoxib, or etoricoxib. In some embodiments, the peptidomimetic macrocycles of the disclosure can be used in combination with corticosteroids, such as dexamethasone or glucosteroids (e.g., hydrocortisone and prednisone).

k. Combination Treatment with HDAC Inhibitors

Histone deacetylase (HDAC) inhibitors are chemical compounds that inhibit histone deacetylase. HDAC inhibitors can induce p21 expression, a regulator of p53 activity. In some embodiments, the peptidomimetic macrocycles of the disclosure can be used in combination with an HDAC inhibitor. In some embodiments, the peptidomimetic macrocycles of the disclosure can be used in combination with an HDAC inhibitor, such as vorinostat, romidepsin (Istodax®), chidamide, panobinostat (Farydak®), belinostat (PDX101), panobinostat (LBH589), valproic acid, mocetinostat (MGCD0103), abexinostat (PCI-24781), entinostat (MS-275), SB939, resminostat (4SC-201), givinostat (ITF2357), quisinostat (JNJ-26481585), HBI-8000, kevetrin, CUDC-101, AR-42, CHR-2845, CHR-3996, 4SC-202, CG200745, ACY-1215, ME-344, sulforaphane, or trichostatin A.

l. Combination Treatment with Platinum-Based Antineoplastic Drugs

Platinum-based antineoplastic drugs are coordinated complex of platinum. In some embodiments, the peptidomimetic macrocycles of the disclosure can be used in combination with a platinum-based antineoplastic drug, such as cisplatin, oxaliplatin, carboplatin, nedaplatin, triplatin tetranitrate, phenanthriplatin, picoplatin, or satraplatin. In some embodiments, the peptidomimetic macrocycles of the disclosure can be used in combination with cisplatin or carboplatin. In some embodiments, the peptidomimetic macrocycles of the disclosure can be used in combination with cisplatinum, platamin, neoplatin, cismaplat, cis-diamminedichloroplatinum(II), or CDDP; Platinol®) and carboplatin (also known as cis-diammine(1,1-cyclobutanedicarboxylato)platinum(II); tradenames Paraplatin® and Paraplatin-AQ®).

m. Combination Treatment with Kinase Inhibitors

Abnormal activation of protein phosphorylation is frequently either a driver of direct consequence of cancer. Kinase signaling pathways are involved in the phenotypes of tumor biology, including proliferation, survival, motility, metabolism, angiogenesis, and evasion of antitumor immune responses.

MEK Inhibitors:

MEK inhibitors are drugs that inhibit the mitogen-activated protein kinase enzymes MEK1 and/or MEK2. In some embodiments, the peptidomimetic macrocycles of the disclosure can be used in combination with a MEK1 inhibitor. In some embodiments, the peptidomimetic macrocycles of the disclosure can be used in combination with a MEK2 inhibitor. In some embodiments, the peptidomimetic macrocycles of the disclosure can be used in combination with an agent that can inhibit MEK1 and MEK2. In some embodiments, the peptidomimetic macrocycles of the disclosure can be used in combination with a MEK1/MEK2 inhibitor, such as trametinib (Mekinist®), cobimetinib, binimetinib, selumetinib (AZD6244), pimasertibe (AS-703026), PD-325901, CI-1040, PD035901, or TAK-733. In some embodiments, the peptidomimetic macrocycles of the disclosure are used in combination with trametinib. In some embodiments, the peptidomimetic macrocycles of the disclosure are used in combination with cobimetinib.

BRAF Inhibitors:

BRAF inhibitors are drugs that inhibit the serine/threonine-protein kinase B-raf (BRAF) protein. In some embodiments, the peptidomimetic macrocycles of the disclosure can be used in combination with a BRAF inhibitor. In some embodiments, the peptidomimetic macrocycles of the disclosure can be used in combination with a BRAF inhibitor that can inhibit wild type BRAF. In some embodiments, the peptidomimetic macrocycles of the disclosure can be used in combination with a BRAF inhibitor that can inhibit mutated BRAF. In some embodiments, the peptidomimetic macrocycles of the disclosure can be used in combination with a BRAF inhibitor that can inhibit V600E mutated BRAF. In some embodiments, the peptidomimetic macrocycles of the disclosure can be used in combination with a BRAF inhibitor, such as vemurafenib (Zelboraf®), dabrafenib (Tafinlar®), C-1, NVP-LGX818, or sorafenib (Nexavar®).

KRAS Inhibitors:

KRAS is a gene that acts as an on/off switch in cell signaling. In some embodiments, the peptidomimetic macrocycles of the disclosure can be used in combination with a KRAS inhibitor. In some embodiments, the peptidomimetic macrocycles of the disclosure can be used in combination with a wild type KRAS inhibitor. In some embodiments, the peptidomimetic macrocycles of the disclosure can be used in combination with a mutated KRAS inhibitor.

BTK Inhibitors:

Bruton's tyrosine kinase (BTK) is a non-receptor tyrosine kinase of the Tec kinase family that is involved in B-cell receptor signaling. In some embodiments, the peptidomimetic macrocycles of the disclosure can be used in combination with a BTK inhibitor. In some embodiments, the peptidomimetic macrocycles of the disclosure can be used in combination with a BTK inhibitor, such as ibrutinib or acalabrutinib.

CDK Inhibitors:

CDK4 and CDK6 are cyclin-dependent kinases that control the transition between the G1 and S phases of the cell cycle. CDK4/CDK6 activity is deregulated and overactive in cancer cells. Selective CDK4/CDK6 inhibitors can block cell-cycle progression in the mid-G1 phase of the cell cycle, causing arrest and preventing the proliferation of cancer cells. In some embodiments, the peptidomimetic macrocycles of the disclosure can be used in combination with a CDK4/CDK6 inhibitor. In some embodiments, the peptidomimetic macrocycles of the disclosure can be used in combination with a CDK4/CDK6 inhibitor, such as palbociclib (Ibrance®), ribociclib, trilaciclib, seliciclib, dinaciclib, milciclib, roniciclib, atuveciclib, briciclib, riviciclib, voruciclib, or abemaciclib. In some embodiments, the peptidomimetic macrocycles of the disclosure can be used in combination with palbociclib. In some embodiments, the peptidomimetic macrocycles of the disclosure can be used in combination with ribociclib. In some embodiments, the peptidomimetic macrocycles of the disclosure can be used in combination with abemaciclib.

In some examples, the peptidomimetic macrocycles of the disclosure may be used in combination with an inhibitor of CDK4 and/or CDK6 and with an agent that reinforces the cytostatic activity of CDK4/6 inhibitors and/or with an agent that converts reversible cytostasis into irreversible growth arrest or cell death. Exemplary cancer subtypes include NSCLC, melanoma, neuroblastoma, glioblastoma, liposarcoma, and mantle cell lymphoma. In some examples, the peptidomimetic macrocycles of the disclosure may also be used in combination with at least one additional pharmaceutically active agent that alleviates CDKN2A (cyclin-dependent kinase inhibitor 2A) deletion. In some example, the peptidomimetic macrocycles of the disclosure may also be used in combination with at least one additional pharmaceutically active agent that alleviates CDK9 (cyclin-dependent kinase 9) abnormality.

In some embodiments, the peptidomimetic macrocycles of the disclosure can be used in combination with a CDK2, CDK7, and/or CDK9 inhibitor. In some embodiments, the peptidomimetic macrocycles of the disclosure can be used in combination with a CDK2, CDK7, or CDK9 inhibitor, such as seliciclib, voruciclib, or milciclib. In some embodiments, the peptidomimetic macrocycles of the disclosure can be used in combination with a CDK inhibitor, such as dinaciclib, roniciclib (Kisqali®), or briciclib. In some examples, the peptidomimetic macrocycles of the disclosure may also be used in combination with at least one additional pharmaceutically-active agent that alleviates CDKN2A (cyclin-dependent kinase inhibitor 2A) deletion.

In some embodiments, a method of treating cancer in a subject in need thereof can comprise administering to the subject a therapeutically effective amount of a p53 agent that inhibits the interaction between p53 and MDM2 and/or p53 and MDMX, and/or modulates the activity of p53 and/or MDM2 and/or MDMX; and at least one additional pharmaceutically-active agent, wherein the at least one additional pharmaceutically-active agent modulates the activity of CDK4 and/or CDK6, and/or inhibits CDK4 and/or CDK6.

ATM Regulators:

The peptidomimetic macrocycles of the disclosure may also be used in combination with one or more pharmaceutically-active agent that regulates the ATM (upregulate or downregulate). In some embodiments the compounds described herein can synergize with one or more ATM regulators. In some embodiments one or more of the compounds described herein can synergize with all ATM regulators.

AKT Inhibitors:

In some embodiments, the peptidomimetic macrocycles of the disclosure may be used in combination with one or more pharmaceutically-active agent that inhibits the AKT (protein kinase B (PKB)). In some embodiments the compounds described herein can synergize with one or more AKT inhibitors.

n. Combination Treatment with Other Pharmaceutically-Active Agents

In some examples, the peptidomimetic macrocycles of the disclosure may also be used in combination with at least one additional pharmaceutically-active agent that alleviates PTEN (phosphatase and tensin homolog) deletion.

In some examples, the peptidomimetic macrocycles of the disclosure may also be used in combination with at least one additional pharmaceutically-active agent that alleviates Wip-1Alpha over expression.

In some examples, the peptidomimetic macrocycles of the disclosure may be used in combination with at least one additional pharmaceutically-active agent that is a Nucleoside metabolic inhibitor. Exemplary nucleoside metabolic inhibitors that may be used include capecitabine, gemcitabine and cytarabine (Arac).

The table below lists suitable additional pharmaceutically-active agents for use with the methods described herein.

| | | | Drug works predominately |
|---|---|---|---|
| Cancer Type | Drug name | Brand name | in S or M phase |
| ALL | ABT-199 | none | No |
| ALL | clofarabine | Clofarex | Yes; S phase |
| ALL | cyclophosphamide | Clafen, Cytoxan, Neosar | Yes: S phase |
| ALL | cytarabine | Cytosar-U, Tarabine PFS | Yes: S phase |
| ALL | doxorubicin | Adriamycin | Yes: S phase |
| ALL | imatinib mesylate | Gleevec | No |
| ALL | methotrexate | Abitrexate, Mexate, Folex | Yes: S phase |
| ALL | prednisone | Deltasone, Medicorten | No |
| ALL | romidepsin | Istodax | |
| ALL | vincristine | Vincasar | Yes: M phase |
| AML | ABT-199 | none | No |
| AML | azacitadine | Vidaza | No |
| AML | cyclophosphamide | Clafen, Cytoxan, Neosar | Yes: S phase |
| AML | cytarabine | Cytosar-U, Tarabine PFS | Yes: S phase |
| AML | decitabine | Dacogen | No |
| AML | doxorubicin | Adriamycin | Yes: S phase |
| AML | etoposide | Etopophos, Vepesid | Yes: S and M phases |
| AML | vincristine | Vincasar | Yes: M phase |
| bone | doxorubicin | Adriamycin | Yes: S phase |
| bone | methotrexate | Abitrexate, Mexate, Folex | Yes: S phase |
| breast | capecitabine | Xeloda | Yes: S phase |
| breast | cyclophosphamide | Clafen, Cytoxan, Neosar | Yes: S phase |
| breast | docetaxel | Taxotere | Yes: M phase |
| breast | doxorubicin | Adriamycin | Yes: S phase |
| breast | eribulin mesylate | Haliben | Yes: M phase |
| breast | everolimus | Afinitor | No |
| breast | exemestane | Aromasin | No |
| breast | fluorouracil | Adrucil, Efudex | Yes: S phase |
| breast | fulvestrant | Faslofex | |
| breast | gemcitabine | Gemzar | Yes: S phase |
| breast | goserelin acetate | Zoladex | No |
| breast | letrozole | Femara | No |
| breast | megestrol acetate | Megace | No |
| breast | methotrexate | Abitrexate, Mexate, Folex | Yes: S phase |
| breast | paclitaxel | Abraxane ®, Taxol | Yes: M phase |
| breast | palbociclib | Ibrance | Might cause G1 arrest |
| breast | pertuzumab | Perjeta | No |
| breast | tamoxifen citrate | Nolvadex | No |
| breast | trastuzumab | Herceptin, Kadcyla | No |
| colon | capecitabine | Xeloda | Yes: S phase |
| colon | cetuximab | Erbitux | No |
| colon | fluorouracil | Adrucil, Efudex | Yes: S phase |
| colon | irinotecan | camptosar | Yes: S and M phases |
| colon | ramucirumab | Cyramza | No |
| endometrial | carboplatin | Paraplatin, Paraplat | Yes: S phase |
| endometrial | cisplatin | Platinol | Yes: S phase |
| endometrial | doxorubicin | Adriamycin | Yes: S phase |
| endometrial | megestrol acetate | Megace | No |
| endometrial | paclitaxel | Abraxane ®, Taxol | Yes: M phase |
| gastric | docetaxel | Taxotere | Yes: M phase |
| gastric | doxorubicin | Adriamycin | Yes: S phase |
| gastric | fluorouracil | Adrucil, Efudex | Yes: S phase |
| gastric | ramucirumab | Cyramza | No |
| gastric | trastuzumab | Herceptin | No |
| kidney | axitinib | Inlyta | No |
| kidney | everolimus | Afinitor | No |
| kidney | pazopanib | Votrient | No |
| kidney | sorafenib tosylate | Nexavar | No |
| liver | sorafenib tosylate | Nexavar | No |
| melanoma | dacarbazine | DTIC, DTIC-Dome | Yes: S phase |
| melanoma | paclitaxel | Abraxane ®, Taxol | Yes: M phase |
| melanoma | trametinib | Mekinist | No |
| melanoma | vemurafenib | Zelboraf | No |
| melanoma | dabrafenib | Taflinar | |
| mesothelioma | cisplatin | Platinol | Yes: S phase |
| mesothelioma | pemetrexed | Alimta | Yes: S phase |
| NHL | ABT-199 | none | No |
| NHL | bendamustine | Treanda | Causes DNA crosslinking, |
| | | | but is also toxic to resting |
| | | | cells |
| NHL | bortezomib | Velcade | No |
| NHL | brentuximab vedotin | Adcetris | Yes: M phase |
| NHL | chlorambucil | Ambochlorin, Leukeran, Linfolizin | Yes: S phase |
| NHL | cyclophosphamide | Clafen, Cytoxan, Neosar | Yes: S phase |
| NHL | dexamethasone | Decadrone, Dexasone | No |
| NHL | doxorubicin | Adriamycin | Yes: S phase |
| NHL | Ibrutinib | Imbruvica | No |
| NHL | lenalidomide | Revlimid | No |
| NHL | methotrexate | Abitrexate, Mexate, Folex | Yes: S phase |
| NHL | obinutuzumab | Gazyva | No |
| NHL | prednisone | Deltasone, Medicorten | No |
| NHL | romidepsin | Istodax | |
| NHL | rituximab | Rituxan | No |
| NHL | vincristine | Vincasar | Yes: M phase |
| NSCLC | afatinib Dimaleate | Gilotrif | No |
| NSCLC | carboplatin | Paraplatin, Paraplat | Yes: S phase |
| NSCLC | cisplatin | Platinol | Yes: S phase |
| NSCLC | crizotinib | Xalkori | No |
| NSCLC | docetaxel | Taxotere | Yes: M phase |
| NSCLC | erlotinib | Tarceva | No |
| NSCLC | gemcitabine | Gemzar | Yes: S phase |
| NSCLC | methotrexate | Abitrexate, Mexate, Folex | Yes: S phase |
| NSCLC | paclitaxel | Abraxane ®, Taxol | Yes: M phase |
| NSCLC | palbociclib | Ibrance | Might cause G1 arrest |
| NSCLC | pemetrexed | Alimta | Yes: S phase |
| NSCLC | ramucirumab | Cyramza | No |
| ovarian | carboplatin | Paraplatin, Paraplat | Yes: S phase |
| ovarian | cisplatin | Platinol | Yes; S phase |
| ovarian | cyclophosphamide | Clafen, Cytoxan, Neosar | Yes: S phase |
| ovarian | gemcitabine | Gemzar | Yes: S phase |
| ovarian | olaparib | Lynparza | Yes: G2/M phase arrest |
| ovarian | paclitaxel | Abraxane ®, Taxol | Yes: M phase |
| ovarian | topotecan | Hycamtin | Yes: S phase |
| prostate | abiraterone | Zytiga | No |
| prostate | cabazitaxel | Jevtana | Yes: M phase |
| prostate | docetaxel | Taxotere | Yes: M phase |
| prostate | enzalutamide | Xtandi | No |
| prostate | goserelin acetate | Zoladex | No |
| prostate | prednisone | Deltasone, Medicorten | No |
| soft tissue sarcoma | doxorubicin | Adriamycin | Yes: S phase |
| soft tissue sarcoma | imatinib mesylate | Gleevec | No |
| soft tissue sarcoma | pazopanib | Votrient | No |
| T-cell lymphoma | romidepsin | Istodax | |

### Administration of Combination Treatment

The peptidomimetic macrocycles or a composition comprising same and the at least one additional pharmaceutically-active agent, for example, any additional therapeutic agent described herein, or a composition comprising same can be administered simultaneously (i.e., simultaneous administration) and/or sequentially (i.e., sequential administration).

According to certain embodiments, the peptidomimetic macrocycles and the at least one additional pharmaceutically-active agent, for example, any additional therapeutic agent described herein, are administered simultaneously, either in the same composition or in separate compositions. The term “simultaneous administration,” as used herein, means that the peptidomimetic macrocycle and the at least one additional pharmaceutically-active agent, for example, any additional therapeutic agent described herein, are administered with a time separation of no more than a few minutes, for example, less than about 15 minutes, less than about 10, less than about 5, or less than about 1 minute. When the drugs are administered simultaneously, the peptidomimetic macrocycle and the at least one additional pharmaceutically-active agent, for example, any additional therapeutic agent described herein, may be contained in the same composition (e.g., a composition comprising both the peptidomimetic macrocycle and the at least additional pharmaceutically-active agent) or in separate compositions (e.g., the peptidomimetic macrocycle is contained in one composition and the at least additional pharmaceutically-active agent is contained in another composition).

According to other embodiments, the peptidomimetic macrocycles and the at least one additional pharmaceutically-active agent, for example, any additional therapeutic agent described herein, are administered sequentially, i.e., the peptidomimetic macrocycle is administered either prior to or after the administration of the additional pharmaceutically-active agent. The term “sequential administration” as used herein means that the peptidomimetic macrocycle and the additional pharmaceutically-active agent, for example, any additional therapeutic agent described herein, are administered with a time separation of more than a few minutes, for example, more than about 15 minutes, more than about 20 or more minutes, more than about 30 or more minutes, more than about 40 or more minutes, more than about 50 or more minutes, or more than about 60 or more minutes. In some embodiments, the peptidomimetic macrocycle is administered before the additional pharmaceutically-active agent, for example, any additional therapeutic agent described herein. In some embodiments, the pharmaceutically-active agent, for example, any additional therapeutic agent described herein, is administered before the peptidomimetic macrocycle. The peptidomimetic macrocycle and the additional pharmaceutically-active agent, for example, any additional therapeutic agent described herein, are contained in separate compositions, which may be contained in the same or different packages.

In some embodiments, the administration of the peptidomimetic macrocycles and the additional pharmaceutically-active agent, for example, any additional therapeutic agent described herein, are concurrent, i.e., the administration period of the peptidomimetic macrocycles and that of the agent overlap with each other. In some embodiments, the administration of the peptidomimetic macrocycles and the additional pharmaceutically-active agent, for example, any additional therapeutic agent described herein, are non-concurrent. For example, in some embodiments, the administration of the peptidomimetic macrocycles is terminated before the additional pharmaceutically-active agent, for example, any additional therapeutic agent described herein, is administered. In some embodiments, the administration of the additional pharmaceutically-active agent, for example, any additional therapeutic agent described herein, is terminated before the peptidomimetic macrocycle is administered. The time period between these two non-concurrent administrations can range from being days apart to being weeks apart.

The dosing frequency of the peptidomimetic macrocycle and the at least one additional pharmaceutically-active agent, for example, any additional therapeutic agent described herein, may be adjusted over the course of the treatment, based on the judgment of the administering physician. When administered separately, the peptidomimetic macrocycle and the at least one additional pharmaceutically-active agent, for example, any additional therapeutic agent described herein, can be administered at different dosing frequency or intervals. For example, the peptidomimetic macrocycle can be administered weekly, while the at least one additional pharmaceutically-active agent, for example, any additional therapeutic agent described herein, can be administered more or less frequently. Or, the peptidomimetic macrocycle can be administered twice weekly, while the at least one additional pharmaceutically-active agent, for example, any additional therapeutic agent described herein, can be administered more or less frequently. In addition, the peptidomimetic macrocycle and the at least one additional pharmaceutically-active agent, for example, any additional therapeutic agent described herein, can be administered using the same route of administration or using different routes of administration.

A therapeutically effective amount of a peptidomimetic macrocycle and/or the additional pharmaceutically-active agent, for example, any additional therapeutic agent described herein, for use in therapy can vary with the nature of the condition being treated, the length of treatment time desired, the age and the condition of the patient, and can be determined by the attending physician. Doses employed for human treatment can be in the range of about 0.01 mg/kg to about 1000 mg/kg per day (e.g., about 0.01 mg/kg to about 100 mg/kg per day, about 0.01 mg/kg to about 10 mg/kg per day, about 0.1 mg/kg to about 100 mg/kg per day, about 0.1 mg/kg to about 50 mg/kg per day, about 0.1 mg/kg to about 10 mg/kg per day) of one or each component of the combinations described herein. In some embodiments, doses of a peptidomimetic macrocycle employed for human treatment are in the range of about 0.01 mg/kg to about 100 mg/kg per day (e.g., about 0.01 mg/kg to about 10 mg/kg per day, about 0.1 mg/kg to about 100 mg/kg per day, about 0.1 mg/kg to about 50 mg/kg per day, about 0.1 mg/kg to about 10 mg/kg per day, about 1 mg/kg per day). In some embodiments, doses of the additional pharmaceutically-active agent, for example, any additional therapeutic agent described herein, employed for human treatment can be in the range of about 0.01 mg/kg to about 100 mg/kg per day (e.g., about 0.1 mg/kg to about 100 mg/kg per day, about 0.1 mg/kg to about 50 mg/kg per day, about 10 mg/kg per day or about 30 mg/kg per day). The desired dose may be conveniently administered in a single dose, or as multiple doses administered at appropriate intervals, for example as two, three, four or more sub-doses per day.

In some embodiments, such as when given in combination with the at least one additional pharmaceutically active agent, for example, any additional therapeutic agent described herein, the dosage of a peptidomimetic macrocycle may be given at relatively lower dosages. In some embodiments, the dosage of a peptidomimetic macrocycle may be from about 1 ng/kg to about 100 mg/kg. The dosage of a peptidomimetic macrocycle may be at any dosage including, but not limited to, about 1 μg/kg, 25 μg/kg, 50 μg/kg, 75 μg/kg, 100 i g/kg, 125 μg/kg, 150 μg/kg, 175 μg/kg, 200 μg/kg, 225 μg/kg, 250 μg/kg, 275 μg/kg, 300 μg/kg, 325 μg/kg, 350 μg/kg, 375 μg/kg, 400 μg/kg, 425 μg/kg, 450 μg/kg, 475 μg/kg, 500 μg/kg, 525 μg/kg, 550 μg/kg, 575 μg/kg, 600 μg/kg, 625 μg/kg, 650 μg/kg, 675 μg/kg, 700 μg/kg, 725 μg/kg, 750 μg/kg, 775 μg/kg, 800 μg/kg, 825 μg/kg, 850 μg/kg, 875 μg/kg, 900 μg/kg, 925 μg/kg, 950 μg/kg, 975 μg/kg, 1 mg/kg, 2.5 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, or 100 mg/kg.

In some embodiments, the dosage of the additional pharmaceutically-active agent, for example, any additional therapeutic agent described herein, may be from about 1 ng/kg to about 100 mg/kg. The dosage of the additional pharmaceutically-active agent may be at any dosage including, but not limited to, about 1 μg/kg, 25 μg/kg, 50 μg/kg, 75 μg/kg, 100 i g/kg, 125 μg/kg, 150 μg/kg, 175 μg/kg, 200 μg/kg, 225 μg/kg, 250 μg/kg, 275 μg/kg, 300 μg/kg, 325 μg/kg, 350 μg/kg, 375 μg/kg, 400 μg/kg, 425 μg/kg, 450 μg/kg, 475 μg/kg, 500 μg/kg, 525 μg/kg, 550 μg/kg, 575 μg/kg, 600 μg/kg, 625 μg/kg, 650 μg/kg, 675 μg/kg, 700 μg/kg, 725 μg/kg, 750 μg/kg, 775 μg/kg, 800 μg/kg, 825 μg/kg, 850 μg/kg, 875 μg/kg, 900 μg/kg, 925 μg/kg, 950 μg/kg, 975 μg/kg, 1 mg/kg, 2.5 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, or 100 mg/kg.

In some embodiments, the dosage of the additional pharmaceutically-active agent is the approved dosage from the label of the additional pharmaceutically-active agent. In some embodiments, the dosage of the additional pharmaceutically-active agent is 600 mg of ribociclib; 150 mg or 200 mg of abemaciclib; 125 mg of palbociclib; 2 mg of trametinib; 175 mg/m², 135 mg/m², or 100 mg/m² of paclitaxel; 1.4 mg/m² of eribulin; 250 mg/m² (breast cancer), 100 mg/m² (non-small cell lung cancer), or 125 mg/m² (pancreatic cancer) of Abraxane®; 200 mg of Keytruda®; or 240 mg or 480 mg of Opdivo®, or a pharmaceutically-acceptable salt of any of the foregoing. In some embodiments, the approved dosages of the additional pharmaceutically-active agents can be reduced to address adverse side effects such as renal impairment or liver impairment.

The peptidomimetic macrocycle and the additional pharmaceutically-active agent, for example, any additional therapeutic agent described herein, can be provided in a single unit dosage form for being taken together or as separate entities (e.g. in separate containers) to be administered simultaneously or with a certain time difference. This time difference may be between 1 hour and 1 month, e.g., between 1 day and 1 week, e.g., 48 hours and 3 days. In addition, it is possible to administer the peptidomimetic macrocycle via another administration way than the additional pharmaceutically-active agent, for example, any additional therapeutic agent described herein. For example, it may be advantageous to administer either the peptidomimetic macrocycle or the additional pharmaceutically-active agent, for example, any additional therapeutic agent described herein, intravenously and the other systemically or orally. For example, the peptidomimetic macrocycle is administered intravenously and the additional pharmaceutically-active agent orally.

In some embodiments, the peptidomimetic macrocycle is administered about 0.1 hour, 0.2 hour, 0.3 hour, 0.4 hour, 0.5 hour, 0.6 hour, 0.7 hour, 0.8 hour, 0.9 hour, 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 month, 2 months, 3 months, 4 months, 5 months, 6 months, 7 months, 8 months, 9 months, 10 months, 11 months, or 12 months before the additional pharmaceutically-active agent, for example, any additional therapeutic agent described herein, is administered. In some embodiments, the peptidomimetic macrocycle is administered about 6 hours before the additional pharmaceutically-active agent, for example, any additional therapeutic agent described herein, is administered.

In some embodiments, the peptidomimetic macrocycle is administered about 0.1 hour, 0.2 hour, 0.3 hour, 0.4 hour, 0.5 hour, 0.6 hour, 0.7 hour, 0.8 hour, 0.9 hour, 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 month, 2 months, 3 months, 4 months, 5 months, 6 months, 7 months, 8 months, 9 months, 10 months, 11 months, or 12 months after the additional pharmaceutically-active agent, for example, any additional therapeutic agent described herein, is administered. In some embodiments, the peptidomimetic macrocycle is administered about 6 hours after the additional pharmaceutically-active agent, for example, any additional therapeutic agent described herein, is administered.

In some embodiments, the peptidomimetic macrocycle is administered chronologically before the additional pharmaceutically-active agent, for example, any additional therapeutic agent described herein. In some embodiments, the peptidomimetic macrocycle is administered from 1-24 hours, 2-24 hours, 3-24 hours, 4-24 hours, 5-24 hours, 6-24 hours, 7-24 hours, 8-24 hours, 9-24 hours, 10-24 hours, 11-24 hours, 12-24 hours, 1-30 days, 2-30 days, 3-30 days, 4-30 days, 5-30 days, 6-30 days, 7-30 days, 8-30 days, 9-30 days, 10-30 days, 11-30 days, 12-30 days, 13-30 days, 14-30 days, 15-30 days, 16-30 days, 17-30 days, 18-30 days, 19-30 days, 20-30 days, 21-30 days, 22-30 days, 23-30 days, 24-30 days, 25-30 days, 26-30 days, 27-30 days, 28-30 days, 29-30 days, 1-4 week, 2-4 weeks, 3-4 weeks, 1-12 months, 2-12 months, 3-12 months, 4-12 months, 5-12 months, 6-12 months, 7-12 months, 8-12 months, 9-12 months, 10-12 months, 11-12 months, or any combination thereof, before the additional pharmaceutically-active agent, for example, any additional therapeutic agent described herein, is administered. In some embodiments, the peptidomimetic macrocycle is administered at least 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before the additional pharmaceutically-active agent, for example, any additional therapeutic agent described herein, is administered. For example, the peptidomimetic macrocycle can be administered at least 6 hours before a CDKI (e.g., seliciclib, ribociclib, abemaciclib, or palbociclib) is administered.

In some embodiments, the peptidomimetic macrocycle is administered at most 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before the additional pharmaceutically-active agent is administered. For example, the peptidomimetic macrocycle can be administered at most 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before a CDKI (e.g., seliciclib, ribociclib, abemaciclib, or palbociclib) is administered.

In some embodiments, the peptidomimetic macrocycle is administered about 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before the additional pharmaceutically-active agent, for example, any additional therapeutic agent described herein, is administered. For example, the peptidomimetic macrocycle can be administered about 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before a CDKI (e.g., seliciclib, ribociclib, abemaciclib, or palbociclib) is administered.

In some embodiments, the peptidomimetic macrocycle is administered chronologically at the same time as the at least one additional pharmaceutically active agent, for example, any additional therapeutic agent described herein.

In some embodiments, the peptidomimetic macrocycle is administered chronologically after the additional pharmaceutically-active agent, for example, any additional therapeutic agent described herein. In some embodiments, the additional pharmaceutically-active agent, for example, any additional therapeutic agent described herein, is administered from 1-24 hours, 2-24 hours, 3-24 hours, 4-24 hours, 5-24 hours, 6-24 hours, 7-24 hours, 8-24 hours, 9-24 hours, 10-24 hours, 11-24 hours, 12-24 hours, 1-30 days, 2-30 days, 3-30 days, 4-30 days, 5-30 days, 6-30 days, 7-30 days, 8-30 days, 9-30 days, 10-30 days, 11-30 days, 12-30 days, 13-30 days, 14-30 days, 15-30 days, 16-30 days, 17-30 days, 18-30 days, 19-30 days, 20-30 days, 21-30 days, 22-30 days, 23-30 days, 24-30 days, 25-30 days, 26-30 days, 27-30 days, 28-30 days, 29-30 days, 1-4 week, 2-4 weeks, 3-4 weeks, 1-12 months, 2-12 months, 3-12 months, 4-12 months, 5-12 months, 6-12 months, 7-12 months, 8-12 months, 9-12 months, 10-12 months, 11-12 months, or any combination thereof, before the peptidomimetic macrocycle is administered. In some embodiments the additional pharmaceutically-active agent is administered at least 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before the peptidomimetic macrocycle is administered. For example, seliciclib, ribociclib, abemaciclib, or palbociclib can be administered at least 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 week, 2 weeks, 3 weeks, 4 weeks, 1 month, 2 months, 3 months, 4 months, 5 months, 6 months, 7 months, 8 months, 9 months, 10 months, 11 months, 12 months, or any combination thereof, before the peptidomimetic macrocycle is administered.

In some embodiments, a CDKI is administered at most 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before the peptidomimetic macrocycle is administered. For example, seliciclib, ribociclib, abemaciclib, or palbociclib can be administered at most 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 week, 2 weeks, 3 weeks, 4 weeks, 1 month, 2 months, 3 months, 4 months, 5 months, 6 months, 7 months, 8 months, 9 months, 10 months, 11 months, 12 months, or any combination thereof, before the peptidomimetic macrocycle is administered.

In some embodiments a CDKI is administered about 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before the peptidomimetic macrocycle is administered. For example, seliciclib, ribociclib, abemaciclib, or palbociclib can be administered about 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 week, 2 weeks, 3 weeks, 4 weeks, 1 month, 2 months, 3 months, 4 months, 5 months, 6 months, 7 months, 8 months, 9 months, 10 months, 11 months, 12 months, or any combination thereof, before the peptidomimetic macrocycle is administered.

Also, contemplated herein is a drug holiday utilized among the administration of a peptidomimetic macrocycle and an additional pharmaceutically-active agent, for example, any additional therapeutic agent described herein. A drug holiday can be a period of days after the administration of the additional pharmaceutically-active agent, for example, any additional therapeutic agent described herein, and before the administration of a peptidomimetic macrocycle. A drug holiday can be a period of days after the administration of a peptidomimetic macrocycle and before the administration of the additional pharmaceutically-active agent, for example, any additional therapeutic agent described herein. A drug holiday can be a period of days after the sequential administration of one or more of a peptidomimetic macrocycle and an additional pharmaceutically-active agent, for example, any additional therapeutic agent described herein, and before the administration of the peptidomimetic macrocycle, the additional pharmaceutically-active agent or another therapeutic agent. For example, a drug holiday can be a period of days after the sequential administration of a peptidomimetic macrocycle first, followed administration of an additional pharmaceutically-active agent, for example, any additional therapeutic agent described herein, and before the administration of the peptidomimetic macrocycle again. For example, a drug holiday can be a period of days after the sequential administration of an additional pharmaceutically-active agent first, followed administration of a peptidomimetic macrocycle and before the administration of the additional pharmaceutically-active agent, for example, any additional therapeutic agent described herein.

Suitably the drug holiday will be a period of 1 day, 2 days, 3 days, 4 days, 5 days, 6 days, 7 days, 8 days, 9 days, 10 days, 11 days, 12 days, 13 days or 14 days; or from 1-24, 2-24, 3-24, 4-24, 5-24, 6-24, 7-24, 8-24, 9-24, 10-24, 11-24, or 12-24 hours; from 1-30, 2-30, 3-30, 4-30, 5-30, 6-30, 7-30, 8-30, 9-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 days, 1-4, 2-4, or 3-4 weeks; or from 1-12, 2-12, 3-12, 4-12, 5-12, 6-12, 7-12, 8-12, 9-12, 10-12, or 11-12 months.

In some embodiments, an additional pharmaceutically-active agent, for example, any additional therapeutic agent described herein, will be administered first in the sequence, followed by an optional drug holiday, followed by administration of a peptidomimetic macrocycle. In some embodiments, an additional pharmaceutically-active agent, for example, any additional therapeutic agent described herein, will be administered first in the sequence, followed by administration of a peptidomimetic macrocycle, followed by an optional drug holiday, followed by administration of an additional pharmaceutically-active agent.

In some embodiments, an additional pharmaceutically-active agent, for example, any additional therapeutic agent described herein, is administered for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4, or 3-4 consecutive weeks; or from 1-12, 2-12, 3-12, 4-12, 5-12, 6-12, 7-12, 8-12, 9-12, 10-12, or 11-12 consecutive months, followed by an optional drug holiday; followed by administration of a peptidomimetic macrocycle for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4, or 3-4 consecutive weeks; or from 1-12, 2-12, 3-12, 4-12, 5-12, 6-12, 7-12, 8-12, 9-12, 10-12, or 11-12 consecutive months. For example, a cyclin dependent kinase inhibitor is administered for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4, or 3-4 consecutive weeks; or from 1-12, 2-12, 3-12, 4-12, 5-12, 6-12, 7-12, 8-12, 9-12, 10-12, or 11-12 consecutive months; followed by a drug holiday of from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4, or 3-4 consecutive weeks; or from 1-12, 2-12, 3-12, 4-12, 5-12, 6-12, 7-12, 8-12, 9-12, 10-12, or 11-12 consecutive months; followed by administration of a peptidomimetic macrocycle for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4, or 3-4 consecutive weeks; or from 1-12, 2-12, 3-12, 4-12, 5-12, 6-12, 7-12, 8-12, 9-12, 10-12, or 11-12 consecutive months.

In some embodiments, an additional pharmaceutically-active agent, for example, any additional therapeutic agent described herein, is administered for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4, or 3-4 consecutive weeks; or from 1-12, 2-12, 3-12, 4-12, 5-12, 6-12, 7-12, 8-12, 9-12, 10-12, or 11-12 consecutive months, followed by administration of a peptidomimetic macrocycle for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4, or 3-4 consecutive weeks; or from 1-12, 2-12, 3-12, 4-12, 5-12, 6-12, 7-12, 8-12, 9-12, 10-12, or 11-12 consecutive months, followed by an optional drug holiday; followed by administration of an additional pharmaceutically-active agent. For example, a cyclin dependent kinase inhibitor is administered for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4, or 3-4 consecutive weeks; or from 1-12, 2-12, 3-12, 4-12, 5-12, 6-12, 7-12, 8-12, 9-12, 10-12, or 11-12 consecutive months; followed by administration of a peptidomimetic macrocycle for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4, or 3-4 consecutive weeks; or from 1-12, 2-12, 3-12, 4-12, 5-12, 6-12, 7-12, 8-12, 9-12, 10-12, or 11-12 consecutive months, followed by an optional drug holiday of from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4, or 3-4 consecutive weeks; or from 1-12, 2-12, 3-12, 4-12, 5-12, 6-12, 7-12, 8-12, 9-12, 10-12, or 11-12 consecutive months; followed by administration of a cyclin dependent kinase inhibitor.

In some embodiments, a peptidomimetic macrocycle will be administered first in the sequence, followed by an optional drug holiday, followed by administration of an additional pharmaceutically-active agent, for example, any additional therapeutic agent described herein. In some embodiments, a peptidomimetic macrocycle will be administered first in the sequence, followed by administration of an additional pharmaceutically-active agent, for example, any additional therapeutic agent described herein, followed by an optional drug holiday, followed by administration of a peptidomimetic macrocycle.

In some embodiments, a peptidomimetic macrocycle is administered for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4, or 3-4 consecutive weeks; or from 1-12, 2-12, 3-12, 4-12, 5-12, 6-12, 7-12, 8-12, 9-12, 10-12, or 11-12 consecutive months, followed by an optional drug holiday; followed by administration of an additional pharmaceutically-active agent, for example, any additional therapeutic agent described herein, for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4, or 3-4 consecutive weeks; or from 1-12, 2-12, 3-12, 4-12, 5-12, 6-12, 7-12, 8-12, 9-12, 10-12, or 11-12 consecutive months. For example, a peptidomimetic macrocycle is administered for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4, or 3-4 consecutive weeks; or from 1-12, 2-12, 3-12, 4-12, 5-12, 6-12, 7-12, 8-12, 9-12, 10-12, or 11-12 consecutive months; followed by a drug holiday of from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4, or 3-4 consecutive weeks; or from 1-12, 2-12, 3-12, 4-12, 5-12, 6-12, 7-12, 8-12, 9-12, 10-12, or 11-12 consecutive months; followed by administration of a cyclin dependent kinase inhibitor for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4, or 3-4 consecutive weeks; or from 1-12, 2-12, 3-12, 4-12, 5-12, 6-12, 7-12, 8-12, 9-12, 10-12, or 11-12 consecutive months

In some embodiments, a peptidomimetic macrocycle is administered for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4, or 3-4 consecutive weeks; or from 1-12, 2-12, 3-12, 4-12, 5-12, 6-12, 7-12, 8-12, 9-12, 10-12, or 11-12 consecutive months, followed by administration of an additional pharmaceutically-active agent, for example, any additional therapeutic agent described herein, for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4, or 3-4 consecutive weeks; or from 1-12, 2-12, 3-12, 4-12, 5-12, 6-12, 7-12, 8-12, 9-12, 10-12, or 11-12 consecutive months, followed by an optional drug holiday; followed by administration of a peptidomimetic macrocycle. For example, a peptidomimetic macrocycle is administered for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4, or 3-4 consecutive weeks; or from 1-12, 2-12, 3-12, 4-12, 5-12, 6-12, 7-12, 8-12, 9-12, 10-12, or 11-12 consecutive months; followed by administration of a cyclin dependent kinase inhibitor for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4, or 3-4 consecutive weeks; or from 1-12, 2-12, 3-12, 4-12, 5-12, 6-12, 7-12, 8-12, 9-12, 10-12, or 11-12 consecutive months, followed by an optional drug holiday of from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4, or 3-4 consecutive weeks; or from 1-12, 2-12, 3-12, 4-12, 5-12, 6-12, 7-12, 8-12, 9-12, 10-12, or 11-12 consecutive months; followed by administration of a peptidomimetic macrocycle.

In some embodiments, an additional pharmaceutically-active agent, for example, any additional therapeutic agent described herein, will be administered first in the sequence, followed by an optional drug holiday, followed by administration of a peptidomimetic macrocycle. In some embodiments, a cyclin dependent kinase inhibitor will be administered first in the sequence, followed by an optional drug holiday, followed by administration of a peptidomimetic macrocycle, followed by an optional drug holiday, followed by administration of a cyclin dependent kinase inhibitor.

In some embodiments, an additional pharmaceutically-active agent, for example, any additional therapeutic agent described herein, is administered for from 1 to 30 consecutive days, followed by an optional drug holiday, followed by administration of peptidomimetic macrocycle for from 1 to 30 consecutive days. In some embodiments, an additional pharmaceutically-active agent, for example, any additional therapeutic agent described herein, is administered for from 1 to 21 consecutive days, followed by an optional drug holiday, followed by administration of a peptidomimetic macrocycle for from 1 to 21 consecutive days. In some embodiments, an additional pharmaceutically-active agent, for example, any additional therapeutic agent described herein, is administered for from 1 to 14 consecutive days, followed by an optional drug holiday, followed by administration of a peptidomimetic macrocycle for from 1 to 14 consecutive days. In some embodiments, an additional pharmaceutically-active agent, for example, any additional therapeutic agent described herein, is administered for 14 consecutive days, followed by an optional drug holiday, followed by administration of a peptidomimetic macrocycle for 7 consecutive days. In some embodiments, an additional pharmaceutically-active agent, for example, any additional therapeutic agent described herein, is administered for 7 consecutive days, followed by an optional drug holiday, followed by administration of a peptidomimetic macrocycle for 7 consecutive days.

In some embodiments, a peptidomimetic macrocycle is administered for from 1 to 30 consecutive days, followed by an optional drug holiday, followed by administration of an additional pharmaceutically-active agent, for example, any additional therapeutic agent described herein, for from 1 to 30 consecutive days. In some embodiments, a peptidomimetic macrocycle is administered for from 1 to 21 consecutive days, followed by an optional drug holiday, followed by administration of an additional pharmaceutically-active agent, for example, any additional therapeutic agent described herein, for from 1 to 21 consecutive days. In some embodiments, a peptidomimetic macrocycle is administered for from 1 to 14 consecutive days, followed by an optional drug holiday, followed by administration of an additional pharmaceutically-active agent, for example, any additional therapeutic agent described herein, for from 1 to 14 consecutive days. In some embodiments, a peptidomimetic macrocycle is administered for 14 consecutive days, followed by an optional drug holiday, followed by administration of an additional pharmaceutically-active agent, for example, any additional therapeutic agent described herein, for 14 consecutive days. In some embodiments, a peptidomimetic macrocycle is administered for 7 consecutive days, followed by an optional drug holiday, followed by administration of an additional pharmaceutically-active agent, for example, any additional therapeutic agent described herein, for 7 consecutive days.

In some embodiments, one of a peptidomimetic macrocycle and an additional pharmaceutically-active agent, for example, any additional therapeutic agent described herein, is administered for from 2 to 30 consecutive days, followed by an optional drug holiday, followed by administration of the other of a peptidomimetic macrocycle and an additional pharmaceutically-active agent for from 2 to 30 consecutive days. In some embodiments, one of a peptidomimetic macrocycle and an additional pharmaceutically-active agent, for example, any additional therapeutic agent described herein, is administered for from 2 to 21 consecutive days, followed by an optional drug holiday, followed by administration of the other of a peptidomimetic macrocycle and an additional pharmaceutically-active agent for from 2 to 21 consecutive days. In some embodiments, one of a peptidomimetic macrocycle and an additional pharmaceutically-active agent, for example, any additional therapeutic agent described herein, is administered for from 2 to 14 consecutive days, followed by a drug holiday of from 1 to 14 days, followed by administration of the other of a peptidomimetic macrocycle and an additional pharmaceutically-active agent for from 2 to 14 consecutive days. In some embodiments, one of a peptidomimetic macrocycle and an additional pharmaceutically-active agent, for example, any additional therapeutic agent described herein, is administered for from 3 to 7 consecutive days, followed by a drug holiday of from 3 to 10 days, followed by administration of the other of a peptidomimetic macrocycle and an additional pharmaceutically-active agent for from 3 to 7 consecutive days.

In some embodiments, a cyclin dependent kinase inhibitor will be administered first in the sequence, followed by an optional drug holiday, followed by administration of a peptidomimetic macrocycle. In some embodiments, a cyclin dependent kinase inhibitor is administered for from 3 to 21 consecutive days, followed by an optional drug holiday, followed by administration of a peptidomimetic macrocycle for from 3 to 21 consecutive days. In some embodiments, a cyclin dependent kinase inhibitor is administered for from 3 to 21 consecutive days, followed by a drug holiday of from 1 to 14 days, followed by administration of a peptidomimetic macrocycle for from 3 to 21 consecutive days. In some embodiments, a cyclin dependent kinase inhibitor is administered for from 3 to 21 consecutive days, followed by a drug holiday of from 3 to 14 days, followed by administration of a peptidomimetic macrocycle for from 3 to 21 consecutive days. In some embodiments, a cyclin dependent kinase inhibitor is administered for 21 consecutive days, followed by an optional drug holiday, followed by administration of a peptidomimetic macrocycle for 14 consecutive days. In some embodiments, a cyclin dependent kinase inhibitor is administered for 14 consecutive days, followed by a drug holiday of from 1 to 14 days, followed by administration of a peptidomimetic macrocycle for 14 consecutive days. In some embodiments, a cyclin dependent kinase inhibitor is administered for 7 consecutive days, followed by a drug holiday of from 3 to 10 days, followed by administration of a peptidomimetic macrocycle for 7 consecutive days. In some embodiments, a cyclin dependent kinase inhibitor is administered for 3 consecutive days, followed by a drug holiday of from 3 to 14 days, followed by administration of a peptidomimetic macrocycle for 7 consecutive days. In some embodiments, a cyclin dependent kinase inhibitor is administered for 3 consecutive days, followed by a drug holiday of from 3 to 10 days, followed by administration of a peptidomimetic macrocycle for 3 consecutive days.

In some embodiments, a peptidomimetic macrocycle will be administered first in the sequence, followed by an optional drug holiday, followed by administration of a cyclin dependent kinase inhibitor. In some embodiments, a peptidomimetic macrocycle is administered for from 3 to 21 consecutive days, followed by an optional drug holiday, followed by administration of a cyclin dependent kinase inhibitor for from 3 to 21 consecutive days. In some embodiments, a peptidomimetic macrocycle is administered for from 3 to 21 consecutive days, followed by a drug holiday of from 1 to 14 days, followed by administration of a cyclin dependent kinase inhibitor for from 3 to 21 consecutive days. In some embodiments, a peptidomimetic macrocycle is administered for from 3 to 21 consecutive days, followed by a drug holiday of from 3 to 14 days, followed by administration of a cyclin dependent kinase inhibitor for from 3 to 21 consecutive days. In some embodiments, a peptidomimetic macrocycle is administered for 21 consecutive days, followed by an optional drug holiday, followed by administration of a cyclin dependent kinase inhibitor for 14 consecutive days. In some embodiments, a peptidomimetic macrocycle s administered for 14 consecutive days, followed by a drug holiday of from 1 to 14 days, followed by administration of a cyclin dependent kinase inhibitor for 14 consecutive days. In some embodiments, a peptidomimetic macrocycle is administered for 7 consecutive days, followed by a drug holiday of from 3 to 10 days, followed by administration of a cyclin dependent kinase inhibitor for 7 consecutive days. In some embodiments, a peptidomimetic macrocycle is administered for 3 consecutive days, followed by a drug holiday of from 3 to 14 days, followed by administration of a cyclin dependent kinase inhibitor for 7 consecutive days. In some embodiments, a peptidomimetic macrocycle is administered for 3 consecutive days, followed by a drug holiday of from 3 to 10 days, followed by administration of a cyclin dependent kinase inhibitor for 3 consecutive days.

In some embodiments, a peptidomimetic macrocycle is administered once, twice, or thrice daily for 2, 3, 4, 5, 6, 7, 8, 9, 10, 11, 12, 13, 14, 15, 16, 17, 18, 19, 20, 21, 22, 23, 24, 25, 26, 27, 28, 29, or 30, consecutive days followed by 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days of rest (e.g., no administration of the peptidomimetic macrocycle/discontinuation of treatment) in a 1, 2, 3, 4, 5, 6, 7, 8, 9, 10, 11, 12, 13, 14, 15, 16, 17, 18, 19, 20, 21, 22, 23, 24, 25, 26, 27, or 28 day cycle; and the additional pharmaceutically-active agent, for example, any additional therapeutic agent described herein, is administered prior to, concomitantly with, or subsequent to administration of the peptidomimetic macrocycle on one or more days (e.g., on day 1 of cycle 1). In some embodiments, the combination therapy is administered for 1, 2, 3, 4, 5, 6, 7, 8, 9, 10, 12, or 13 cycles of 1, 2, 3, 4, 5, 6, 7, 8, 9, 10, 11, 12, 13, 14, 15, 16, 17, 18, 19, 20, 21, 22, 23, 24, 25, 26, 27, or 28 days. In some embodiments, the combination therapy is administered for 1 to 12 or 13 cycles of 28 days (e.g., about 12 months).

In some embodiments, provided herein is a method of treating a condition or disease comprising administering to a patient in need thereof a therapeutically effective amount of a peptidomimetic macrocycle in combination with a therapeutically effective amount of an additional pharmaceutically-active agent, for example, any additional therapeutic agent described herein, and a secondary active agent, such as a checkpoint inhibitor. In some embodiments, a peptidomimetic macrocycle is administered once, twice, or thrice daily for 2, 3, 4, 5, 6, 7, 8, 9, 10, 11, 12, 13, 14, 15, 16, 17, 18, 19, 20, 21, 22, 23, 24, 25, 26, 27, 28, 29, or 30, consecutive days followed by 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days of rest (e.g., no administration of the peptidomimetic macrocycle/discontinuation of treatment) in a 1, 2, 3, 4, 5, 6, 7, 8, 9, 10, 11, 12, 13, 14, 15, 16, 17, 18, 19, 20, 21, 22, 23, 24, 25, 26, 27, or 28 day cycle; the additional pharmaceutically-active agent, for example, any additional therapeutic agent described herein, is administered prior to, concomitantly with, or subsequent to administration of the peptidomimetic macrocycle on one or more days (e.g., on day 1 of cycle 1), and the secondary agent is administered daily, weekly, or monthly. In some embodiments, the combination therapy is administered for 1, 2, 3, 4, 5, 6, 7, 8, 9, 10, 12, or 13 cycles of 1, 2, 3, 4, 5, 6, 7, 8, 9, 10, 11, 12, 13, 14, 15, 16, 17, 18, 19, 20, 21, 22, 23, 24, 25, 26, 27, or 28 days. In some embodiments, the combination therapy is administered for 1 to 12 or 13 cycles of 28 days (e.g., about 12 months).

In some embodiments, administration of a combination therapy as described herein modulates expression levels of at least one checkpoint protein (e.g., PD-L1). Thus, provided herein are methods of determining the expression of at least of checkpoint proteins, where the determination of the expression level is performed before, during, and/or after administration of a combination therapy described herein. The checkpoint protein expression levels determined before, during, and/or after administration of a combination therapy as described herein can be compared against each other or standard controls. Such comparisons can translate into determination of the efficacy of the administered treatment where in one embodiment a level of decreased expression of a given checkpoint protein indicates a greater effectiveness of the combination therapy. In some embodiments, treatment using the combination therapies described herein can be monitored or determined using assays to determine expression levels of checkpoint proteins (e.g., PD-L1, TIM-3, LAG-3, CTLA-4, OX40, Treg, CD25, CD127, FoxP3). Determining the expression of such checkpoint proteins can be performed before, during, or after completion of treatment with a combination therapy described herein. Expression can be determined using techniques known in the art, including for example flow-cytometry.

In some embodiments, the components of the combination therapies described herein (e.g., a peptidomimetic macrocycle and a cyclin dependent kinase inhibitor) are cyclically administered to a patient. In some embodiments, a secondary active agent is co-administered in a cyclic administration with the combination therapies provided herein. Cycling therapy involves the administration of an active agent for a period of time, followed by a rest for a period of time, and repeating this sequential administration. Cycling therapy can be performed independently for each active agent (e.g., a peptidomimetic macrocycle and a cyclin dependent kinase inhibitor, and/or a secondary agent) over a prescribed duration of time. In some embodiments, the cyclic administration of each active agent is dependent upon one or more of the active agents administered to the subject. In some embodiments, administration of a peptidomimetic macrocycle or a cyclin dependent kinase inhibitor fixes the day(s) or duration of administration of each agent. In some embodiments, administration of a peptidomimetic macrocycle or a cyclin dependent kinase inhibitor fixes the days(s) or duration of administration of a secondary active agent.

In some embodiments, a peptidomimetic macrocycle, a cyclin dependent kinase inhibitor, and/or a secondary active agent is administered continually (e.g., daily, weekly, monthly) without a rest period. Cycling therapy can reduce the development of resistance to one or more of the therapies, avoid, or reduce the side effects of one of the therapies, and/or improve the efficacy of the treatment or therapeutic agent.

In some embodiments, the frequency of administration is in the range of about a daily dose to about a monthly dose. In some embodiments, administration is once a day, twice a day, three times a day, four times a day, once every other day, twice a week, once every week, once every two weeks, once every three weeks, or once every four weeks. In some embodiments, a compound for use in combination therapies described herein is administered once a day. In some embodiments, a compound for use in combination therapies described herein is administered twice a day. In some embodiments, a compound for use in combination therapies described herein is administered three times a day. In some embodiments, a compound for use in combination therapies described herein is administered four times a day.

In some embodiments, the frequency of administration of a peptidomimetic macrocycle is in the range of about a daily dose to about a monthly dose. In some embodiments, administration of a peptidomimetic macrocycle is once a day, twice a day, three times a day, four times a day, once every other day, twice a week, once every week, once every two weeks, once every three weeks, or once every four weeks. In some embodiments, a peptidomimetic macrocycle for use in combination therapies described herein is administered once a day. In some embodiments, a peptidomimetic macrocycle for use in combination therapies described herein is administered twice a day. In some embodiments, a peptidomimetic macrocycle for use in combination therapies described herein is administered three times a day. In some embodiments, a peptidomimetic macrocycle for use in combination therapies described herein is administered four times a day.

In some embodiments, the frequency of administration of an additional pharmaceutically-active agent, for example, any additional therapeutic agent described herein, is in the range of about a daily dose to about a monthly dose. In some embodiments, administration of an additional pharmaceutically-active agent, for example, any additional therapeutic agent described herein, is once a day, twice a day, three times a day, four times a day, once every other day, twice a week, once every week, once every two weeks, once every three weeks, or once every four weeks. In some embodiments, an additional pharmaceutically-active agent, for example, any additional therapeutic agent described herein, for use in combination therapies described herein is administered once a day. In some embodiments, an additional pharmaceutically-active agent, for example, any additional therapeutic agent described herein, for use in combination therapies described herein is administered twice a day. In some embodiments, an additional pharmaceutically-active agent, for example, any additional therapeutic agent described herein, for use in combination therapies described herein is administered three times a day. In some embodiments, an additional pharmaceutically-active agent, for example, any additional therapeutic agent described herein, for use in combination therapies described herein is administered four times a day.

In some embodiments, a compound for use in combination therapies described herein is administered once per day from one day to six months, from one week to three months, from one week to four weeks, from one week to three weeks, or from one week to two weeks. In some embodiments, a compound for use in combination therapies described herein is administered once per day for one week, two weeks, three weeks, or four weeks. In some embodiments, a compound for use in combination therapies described herein is administered once per day for one week. In some embodiments, a compound for use in combination therapies described herein is administered once per day for two weeks. In some embodiments, a compound for use in combination therapies described herein is administered once per day for three weeks. In some embodiments, a compound for use in combination therapies described herein is administered once per day for four weeks.

Therapeutic compositions may be administered 1, 2, 3, 4, 5, 6, 7, 8, 9, 10, 11, 12, 13, 14, 15, 16, 17, 18, 19, 20 or more times, and they may be administered every 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months.

In some embodiments, the periodic administration of a peptidomimetic macrocycle and/or the additional pharmaceutically-active agent, for example, any additional therapeutic agent described herein, is effected daily. In some embodiments, the periodic administration of a peptidomimetic macrocycle and/or the additional pharmaceutically-active agent, for example, any additional therapeutic agent described herein, is effected twice daily at one half the amount.

In some embodiments, the periodic administration of a peptidomimetic macrocycle and/or the additional pharmaceutically-active agent, for example, any additional therapeutic agent described herein, is effected once every 3 to 11 days; or once every 5 to 9 days; or once every 7 days; or once every 24 hours. In some embodiments, the periodic administration of a peptidomimetic macrocycle and/or the additional pharmaceutically-active agent, for example, any additional therapeutic agent described herein, is effected once every 2 days, 3 days, 4 days, 5 days, 6 days, 7 days, 8 days, 9 days, 10 days, 11 days, 12 days, 13 days, 14 days, 15 days, 6 days, 16 days, 17 days, 18 days, 19 days, 20 days, 21 days, 22 days, 23 days, 24 days, 25 days, 26 days, 27 days, 28 days, 29 days, or 30 days.

In some embodiments, the periodic administration of a peptidomimetic macrocycle and/or additional pharmaceutically-active agent is effected one, twice, or thrice daily.

For each administration schedule of a peptidomimetic macrocycle, the periodic administration of the additional pharmaceutically-active agent, for example, any additional therapeutic agent described herein, may be effected once every 16-32 hours; or once every 18-30 hours; or once every 20-28 hours; or once every 22-26 hours. In some embodiments, the administration of a peptidomimetic macrocycle substantially precedes the additional pharmaceutically-active agent, for example, any additional therapeutic agent described herein. In some embodiments, the administration of the additional pharmaceutically-active agent, for example, any additional therapeutic agent described herein, substantially precedes the administration of a peptidomimetic macrocycle.

In some embodiments, a peptidomimetic macrocycle and the additional pharmaceutically-active agent, for example, any additional therapeutic agent described herein, may be administered for a period of time of at least 4 days. In some embodiments, the period of time may be 5 days to 5 years; or 10 days to 3 years; or 2 weeks to 1 year; or 1 month to 6 months; or 3 months to 4 months. In some embodiments, a peptidomimetic macrocycle and the additional pharmaceutically-active agent, for example, any additional therapeutic agent described herein, may be administered for the lifetime of the subject.

### Pharmaceutical Compositions for Combination Treatment

According to certain embodiments, the peptidomimetic macrocycles and the additional pharmaceutically-active agent, for example, any additional therapeutic agent described herein, are administered within a single pharmaceutical composition. In some embodiments, the peptidomimetic macrocycles of the invention and the additional pharmaceutically-active agent, for example, any additional therapeutic agent described herein, can be provided in a single unit dosage form for being taken together. According to some embodiments, the pharmaceutical composition further comprises pharmaceutically-acceptable diluents or carrier. According to certain embodiments, the peptidomimetic macrocycles and the additional pharmaceutically-active agent, for example, any additional therapeutic agent described herein, are administered within different pharmaceutical composition. In some embodiments, the peptidomimetic macrocycles of the invention and the additional pharmaceutically-active agent, for example, any additional therapeutic agent described herein, can be provided in a single unit dosage as separate entities (e.g., in separate containers) to be administered simultaneously or with a certain time difference. In some embodiments, the peptidomimetic macrocycles of the disclosure and the additional pharmaceutically-active agent, for example, any additional therapeutic agent described herein, can be administered via the same route of administration. In some embodiments, the peptidomimetic macrocycles of the disclosure and the additional pharmaceutically-active agent, for example, any additional therapeutic agent described herein, can be administered via the different route of administration.

In some embodiments, the at least one additional pharmaceutical agent, for example, any additional therapeutic agent described herein, is administered at the therapeutic amount known to be used for treating the specific type of cancer. In some embodiments, the at least one additional pharmaceutical agent, for example, any additional therapeutic agent described herein, is administered in an amount lower than the therapeutic amount known to be used for treating the disease, i.e. a sub-therapeutic amount of the at least one additional pharmaceutical agent is administered.

A peptidomimetic macrocycle of the disclosure and at least one additional pharmaceutical agent, for example, any additional therapeutic agent described herein, administered to the subject can each be from about 0.01 mg/kg to about 100 mg/kg per body weight of the subject. In some embodiments, a peptidomimetic macrocycle of the disclosure and the at least one additional pharmaceutical agent, for example, any additional therapeutic agent described herein, administered to the subject can each be from about 0.01 mg/kg to about 1 mg/kg, 0.01 mg/kg to about 10 mg/kg, 0.01 mg/kg to about 100 mg/kg, 0.1 mg to about 1 mg/kg, 0.1 mg/kg to about 10 mg/kg, or 0.1 mg/kg to about 100 mg/kg per body weight of the subject. In some embodiments, the doses of a peptidomimetic macrocycle and additional therapeutic agent, for example, any additional therapeutic agent described herein, can be administered as a single dose or as multiple doses.

### Sequence Homology

Two or more peptides can share a degree of homology. A pair of peptides can have, for example, up to about 20% pairwise homology, up to about 25% pairwise homology, up to about 30% pairwise homology, up to about 35% pairwise homology, up to about 40% pairwise homology, up to about 45% pairwise homology, up to about 50% pairwise homology, up to about 55% pairwise homology, up to about 60% pairwise homology, up to about 65% pairwise homology, up to about 70% pairwise homology, up to about 75% pairwise homology, up to about 80% pairwise homology, up to about 85% pairwise homology, up to about 90% pairwise homology, up to about 95% pairwise homology, up to about 96% pairwise homology, up to about 97% pairwise homology, up to about 98% pairwise homology, up to about 99% pairwise homology, up to about 99.5% pairwise homology, or up to about 99.9% pairwise homology. A pair of peptides can have, for example, at least about 20% pairwise homology, at least about 25% pairwise homology, at least about 30% pairwise homology, at least about 35% pairwise homology, at least about 40% pairwise homology, at least about 45% pairwise homology, at least about 50% pairwise homology, at least about 55% pairwise homology, at least about 60% pairwise homology, at least about 65% pairwise homology, at least about 70% pairwise homology, at least about 75% pairwise homology, at least about 80% pairwise homology, at least about 85% pairwise homology, at least about 90% pairwise homology, at least about 95% pairwise homology, at least about 96% pairwise homology, at least about 97% pairwise homology, at least about 98% pairwise homology, at least about 99% pairwise homology, at least about 99.5% pairwise homology, at least about 99.9% pairwise homology.

Methods of Detecting Wild Type p53 and/or p53 Mutations

In some embodiments, a subject lacking p53-deactivating mutations is a candidate for cancer treatment with a compound of the invention. Cancer cells from patient groups should be assayed in order to determine p53-deactivating mutations and/or expression of wild type p53 prior to treatment with a compound of the invention.

The activity of the p53 pathway can be determined by the mutational status of genes involved in the p53 pathways, including, for example, AKT1, AKT2, AKT3, ALK, BRAF, CDK4, CDKN2A, DDR2, EGFR, ERBB2 (HER2), FGFR1, FGFR3, GNA11, GNQ, GNAS, KDR, KIT, KRAS, MAP2K1 (MEK1), MET, HRAS, NOTCH1, NRAS, NTRK2, PIK3CA, NF1, PTEN, RAC1, RB1, NTRK3, STK11, PIK3R1, TSC1, TSC2, RET, TP53, and VHL. Genes that modulate the activity of p53 can also be assessed, including, for example, kinases: ABL1, JAK1, JAAK2, JAK3; receptor tyrosine kinases: FLT3 and KIT; receptors: CSF3R, IL7R, MPL, and NOTCH1; transcription factors: BCOR, CEBPA, CREBBP, ETV6, GATA1, GATA2. MLL, KZF1, PAX5, RUNX1, STAT3, WT1, and TP53; epigenetic factors: ASXL1, DNMT3A, EZH2, KDM6A (UTX), SUZ12, TET2, PTPN11, SF3B1, SRSF2, U2AF35, ZRSR2; RAS proteins: HRAS, KRAS, and NRAS; adaptors CBL and CBL-B; FBXW7, IDH1, IDH2, and NPM1.

Cancer cell samples can be obtained, for example, from solid or liquid tumors via primary or metastatic tumor resection (e.g. pneumonectomy, lobetomy, wedge resection, and craniotomy) primary or metastatic disease biopsy (e.g. transbronchial or needle core), pleural or ascites fluid (e.g. FFPE cell pellet), bone marrow aspirate, bone marrow clot, and bone marrow biopsy, or macro-dissection of tumor rich areas (solid tumors).

To detect the p53 wild type gene and/or lack of p53 deactivation mutation in a tissue, cancerous tissue can be isolated from surrounding normal tissues. For example, the tissue can be isolated from paraffin or cryostat sections. Cancer cells can also be separated from normal cells by flow cytometry. If the cancer cells tissue is highly contaminated with normal cells, detection of mutations can be more difficult.

Various methods and assays for analyzing wild type p53 and/or p53 mutations are suitable for use in the invention. Non-limiting examples of assays include polymerase chain reaction (PCR), restriction fragment length polymorphism (RFLP), microarray, Southern Blot, Northern Blot, Western Blot, Eastern Blot, HandE staining, microscopic assessment of tumors, next-generation DNA sequencing (NGS) (e.g. extraction, purification, quantification, and amplification ofDNA, library preparation) immunohistochemistry, and fluorescent in situ hybridization (FISH).

A microarray allows a researcher to investigate multiple DNA sequences attached to a surface, for example, a DNA chip made of glass or silicon, or a polymeric bead or resin. The DNA sequences are hybridized with fluorescent or luminescent probes. The microarray can indicate the presence of oligonucleotide sequences in a sample based on hybridization of sample sequences to the probes, followed by washing and subsequent detection of the probes. Quantification of the fluorescent or luminescent signal indicates the presence of known oligonucleotide sequences in the sample.

PCR allows amplification of DNA oligomers rapidly, and can be used to identify an oligonucleotide sequence in a sample. PCR experiments involve contacting an oligonucleotide sample with a PCR mixture containing primers complementary to a target sequence, one or more DNA polymerase enzymes, deoxnucleotide triphosphate (dNTP) building blocks, including dATP, dGTP, dTTP, and dCTP, and suitable buffers, salts, and additives. If a sample contains an oligonucleotide sequence complementary to a pair of primers, the experiment amplifies the sample sequence, which can be collected and identified.

In some embodiments, an assay comprises amplifying a biomolecule from the cancer sample. The biomolecule can be a nucleic acid molecule, such as DNA or RNA. In some embodiments, the assay comprises circularization of a nucleic acid molecule, followed by digestion of the circularized nucleic acid molecule.

In some embodiments, the assay comprises contacting an organism, or a biochemical sample collected from an organism, such as a nucleic acid sample, with a library of oligonucleotides, such as PCR primers. The library can contain any number of oligonucleotide molecules. The oligonucleotide molecules can bind individual DNA or RNA motifs, or any combination of motifs described herein. The motifs can be any distance apart, and the distance can be known or unknown. In some embodiments, two or more oligonucleotides in the same library bind motifs a known distance apart in a parent nucleic acid sequence. Binding of the primers to the parent sequence can take place based on the complementarity of the primers to the parent sequence. Binding can take place, for example, under annealing, or under stringent conditions.

In some embodiments, the results of an assay are used to design a new oligonucleotide sequence for future use. In some embodiments, the results of an assay are used to design a new oligonucleotide library for future use. In some embodiments, the results of an assay are used to revise, refine, or update an existing oligonucleotide library for future use. For example, an assay can reveal that a previously-undocumented nucleic acid sequence is associated with the presence of a target material. This information can be used to design or redesign nucleic acid molecules and libraries.

In some embodiments, one or more nucleic acid molecules in a library comprise a barcode tag. In some embodiments, one or more of the nucleic acid molecules in a library comprise type I or type II restriction sites suitable for circularization and cutting an amplified sample nucleic acid sequence. Such primers can be used to circularize a PCR product and cut the PCR product to provide a product nucleic acid sequence with a sequence that is organized differently from the nucleic acid sequence native to the sample organism.

After a PCR experiment, the presence of an amplified sequence can be verified. Non-limiting examples of methods for finding an amplified sequence include DNA sequencing, whole transcriptome shotgun sequencing (WTSS, or RNA-seq), mass spectrometry (MS), microarray, pyrosequencing, column purification analysis, polyacrylamide gel electrophoresis, and index tag sequencing of a PCR product generated from an index-tagged primer.

In some embodiments, more than one nucleic acid sequence in the sample organism is amplified. Non-limiting examples of methods of separating different nucleic acid sequences in a PCR product mixture include column purification, high performance liquid chromatography (HPLC), HPLC/MS, polyacrylamide gel electrophoresis, size exclusion chromatography.

The amplified nucleic acid molecules can be identified by sequencing. Nucleic acid sequencing can be done on automated instrumentation. Sequencing experiments can be done in parallel to analyze tens, hundreds, or thousands of sequences simultaneously. Non-limiting examples of sequencing techniques follow.

In pyrosequencing, DNA is amplified within a water droplet containing a single DNA template bound to a primer-coated bead in an oil solution. Nucleotides are added to a growing sequence, and the addition of each base is evidenced by visual light.

Ion semiconductor sequencing detects the addition of a nucleic acid residue as an electrical signal associated with a hydrogen ion liberated during synthesis. A reaction well containing a template is flooded with the four types of nucleotide building blocks, one at a time. The timing of the electrical signal identifies which building block was added, and identifies the corresponding residue in the template.

DNA nanoball uses rolling circle replication to amplify DNA into nanoballs. Unchained sequencing by ligation of the nanoballs reveals the DNA sequence.

In a reversible dyes approach, nucleic acid molecules are annealed to primers on a slide and amplified. Four types of fluorescent dye residues, each complementary to a native nucleobase, are added, the residue complementary to the next base in the nucleic acid sequence is added, and unincorporated dyes are rinsed from the slide. Four types of reversible terminator bases (RT-bases) are added, and non-incorporated nucleotides are washed away. Fluorescence indicates the addition of a dye residue, thus identifying the complementary base in the template sequence. The dye residue is chemically removed, and the cycle repeats.

Detection of point mutations can be accomplished by molecular cloning of the p53 allele(s) present in the cancer cell tissue and sequencing that allele(s). Alternatively, the polymerase chain reaction can be used to amplify p53 gene sequences directly from a genomic DNA preparation from the cancer cell tissue. The DNA sequence of the amplified sequences can then be determined. Specific deletions of p53 genes can also be detected. For example, restriction fragment length polymorphism (RFLP) probes for the p53 gene or surrounding marker genes can be used to score loss of a p53 allele.

Loss of wild type p53 genes can also be detected on the basis of the loss of a wild type expression product of the p53 gene. Such expression products include both the mRNA as well as the p53 protein product itself. Point mutations can be detected by sequencing the mRNA directly or via molecular cloning of cDNA made from the mRNA. The sequence of the cloned cDNA can be determined using DNA sequencing techniques. The cDNA can also be sequenced via the polymerase chain reaction (PCR).

Alternatively, mismatch detection can be used to detect point mutations in the p53 gene or the mRNA product. The method can involve the use of a labeled riboprobe that is complementary to the human wild type p53 gene. The riboprobe and either mRNA or DNA isolated from the cancer cell tissue are annealed (hybridized) together and subsequently digested with the enzyme RNase A which is able to detect some mismatches in a duplex RNA structure. If a mismatch is detected by RNase A, the enzyme cleaves at the site of the mismatch. Thus, when the annealed RNA preparation is separated on an electrophoretic gel matrix, if a mismatch has been detected and cleaved by RNase A, an RNA product is seen that is smaller than the full-length duplex RNA for the riboprobe and the p53 mRNA or DNA. The riboprobe need not be the full length of the p53 mRNA or gene but can be a segment of either. If the riboprobe comprises only a segment of the p53 mRNA or gene it will be desirable to use a number of these probes to screen the whole mRNA sequence for mismatches.

In similar fashion, DNA probes can be used to detect mismatches, through enzymatic or chemical cleavage. Alternatively, mismatches can be detected by shifts in the electrophoretic mobility of mismatched duplexes relative to matched duplexes. With either riboprobes or DNA probes, the cellular mRNA or DNA which might contain a mutation can be amplified using PCR before hybridization.

DNA sequences of the p53 gene from the cancer cell tissue which have been amplified by use of polymerase chain reaction can also be screened using allele-specific probes. These probes are nucleic acid oligomers, each of which contains a region of the p53 gene sequence harboring a known mutation. For example, one oligomer can be about 30 nucleotides in length, corresponding to a portion of the p53 gene sequence. At the position coding for the 175th codon of p53 gene the oligomer encodes an alanine, rather than the wild type codon valine. By use of a battery of such allele-specific probes, the PCR amplification products can be screened to identify the presence of a previously identified mutation in the p53 gene. Hybridization of allele-specific probes with amplified p53 sequences can be performed, for example, on a nylon filter. Hybridization to a particular probe indicates the presence of the same mutation in the cancer cell tissue as in the allele-specific probe.

The identification of p53 gene structural changes in cancer cells can be facilitated through the application of a diverse series of high resolution, high throughput microarray platforms. Essentially two types of array include those that carry PCR products from cloned nucleic acids (e.g. cDNA, BACs, cosmids) and those that use oligonucleotides. The methods can provide a way to survey genome wide DNA copy number abnormalities and expression levels to allow correlations between losses, gains and amplifications in cancer cells with genes that are over- and under-expressed in the same samples. The gene expression arrays that provide estimates of mRNA levels in cancer cells have given rise to exon-specific arrays that can identify both gene expression levels, alternative splicing events and mRNA processing alterations.

Oligonucleotide arrays can be used to interrogate single nucleotide polymorphisms (SNPs) throughout the genome for linkage and association studies and these have been adapted to quantify copy number abnormalities and loss of heterozygosity events. DNA sequencing arrays can allow resequencing of chromosome regions, exomes, and whole genomes.

SNP-based arrays or other gene arrays or chips can determine the presence of wild type p53 allele and the structure of mutations. A single nucleotide polymorphism (SNP), a variation at a single site in DNA, is the most frequent type of variation in the genome. For example, there are an estimated 5-10 million SNPs in the human genome. SNPs can be synonymous or nonsynonymous substitutions. Synonymous SNP substitutions do not result in a change of amino acid in the protein due to the degeneracy of the genetic code, but can affect function in other ways. For example, a seemingly silent mutation in a gene that codes for a membrane transport protein can slow down translation, allowing the peptide chain to misfold, and produce a less functional mutant membrane transport protein. Nonsynonymous SNP substitutions can be missense substitutions or nonsense substitutions. Missense substitutions occur when a single base change results in change in amino acid sequence of the protein and malfunction thereof leads to disease. Nonsense substitutions occur when a point mutation results in a premature stop codon, or a nonsense codon in the transcribed mRNA, which results in a truncated and usually, nonfunctional, protein product. As SNPs are highly conserved throughout evolution and within a population, the map of SNPs serves as an excellent genotypic marker for research. SNP array is a useful tool to study the whole genome.

In addition, SNP array can be used for studying the Loss Of Heterozygosity (LOH). LOH is a form of allelic imbalance that can result from the complete loss of an allele or from an increase in copy number of one allele relative to the other. While other chip-based methods (e.g., comparative genomic hybridization can detect only genomic gains or deletions), SNP array has the additional advantage of detecting copy number neutral LOH due to uniparental disomy (UPD). In UPD, one allele or whole chromosome from one parent are missing leading to reduplication of the other parental allele (uni-parental=from one parent, disomy=duplicated). In a disease setting this occurrence can be pathologic when the wild type allele (e.g., from the mother) is missing and instead two copies of the heterozygous allele (e.g., from the father) are present. This usage of SNP array has a huge potential in cancer diagnostics as LOH is a prominent characteristic of most human cancers. SNP array technology have shown that cancers (e.g. gastric cancer, liver cancer, etc.) and hematologic malignancies (ALL, MDS, CML etc) have a high rate of LOH due to genomic deletions or UPD and genomic gains. In the present disclosure, using high density SNP array to detect LOH allows identification of pattern of allelic imbalance to determine the presence of wild type p53 allele.

Mutations of wild type p53 genes can also be detected on the basis of the mutation of a wild type expression product of the p53 gene. Such expression products include both the mRNA as well as the p53 protein product itself. Point mutations can be detected by sequencing the mRNA directly or via molecular cloning of cDNA made from the mRNA. The sequence of the cloned cDNA can be determined using DNA sequencing techniques. The cDNA can also be sequenced via the polymerase chain reaction (PCR). A panel of monoclonal antibodies could be used in which each of the epitopes involved in p53 functions are represented by a monoclonal antibody. Loss or perturbation of binding of a monoclonal antibody in the panel can indicate mutational alteration of the p53 protein and thus of the p53 gene itself. Mutant p53 genes or gene products can also be detected in body samples, including, for example, serum, stool, urine, and sputum. The same techniques discussed above for detection of mutant p53 genes or gene products in tissues can be applied to other body samples.

Loss of wild type p53 genes can also be detected by screening for loss of wild type p53 protein function. Although all of the functions which the p53 protein undoubtedly possesses have yet to be elucidated, at least two specific functions are known. Protein p53 binds to the SV40 large T antigen as well as to the adenovirus E1B antigen. Loss of the ability of the p53 protein to bind to either or both of these antigens indicates a mutational alteration in the protein which reflects a mutational alteration of the gene itself. Alternatively, a panel of monoclonal antibodies could be used in which each of the epitopes involved in p53 functions are represented by a monoclonal antibody. Loss or perturbation of binding of a monoclonal antibody in the panel would indicate mutational alteration of the p53 protein and thus of the p53 gene itself. Any method for detecting an altered p53 protein can be used to detect loss of wild type p53 genes.

### Assays

The properties of peptidomimetic macrocycles are assayed, for example, by using the methods described below. In some embodiments, a peptidomimetic macrocycle has improved biological properties relative to a corresponding polypeptide lacking the substituents described herein.

a. Assays to Determine α-Helicity

In solution, the secondary structure of polypeptides with α-helical domains will reach a dynamic equilibrium between random coil structures and α-helical structures, often expressed as a “percent helicity”. Thus, for example, alpha-helical domains are predominantly random coils in solution, with α-helical content usually under 25%. Peptidomimetic macrocycles with optimized linkers, on the other hand, possess, for example, an alpha-helicity that is at least two-fold greater than that of a corresponding uncrosslinked polypeptide. In some embodiments, macrocycles will possess an alpha-helicity of greater than 50%. To assay the helicity of peptidomimetic macrocycles, the compounds are dissolved in an aqueous solution (e.g. 50 mM potassium phosphate solution at pH 7, or distilled H₂O, to concentrations of 25-50 μM). Circular dichroism (CD) spectra are obtained on a spectropolarimeter using standard measurement parameters (e.g. temperature, 20° C.; wavelength, 190-260 nm; step resolution, 0.5 nm; speed, 20 nm/sec; accumulations, 10; response, 1 sec; bandwidth, 1 nm; path length, 0.1 cm). The α-helical content of each peptide is calculated by dividing the mean residue ellipticity (e.g. [Φ]222obs) by the reported value for a model helical decapeptide.

b. Assay to Determine Melting Temperature (Tₘ)

A peptidomimetic macrocycle comprising a secondary structure such as an α-helix exhibits, for example, a higher melting temperature than a corresponding uncrosslinked polypeptide. Peptidomimetic macrocycles exhibit Tₘ of >60° C. representing a highly stable structure in aqueous solutions. To assay the effect of macrocycle formation on melting temperature, peptidomimetic macrocycles or unmodified peptides are dissolved in distilled H₂O (e.g. at a final concentration of 50 μM) and the Tₘ is determined by measuring the change in ellipticity over a temperature range (e.g. 4 to 95° C.) on a spectropolarimeter using standard parameters (e.g. wavelength 222 nm; step resolution, 0.5 nm; speed, 20 nm/sec; accumulations, 10; response, 1 sec; bandwidth, 1 nm; temperature increase rate: 1° C./min; path length, 0.1 cm).

c. Protease Resistance Assay

The amide bond of the peptide backbone is susceptible to hydrolysis by proteases, thereby rendering peptidic compounds vulnerable to rapid degradation in vivo. Peptide helix formation, however, buries the amide backbone and therefore can shield it from proteolytic cleavage. The peptidomimetic macrocycles can be subjected to in vitro trypsin proteolysis to assess for any change in degradation rate compared to a corresponding uncrosslinked polypeptide. For example, the peptidomimetic macrocycle and a corresponding uncrosslinked polypeptide are incubated with trypsin agarose and the reactions quenched at various time points by centrifugation and subsequent HPLC injection to quantitate the residual substrate by ultraviolet absorption at 280 nm. Briefly, the peptidomimetic macrocycle and peptidomimetic precursor (5 mcg) are incubated with trypsin agarose (S/E ˜125) for 0, 10, 20, 90, and 180 minutes. Reactions are quenched by tabletop centrifugation at high speed; remaining substrate in the isolated supernatant is quantified by HPLC-based peak detection at 280 nm. The proteolytic reaction displays first order kinetics and the rate constant, k, is determined from a plot of ln [S] versus time (k=−1×slope).

d. Ex Vivo Stability Assay

Peptidomimetic macrocycles with optimized linkers possess, for example, an ex vivo half-life that is at least two-fold greater than that of a corresponding uncrosslinked polypeptide, and possess an ex vivo half-life of 12 hours or more. For ex vivo serum stability studies, a variety of assays can be used. For example, a peptidomimetic macrocycle and a corresponding uncrosslinked polypeptide (2 mcg) are incubated with fresh mouse, rat and/or human serum (2 mL) at 37° C. for 0, 1, 2, 4, 8, and 24 hours. To determine the level of intact compound, the following procedure can be used: The samples are extracted by transferring 100 μL of sera to 2 ml centrifuge tubes followed by the addition of 10 μL of 50% formic acid and 500 μL acetonitrile and centrifugation at 14,000 RPM for 10 min at 4±2° C. The supernatants are then transferred to fresh 2 ml tubes and evaporated on Turbovap under N₂<10 psi, 37° C. The samples are reconstituted in 100 μL of 50:50 acetonitrile:water and submitted to LC-MS/MS analysis.

e. In Vitro Binding Assays

To assess the binding and affinity of peptidomimetic macrocycles and peptidomimetic precursors to acceptor proteins, a fluorescence polarization assay (FPA) is used, for example. The FPA technique measures the molecular orientation and mobility using polarized light and fluorescent tracer. When excited with polarized light, fluorescent tracers (e.g., FITC) attached to molecules with high apparent molecular weights (e.g. FITC-labeled peptides bound to a large protein) emit higher levels of polarized fluorescence due to their slower rates of rotation as compared to fluorescent tracers attached to smaller molecules (e.g. FITC-labeled peptides that are free in solution).

For example, fluoresceinated peptidomimetic macrocycles (25 nM) are incubated with the acceptor protein (25-1000 nM) in binding buffer (140 mM NaCl, 50 mM Tris-HCL, pH 7.4) for 30 minutes at room temperature. Binding activity is measured, for example, by fluorescence polarization on a luminescence spectrophotometer. Kd values can be determined by nonlinear regression analysis using, for example, GraphPad Prism software. A peptidomimetic macrocycle shows, In some embodiments, similar or lower Kd than a corresponding uncrosslinked polypeptide.

f. In Vitro Displacement Assays to Characterize Antagonists of Peptide-Protein Interactions

To assess the binding and affinity of compounds that antagonize the interaction between a peptide and an acceptor protein, a fluorescence polarization assay (FPA) utilizing a fluoresceinated peptidomimetic macrocycle derived from a peptidomimetic precursor sequence is used, for example. The FPA technique measures the molecular orientation and mobility using polarized light and fluorescent tracer. When excited with polarized light, fluorescent tracers (e.g., FITC) attached to molecules with high apparent molecular weights (e.g. FITC-labeled peptides bound to a large protein) emit higher levels of polarized fluorescence due to their slower rates of rotation as compared to fluorescent tracers attached to smaller molecules (e.g. FITC-labeled peptides that are free in solution). A compound that antagonizes the interaction between the fluoresceinated peptidomimetic macrocycle and an acceptor protein will be detected in a competitive binding FPA experiment.

For example, putative antagonist compounds (1 nM to 1 mM) and a fluoresceinated peptidomimetic macrocycle (25 nM) are incubated with the acceptor protein (50 nM) in binding buffer (140 mM NaCl, 50 mM Tris-HCL, pH 7.4) for 30 minutes at room temperature. Antagonist binding activity is measured, for example, by fluorescence polarization on a luminescence spectrophotometer. Kd values can be determined by nonlinear regression analysis. Any class of molecule, such as small organic molecules, peptides, oligonucleotides or proteins can be examined as putative antagonists in this assay.

g. Assay for Protein-Ligand Binding by Affinity Selection-Mass Spectrometry

To assess the binding and affinity of test compounds for proteins, an affinity-selection mass spectrometry assay is used, for example. Protein-ligand binding experiments are conducted according to the following representative procedure outlined for a system-wide control experiment using 1 μM peptidomimetic macrocycle plus 5 μM hMDM2. A 1 μL DMSO aliquot of a 40 μM stock solution of peptidomimetic macrocycle is dissolved in 19 μL of PBS (50 mM, pH 7.5 Phosphate buffer containing 150 mM NaCl). The resulting solution is mixed by repeated pipetting and clarified by centrifugation at 10 000 g for 10 min. To a 4 μL aliquot of the resulting supernatant is added 4 μL of 10 μM hMDM2 in PBS. Each 8.0 μL experimental sample thus contains 40 pmol (1.5 μg) of protein at 5.0 μM concentration in PBS plus 1 μM peptidomimetic macrocycle and 2.5% DMSO. Duplicate samples thus prepared for each concentration point are incubated for 60 min at room temperature, and then chilled to 4° C. prior to size-exclusion chromatography-LC-MS analysis of 5.0 μL injections. Samples containing a target protein, protein-ligand complexes, and unbound compounds are injected onto an SEC column, where the complexes are separated from non-binding component by a rapid SEC step. The SEC column eluate is monitored using UV detectors to confirm that the early-eluting protein fraction, which elutes in the void volume of the SEC column, is well resolved from unbound components that are retained on the column. After the peak containing the protein and protein-ligand complexes elutes from the primary UV detector, it enters a sample loop where it is excised from the flow stream of the SEC stage and transferred directly to the LC-MS via a valving mechanism. The (M+3H)³⁺ ion of the peptidomimetic macrocycle is observed by ESI-MS at the expected m/z, confirming the detection of the protein-ligand complex.

h. Assay for Protein-Ligand K_{d} Titration Experiments

To assess the binding and affinity of test compounds for proteins, a protein-ligand Kd titration experiment is performed, for example. Protein-ligand K_{d} titrations experiments are conducted as follows: 2 μL DMSO aliquots of a serially diluted stock solution of titrant peptidomimetic macrocycle (5, 2.5, . . . , 0.098 mM) are prepared then dissolved in 38 μL of PBS. The resulting solutions are mixed by repeated pipetting and clarified by centrifugation at 10 000 g for 10 min. To 4.0 μL aliquots of the resulting supernatants is added 4.0 μL of 10 μM hMDM2 in PBS. Each 8.0 μL experimental sample thus contains 40 pmol (1.5 μg) of protein at 5.0 μM concentration in PBS, varying concentrations (125, 62.5, . . . , 0.24 μM) of the titrant peptide, and 2.5% DMSO. Duplicate samples thus prepared for each concentration point are incubated at room temperature for 30 min, then chilled to 4° C. prior to SEC-LC-MS analysis of 2.0 μL injections. The (M+H)¹⁺, (M+2H)²⁺, (M+3H)³⁺, and/or (M+Na)¹⁺ ion is observed by ESI-MS; extracted ion chromatograms are quantified, then fit to equations to derive the binding affinity K_{d}.

i. Assay for Competitive Binding Experiments by Affinity Selection-Mass Spectrometry

To determine the ability of test compounds to bind competitively to proteins, an affinity selection mass spectrometry assay is performed, for example. A mixture of ligands at 40 μM per component is prepared by combining 2 μL aliquots of 400 μM stocks of each of the three compounds with 14 μL of DMSO. Then, 1 μL aliquots of this 40 μM per component mixture are combined with 1 μL DMSO aliquots of a serially diluted stock solution of titrant peptidomimetic macrocycle (10, 5, 2.5, . . . , 0.078 mM). These 2 μL samples are dissolved in 38 μL of PBS. The resulting solutions were mixed by repeated pipetting and clarified by centrifugation at 10 000 g for 10 min. To 4.0 μL aliquots of the resulting supernatants is added 4.0 μL of 10 μM hMDM2 protein in PBS. Each 8.0 μL experimental sample thus contains 40 pmol (1.5 μg) of protein at 5.0 μM concentration in PBS plus 0.5 μM ligand, 2.5% DMSO, and varying concentrations (125, 62.5, . . . , 0.98 μM) of the titrant peptidomimetic macrocycle. Duplicate samples thus prepared for each concentration point are incubated at room temperature for 60 min, then chilled to 4° C. prior to SEC-LC-MS analysis of 2.0 μL injections.

j. Binding Assays in Intact Cells

It is possible to measure binding of peptides or peptidomimetic macrocycles to their natural acceptors in intact cells by immunoprecipitation experiments. For example, intact cells are incubated with fluoresceinated (FITC-labeled) compounds for 4 hrs in the absence of serum, followed by serum replacement and further incubation that ranges from 4-18 hrs. Cells are then pelleted and incubated in lysis buffer (50 mM Tris [pH 7.6], 150 mM NaCl, 1% CHAPS and protease inhibitor cocktail) for 10 minutes at 4° C. Extracts are centrifuged at 14,000 rpm for 15 minutes and supernatants collected and incubated with 10 μL goat anti-FITC antibody for 2 hrs, rotating at 4° C. followed by further 2 hrs incubation at 4° C. with protein A/G Sepharose (50 μL of 50% bead slurry). After quick centrifugation, the pellets are washed in lysis buffer containing increasing salt concentration (e.g., 150, 300, 500 mM). The beads are then re-equilibrated at 150 mM NaCl before addition of SDS-containing sample buffer and boiling. After centrifugation, the supernatants are optionally electrophoresed using 4%-12% gradient Bis-Tris gels followed by transfer into Immobilon-P membranes. After blocking, blots are optionally incubated with an antibody that detects FITC and also with one or more antibodies that detect proteins that bind to the peptidomimetic macrocycle.

k. Cellular Penetrability Assays

A peptidomimetic macrocycle is, for example, more cell penetrable compared to a corresponding uncrosslinked macrocycle. Peptidomimetic macrocycles with optimized linkers possess, for example, cell penetrability that is at least two-fold greater than a corresponding uncrosslinked macrocycle, and often 20% or more of the applied peptidomimetic macrocycle will be observed to have penetrated the cell after 4 hours. To measure the cell penetrability of peptidomimetic macrocycles and corresponding uncrosslinked macrocycle, intact cells are incubated with fluorescently-labeled (e.g. fluoresceinated) peptidomimetic macrocycles or corresponding uncrosslinked macrocycle (10 μM) for 4 hrs in serum free media at 37° C., washed twice with media and incubated with trypsin (0.25%) for 10 min at 37° C. The cells are washed again and resuspended in PBS. Cellular fluorescence is analyzed.

l. Cellular Efficacy Assays

The efficacy of certain peptidomimetic macrocycles is determined, for example, in cell-based killing assays using a variety of tumorigenic and non-tumorigenic cell lines and primary cells derived from human or mouse cell populations. Cell viability is monitored, for example, over 24-96 hrs of incubation with peptidomimetic macrocycles (0.5 to 50 μM) to identify those that kill at EC₅₀<10 μM. Several standard assays that measure cell viability are commercially available and are optionally used to assess the efficacy of the peptidomimetic macrocycles. In addition, assays that measure Annexin V and caspase activation are optionally used to assess whether the peptidomimetic macrocycles kill cells by activating the apoptotic machinery. For example, the Cell Titer-glo assay is used which determines cell viability as a function of intracellular ATP concentration.

m. In Vivo Stability Assay

To investigate the in vivo stability of the peptidomimetic macrocycles, the compounds are, for example, administered to mice and/or rats by IV, IP, PO or inhalation routes at concentrations ranging from 0.1 to 50 mg/kg and blood specimens withdrawn at 0′, 5′, 15′, 30′, 1 hr, 4 hrs, 8 hrs and 24 hours post-injection. Levels of intact compound in 25 μL of fresh serum are then measured by LC-MS/MS as above.

n. In Vivo Efficacy in Animal Models

To determine the anti-oncogenic activity of peptidomimetic macrocycles in vivo, the compounds are, for example, given alone (IP, IV, PO, by inhalation or nasal routes) or in combination with sub-optimal doses of relevant chemotherapy (e.g., cyclophosphamide, doxorubicin, etoposide). In one example, 5×10⁶ RS4;11 cells (established from the bone marrow of a patient with acute lymphoblastic leukemia) that stably express luciferase are injected by tail vein in NOD-SCID mice 3 hrs after they have been subjected to total body irradiation. If left untreated, this form of leukemia is fatal in 3 weeks in this model. The leukemia is readily monitored, for example, by injecting the mice with D-luciferin (60 mg/kg) and imaging the anesthetized animals. Total body bioluminescence is quantified by integration of photonic flux (photons/sec) by Living Image Software. Peptidomimetic macrocycles alone or in combination with sub-optimal doses of relevant chemotherapeutics agents are, for example, administered to leukemic mice (10 days after injection/day 1 of experiment, in bioluminescence range of 14-16) by tail vein or IP routes at doses ranging from 0.1 mg/kg to 50 mg/kg for 7 to 21 days. Optionally, the mice are imaged throughout the experiment every other day and survival monitored daily for the duration of the experiment. Expired mice are optionally subjected to necropsy at the end of the experiment. Another animal model is implantation into NOD-SCID mice of DoHH2, a cell line derived from human follicular lymphoma that stably expresses luciferase. These in vivo tests optionally generate preliminary pharmacokinetic, pharmacodynamic and toxicology data.

o. Clinical Trials

To determine the suitability of the peptidomimetic macrocycles for treatment of humans, clinical trials are performed. For example, patients diagnosed with cancer and in need of treatment can be selected and separated in treatment and one or more control groups, wherein the treatment group is administered a peptidomimetic macrocycle, while the control groups receive a placebo or a known anti-cancer drug. The treatment safety and efficacy of the peptidomimetic macrocycles can thus be evaluated by performing comparisons of the patient groups with respect to factors such as survival and quality-of-life. In this example, the patient group treated with a peptidomimetic macrocycle can show improved long-term survival compared to a patient control group treated with a placebo.

### EXAMPLES

### Example 1: Synthesis of 6-chlorotryptophan Fmoc Amino Acids

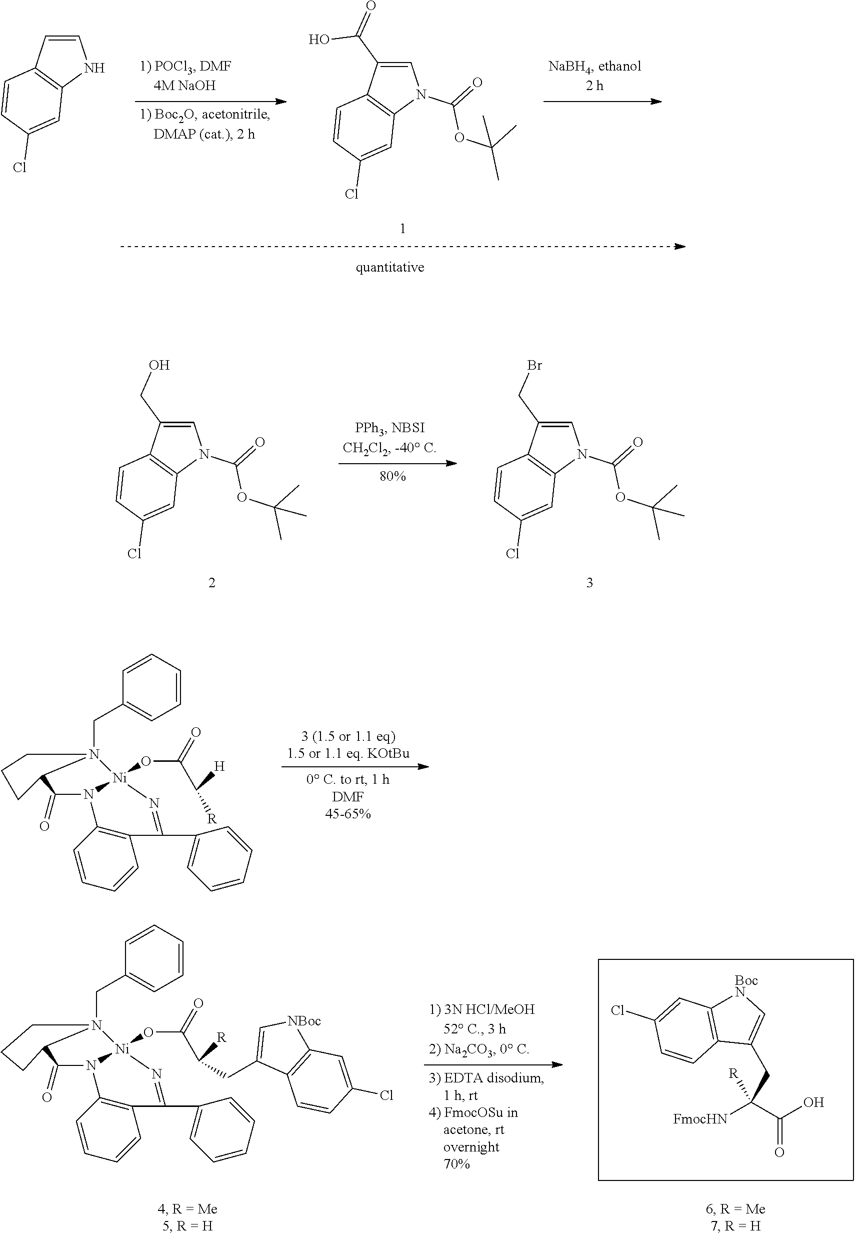

### Tert-butyl 6-chloro-3-formyl-1H-indole-1-carboxylate, 1

To a stirred solution of dry DMF (12 mL) was added dropwise POCl₃ (3.92 mL, 43 mmol, 1.3 equiv) at 0° C. under argon. The solution was stirred at 0° C. for 20 min before a solution of 6-chloroindole (5.0 g, 33 mmol, 1 eq.) in dry DMF (30 mL) was added dropwise. The resulting mixture was warmed to room temperature and stirred for an additional 2.5 h. Water (50 mL) was added to the reaction mixture, and the solution was neutralized with 4M aqueous NaOH (pH ˜8). The resulting solid was filtered off, washed with water, and dried under vacuum. This material was used in the next step without additional purification.

To a stirred solution of the crude formyl indole (33 mmol, 1 eq.) in THF (150 mL) was added successively Boc₂O (7.91 g, 36.3 mmol, 1.1 equiv) and DMAP (0.4 g, 3.3 mmol, 0.1 equiv) at room temperature under N₂. The resulting mixture was stirred at room temperature for 1.5 h, and the solvent was evaporated under reduced pressure. The residue was taken up in EtOAc and washed with 1N HCl, dried, and concentrated to afford formyl indole 1 (9 g, 98% over 2 steps) as a white solid. ¹H NMR (CDCl₃) δ: 1.70 (s, Boc, 9H); 7.35 (dd, 1H); 8.21 (m, 3H); 10.07 (s, 1H).

### Tert-butyl 6-chloro-3-(hydroxymethyl)-1H-indole-1-carboxylate, 2

To a solution of compound 1 (8.86 g, 32 mmol, 1 eq.) in ethanol (150 mL) was added NaBH₄ (2.4 g, 63 mmol, 2 eq.). The reaction was stirred for 3 h at room temperature. The reaction mixture was concentrated, and the residue was poured into diethyl ether and water. The organic layer was separated, dried over magnesium sulfate, and concentrated to give a white solid (8.7 g, 98%). This material was directly used in the next step without additional purification. ¹H NMR (CDCl₃) δ: 1.65 (s, Boc, 9H); 4.80 (s, 2H, CH₂); 7.21 (dd, 1H); 7.53 (m, 2H); 8.16 (bs, 1H).

### Tert-butyl 3-(bromomethyl)-6-chloro-1H-indole-1-carboxylate, 3

To a solution of compound 2 (4.1 g, 14.6 mmol, 1 eq.) in dichloromethane (50 mL) under argon was added a solution of triphenylphosphine (4.59 g, 17.5 mmol, 1.2 eq.) in dichloromethane (50 mL) at −40° C. The reaction was stirred for 30 min at 40° C. NBS (3.38 g, 19 mmol, 1.3 eq.) was then added to the reaction mixture. The resulting mixture was warmed to room temperature and stirred overnight. Dichloromethane was evaporated, carbon tetrachloride (100 mL) was added, and the mixture was stirred for 1 h and filtrated. The filtrate was concentrated, loaded on a silica plug, and quickly eluted with 25% EtOAc in hexanes. The solution was concentrated to afford a white foam (3.84 g, 77%). ¹H NMR (CDCl₃) δ: 1.66 (s, Boc, 9H); 4.63 (s, 2H, CH₂); 7.28 (dd, 1H); 7.57 (d, 1H); 7.64 (bs, 1H); 8.18 (bs, 1H).

### αMe-6Cl-Trp(Boc)-Ni—S-BPB, 4

To S-Ala-Ni—S-BPB (2.66 g, 5.2 mmol, 1 eq.) and KO-tBu (0.87 g, 7.8 mmol, 1.5 eq.) was added 50 mL of DMF under argon. The bromide derivative compound 3 (2.68 g, 7.8 mmol, 1.5 eq.) was dissolved in DMF (5.0 mL) and added to the reaction mixture using a syringe. The reaction mixture was stirred at ambient temperature for 1 h. The solution was then quenched with 5% aqueous acetic acid and diluted with water. The desired product was extracted in dichloromethane, dried, and concentrated. The oily product 4 was purified by flash chromatography (solid loading) on normal phase using EtOAc and hexanes as eluents to give a red solid (1.78 g, 45% yield). M+H calc. 775.21, M+H obs. 775.26; ¹H NMR (CDCl₃) δ: 1.23 (s, 3H, cMe); 1.56 (m, 11H, Boc+CH₂); 1.82-2.20 (m, 4H, 2CH₂); 3.03 (m, 1H, CH_{α}); 3.24 (m, 2H, CH₂); 3.57 and 4.29 (AB system, 2H, CH₂ (benzyl), J=12.8 Hz); 6.62 (d, 2H); 6.98 (d, 1H); 7.14 (m, 2H); 7.23 (m, 1H); 7.32-7.36 (m, 5H); 7.50 (m, 2H); 7.67 (bs, 1H); 7.98 (d, 2H); 8.27 (m, 2H).

### 6Cl-Trp(Boc)-Ni—S-BPB, 5

To Gly-Ni—S-BPB (4.6 g, 9.2 mmol, 1 eq.) and KO-tBu (1.14 g, 10.1 mmol, 1.1 eq.) was added 95 mL of DMF under argon. The bromide derivative compound 3 (3.5 g, 4.6 mmol, 1.1 eq.) was dissolved in DMF (10 mL) and added to the reaction mixture using a syringe. The reaction mixture was stirred at ambient temperature for 1 h. The solution was then quenched with 5% aqueous acetic acid and diluted with water. The desired product was extracted in dichloromethane, dried and concentrated. The oily product 5 was purified by flash chromatography (solid loading) on normal phase using EtOAc and hexanes as eluents to give a red solid (5 g, 71% yield). M+H calc. 761.20, M+H obs. 761.34; ¹H NMR (CDCl₃) δ: 1.58 (m, 11H, Boc+CH₂); 1.84 (m, 1H); 1.96 (m, 1H); 2.24 (m, 2H, CH₂); 3.00 (m, 1H, CH_{α}); 3.22 (m, 2H, CH₂); 3.45 and 4.25 (AB system, 2H, CH₂ (benzyl), J=12.8 Hz); 4.27 (m, 1H, CH_{α}); 6.65 (d, 2H); 6.88 (d, 1H); 7.07 (m, 2H); 7.14 (m, 2H); 7.28 (m, 3H); 7.35-7.39 (m, 2H); 7.52 (m, 2H); 7.96 (d, 2H); 8.28 (m, 2H).

### Fmoc-αMe-6Cl-Trp(Boc)-OH, 6

To a solution of 3N HCl/MeOH (1/3, 15 mL) at 50° C. was added a solution of compound 4 (1.75 g, 2.3 mmol, 1 eq.) in MeOH (5 ml) dropwise. The starting material disappeared within 3-4 h. The acidic solution was then cooled to 0° C. with an ice bath and quenched with an aqueous solution of Na₂CO₃ (1.21 g, 11.5 mmol, 5 eq.). Methanol was removed and 8 eq. of Na₂CO₃ (1.95 g, 18.4 mmol) were added to the suspension. EDTA disodium salt dihydrate (1.68 g, 4.5 mmol, 2 eq.) was then added, and the resulting suspension was stirred for 2 h. A solution of Fmoc-OSu (0.84 g, 2.5 mmol, 1.1 eq.) in acetone (50 mL) was added, and the reaction was stirred overnight. The reaction was diluted with diethyl ether and 1N HCl. The organic layer was then dried over magnesium sulfate and concentrated in vacuo. The desired product 6 was purified on normal phase using acetone and dichloromethane as eluents to give a white foam (0.9 g, 70% yield). M+H calc. 575.19, M+H obs. 575.37; ¹H NMR (CDCl₃) 1.59 (s, 9H, Boc); 1.68 (s, 3H, Me); 3.48 (bs, 2H, CH₂); 4.22 (m, 1H, CH); 4.39 (bs, 2H, CH₂); 5.47 (s, 1H, NH); 7.10 (m, 1H); 7.18 (m, 2H); 7.27 (m, 2H); 7.39 (m, 2H); 7.50 (m, 2H); 7.75 (d, 2H); 8.12 (bs, 1H).

### Fmoc-6Cl-Trp(Boc)-OH, 7

To a solution of 3N HCl/MeOH (1/3, 44 mL) at 50° C. was added a solution of compound 5 (5 g, 6.6 mmol, 1 eq.) in MeOH (10 ml) dropwise. The starting material disappeared within 3-4 h. The acidic solution was then cooled to 0° C. with an ice bath and quenched with an aqueous solution of Na₂CO₃ (3.48 g, 33 mmol, 5 eq.). Methanol was removed and 8 eq. of Na₂CO₃ (5.57 g, 52 mmol) were added to the suspension. EDTA disodium salt dihydrate (4.89 g, 13.1 mmol, 2 eq.) was added to the suspension, and the resulting suspension was stirred for 2 h. A solution of Fmoc-OSu (2.21 g, 6.55 mmol, 1.1 eq.) in acetone (100 mL) was added, and the reaction was stirred overnight. The reaction was diluted with diethyl ether and 1N HCl. The organic layer was then dried over magnesium sulfate and concentrated in vacuo. The desired product 7 was purified on normal phase using acetone and dichloromethane as eluents to give a white foam (2.6 g, 69% yield). M+H calc. 561.17, M+H obs. 561.37; ¹H NMR (CDCl₃) 1.63 (s, 9H, Boc); 3.26 (m, 2H, CH₂); 4.19 (m, 1H, CH); 4.39 (m, 2H, CH₂); 4.76 (m, 1H); 5.35 (d, 1H, NH); 7.18 (m, 2H); 7.28 (m, 2H); 7.39 (m, 3H); 7.50 (m, 2H); 7.75 (d, 2H); 8.14 (bs, 1H).

### Example 2: Peptidomimetic Macrocycles

Peptidomimetic macrocycles were designed by replacing two or more naturally-occurring amino acids with the corresponding synthetic amino acids. Substitutions were made at i and i+4, and i and i+7 positions. Peptide synthesis was performed manually or using an automated peptide synthesizer under solid phase conditions using rink amide AM resin and Fmoc main-chain protecting group chemistry. For the coupling of natural Fmoc-protected amino acids, 10 eq. of amino acid and a 1:1:2 molar ratio of coupling reagents HBTU/HOBt/DIEA were employed. Non-natural amino acids (4 eq.) were coupled with a 1:1:2 molar ratio of HATU/HOBt/DIEA. The N-termini of the synthetic peptides were acetylated, and the C-termini were amidated.

Purification of crosslinked compounds was achieved by HPLC on a reverse phase C18 column to yield the pure compounds. The chemical compositions of the pure products were confirmed by LC/MS mass spectrometry and amino acid analysis.

Synthesis of Dialkyne-Crosslinked Peptidomimetic Macrocycles, Including SP662, SP663 and SP664.

Fully protected resin-bound peptides were synthesized on a PEG-PS resin (loading 0.45 mmol/g) on a 0.2 mmol scale. Deprotection of the temporary Fmoc group was achieved by 3×10 min treatments of the resin bound peptide with 20% (v/v) piperidine in DMF. After washing with NMP (3×), dichloromethane (3×) and NMP (3×), coupling of each successive amino acid was achieved with 1×60 min incubation with the appropriate pre-activated Fmoc-amino acid derivative. All protected amino acids (0.4 mmol) were dissolved in NMP and activated with HCTU (0.4 mmol) and DIEA (0.8 mmol) prior to transfer of the coupling solution to the de-protected resin-bound peptide. After coupling was completed, the resin was washed in preparation for the next deprotection/coupling cycle.

Acetylation of the amino terminus was carried out in the presence of acetic anhydride/DIEA in NMP. The LC-MS analysis of a cleaved and de-protected sample obtained from an aliquot of the fully assembled resin-bound peptide was accomplished in order to verifying the completion of each coupling. In a typical example, tetrahydrofuran (4 ml) and triethylamine (2 ml) were added to the peptide resin (0.2 mmol) in a 40 ml glass vial and shaken for 10 minutes. Pd(PPh₃)₂Cl₂ (0.014 g, 0.02 mmol) and copper iodide (0.008 g, 0.04 mmol) were then added and the resulting reaction mixture was mechanically shaken 16 hours while open to atmosphere. The diyne-cyclized resin-bound peptides were de-protected and cleaved from the solid support by treatment with TFA/H₂O/TIS (95/5/5 v/v) for 2.5 h at room temperature. After filtration of the resin the TFA solution was precipitated in cold diethyl ether and centrifuged to yield the desired product as a solid. The crude product was purified by preparative HPLC.

Synthesis of Single Alkyne-Crosslinked Peptidomimetic Macrocycles, Including SP665.

Fully protected resin-bound peptides were synthesized on a Rink amide MBHA resin (loading 0.62 mmol/g) on a 0.1 mmol scale. Deprotection of the temporary Fmoc group was achieved by 2×20 min treatments of the resin bound peptide with 25% (v/v) piperidine in NMP. After extensive flow washing with NMP and dichloromethane, coupling of each successive amino acid was achieved with 1×60 min incubation with the appropriate pre-activated Fmoc-amino acid derivative. All protected amino acids (1 mmol) were dissolved in NMP and activated with HCTU (1 mmol) and DIEA (1 mmol) prior to transfer of the coupling solution to the de-protected resin-bound peptide. After coupling was completed, the resin was extensively flow washed in preparation for the next deprotection/coupling cycle.

Acetylation of the amino terminus was carried out in the presence of acetic anhydride/DIEA in NMP/NMM. The LC-MS analysis of a cleaved and de-protected sample obtained from an aliquot of the fully assembled resin-bound peptide was accomplished to verify the completion of each coupling reaction. In a typical example, the peptide resin (0.1 mmol) was washed with DCM. Resin was loaded into a microwave vial. The vessel was evacuated and purged with nitrogen. Molybdenum hexacarbonyl (0.01 eq.) was added. Anhydrous chlorobenzene was added to the reaction vessel. Then 2-fluorophenol (1 eq.) was added. The reaction was then loaded into the microwave and held at 130° C. for 10 minutes. The reaction pushed for a longer period time when needed to complete the reaction. The alkyne-metathesized resin-bound peptides were de-protected and cleaved from the solid support by treating the solid support with TFA/H₂O/TIS (94/3/3 v/v) for 3 h at room temperature. After filtration of the resin, the TFA solution was precipitated in cold diethyl ether and centrifuged to yield the desired product as a solid. The crude product was purified by preparative HPLC.

TABLE 1
TABLE 1 shows a list of peptidomimetic macrocycles prepared.

| | | SEQ | | | | Calc | Calc | Calc |
|---|---|---|---|---|---|---|---|---|
| | | ID | Iso- | Exact | Found | (M + | (M + | (M + |
| SP | Sequence | NO: | mer | Mass | Mass | 1)/1 | 2)/2 | 3)/3 |
| 1 | Ac-F$r8AYWEAc3cL$AAA-NH₂ | 10 | | 1456.78 | 729.44 | 1457.79 | 729.4 | 486.6 |
| 2 | Ac-F$r8AYWEAc3cL$AAibA-NH₂ | 11 | | 1470.79 | 736.4 | 1471.8 | 736.4 | 491.27 |
| 3 | Ac-LTF$r8AYWAQL$SANle-NH₂ | 12 | | 1715.97 | 859.02 | 1716.98 | 858.99 | 573 |
| 4 | Ac-LTF$r8AYWAQL$SAL-NH₂ | 13 | | 1715.97 | 859.02 | 1716.98 | 858.99 | 573 |
| 5 | Ac-LTF$r8AYWAQL$SAM-NH₂ | 14 | | 1733.92 | 868.48 | 1734.93 | 867.97 | 578.98 |
| 6 | Ac-LTF$r8AYWAQL$SAhL-NH₂ | 15 | | 1729.98 | 865.98 | 1730.99 | 866 | 577.67 |
| 7 | Ac-LTF$r8AYWAQL$SAF-NH₂ | 16 | | 1749.95 | 876.36 | 1750.96 | 875.98 | 584.32 |
| 8 | Ac-LTF$r8AYWAQL$SAI-NH₂ | 17 | | 1715.97 | 859.02 | 1716.98 | 858.99 | 573 |
| 9 | Ac-LTF$r8AYWAQL$SAChg-NH₂ | 18 | | 1741.98 | 871.98 | 1742.99 | 872 | 581.67 |
| 10 | Ac-LTF$r8AYWAQL$SAAib-NH₂ | 19 | | 1687.93 | 845.36 | 1688.94 | 844.97 | 563.65 |
| 11 | Ac-LTF$r8AYWAQL$SAA-NH₂ | 20 | | 1673.92 | 838.01 | 1674.93 | 837.97 | 558.98 |
| 12 | Ac-LTF$r8AYWA$L$S$Nle-NH₂ | 21 | | 1767.04 | 884.77 | 1768.05 | 884.53 | 590.02 |
| 13 | Ac-LTF$r8AYWA$L$S$A-NH₂ | 22 | | 1724.99 | 864.23 | 1726 | 863.5 | 576 |
| 14 | Ac-F$r8AYWEAc3cL$AANle-NH₂ | 23 | | 1498.82 | 750.46 | 1499.83 | 750.42 | 500.61 |
| 15 | Ac-F$r8AYWEAc3cL$AAL-NH₂ | 24 | | 1498.82 | 750.46 | 1499.83 | 750.42 | 500.61 |
| 16 | Ac-F$r8AYWEAc3cL$AAM-NH₂ | 25 | | 1516.78 | 759.41 | 1517.79 | 759.4 | 506.6 |
| 17 | Ac-F$r8AYWEAc3cL$AAhL-NH₂ | 26 | | 1512.84 | 757.49 | 1513.85 | 757.43 | 505.29 |
| 18 | Ac-F$r8AYWEAc3cL$AAF-NH₂ | 27 | | 1532.81 | 767.48 | 1533.82 | 767.41 | 511.94 |
| 19 | Ac-F$r8AYWEAc3cL$AAI-NH₂ | 28 | | 1498.82 | 750.39 | 1499.83 | 750.42 | 500.61 |
| 20 | Ac-F$r8AYWEAc3cL$AAChg-NH₂ | 29 | | 1524.84 | 763.48 | 1525.85 | 763.43 | 509.29 |
| 21 | Ac-F$r8AYWEAc3cL$AACha-NH₂ | 30 | | 1538.85 | 770.44 | 1539.86 | 770.43 | 513.96 |
| 22 | Ac-F$r8AYWEAc3cL$AAAib-NH₂ | 31 | | 1470.79 | 736.84 | 1471.8 | 736.4 | 491.27 |
| 23 | Ac-LTF$r8AYWAQL$AAAibV-NH₂ | 32 | | 1771.01 | 885.81 | 1772.02 | 886.51 | 591.34 |
| 24 | Ac-LTF$r8AYWAQL$AAAibV-NH₂ | 33 | iso2 | 1771.01 | 886.26 | 1772.02 | 886.51 | 591.34 |
| 25 | Ac-LTF$r8AYWAQL$SAibAA-NH₂ | 34 | | 1758.97 | 879.89 | 1759.98 | 880.49 | 587.33 |
| 26 | Ac-LTF$r8AYWAQL$SAibAA-NH₂ | 35 | iso2 | 1758.97 | 880.34 | 1759.98 | 880.49 | 587.33 |
| 27 | Ac-HLTF$r8HHWHQL$AANleNle-NH₂ | 36 | | 2056.15 | 1028.86 | 2057.16 | 1029.08 | 686.39 |
| 28 | Ac-DLTF$r8HHWHQL$RRLV-NH₂ | 37 | | 2190.23 | 731.15 | 2191.24 | 1096.12 | 731.08 |
| 29 | Ac-HHTF$r8HHWHQL$AAML-NH₂ | 38 | | 2098.08 | 700.43 | 2099.09 | 1050.05 | 700.37 |
| 30 | Ac-F$r8HHWHQL$RRDCha-NH₂ | 39 | | 1917.06 | 959.96 | 1918.07 | 959.54 | 640.03 |
| 31 | Ac-F$r8HHWHQL$HRFV-NH₂ | 40 | | 1876.02 | 938.65 | 1877.03 | 939.02 | 626.35 |
| 32 | Ac-HLTF$r8HHWHQL$AAhLA-NH₂ | 41 | | 2028.12 | 677.2 | 2029.13 | 1015.07 | 677.05 |
| 33 | Ac-DLTF$r8HHWHQL$RRChgl-NH₂ | 42 | | 2230.26 | 1115.89 | 2231.27 | 1116.14 | 744.43 |
| 34 | Ac-DLTF$r8HHWHQL$RRChgl-NH₂ | 43 | iso2 | 2230.26 | 1115.96 | 2231.27 | 1116.14 | 744.43 |
| 35 | Ac-HHTF$r8HHWHQL$AAChav-NH₂ | 44 | | 2106.14 | 1053.95 | 2107.15 | 1054.08 | 703.05 |
| 36 | Ac-F$r8HHWHQL$RRDa-NH₂ | 45 | | 1834.99 | 918.3 | 1836 | 918.5 | 612.67 |
| 37 | Ac-F$r8HHWHQL$HRAibG-NH₂ | 46 | | 1771.95 | 886.77 | 1772.96 | 886.98 | 591.66 |
| 38 | Ac-F$r8AYWAQL$HHNleL-NH₂ | 47 | | 1730.97 | 866.57 | 1731.98 | 866.49 | 578 |
| 39 | Ac-F$r8AYWSAL$HQANle-NH₂ | 48 | | 1638.89 | 820.54 | 1639.9 | 820.45 | 547.3 |
| 40 | Ac-F$r8AYWVQL$QHChgl-NH₂ | 49 | | 1776.01 | 889.44 | 1777.02 | 889.01 | 593.01 |
| 41 | Ac-F$r8AYWTAL$QQNlev-NH₂ | 50 | | 1671.94 | 836.97 | 1672.95 | 836.98 | 558.32 |
| 42 | Ac-F$r8AYWYQL$HAibAa-NH₂ | 51 | | 1686.89 | 844.52 | 1687.9 | 844.45 | 563.3 |
| 43 | Ac-LTF$r8AYWAQL$HHLa-NH₂ | 52 | | 1903.05 | 952.27 | 1904.06 | 952.53 | 635.36 |
| 44 | Ac-LTF$r8AYWAQL$HHLa-NH₂ | 53 | iso2 | 1903.05 | 952.27 | 1904.06 | 952.53 | 635.36 |
| 45 | Ac-LTF$r8AYWAQL$HQNlev-NH₂ | 54 | | 1922.08 | 962.48 | 1923.09 | 962.05 | 641.7 |
| 46 | Ac-LTF$r8AYwAQL$HQNlev-NH₂ | 55 | iso2 | 1922.08 | 962.4 | 1923.09 | 962.05 | 641.7 |
| 47 | Ac-LTF$r8AYWAQL$QQM1-NH₂ | 56 | | 1945.05 | 973.95 | 1946.06 | 973.53 | 649.36 |
| 48 | Ac-LTF$r8AYWAQL$QQM1-NH₂ | 57 | iso2 | 1945.05 | 973.88 | 1946.06 | 973.53 | 649.36 |
| 49 | Ac-LTF$r8AYWAQL$HAibhLV-NH₂ | 58 | | 1893.09 | 948.31 | 1894.1 | 947.55 | 632.04 |
| 50 | Ac-LTF$r8AYWAQL$AHFA-NH₂ | 59 | | 1871.01 | 937.4 | 1872.02 | 936.51 | 624.68 |
| 51 | Ac-HLTF$r8HHWHQL$AANlel-NH₂ | 60 | | 2056.15 | 1028.79 | 2057.16 | 1029.08 | 686.39 |
| 52 | Ac-DLTF$r8HHWHQL$RRLa-NH₂ | 61 | | 2162.2 | 721.82 | 2163.21 | 1082.11 | 721.74 |
| 53 | Ac-HHTF$r8HHWHQL$AAMv-NH₂ | 62 | | 2084.07 | 1042.92 | 2085.08 | 1043.04 | 695.7 |
| 54 | Ac-F$r8HHWHQL$RRDA-NH₂ | 63 | | 1834.99 | 612.74 | 1836 | 918.5 | 612.67 |
| 55 | Ac-F$r8HHWHQL$HRFCha-NH₂ | 64 | | 1930.06 | 966.47 | 1931.07 | 966.04 | 644.36 |
| 56 | Ac-F$r8AYWEAL$AA-NHAm | 65 | | 1443.82 | 1445.71 | 1444.83 | 722.92 | 482.28 |
| 57 | Ac-F$r8AYWEAL$AA-NHiAm | 66 | | 1443.82 | 723.13 | 1444.83 | 722.92 | 482.28 |
| 58 | Ac-F$r8AYWEAL$AA-NHnPr3Ph | 67 | | 1491.82 | 747.3 | 1492.83 | 746.92 | 498.28 |
| 59 | Ac-F$r8AYWEAL$AA-NHnBu33Me | 68 | | 1457.83 | 1458.94 | 1458.84 | 729.92 | 486.95 |
| 60 | Ac-F$r8AYWEAL$AA-NHnPr | 69 | | 1415.79 | 709.28 | 1416.8 | 708.9 | 472.94 |
| 61 | Ac-F$r8AYWEAL$AA-NHnEt2Ch | 70 | | 1483.85 | 1485.77 | 1484.86 | 742.93 | 495.62 |
| 62 | Ac-F$r8AYWEAL$AA-NHnEt2Cp | 71 | | 1469.83 | 1470.78 | 1470.84 | 735.92 | 490.95 |
| 63 | Ac-F$r8AYWEAL$AA-NHHex | 72 | | 1457.83 | 730.19 | 1458.84 | 729.92 | 486.95 |
| 64 | Ac-LTF$r8AYWAQL$AAIA-NH₂ | 73 | | 1771.01 | 885.81 | 1772.02 | 886.51 | 591.34 |
| 65 | Ac-LTF$r8AYWAQL$AAIA-NH₂ | 74 | iso2 | 1771.01 | 866.8 | 1772.02 | 886.51 | 591.34 |
| 66 | Ac-LTF$r8AYWAAL$AAMA-NH₂ | 75 | | 1731.94 | 867.08 | 1732.95 | 866.98 | 578.32 |
| 67 | Ac-LTF$r8AYWAAL$AAMA-NH₂ | 76 | iso2 | 1731.94 | 867.28 | 1732.95 | 866.98 | 578.32 |
| 68 | Ac-LTF$r8AYwAQL$AANleA-NH₂ | 77 | | 1771.01 | 867.1 | 1772.02 | 886.51 | 591.34 |
| 69 | Ac-LTF$r8AYWAQL$AANleA-NH₂ | 78 | iso2 | 1771.01 | 886.89 | 1772.02 | 886.51 | 591.34 |
| 70 | Ac-LTF$r8AYWAQL$AAIa-NH₂ | 79 | | 1771.01 | 886.8 | 1772.02 | 886.51 | 591.34 |
| 71 | Ac-LTF$r8AYWAQL$AAIa-NH₂ | 80 | iso2 | 1771.01 | 887.09 | 1772.02 | 886.51 | 591.34 |
| 72 | Ac-LTF$r8AYWAAL$AAMa-NH₂ | 81 | | 1731.94 | 867.17 | 1732.95 | 866.98 | 578.32 |
| 73 | Ac-LTF$r8AYWAAL$AAMa-NH₂ | 82 | iso2 | 1731.94 | 867.37 | 1732.95 | 866.98 | 578.32 |
| 74 | Ac-LTF$r8AYWAQL$AANlea-NH₂ | 83 | | 1771.01 | 887.08 | 1772.02 | 886.51 | 591.34 |
| 75 | Ac-LTF$r8AYWAQL$AANlea-NH₂ | 84 | iso2 | 1771.01 | 887.08 | 1772.02 | 886.51 | 591.34 |
| 76 | Ac-LTF$r8AYWAAL$AAIv-NH₂ | 85 | | 1742.02 | 872.37 | 1743.03 | 872.02 | 581.68 |
| 77 | Ac-LTF$r8AYWAAL$AAIv-NH₂ | 86 | iso2 | 1742.02 | 872.74 | 1743.03 | 872.02 | 581.68 |
| 78 | Ac-LTF$r8AYWAQL$AAMv-NH₂ | 87 | | 1817 | 910.02 | 1818.01 | 909.51 | 606.67 |
| 79 | Ac-LTF$r8AYWAAL$AANlev-NH₂ | 88 | | 1742.02 | 872.37 | 1743.03 | 872.02 | 581.68 |
| 80 | Ac-LTF$r8AYWAAL$AANlev-NH₂ | 89 | iso2 | 1742.02 | 872.28 | 1743.03 | 872.02 | 581.68 |
| 81 | Ac-LTF$r8AYWAQL$AAI1-NH₂ | 90 | | 1813.05 | 907.81 | 1814.06 | 907.53 | 605.36 |
| 82 | Ac-LTF$r8AYWAQL$AAIl-NH₂ | 91 | iso2 | 1813.05 | 907.81 | 1814.06 | 907.53 | 605.36 |
| 83 | Ac-LTF$r8AYWAAL$AAMl-NH₂ | 92 | | 1773.99 | 887.37 | 1775 | 888 | 592.34 |
| 84 | Ac-LTF$r8AYWAQL$AANlel-NH₂ | 93 | | 1813.05 | 907.61 | 1814.06 | 907.53 | 605.36 |
| 85 | Ac-LTF$r8AYWAQL$AANlel-NH₂ | 94 | iso2 | 1813.05 | 907.71 | 1814.06 | 907.53 | 605.36 |
| 86 | Ac-F$r8AYWEAL$AAMA-NH₂ | 95 | | 1575.82 | 789.02 | 1576.83 | 788.92 | 526.28 |
| 87 | Ac-F$r8AYWEAL$AANleA-NH₂ | 96 | | 1557.86 | 780.14 | 1558.87 | 779.94 | 520.29 |
| 88 | Ac-F$r8AYWEAL$AAIa-NH₂ | 97 | | 1557.86 | 780.33 | 1558.87 | 779.94 | 520.29 |
| 89 | Ac-F$r8AYWEAL$AAMa-NH₂ | 98 | | 1575.82 | 789.3 | 1576.83 | 788.92 | 526.28 |
| 90 | Ac-F$r8AYWEAL$AANlea-NH₂ | 99 | | 1557.86 | 779.4 | 1558.87 | 779.94 | 520.29 |
| 91 | Ac-F$r8AYWEAL$AAIv-NH₂ | 100 | | 1585.89 | 794.29 | 1586.9 | 793.95 | 529.64 |
| 92 | Ac-F$r8AYwEAL$AAmv-NH₂ | 101 | | 1603.85 | 803.08 | 1604.86 | 802.93 | 535.62 |
| 93 | Ac-F$r8AYWEAL$AANlev-NH₂ | 102 | | 1585.89 | 793.46 | 1586.9 | 793.95 | 529.64 |
| 94 | Ac-F$r8AYWEAL$AAIl-NH₂ | 103 | | 1599.91 | 800.49 | 1600.92 | 800.96 | 534.31 |
| 95 | Ac-F$r8AYWEAL$AAMl-NH₂ | 104 | | 1617.86 | 809.44 | 1618.87 | 809.94 | 540.29 |
| 96 | Ac-F$r8AYWEAL$AANlel-NH₂ | 105 | | 1599.91 | 801.7 | 1600.92 | 800.96 | 534.31 |
| 97 | Ac-F$r8AYWEAL$AANlel-NH₂ | 106 | iso2 | 1599.91 | 801.42 | 1600.92 | 800.96 | 534.31 |
| 98 | Ac-LTF$r8AY6clWAQLSAA-NH₂ | 107 | | 1707.88 | 855.72 | 1708.89 | 854.95 | 570.3 |
| 99 | Ac-LTF$r8AY6clWAQLSAA-NH₂ | 108 | iso2 | 1707.88 | 855.35 | 1708.89 | 854.95 | 570.3 |
| 100 | Ac-WTF$r8FYWSQL$AVAa-NH₂ | 109 | | 1922.01 | 962.21 | 1923.02 | 962.01 | 641.68 |
| 101 | Ac-WTF$r8FYWSQL$AVAa-NH₂ | 110 | iso2 | 1922.01 | 962.49 | 1923.02 | 962.01 | 641.68 |
| 102 | Ac-WTF$r8VYWSQL$AVA-NH₂ | 111 | | 1802.98 | 902.72 | 1803.99 | 902.5 | 602 |
| 103 | Ac-WTF$r8VYWSQL$AVA-NH₂ | 112 | iso2 | 1802.98 | 903 | 1803.99 | 902.5 | 602 |
| 104 | Ac-WTF$r8FYWSQL$SAAa-NH₂ | 113 | | 1909.98 | 956.47 | 1910.99 | 956 | 637.67 |
| 105 | Ac-WTF$r8FYwSQL$SAAa-NH₂ | 114 | iso2 | 1909.98 | 956.47 | 1910.99 | 956 | 637.67 |
| 106 | Ac-WTF$r8VYWSQL$AVAaa-NH₂ | 115 | | 1945.05 | 974.15 | 1946.06 | 973.53 | 649.36 |
| 107 | Ac-WTF$r8VYWSQL$AVAaa-NH₂ | 116 | iso2 | 1945.05 | 973.78 | 1946.06 | 973.53 | 649.36 |
| 108 | Ac-LTF$r8AYWAQL$AVG-NH₂ | 117 | | 1671.94 | 837.52 | 1672.95 | 836.98 | 558.32 |
| 109 | Ac-LTF$r8AYWAQL$AVG-NH₂ | 118 | iso2 | 1671.94 | 837.21 | 1672.95 | 836.98 | 558.32 |
| 110 | Ac-LTF$r8AYWAQL$AVQ-NH₂ | 119 | | 1742.98 | 872.74 | 1743.99 | 872.5 | 582 |
| 111 | Ac-LTF$r8AYWAQL$AVQ-NH₂ | 120 | iso2 | 1742.98 | 872.74 | 1743.99 | 872.5 | 582 |
| 112 | Ac-LTF$r8AYWAQL$SAa-NH₂ | 121 | | 1673.92 | 838.23 | 1674.93 | 837.97 | 558.98 |
| 113 | Ac-LTF$r8AYWAQL$SAa-NH₂ | 122 | iso2 | 1673.92 | 838.32 | 1674.93 | 837.97 | 558.98 |
| 114 | Ac-LTF$r8AYWAQhL$SAA-NH₂ | 123 | | 1687.93 | 844.37 | 1688.94 | 844.97 | 563.65 |
| 115 | Ac-LTF$r8AYWAQhL$SAA-NH₂ | 124 | iso2 | 1687.93 | 844.81 | 1688.94 | 844.97 | 563.65 |
| 116 | Ac-LTF$r8AYWEQLStSA$-NH₂ | 125 | | 1826 | 905.27 | 1827.01 | 914.01 | 609.67 |
| 117 | Ac-LTF$r8AYWAQL$SLA-NH₂ | 126 | | 1715.97 | 858.48 | 1716.98 | 858.99 | 573 |
| 118 | Ac-LTF$r8AYWAQL$SLA-NH₂ | 127 | iso2 | 1715.97 | 858.87 | 1716.98 | 858.99 | 573 |
| 119 | Ac-LTF$r8AYWAQL$SWA-NH₂ | 128 | | 1788.96 | 895.21 | 1789.97 | 895.49 | 597.33 |
| 120 | Ac-LTF$r8AYWAQL$SWA-NH₂ | 129 | iso2 | 1788.96 | 895.28 | 1789.97 | 895.49 | 597.33 |
| 121 | Ac-LTF$r8AYWAQL$SVS-NH₂ | 130 | | 1717.94 | 859.84 | 1718.95 | 859.98 | 573.65 |
| 122 | Ac-LTF$r8AYWAQL$SAS-NH₂ | 131 | | 1689.91 | 845.85 | 1690.92 | 845.96 | 564.31 |
| 123 | Ac-LTF$r8AYWAQL$SVG-NH₂ | 132 | | 1687.93 | 844.81 | 1688.94 | 844.97 | 563.65 |
| 124 | Ac-ETF$r8VYWAQL$SAa-NH₂ | 133 | | 1717.91 | 859.76 | 1718.92 | 859.96 | 573.64 |
| 125 | Ac-ETF$r8VYWAQL$SAA-NH₂ | 134 | | 1717.91 | 859.84 | 1718.92 | 859.96 | 573.64 |
| 126 | Ac-ETF$r8VYWAQL$SVA-NH₂ | 135 | | 1745.94 | 873.82 | 1746.95 | 873.98 | 582.99 |
| 127 | Ac-ETF$r8VYWAQL$SLA-NH₂ | 136 | | 1759.96 | 880.85 | 1760.97 | 880.99 | 587.66 |
| 128 | Ac-ETF$r8VYWAQL$SWA-NH₂ | 137 | | 1832.95 | 917.34 | 1833.96 | 917.48 | 611.99 |
| 129 | Ac-ETF$r8KYWAQL$SWA-NH₂ | 138 | | 1861.98 | 931.92 | 1862.99 | 932 | 621.67 |
| 130 | Ac-ETF$r8VYWAQL$SVS-NH₂ | 139 | | 1761.93 | 881.89 | 1762.94 | 881.97 | 588.32 |
| 131 | Ac-ETF$r8VYWAQL$SAS-NH₂ | 140 | | 1733.9 | 867.83 | 1734.91 | 867.96 | 578.97 |
| 132 | Ac-ETF$r8VYWAQL$SVG-NH₂ | 141 | | 1731.92 | 866.87 | 1732.93 | 866.97 | 578.31 |
| 133 | Ac-LTF$r8VYWAQL$SSa-NH₂ | 142 | | 1717.94 | 859.47 | 1718.95 | 859.98 | 573.65 |
| 134 | Ac-ETF$r8VYWAQL$SSa-NH₂ | 143 | | 1733.9 | 867.83 | 1734.91 | 867.96 | 578.97 |
| 135 | Ac-LTF$r8VYWAQL$SNa-NH₂ | 144 | | 1744.96 | 873.38 | 1745.97 | 873.49 | 582.66 |
| 136 | Ac-ETF$r8VYWAQL$SNa-NH₂ | 145 | | 1760.91 | 881.3 | 1761.92 | 881.46 | 587.98 |
| 137 | Ac-LTF$r8VYWAQL$SAa-NH₂ | 146 | | 1701.95 | 851.84 | 1702.96 | 851.98 | 568.32 |
| 138 | Ac-LTF$r8VYWAQL$SVA-NH₂ | 147 | | 1729.98 | 865.53 | 1730.99 | 866 | 577.67 |
| 139 | Ac-LTF$r8VYWAQL$SVA-NH₂ | 148 | iso2 | 1729.98 | 865.9 | 1730.99 | 866 | 577.67 |
| 140 | Ac-LTF$r8VYWAQL$SWA-NH₂ | 149 | | 1816.99 | 909.42 | 1818 | 909.5 | 606.67 |
| 141 | Ac-LTF$r8VYWAQL$SVS-NH₂ | 150 | | 1745.98 | 873.9 | 1746.99 | 874 | 583 |
| 142 | Ac-LTF$r8VYWAQL$SVS-NH₂ | 151 | iso2 | 1745.98 | 873.9 | 1746.99 | 874 | 583 |
| 143 | Ac-LTF$r8VYWAQL$SAS-NH₂ | 152 | | 1717.94 | 859.84 | 1718.95 | 859.98 | 573.65 |
| 144 | Ac-LTF$r8VYWAQL$SAS-NH₂ | 153 | iso2 | 1717.94 | 859.91 | 1718.95 | 859.98 | 573.65 |
| 145 | Ac-LTF$r8VYWAQL$SVG-NH₂ | 154 | | 1715.97 | 858.87 | 1716.98 | 858.99 | 573 |
| 146 | Ac-LTF$r8VYWAQL$SVG-NH₂ | 155 | iso2 | 1715.97 | 858.87 | 1716.98 | 858.99 | 573 |
| 147 | Ac-LTF$r8EYWAQCha$SAA-NH₂ | 156 | | 1771.96 | 886.85 | 1772.97 | 886.99 | 591.66 |
| 148 | Ac-LTF$r8EYWAQCha$SAA-NH₂ | 157 | iso2 | 1771.96 | 886.85 | 1772.97 | 886.99 | 591.66 |
| 149 | Ac-LTF$r8EYWAQCpg$SAA-NH₂ | 158 | | 1743.92 | 872.86 | 1744.93 | 872.97 | 582.31 |
| 150 | Ac-LTF$r8EYWAQCpg$SAA-NH₂ | 159 | iso2 | 1743.92 | 872.86 | 1744.93 | 872.97 | 582.31 |
| 151 | Ac-LTF$r8EYWAQF$SAA-NH₂ | 160 | | 1765.91 | 883.44 | 1766.92 | 883.96 | 589.64 |
| 152 | Ac-LTF$r8EYWAQF$SAA-NH₂ | 161 | iso2 | 1765.91 | 883.89 | 1766.92 | 883.96 | 589.64 |
| 153 | Ac-LTF$r8EYWAQCba$SAA-NH₂ | 162 | | 1743.92 | 872.42 | 1744.93 | 872.97 | 582.31 |
| 154 | Ac-LTF$r8EYWAQCba$SAA-NH₂ | 163 | iso2 | 1743.92 | 873.39 | 1744.93 | 872.97 | 582.31 |
| 155 | Ac-LTF3Cl$r8EYWAQL$SAA-NH₂ | 164 | | 1765.89 | 883.89 | 1766.9 | 883.95 | 589.64 |
| 156 | Ac-LTF3Cl$r8EYWAQL$SAA-NH₂ | 165 | iso2 | 1765.89 | 883.96 | 1766.9 | 883.95 | 589.64 |
| 157 | Ac-LTF34F2$r8EYWAQL$SAA-NH₂ | 166 | | 1767.91 | 884.48 | 1768.92 | 884.96 | 590.31 |
| 158 | Ac-LTF34F2$r8EYWAQL$SAA-NH₂ | 167 | iso2 | 1767.91 | 884.48 | 1768.92 | 884.96 | 590.31 |
| 159 | Ac-LTF34F2$r8EYWAQhL$SAA-NH₂ | 168 | | 1781.92 | 891.44 | 1782.93 | 891.97 | 594.98 |
| 160 | Ac-LTF34F2$r8EYWAQhL$SAA-NH₂ | 169 | iso2 | 1781.92 | 891.88 | 1782.93 | 891.97 | 594.98 |
| 161 | Ac-ETF$r8EYWAQL$SAA-NH₂ | 170 | | 1747.88 | 874.34 | 1748.89 | 874.95 | 583.63 |
| 162 | Ac-LTF$r8AYWVQL$SAA-NH₂ | 171 | | 1701.95 | 851.4 | 1702.96 | 851.98 | 568.32 |
| 163 | Ac-LTF$r8AHWAQL$SAA-NH₂ | 172 | | 1647.91 | 824.83 | 1648.92 | 824.96 | 550.31 |
| 164 | Ac-LTF$r8AEWAQL$SAA-NH₂ | 173 | | 1639.9 | 820.39 | 1640.91 | 820.96 | 547.64 |
| 165 | Ac-LTF$r8ASWAQL$SAA-NH₂ | 174 | | 1597.89 | 799.38 | 1598.9 | 799.95 | 533.64 |
| 166 | Ac-LTF$r8AEWAQL$SAA-NH₂ | 175 | iso2 | 1639.9 | 820.39 | 1640.91 | 820.96 | 547.64 |
| 167 | Ac-LTF$r8ASWAQL$SAA-NH₂ | 176 | iso2 | 1597.89 | 800.31 | 1598.9 | 799.95 | 533.64 |
| 168 | Ac-LTF$r8AF4coohWAQL$SAA-NH₂ | 177 | | 1701.91 | 851.4 | 1702.92 | 851.96 | 568.31 |
| 169 | Ac-LTF$r8AF4coohWAQL$SAA-NH₂ | 178 | iso2 | 1701.91 | 851.4 | 1702.92 | 851.96 | 568.31 |
| 170 | Ac-LTF$r8AHWAQL$AAIa-NH₂ | 179 | | 1745 | 874.13 | 1746.01 | 873.51 | 582.67 |
| 171 | Ac-ITF$r8FYWAQL$AAIa-NH₂ | 180 | | 1847.04 | 923.92 | 1848.05 | 924.53 | 616.69 |
| 172 | Ac-ITF$r8EHWAQL$AAIa-NH₂ | 181 | | 1803.01 | 903.17 | 1804.02 | 902.51 | 602.01 |
| 173 | Ac-ITF$r8EHWAQL$AAIa-NH₂ | 182 | iso2 | 1803.01 | 903.17 | 1804.02 | 902.51 | 602.01 |
| 174 | Ac-ETF$r8EHWAQL$AAIa-NH₂ | 183 | | 1818.97 | 910.76 | 1819.98 | 910.49 | 607.33 |
| 175 | Ac-ETF$r8EHWAQL$AAIa-NH₂ | 184 | iso2 | 1818.97 | 910.85 | 1819.98 | 910.49 | 607.33 |
| 176 | Ac-LTF$r8AHWVQL$AAIa-NH₂ | 185 | | 1773.03 | 888.09 | 1774.04 | 887.52 | 592.02 |
| 177 | Ac-ITF$r8FYWVQL$AAIa-NH₂ | 186 | | 1875.07 | 939.16 | 1876.08 | 938.54 | 626.03 |
| 178 | Ac-ITF$r8EYWVQL$AAIa-NH₂ | 187 | | 1857.04 | 929.83 | 1858.05 | 929.53 | 620.02 |
| 179 | Ac-ITF$r8EHWVQL$AAIa-NH₂ | 188 | | 1831.04 | 916.86 | 1832.05 | 916.53 | 611.35 |
| 180 | Ac-LTF$r8AEWAQL$AAIa-NH₂ | 189 | | 1736.99 | 869.87 | 1738 | 869.5 | 580 |
| 181 | Ac-LTF$r8AF4coohWAQL$AAIa-NH₂ | 190 | | 1799 | 900.17 | 1800.01 | 900.51 | 600.67 |
| 182 | Ac-LTF$r8AF4coohWAQL$AAIa-NH₂ | 191 | iso2 | 1799 | 900.24 | 1800.01 | 900.51 | 600.67 |
| 183 | Ac-LTF$r8AHWAQL$AHFA-NH₂ | 192 | | 1845.01 | 923.89 | 1846.02 | 923.51 | 616.01 |
| 184 | Ac-ITF$r8FYWAQL$AHFA-NH₂ | 193 | | 1947.05 | 975.05 | 1948.06 | 974.53 | 650.02 |
| 185 | Ac-ITF$r8FYWAQL$AHFA-NH₂ | 194 | iso2 | 1947.05 | 976.07 | 1948.06 | 974.53 | 650.02 |
| 186 | Ac-ITF$r8FHWAQL$AEFA-NH₂ | 195 | | 1913.02 | 958.12 | 1914.03 | 957.52 | 638.68 |
| 187 | Ac-ITF$r8FHWAQL$AEFA-NH₂ | 196 | iso2 | 1913.02 | 957.86 | 1914.03 | 957.52 | 638.68 |
| 188 | Ac-ITF$r8EHWAQL$AHFA-NH₂ | 197 | | 1903.01 | 952.94 | 1904.02 | 952.51 | 635.34 |
| 189 | Ac-ITF$r8EHWAQL$AHFA-NH₂ | 198 | iso2 | 1903.01 | 953.87 | 1904.02 | 952.51 | 635.34 |
| 190 | Ac-LTF$r8AHWVQL$AHFA-NH₂ | 199 | | 1873.04 | 937.86 | 1874.05 | 937.53 | 625.35 |
| 191 | Ac-ITF$r8FYWVQL$AHFA-NH₂ | 200 | | 1975.08 | 988.83 | 1976.09 | 988.55 | 659.37 |
| 192 | Ac-ITF$r8EYWVQL$AHFA-NH₂ | 201 | | 1957.05 | 979.35 | 1958.06 | 979.53 | 653.36 |
| 193 | Ac-ITF$r8EHWVQL$AHFA-NH₂ | 202 | | 1931.05 | 967 | 1932.06 | 966.53 | 644.69 |
| 194 | Ac-ITF$r8EHWVQL$AHFA-NH₂ | 203 | iso2 | 1931.05 | 967.93 | 1932.06 | 966.53 | 644.69 |
| 195 | Ac-ETF$r8EYWAAL$SAA-NH₂ | 204 | | 1690.86 | 845.85 | 1691.87 | 846.44 | 564.63 |
| 196 | Ac-LTF$r8AYWVAL$SAA-NH₂ | 205 | | 1644.93 | 824.08 | 1645.94 | 823.47 | 549.32 |
| 197 | Ac-LTF$r8AHWAAL$SAA-NH₂ | 206 | | 1590.89 | 796.88 | 1591.9 | 796.45 | 531.3 |
| 198 | Ac-LTF$r8AEWAAL$SAA-NH₂ | 207 | | 1582.88 | 791.9 | 1583.89 | 792.45 | 528.63 |
| 199 | Ac-LTF$r8AEWAAL$SAA-NH₂ | 208 | iso2 | 1582.88 | 791.9 | 1583.89 | 792.45 | 528.63 |
| 200 | Ac-LTF$r8ASWAAL$SAA-NH₂ | 209 | | 1540.87 | 770.74 | 1541.88 | 771.44 | 514.63 |
| 201 | Ac-LTF$r8ASWAAL$SAA-NH₂ | 210 | iso2 | 1540.87 | 770.88 | 1541.88 | 771.44 | 514.63 |
| 202 | Ac-LTF$r8AYwAAL$AAIa-NH₂ | 211 | | 1713.99 | 857.39 | 1715 | 858 | 572.34 |
| 203 | Ac-LTF$r8AYWAAL$AAIa-NH₂ | 212 | iso2 | 1713.99 | 857.84 | 1715 | 858 | 572.34 |
| 204 | Ac-LTF$r8AYWAAL$AHFA-NH₂ | 213 | | 1813.99 | 907.86 | 1815 | 908 | 605.67 |
| 205 | Ac-LTF$r8EHWAQL$AHIa-NH₂ | 214 | | 1869.03 | 936.1 | 1870.04 | 935.52 | 624.02 |
| 206 | Ac-LTF$r8EHWAQL$AHIa-NH₂ | 215 | iso2 | 1869.03 | 937.03 | 1870.04 | 935.52 | 624.02 |
| 207 | Ac-LTF$r8AHWAQL$AHIa-NH₂ | 216 | | 1811.03 | 906.87 | 1812.04 | 906.52 | 604.68 |
| 208 | Ac-LTF$r8EYWAQL$AHIa-NH₂ | 217 | | 1895.04 | 949.15 | 1896.05 | 948.53 | 632.69 |
| 209 | Ac-LTF$r8AYWAQL$AAFa-NH₂ | 218 | | 1804.99 | 903.2 | 1806 | 903.5 | 602.67 |
| 210 | Ac-LTF$r8AYWAQL$AAFa-NH₂ | 219 | iso2 | 1804.99 | 903.28 | 1806 | 903.5 | 602.67 |
| 211 | Ac-LTF$r8AYWAQL$AAWa-NH₂ | 220 | | 1844 | 922.81 | 1845.01 | 923.01 | 615.67 |
| 212 | Ac-LTF$r8AYWAQL$AAVa-NH₂ | 221 | | 1756.99 | 878.86 | 1758 | 879.5 | 586.67 |
| 213 | Ac-LTF$r8AYWAQL$AAVa-NH₂ | 222 | iso2 | 1756.99 | 879.3 | 1758 | 879.5 | 586.67 |
| 214 | Ac-LTF$r8AYWAQL$AALa-NH₂ | 223 | | 1771.01 | 886.26 | 1772.02 | 886.51 | 591.34 |
| 215 | Ac-LTF$r8AYWAQL$AALa-NH₂ | 224 | iso2 | 1771.01 | 886.33 | 1772.02 | 886.51 | 591.34 |
| 216 | Ac-LTF$r8EYWAQL$AAIa-NH₂ | 225 | | 1829.01 | 914.89 | 1830.02 | 915.51 | 610.68 |
| 217 | Ac-LTF$r8EYWAQL$AAIa-NH₂ | 226 | iso2 | 1829.01 | 915.34 | 1830.02 | 915.51 | 610.68 |
| 218 | Ac-LTF$r8EYWAQL$AAFa-NH₂ | 227 | | 1863 | 932.87 | 1864.01 | 932.51 | 622.01 |
| 219 | Ac-LTF$r8EYWAQL$AAFa-NH₂ | 228 | iso2 | 1863 | 932.87 | 1864.01 | 932.51 | 622.01 |
| 220 | Ac-LTF$r8EYWAQL$AAVa-NH₂ | 229 | | 1815 | 908.23 | 1816.01 | 908.51 | 606.01 |
| 221 | Ac-LTF$r8EYWAQL$AAVa-NH₂ | 230 | iso2 | 1815 | 908.31 | 1816.01 | 908.51 | 606.01 |
| 222 | Ac-LTF$r8EHWAQL$AAIa-NH₂ | 231 | | 1803.01 | 903.17 | 1804.02 | 902.51 | 602.01 |
| 223 | Ac-LTF$r8EHWAQL$AAIa-NH₂ | 232 | iso2 | 1803.01 | 902.8 | 1804.02 | 902.51 | 602.01 |
| 224 | Ac-LTF$r8EHWAQL$AAWa-NH₂ | 233 | | 1876 | 939.34 | 1877.01 | 939.01 | 626.34 |
| 225 | Ac-LTF$r8EHWAQL$AAWa-NH₂ | 234 | iso2 | 1876 | 939.62 | 1877.01 | 939.01 | 626.34 |
| 226 | Ac-LTF$r8EHWAQL$AALa-NH₂ | 235 | | 1803.01 | 902.8 | 1804.02 | 902.51 | 602.01 |
| 227 | Ac-LTF$r8EHWAQL$AALa-NH₂ | 236 | iso2 | 1803.01 | 902.9 | 1804.02 | 902.51 | 602.01 |
| 228 | Ac-ETF$r8EHWVQL$AALa-NH₂ | 237 | | 1847 | 924.82 | 1848.01 | 924.51 | 616.67 |
| 229 | Ac-LTF$r8AYWAQL$AAAa-NH₂ | 238 | | 1728.96 | 865.89 | 1729.97 | 865.49 | 577.33 |
| 230 | Ac-LTF$r8AYWAQL$AAAa-NH₂ | 239 | iso2 | 1728.96 | 865.89 | 1729.97 | 865.49 | 577.33 |
| 231 | Ac-LTF$r8AYWAQL$AAAibA-NH₂ | 240 | | 1742.98 | 872.83 | 1743.99 | 872.5 | 582 |
| 232 | Ac-LTF$r8AYWAQL$AAAibA-NH₂ | 241 | iso2 | 1742.98 | 872.92 | 1743.99 | 872.5 | 582 |
| 233 | Ac-LTF$r8AYWAQL$AAAAa-NH₂ | 242 | | 1800 | 901.42 | 1801.01 | 901.01 | 601.01 |
| 234 | Ac-LTF$r5AYWAQL$s8AAIa-NH₂ | 243 | | 1771.01 | 887.17 | 1772.02 | 886.51 | 591.34 |
| 235 | Ac-LTF$r5AYWAQL$s8SAA-NH₂ | 244 | | 1673.92 | 838.33 | 1674.93 | 837.97 | 558.98 |
| 236 | Ac-LTF$r8AYWAQCba$AANleA-NH₂ | 245 | | 1783.01 | 892.64 | 1784.02 | 892.51 | 595.34 |
| 237 | Ac-ETF$r8AYWAQCba$AANleA-NH₂ | 246 | | 1798.97 | 900.59 | 1799.98 | 900.49 | 600.66 |
| 238 | Ac-LTF$r8EYWAQCba$AANleA-NH₂ | 247 | | 1841.01 | 922.05 | 1842.02 | 921.51 | 614.68 |
| 239 | Ac-LTF$r8AYWAQCba$AWNleA-NH₂ | 248 | | 1898.05 | 950.46 | 1899.06 | 950.03 | 633.69 |
| 240 | Ac-ETF$r8AYWAQCba$AWNleA-NH₂ | 249 | | 1914.01 | 958.11 | 1915.02 | 958.01 | 639.01 |
| 241 | Ac-LTF$r8EYWAQCba$AWNleA-NH₂ | 250 | | 1956.06 | 950.62 | 1957.07 | 979.04 | 653.03 |
| 242 | Ac-LTF$r8EYWAQCba$SAFA-NH₂ | 251 | | 1890.99 | 946.55 | 1892 | 946.5 | 631.34 |
| 243 | Ac-LTF34F2$r8EYWAQCba$SANleA- | 252 | | 1892.99 | 947.57 | 1894 | 947.5 | 632 |
| | NH₂ | | | | | | | |
| 244 | Ac-LTF$r8EF4coohWAQCba$SANleA- | 253 | | 1885 | 943.59 | 1886.01 | 943.51 | 629.34 |
| | NH₂ | | | | | | | |
| 245 | Ac-LTF$r8EYWSQCba$SANleA-NH₂ | 254 | | 1873 | 937.58 | 1874.01 | 937.51 | 625.34 |
| 246 | Ac-LTF$r8EYWWQCba$SANleA-NH₂ | 255 | | 1972.05 | 987.61 | 1973.06 | 987.03 | 658.36 |
| 247 | Ac-LTF$r8EYWAQCba$AAIa-NH₂ | 256 | | 1841.01 | 922.05 | 1842.02 | 921.51 | 614.68 |
| 248 | Ac-LTF34F2$r8EYWAQCba$AAIa-NH₂ | 257 | | 1876.99 | 939.99 | 1878 | 939.5 | 626.67 |
| 249 | Ac-LTF$r8EF4coohWAQCba$AAIa- | 258 | | 1869.01 | 935.64 | 1870.02 | 935.51 | 624.01 |
| | NH₂ | | | | | | | |
| 250 | Pam-ETF$r8EYWAQCba$SAA-NH₂ | 259 | | 1956.1 | 979.57 | 1957.11 | 979.06 | 653.04 |
| 251 | Ac-LThF$r8EFWAQCba$SAA-NH₂ | 260 | | 1741.94 | 872.11 | 1742.95 | 871.98 | 581.65 |
| 252 | Ac-LTA$r8EYWAQCba$SAA-NH₂ | 261 | | 1667.89 | 835.4 | 1668.9 | 834.95 | 556.97 |
| 253 | Ac-LTF$r8EYAAQCba$SAA-NH₂ | 262 | | 1628.88 | 815.61 | 1629.89 | 815.45 | 543.97 |
| 254 | Ac-LTF$r8EY2NalAQCba$SAA-NH₂ | 263 | | 1754.93 | 879.04 | 1755.94 | 878.47 | 585.98 |
| 255 | Ac-LTF$r8AYWAQCba$SAA-NH₂ | 264 | | 1685.92 | 844.71 | 1686.93 | 843.97 | 562.98 |
| 256 | Ac-LTF$r8EYWAQCba$SAF-NH₂ | 265 | | 1819.96 | 911.41 | 1820.97 | 910.99 | 607.66 |
| 257 | Ac-LTF$r8EYWAQCba$SAFa-NH₂ | 266 | | 1890.99 | 947.41 | 1892 | 946.5 | 631.34 |
| 258 | Ac-LTF$r8AYWAQCba$SAF-NH₂ | 267 | | 1761.95 | 882.73 | 1762.96 | 881.98 | 588.32 |
| 259 | Ac-LTF34F2$r8AYWAQCba$SAF-NH₂ | 268 | | 1797.93 | 900.87 | 1798.94 | 899.97 | 600.32 |
| 260 | Ac-LTF$r8AF4coohWAQCba$SAF-NH₂ | 269 | | 1789.94 | 896.43 | 1790.95 | 895.98 | 597.65 |
| 261 | Ac-LTF$r8EY6clWAQCba$SAF-NH₂ | 270 | | 1853.92 | 929.27 | 1854.93 | 927.97 | 618.98 |
| 262 | Ac-LTF$r8AYWSQCba$SAF-NH₂ | 271 | | 1777.94 | 890.87 | 1778.95 | 889.98 | 593.65 |
| 263 | Ac-LTF$r8AYWWQCba$SAF-NH₂ | 272 | | 1876.99 | 939.91 | 1878 | 939.5 | 626.67 |
| 264 | Ac-LTF$r8AYWAQCba$AAIa-NH₂ | 273 | | 1783.01 | 893.19 | 1784.02 | 892.51 | 595.34 |
| 265 | Ac-LTF34F2$r8AYWAQCba$AAIa-NH₂ | 274 | | 1818.99 | 911.23 | 1820 | 910.5 | 607.34 |
| 266 | Ac-LTF$r8AY6clWAQCba$AAIa-NH₂ | 275 | | 1816.97 | 909.84 | 1817.98 | 909.49 | 606.66 |
| 267 | Ac-LTF$r8AF4coohWAQCba$AAIa- | 276 | | 1811 | 906.88 | 1812.01 | 906.51 | 604.67 |
| | NH₂ | | | | | | | |
| 268 | Ac-LTF$r8EYWAQCba$AAFa-NH₂ | 277 | | 1875 | 938.6 | 1876.01 | 938.51 | 626.01 |
| 269 | Ac-LTF$r8EYWAQCba$AAFa-NH₂ | 278 | iso2 | 1875 | 938.6 | 1876.01 | 938.51 | 626.01 |
| 270 | Ac-ETF$r8AYWAQCba$AWNlea-NH₂ | 279 | | 1914.01 | 958.42 | 1915.02 | 958.01 | 639.01 |
| 271 | Ac-LTF$r8EYWAQCba$AWNlea-NH₂ | 280 | | 1956.06 | 979.42 | 1957.07 | 979.04 | 653.03 |
| 272 | Ac-ETF$r8EYWAQCba$AWNlea-NH₂ | 281 | | 1972.01 | 987.06 | 1973.02 | 987.01 | 658.34 |
| 273 | Ac-ETF$r8EYWAQCba$AWNlea-NH₂ | 282 | iso2 | 1972.01 | 987.06 | 1973.02 | 987.01 | 658.34 |
| 274 | Ac-LTF$r8AYWAQCba$SAFa-NH₂ | 283 | | 1832.99 | 917.89 | 1834 | 917.5 | 612 |
| 275 | Ac-LTF$r8AYWAQCba$SAFa-NH₂ | 284 | iso2 | 1832.99 | 918.07 | 1834 | 917.5 | 612 |
| 276 | Ac-ETF$r8AYWAQL$AWNlea-NH₂ | 285 | | 1902.01 | 952.22 | 1903.02 | 952.01 | 635.01 |
| 277 | Ac-LTF$r8EYWAQL$AWNlea-NH₂ | 286 | | 1944.06 | 973.5 | 1945.07 | 973.04 | 649.03 |
| 278 | Ac-ETF$r8EYWAQL$AWNlea-NH₂ | 287 | | 1960.01 | 981.46 | 1961.02 | 981.01 | 654.34 |
| 279 | Dmaac-LTF$r8EYWAQhL$SAA-NH₂ | 288 | | 1788.98 | 896.06 | 1789.99 | 895.5 | 597.33 |
| 280 | Hexac-LTF$r8EYWAQhL$SAA-NH₂ | 289 | | 1802 | 902.9 | 1803.01 | 902.01 | 601.67 |
| 281 | Napac-LTF$r8EYWAQhL$SAA-NH₂ | 290 | | 1871.99 | 937.58 | 1873 | 937 | 625 |
| 282 | Decac-LTF$r8EYWAQhL$SAA-NH₂ | 291 | | 1858.06 | 930.55 | 1859.07 | 930.04 | 620.36 |
| 283 | Admac-LTF$r8EYWAQhL$SAA-NH₂ | 292 | | 1866.03 | 934.07 | 1867.04 | 934.02 | 623.02 |
| 284 | Tmac-LTF$r8EYWAQhL$SAA-NH₂ | 293 | | 1787.99 | 895.41 | 1789 | 895 | 597 |
| 285 | Pam-LTF$r8EYWAQhL$SAA-NH₂ | 294 | | 1942.16 | 972.08 | 1943.17 | 972.09 | 648.39 |
| 286 | Ac-LTF$r8AYWAQCba$AANleA-NH₂ | 295 | iso2 | 1783.01 | 892.64 | 1784.02 | 892.51 | 595.34 |
| 287 | Ac-LTF34F2$r8EYWAQCba$AAIa-NH₂ | 296 | iso2 | 1876.99 | 939.62 | 1878 | 939.5 | 626.67 |
| 288 | Ac-LTF34F2$r8EYWAQCba$SAA-NH₂ | 297 | | 1779.91 | 892.07 | 1780.92 | 890.96 | 594.31 |
| 289 | Ac-LTF34F2$r8EYWAQCba$SAA-NH₂ | 298 | iso2 | 1779.91 | 891.61 | 1780.92 | 890.96 | 594.31 |
| 290 | Ac-LTF$r8EF4coohWAQCba$SAA-NH₂ | 299 | | 1771.92 | 887.54 | 1772.93 | 886.97 | 591.65 |
| 291 | Ac-LTF$r8EF4coohWAQCba$SAA-NH₂ | 300 | iso2 | 1771.92 | 887.63 | 1772.93 | 886.97 | 591.65 |
| 292 | Ac-LTF$r8EYWSQCba$SAA-NH₂ | 301 | | 1759.92 | 881.9 | 1760.93 | 880.97 | 587.65 |
| 293 | Ac-LTF$r8EYWSQCba$SAA-NH₂ | 302 | iso2 | 1759.92 | 881.9 | 1760.93 | 880.97 | 587.65 |
| 294 | Ac-LTF$r8EYWAQhL$SAA-NH₂ | 303 | | 1745.94 | 875.05 | 1746.95 | 873.98 | 582.99 |
| 295 | Ac-LTF$r8AYWAQhL$SAF-NH₂ | 304 | | 1763.97 | 884.02 | 1764.98 | 882.99 | 589 |
| 296 | Ac-LTF$r8AYWAQhL$SAF-NH₂ | 305 | iso2 | 1763.97 | 883.56 | 1764.98 | 882.99 | 589 |
| 297 | Ac-LTF34F2$r8AYWAQhL$SAA-NH₂ | 306 | | 1723.92 | 863.67 | 1724.93 | 862.97 | 575.65 |
| 298 | Ac-LTF34F2$r8AYWAQhL$SAA-NH₂ | 307 | iso2 | 1723.92 | 864.04 | 1724.93 | 862.97 | 575.65 |
| 299 | Ac-LTF$r8AF4coohWAQhL$SAA-NH₂ | 308 | | 1715.93 | 859.44 | 1716.94 | 858.97 | 572.98 |
| 300 | Ac-LTF$r8AF4coohWAQhL$SAA-NH₂ | 309 | iso2 | 1715.93 | 859.6 | 1716.94 | 858.97 | 572.98 |
| 301 | Ac-LTF$r8AYWSQhL$SAA-NH₂ | 310 | | 1703.93 | 853.96 | 1704.94 | 852.97 | 568.98 |
| 302 | Ac-LTF$r8AYWSQhL$SAA-NH₂ | 311 | iso2 | 1703.93 | 853.59 | 1704.94 | 852.97 | 568.98 |
| 303 | Ac-LTF$r8EYWAQL$AANleA-NH₂ | 312 | | 1829.01 | 915.45 | 1830.02 | 915.51 | 610.68 |
| 304 | Ac-LTF34F2$r8AYWAQL$AANleA-NH₂ | 313 | | 1806.99 | 904.58 | 1808 | 904.5 | 603.34 |
| 305 | Ac-LTF$r8AF4coohWAQL$AANleA- | 314 | | 1799 | 901.6 | 1800.01 | 900.51 | 600.67 |
| | NH₂ | | | | | | | |
| 306 | Ac-LTF$r8AYWSQL$AANleA-NH₂ | 315 | | 1787 | 894.75 | 1788.01 | 894.51 | 596.67 |
| 307 | Ac-LTF34F2$r8AYWAQhL$AANleA- | 316 | | 1821 | 911.79 | 1822.01 | 911.51 | 608.01 |
| | NH₂ | | | | | | | |
| 308 | Ac-LTF34F2$r8AYWAQhL$AANleA- | 317 | iso2 | 1821 | 912.61 | 1822.01 | 911.51 | 608.01 |
| | NH₂ | | | | | | | |
| 309 | Ac-LTF$r8AF4coohWAQhL$AANleA- | 318 | | 1813.02 | 907.95 | 1814.03 | 907.52 | 605.35 |
| | NH₂ | | | | | | | |
| 310 | Ac-LTF$r8AF4coohWAQhL$AANleA- | 319 | iso2 | 1813.02 | 908.54 | 1814.03 | 907.52 | 605.35 |
| | NH₂ | | | | | | | |
| 311 | Ac-LTF$r8AYWSQhL$AANleA-NH₂ | 320 | | 1801.02 | 901.84 | 1802.03 | 901.52 | 601.35 |
| 312 | Ac-LTF$r8AYWSQhL$AANleA-NH₂ | 321 | iso2 | 1801.02 | 902.62 | 1802.03 | 901.52 | 601.35 |
| 313 | Ac-LTF$r8AYWAQhL$AAAAa-NH₂ | 322 | | 1814.01 | 908.63 | 1815.02 | 908.01 | 605.68 |
| 314 | Ac-LTF$r8AYWAQhL$AAAAa-NH₂ | 323 | iso2 | 1814.01 | 908.34 | 1815.02 | 908.01 | 605.68 |
| 315 | Ac-LTF$r8AYWAQL$AAAAAa-NH₂ | 324 | | 1871.04 | 936.94 | 1872.05 | 936.53 | 624.69 |
| 316 | Ac-LTF$r8AYWAQL$AAAAAAa-NH₂ | 325 | iso2 | 1942.07 | 972.5 | 1943.08 | 972.04 | 648.37 |
| 317 | Ac-LTF$r8AYWAQL$AAAAAAa-NH₂ | 326 | iso1 | 1942.07 | 972.5 | 1943.08 | 972.04 | 648.37 |
| 318 | Ac-LTF$r8EYWAQhL$AANleA-NH₂ | 327 | | 1843.03 | 922.54 | 1844.04 | 922.52 | 615.35 |
| 319 | Ac-AATF$r8AYWAQL$AANleA-NH₂ | 328 | | 1800 | 901.39 | 1801.01 | 901.01 | 601.01 |
| 320 | Ac-LTF$r8AYWAQL$AANleAA-NH₂ | 329 | | 1842.04 | 922.45 | 1843.05 | 922.03 | 615.02 |
| 321 | Ac-ALTF$r8AYWAQL$AANleAA-NH₂ | 330 | | 1913.08 | 957.94 | 1914.09 | 957.55 | 638.7 |
| 322 | Ac-LTF$r8AYWAQCba$AANleAA-NH₂ | 331 | | 1854.04 | 928.43 | 1855.05 | 928.03 | 619.02 |
| 323 | Ac-LTF$r8AYWAQhL$AANleAA-NH₂ | 332 | | 1856.06 | 929.4 | 1857.07 | 929.04 | 619.69 |
| 324 | Ac-LTF$r8EYWAQCba$SAAA-NH₂ | 333 | | 1814.96 | 909.37 | 1815.97 | 908.49 | 605.99 |
| 325 | Ac-LTF$r8EYWAQCba$SAAA-NH₂ | 334 | iso2 | 1814.96 | 909.37 | 1815.97 | 908.49 | 605.99 |
| 326 | Ac-LTF$r8EYWAQCba$SAAAA-NH₂ | 335 | | 1886 | 944.61 | 1887.01 | 944.01 | 629.67 |
| 327 | Ac-LTF$r8EYWAQCba$SAAAA-NH₂ | 336 | iso2 | 1886 | 944.61 | 1887.01 | 944.01 | 629.67 |
| 328 | Ac-ALTF$r8EYWAQCba$SAA-NH₂ | 337 | | 1814.96 | 909.09 | 1815.97 | 908.49 | 605.99 |
| 329 | Ac-ALTF$r8EYWAQCba$SAAA-NH₂ | 338 | | 1886 | 944.61 | 1887.01 | 944.01 | 629.67 |
| 330 | Ac-ALTF$r8EYWAQCba$SAA-NH₂ | 339 | iso2 | 1814.96 | 909.09 | 1815.97 | 908.49 | 605.99 |
| 331 | Ac-LTF$r8EYWAQL$AAAAAa-NH₂ | 340 | iso2 | 1929.04 | 966.08 | 1930.05 | 965.53 | 644.02 |
| 332 | Ac-LTF$r8EY6clWAQCba$SAA-NH₂ | 341 | | 1777.89 | 890.78 | 1778.9 | 889.95 | 593.64 |
| 333 | Ac- | 342 | | 1918.96 | 961.27 | 1919.97 | 960.49 | 640.66 |
| | LTF$r8EF4cooh6clWAQCba$SANleA- | | | | | | | |
| | NH₂ | | | | | | | |
| 334 | Ac- | 343 | iso2 | 1918.96 | 961.27 | 1919.97 | 960.49 | 640.66 |
| | LTF$r8EF4cooh6clWAQCba$SANleA- | | | | | | | |
| | NH₂ | | | | | | | |
| 335 | Ac- | 344 | | 1902.97 | 953.03 | 1903.98 | 952.49 | 635.33 |
| | LTF$r8EF4cooh6clWAQCba$AAIa- | | | | | | | |
| | NH₂ | | | | | | | |
| 336 | Ac- | 345 | iso2 | 1902.97 | 953.13 | 1903.98 | 952.49 | 635.33 |
| | LTF$r8EF4cooh6clWAQCba$AAIa- | | | | | | | |
| | NH₂ | | | | | | | |
| 337 | Ac-LTF$r8AY6cLWAQL$AAAAAa-NH₂ | 346 | | 1905 | 954.61 | 1906.01 | 953.51 | 636.01 |
| 338 | Ac-LTF$r8AY6clWAQL$AAAAAa-NH₂ | 347 | iso2 | 1905 | 954.9 | 1906.01 | 953.51 | 636.01 |
| 339 | Ac-F$r8AY6clWEAL$AAAAAAa-NH₂ | 348 | | 1762.89 | 883.01 | 1763.9 | 882.45 | 588.64 |
| 340 | Ac-ETF$r8EYWAQL$AAAAAa-NH₂ | 349 | | 1945 | 974.31 | 1946.01 | 973.51 | 649.34 |
| 341 | Ac-ETF$r8EYWAQL$AAAAAa-NH₂ | 350 | iso2 | 1945 | 974.49 | 1946.01 | 973.51 | 649.34 |
| 342 | Ac-LTF$r8EYWAQL$AAAAAAa-NH₂ | 351 | | 2000.08 | 1001.6 | 2001.09 | 1001.05 | 667.7 |
| 343 | Ac-LTF$r8EYWAQL$AAAAAAa-NH₂ | 352 | iso2 | 2000.08 | 1001.6 | 2001.09 | 1001.05 | 667.7 |
| 344 | Ac-LTF$r8AYWAQL$AANleAAa-NH₂ | 353 | | 1913.08 | 958.58 | 1914.09 | 957.55 | 638.7 |
| 345 | Ac-LTF$r8AYWAQL$AANleAAa-NH₂ | 354 | iso2 | 1913.08 | 958.58 | 1914.09 | 957.55 | 638.7 |
| 346 | Ac-LTF$r8EYWAQCba$AAAAAa-NH₂ | 355 | | 1941.04 | 972.55 | 1942.05 | 971.53 | 648.02 |
| 347 | Ac-LTF$r8EYWAQCba$AAAAAa-NH₂ | 356 | iso2 | 1941.04 | 972.55 | 1942.05 | 971.53 | 648.02 |
| 348 | Ac-LTF$r8EF4coohWAQCba$AAAAAa- | 357 | | 1969.04 | 986.33 | 1970.05 | 985.53 | 657.35 |
| | NH₂ | | | | | | | |
| 349 | Ac-LTF$r8EF4coohWAQCba$AAAAAa- | 358 | iso2 | 1969.04 | 986.06 | 1970.05 | 985.53 | 657.35 |
| | NH₂ | | | | | | | |
| 350 | Ac-LTF$r8EYWSQCba$AAAAAa-NH₂ | 359 | | 1957.04 | 980.04 | 1958.05 | 979.53 | 653.35 |
| 351 | Ac-LTF$r8EYWSQCba$AAAAAa-NH₂ | 360 | iso2 | 1957.04 | 980.04 | 1958.05 | 979.53 | 653.35 |
| 352 | Ac-LTF$r8EYWAQCba$SAAa-NH₂ | 361 | | 1814.96 | 909 | 1815.97 | 908.49 | 605.99 |
| 353 | Ac-LTF$r8EYWAQCba$SAAa-NH₂ | 362 | iso2 | 1814.96 | 909 | 1815.97 | 908.49 | 605.99 |
| 354 | Ac-ALTF$r8EYWAQCba$SAAa-NH₂ | 363 | | 1886 | 944.52 | 1887.01 | 944.01 | 629.67 |
| 355 | Ac-ALTF$r8EYWAQCba$SAAa-NH₂ | 364 | iso2 | 1886 | 944.98 | 1887.01 | 944.01 | 629.67 |
| 356 | Ac-ALTF$r8EYWAQCba$SAAAa-NH₂ | 365 | | 1957.04 | 980.04 | 1958.05 | 979.53 | 653.35 |
| 357 | Ac-ALTF$r8EYWAQCba$SAAAa-NH₂ | 366 | iso2 | 1957.04 | 980.04 | 1958.05 | 979.53 | 653.35 |
| 358 | Ac-AALTF$r8EYWAQCba$SAAAa-NH₂ | 367 | | 2028.07 | 1016.1 | 2029.08 | 1015.04 | 677.03 |
| 359 | Ac-AALTF$r8EYWAQCba$SAAAa-NH₂ | 368 | iso2 | 2028.07 | 1015.57 | 2029.08 | 1015.04 | 677.03 |
| 360 | Ac-RTF$r8EYWAQCba$SAA-NH₂ | 369 | | 1786.94 | 895.03 | 1787.95 | 894.48 | 596.65 |
| 361 | Ac-LRF$r8EYWAQCba$SAA-NH₂ | 370 | | 1798.98 | 901.51 | 1799.99 | 900.5 | 600.67 |
| 362 | Ac-LTF$r8EYWRQCba$SAA-NH₂ | 371 | | 1828.99 | 916.4 | 1830 | 915.5 | 610.67 |
| 363 | Ac-LTF$r8EYWARCba$SAA-NH₂ | 372 | | 1771.97 | 887.63 | 1772.98 | 886.99 | 591.66 |
| 364 | Ac-LTF$r8EYWAQCba$RAA-NH₂ | 373 | | 1812.99 | 908.08 | 1814 | 907.5 | 605.34 |
| 365 | Ac-LTF$r8EYWAQCba$SRA-NH₂ | 374 | | 1828.99 | 916.12 | 1830 | 915.5 | 610.67 |
| 366 | Ac-LTF$r8EYWAQCba$SAR-NH₂ | 375 | | 1828.99 | 916.12 | 1830 | 915.5 | 610.67 |
| 367 | 5-FAM-BaLTF$r8EYWAQCba$SAA-NH₂ | 376 | | 2131 | 1067.09 | 2132.01 | 1066.51 | 711.34 |
| 368 | 5-FAM-BaLTF$r8AYWAQL$AANleA- | 377 | | 2158.08 | 1080.6 | 2159.09 | 1080.05 | 720.37 |
| | NH₂ | | | | | | | |
| 369 | Ac-LAF$r8EYWAQL$AANleA-NH₂ | 378 | | 1799 | 901.05 | 1800.01 | 900.51 | 600.67 |
| 370 | Ac-ATF$r8EYWAQL$AANleA-NH₂ | 379 | | 1786.97 | 895.03 | 1787.98 | 894.49 | 596.66 |
| 371 | Ac-AAF$r8EYWAQL$AANleA-NH₂ | 380 | | 1756.96 | 880.05 | 1757.97 | 879.49 | 586.66 |
| 372 | Ac-AAAF$r8EYWAQL$AANleA-NH₂ | 381 | | 1827.99 | 915.57 | 1829 | 915 | 610.34 |
| 373 | Ac-AAAAF$r8EYWAQL$AANleA-NH₂ | 382 | | 1899.03 | 951.09 | 1900.04 | 950.52 | 634.02 |
| 374 | Ac-AATF$r8EYWAQL$AANleA-NH₂ | 383 | | 1858 | 930.92 | 1859.01 | 930.01 | 620.34 |
| 375 | Ac-AALTF$r8EYWAQL$AANleA-NH₂ | 384 | | 1971.09 | 987.17 | 1972.1 | 986.55 | 658.04 |
| 376 | Ac-AAALTF$r8EYWAQL$AANleA-NH₂ | 385 | | 2042.12 | 1023.15 | 2043.13 | 1022.07 | 681.71 |
| 377 | Ac-LTF$r8EYWAQL$AANleAA-NH₂ | 386 | | 1900.05 | 952.02 | 1901.06 | 951.03 | 634.36 |
| 378 | Ac-ALTF$r8EYWAQL$AANleAA-NH₂ | 387 | | 1971.09 | 987.63 | 1972.1 | 986.55 | 658.04 |
| 379 | Ac-AALTF$r8EYWAQL$AANleAA-NH₂ | 388 | | 2042.12 | 1022.69 | 2043.13 | 1022.07 | 681.71 |
| 380 | Ac-LTF$r8EYWAQCba$AANleAA-NH₂ | 389 | | 1912.05 | 958.03 | 1913.06 | 957.03 | 638.36 |
| 381 | Ac-LTF$r8EYWAQhL$AANleAA-NH₂ | 390 | | 1914.07 | 958.68 | 1915.08 | 958.04 | 639.03 |
| 382 | Ac-ALTF$r8EYWAQhL$AANleAA-NH₂ | 391 | | 1985.1 | 994.1 | 1986.11 | 993.56 | 662.71 |
| 383 | Ac-LTF$r8ANmYWAQL$AANleA-NH₂ | 392 | | 1785.02 | 894.11 | 1786.03 | 893.52 | 596.01 |
| 384 | Ac-LTF$r8ANmYWAQL$AANleA-NH₂ | 393 | iso2 | 1785.02 | 894.11 | 1786.03 | 893.52 | 596.01 |
| 385 | Ac-LTF$r8AYNmWAQL$AANleA-NH₂ | 394 | | 1785.02 | 894.11 | 1786.03 | 893.52 | 596.01 |
| 386 | Ac-LTF$r8AYNmWAQL$AANleA-NH₂ | 395 | iso2 | 1785.02 | 894.11 | 1786.03 | 893.52 | 596.01 |
| 387 | Ac-LTF$r8AYAmwAQL$AANleA-NH₂ | 396 | | 1785.02 | 894.01 | 1786.03 | 893.52 | 596.01 |
| 388 | Ac-LTF$r8AYAmwAQL$AANleA-NH₂ | 397 | iso2 | 1785.02 | 894.01 | 1786.03 | 893.52 | 596.01 |
| 389 | Ac-LTF$r8AYWAibQL$AANleA-NH₂ | 398 | | 1785.02 | 894.01 | 1786.03 | 893.52 | 596.01 |
| 390 | Ac-LTF$r8AYWAibQL$AANleA-NH₂ | 399 | iso2 | 1785.02 | 894.01 | 1786.03 | 893.52 | 596.01 |
| 391 | Ac-LTF$r8AYWAQL$AAibNleA-NH₂ | 400 | | 1785.02 | 894.38 | 1786.03 | 893.52 | 596.01 |
| 392 | Ac-LTF$r8AYWAQL$AAibNleA-NH₂ | 401 | iso2 | 1785.02 | 894.38 | 1786.03 | 893.52 | 596.01 |
| 393 | Ac-LTF$r8AYWAQL$AaNleA-NH₂ | 402 | | 1771.01 | 887.54 | 1772.02 | 886.51 | 591.34 |
| 394 | Ac-LTF$r8AYWAQL$AaNleA-NH₂ | 403 | iso2 | 1771.01 | 887.54 | 1772.02 | 886.51 | 591.34 |
| 395 | Ac-LTF$r8AYWAQL$ASarNleA-NH₂ | 404 | | 1771.01 | 887.35 | 1772.02 | 886.51 | 591.34 |
| 396 | Ac-LTF$r8AYWAQL$ASarNleA-NH₂ | 405 | iso2 | 1771.01 | 887.35 | 1772.02 | 886.51 | 591.34 |
| 397 | Ac-LTF$r8AYWAQL$AANleAib-NH₂ | 406 | | 1785.02 | 894.75 | 1786.03 | 893.52 | 596.01 |
| 398 | Ac-LTF$r8AYWAQL$AANleAib-NH₂ | 407 | iso2 | 1785.02 | 894.75 | 1786.03 | 893.52 | 596.01 |
| 399 | Ac-LTF$r8AYWAQL$AANleNmA-NH₂ | 408 | | 1785.02 | 894.6 | 1786.03 | 893.52 | 596.01 |
| 400 | Ac-LTF$r8AYWAQL$AANleNmA-NH₂ | 409 | iso2 | 1785.02 | 894.6 | 1786.03 | 893.52 | 596.01 |
| 401 | Ac-LTF$r8AYWAQL$AANleSar-NH₂ | 410 | | 1771.01 | 886.98 | 1772.02 | 886.51 | 591.34 |
| 402 | Ac-LTF$r8AYWAQL$AANleSar-NH₂ | 411 | iso2 | 1771.01 | 886.98 | 1772.02 | 886.51 | 591.34 |
| 403 | Ac-LTF$r8AYWAQL$AANleAAib-NH₂ | 412 | | 1856.06 | | 1857.07 | 929.04 | 619.69 |
| 404 | Ac-LTF$r8AYWAQL$AANleAAib-NH₂ | 413 | iso2 | 1856.06 | | 1857.07 | 929.04 | 619.69 |
| 405 | Ac-LTF$r8AYWAQL$AANleANmA-NH₂ | 414 | | 1856.06 | 930.37 | 1857.07 | 929.04 | 619.69 |
| 406 | Ac-LTF$r8AYWAQL$AANleANmA-NH₂ | 415 | iso2 | 1856.06 | 930.37 | 1857.07 | 929.04 | 619.69 |
| 407 | Ac-LTF$r8AYWAQL$AANleAa-NH₂ | 416 | | 1842.04 | 922.69 | 1843.05 | 922.03 | 615.02 |
| 408 | Ac-LTF$r8AYWAQL$AANleAa-NH₂ | 417 | iso2 | 1842.04 | 922.69 | 1843.05 | 922.03 | 615.02 |
| 409 | Ac-LTF$r8AYWAQL$AANleASar-NH₂ | 418 | | 1842.04 | 922.6 | 1843.05 | 922.03 | 615.02 |
| 410 | Ac-LTF$r8AYWAQL$AANleASar-NH₂ | 419 | iso2 | 1842.04 | 922.6 | 1843.05 | 922.03 | 615.02 |
| 411 | Ac-LTF$/r8AYWAQL$/AANleA-NH₂ | 420 | | 1799.04 | 901.14 | 1800.05 | 900.53 | 600.69 |
| 412 | Ac-LTFAibAYWAQLAibAANleA-NH₂ | 421 | | 1648.9 | 826.02 | 1649.91 | 825.46 | 550.64 |
| 413 | Ac-LTF$r8Cou4YWAQL$AANleA-NH₂ | 422 | | 1975.05 | 989.11 | 1976.06 | 988.53 | 659.36 |
| 414 | Ac-LTF$r8Cou4YWAQL$AANleA-NH₂ | 423 | iso2 | 1975.05 | 989.11 | 1976.06 | 988.53 | 659.36 |
| 415 | Ac-LTF$r8AYWCou4QL$AANleA-NH₂ | 424 | | 1975.05 | 989.11 | 1976.06 | 988.53 | 659.36 |
| 416 | Ac-LTF$r8AYWAQL$Cou4ANleA-NH₂ | 425 | | 1975.05 | 989.57 | 1976.06 | 988.53 | 659.36 |
| 417 | Ac-LTF$r8AYWAQL$Cou4ANleA-NH₂ | 426 | iso2 | 1975.05 | 989.57 | 1976.06 | 988.53 | 659.36 |
| 418 | Ac-LTF$r8AYWAQL$ACou4NleA-NH₂ | 427 | | 1975.05 | 989.57 | 1976.06 | 988.53 | 659.36 |
| 419 | Ac-LTF$r8AYWAQL$ACou4NleA-NH₂ | 428 | iso2 | 1975.05 | 989.57 | 1976.06 | 988.53 | 659.36 |
| 420 | Ac-LTF$r8AYWAQL$AANleA-OH | 429 | | 1771.99 | 887.63 | 1773 | 887 | 591.67 |
| 421 | Ac-LTF$r8AYWAQL$AANleA-OH | 430 | iso2 | 1771.99 | 887.63 | 1773 | 887 | 591.67 |
| 422 | Ac-LTF$r8AYWAQL$AANleA-NHnPr | 431 | | 1813.05 | 908.08 | 1814.06 | 907.53 | 605.36 |
| 423 | Ac-LTF$r8AYWAQL$AANleA-NHnPr | 432 | iso2 | 1813.05 | 908.08 | 1814.06 | 907.53 | 605.36 |
| 424 | Ac-LTF$r8AYWAQL$AANleA- | 433 | | 1855.1 | 929.17 | 1856.11 | 928.56 | 619.37 |
| | NHnBu33me | | | | | | | |
| 425 | Ac-LTF$r8AYWAQL$AANleA- | 434 | iso2 | 1855.1 | 929.17 | 1856.11 | 928.56 | 619.37 |
| | NHnBu33Me | | | | | | | |
| 426 | Ac-LTF$r8AYWAQL$AANleA-NHHex | 435 | | 1855.1 | 929.17 | 1856.11 | 928.56 | 619.37 |
| 427 | Ac-LTF$r8AYWAQL$AANleA-NHHex | 436 | iso2 | 1855.1 | 929.17 | 1856.11 | 928.56 | 619.37 |
| 428 | Ac-LTA$r8AYWAQL$AANleA-NH₂ | 437 | | 1694.98 | 849.33 | 1695.99 | 848.5 | 566 |
| 429 | Ac-LThL$r8AYWAQL$AANleA-NH₂ | 438 | | 1751.04 | 877.09 | 1752.05 | 876.53 | 584.69 |
| 430 | Ac-LTF$r8AYAAQL$AANleA-NH₂ | 439 | | 1655.97 | 829.54 | 1656.98 | 828.99 | 553 |
| 431 | Ac-LTF$r8AY2NalAQL$AANleA-NH₂ | 440 | | 1782.01 | 892.63 | 1783.02 | 892.01 | 595.01 |
| 432 | Ac-LTF$r8EYWCou4QCba$SAA-NH₂ | 441 | | 1947.97 | 975.8 | 1948.98 | 974.99 | 650.33 |
| 433 | Ac-LTF$r8EYWCou7QCba$SAA-NH₂ | 442 | | 16.03 | 974.9 | 17.04 | 9.02 | 6.35 |
| 434 | Ac-LTF%r8EYWAQCba%SAA-NH₂ | 443 | | 1745.94 | 874.8 | 1746.95 | 873.98 | 582.99 |
| 435 | Dmaac-LTF$r8EYWAQCba$SAA-NH₂ | 444 | | 1786.97 | 894.8 | 1787.98 | 894.49 | 596.66 |
| 436 | Dmaac-LTF$r8AYWAQL$AAAAAa-NH₂ | 445 | | 1914.08 | 958.2 | 1915.09 | 958.05 | 639.03 |
| 437 | Dmaac-LTF$r8AYWAQL$AAAAAa-NH₂ | 446 | iso2 | 1914.08 | 958.2 | 1915.09 | 958.05 | 639.03 |
| 438 | Dmaac-LTF$r8EYWAQL$AAAAAa-NH₂ | 447 | | 1972.08 | 987.3 | 1973.09 | 987.05 | 658.37 |
| 439 | Dmaac-LTF$r8EYWAQL$AAAAAa-NH₂ | 448 | iso2 | 1972.08 | 987.3 | 1973.09 | 987.05 | 658.37 |
| 440 | Dmaac- | 449 | | 1912.05 | 957.4 | 1913.06 | 957.03 | 638.36 |
| | LTF$r8EF4coohWAQCba$AAIa-NH₂ | | | | | | | |
| 441 | Dmaac- | 450 | iso2 | 1912.05 | 957.4 | 1913.06 | 957.03 | 638.36 |
| | LTF$r8EF4coohWAQCba$AAIa-NH₂ | | | | | | | |
| 442 | Dmaac-LTF$r8AYWAQL$AANleA-NH₂ | 451 | | 1814.05 | 908.3 | 1815.06 | 908.03 | 605.69 |
| 443 | Dmaac-LTF$r8AYWAQL$AANleA-NH₂ | 452 | iso2 | 1814.05 | 908.3 | 1815.06 | 908.03 | 605.69 |
| 444 | Ac-LTF%r8AYWAQL%AANleA-NH₂ | 453 | | 1773.02 | 888.37 | 1774.03 | 887.52 | 592.01 |
| 445 | Ac-LTF%r8EYWAQL%AAAAAa-NH₂ | 454 | | 1931.06 | 966.4 | 1932.07 | 966.54 | 644.69 |
| 446 | Cou6BaLTF$r8EYWAQhL$SAA-NH₂ | 455 | | 2018.05 | 1009.9 | 2019.06 | 1010.03 | 673.69 |
| 447 | Cou8BaLTF$r8EYWAQhL$SAA-NH₂ | 456 | | 1962.96 | 982.34 | 1963.97 | 982.49 | 655.32 |
| 448 | Ac-LTF4I$r8EYWAQL$AAAAAa-NH₂ | 457 | | 2054.93 | 1028.68 | 2055.94 | 1028.47 | 685.98 |
| 449 | Ac-LTF$r8EYWAQL$AAAAAa-NH₂ | 458 | | 1929.04 | 966.17 | 1930.05 | 965.53 | 644.02 |
| 550 | Ac-LTF$r8EYWAQL$AAAAAa-OH | 459 | | 1930.02 | 966.54 | 1931.03 | 966.02 | 644.35 |
| 551 | Ac-LTF$r8EYWAQL$AAAAAa-OH | 460 | iso2 | 1930.02 | 965.89 | 1931.03 | 966.02 | 644.35 |
| 552 | Ac-LTF$r8EYwAEL$AAAAAa-NH₂ | 461 | | 1930.02 | 966.82 | 1931.03 | 966.02 | 644.35 |
| 553 | Ac-LTF$r8EYWAEL$AAAAAa-NH₂ | 462 | iso2 | 1930.02 | 966.91 | 1931.03 | 966.02 | 644.35 |
| 554 | Ac-LTF$r8EYWAEL$AAAAAa-OH | 463 | | 1931.01 | 967.28 | 1932.02 | 966.51 | 644.68 |
| 555 | Ac-LTF$r8EY6clWAQL$AAAAAa-NH₂ | 464 | | 1963 | 983.28 | 1964.01 | 982.51 | 655.34 |
| 556 | Ac-LTF$r8EF4bOH2wAQL$AAAAAa- | 465 | | 1957.05 | 980.04 | 1958.06 | 979.53 | 653.36 |
| | NH₂ | | | | | | | |
| 557 | Ac-AAALTF$r8EYWAQL$AAAAAa-NH₂ | 466 | | 2142.15 | 1072.83 | 2143.16 | 1072.08 | 715.06 |
| 558 | Ac-LTF34F2$r8EYWAQL$AAAAAa-NH₂ | 467 | | 1965.02 | 984.3 | 1966.03 | 983.52 | 656.01 |
| 559 | Ac-RTF$r8EYWAQL$AAAAAa-NH₂ | 468 | | 1972.06 | 987.81 | 1973.07 | 987.04 | 658.36 |
| 560 | Ac-LTA$r8EYWAQL$AAAAAa-NH₂ | 469 | | 1853.01 | 928.33 | 1854.02 | 927.51 | 618.68 |
| 561 | Ac-LTF$r8EYWAibQL$AAAAAa-NH₂ | 470 | | 1943.06 | 973.48 | 1944.07 | 972.54 | 648.69 |
| 562 | Ac-LTF$r8EYWAQL$AAibAAAa-NH₂ | 471 | | 1943.06 | 973.11 | 1944.07 | 972.54 | 648.69 |
| 563 | Ac-LTF$r8EYWAQL$AAAibAAa-NH₂ | 472 | | 1943.06 | 973.48 | 1944.07 | 972.54 | 648.69 |
| 564 | Ac-LTF$r8EYWAQL$AAAAibAa-NH₂ | 473 | | 1943.06 | 973.48 | 1944.07 | 972.54 | 648.69 |
| 565 | Ac-LTF$r8EYWAQL$AAAAAiba-NH₂ | 474 | | 1943.06 | 973.38 | 1944.07 | 972.54 | 648.69 |
| 566 | Ac-LTF$r8EYWAQL$AAAAAiba-NH₂ | 475 | iso2 | 1943.06 | 973.38 | 1944.07 | 972.54 | 648.69 |
| 567 | Ac-LTF$r8EYWAQL$AAAAAAib-NH₂ | 476 | | 1943.06 | 973.01 | 1944.07 | 972.54 | 648.69 |
| 568 | Ac-LTF$r8EYWAQL$AaAAAa-NH₂ | 477 | | 1929.04 | 966.54 | 1930.05 | 965.53 | 644.02 |
| 569 | Ac-LTF$r8EYWAQL$AAaAAa-NH₂ | 478 | | 1929.04 | 966.35 | 1930.05 | 965.53 | 644.02 |
| 570 | Ac-LTF$r8EYWAQL$AAAaAa-NH₂ | 479 | | 1929.04 | 966.54 | 1930.05 | 965.53 | 644.02 |
| 571 | Ac-LTF$r8EYWAQL$AAAaAa-NH₂ | 480 | iso2 | 1929.04 | 966.35 | 1930.05 | 965.53 | 644.02 |
| 572 | Ac-LTF$r8EYWAQL$AAAAaa-NH₂ | 481 | | 1929.04 | 966.35 | 1930.05 | 965.53 | 644.02 |
| 573 | Ac-LTF$r8EYWAQL$AAAAAA-NH₂ | 482 | | 1929.04 | 966.35 | 1930.05 | 965.53 | 644.02 |
| 574 | Ac-LTF$r8EYWAQL$ASarAAAa-NH₂ | 483 | | 1929.04 | 966.54 | 1930.05 | 965.53 | 644.02 |
| 575 | Ac-LTF$r8EYWAQL$AASarAAa-NH₂ | 484 | | 1929.04 | 966.35 | 1930.05 | 965.53 | 644.02 |
| 576 | Ac-LTF$r8EYWAQL$AAASarAa-NH₂ | 485 | | 1929.04 | 966.35 | 1930.05 | 965.53 | 644.02 |
| 577 | Ac-LTF$r8EYWAQL$AAAASara-NH₂ | 486 | | 1929.04 | 966.35 | 1930.05 | 965.53 | 644.02 |
| 578 | Ac-LTF$r8EYWAQL$AAAAASar-NH₂ | 487 | | 1929.04 | 966.08 | 1930.05 | 965.53 | 644.02 |
| 579 | Ac-7LTF$r8EYWAQL$AAAAAa-NH₂ | 488 | | 1918.07 | 951.99 | 1919.08 | 960.04 | 640.37 |
| 581 | Ac-TF$r8EYWAQL$AAAAAa-NH₂ | 489 | | 1815.96 | 929.85 | 1816.97 | 908.99 | 606.33 |
| 582 | Ac-F$r8EYWAQL$AAAAAa-NH₂ | 490 | | 1714.91 | 930.92 | 1715.92 | 858.46 | 572.64 |
| 583 | Ac-LVF$r8EYWAQL$AAAAAa-NH₂ | 491 | | 1927.06 | 895.12 | 1928.07 | 964.54 | 643.36 |
| 584 | Ac-AAF$r8EYWAQL$AAAAAa-NH₂ | 492 | | 1856.98 | 859.51 | 1857.99 | 929.5 | 620 |
| 585 | Ac-LTF$r8EYWAQL$AAAAa-NH₂ | 493 | | 1858 | 824.08 | 1859.01 | 930.01 | 620.34 |
| 586 | Ac-LTF$r8EYWAQL$AAAa-NH₂ | 494 | | 1786.97 | 788.56 | 1787.98 | 894.49 | 596.66 |
| 587 | Ac-LTF$r8EYWAQL$AAa-NH₂ | 495 | | 1715.93 | 1138.57 | 1716.94 | 858.97 | 572.98 |
| 588 | Ac-LTF$r8EYWAQL$Aa-NH₂ | 496 | | 1644.89 | 1144.98 | 1645.9 | 823.45 | 549.3 |
| 589 | Ac-LTF$r8EYWAQL$a-NH₂ | 497 | | 1573.85 | 1113.71 | 1574.86 | 787.93 | 525.62 |
| 590 | Ac-LTF$r8EYWAQL$AAA-OH | 498 | | 1716.91 | 859.55 | 1717.92 | 859.46 | 573.31 |
| 591 | Ac-LTF$r8EYWAQL$A-OH | 499 | | 1574.84 | 975.14 | 1575.85 | 788.43 | 525.95 |
| 592 | Ac-LTF$r8EYWAQL$AAA-NH₂ | 500 | | 1715.93 | 904.75 | 1716.94 | 858.97 | 572.98 |
| 593 | Ac-LTF$r8EYWAQCba$SAA-OH | 501 | | 1744.91 | 802.49 | 1745.92 | 873.46 | 582.64 |
| 594 | Ac-LTF$r8EYWAQCba$S-OH | 502 | | 1602.83 | 913.53 | 1603.84 | 802.42 | 535.28 |
| 595 | Ac-LTF$r8EYWAQCba$S-NH₂ | 503 | | 1601.85 | 979.58 | 1602.86 | 801.93 | 534.96 |
| 596 | 4-FBzl-LTF$r8EYWAQL$AAAAAa-NH₂ | 504 | | 2009.05 | 970.52 | 2010.06 | 1005.53 | 670.69 |
| 597 | 4-FBzl-LTF$r8EYWAQCba$SAA-NH₂ | 505 | | 1823.93 | 965.8 | 1824.94 | 912.97 | 608.98 |
| 598 | Ac-LTF$r8RYWAQL$AAAAAa-NH₂ | 506 | | 1956.1 | 988.28 | 1957.11 | 979.06 | 653.04 |
| 599 | Ac-LTF$r8HYWAQL$AAAAAa-NH₂ | 507 | | 1937.06 | 1003.54 | 1938.07 | 969.54 | 646.69 |
| 600 | Ac-LTF$r8QYWAQL$AAAAAa-NH₂ | 508 | | 1928.06 | 993.92 | 1929.07 | 965.04 | 643.69 |
| 601 | Ac-LTF$r8CitYWAQL$AAAAAa-NH₂ | 509 | | 1957.08 | 987 | 1958.09 | 979.55 | 653.37 |
| 602 | Ac-LTF$r8GlaYWAQL$AAAAAa-NH₂ | 510 | | 1973.03 | 983 | 1974.04 | 987.52 | 658.68 |
| 603 | Ac-LTF$r8F4gYWAQL$AAAAAa-NH₂ | 511 | | 2004.1 | 937.86 | 2005.11 | 1003.06 | 669.04 |
| 604 | Ac-LTF$r82mRYWAQL$AAAAAa-NH₂ | 512 | | 1984.13 | 958.58 | 1985.14 | 993.07 | 662.38 |
| 605 | Ac-LTF$r8ipKYWAQL$AAAAAa-NH₂ | 513 | | 1970.14 | 944.52 | 1971.15 | 986.08 | 657.72 |
| 606 | Ac-LTF$r8F4NH2YWAQL$AAAAAa-NH₂ | 514 | | 1962.08 | 946 | 1963.09 | 982.05 | 655.03 |
| 607 | Ac-LTF$r8EYWAAL$AAAAAa-NH₂ | 515 | | 1872.02 | 959.32 | 1873.03 | 937.02 | 625.01 |
| 608 | Ac-LTF$r8EYWALL$AAAAAa-NH₂ | 516 | | 1914.07 | 980.88 | 1915.08 | 958.04 | 639.03 |
| 609 | Ac-LTF$r8EYWAAibL$AAAAAa-NH₂ | 517 | | 1886.03 | 970.61 | 1887.04 | 944.02 | 629.68 |
| 610 | Ac-LTF$r8EYWASL$AAAAAa-NH₂ | 518 | | 1888.01 | 980.51 | 1889.02 | 945.01 | 630.34 |
| 611 | Ac-LTF$r8EYWANL$AAAAAa-NH₂ | 519 | | 1915.02 | 1006.41 | 1916.03 | 958.52 | 639.35 |
| 612 | Ac-LTF$r8EYWACitL$AAAAAa-NH₂ | 520 | | 1958.07 | | 1959.08 | 980.04 | 653.7 |
| 613 | Ac-LTF$r8EYWAHL$AAAAAa-NH₂ | 521 | | 1938.04 | 966.24 | 1939.05 | 970.03 | 647.02 |
| 614 | Ac-LTF$r8EYWARL$AAAAAa-NH₂ | 522 | | 1957.08 | | 1958.09 | 979.55 | 653.37 |
| 615 | Ac-LTF$r8EpYWAQL$AAAAAa-NH₂ | 523 | | 2009.01 | | 2010.02 | 1005.51 | 670.68 |
| 616 | Cbm-LTF$r8EYWAQCba$SAA-NH₂ | 524 | | 1590.85 | | 1591.86 | 796.43 | 531.29 |
| 617 | Cbm-LTF$r8EYWAQL$AAAAAa-NH₂ | 525 | | 1930.04 | | 1931.05 | 966.03 | 644.35 |
| 618 | Ac-LTF$r8EYWAQL$SAAAAa-NH₂ | 526 | | 1945.04 | 1005.11 | 1946.05 | 973.53 | 649.35 |
| 619 | Ac-LTF$r8EYWAQL$AAAASa-NH₂ | 527 | | 1945.04 | 986.52 | 1946.05 | 973.53 | 649.35 |
| 620 | Ac-LTF$r8EYWAQL$SAAASa-NH₂ | 528 | | 1961.03 | 993.27 | 1962.04 | 981.52 | 654.68 |
| 621 | Ac-LTF$r8EYWAQTba$AAAAAa-NH₂ | 529 | | 1943.06 | 983.1 | 1944.07 | 972.54 | 648.69 |
| 622 | Ac-LTF$r8EYWAQAdm$AAAAAa-NH₂ | 530 | | 2007.09 | 990.31 | 2008.1 | 1004.55 | 670.04 |
| 623 | Ac-LTF$r8EYWAQCha$AAAAAa-NH₂ | 531 | | 1969.07 | 987.17 | 1970.08 | 985.54 | 657.36 |
| 624 | Ac-LTF$r8EYWAQhCha$AAAAAa-NH₂ | 532 | | 1983.09 | 1026.11 | 1984.1 | 992.55 | 662.04 |
| 625 | Ac-LTF$r8EYWAQF$AAAAAa-NH₂ | 533 | | 1963.02 | 957.01 | 1964.03 | 982.52 | 655.35 |
| 626 | Ac-LTF$r8EYWAQhF$AAAAAa-NH₂ | 534 | | 1977.04 | 1087.81 | 1978.05 | 989.53 | 660.02 |
| 627 | Ac-LTF$r8EYWAQL$AANleAAa-NH₂ | 535 | | 1971.09 | 933.45 | 1972.1 | 986.55 | 658.04 |
| 628 | Ac-LTF$r8EYWAQAdm$AANleAAa-NH₂ | 536 | | 2049.13 | 1017.97 | 2050.14 | 1025.57 | 684.05 |
| 629 | 4-FBz-BaLTF$r8EYWAQL$AAAAAa- | 537 | | 2080.08 | | 2081.09 | 1041.05 | 694.37 |
| | NH₂ | | | | | | | |
| 630 | 4-FBz-BaLTF$r8EYWAQCba$SAA-NH₂ | 538 | | 1894.97 | | 1895.98 | 948.49 | 632.66 |
| 631 | Ac-LTF$r5EYWAQL$s8AAAAAa-NH₂ | 539 | | 1929.04 | 1072.68 | 1930.05 | 965.53 | 644.02 |
| 632 | Ac-LTF$r5EYWAQCba$s8SAA-NH₂ | 540 | | 1743.92 | 1107.79 | 1744.93 | 872.97 | 582.31 |
| 633 | Ac-LTF$r8EYWAQL$AAhhLAAa-NH₂ | 541 | | 1999.12 | | 2000.13 | 1000.57 | 667.38 |
| 634 | Ac-LTF$r8EYWAQL$AAAAAAAa-NH₂ | 542 | | 2071.11 | | 2072.12 | 1036.56 | 691.38 |
| 635 | Ac-LTF$r8EYWAQL$AAAAAAAAa-NH₂ | 543 | | 2142.15 | 778.1 | 2143.16 | 1072.08 | 715.06 |
| 636 | Ac-LTF$r8EYWAQL$AAAAAAAAAa-NH₂ | 544 | | 2213.19 | 870.53 | 2214.2 | 1107.6 | 738.74 |
| 637 | Ac-LTA$r8EYAAQCba$SAA-NH₂ | 545 | | 1552.85 | | 1553.86 | 777.43 | 518.62 |
| 638 | Ac-LTA$r8EYAAQL$AAAAAa-NH₂ | 546 | | 1737.97 | 779.45 | 1738.98 | 869.99 | 580.33 |
| 639 | Ac-LTF$r8EPmpWAQL$AAAAAa-NH₂ | 547 | | 2007.03 | 779.54 | 2008.04 | 1004.52 | 670.02 |
| 640 | Ac-LTF$r8EPmpWAQCba$SAA-NH₂ | 548 | | 1821.91 | 838.04 | 1822.92 | 911.96 | 608.31 |
| 641 | Ac-ATF$r8HYWAQL$S-NH₂ | 549 | | 1555.82 | 867.83 | 1556.83 | 778.92 | 519.61 |
| 642 | Ac-LTF$r8HAWAQL$S-NH₂ | 550 | | 1505.84 | 877.91 | 1506.85 | 753.93 | 502.95 |
| 643 | Ac-LTF$r8HYWAQA$S-NH₂ | 551 | | 1555.82 | 852.52 | 1556.83 | 778.92 | 519.61 |
| 644 | Ac-LTF$r8EYWAQCba$SA-NH₂ | 552 | | 1672.89 | 887.18 | 1673.9 | 837.45 | 558.64 |
| 645 | Ac-LTF$r8EYWAQL$SAA-NH₂ | 553 | | 1731.92 | 873.32 | 1732.93 | 866.97 | 578.31 |
| 646 | Ac-LTF$r8HYWAQCba$SAA-NH₂ | 554 | | 1751.94 | 873.05 | 1752.95 | 876.98 | 584.99 |
| 647 | Ac-LTF$r8SYWAQCba$SAA-NH₂ | 555 | | 1701.91 | 844.88 | 1702.92 | 851.96 | 568.31 |
| 648 | Ac-LTF$r8RYWAQCba$SAA-NH₂ | 556 | | 1770.98 | 865.58 | 1771.99 | 886.5 | 591.33 |
| 649 | Ac-LTF$r8KYWAQCba$SAA-NH₂ | 557 | | 1742.98 | 936.57 | 1743.99 | 872.5 | 582 |
| 650 | Ac-LTF$r8QYWAQCba$SAA-NH₂ | 558 | | 1742.94 | 930.93 | 1743.95 | 872.48 | 581.99 |
| 651 | Ac-LTF$r8EYWAACba$SAA-NH₂ | 559 | | 1686.9 | 1032.45 | 1687.91 | 844.46 | 563.31 |
| 652 | Ac-LTF$r8EYWAQCba$AAA-NH₂ | 560 | | 1727.93 | 895.46 | 1728.94 | 864.97 | 576.98 |
| 653 | Ac-LTF$r8EYWAQL$AAAAA-OH | 561 | | 1858.99 | 824.54 | 1860 | 930.5 | 620.67 |
| 654 | Ac-LTF$r8EYWAQL$AAAA-OH | 562 | | 1787.95 | 894.48 | 1788.96 | 894.98 | 596.99 |
| 655 | Ac-LTF$r8EYWAQL$AA-OH | 563 | | 1645.88 | 856 | 1646.89 | 823.95 | 549.63 |
| 656 | Ac-LTF$r8AF4bOH2WAQL$AAAAAa- | 564 | | | | | | |
| | NH₂ | | | | | | | |
| 657 | Ac-LTF$r8AF4bOH2WAAL$AAAAAa- | 565 | | | | | | |
| | NH₂ | | | | | | | |
| 658 | Ac-LTF$r8EF4bOH2WAQCba$SAA-NH₂ | 566 | | | | | | |
| 659 | Ac-LTF$r8ApYWAQL$AAAAAa-NH₂ | 567 | | | | | | |
| 660 | Ac-LTF$r8ApYWAAL$AAAAAa-NH₂ | 568 | | | | | | |
| 661 | Ac-LTF$r8EpYWAQCba$SAA-NH₂ | 569 | | | | | | |
| 662 | Ac-LTF$rda6AYWAQL$da5AAAAAa- | 570 | | 1974.06 | 934.44 | | | |
| | NH₂ | | | | | | | |
| 663 | Ac-LTF$rda6EYWAQCba$da5SAA-NH₂ | 571 | | 1846.95 | 870.52 | | 869.94 | |
| 664 | Ac-LTF$rda6EYWAQL$da5AAAAAa- | 572 | | | | | | |
| | NH₂ | | | | | | | |
| 665 | Ac-LTF$ra9EYWAQL$a6AAAAAa-NH₂ | 573 | | 936.57 | | 935.51 | | |
| 666 | Ac-LTF$ra9EYWAQL$a6AAAAAa-NH₂ | 574 | | | | | | |
| 667 | Ac-LTF$ra9EYWAQCba$a6SAA-NH₂ | 575 | | | | | | |
| 668 | Ac-LTA$ra9EYWAQCba$a6SAA-NH₂ | 576 | | | | | | |
| 669 | 5-FAM-BaLTF$ra9EYWAQCba$a6SAA- | 577 | | | | | | |
| | NH₂ | | | | | | | |
| 670 | 5-FAM-BaLTF$r8EYWAQL$AAAAAa- | 578 | | 2316.11 | | | | |
| | NH₂ | | | | | | | |
| 671 | 5-FAM-BaLTF$/r8EYWAQL$/AAAAAa- | 579 | | 2344.15 | | | | |
| | NH₂ | | | | | | | |
| 672 | 5-FAM-BaLTA$r8EYWAQL$AAAAAa- | 580 | | 2240.08 | | | | |
| | NH₂ | | | | | | | |
| 673 | 5-FAM-BaLTF$r8AYWAQL$AAAAAa- | 581 | | 2258.11 | | | | |
| | NH₂ | | | | | | | |
| 674 | 5-FAM-BaATF$r8EYWAQL$AAAAAa- | 582 | | 2274.07 | | | | |
| | NH₂ | | | | | | | |
| 675 | 5-FAM-BaLAF$r8EYWAQL$AAAAAa- | 583 | | 2286.1 | | | | |
| | NH₂ | | | | | | | |
| 676 | 5-FAM-BaLTF$r8EAWAQL$AAAAAa- | 584 | | 2224.09 | | | | |
| | NH₂ | | | | | | | |
| 677 | 5-FAM-BaLTF$r8EYAAQL$AAAAAa- | 585 | | 2201.07 | | | | |
| | NH₂ | | | | | | | |
| 678 | 5-FAM-BaLTA$r8EYAAQL$AAAAAa- | 586 | | 2125.04 | | | | |
| | NH₂ | | | | | | | |
| 679 | 5-FAM-BaLTF$r8EYWAAL$AAAAAa- | 587 | | 2259.09 | | | | |
| | NH₂ | | | | | | | |
| 680 | 5-FAM-BaLTF$r8EYWAQA$AAAAAa- | 588 | | 2274.07 | | | | |
| | NH₂ | | | | | | | |
| 681 | 5-FAM-BaLTF$/r8EYWAQCba$/SAA- | 589 | | 2159.03 | | | | |
| | NH₂ | | | | | | | |
| 682 | 5-FAM-BaLTA$r8EYWAQCba$SAA-NH₂ | 590 | | 2054.97 | | | | |
| 683 | 5-FAM-BaLTF$r8EYAAQCba$SAA-NH₂ | 591 | | 2015.96 | | | | |
| 684 | 5-FAM-BaLTA$r8EYAAQCba$SAA-NH₂ | 592 | | 1939.92 | | | | |
| 685 | 5-FAM-BaQSQQTF$r8NLWRLL$QN-NH₂ | 593 | | 2495.23 | | | | |
| 686 | 5-TAMRA-BaLTF$r8EYWAQCba$SAA- | 594 | | 2186.1 | | | | |
| | NH₂ | | | | | | | |
| 687 | 5-TAMRA-BaLTA$r8EYWAQCba$SAA- | 595 | | 2110.07 | | | | |
| | NH₂ | | | | | | | |
| 688 | 5-TAMRA-BaLTF$r8EYAAQCba$SAA- | 596 | | 2071.06 | | | | |
| | NH₂ | | | | | | | |
| 689 | 5-TAMRA-BaLTA$r8EYAAQCba$SAA- | 597 | | 1995.03 | | | | |
| | NH₂ | | | | | | | |
| 690 | 5-TAMRA- | 598 | | 2214.13 | | | | |
| | BaLTF$/r8EYWAQCba$/SAA-NH₂ | | | | | | | |
| 691 | 5-TAMRA-BaLTF$r8EYWAQL$AAAAAa- | 599 | | 2371.22 | | | | |
| | NH₂ | | | | | | | |
| 692 | 5-TAMRA-BaLTA$r8EYWAQL$AAAAAa- | 600 | | 2295.19 | | | | |
| | NH₂ | | | | | | | |
| 693 | 5-TAMRA- | 601 | | 2399.25 | | | | |
| | BaLTF$/r8EYWAQL$/AAAAAa-NH₂ | | | | | | | |
| 694 | Ac-LTF$r8EYWCou7QCba$SAA-OH | 602 | | 1947.93 | | | | |
| 695 | Ac-LTF$r8EYWCou7QCba$S-OH | 603 | | 1805.86 | | | | |
| 696 | Ac-LTA$r8EYWCou7QCba$SAA-NH₂ | 604 | | 1870.91 | | | | |
| 697 | Ac-LTF$r8EYACou7QCba$SAA-NH₂ | 605 | | 1831.9 | | | | |
| 698 | Ac-LTA$r8EYACou7QCba$SAA-NH₂ | 606 | | 1755.87 | | | | |
| 699 | Ac-LTF$/r8EYWCou7QCba$/SAA-NH₂ | 607 | | 1974.98 | | | | |
| 700 | Ac-LTF$r8EYWCou7QL$AAAAAa-NH₂ | 608 | | 2132.06 | | | | |
| 701 | Ac-LTF$/r8EYWCou7QL$/AAAAAa- | 609 | | 2160.09 | | | | |
| | NH₂ | | | | | | | |
| 702 | Ac-LTF$r8EYWCou7QL$AAAAA-OH | 610 | | 2062.01 | | | | |
| 703 | Ac-LTF$r8EYwCou7QL$AAAA-OH | 611 | | 1990.97 | | | | |
| 704 | Ac-LTF$r8EYwCou7QL$AAA-OH | 612 | | 1919.94 | | | | |
| 705 | Ac-LTF$r8EYWCou7QL$AA-OH | 613 | | 1848.9 | | | | |
| 706 | Ac-LTF$r8EYWCou7QL$A-OH | 614 | | 1777.86 | | | | |
| 707 | Ac-LTF$r8EYWAQL$AAAASa-NH₂ | 615 | iso2 | | 974.4 | | 973.53 | |
| 708 | Ac-LTF$r8AYWAAL$AAAAAa-NH₂ | 616 | iso2 | 1814.01 | 908.82 | 1815.02 | 908.01 | 605.68 |
| 709 | Biotin-BaLTF$r8EYWAQL$AAAAAa- | 617 | | 2184.14 | 1093.64 | 2185.15 | 1093.08 | 729.05 |
| | NH₂ | | | | | | | |
| 710 | Ac-LTF$r8HAWAQL$S-NH₂ | 618 | iso2 | 1505.84 | 754.43 | 1506.85 | 753.93 | 502.95 |
| 711 | Ac-LTF$r8EYWAQCba$SA-NH₂ | 619 | iso2 | 1672.89 | 838.05 | 1673.9 | 837.45 | 558.64 |
| 712 | Ac-LTF$r8HYWAQCba$SAA-NH₂ | 620 | iso2 | 1751.94 | 877.55 | 1752.95 | 876.98 | 584.99 |
| 713 | Ac-LTF$r8SYWAQCba$SAA-NH₂ | 621 | iso2 | 1701.91 | 852.48 | 1702.92 | 851.96 | 568.31 |
| 714 | Ac-LTF$r8RYWAQCba$SAA-NH₂ | 622 | iso2 | 1770.98 | 887.45 | 1771.99 | 886.5 | 591.33 |
| 715 | Ac-LTF$r8KYWAQCba$SAA-NH₂ | 623 | iso2 | 1742.98 | 872.92 | 1743.99 | 872.5 | 582 |
| 716 | Ac-LTF$r8EYWAQCba$AAA-NH₂ | 624 | iso2 | 1727.93 | 865.71 | 1728.94 | 864.97 | 576.98 |
| 717 | Ac-LTF$r8EYWAQL$AAAAAaBaC-NH₂ | 625 | | 2103.09 | 1053.12 | 2104.1 | 1052.55 | 702.04 |
| 718 | Ac-LTF$r8EYWAQL$AAAAAadPeg4C- | 626 | | 2279.19 | 1141.46 | 2280.2 | 1140.6 | 760.74 |
| | NH₂ | | | | | | | |
| 719 | Ac-LTA$r8AYWAAL$AAAAAa-NH₂ | 627 | | 1737.98 | 870.43 | 1738.99 | 870 | 580.33 |
| 720 | Ac-LTF$r8AYAAAL$AAAAAa-NH₂ | 628 | | 1698.97 | 851 | 1699.98 | 850.49 | 567.33 |
| 721 | 5-FAM-BaLTF$r8AYWAAL$AAAAAa- | 629 | | 2201.09 | 1101.87 | 2202.1 | 1101.55 | 734.7 |
| | NH₂ | | | | | | | |
| 722 | Ac-LTA$r8AYWAQL$AAAAAa-NH₂ | 630 | | 1795 | 898.92 | 1796.01 | 898.51 | 599.34 |
| 723 | Ac-LTF$r8AYAAQL$AAAAAa-NH₂ | 631 | | 1755.99 | 879.49 | 1757 | 879 | 586.34 |
| 724 | Ac-LTF$rda6AYWAAL$da5AAAAAa- | 632 | | 1807.97 | | 1808.98 | 904.99 | 603.66 |
| | NH₂ | | | | | | | |
| 725 | FITC-BaLTF$r8EYWAQL$AAAAAa-NH₂ | 633 | | 2347.1 | 1174.49 | 2348.11 | 1174.56 | 783.37 |
| 726 | FITC-BaLTF$r8EYWAQCba$SAA-NH₂ | 634 | | 2161.99 | 1082.35 | 2163 | 1082 | 721.67 |
| 733 | Ac-LTF$r8EYWAQL$EAAAAa-NH₂ | 635 | | 1987.05 | 995.03 | 1988.06 | 994.53 | 663.36 |
| 734 | Ac-LTF$r8AYWAQL$EAAAAa-NH₂ | 636 | | 1929.04 | 966.35 | 1930.05 | 965.53 | 644.02 |
| 735 | Ac-LTF$r8EYWAQL$AAAAAaBaKbio- | 637 | | 2354.25 | 1178.47 | 2355.26 | 1178.13 | 785.76 |
| | NH₂ | | | | | | | |
| 736 | Ac-LTF$r8AYWAAL$AAAAAa-NH₂ | 638 | | 1814.01 | 908.45 | 1815.02 | 908.01 | 605.68 |
| 737 | Ac-LTF$r8AYAAAL$AAAAAa-NH₂ | 639 | iso2 | 1698.97 | 850.91 | 1699.98 | 850.49 | 567.33 |
| 738 | Ac-LTF$r8AYAAQL$AAAAAa-NH₂ | 640 | iso2 | 1755.99 | 879.4 | 1757 | 879 | 586.34 |
| 739 | Ac-LTF$r8EYWAQL$EAAAAa-NH₂ | 641 | iso2 | 1987.05 | 995.21 | 1988.06 | 994.53 | 663.36 |
| 740 | Ac-LTF$r8AYWAQL$EAAAAa-NH₂ | 642 | iso2 | 1929.04 | 966.08 | 1930.05 | 965.53 | 644.02 |
| 741 | Ac-LTF$r8EYWAQCba$SAAAAa-NH₂ | 643 | | 1957.04 | 980.04 | 1958.05 | 979.53 | 653.35 |
| 742 | Ac-LTF$r8EYWAQLStAAA$r5AA-NH₂ | 644 | | 2023.12 | 1012.83 | 2024.13 | 1012.57 | 675.38 |
| 743 | Ac-LTF$r8EYWAQL$A$AAA$A-NH₂ | 645 | | 2108.17 | 1055.44 | 2109.18 | 1055.09 | 703.73 |
| 744 | Ac-LTF$r8EYWAQL$AA$AAA$A-NH₂ | 646 | | 2179.21 | 1090.77 | 2180.22 | 1090.61 | 727.41 |
| 745 | Ac-LTF$r8EYWAQL$AAA$AAA$A-NH₂ | 647 | | 2250.25 | 1126.69 | 2251.26 | 1126.13 | 751.09 |
| 746 | Ac-AAALTF$r8EYWAQL$AAA-OH | 648 | | 1930.02 | | 1931.03 | 966.02 | 644.35 |
| 747 | Ac-AAALTF$r8EYWAQL$AAA-NH₂ | 649 | | 1929.04 | 965.85 | 1930.05 | 965.53 | 644.02 |
| 748 | Ac-AAAALTF$r8EYWAQL$AAA-NH₂ | 650 | | 2000.08 | 1001.4 | 2001.09 | 1001.05 | 667.7 |
| 749 | Ac-AAAAALTF$r8EYwAQL$AAA-NH₂ | 651 | | 2071.11 | 1037.13 | 2072.12 | 1036.56 | 691.38 |
| 750 | Ac-AAAAAALTF$r8EYwAQL$AAA-NH₂ | 652 | | 2142.15 | | 2143.16 | 1072.08 | 715.06 |
| 751 | Ac-LTF$rda6EYWAQCba$da6SAA-NH₂ | 653 | iso2 | 1751.89 | 877.36 | 1752.9 | 876.95 | 584.97 |
| 752 | Ac-t$r5wya$r5f4CF3ekllr-NH₂ | 654 | | | 844.25 | | | |
| 753 | Ac-tawy$r5nf4CF3e$r5llr-NH₂ | 655 | | | 837.03 | | | |
| 754 | Ac-tawya$r5f4CF3ek$r5lr-NH₂ | 656 | | | 822.97 | | | |
| 755 | Ac-tawyanf4CF3e$r5llr$r5a-NH₂ | 657 | | | 908.35 | | | |
| 756 | Ac-t$s8wyanf4CF3e$r5llr-NH₂ | 658 | | | 858.03 | | | |
| 757 | Ac-tawy$s8nf4CF3ekll$r5a-NH₂ | 659 | | | 879.86 | | | |
| 758 | Ac-tawya$s8f4CF3ekllr$r5a-NH₂ | 660 | | | 936.38 | | | |
| 759 | Ac-tawy$s8naekll$r5a-NH₂ | 661 | | | 844.25 | | | |
| 760 | 5-FAM-Batawy$s8nf4CF3ekll$r5a- | 662 | | | | | | |
| | NH₂ | | | | | | | |
| 761 | 5-FAM-Batawy$s8naekll$r5a-NH₂ | 663 | | | | | | |
| 762 | Ac-tawy$s8nf4CF3eall$r5a-NH₂ | 664 | | | | | | |
| 763 | Ac-tawy$s8nf4CF3ekll$r5aaaaa- | 665 | | | | | | |
| | NH₂ | | | | | | | |
| 764 | Ac-tawy$s8nf4CF3eall$r5aaaaa- | 666 | | | | | | |
| | NH₂ | | | | | | | |

TABLE 1a shows a selection of peptidomimetic macrocycles.

TABLE 1a
TABLE 1a shows a selection of peptidomimetic macrocycles.

| | | SEQ | | | | Calc | Calc | Calc |
|---|---|---|---|---|---|---|---|---|
| | | ID | | Exact | Found | (M + | (M + | (M + |
| SP | Sequence | NO: | Isomer | Mass | Mass | 1)/1 | 2)/2 | 3)/3 |
| 244 | Ac-LTF$r8EF4coohWAQCba$SANleA- | 667 | | 1885 | 943.59 | 1886.01 | 943.51 | 629.34 |
| | NH₂ | | | | | | | |
| 331 | Ac-LTF$r8EYWAQL$AAAAAa-NH₂ | 668 | iso2 | 1929.04 | 966.08 | 1930.05 | 965.53 | 644.02 |
| 555 | Ac-LTF$r8EY6clWAQL$AAAAAa-NH₂ | 669 | | 1963 | 983.28 | 1964.01 | 982.51 | 655.34 |
| 557 | Ac-AAALTF$r8EYWAQL$AAAAAa-NH₂ | 670 | | 2142.15 | 1072.83 | 2143.16 | 1072.08 | 715.06 |
| 558 | Ac-LTF34F2$r8EYWAQL$AAAAAa-NH₂ | 671 | | 1965.02 | 984.3 | 1966.03 | 983.52 | 656.01 |
| 562 | Ac-LTF$r8EYWAQL$AAibAAAa-NH₂ | 672 | | 1943.06 | 973.11 | 1944.07 | 972.54 | 648.69 |
| 564 | Ac-LTF$r8EYWAQL$AAAAibAa-NH₂ | 673 | | 1943.06 | 973.48 | 1944.07 | 972.54 | 648.69 |
| 566 | Ac-LTF$r8EYWAQL$AAAAAiba-NH₂ | 674 | iso2 | 1943.06 | 973.38 | 1944.07 | 972.54 | 648.69 |
| 567 | Ac-LTF$r8EYWAQL$AAAAAAib-NH₂ | 675 | | 1943.06 | 973.01 | 1944.07 | 972.54 | 648.69 |
| 572 | Ac-LTF$r8EYWAQL$AAAAaa-NH₂ | 676 | | 1929.04 | 966.35 | 1930.05 | 965.53 | 644.02 |
| 573 | Ac-LTF$r8EYWAQL$AAAAAA-NH₂ | 677 | | 1929.04 | 966.35 | 1930.05 | 965.53 | 644.02 |
| 578 | Ac-LTF$r8EYWAQL$AAAAASar-NH₂ | 678 | | 1929.04 | 966.08 | 1930.05 | 965.53 | 644.02 |
| 551 | Ac-LTF$r8EYWAQL$AAAAAa-OH | 679 | iso2 | 1930.02 | 965.89 | 1931.03 | 966.02 | 644.35 |
| 662 | Ac-LTF$rda6AYWAQL$da5AAAAAa- | 680 | | 1974.06 | 934.44 | | 933.49 | |
| | NH₂ | | | | | | | |
| 367 | 5-FAM-BaLTF$r8EYWAQCba$SAA-NH₂ | 681 | | 2131 | 1067.09 | 2132.01 | 1066.51 | 711.34 |
| 349 | Ac-LTF$r8EF4coohWAQCba$AAAAAa- | 682 | iso2 | 1969.04 | 986.06 | 1970.05 | 985.53 | 657.35 |
| | NH₂ | | | | | | | |
| 347 | Ac-LTF$r8EYWAQCba$AAAAAa-NH₂ | 683 | iso2 | 1941.04 | 972.55 | 1942.05 | 971.53 | 648.02 |

TABLE 1b shows a further selection of peptidomimetic macrocycles.

TABLE 1b
TABLE 1b shows a further selection of peptidomimetic macrocycles.

| | | SEQ | | | | Calc | Calc | Calc |
|---|---|---|---|---|---|---|---|---|
| | | ID | | Exact | Found | (M + | (M + | (M + |
| SP | Sequence | NO: | Isomer | Mass | Mass | 1)/1 | 2)/2 | 3)/3 |
| 581 | Ac-TF$r8EYWAQL$AAAAAa-NH₂ | 684 | | 1815.96 | 929.85 | 1816.97 | 908.99 | 606.33 |
| 582 | Ac-F$r8EYWAQL$AAAAAa-NH₂ | 685 | | 1714.91 | 930.92 | 1715.92 | 858.46 | 572.64 |
| 583 | Ac-LVF$r8EYWAQL$AAAAAa- | 686 | | 1927.06 | 895.12 | 1928.07 | 964.54 | 643.36 |
| | NH₂ | | | | | | | |
| 584 | Ac-AAF$r8EYWAQL$AAAAAa- | 687 | | 1856.98 | 859.51 | 1857.99 | 929.5 | 620 |
| | NH₂ | | | | | | | |
| 585 | Ac-LTF$r8EYWAQL$AAAAa-NH₂ | 688 | | 1858 | 824.08 | 1859.01 | 930.01 | 620.34 |
| 586 | Ac-LTF$r8EYWAQL$AAAa-NH₂ | 689 | | 1786.97 | 788.56 | 1787.98 | 894.49 | 596.66 |
| 587 | Ac-LTF$r8EYWAQL$AAa-NH₂ | 690 | | 1715.93 | 1138.57 | 1716.94 | 858.97 | 572.98 |
| 588 | Ac-LTF$r8EYwAQL$Aa-NH₂ | 691 | | 1644.89 | 1144.98 | 1645.9 | 823.45 | 549.3 |
| 589 | Ac-LTF$r8EYWAQL$a-NH₂ | 692 | | 1573.85 | 1113.71 | 1574.86 | 787.93 | 525.62 |

In the sequences shown above and elsewhere, the following abbreviations are used: “Nle” represents norleucine, “Aib” represents 2-aminoisobutyric acid, “Ac” represents acetyl, and “Pr” represents propionyl. Amino acids represented as “$” are alpha-Me S5-pentenyl-alanine olefin amino acids connected by an all-carbon crosslinker comprising one double bond. Amino acids represented as “$r5” are alpha-Me R5-pentenyl-alanine olefin amino acids connected by an all-carbon comprising one double bond. Amino acids represented as “$s8” are alpha-Me S8-octenyl-alanine olefin amino acids connected by an all-carbon crosslinker comprising one double bond. Amino acids represented as “$r8” are alpha-Me R8-octenyl-alanine olefin amino acids connected by an all-carbon crosslinker comprising one double bond. “Ahx” represents an aminocyclohexyl linker.

The crosslinkers are linear all-carbon crosslinker comprising eight or eleven carbon atoms between the alpha carbons of each amino acid. Amino acids represented as “$/” are alpha-Me S5-pentenyl-alanine olefin amino acids that are not connected by any crosslinker. Amino acids represented as “$/r5” are alpha-Me R5-pentenyl-alanine olefin amino acids that are not connected by any crosslinker. Amino acids represented as “$/s8” are alpha-Me S8-octenyl-alanine olefin amino acids that are not connected by any crosslinker. Amino acids represented as “$/r8” are alpha-Me R8-octenyl-alanine olefin amino acids that are not connected by any crosslinker.

Amino acids represented as “Amw” are alpha-Me tryptophan amino acids. Amino acids represented as “Aml” are alpha-Me leucine amino acids. Amino acids represented as “Amf” are alpha-Me phenylalanine amino acids. Amino acids represented as “2ff” are 2-fluoro-phenylalanine amino acids. Amino acids represented as “3ff” are 3-fluoro-phenylalanine amino acids. Amino acids represented as “St” are amino acids comprising two pentenyl-alanine olefin side chains, each of which is crosslinked to another amino acid as indicated. Amino acids represented as “St//” are amino acids comprising two pentenyl-alanine olefin side chains that are not crosslinked. Amino acids represented as “% St” are amino acids comprising two pentenyl-alanine olefin side chains, each of which is crosslinked to another amino acid as indicated via fully saturated hydrocarbon crosslinks. Amino acids represented as “Ba” are beta-alanine. The lower-case character “e” or “z” within the designation of a crosslinked amino acid (e.g. “$er8” or “$zr8”) represents the configuration of the double bond (E or Z, respectively). In other contexts, lower-case letters such as “a” or “f” represent D amino acids (e.g. D-alanine, or D-phenylalanine, respectively).

Amino acids designated as “NmW” represent N-methyltryptophan. Amino acids designated as “NmY” represent N-methyltyrosine. Amino acids designated as “NmA” represent N-methylalanine. “Kbio” represents a biotin group attached to the side chain amino group of a lysine residue. Amino acids designated as “Sar” represent sarcosine. Amino acids designated as “Cha” represent cyclohexyl alanine. Amino acids designated as “Cpg” represent cyclopentyl glycine. Amino acids designated as “Chg” represent cyclohexyl glycine. Amino acids designated as “Cba” represent cyclobutyl alanine. Amino acids designated as “F4I” represent 4-iodo phenylalanine. “7L” represents N15 isotopic leucine. Amino acids designated as “F3Cl” represent 3-chloro phenylalanine. Amino acids designated as “F4cooh” represent 4-carboxy phenylalanine. Amino acids designated as “F34F2” represent 3,4-difluoro phenylalanine. Amino acids designated as “6clW” represent 6-chloro tryptophan. Amino acids designated as “$rda6” represent alpha-Me R6-hexynyl-alanine alkynyl amino acids, crosslinked via a dialkyne bond to a second alkynyl amino acid.

Amino acids designated as “$da5” represent alpha-Me S5-pentynyl-alanine alkynyl amino acids, wherein the alkyne forms one half of a dialkyne bond with a second alkynyl amino acid. Amino acids designated as “$ra9” represent alpha-Me R9-nonynyl-alanine alkynyl amino acids, crosslinked via an alkyne metathesis reaction with a second alkynyl amino acid. Amino acids designated as “$a6” represent alpha-Me S6-hexynyl-alanine alkynyl amino acids, crosslinked via an alkyne metathesis reaction with a second alkynyl amino acid. The designation “iso1” or “iso2” indicates that the peptidomimetic macrocycle is a single isomer.

Amino acids designated as “Cit” represent citrulline. Amino acids designated as “Cou4”, “Cou6”, “Cou7” and “Cou8”, respectively, represent the following structures:

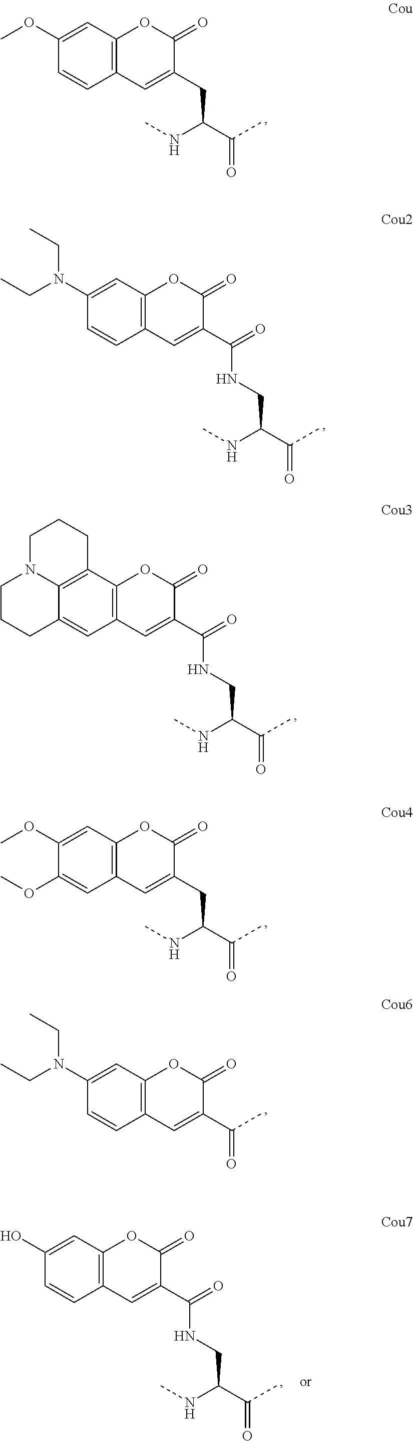

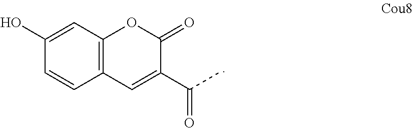

In some embodiments, a peptidomimetic macrocycle is obtained in more than one isomer, for example due to the configuration of a double bond within the structure of the crosslinker (E vs Z). Such isomers can or cannot be separable by conventional chromatographic methods. In some embodiments, one isomer has improved biological properties relative to the other isomer. In one embodiment, an E crosslinker olefin isomer of a peptidomimetic macrocycle has better solubility, better target affinity, better in vivo or in vitro efficacy, higher helicity, or improved cell permeability relative to its Z counterpart. In another embodiment, a Z crosslinker olefin isomer of a peptidomimetic macrocycle has better solubility, better target affinity, better in vivo or in vitro efficacy, higher helicity, or improved cell permeability relative to its E counterpart.

TABLE 1c shows exemplary peptidomimetic macrocycles.

TABLE 1c

| | SEQ | |
|---|---|---|
| | ID | |
| SP# | NO: | Structure |
| 154 | 163 | 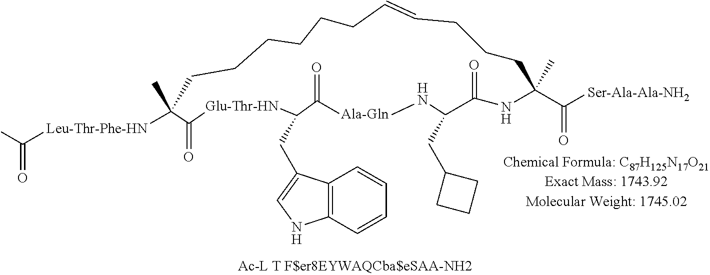 |
| 115 | 124 | 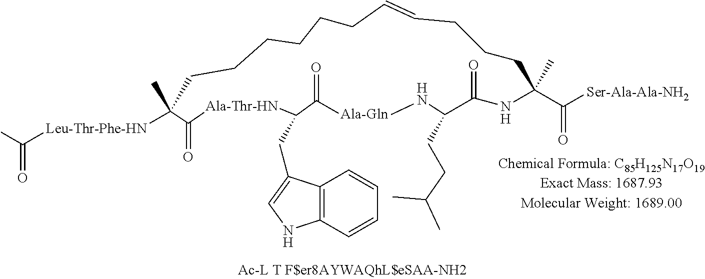 |
| 114 | 123 | 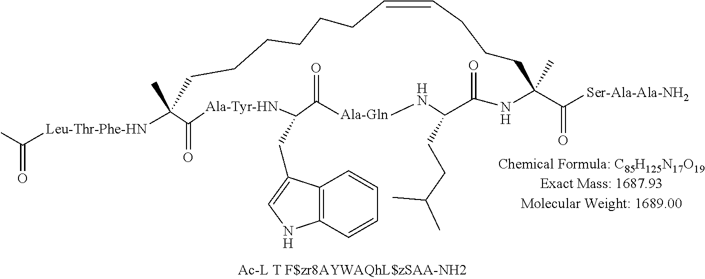 |
| 99 | 108 | 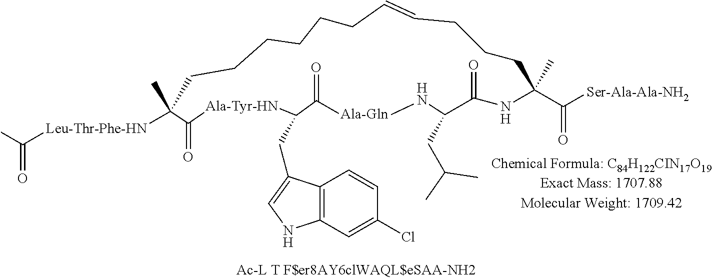 |
| 388 | 397 | 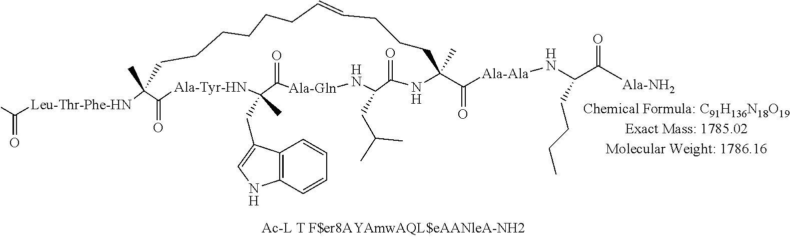 |
| 331 | 340 | 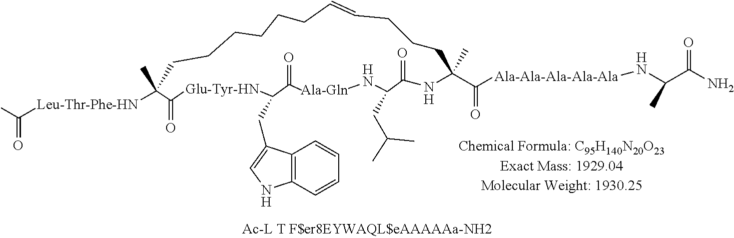 |
| 445 | 454 | 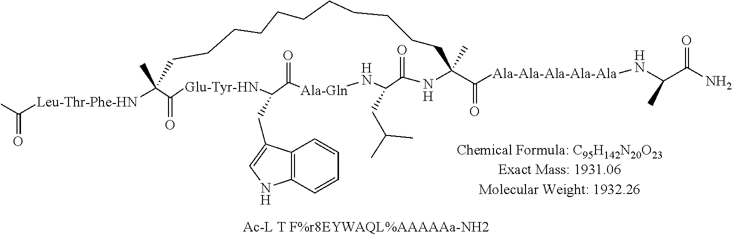 |
| 351 | 360 | 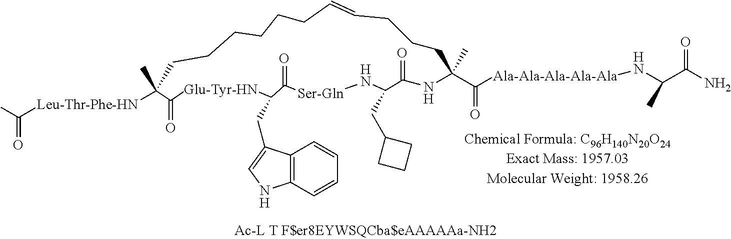 |
| 71 | 80 | 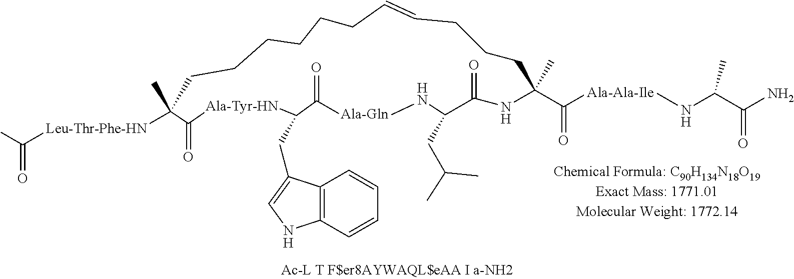 |
| 69 | 78 | 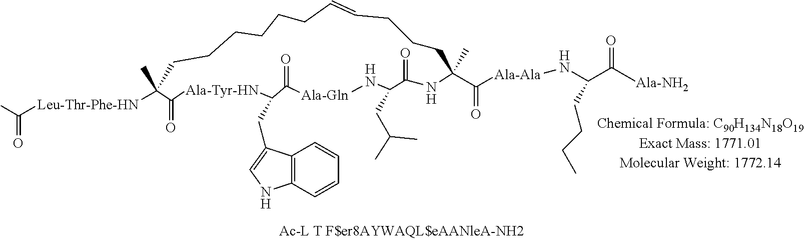 |
| 7 | 16 | 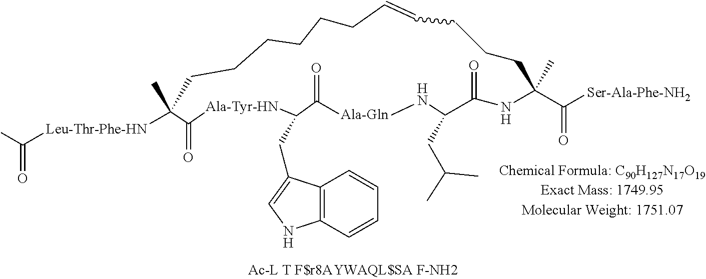 |
| 160 | 169 | 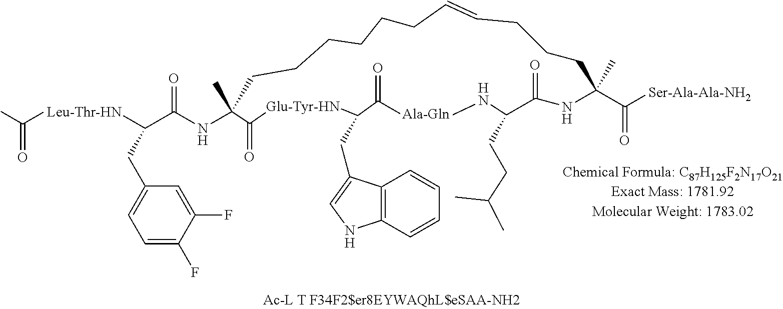 |
| 315 | 324 | 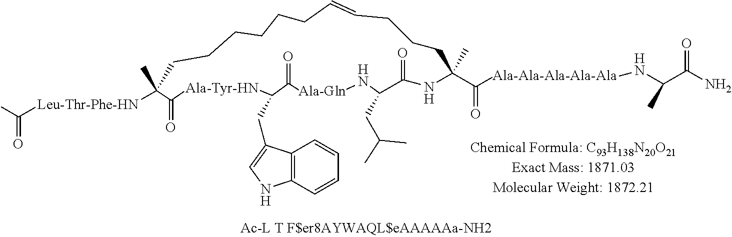 |
| 249 | 258 | 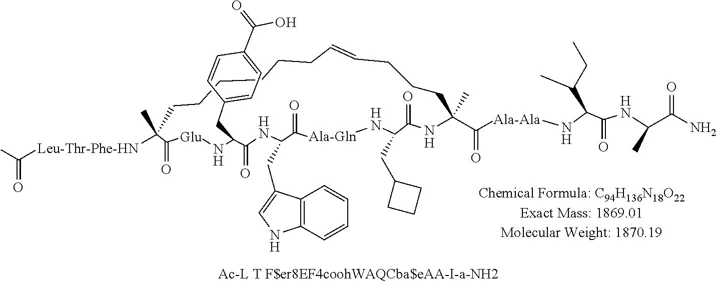 |
| 437 | 446 | 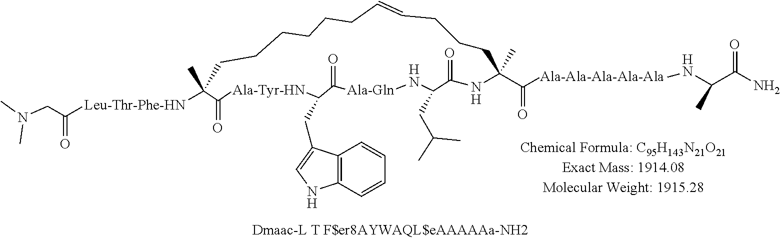 |
| 349 | 358 | 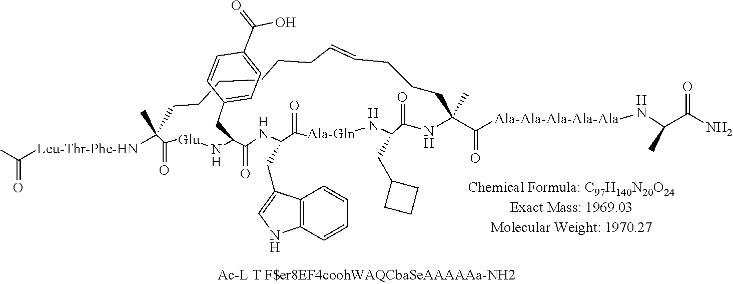 |
| 555 | 464 | 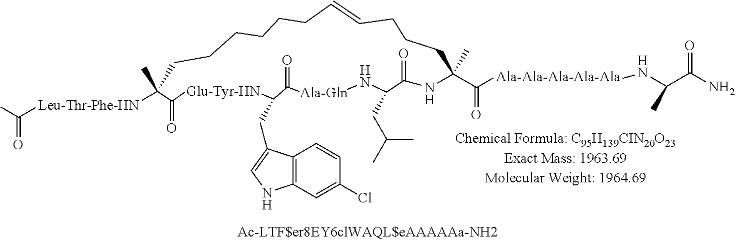 |
| 557 | 466 | 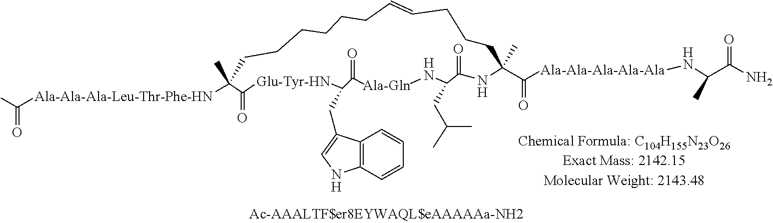 |
| 558 | 467 | 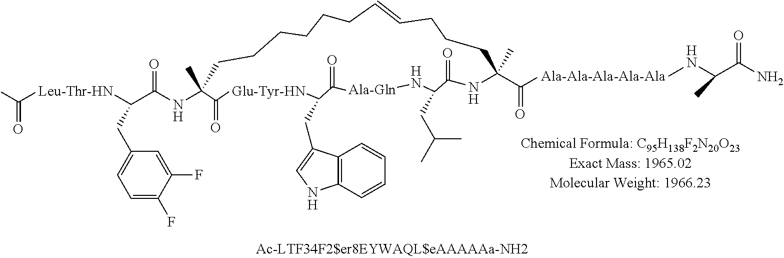 |
| 367 | 376 | 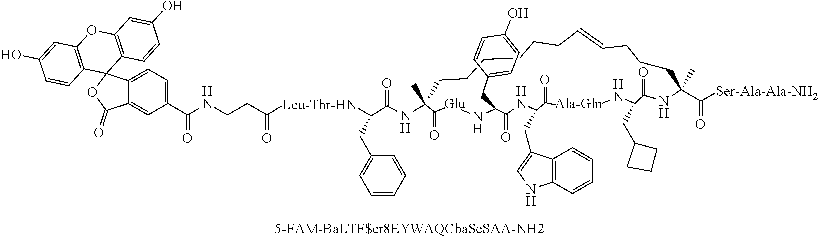 |
| 562 | 471 | 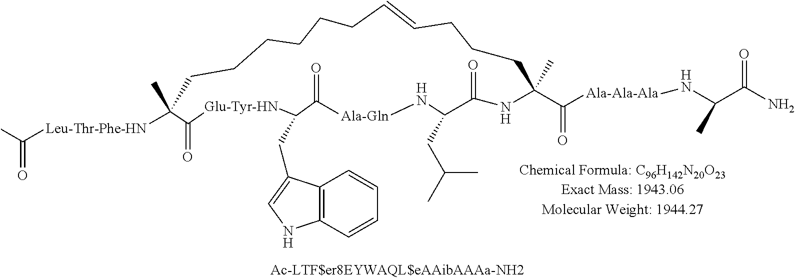 |
| 564 | 473 | 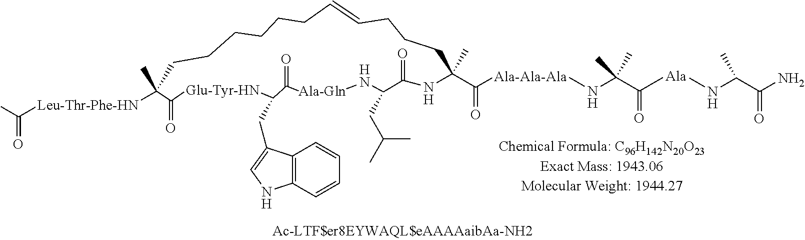 |
| 566 | 475 | 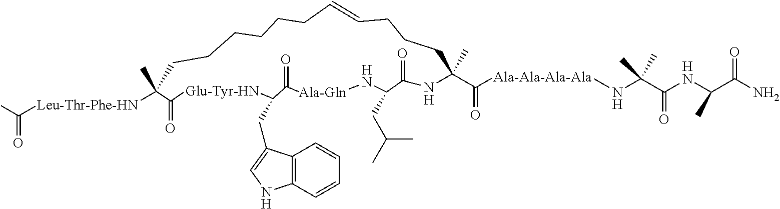 |
| 567 | 476 | 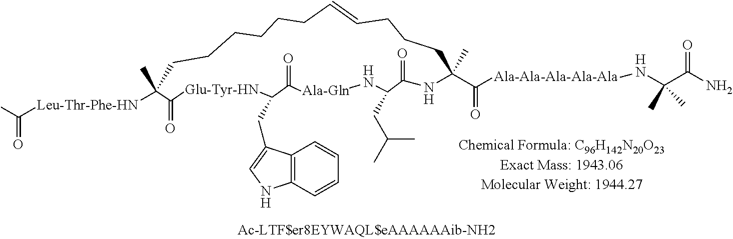 |
| 572 | 481 | 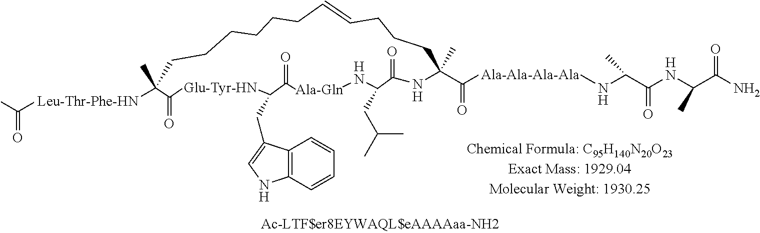 |
| 573 | 482 | 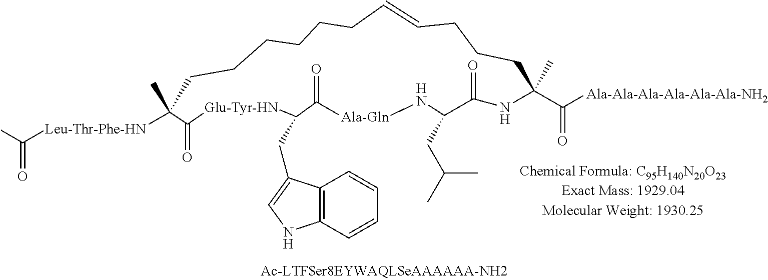 |
| 578 | 487 | 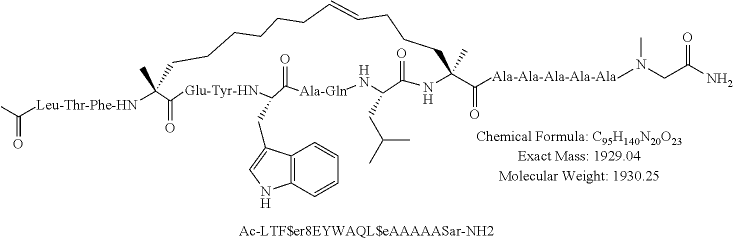 |
| 664 | 572 | 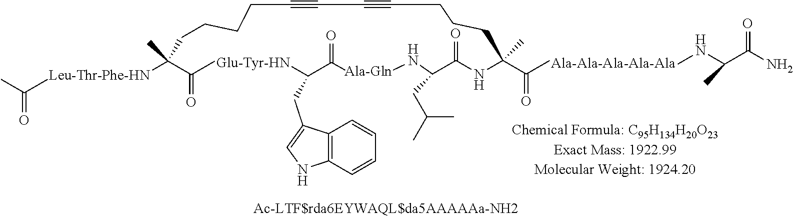 |
| 662 | 572 | 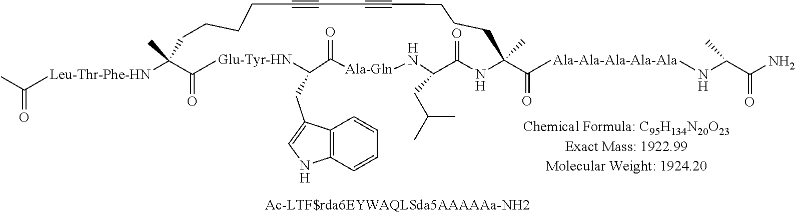 |
| | 1500 | 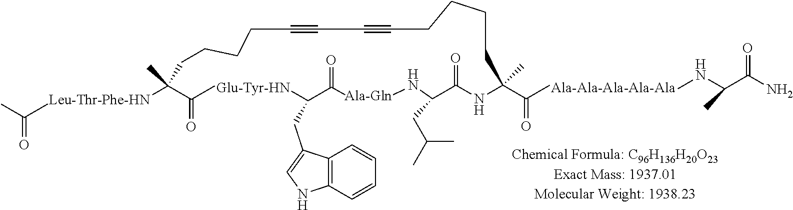 |

In some embodiments, peptidomimetic macrocycles exclude peptidomimetic macrocycles shown in TABLE 2a:

TABLE 2a

| Sequence | SEQ ID NO: |
|---|---|
| L$r5QETESD$s8WKLLPEN | 693 |
| LSQ$r5TESDLW$s8LLPEN | 694 |
| LSQE$r5FSDLWK$s8LPEN | 695 |
| LSQET$r5SDLWKL$s8PEN | 696 |
| LSQETF$r5DLWKLL$s8EN | 697 |
| LXQETES$r5LWKLLP$s8N | 698 |
| LSQETESD$r5WKLLPE$s8 | 699 |
| LSQQTF$r5DLWKLL$s8EN | 700 |
| LSQETF$r5DLWKLL$s8QN | 701 |
| LSQQTF$r5DLWKLL$s8QN | 702 |
| LSQETF$r5NLWKLL$s8QN | 703 |
| LSQQTF$r5NLWKLL$s8QN | 704 |
| LSQQTF$r5NLWRLL$s8QN | 705 |
| QSQQTF$r5NLWKLL$s8QN | 706 |
| QSQQTF$r5NLWRLL$s8QN | 707 |
| QSQQTA$r5NLWRLL$s8QN | 708 |
| L$r8QETFSD$WKLLPEN | 709 |
| LSQ$r8TFSDLW$LLPEN | 710 |
| LSQE$r8FSDLWK$LPEN | 711 |
| LSQET$r8SDLWKL$PEN | 712 |
| LSQETF$r8DLWKLL$EN | 713 |
| LXQETFS$r8LWKLLP$N | 714 |
| LSQETFSD$r8WKLLPE$ | 715 |
| LSQQTF$r8DLWKLL$EN | 716 |
| LSQETF$r8DLWKLL$QN | 717 |
| LSQQTF$r8DLWKLL$QN | 718 |
| LSQETF$r8NLWKLL$QN | 719 |
| LSQQTF$r8NLWKLL$QN | 720 |
| LSQQTF$r8NLWRLL$QN | 721 |
| QSQQTF$r8NLWKLL$QN | 722 |
| QSQQTF$r8NLWRLL$QN | 723 |
| QSQQTA$r8NLWRLL$QN | 724 |
| QSQQTF$r8NLWRKK$QN | 725 |
| QQTF$r8DLWRLL$EN | 726 |
| QQTF$r8DLWRLL$ | 727 |
| LSQQTF$DLW$LL | 728 |
| QQTF$DLW$LL | 729 |
| QQTA$r8DLWRLL$EN | 730 |
| QSQQTF$r5NLWRLL$s8QN | 731 |
| (dihydroxylated olefin) | |
| QSQQTA$r5NLWRLL$s8QN | 732 |
| (dihydroxylated olefin) | |
| QSQQTF$r8DLWRLL$QN | 733 |
| QTF$r8NLWRLL$ | 734 |
| QSQQTF$NLW$LLPQN | 735 |
| QS$QTF$NLWRLLPQN | 736 |
| $TFS$LWKLL | 737 |
| ETF$DLW$LL | 738 |
| QTF$NLW$LL | 739 |
| $SQE$FSNLWKLL | 740 |

In TABLE 2a, the peptides can comprise an N-terminal capping group such as acetyl or an additional linker such as beta-alanine between the capping group and the start of the peptide sequence.

In some embodiments, peptidomimetic macrocycles do not comprise a peptidomimetic macrocycle structure as shown in TABLE 2a.

In some embodiments, peptidomimetic macrocycles exclude those shown in TABLE 2b:

TABLE 2b

| | | SEQ | | | Observed mass |
|---|---|---|---|---|---|
| SP | Sequence | ID NO: | Exact Mass | M + 2 | (m/e) |
| 1 | Ac-LSQETF$r8DLWKLL$EN-NH₂ | 741 | 2068.13 | 1035.07 | 1035.36 |
| 2 | Ac-LSQETF$r8NLWKLL$QN-NH₂ | 742 | 2066.16 | 1034.08 | 1034.31 |
| 3 | Ac-LSQQTF$r8NLWRLL$QN-NH₂ | 743 | 2093.18 | 1047.59 | 1047.73 |
| 4 | Ac-QSQQTF$r8NLWKLL$QN-NH₂ | 744 | 2080.15 | 1041.08 | 1041.31 |
| 5 | Ac-QSQQTF$r8NLWRLL$QN-NH₂ | 745 | 2108.15 | 1055.08 | 1055.32 |
| 6 | Ac-QSQQTA$r8NLWRLL$QN-NH₂ | 746 | 2032.12 | 1017.06 | 1017.24 |
| 7 | Ac-QAibQQTF$r8NLWRLL$QN-NH₂ | 747 | 2106.17 | 1054.09 | 1054.34 |
| 8 | Ac-QSQQTFSNLWRLLPQN-NH₂ | 748 | 2000.02 | 1001.01 | 1001.26 |
| 9 | Ac-QSQQTF$/r8NLwRLLVQN-NH₂ | 749 | 2136.18 | 1069.09 | 1069.37 |
| 10 | Ac-QSQAibTF$r8NLWRLL$QN-NH₂ | 750 | 2065.15 | 1033.58 | 1033.71 |
| 11 | Ac-QSQQTF$r8NLWRLL$AN-NH₂ | 751 | 2051.13 | 1026.57 | 1026.70 |
| 12 | Ac-ASQQTF$r8NLWRLL$QN-NH₂ | 752 | 2051.13 | 1026.57 | 1026.90 |
| 13 | Ac-QSQQTF$r8ALWRLL$QN-NH₂ | 753 | 2065.15 | 1033.58 | 1033.41 |
| 14 | Ac-QSQETF$r8NLWRLL$QN-NH₂ | 754 | 2109.14 | 1055.57 | 1055.70 |
| 15 | Ac-RSQQTF$r8NLWRLL$QN-NH₂ | 755 | 2136.20 | 1069.10 | 1069.17 |
| 16 | Ac-RSQQTF$r8NLWRLL$EN-NH₂ | 756 | 2137.18 | 1069.59 | 1069.75 |
| 17 | Ac-LSQETFSDLWKLLPEN-NH₂ | 757 | 1959.99 | 981.00 | 981.24 |
| 18 | Ac-QSQ$TFS$LWRLLPQN-NH₂ | 758 | 2008.09 | 1005.05 | 1004.97 |
| 19 | Ac-QSQQ$FSN$WRLLPQN-NH₂ | 759 | 2036.06 | 1019.03 | 1018.86 |
| 20 | Ac-QSQQT$SNL$RLLPQN-NH₂ | 760 | 1917.04 | 959.52 | 959.32 |
| 21 | Ac-QSQQTF$NLW$LLPQN-NH₂ | 761 | 2007.06 | 1004.53 | 1004.97 |
| 22 | Ac-RTQATF$r8NQWAibANle$TNAibTR-NH₂ | 762 | 2310.26 | 1156.13 | 1156.52 |
| 23 | Ac-QSQQTF$r8NLWRLL$RN-NH₂ | 763 | 2136.20 | 1069.10 | 1068.94 |
| 24 | Ac-QSQRTF$r8NLWRLL$QN-NH₂ | 764 | 2136.20 | 1069.10 | 1068.94 |
| 25 | Ac-QSQQTF$r8NNleWRLL$QN-NH₂ | 765 | 2108.15 | 1055.08 | 1055.44 |
| 26 | Ac-QSQQTF$r8NLWRNleL$QN-NH₂ | 766 | 2108.15 | 1055.08 | 1055.84 |
| 27 | Ac-QSQQTF$r8NLWRLNle$QN-NH₂ | 767 | 2108.15 | 1055.08 | 1055.12 |
| 28 | Ac-QSQQTY$r8NLWRLL$QN-NH₂ | 768 | 2124.15 | 106108 | 1062.92 |
| 29 | Ac-RAibQQTF$r8NLWRLL$QN-NH₂ | 769 | 2134.22 | 1068.11 | 1068.65 |
| 30 | Ac-MPRFMDYWEGLN-NH₂ | 770 | 1598.70 | 800.35 | 800.45 |
| 31 | Ac-RSQQRF$r8NLwRLL$QN-NH₂ | 771 | 2191.25 | 1096.63 | 1096.83 |
| 32 | Ac-QSQQRF$r8NLWRLL$QN-NH₂ | 772 | 2163.21 | 1082.61 | 1082.87 |
| 33 | Ac-RAibQQRF$r8NLWRLL$QN-NH₂ | 773 | 2189.27 | 1095.64 | 1096.37 |
| 34 | Ac-RSQQRF$r8NFwRLL$QN-NH₂ | 774 | 2225.23 | 1113.62 | 1114.37 |
| 35 | Ac-RSQQRF$r8NYWRLL$QN-NH₂ | 775 | 2241.23 | 1121.62 | 1122.37 |
| 36 | Ac-RSQQTF$r8NLWQLL$QN-NH₂ | 776 | 2108.15 | 1055.08 | 1055.29 |
| 37 | Ac-QSQQTF$r8NLWQAmlL$QN-NH₂ | 777 | 2094.13 | 1048.07 | 1048.32 |
| 38 | Ac-QSQQTF$r8NAmlWRLL$QN-NH₂ | 778 | 2122.17 | 1062.09 | 1062.35 |
| 39 | Ac-NlePRF$r8DYWEGL$QN-NH₂ | 779 | 1869.98 | 935.99 | 936.20 |
| 40 | Ac-NlePRF$r8NYWRLL$QN-NH₂ | 780 | 1952.12 | 977.06 | 977.35 |
| 41 | Ac-RF$r8NLWRLL$Q-NH₂ | 781 | 1577.96 | 789.98 | 790.18 |
| 42 | Ac-QSQQTF$r8N2ffWRLL$QN-NH₂ | 782 | 2160.13 | 1081.07 | 1081.40 |
| 43 | Ac-QSQQTF$r8N3ffWRLL$QN-NH₂ | 783 | 2160.13 | 1081.07 | 1081.34 |
| 44 | Ac-QSQQTF#r8NLWRLL#QN-NH₂ | 784 | 2080.12 | 1041.06 | 1041.34 |
| 45 | Ac-RSQQTA$r8NLWRLL$QN-NH₂ | 785 | 2060.16 | 1031.08 | 1031.38 |
| 46 | Ac-QSQQTF%r8NLWRLL%QN-NH₂ | 786 | 2110.17 | 1056.09 | 1056.55 |
| 47 | HepQSQ$TFSNLWRLLPQN-NH₂ | 787 | 2051.10 | 1026.55 | 1026.82 |
| 48 | HepQSQ$TF$r8NLWRLL$QN-NH₂ | 788 | 2159.23 | 1080.62 | 1080.89 |
| 49 | Ac-QSQQTF$r8NL6clWRLL$QN-NH₂ | 789 | 2142.11 | 1072.06 | 1072.35 |
| 50 | Ac-QSQQTF$r8NLMe6c1wRLL$QN-NH₂ | 790 | 2156.13 | 1079.07 | 1079.27 |
| 51 | Ac-LTFEHYWAQLTS-NH₂ | 791 | 1535.74 | 768.87 | 768.91 |
| 52 | Ac-LTF$HYW$QLTS-NH₂ | 792 | 1585.83 | 791.92 | 761.67 |
| 53 | Ac-LTFE$YWA$LTS-NH₂ | 793 | 1520.79 | 761.40 | 761.67 |
| 54 | Ac-LTF$zr8HYWAQL$zS-NH₂ | 794 | 1597.87 | 799.94 | 800.06 |
| 55 | Ac-LTF$r8HYWRQL$S-NH₂ | 795 | 1682.93 | 842.47 | 842.72 |
| 56 | Ac-QS$QTFStNLWRLL$s8QN-NH₂ | 796 | 2145.21 | 1071.61 | 1073.90 |
| 57 | Ac-QSQQTASNLWRLLPQN-NH₂ | 797 | 1923.99 | 961.00 | 961.26 |
| 58 | Ac-QSQQTA$/r8NLwRLL$NN-NH₂ | 798 | 2060.15 | 1031.08 | 1031.24 |
| 59 | Ac-ASQQTF$/r8NLwRLL$NN-NH₂ | 799 | 2079.16 | 1040.58 | 1040.89 |
| 60 | Ac-$SQQ$FSNLWRLLAibQN-NH₂ | 800 | 2009.09 | 1005.55 | 1005.86 |
| 61 | Ac-QS$QTF$NLwRLLAibQN-NH₂ | 801 | 2023.10 | 1012.55 | 1012.79 |
| 62 | Ac-QSQQ$FSN$WRLLAibQN-NH₂ | 802 | 2024.06 | 1013.03 | 1011.31 |
| 63 | Ac-QSQQTF$NLW$LLAibQN-NH₂ | 803 | 1995.06 | 998.53 | 998.87 |
| 64 | Ac-QSQQTFS$LWR$LAibQN-NH₂ | 804 | 2011.06 | 1006.53 | 1006.83 |
| 65 | Ac-QSQQTFSNLW$LLA$N-NH₂ | 805 | 1940.02 | 971.01 | 971.29 |
| 66 | Ac-$/SQQ$/FSNLWRLLAibQN-NH₂ | 806 | 2037.12 | 1019.56 | 1019.78 |
| 67 | Ac-QS$NTFVNLwRLLAibQN-NH₂ | 807 | 2051.13 | 1026.57 | 1026.90 |
| 68 | Ac-QSQQ$/FSN$/WRLLAibQN-NH₂ | 808 | 2052.09 | 1027.05 | 1027.36 |
| 69 | Ac-QSQQTFVNLWVLLAibQN-NH₂ | 809 | 2023.09 | 1012.55 | 1013.82 |
| 70 | Ac-QSQ$TFS$LWRLLAibQN-NH₂ | 810 | 1996.09 | 999.05 | 999.39 |
| 71 | Ac-QSQ$/TFS$/LWRLLAibQN-NH₂ | 811 | 2024.12 | 1011.06 | 1011.37 |
| 72 | Ac-QS$/QTFSt//NLWRLL$/s8QN-NH₂ | 812 | 2201.27 | 1101.64 | 1101.00 |
| 73 | Ac-$r8SQQTFS$LWRLLAibQN-NH₂ | 813 | 2038.14 | 1020.07 | 1020.23 |
| 74 | Ac-QSQ$r8TFSNLW$LLAibQN-NH₂ | 814 | 1996.08 | 999.04 | 999.32 |
| 75 | Ac-QSQQTFS$r8LWRLLA$N-NH₂ | 815 | 2024.12 | 1011.06 | 1011.37 |
| 76 | Ac-QS$r5QTFStNLW$LLAibQN-NH₂ | 816 | 2032.12 | 1017.06 | 1017.39 |
| 77 | Ac-$/r8SQQTFS$/LWRLLAibQN-NH₂ | 817 | 2066.17 | 1034.09 | 1034.80 |
| 78 | Ac-QSQ$/r8TFSNLW$/LLAibQN-NH₂ | 818 | 2024.11 | 1011.06 | 1014.34 |
| 79 | Ac-QSQQTFS$/r8LWRLLA$/N-NH₂ | 819 | 2052.15 | 1027.08 | 1027.16 |
| 80 | Ac-QS$/r5QTFSt//NLW$/LLAibQN-NH₂ | 820 | 2088.18 | 1045.09 | 1047.10 |
| 81 | Ac-QSQQTFSNLWRLLAibQN-NH₂ | 821 | 1988.02 | 995.01 | 995.31 |
| 82 | Hep/QSQ$/TF$/r8NLWRLL$/QN-NH₂ | 822 | 2215.29 | 1108.65 | 1108.93 |
| 83 | Ac-ASQQTF$r8NLRWLL$QN-NH₂ | 823 | 2051.13 | 1026.57 | 1026.90 |
| 84 | Ac-QSQQTF$/r8NLWRLLS/Q-NH₂ | 824 | 2022.14 | 1012.07 | 1012.66 |
| 85 | Ac-QSQQTF$r8NLWRLL$Q-NH₂ | 825 | 1994.11 | 998.06 | 998.42 |
| 86 | Ac-AAARAA$r8AAARAA$AA-NH₂ | 826 | 1515.90 | 758.95 | 759.21 |
| 87 | Ac-LTFEHYWAQLTSA-NH₂ | 827 | 1606.78 | 804.39 | 804.59 |
| 88 | Ac-LTF$r8HYWAQL$SA-NH₂ | 828 | 1668.90 | 835.45 | 835.67 |
| 89 | Ac-ASQQTFSNLWRLLPQN-NH₂ | 829 | 1943.00 | 972.50 | 971.27 |
| 90 | Ac-QS$QTFStNLW$r5LLAibQN-NH₂ | 830 | 2032.12 | 1017.06 | 1017.30 |
| 91 | Ac-QSQQTFAibNLWRLLAibQN-NH₂ | 831 | 1986.04 | 994.02 | 994.19 |
| 92 | Ac-QSQQTFNleNLWRLLNleQN-NH₂ | 832 | 2042.11 | 1022.06 | 1022.23 |
| 93 | Ac-QSQQTF$/r8NLWRLLAibQN-NH₂ | 833 | 2082.14 | 1042.07 | 1042.23 |
| 94 | Ac-QSQQTF$/r8NLWRLLNleQN-NH₂ | 834 | 2110.17 | 1056.09 | 1056.29 |
| 95 | Ac-QSQQTFAibNLWRLLS/QN-NH₂ | 835 | 2040.09 | 1021.05 | 1021.25 |
| 96 | Ac-QSQQTFNleNLWRLL$/QN-NH₂ | 836 | 2068.12 | 1035.06 | 1035.31 |
| 97 | Ac-QSQQTF%r8NL6clWRNleL%QN-NH₂ | 837 | 2144.13 | 1073.07 | 1071.32 |
| 98 | Ac-QSQQTF%r8NLMe6clWRLL%QN-NH₂ | 838 | 2158.15 | 1080.08 | 1080.31 |
| 101 | Ac-FNle$YWE$L-NH₂ | 839 | 1160.63 | — | 1161.70 |
| 102 | Ac-F$r8AYWELL$A-NH₂ | 840 | 1344.75 | — | 1345.90 |
| 103 | Ac-F$r8AYWQLL$A-NH₂ | 841 | 1341.76 | — | 1344.83 |
| 104 | Ac-NlePRF$r8NYWELL$QN-NH₂ | 842 | 1925.06 | 96153 | 961.69 |
| 105 | Ac-NlePRF$r8DYWRLL$QN-NH₂ | 843 | 1953.10 | 977.55 | 977.68 |
| 106 | Ac-NlePRFSr8NYWRLLSQ-NH₂ | 844 | 1838.07 | 920.04 | 920.18 |
| 107 | Ac-NlePRF$r8NYWRLL$-NH₂ | 845 | 1710.01 | 856.01 | 856.13 |
| 108 | Ac-QSQQTFSr8DLWRLLSQN-NH₂ | 846 | 2109.14 | 1055.57 | 1055.64 |
| 109 | Ac-QSQQTFSr8NLWRLLSEN-NH₂ | 847 | 2109.14 | 1055.57 | 1055.70 |
| 110 | Ac-QSQQTFSr8NLWRLLSQD-NH₂ | 848 | 2109.14 | 1055.57 | 1055.64 |
| 111 | Ac-QSQQTF$r8NLWRLL$S-NH₂ | 849 | 1953.08 | 977.54 | 977.60 |
| 112 | Ac-ESQQTFSr8NLWRLLSQN-NH₂ | 850 | 2109.14 | 1055.57 | 1055.70 |
| 113 | Ac-LTFSr8NLWRNleLSQ-NH₂ | 851 | 1635.99 | 819.00 | 819.10 |
| 114 | Ac-LRFSr8NLWRNleLSQ-NH₂ | 852 | 1691.04 | 846.52 | 846.68 |
| 115 | Ac-QSQQTFSr8NWWRNleLSQN-NH₂ | 853 | 2181.15 | 1091.58 | 1091.64 |
| 116 | Ac-QSQQTFSr8NLWRNleLSQ-NH₂ | 854 | 1994.11 | 998.06 | 998.07 |
| 117 | Ac-QTFSr8NLWRNleLSQN-NH₂ | 855 | 1765.00 | 883.50 | 883.59 |
| 118 | Ac-NlePRFSr8NWWRLLSQN-NH₂ | 856 | 1975.13 | 988.57 | 988.75 |
| 119 | Ac-NlePRFSr8NWWRLLSA-NH₂ | 857 | 1804.07 | 903.04 | 903.08 |
| 120 | Ac-TSFAEYWNLLNH₂ | 858 | 1467.70 | 734.85 | 734.90 |
| 121 | Ac-QTF$r8HWWSQL$S-NH₂ | 859 | 1651.85 | 826.93 | 827.12 |
| 122 | Ac-FM$YWE$L-NH₂ | 860 | 1178.58 | — | 1179.64 |
| 123 | Ac-QTFEHWWSQLLS-NH₂ | 861 | 1601.76 | 801.88 | 801.94 |
| 124 | Ac-QSQQTFSr8NLAmwRLNleSQN-NH₂ | 862 | 2122.17 | 1062.09 | 1062.24 |
| 125 | Ac-FMAibY6clWEAc3cL-NH₂ | 863 | 1130.47 | — | 1131.53 |
| 126 | Ac-FNle$Y6clWE$L-NH₂ | 864 | 1194.59 | — | 1195.64 |
| 127 | Ac-FSzr8AY6clWEAc3cLSz-NH₂ | 865 | 1277.63 | 639.82 | 1278.71 |
| 128 | Ac-FSr8AY6clWEAc3cLSA-NH₂ | 866 | 1348.66 | — | 1350.72 |
| 129 | Ac-NlePRFSr8NY6clWRLLSQN-NH₂ | 867 | 1986.08 | 994.04 | 994.64 |
| 130 | Ac-AF$r8AAWALA$A-NH₂ | 868 | 1223.71 | — | 1224.71 |
| 131 | Ac-TFSr8AAWRLASQ-NH₂ | 869 | 1395.80 | 698.90 | 399.04 |
| 132 | Pr-TFSr8AAWRLASQ-NH₂ | 870 | 1409.82 | 705.91 | 706.04 |
| 133 | Ac-QSQQTF%r8NLWRNleL%QN-NH₂ | 871 | 2110.17 | 1056.09 | 1056.22 |
| 134 | Ac-LTF%r8HYwAQL%sA-NH₂ | 872 | 1670.92 | 836.46 | 836.58 |
| 135 | Ac-NlePRF%r8NYWRLL%QN-NH₂ | 873 | 1954.13 | 978.07 | 978.19 |
| 136 | Ac-NlePRF%r8NY6clWRLL%QN-NH₂ | 874 | 1988.09 | 995.05 | 995.68 |
| 137 | Ac-LTF%r8HY6clWAQL%S-NH₂ | 875 | 1633.84 | 817.92 | 817.93 |
| 138 | Ac-QS%QTF%StNLWRLL%s8QN-NH₂ | 876 | 2149.24 | 1075.62 | 1075.65 |
| 139 | Ac-LTF%r8HY6clWRQL%S-NH₂ | 877 | 1718.91 | 860.46 | 860.54 |
| 140 | Ac-QSQQTF%r8NL6clWRLL%QN-NH₂ | 878 | 2144.13 | 1073.07 | 1073.64 |
| 141 | Ac-%r8SQQTFS%LWRLLAibQN-NH₂ | 879 | 2040.15 | 1021.08 | 1021.13 |
| 142 | Ac-LTF%r8HYWAQL%S-NH₂ | 880 | 1599.88 | 800.94 | 801.09 |
| 143 | Ac-TSF%r8QYWNLL%P-NH₂ | 881 | 1602.88 | 802.44 | 802.58 |
| 147 | Ac-LTFEHYWAQLTS-NH₂ | 882 | 1535.74 | 768.87 | 769.5 |
| 152 | Ac-F$er8AY6clWEAc3cL$e-NH₂ | 883 | 1277.63 | 639.82 | 1278.71 |
| 153 | Ac-AFSr8AAWALASA-NH₂ | 884 | 1277.63 | 639.82 | 1277.84 |
| 154 | Ac-TF$r8AAWRLA$Q-NH₂ | 885 | 1395.80 | 698.90 | 699.04 |
| 155 | Pr-TF$r8AAWRLA$Q-NH₂ | 886 | 1409.82 | 705.91 | 706.04 |
| 156 | Ac-LTF$er8HYWAQL$eS-NH₂ | 887 | 1597.87 | 799.94 | 800.44 |
| 159 | Ac-CCPGCCBaQSQQTF$r8NLWRLL$QN-NH₂ | 888 | 2745.30 | 1373.65 | 1372.99 |
| 160 | Ac-CCPGCCBaQSQQTA$r8NLWRLL$QN-NH₂ | 889 | 2669.27 | 1335.64 | 1336.09 |
| 161 | Ac-CCPGCCBaNlePRF$r8NYWRLL$QN-NH₂ | 890 | 2589.26 | 1295.63 | 1296.2 |
| 162 | Ac-LTF$/r8HYWAQL$/S-NH₂ | 891 | 1625.90 | 813.95 | 814.18 |
| 163 | Ac-F%r8HY6clWRAc3cL%-NH₂ | 892 | 1372.72 | 687.36 | 687.59 |
| 164 | Ac-QTF%r8HWWSQL%S-NH₂ | 893 | 1653.87 | 827.94 | 827.94 |
| 165 | Ac-LTA$r8HYWRQL$S-NH₂ | 894 | 1606.90 | 804.45 | 804.66 |
| 166 | Ac-Q$r8QQTFSN$WRLLAibQN-NH₂ | 895 | 2080.12 | 1041.06 | 1041.61 |
| 167 | Ac-QSQQ$r8FSNLWR$LAibQN-NH₂ | 896 | 2066.11 | 1034.06 | 1034.58 |
| 168 | Ac-F$r8AYWEAc3cL$A-NH₂ | 897 | 1314.70 | 658.35 | 1315.88 |
| 169 | Ac-F$r8AYWEAc3cL$S-NH₂ | 898 | 1330.70 | 666.35 | 1331.87 |
| 170 | Ac-F$r8AYWEAc3cL$Q-NH₂ | 899 | 1371.72 | 686.86 | 1372.72 |
| 171 | Ac-F$r8AYWEAibL$S-NH₂ | 900 | 1332.71 | 667.36 | 1334.83 |
| 172 | Ac-F$r8AYWEAL$S-NH₂ | 901 | 1318.70 | 660.35 | 1319.73 |
| 173 | Ac-F$r8AYWEQL$S-NH₂ | 902 | 1375.72 | 688.86 | 1377.53 |
| 174 | Ac-F$r8HYWEQL$S-NH₂ | 903 | 1441.74 | 721.87 | 1443.48 |
| 175 | Ac-F$r8HYWAQL$S-NH₂ | 904 | 1383.73 | 692.87 | 1385.38 |
| 176 | Ac-F$r8HYWAAc3cL$S-NH₂ | 905 | 1338.71 | 670.36 | 1340.82 |
| 177 | Ac-F$r8HYWRAc3cL$S-NH₂ | 906 | 1423.78 | 712.89 | 713.04 |
| 178 | Ac-F$r8AYWEAc3cL#A-NH₂ | 907 | 1300.69 | 651.35 | 1302.78 |
| 179 | Ac-NlePTF%r8NYWRLL%QN-NH₂ | 908 | 1899.08 | 950.54 | 950.56 |
| 180 | Ac-TF$r8AAWRAL$Q-NH₂ | 909 | 1395.80 | 698.90 | 699.13 |
| 181 | Ac-TSF%r8HYWAQL%S-NH₂ | 910 | 1573.83 | 787.92 | 787.98 |
| 184 | Ac-F%r8AY6clWEAc3cL%A-NH₂ | 911 | 1350.68 | 676.34 | 676.91 |
| 185 | Ac-LTF$r8HYWAQI$S-NH₂ | 912 | 1597.87 | 799.94 | 800.07 |
| 186 | Ac-LTF$r8HYWAQNle$S-NH₂ | 913 | 1597.87 | 799.94 | 800.07 |
| 187 | Ac-LTF$r8HYWAQL$A-NH₂ | 914 | 1581.87 | 791.94 | 792.45 |
| 188 | Ac-LTF$r8HYWAQL$Abu-NH₂ | 915 | 1595.89 | 798.95 | 799.03 |
| 189 | Ac-LTF$r8HYWAbuQL$S-NH₂ | 916 | 1611.88 | 806.94 | 807.47 |
| 190 | Ac-LTF$er8AYWAQL$eS-NH₂ | 917 | 1531.84 | 766.92 | 766.96 |
| 191 | Ac-LAF$r8HYWAQL$S-NH₂ | 918 | 1567.86 | 784.93 | 785.49 |
| 192 | Ac-LAF$r8AYWAQL$S-NH₂ | 919 | 1501.83 | 751.92 | 752.01 |
| 193 | Ac-LTF$er8AYWAQL$eA-NH₂ | 920 | 1515.85 | 758.93 | 758.97 |
| 194 | Ac-LAF$r8AYWAQL$A-NH₂ | 921 | 1485.84 | 743.92 | 744.05 |
| 195 | Ac-LTF$r8NLWANleL$Q-NH₂ | 922 | 1550.92 | 776.46 | 776.61 |
| 196 | Ac-LTF$r8NLWANleL$A-NH₂ | 923 | 1493.90 | 747.95 | 1495.6 |
| 197 | Ac-LTF$r8ALWANleL$Q-NH₂ | 924 | 1507.92 | 754.96 | 755 |
| 198 | Ac-LAF$r8NLWANleL$Q-NH₂ | 925 | 1520.91 | 761.46 | 761.96 |
| 199 | Ac-LAF$r8ALWANleL$A-NH₂ | 926 | 1420.89 | 711.45 | 1421.74 |
| 200 | Ac-A$r8AYwEAc3cL$A-NH₂ | 927 | 1238.67 | 620.34 | 1239.65 |
| 201 | Ac-F$r8AYWEAc3cL$AA-NH₂ | 928 | 1385.74 | 693.87 | 1386.64 |
| 202 | Ac-F$r8AYWEAc3cL$Abu-NH₂ | 929 | 1328.72 | 665.36 | 1330.17 |
| 203 | Ac-F$r8AYWEAc3cL$Nle-NH₂ | 930 | 1356.75 | 679.38 | 1358.22 |
| 204 | Ac-F$r5AYWEAc3cL$s8A-NH₂ | 931 | 1314.70 | 658.35 | 1315.51 |
| 205 | Ac-F$AYWEAc3cL$r8A-NH₂ | 932 | 1314.70 | 658.35 | 1315.66 |
| 206 | Ac-F$r8AYWEAc3cI$A-NH₂ | 933 | 1314.70 | 658.35 | 1316.18 |
| 207 | Ac-F$r8AYWEAc3cNle$A-NH₂ | 934 | 1314.70 | 658.35 | 1315.66 |
| 208 | Ac-F$r8AYWEAmlL$A-NH₂ | 935 | 1358.76 | 680.38 | 1360.21 |
| 209 | Ac-F$r8AYWENleL$A-NH₂ | 936 | 1344.75 | 673.38 | 1345.71 |
| 210 | Ac-F$r8AYWQAc3cL$A-NH₂ | 937 | 1313.72 | 657.86 | 1314.7 |
| 211 | Ac-F$r8AYwAAc3cL$A-NH₂ | 938 | 1256.70 | 629.35 | 1257.56 |
| 212 | Ac-F$r8AYWAbuAc3cL$A-NH₂ | 939 | 1270.71 | 636.36 | 1272.14 |
| 213 | Ac-F$r8AYWNleAc3cL$A-NH₂ | 940 | 1298.74 | 650.37 | 1299.67 |
| 214 | Ac-F$r8AbuYWEAc3cL$A-NH₂ | 941 | 1328.72 | 665.36 | 1329.65 |
| 215 | Ac-F$r8NleYWEAc3cL$A-NH₂ | 942 | 1356.75 | 679.38 | 1358.66 |
| 216 | 5-FAM-BaLTFEHYWAQLTS-NH₂ | 943 | 1922.82 | 962.41 | 962.87 |
| 217 | 5-FAM-BaLTF%r8HYWAQL%S-NH₂ | 944 | 1986.96 | 994.48 | 994.97 |
| 218 | Ac-LTF$r8HYWAQhL$S-NH₂ | 945 | 1611.88 | 806.94 | 807 |
| 219 | Ac-LTF$r8HYWAQTle$S-NH₂ | 946 | 1597.87 | 799.94 | 799.97 |
| 220 | Ac-LTF$r8HYWAQAdm$S-NH₂ | 947 | 1675.91 | 838.96 | 839.09 |
| 221 | Ac-LTF$r8HYWAQhCha$S-NH₂ | 948 | 1651.91 | 826.96 | 826.98 |
| 222 | Ac-LTF$r8HYWAQCha$S-NH₂ | 949 | 1637.90 | 819.95 | 820.02 |
| 223 | Ac-LTF$r8HYWAc6cQL$S-NH₂ | 950 | 1651.91 | 826.96 | 826.98 |
| 224 | Ac-LTF$r8HYWAc5cQL$S-NH₂ | 951 | 1637.90 | 819.95 | 820.02 |
| 225 | Ac-LThF$r8HYWAQL$S-NH₂ | 952 | 1611.88 | 806.94 | 807 |
| 226 | Ac-LTIg1$r8HYWAQL$S-NH₂ | 953 | 1625.90 | 813.95 | 812.99 |
| 227 | Ac-LTF$r8HYwAQChg-S S-NH₂ | 954 | 1623.88 | 812.94 | 812.99 |
| 228 | Ac-LTF$r8HYWAQF$S-NH₂ | 955 | 1631.85 | 816.93 | 816.99 |
| 229 | Ac-LTF$r8HYWAQIgl$S-NH₂ | 956 | 1659.88 | 830.94 | 829.94 |
| 230 | Ac-LTF$r8HYWAQCba$S-NH₂ | 957 | 1609.87 | 805.94 | 805.96 |
| 231 | Ac-LTF$r8HYWAQCpg$S-NH₂ | 958 | 1609.87 | 805.94 | 805.96 |
| 232 | Ac-LTF$r8HhYWAQL$S-NH₂ | 959 | 1611.88 | 806.94 | 807 |
| 233 | Ac-F$r8AYWEAc3chL$A-NH₂ | 960 | 1328.72 | 665.36 | 665.43 |
| 234 | Ac-F$r8AYWEAc3cTle$A-NH₂ | 961 | 1314.70 | 658.35 | 1315.62 |
| 235 | Ac-F$r8AYWEAc3cAdm$A-NH₂ | 962 | 1392.75 | 697.38 | 697.47 |
| 236 | Ac-F$r8AYWEAc3chCha$A-NH₂ | 963 | 1368.75 | 685.38 | 685.34 |
| 237 | Ac-F$r8AYWEAc3cCha$A-NH₂ | 964 | 1354.73 | 678.37 | 678.38 |
| 238 | Ac-F$r8AYWEAc6cL$A-NH₂ | 965 | 1356.75 | 679.38 | 679.42 |
| 239 | Ac-F$r8AYWEAc5cL$A-NH₂ | 966 | 1342.73 | 672.37 | 672.46 |
| 240 | Ac-hF$r8AYWEAc3cL$A-NH₂ | 967 | 1328.72 | 665.36 | 665.43 |
| 241 | Ac-Ig1$r8AYWEAc3cL$A-NH₂ | 968 | 1342.73 | 672.37 | 671.5 |
| 243 | Ac-F$r8AYWEAc3cF$A-NH₂ | 969 | 1348.69 | 675.35 | 675.35 |
| 244 | Ac-F$r8AYWEAc3cIg1$A-NH₂ | 970 | 1376.72 | 689.36 | 688.37 |
| 245 | Ac-F$r8AYWEAc3cCba$A-NH₂ | 971 | 1326.70 | 664.35 | 664.47 |
| 246 | Ac-F$r8AYWEAc3cCpg$A-NH₂ | 972 | 1326.70 | 664.35 | 664.39 |
| 247 | Ac-F$r8AhYWEAc3cL$A-NH₂ | 973 | 1328.72 | 665.36 | 665.43 |
| 248 | Ac-F$r8AYWEAc3cL$Q-NH₂ | 974 | 1371.72 | 686.86 | 1372.87 |
| 249 | Ac-F$r8AYWEAibL$A-NH₂ | 975 | 1316.72 | 659.36 | 1318.18 |
| 250 | Ac-F$r8AYWEAL$A-NH₂ | 976 | 1302.70 | 652.35 | 1303.75 |
| 251 | Ac-LAF$r8AYWAAL$A-NH₂ | 977 | 1428.82 | 715.41 | 715.49 |
| 252 | Ac-LTF$r8HYWAAc3cL$S-NH₂ | 978 | 1552.84 | 777.42 | 777.5 |
| 253 | Ac-NleTF$r8HYWAQL$S-NH₂ | 979 | 1597.87 | 799.94 | 800.04 |
| 254 | Ac-VTF$r8HYWAQL$S-NH₂ | 980 | 1583.85 | 792.93 | 793.04 |
| 255 | Ac-FTF$r8HYWAQL$S-NH₂ | 981 | 1631.85 | 816.93 | 817.02 |
| 256 | Ac-WTF$r8HYWAQL$S-NH₂ | 982 | 1670.86 | 836.43 | 836.85 |
| 257 | Ac-RTF$r8HYWAQL$S-NH₂ | 983 | 1640.88 | 821.44 | 821.9 |
| 258 | Ac-KTF$r8HYWAQL$S-NH₂ | 984 | 1612.88 | 807.44 | 807.91 |
| 259 | Ac-LNleF$r8HYWAQL$S-NH₂ | 985 | 1609.90 | 805.95 | 806.43 |
| 260 | Ac-LVF$r8HYWAQL$S-NH₂ | 986 | 1595.89 | 798.95 | 798.93 |
| 261 | Ac-LFF$r8HYWAQL$S-NH₂ | 987 | 1643.89 | 822.95 | 823.38 |
| 262 | Ac-LWF$r8HYWAQL$S-NH₂ | 988 | 1682.90 | 842.45 | 842.55 |
| 263 | Ac-LRF$r8HYWAQL$S-NH₂ | 989 | 1652.92 | 827.46 | 827.52 |
| 264 | Ac-LKF$r8HYWAQL$S-NH₂ | 990 | 1624.91 | 813.46 | 813.51 |
| 265 | Ac-LTF$r8NleYWAQL$S-NH₂ | 991 | 1573.89 | 787.95 | 788.05 |
| 266 | Ac-LTF$r8VYWAQL$S-NH₂ | 992 | 1559.88 | 780.94 | 780.98 |
| 267 | Ac-LTF$r8FYWAQL$S-NH₂ | 993 | 1607.88 | 804.94 | 805.32 |
| 268 | Ac-LTF$r8WYWAQL$S-NH₂ | 994 | 1646.89 | 824.45 | 824.86 |
| 269 | Ac-LTF$r8RYWAQL$S-NH₂ | 995 | 1616.91 | 809.46 | 809.51 |
| 270 | Ac-LTF$r8KYWAQL$S-NH₂ | 996 | 1588.90 | 795.45 | 795.48 |
| 271 | Ac-LTF$r8HNleWAQL$S-NH₂ | 997 | 1547.89 | 774.95 | 774.98 |
| 272 | Ac-LTF$r8HVWAQL$S-NH₂ | 998 | 1533.87 | 767.94 | 767.95 |
| 273 | Ac-LTF$r8HFWAQL$S-NH₂ | 999 | 1581.87 | 791.94 | 792.3 |
| 274 | Ac-LTF$r8HWWAQL$S-NH₂ | 1000 | 1620.88 | 811.44 | 811.54 |
| 275 | Ac-LTF$r8HRWAQL$S-NH₂ | 1001 | 1590.90 | 796.45 | 796.52 |
| 276 | Ac-LTF$r8HKWAQL$S-NH₂ | 1002 | 1562.90 | 782.45 | 782.53 |
| 277 | Ac-LTF$r8HYWNleQL$S-NH₂ | 1003 | 1639.91 | 820.96 | 820.98 |
| 278 | Ac-LTF$r8HYWVQL$S-NH₂ | 1004 | 1625.90 | 813.95 | 814.03 |
| 279 | Ac-LTF$r8HYWFQL$S-NH₂ | 1005 | 1673.90 | 837.95 | 838.03 |
| 280 | Ac-LTF$r8HYWWQL$S-NH₂ | 1006 | 1712.91 | 857.46 | 857.5 |
| 281 | Ac-LTF$r8HYWKQL$S-NH₂ | 1007 | 1654.92 | 828.46 | 828.49 |
| 282 | Ac-LTF$r8HYWANleL$S-NH₂ | 1008 | 1582.89 | 792.45 | 792.52 |
| 283 | Ac-LTF$r8HYWAVL$S-NH₂ | 1009 | 1568.88 | 785.44 | 785.49 |
| 284 | Ac-LTF$r8HYWAFL$S-NH₂ | 1010 | 1616.88 | 809.44 | 809.47 |
| 285 | Ac-LTF$r8HYWAWL$S-NH₂ | 1011 | 1655.89 | 828.95 | 829 |
| 286 | Ac-LTF$r8HYWARL$S-NH₂ | 1012 | 1625.91 | 813.96 | 813.98 |
| 287 | Ac-LTF$r8HYWAQL$Nle-NH₂ | 1013 | 1623.92 | 812.96 | 813.39 |
| 288 | Ac-LTF$r8HYWAQL$V-NH₂ | 1014 | 1609.90 | 805.95 | 805.99 |
| 289 | Ac-LTF$r8HYWAQL$F-NH₂ | 1015 | 1657.90 | 829.95 | 830.26 |
| 290 | Ac-LTF$r8HYWAQL$W-NH₂ | 1016 | 1696.91 | 849.46 | 849.5 |
| 291 | Ac-LTF$r8HYWAQL$R-NH₂ | 1017 | 1666.94 | 834.47 | 834.56 |
| 292 | Ac-LTF$r8HYWAQL$K-NH₂ | 1018 | 1638.93 | 820.47 | 820.49 |
| 293 | Ac-Q$r8QQTFSN$wRLLAibQN-NH₂ | 1019 | 2080.12 | 1041.06 | 1041.54 |
| 294 | Ac-QSQQ$r8FSNLWR$LAibQN-NH₂ | 1020 | 2066.11 | 1034.06 | 1034.58 |
| 295 | Ac-LT2Pal$r8HYWAQL$S-NH₂ | 1021 | 1598.86 | 800.43 | 800.49 |
| 296 | Ac-LT3Pal$r8HYWAQL$S-NH₂ | 1022 | 1598.86 | 800.43 | 800.49 |
| 297 | Ac-LT4Pal$r8HYWAQL$S-NH₂ | 1023 | 1598.86 | 800.43 | 800.49 |
| 298 | Ac-LTF2CF3$r8HYWAQL$S-NH₂ | 1024 | 1665.85 | 833.93 | 834.01 |
| 299 | Ac-LTF2CN$r8HYWAQL$S-NH₂ | 1025 | 1622.86 | 812.43 | 812.47 |
| 300 | Ac-LTF2Me$r8HYWAQL$S-NH₂ | 1026 | 1611.88 | 806.94 | 807 |
| 301 | Ac-LTF3Cl$r8HYWAQL$S-NH₂ | 1027 | 1631.83 | 816.92 | 816.99 |
| 302 | Ac-LTF4CF3$r8HYwAQL$S-NH₂ | 1028 | 1665.85 | 833.93 | 833.94 |
| 303 | Ac-LTF4tBu$r8HYWAQL$S-NH₂ | 1029 | 1653.93 | 827.97 | 828.02 |
| 304 | Ac-LTF5F$r8HYWAQL$S-NH₂ | 1030 | 1687.82 | 844.91 | 844.96 |
| 305 | Ac-LTF$r8HY3BthAAQL$S-NH₂ | 1031 | 1614.83 | 808.42 | 808.48 |
| 306 | Ac-LTF2Br$r8HYWAQL$S-NH₂ | 1032 | 1675.78 | 838.89 | 838.97 |
| 307 | Ac-LTF4Br$r8HYWAQL$S-NH₂ | 1033 | 1675.78 | 838.89 | 839.86 |
| 308 | Ac-LTF2Cl$r8HYWAQL$S-NH₂ | 1034 | 1631.83 | 816.92 | 816.99 |
| 309 | Ac-LTF4Cl$r8HYWAQL$S-NH₂ | 1035 | 1631.83 | 816.92 | 817.36 |
| 310 | Ac-LTF3CN$r8HYWAQL$S-NH₂ | 1036 | 1622.86 | 812.43 | 812.47 |
| 311 | Ac-LTF4CN$r8HYWAQL$S-NH₂ | 1037 | 1622.86 | 812.43 | 812.47 |
| 312 | Ac-LTF34Cl2$r8HYWAQL$S-NH₂ | 1038 | 1665.79 | 833.90 | 833.94 |
| 313 | Ac-LTF34F2$r8HYWAQL$S-NH₂ | 1039 | 1633.85 | 817.93 | 817.95 |
| 314 | Ac-LTF35F2$r8HYWAQL$S-NH₂ | 1040 | 1633.85 | 817.93 | 817.95 |
| 315 | Ac-LTDip$r8HYWAQL$S-NH₂ | 1041 | 1673.90 | 837.95 | 838.01 |
| 316 | Ac-LTF2F$r8HYWAQL$S-NH₂ | 1042 | 1615.86 | 808.93 | 809 |
| 317 | Ac-LTF3F$r8HYWAQL$S-NH₂ | 1043 | 1615.86 | 808.93 | 809 |
| 318 | Ac-LTF4F$r8HYWAQL$S-NH₂ | 1044 | 1615.86 | 808.93 | 809 |
| 319 | Ac-LTF4I$r8HYWAQL$S-NH₂ | 1045 | 1723.76 | 862.88 | 862.94 |
| 320 | Ac-LTF3Me$r8HYWAQL$S-NH₂ | 1046 | 1611.88 | 806.94 | 807.07 |
| 321 | Ac-LTF4Me$r8HYWAQL$S-NH₂ | 1047 | 1611.88 | 806.94 | 807 |
| 322 | Ac-LT1Nal$r8HYWAQL$S-NH₂ | 1048 | 1647.88 | 824.94 | 824.98 |
| 323 | Ac-LT2Nal$r8HYWAQL$S-NH₂ | 1049 | 1647.88 | 824.94 | 825.06 |
| 324 | Ac-LTF3CF3$r8HYWAQL$S-NH₂ | 1050 | 1665.85 | 833.93 | 834.01 |
| 325 | Ac-LTF4NO2$r8HYWAQL$S-NH₂ | 1051 | 1642.85 | 822.43 | 822.46 |
| 326 | Ac-LTF3NO2$r8HYWAQL$S-NH₂ | 1052 | 1642.85 | 822.43 | 822.46 |
| 327 | Ac-LTF$r82ThiYWAQL$S-NH₂ | 1053 | 1613.83 | 807.92 | 807.96 |
| 328 | Ac-LTF$r8HBipWAQL$S-NH₂ | 1054 | 1657.90 | 829.95 | 830.01 |
| 329 | Ac-LTF$r8HF4tBuWAQL$S-NH₂ | 1055 | 1637.93 | 819.97 | 820.02 |
| 330 | Ac-LTF$r8HF4CF3WAQL$S-NH₂ | 1056 | 1649.86 | 825.93 | 826.02 |
| 331 | Ac-LTF$r8HF4C1WAQL$S-NH₂ | 1057 | 1615.83 | 808.92 | 809.37 |
| 332 | Ac-LTF$r8HF4MeWAQL$S-NH₂ | 1058 | 1595.89 | 798.95 | 799.01 |
| 333 | Ac-LTF$r8HF4BrWAQL$S-NH₂ | 1059 | 1659.78 | 830.89 | 830.98 |
| 334 | Ac-LTF$r8HF4CNWAQL$S-NH₂ | 1060 | 1606.87 | 804.44 | 804.56 |
| 335 | Ac-LTF$r8HF4NO2WAQL$S-NH₂ | 1061 | 1626.86 | 814.43 | 814.55 |
| 336 | Ac-LTF$r8H1NalWAQL$S-NH₂ | 1062 | 1631.89 | 816.95 | 817.06 |
| 337 | Ac-LTF$r8H2NalWAQL$S-NH₂ | 1063 | 1631.89 | 816.95 | 816.99 |
| 338 | Ac-LTF$r8HWAQL$S-NH₂ | 1064 | 1434.80 | 718.40 | 718.49 |
| 339 | Ac-LTF$r8HY1NalAQL$S-NH₂ | 1065 | 1608.87 | 805.44 | 805.52 |
| 340 | Ac-LTF$r8HY2NalAQL$S-NH₂ | 1066 | 1608.87 | 805.44 | 805.52 |
| 341 | Ac-LTF$r8HYWAQI$S-NH₂ | 1067 | 1597.87 | 799.94 | 800.07 |
| 342 | Ac-LTF$r8HYWAQNle$S-NH₂ | 1068 | 1597.87 | 799.94 | 800.44 |
| 343 | Ac-LTF$er8HYWAQL$eA-NH₂ | 1069 | 1581.87 | 791.94 | 791.98 |
| 344 | Ac-LTF$r8HYWAQL$Abu-NH₂ | 1070 | 1595.89 | 798.95 | 799.03 |
| 345 | Ac-LTF$r8HYWAbuQL$S-NH₂ | 1071 | 1611.88 | 806.94 | 804.47 |
| 346 | Ac-LAF$r8HYWAQL$S-NH₂ | 1072 | 1567.86 | 784.93 | 785.49 |
| 347 | Ac-LTF$r8NLWANleL$Q-NH₂ | 1073 | 1550.92 | 776.46 | 777.5 |
| 348 | Ac-LTF$r8ALWANleL$Q-NH₂ | 1074 | 1507.92 | 754.96 | 755.52 |
| 349 | Ac-LAF$r8NLWANleL$Q-NH₂ | 1075 | 1520.91 | 761.46 | 762.48 |
| 350 | Ac-F$r8AYWAAc3cL$A-NH₂ | 1076 | 1256.70 | 629.35 | 1257.56 |
| 351 | Ac-LTF$r8AYWAAL$S-NH₂ | 1077 | 1474.82 | 738.41 | 738.55 |
| 352 | Ac-LVF$r8AYWAQL$S-NH₂ | 1078 | 1529.87 | 765.94 | 766 |
| 353 | Ac-LTF$r8AYWAbuQL$S-NH₂ | 1079 | 1545.86 | 773.93 | 773.92 |
| 354 | Ac-LTF$r8AYWNleQL$S-NH₂ | 1080 | 1573.89 | 787.95 | 788.17 |
| 355 | Ac-LTF$r8AbuYWAQL$S-NH₂ | 1081 | 1545.86 | 773.93 | 773.99 |
| 356 | Ac-LTF$r8AYWHQL$S-NH₂ | 1082 | 1597.87 | 799.94 | 799.97 |
| 357 | Ac-LTF$r8AYWKQL$S-NH₂ | 1083 | 1588.90 | 795.45 | 795.53 |
| 358 | Ac-LTF$r8AYWOQL$S-NH₂ | 1084 | 1574.89 | 788.45 | 788.5 |
| 359 | Ac-LTF$r8AYWRQL$S-NH₂ | 1085 | 1616.91 | 809.46 | 809.51 |
| 360 | Ac-LTF$r8AYWSQL$S-NH₂ | 1086 | 1547.84 | 774.92 | 774.96 |
| 361 | Ac-LTF$r8AYWRAL$S-NH₂ | 1087 | 1559.89 | 780.95 | 780.95 |
| 362 | Ac-LTF$r8AYwRQL$A-NH₂ | 1088 | 1600.91 | 801.46 | 801.52 |
| 363 | Ac-LTF$r8AYWRAL$A-NH₂ | 1089 | 1543.89 | 772.95 | 773.03 |
| 364 | Ac-LTF$r5HYWAQL$s8S-NH₂ | 1090 | 1597.87 | 799.94 | 799.97 |
| 365 | Ac-LTF$HYWAQL$r8S-NH₂ | 1091 | 1597.87 | 799.94 | 799.97 |
| 366 | Ac-LTF$r8HYWAAL$S-NH₂ | 1092 | 1540.84 | 771.42 | 771.48 |
| 367 | Ac-LTF$r8HYWAAbuL$S-NH₂ | 1093 | 1554.86 | 778.43 | 778.51 |
| 368 | Ac-LTF$r8HYWALL$S-NH₂ | 1094 | 1582.89 | 792.45 | 792.49 |
| 369 | Ac-F$r8AYWHAL$A-NH₂ | 1095 | 1310.72 | 656.36 | 656.4 |
| 370 | Ac-F$r8AYWAAL$A-NH₂ | 1096 | 1244.70 | 623.35 | 1245.61 |
| 371 | Ac-F$r8AYWSAL$A-NH₂ | 1097 | 1260.69 | 631.35 | 1261.6 |
| 372 | Ac-F$r8AYWRAL$A-NH₂ | 1098 | 1329.76 | 665.88 | 1330.72 |
| 373 | Ac-F$r8AYWKAL$A-NH₂ | 1099 | 1301.75 | 651.88 | 1302.67 |
| 374 | Ac-F$r8AYWOAL$A-NH₂ | 1100 | 1287.74 | 644.87 | 1289.13 |
| 375 | Ac-F$r8VYWEAc3cL$A-NH₂ | 1101 | 1342.73 | 672.37 | 1343.67 |
| 376 | Ac-F$r8FYWEAc3cL$A-NH₂ | 1102 | 1390.73 | 696.37 | 1392.14 |
| 377 | Ac-F$r8WYWEAc3cL$A-NH₂ | 1103 | 1429.74 | 715.87 | 1431.44 |
| 378 | Ac-F$r8RYWEAc3cL$A-NH₂ | 1104 | 1399.77 | 700.89 | 700.95 |
| 379 | Ac-F$r8KYWEAc3cL$A-NH₂ | 1105 | 1371.76 | 686.88 | 686.97 |
| 380 | Ac-F$r8ANleWEAc3cL$A-NH₂ | 1106 | 1264.72 | 633.36 | 1265.59 |
| 381 | Ac-F$r8AVWEAc3cL$A-NH₂ | 1107 | 1250.71 | 626.36 | 1252.2 |
| 382 | Ac-F$r8AFWEAc3cL$A-NH₂ | 1108 | 1298.71 | 650.36 | 1299.64 |
| 383 | Ac-F$r8AWWEAc3cL$A-NH₂ | 1109 | 1337.72 | 669.86 | 1338.64 |
| 384 | Ac-F$r8ARWEAc3cL$A-NH₂ | 1110 | 1307.74 | 654.87 | 655 |
| 385 | Ac-F$r8AKWEAc3cL$A-NH₂ | 1111 | 1279.73 | 640.87 | 641.01 |
| 386 | Ac-F$r8AYWVAc3cL$A-NH₂ | 1112 | 1284.73 | 643.37 | 643.38 |
| 387 | Ac-F$r8AYWFAc3cL$A-NH₂ | 1113 | 1332.73 | 667.37 | 667.43 |
| 388 | Ac-F$r8AYWWAc3cL$A-NH₂ | 1114 | 1371.74 | 686.87 | 686.97 |
| 389 | Ac-F$r8AYWRAc3cL$A-NH₂ | 1115 | 1341.76 | 671.88 | 671.94 |
| 390 | Ac-F$r8AYWKAc3cL$A-NH₂ | 1116 | 1313.75 | 657.88 | 657.88 |
| 391 | Ac-F$r8AYWEVL$A-NH₂ | 1117 | 1330.73 | 666.37 | 666.47 |
| 392 | Ac-F$r8AYWEFL$A-NH₂ | 1118 | 1378.73 | 690.37 | 690.44 |
| 393 | Ac-F$r8AYWEWL$A-NH₂ | 1119 | 1417.74 | 709.87 | 709.91 |
| 394 | Ac-F$r8AYWERL$A-NH₂ | 1120 | 1387.77 | 694.89 | 1388.66 |
| 395 | Ac-F$r8AYWEKL$A-NH₂ | 1121 | 1359.76 | 680.88 | 1361.21 |
| 396 | Ac-F$r8AYWEAc3cL$V-NH₂ | 1122 | 1342.73 | 672.37 | 1343.59 |
| 397 | Ac-F$r8AYWEAc3cL$F-NH₂ | 1123 | 1390.73 | 696.37 | 1392.58 |
| 398 | Ac-F$r8AYWEAc3cL$W-NH₂ | 1124 | 1429.74 | 715.87 | 1431.29 |
| 399 | Ac-F$r8AYWEAc3cL$R-NH₂ | 1125 | 1399.77 | 700.89 | 700.95 |
| 400 | Ac-F$r8AYWEAc3cL$K-NH₂ | 1126 | 1371.76 | 686.88 | 686.97 |
| 401 | Ac-F$r8AYWEAc3cL$AV-NH₂ | 1127 | 1413.77 | 707.89 | 707.91 |
| 402 | Ac-F$r8AYWEAc3cL$AF-NH₂ | 1128 | 1461.77 | 731.89 | 731.96 |
| 403 | Ac-F$r8AYWEAc3cL$Aw-NH₂ | 1129 | 1500.78 | 751.39 | 751.5 |
| 404 | Ac-F$r8AYWEAc3cL$AR-NH₂ | 1130 | 1470.80 | 736.40 | 736.47 |
| 405 | Ac-F$r8AYWEAc3cL$AK-NH₂ | 1131 | 1442.80 | 722.40 | 722.41 |
| 406 | Ac-F$r8AYWEAc3cL$AH-NH₂ | 1132 | 1451.76 | 726.88 | 726.93 |
| 407 | Ac-LTF2NO2$r8HYWAQL$S-NH₂ | 1133 | 1642.85 | 822.43 | 822.54 |
| 408 | Ac-LTA$r8HYAAQL$S-NH₂ | 1134 | 1406.79 | 704.40 | 704.5 |
| 409 | Ac-LTF$r8HYAAQL$S-NH₂ | 1135 | 1482.82 | 742.41 | 742.47 |
| 410 | Ac-QSQQTF$r8NLWALL$AN-NH₂ | 1136 | 1966.07 | 984.04 | 984.38 |
| 411 | Ac-QAibQQTF$r8NLWALL$AN-NH₂ | 1137 | 1964.09 | 983.05 | 983.42 |
| 412 | Ac-QAibQQTF$r8ALWALL$AN-NH₂ | 1138 | 1921.08 | 961.54 | 961.59 |
| 413 | Ac-AAAATF$r8AAWAAL$AA-NH₂ | 1139 | 1608.90 | 805.45 | 805.52 |
| 414 | Ac-F$r8AAWRAL$Q-NH₂ | 1140 | 1294.76 | 648.38 | 648.48 |
| 415 | Ac-TF$r8AAWAAL$Q-NH₂ | 1141 | 1310.74 | 656.37 | 1311.62 |
| 416 | Ac-TF$r8AAWRAL$A-NH₂ | 1142 | 1338.78 | 670.39 | 670.46 |
| 417 | Ac-VF$r8AAWRAL$Q-NH₂ | 1143 | 1393.82 | 697.91 | 697.99 |
| 418 | Ac-AF$r8AAWAAL$A-NH₂ | 1144 | 1223.71 | 612.86 | 1224.67 |
| 420 | Ac-TF$r8AAWKAL$Q-NH₂ | 1145 | 1367.80 | 684.90 | 684.97 |
| 421 | Ac-TF$r8AAWOAL$Q-NH₂ | 1146 | 1353.78 | 677.89 | 678.01 |
| 422 | Ac-TF$r8AAWSAL$Q-NH₂ | 1147 | 1326.73 | 664.37 | 664.47 |
| 423 | Ac-LTF$r8AAWRAL$Q-NH₂ | 1148 | 1508.89 | 755.45 | 755.49 |
| 424 | Ac-F$r8AYWAQL$A-NH₂ | 1149 | 1301.72 | 651.86 | 651.96 |
| 425 | Ac-F$r8AWWAAL$A-NH₂ | 1150 | 1267.71 | 634.86 | 634.87 |
| 426 | Ac-F$r8AWWAQL$A-NH₂ | 1151 | 1324.73 | 663.37 | 663.43 |
| 427 | Ac-F$r8AYWEAL$-NH₂ | 1152 | 1231.66 | 616.83 | 1232.93 |
| 428 | Ac-F$r8AYWAAL$-NH₂ | 1153 | 1173.66 | 587.83 | 1175.09 |
| 429 | Ac-F$r8AYWKAL$-NH₂ | 1154 | 1230.72 | 616.36 | 616.44 |
| 430 | Ac-F$r8AYWOAL$-NH₂ | 1155 | 1216.70 | 609.35 | 609.48 |
| 431 | Ac-F$r8AYWQAL$-NH₂ | 1156 | 1230.68 | 616.34 | 616.44 |
| 432 | Ac-F$r8AYWAQL$-NH₂ | 1157 | 1230.68 | 616.34 | 616.37 |
| 433 | Ac-F$r8HYWDQL$S-NH₂ | 1158 | 1427.72 | 714.86 | 714.86 |
| 434 | Ac-F$r8HFWEQL$S-NH₂ | 1159 | 1425.74 | 713.87 | 713.98 |
| 435 | Ac-F$r8AYWHQL$S-NH₂ | 1160 | 1383.73 | 692.87 | 692.96 |
| 436 | Ac-F$r8AYWKQL$S-NH₂ | 1161 | 1374.77 | 688.39 | 688.45 |
| 437 | Ac-F$r8AYWOQL$S-NH₂ | 1162 | 1360.75 | 681.38 | 681.49 |
| 438 | Ac-F$r8HYWSQL$S-NH₂ | 1163 | 1399.73 | 700.87 | 700.95 |
| 439 | Ac-F$r8HWWEQL$S-NH₂ | 1164 | 1464.76 | 733.38 | 733.44 |
| 440 | Ac-F$r8HWWAQL$S-NH₂ | 1165 | 1406.75 | 704.38 | 704.43 |
| 441 | Ac-F$r8AWWHQL$S-NH₂ | 1166 | 1406.75 | 704.38 | 704.43 |
| 442 | Ac-F$r8AWWKQL$S-NH₂ | 1167 | 1397.79 | 699.90 | 699.92 |
| 443 | Ac-F$r8AWWOQL$S-NH₂ | 1168 | 1383.77 | 692.89 | 692.96 |
| 444 | Ac-F$r8HWWSQL$S-NH₂ | 1169 | 1422.75 | 712.38 | 712.42 |
| 445 | Ac-LTF$r8NYWANleL$Q-NH₂ | 1170 | 1600.90 | 801.45 | 801.52 |
| 446 | Ac-LTF$r8NLWAQL$Q-NH₂ | 1171 | 1565.90 | 783.95 | 784.06 |
| 447 | Ac-LTF$r8NYWANleL$A-NH₂ | 1172 | 1543.88 | 772.94 | 773.03 |
| 448 | Ac-LTF$r8NLWAQL$A-NH₂ | 1173 | 1508.88 | 755.44 | 755.49 |
| 449 | Ac-LTF$r8AYWANleL$Q-NH₂ | 1174 | 1557.90 | 779.95 | 780.06 |
| 450 | Ac-LTF$r8ALWAQL$Q-NH₂ | 1175 | 1522.89 | 762.45 | 762.45 |
| 451 | Ac-LAF$r8NYWANleL$Q-NH₂ | 1176 | 1570.89 | 786.45 | 786.5 |
| 452 | Ac-LAF$r8NLWAQL$Q-NH₂ | 1177 | 1535.89 | 768.95 | 769.03 |
| 453 | Ac-LAF$r8AYWANleL$A-NH₂ | 1178 | 1470.86 | 736.43 | 736.47 |
| 454 | Ac-LAF$r8ALWAQL$A-NH₂ | 1179 | 1435.86 | 718.93 | 719.01 |
| 455 | Ac-LAF$r8AYWAAL$A-NH₂ | 1180 | 1428.82 | 715.41 | 715.41 |
| 456 | Ac-F$r8AYWEAc3cL$AAib-NH₂ | 1181 | 1399.75 | 700.88 | 700.95 |
| 457 | Ac-F$r8AYWAQL$AA-NH₂ | 1182 | 1372.75 | 687.38 | 687.78 |
| 458 | Ac-F$r8AYWAAc3cL$AA-NH₂ | 1183 | 1327.73 | 664.87 | 664.84 |
| 459 | Ac-F$r8AYWSAc3cL$AA-NH₂ | 1184 | 1343.73 | 672.87 | 672.9 |
| 460 | Ac-F$r8AYWEAc3cL$AS-NH₂ | 1185 | 1401.73 | 701.87 | 701.84 |
| 461 | Ac-F$r8AYWEAc3cL$AT-NH₂ | 1186 | 1415.75 | 708.88 | 708.87 |
| 462 | Ac-F$r8AYWEAc3cL$AL-NH₂ | 1187 | 1427.79 | 714.90 | 714.94 |
| 463 | Ac-F$r8AYWEAc3cL$AQ-NH₂ | 1188 | 1442.76 | 722.38 | 722.41 |
| 464 | Ac-F$r8AFWEAc3cL$AA-NH₂ | 1189 | 1369.74 | 685.87 | 685.93 |
| 465 | Ac-F$r8AWWEAc3cL$AA-NH₂ | 1190 | 1408.75 | 705.38 | 705.39 |
| 466 | Ac-F$r8AYWEAc3cL$SA-NH₂ | 1191 | 1401.73 | 701.87 | 701.99 |
| 467 | Ac-F$r8AYWEAL$AA-NH₂ | 1192 | 1373.74 | 687.87 | 687.93 |
| 468 | Ac-F$r8AYWENleL$AA-NH₂ | 1193 | 1415.79 | 708.90 | 708.94 |
| 469 | Ac-F$r8AYWEAc3cL$AbuA-NH₂ | 1194 | 1399.75 | 700.88 | 700.95 |
| 470 | Ac-F$r8AYWEAc3cL$NleA-NH₂ | 1195 | 1427.79 | 714.90 | 714.86 |
| 471 | Ac-F$r8AYWEAibL$NleA-NH₂ | 1196 | 1429.80 | 715.90 | 715.97 |
| 472 | Ac-F$r8AYWEAL$NleA-NH₂ | 1197 | 1415.79 | 708.90 | 708.94 |
| 473 | Ac-F$r8AYWENleL$NleA-NH₂ | 1198 | 1457.83 | 729.92 | 729.96 |
| 474 | Ac-F$r8AYWEAibL$Abu-NH₂ | 1199 | 1330.73 | 666.37 | 666.39 |
| 475 | Ac-F$r8AYWENleL$Abu-NH₂ | 1200 | 1358.76 | 680.38 | 680.39 |
| 476 | Ac-F$r8AYWEAL$Abu-NH₂ | 1201 | 1316.72 | 659.36 | 659.36 |
| 477 | Ac-LTF$r8AFWAQL$S-NH₂ | 1202 | 1515.85 | 758.93 | 759.12 |
| 478 | Ac-LTF$r8AWWAQL$S-NH₂ | 1203 | 1554.86 | 778.43 | 778.51 |
| 479 | Ac-LTF$r8AYWAQI$S-NH₂ | 1204 | 1531.84 | 766.92 | 766.96 |
| 480 | Ac-LTF$r8AYWAQNle$S-NH₂ | 1205 | 1531.84 | 766.92 | 766.96 |
| 481 | Ac-LTF$r8AYWAQL$SA-NH₂ | 1206 | 1602.88 | 802.44 | 802.48 |
| 482 | Ac-LTF$r8AWWAQL$A-NH₂ | 1207 | 1538.87 | 770.44 | 770.89 |
| 483 | Ac-LTFSr8AYWAQISA-NH₂ | 1208 | 1515.85 | 758.93 | 759.42 |
| 484 | Ac-LTF$r8AYWAQNle$A-NH₂ | 1209 | 1515.85 | 758.93 | 759.42 |
| 485 | Ac-LTFSr8AYWAQLSAA-NH₂ | 1210 | 1586.89 | 794.45 | 794.94 |
| 486 | Ac-LTF$r8HWWAQL$S-NH₂ | 1211 | 1620.88 | 811.44 | 811.47 |
| 487 | Ac-LTFSr8HRWAQLSS-NH₂ | 1212 | 1590.90 | 796.45 | 796.52 |
| 488 | Ac-LTF$r8HKWAQL$S-NH₂ | 1213 | 1562.90 | 782.45 | 782.53 |
| 489 | Ac-LTFSr8HYWAQLSW-NH₂ | 1214 | 1696.91 | 849.46 | 849.5 |
| 491 | Ac-F$r8AYWAbuAL$A-NH₂ | 1215 | 1258.71 | 630.36 | 630.5 |
| 492 | Ac-FSr8AbuYWEALSA-NH₂ | 1216 | 1316.72 | 659.36 | 659.51 |
| 493 | Ac-NlePRF%r8NYWRLL%QN-NH₂ | 1217 | 1954.13 | 978.07 | 978.54 |
| 494 | Ac-TSF%r8HYWAQL%S-NH₂ | 1218 | 1573.83 | 787.92 | 787.98 |
| 495 | Ac-LTF%r8AYWAQL%S-NH₂ | 1219 | 1533.86 | 767.93 | 768 |
| 496 | Ac-HTFSr8HYWAQLSS-NH₂ | 1220 | 1621.84 | 811.92 | 811.96 |
| 497 | Ac-LHFSr8HYWAQLSS-NH₂ | 1221 | 1633.88 | 817.94 | 818.02 |
| 498 | Ac-LTFSr8HHWAQLSS-NH₂ | 1222 | 1571.86 | 786.93 | 786.94 |
| 499 | Ac-LTFSr8HYWHQLSS-NH₂ | 1223 | 1663.89 | 832.95 | 832.38 |
| 500 | Ac-LTFSr8HYWAHLSS-NH₂ | 1224 | 1606.87 | 804.44 | 804.48 |
| 501 | Ac-LTFSr8HYWAQLSH-NH₂ | 1225 | 1647.89 | 824.95 | 824.98 |
| 502 | Ac-LTF$r8HYWAQL$S-NHPr | 1226 | 1639.91 | 820.96 | 820.98 |
| 503 | Ac-LTF$r8HYWAQL$S-NHsBu | 1227 | 1653.93 | 827.97 | 828.02 |
| 504 | Ac-LTF$r8HYWAQL$S-NHiBu | 1228 | 1653.93 | 827.97 | 828.02 |
| 505 | Ac-LTF$r8HYWAQL$S-NHBn | 1229 | 1687.91 | 844.96 | 844.44 |
| 506 | Ac-LTF$r8HYWAQL$S-NHPe | 1230 | 1700.92 | 851.46 | 851.99 |
| 507 | Ac-LTF$r8HYWAQL$S-NHChx | 1231 | 1679.94 | 840.97 | 841.04 |
| 508 | Ac-ETFSr8AYWAQLSS-NH₂ | 1232 | 1547.80 | 774.90 | 774.96 |
| 509 | Ac-STFSr8AYWAQLSS-NH₂ | 1233 | 1505.79 | 753.90 | 753.94 |
| 510 | Ac-LEFSr8AYWAQLSS-NH₂ | 1234 | 1559.84 | 780.92 | 781.25 |
| 511 | Ac-LSFSr8AYWAQLSS-NH₂ | 1235 | 1517.83 | 759.92 | 759.93 |
| 512 | Ac-LTFSr8EYWAQLSS-NH₂ | 1236 | 1589.85 | 795.93 | 795.97 |
| 513 | Ac-LTFSr8SYWAQLSS-NH₂ | 1237 | 1547.84 | 774.92 | 774.96 |
| 514 | Ac-LTFSr8AYWEQLSS-NH₂ | 1238 | 1589.85 | 795.93 | 795.9 |
| 515 | Ac-LTFSr8AYWAELSS-NH₂ | 1239 | 1532.83 | 767.42 | 766.96 |
| 516 | Ac-LTFSr8AYWASLSS-NH₂ | 1240 | 1490.82 | 746.41 | 746.46 |
| 517 | Ac-LTFSr8AYWAQLSE-NH₂ | 1241 | 1573.85 | 787.93 | 787.98 |
| 518 | Ac-LTF2CNSr8HYWAQLSS-NH₂ | 1242 | 1622.86 | 812.43 | 812.47 |
| 519 | Ac-LTF3ClSr8HYWAQLSS-NH₂ | 1243 | 1631.83 | 816.92 | 816.99 |
| 520 | Ac-LTDipSr8HYWAQLSS-NH₂ | 1244 | 1673.90 | 837.95 | 838.01 |
| 521 | Ac-LTFSr8HYWAQTle$S-NH₂ | 1245 | 1597.87 | 799.94 | 800.04 |
| 522 | Ac-F$r8AY6clWEAL$A-NH₂ | 1246 | 1336.66 | 669.33 | 1338.56 |
| 523 | Ac-F$r8AYdl6brWEAL$A-NH₂ | 1247 | 1380.61 | 691.31 | 692.2 |
| 524 | Ac-F$r8AYdl6fWEAL$A-NH₂ | 1248 | 1320.69 | 661.35 | 1321.61 |
| 525 | Ac-F$r8AYdl4mWEAL$A-NH₂ | 1249 | 1316.72 | 659.36 | 659.36 |
| 526 | Ac-F$r8AYdl5clWEAL$A-NH₂ | 1250 | 1336.66 | 669.33 | 669.35 |
| 527 | Ac-F$r8AYdl7mWEAL$A-NH₂ | 1251 | 1316.72 | 659.36 | 659.36 |
| 528 | Ac-LTF%r8HYWAQL%A-NH₂ | 1252 | 1583.89 | 792.95 | 793.01 |
| 529 | Ac-LTF$r8HCouWAQL$S-NH₂ | 1253 | 1679.87 | 840.94 | 841.38 |
| 530 | Ac-LTFEHCouWAQLTS-NH₂ | 1254 | 1617.75 | 809.88 | 809.96 |
| 531 | Ac-LTA$r8HCouWAQL$S-NH₂ | 1255 | 1603.84 | 802.92 | 803.36 |
| 532 | Ac-F$r8AYWEAL$AbuA-NH₂ | 1256 | 1387.75 | 694.88 | 694.88 |
| 533 | Ac-F$r8AYWEAI$AA-NH₂ | 1257 | 1373.74 | 687.87 | 687.93 |
| 534 | Ac-F$r8AYWEANle$AA-NH₂ | 1258 | 1373.74 | 687.87 | 687.93 |
| 535 | Ac-F$r8AYWEAmlL$AA-NH₂ | 1259 | 1429.80 | 715.90 | 715.97 |
| 536 | Ac-F$r8AYWQAL$AA-NH₂ | 1260 | 1372.75 | 687.38 | 687.48 |
| 537 | Ac-F$r8AYWAAL$AA-NH₂ | 1261 | 1315.73 | 658.87 | 658.92 |
| 538 | Ac-F$r8AYWAbuAL$AA-NH₂ | 1262 | 1329.75 | 665.88 | 665.95 |
| 539 | Ac-F$r8AYWNleAL$AA-NH₂ | 1263 | 1357.78 | 679.89 | 679.94 |
| 540 | Ac-F$r8AbuYWEAL$AA-NH₂ | 1264 | 1387.75 | 694.88 | 694.96 |
| 541 | Ac-F$r8NleYWEAL$AA-NH₂ | 1265 | 1415.79 | 708.90 | 708.94 |
| 542 | Ac-F$r8FYWEAL$AA-NH₂ | 1266 | 1449.77 | 725.89 | 725.97 |
| 543 | Ac-LTF$r8HYWAQhL$S-NH₂ | 1267 | 1611.88 | 806.94 | 807 |
| 544 | Ac-LTF$r8HYWAQAdm$S-NH₂ | 1268 | 1675.91 | 838.96 | 839.04 |
| 545 | Ac-LTF$r8HYWAQIgl$S-NH₂ | 1269 | 1659.88 | 830.94 | 829.94 |
| 546 | Ac-F$r8AYWAQL$AA-NH₂ | 1270 | 1372.75 | 687.38 | 687.48 |
| 547 | Ac-LTF$r8ALWAQL$Q-NH₂ | 1271 | 1522.89 | 762.45 | 762.52 |
| 548 | Ac-F$r8AYWEAL$AA-NH₂ | 1272 | 1373.74 | 687.87 | 687.93 |
| 549 | Ac-F$r8AYWENleL$AA-NH₂ | 1273 | 1415.79 | 708.90 | 708.94 |
| 550 | Ac-F$r8AYWEAibL$Abu-NH₂ | 1274 | 1330.73 | 666.37 | 666.39 |
| 551 | Ac-F$r8AYWENleL$Abu-NH₂ | 1275 | 1358.76 | 680.38 | 680.38 |
| 552 | Ac-F$r8AYWEAL$Abu-NH₂ | 1276 | 1316.72 | 659.36 | 659.36 |
| 553 | Ac-F$r8AYWEAc3cL$AbuA-NH₂ | 1277 | 1399.75 | 700.88 | 700.95 |
| 554 | Ac-F$r8AYWEAc3cL$NleA-NH₂ | 1278 | 1427.79 | 714.90 | 715.01 |
| 555 | H-LTF$r8AYWAQL$S-NH₂ | 1279 | 1489.83 | 745.92 | 745.95 |
| 556 | mdPEG3-LTF$r8AYWAQL$S-NH₂ | 1280 | 1679.92 | 840.96 | 840.97 |
| 557 | mdPEG7-LTF$r8AYWAQL$S-NH₂ | 1281 | 1856.02 | 929.01 | 929.03 |
| 558 | Ac-F$r8ApmpEt6clWEAL$A-NH₂ | 1282 | 1470.71 | 736.36 | 788.17 |
| 559 | Ac-LTF3Cl$r8AYWAQL$S-NH₂ | 1283 | 1565.81 | 783.91 | 809.18 |
| 560 | Ac-LTF3Cl$r8HYWAQL$A-NH₂ | 1284 | 1615.83 | 808.92 | 875.24 |
| 561 | Ac-LTF3Cl$r8HYWWQL$S-NH₂ | 1285 | 1746.87 | 874.44 | 841.65 |
| 562 | Ac-LTF3Cl$r8AYWWQL$S-NH₂ | 1286 | 1680.85 | 841.43 | 824.63 |
| 563 | Ac-LTF$r8AYWWQL$S-NH₂ | 1287 | 1646.89 | 824.45 | 849.98 |
| 564 | Ac-LTF$r8HYWWQL$A-NH₂ | 1288 | 1696.91 | 849.46 | 816.67 |
| 565 | Ac-LTF$r8AYWWQL$A-NH₂ | 1289 | 1630.89 | 816.45 | 776.15 |
| 566 | Ac-LTF4F$r8AYWAQL$S-NH₂ | 1290 | 1549.83 | 775.92 | 776.15 |
| 567 | Ac-LTF2F$r8AYWAQL$S-NH₂ | 1291 | 1549.83 | 775.92 | 776.15 |
| 568 | Ac-LTF3F$r8AYWAQL$S-NH₂ | 1292 | 1549.83 | 775.92 | 785.12 |
| 569 | Ac-LTF34F2$r8AYWAQL$S-NH₂ | 1293 | 1567.83 | 784.92 | 785.12 |
| 570 | Ac-LTF35F2$r8AYWAQL$S-NH₂ | 1294 | 1567.83 | 784.92 | 1338.74 |
| 571 | Ac-F3Cl$r8AYWEAL$A-NH₂ | 1295 | 1336.66 | 669.33 | 705.28 |
| 572 | Ac-F3Cl$r8AYWEAL$AA-NH₂ | 1296 | 1407.70 | 704.85 | 680.11 |
| 573 | Ac-F$r8AY6clWEAL$AA-NH₂ | 1297 | 1407.70 | 704.85 | 736.83 |
| 574 | Ac-F$r8AY6clWEAL$-NH₂ | 1298 | 1265.63 | 633.82 | 784.1 |
| 575 | Ac-LTF$r8HYWAQLSt/S-NH₂ | 1299 | 16.03 | 9.02 | 826.98 |
| 576 | Ac-LTF$r8HYWAQL$S-NHsBu | 1300 | 1653.93 | 827.97 | 828.02 |
| 577 | Ac-STF$r8AYWAQL$S-NH₂ | 1301 | 1505.79 | 753.90 | 753.94 |
| 578 | Ac-LTF$r8AYWAEL$S-NH₂ | 1302 | 1532.83 | 767.42 | 767.41 |
| 579 | Ac-LTF$r8AYWAQL$E-NH₂ | 1303 | 1573.85 | 787.93 | 787.98 |
| 580 | mdPEG3-LTF$r8AYWAQL$S-NH₂ | 1304 | 1679.92 | 840.96 | 840.97 |
| 581 | Ac-LTF$r8AYWAQhL$S-NH₂ | 1305 | 1545.86 | 773.93 | 774.31 |
| 583 | Ac-LTF$r8AYWAQCha$S-NH₂ | 1306 | 1571.88 | 786.94 | 787.3 |
| 584 | Ac-LTF$r8AYWAQChg$S-NH₂ | 1307 | 1557.86 | 779.93 | 780.4 |
| 585 | Ac-LTF$r8AYWAQCba$S-NH₂ | 1308 | 1543.84 | 772.92 | 780.13 |
| 586 | Ac-LTF$r8AYWAQF$S-NH₂ | 1309 | 1565.83 | 783.92 | 784.2 |
| 587 | Ac-LTF4F$r8HYWAQhL$S-NH₂ | 1310 | 1629.87 | 815.94 | 815.36 |
| 588 | Ac-LTF4F$r8HYWAQCha$S-NH₂ | 1311 | 1655.89 | 828.95 | 828.39 |
| 589 | Ac-LTF4F$r8HYWAQChg$S-NH₂ | 1312 | 1641.87 | 821.94 | 821.35 |
| 590 | Ac-LTF4F$r8HYWAQCba$S-NH₂ | 1313 | 1627.86 | 814.93 | 814.32 |
| 591 | Ac-LTF4F$r8AYWAQhL$S-NH₂ | 1314 | 1563.85 | 782.93 | 782.36 |
| 592 | Ac-LTF4F$r8AYWAQCha$S-NH₂ | 1315 | 1589.87 | 795.94 | 795.38 |
| 593 | Ac-LTF4F$r8AYWAQChg$S-NH₂ | 1316 | 1575.85 | 788.93 | 788.35 |
| 594 | Ac-LTF4F$r8AYWAQCba$S-NH₂ | 1317 | 1561.83 | 781.92 | 781.39 |
| 595 | Ac-LTF3Cl$r8AYWAQhL$S-NH₂ | 1318 | 1579.82 | 790.91 | 790.35 |
| 596 | Ac-LTF3Cl$r8AYWAQCha$S-NH₂ | 1319 | 1605.84 | 803.92 | 803.67 |
| 597 | Ac-LTF3Cl$r8AYWAQChg$S-NH₂ | 1320 | 1591.82 | 796.91 | 796.34 |
| 598 | Ac-LTF3Cl$r8AYWAQCba$S-NH₂ | 1321 | 1577.81 | 789.91 | 789.39 |
| 599 | Ac-LTF$r8AYWAQhF$S-NH₂ | 1322 | 1579.84 | 790.92 | 791.14 |
| 600 | Ac-LTF$r8AYWAQF3CF3$S-NH₂ | 1323 | 1633.82 | 817.91 | 818.15 |
| 601 | Ac-LTF$r8AYWAQF3Me$S-NH₂ | 1324 | 1581.86 | 791.93 | 791.32 |
| 602 | Ac-LTF$r8AYWAQ1Nal$S-NH₂ | 1325 | 1615.84 | 808.92 | 809.18 |
| 603 | Ac-LTF$r8AYWAQBip$S-NH₂ | 1326 | 1641.86 | 821.93 | 822.13 |
| 604 | Ac-LTF$r8FYWAQL$A-NH₂ | 1327 | 1591.88 | 796.94 | 797.33 |
| 605 | Ac-LTF$r8HYWAQL$S-NHAm | 1328 | 1667.94 | 834.97 | 835.92 |
| 606 | Ac-LTF$r8HYWAQL$S-NHiAm | 1329 | 1667.94 | 834.97 | 835.55 |
| 607 | Ac-LTF$r8HYWAQL$S-NHnPr3Ph | 1330 | 1715.94 | 858.97 | 859.79 |
| 608 | Ac-LTF$r8HYWAQL$S-NHnBu3, 3Me | 1331 | 1681.96 | 841.98 | 842.49 |
| 610 | Ac-LTF$r8HYWAQL$S-NHnPr | 1332 | 1639.91 | 820.96 | 821.58 |
| 611 | Ac-LTF$r8HYWAQL$S-NHnEt2Ch | 1333 | 1707.98 | 854.99 | 855.35 |
| 612 | Ac-LTF$r8HYWAQL$S-NHHex | 1334 | 1681.96 | 841.98 | 842.4 |
| 613 | Ac-LTF$r8AYWAQL$S-NHmdPeg2 | 1335 | 1633.91 | 817.96 | 818.35 |
| 614 | Ac-LTF$r8AYWAQL$A-NHmdPeg2 | 1336 | 1617.92 | 809.96 | 810.3 |
| 615 | Ac-LTF$r8AYwAQL$A-NHmdPeg4 | 1337 | 1705.97 | 853.99 | 854.33 |
| 616 | Ac-F$r8AYd14mwEAL$A-NH₂ | 1338 | 1316.72 | 659.36 | 659.44 |
| 617 | Ac-F$r8AYdl5clWEAL$A-NH₂ | 1339 | 1336.66 | 669.33 | 669.43 |
| 618 | Ac-LThF$r8AYWAQL$S-NH₂ | 1340 | 1545.86 | 773.93 | 774.11 |
| 619 | Ac-LT2Nal$r8AYwAQL$S-NH₂ | 1341 | 1581.86 | 791.93 | 792.43 |
| 620 | Ac-LTA$r8AYWAQL$S-NH₂ | 1342 | 1455.81 | 728.91 | 729.15 |
| 621 | Ac-LTF$r8AYWVQL$S-NH₂ | 1343 | 1559.88 | 780.94 | 781.24 |
| 622 | Ac-LTF$r8HYWAAL$A-NH₂ | 1344 | 1524.85 | 763.43 | 763.86 |
| 623 | Ac-LTF$r8VYWAQL$A-NH₂ | 1345 | 1543.88 | 772.94 | 773.37 |
| 624 | Ac-LTF$r8IYWAQL$S-NH₂ | 1346 | 1573.89 | 787.95 | 788.17 |
| 625 | Ac-FTF$r8VYWSQL$S-NH₂ | 1347 | 1609.85 | 805.93 | 806.22 |
| 626 | Ac-ITF$r8FYWAQL$S-NH₂ | 1348 | 1607.88 | 804.94 | 805.2 |
| 627 | Ac-2NalTF$r8VYWSQL$S-NH₂ | 1349 | 1659.87 | 830.94 | 831.2 |
| 628 | Ac-ITF$r8LYWSQL$S-NH₂ | 1350 | 1589.89 | 795.95 | 796.13 |
| 629 | Ac-FTF$r8FYWAQL$S-NH₂ | 1351 | 1641.86 | 821.93 | 822.13 |
| 630 | Ac-WTF$r8VYWAQL$S-NH₂ | 1352 | 1632.87 | 817.44 | 817.69 |
| 631 | Ac-WTF$r8WYWAQL$S-NH₂ | 1353 | 1719.88 | 860.94 | 861.36 |
| 632 | Ac-VTF$r8AYWSQL$S-NH₂ | 1354 | 1533.82 | 767.91 | 768.19 |
| 633 | Ac-WTF$r8FYWSQL$S-NH₂ | 1355 | 1696.87 | 849.44 | 849.7 |
| 634 | Ac-FTF$r8IYWAQL$S-NH₂ | 1356 | 1607.88 | 804.94 | 805.2 |
| 635 | Ac-WTF$r8VYWSQL$S-NH₂ | 1357 | 1648.87 | 825.44 | 824.8 |
| 636 | Ac-FTF$r8LYWSQL$S-NH₂ | 1358 | 1623.87 | 812.94 | 812.8 |
| 637 | Ac-YTF$r8FYWSQL$S-NH₂ | 1359 | 1673.85 | 837.93 | 837.8 |
| 638 | Ac-LTF$r8AY6clWEAL$A-NH₂ | 1360 | 1550.79 | 776.40 | 776.14 |
| 639 | Ac-LTF$r8AY6clWSQL$S-NH₂ | 1361 | 1581.80 | 791.90 | 791.68 |
| 640 | Ac-F$r8AY6clWSAL$A-NH₂ | 1362 | 1294.65 | 648.33 | 647.67 |
| 641 | Ac-F$r8AY6clWQAL$AA-NH₂ | 1363 | 1406.72 | 704.36 | 703.84 |
| 642 | Ac-LHF$r8AYWAQL$S-NH₂ | 1364 | 1567.86 | 784.93 | 785.21 |
| 643 | Ac-LTF$r8AYWAQL$S-NH₂ | 1365 | 1531.84 | 766.92 | 767.17 |
| 644 | Ac-LTF$r8AHWAQL$S-NH₂ | 1366 | 1505.84 | 753.92 | 754.13 |
| 645 | Ac-LTF$r8AYWAHL$S-NH₂ | 1367 | 1540.84 | 771.42 | 771.61 |
| 646 | Ac-LTF$r8AYWAQL$H-NH₂ | 1368 | 1581.87 | 791.94 | 792.15 |
| 647 | H-LTF$r8AYWAQL$A-NH₂ | 1369 | 1473.84 | 737.92 | 737.29 |
| 648 | Ac-HHF$r8AYWAQL$S-NH₂ | 1370 | 1591.83 | 796.92 | 797.35 |
| 649 | Ac-aAibWTF$r8VYWSQL$S-NH₂ | 1371 | 1804.96 | 903.48 | 903.64 |
| 650 | Ac-AibWTF$r8HYWAQL$S-NH₂ | 1372 | 1755.91 | 878.96 | 879.4 |
| 651 | Ac-AibAWTF$r8HYWAQL$S-NH₂ | 1373 | 1826.95 | 914.48 | 914.7 |
| 652 | Ac-fWTF$r8HYWAQL$S-NH₂ | 1374 | 1817.93 | 909.97 | 910.1 |
| 653 | Ac-AibWWTF$r8HYWAQL$S-NH₂ | 1375 | 1941.99 | 972.00 | 972.2 |
| 654 | Ac-WTF$r8LYWSQL$S-NH₂ | 1376 | 1662.88 | 832.44 | 832.8 |
| 655 | Ac-WTF$r8NleYWSQL$S-NH₂ | 1377 | 1662.88 | 832.44 | 832.6 |
| 656 | Ac-LTF$r8AYWSQL$a-NH₂ | 1378 | 1531.84 | 766.92 | 767.2 |
| 657 | Ac-LTF$r8EYWARL$A-NH₂ | 1379 | 1601.90 | 801.95 | 802.1 |
| 658 | Ac-LTF$r8EYWAHL$A-NH₂ | 1380 | 1582.86 | 792.43 | 792.6 |
| 659 | Ac-aTF$r8AYWAQL$S-NH₂ | 1381 | 1489.80 | 745.90 | 746.08 |
| 660 | Ac-AibTF$r8AYWAQL$S-NH₂ | 1382 | 1503.81 | 752.91 | 753.11 |
| 661 | Ac-AmfTF$r8AYWAQL$S-NH₂ | 1383 | 1579.84 | 790.92 | 791.14 |
| 662 | Ac-AmwTF$r8AYWAQL$S-NH₂ | 1384 | 1618.86 | 810.43 | 810.66 |
| 663 | Ac-NmLTF$r8AYWAQL$S-NH₂ | 1385 | 1545.86 | 773.93 | 774.11 |
| 664 | Ac-LNmTF$r8AYWAQL$S-NH₂ | 1386 | 1545.86 | 773.93 | 774.11 |
| 665 | Ac-LSarF$r8AYWAQL$S-NH₂ | 1387 | 1501.83 | 751.92 | 752.18 |
| 667 | Ac-LGF$r8AYWAQL$S-NH₂ | 1388 | 1487.82 | 744.91 | 745.15 |
| 668 | Ac-LTNmF$r8AYWAQL$S-NH₂ | 1389 | 1545.86 | 773.93 | 774.2 |
| 669 | Ac-TF$r8AYWAQL$S-NH₂ | 1390 | 1418.76 | 710.38 | 710.64 |
| 670 | Ac-ETF$r8AYWAQL$A-NH₂ | 1391 | 1531.81 | 766.91 | 767.2 |
| 671 | Ac-LTF$r8EYWAQL$A-NH₂ | 1392 | 1573.85 | 787.93 | 788.1 |
| 672 | Ac-LT2Nal$r8AYWSQL$S-NH₂ | 1393 | 1597.85 | 799.93 | 800.4 |
| 673 | Ac-LTF$r8AYWAAL$S-NH₂ | 1394 | 1474.82 | 738.41 | 738.68 |
| 674 | Ac-LTF$r8AYWAQhCha$S-NH₂ | 1395 | 1585.89 | 793.95 | 794.19 |
| 675 | Ac-LTF$r8AYWAQChg$S-NH₂ | 1396 | 1557.86 | 779.93 | 780.97 |
| 676 | Ac-LTF$r8AYWAQCba$S-NH₂ | 1397 | 1543.84 | 772.92 | 773.19 |
| 677 | Ac-LTF$r8AYWAQF3CF3$S-NH₂ | 1398 | 1633.82 | 817.91 | 818.15 |
| 678 | Ac-LTF$r8AYWAQ1Nal$S-NH₂ | 1399 | 1615.84 | 808.92 | 809.18 |
| 679 | Ac-LTF$r8AYWAQBip$S-NH₂ | 1400 | 1641.86 | 821.93 | 822.32 |
| 680 | Ac-LT2Nal$r8AYWAQL$S-NH₂ | 1401 | 1581.86 | 791.93 | 792.15 |
| 681 | Ac-LTF$r8AYWVQL$S-NH₂ | 1402 | 1559.88 | 780.94 | 781.62 |
| 682 | Ac-LTF$r8AWWAQL$S-NH₂ | 1403 | 1554.86 | 778.43 | 778.65 |
| 683 | Ac-FTF$r8VYWSQL$S-NH₂ | 1404 | 1609.85 | 805.93 | 806.12 |
| 684 | Ac-ITF$r8FYWAQL$S-NH₂ | 1405 | 1607.88 | 804.94 | 805.2 |
| 685 | Ac-ITF$r8LYWSQL$S-NH₂ | 1406 | 1589.89 | 795.95 | 796.22 |
| 686 | Ac-FTF$r8FYWAQL$S-NH₂ | 1407 | 1641.86 | 821.93 | 822.41 |
| 687 | Ac-VTF$r8AYWSQL$S-NH₂ | 1408 | 1533.82 | 767.91 | 768.19 |
| 688 | Ac-LTF$r8AHWAQL$S-NH₂ | 1409 | 1505.84 | 753.92 | 754.31 |
| 689 | Ac-LTF$r8AYWAQL$H-NH₂ | 1410 | 1581.87 | 791.94 | 791.94 |
| 690 | Ac-LTF$r8AYWAHL$S-NH₂ | 1411 | 1540.84 | 771.42 | 771.61 |
| 691 | Ac-aAibWTF$r8VYWSQL$S-NH₂ | 1412 | 1804.96 | 903.48 | 903.9 |
| 692 | Ac-AibWTF$r8HYWAQL$S-NH₂ | 1413 | 1755.91 | 878.96 | 879.5 |
| 693 | Ac-AibAWTF$r8HYWAQL$S-NH₂ | 1414 | 1826.95 | 914.48 | 914.7 |
| 694 | Ac-fWTF$r8HYWAQL$S-NH₂ | 1415 | 1817.93 | 909.97 | 910.2 |
| 695 | Ac-AibWWTF$r8HYWAQL$S-NH₂ | 1416 | 1941.99 | 972.00 | 972.7 |
| 696 | Ac-WTF$r8LYWSQL$S-NH₂ | 1417 | 1662.88 | 832.44 | 832.7 |
| 697 | Ac-WTF$r8NleYWSQL$S-NH₂ | 1418 | 1662.88 | 832.44 | 832.7 |
| 698 | Ac-LTF$r8AYWSQL$a-NH₂ | 1419 | 1531.84 | 766.92 | 767.2 |
| 699 | Ac-LTF$r8EYWARL$A-NH₂ | 1420 | 1601.90 | 801.95 | 802.2 |
| 700 | Ac-LTF$r8EYWAHL$A-NH₂ | 1421 | 1582.86 | 792.43 | 792.6 |
| 701 | Ac-aTF$r8AYWAQL$S-NH₂ | 1422 | 1489.80 | 745.90 | 746.1 |
| 702 | Ac-AibTF$r8AYWAQL$S-NH₂ | 1423 | 1503.81 | 752.91 | 753.2 |
| 703 | Ac-AmfTF$r8AYWAQL$S-NH₂ | 1424 | 1579.84 | 790.92 | 791.2 |
| 704 | Ac-AmwTF$r8AYWAQL$S-NH₂ | 1425 | 1618.86 | 810.43 | 810.7 |
| 705 | Ac-NmLTF$r8AYWAQL$S-NH₂ | 1426 | 1545.86 | 773.93 | 774.1 |
| 706 | Ac-LNmTF$r8AYWAQL$S-NH₂ | 1427 | 1545.86 | 773.93 | 774.4 |
| 707 | Ac-LSarF$r8AYWAQL$S-NH₂ | 1428 | 1501.83 | 751.92 | 752.1 |
| 708 | Ac-TF$r8AYWAQL$S-NH₂ | 1429 | 1418.76 | 710.38 | 710.8 |
| 709 | Ac-ETF$r8AYWAQL$A-NH₂ | 1430 | 1531.81 | 766.91 | 767.4 |
| 710 | Ac-LTF$r8EYWAQL$A-NH₂ | 1431 | 1573.85 | 787.93 | 788.2 |
| 711 | Ac-WTF$r8VYWSQL$S-NH₂ | 1432 | 1648.87 | 825.44 | 825.2 |
| 713 | Ac-YTF$r8FYWSQL$S-NH₂ | 1433 | 1673.85 | 837.93 | 837.3 |
| 714 | Ac-F$r8AY6clWSAL$A-NH₂ | 1434 | 1294.65 | 648.33 | 647.74 |
| 715 | Ac-ETF$r8EYWVQL$S-NH₂ | 1435 | 1633.84 | 817.92 | 817.36 |
| 716 | Ac-ETF$r8EHWAQL$A-NH₂ | 1436 | 1563.81 | 782.91 | 782.36 |
| 717 | Ac-ITF$r8EYWAQL$S-NH₂ | 1437 | 1589.85 | 795.93 | 795.38 |
| 718 | Ac-ITF$r8EHWVQL$A-NH₂ | 1438 | 1575.88 | 788.94 | 788.42 |
| 719 | Ac-ITF$r8EHWAQL$S-NH₂ | 1439 | 1563.85 | 782.93 | 782.43 |
| 720 | Ac-LTF4F$r8AYWAQCba$S-NH₂ | 1440 | 1561.83 | 781.92 | 781.32 |
| 721 | Ac-LTF3Cl$r8AYWAQhL$S-NH₂ | 1441 | 1579.82 | 790.91 | 790.64 |
| 722 | Ac-LTF3Cl$r8AYWAQCha$S-NH₂ | 1442 | 1605.84 | 803.92 | 803.37 |
| 723 | Ac-LTF3Cl$r8AYWAQChg$S-NH₂ | 1443 | 1591.82 | 796.91 | 796.27 |
| 724 | Ac-LTF3Cl$r8AYWAQCba$S-NH₂ | 1444 | 1577.81 | 789.91 | 789.83 |
| 725 | Ac-LTF$r8AY6clWSQL$S-NH₂ | 1445 | 1581.80 | 791.90 | 791.75 |
| 726 | Ac-LTF4F$r8HYWAQhL$S-NH₂ | 1446 | 1629.87 | 815.94 | 815.36 |
| 727 | Ac-LTF4F$r8HYWAQCba$S-NH₂ | 1447 | 1627.86 | 814.93 | 814.32 |
| 728 | Ac-LTF4F$r8AYWAQhL$S-NH₂ | 1448 | 1563.85 | 782.93 | 782.36 |
| 729 | Ac-LTF4F$r8AYWAQChg$S-NH₂ | 1449 | 1575.85 | 788.93 | 788.35 |
| 730 | Ac-ETF$r8EYWVAL$S-NH₂ | 1450 | 1576.82 | 789.41 | 788.79 |
| 731 | Ac-ETF$r8EHWAAL$A-NH₂ | 1451 | 1506.79 | 754.40 | 754.8 |
| 732 | Ac-ITF$r8EYWAAL$S-NH₂ | 1452 | 1532.83 | 767.42 | 767.75 |
| 733 | Ac-ITF$r8EHWVAL$A-NH₂ | 1453 | 1518.86 | 760.43 | 760.81 |
| 734 | Ac-ITF$r8EHWAAL$S-NH₂ | 1454 | 1506.82 | 754.41 | 754.8 |
| 735 | Pam-LTF$r8EYWAQL$S-NH₂ | 1455 | 1786.07 | 894.04 | 894.48 |
| 736 | Pam-ETF$r8EYWAQL$S-NH₂ | 1456 | 1802.03 | 902.02 | 902.34 |
| 737 | Ac-LTF$r8AYWLQL$S-NH₂ | 1457 | 1573.89 | 787.95 | 787.39 |
| 738 | Ac-LTF$r8EYWLQL$S-NH₂ | 1458 | 1631.90 | 816.95 | 817.33 |
| 739 | Ac-LTF$r8EHWLQL$S-NH₂ | 1459 | 1605.89 | 803.95 | 804.29 |
| 740 | Ac-LTF$r8VYWAQL$S-NH₂ | 1460 | 1559.88 | 780.94 | 781.34 |
| 741 | Ac-LTF$r8AYWSQL$S-NH₂ | 1461 | 1547.84 | 774.92 | 775.33 |
| 742 | Ac-ETF$r8AYWAQL$S-NH₂ | 1462 | 1547.80 | 774.90 | 775.7 |
| 743 | Ac-LTF$r8EYWAQL$S-NH₂ | 1463 | 1589.85 | 795.93 | 796.33 |
| 744 | Ac-LTF$r8HYWAQL$S-NHAm | 1464 | 1667.94 | 834.97 | 835.37 |
| 745 | Ac-LTF$r8HYWAQL$S-NHiAm | 1465 | 1667.94 | 834.97 | 835.27 |
| 746 | Ac-LTF$r8HYWAQL$S-NHnPr3Ph | 1466 | 1715.94 | 858.97 | 859.42 |
| 747 | Ac-LTF$r8HYWAQL$S-NHnBu3, 3Me | 1467 | 1681.96 | 841.98 | 842.67 |
| 748 | Ac-LTF$r8HYWAQL$S-NHnBu | 1468 | 1653.93 | 827.97 | 828.24 |
| 749 | Ac-LTF$r8HYWAQL$S-NHnPr | 1469 | 1639.91 | 820.96 | 821.31 |
| 750 | Ac-LTF$r8HYWAQL$S-NHnEt2Ch | 1470 | 1707.98 | 854.99 | 855.35 |
| 751 | Ac-LTF$r8HYWAQL$S-NHHex | 1471 | 1681.96 | 841.98 | 842.4 |
| 752 | Ac-LTF$r8AYWAQL$S-NHmdPeg2 | 1472 | 1633.91 | 817.96 | 855.35 |
| 753 | Ac-LTF$r8AYWAQL$A-NHmdPeg2 | 1473 | 1617.92 | 809.96 | 810.58 |
| 754 | Ac-LTF$r5AYWAAL$s8S-NH₂ | 1474 | 1474.82 | 738.41 | 738.79 |
| 755 | Ac-LTF$r8AYWCouQL$S-NH₂ | 1475 | 1705.88 | 853.94 | 854.61 |
| 756 | Ac-LTF$r8CouYWAQL$S-NH₂ | 1476 | 1705.88 | 853.94 | 854.7 |
| 757 | Ac-CouTF$r8AYWAQL$S-NH₂ | 1477 | 1663.83 | 832.92 | 833.33 |
| 758 | H-LTF$r8AYWAQL$A-NH₂ | 1478 | 1473.84 | 737.92 | 737.29 |
| 759 | Ac-HHF$r8AYWAQL$S-NH₂ | 1479 | 1591.83 | 796.92 | 797.72 |
| 760 | Ac-LT2Nal$r8AYWSQL$S-NH₂ | 1480 | 1597.85 | 799.93 | 800.68 |
| 761 | Ac-LTF$r8HCouWAQL$S-NH₂ | 1481 | 1679.87 | 840.94 | 841.38 |
| 762 | Ac-LTF$r8AYWCou2QL$S-NH₂ | 1482 | 1789.94 | 895.97 | 896.51 |
| 763 | Ac-LTF$r8Cou2YWAQL$S-NH₂ | 1483 | 1789.94 | 895.97 | 896.5 |
| 764 | Ac-Cou2TF$r8AYWAQL$S-NH₂ | 1484 | 1747.90 | 874.95 | 875.42 |
| 765 | Ac-LTF$r8ACou2WAQL$S-NH₂ | 1485 | 1697.92 | 849.96 | 850.82 |
| 766 | Dmaac-LTF$r8AYWAQL$S-NH₂ | 1486 | 1574.89 | 788.45 | 788.82 |
| 767 | Hexac-LTF$r8AYWAQL$S-NH₂ | 1487 | 1587.91 | 794.96 | 795.11 |
| 768 | Napac-LTF$r8AYWAQL$S-NH₂ | 1488 | 1657.89 | 829.95 | 830.36 |
| 769 | Pam-LTF$r8AYWAQL$S-NH₂ | 1489 | 1728.06 | 865.03 | 865.45 |
| 770 | Ac-LT2Nal$r8HYAAQL$S-NH₂ | 1490 | 1532.84 | 767.42 | 767.61 |
| 771 | Ac-LT2Nal$/r8HYWAQL$/S-NH₂ | 1491 | 1675.91 | 838.96 | 839.1 |
| 772 | Ac-LT2Nal$r8HYFAQL$S-NH₂ | 1492 | 1608.87 | 805.44 | 805.9 |
| 773 | Ac-LT2Nal$r8HWAAQL$S-NH₂ | 1493 | 1555.86 | 778.93 | 779.08 |
| 774 | Ac-LT2Nal$r8HYAWQL$S-NH₂ | 1494 | 1647.88 | 824.94 | 825.04 |
| 775 | Ac-LT2Nal$r8HYAAQW$S-NH₂ | 1495 | 1605.83 | 803.92 | 804.05 |
| 776 | Ac-LTW$r8HYWAQL$S-NH₂ | 1496 | 1636.88 | 819.44 | 819.95 |
| 777 | Ac-LT1Nal$r8HYWAQL$S-NH₂ | 1497 | 1647.88 | 824.94 | 825.41 |

In some embodiments, a peptidomimetic macrocycles disclosed herein does not comprise a peptidomimetic macrocycle structure as shown in TABLE 2b.

TABLE 2c shows examples of non-crosslinked polypeptides comprising D-amino acids.

TABLE 2C

| | | SEQ | | Exact | Found | Calc | Calc | Calc |
|---|---|---|---|---|---|---|---|---|
| SP | Sequence | ID NO: | Isomer | Mass | Mass | (M + 1)/1 | (M + 2)/2 | (M + 3)/3 |
| 765 | Ac-tawyanfekllr-NH₂ | 1498 | 777.46 | | | | | |
| 766 | Ac-tawyanf4CF3ekllr-NH₂ | 1499 | 811.41 | | | | | |

### Example 3: Preparation of Peptidomimetic Macrocycles Using a Boc-Protected Amino Acid

Peptidomimetic macrocycle precursors comprising an R8 amino acid at position “i” and an S5 amino acid at position “i+7” were prepared. The amino acid at position “i+3” was a Boc-protected tryptophan, which was incorporated during solid-phase synthesis. Specifically, the Boc-protected tryptophan amino acid shown below was used during solid phase synthesis:

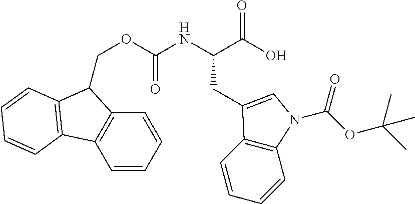 [SMILES: CC(C)(C)OC(=O)n1cc(C[C@H](NC(=O)OCC2c3ccccc3-c3ccccc32)C(=O)O)c2ccccc21]

Metathesis was performed using a ruthenium catalyst prior to the cleavage and deprotection steps. The composition obtained following cyclization was determined by HPLC analysis, and was found to contain primarily peptidomimetic macrocycles having a crosslinker comprising a trans olefin (“iso2”, comprising the double bond in an E configuration). Unexpectedly, a ratio of 90:10 was observed for the trans and cis products, respectively.

### Example 4: Preparation of Peptidomimetic Macrocycles Using a Boc-Protected Amino Acid

Peptidomimetic macrocycles were first dissolved in neat N, N-dimethylacetamide (DMA) to make 20× stock solutions over a concentration range of 20-140 mg/mL. The DMA stock solutions were diluted 20-fold in an aqueous vehicle containing 2% Solutol-HS-15, 25 mM histidine, and 45 mg/mL mannitol to obtain final concentrations of 1-7 mg/ml of the peptidomimetic macrocycles in 5% DMA, 2% Solutol-HS-15, 25 mM histidine, and 45 mg/mL mannitol. The final solutions were mixed gently by repeat pipetting or light vortexing. The final solutions were sonicated for 10 min at room temperature in an ultrasonic water bath. Careful visual observations were performed under a hood light using a 7× visual amplifier to determine if precipitates existed on the bottom of the flasks or as a suspension. Additional concentration ranges were tested as needed to determine the maximum solubility limit for each peptidomimetic macrocycle.

### Example 5: X-Ray Co-Crystallography of Peptidomimetic Macrocycles in Complex with MDMX

For co-crystallization with peptide 46 (TABLE 2b), a stoichiometric amount of compound from a 100 mM stock solution in DMSO was added to a zebrafish MDMX protein solution. The solution was allowed to sit overnight at 4° C. before setting up crystallization experiments. Protein (residues 15-129, L46V/V95L) was obtained from an E. coli BL21 (DE3) expression system using the pET15b vector. Cells were grown at 37° C. and induced with 1 mM IPTG at an OD₆₀₀ of 0.7. Cells were allowed to grow an additional 18 hr at 23° C. The protein was purified using Ni-NT Agarose followed by Superdex 75 buffered with 50 mM NaPO₄, pH 8.0, 150 mM NaCl, and 2 mM TCEP, and concentrating to 24 mg/ml. The buffer was exchanged to 20 mM Tris, pH 8.0, 50 mM NaCl, and 2 mM DTT for crystallization experiments. Initial crystals were obtained with the Nextal AMS screen #94, and the final optimized reservoir was 2.6 M AMS, 75 mM Hepes, pH 7.5. Crystals grew routinely as thin plates at 4° C. and were cryo-protected by pulling the crystals through a solution containing concentrated (3.4 M) malonate followed by flash cooling, storage, and shipment in liquid nitrogen.

Data collection was performed at the APS at beamline 31-ID (SGX-CAT) at 100° K and wavelength 0.97929 Å. The beamline was equipped with a Rayonix 225-HE detector. For data collection, crystals were rotated through 180° in 1° increments using 0.8 second exposure times. Data were processed and reduced using Mosflm/scala (CCP4) in space group C2 (unit cell: a=109.2786, b=81.0836, c=30.9058 Å, α=90, β=89.8577, γ=90°). Molecular replacement with program Molrep (CCP4) was performed with the MDMX component of the structure, and two molecules were identified in the asymmetric unit. Initial refinement of just the two molecules of the zebrafish MDMX with program Refmac (CCP4) resulted in an R-factor of 0.3424 (R_{free}=0.3712) and rmsd values for bonds (0.018 Å) and angles (1.698°). The electron densities of the stapled peptide components, starting with Gln¹⁹ and including the entire aliphatic staple, were very clear. Further refinement with CNX using data to 2.3 Å resolution resulted in a model (comprised of 1448 atoms from MDMX, 272 atoms from the stapled peptides and 46 water molecules) that was well refined (R_{f}=0.2601, R_{free}=0.3162, rmsd bonds=0.007 Å and rmsd angles=0.916°).

### Example 6: Circular Dichroism (CD) Analysis of Alpha-Helicity

Peptide solutions were analyzed by CD spectroscopy using a spectropolarimeter. A temperature controller was used to maintain temperature control of the optical cell. Results are expressed as mean molar ellipticity [0] (deg cm² dmol⁻¹) as calculated from the equation [θ]=θobs·MRW/10*l*c where θobs is the observed ellipticity in millidegrees, MRW is the mean residue weight of the peptide (peptide molecular weight/number of residues), l is the optical path length of the cell in centimeters, and c is the peptide concentration in mg/ml. Peptide concentrations were determined by amino acid analysis. Stock solutions of peptides were prepared in benign CD buffer (20 mM phosphoric acid, pH 2). The stock solutions were used to prepare peptide solutions of 0.05 mg/ml in either benign CD buffer or CD buffer with 50% trifluoroethanol (TFE) for analyses in a 10 mm path length cell. Variable wavelength measurements of peptide solutions were scanned at 4° C. from 195 to 250 nm, in 0.2 nm increments, and a scan rate 50 nm per minute. The average of six scans is reported.

TABLE 3 shows CD data for selected peptidomimetic macrocycles:

TABLE 3

| | Molar | Molar | Molar | % Helix | % Helix |
|---|---|---|---|---|---|
| | Ellipticity | Ellipticity | Ellipticity | 50% TFE | benign |
| | Benign | 50% TFE | TFE − Molar | compared | compared |
| | (222 in | (222 in | Ellipticity | to 50% TFE | to 50% TFE |
| SP# | 0% TFE) | 50% TFE) | Benign | parent (CD) | parent (CD) |
| 7 | 124 | −19921.4 | −20045.4 | 137.3 | −0.9 |
| 11 | −398.2 | −16623.4 | 16225.2 | 106.1 | 2.5 |
| 41 | −909 | −21319.4 | 20410.4 | 136 | 5.8 |
| 43 | −15334.5 | −18247.4 | 2912.9 | 116.4 | 97.8 |
| 69 | −102.6 | −21509.7 | −21407.1 | 148.2 | 0.7 |
| 71 | −121.2 | −17957 | −17835.9 | 123.7 | 0.8 |
| 154 | −916.2 | −30965.1 | −30048.9 | 213.4 | 6.3 |
| 230 | −213.2 | −17974 | −17760.8 | 123.9 | 1.5 |
| 233 | −477.9 | −19032.6 | −18554.7 | 131.2 | 3.3 |

### Example 7: Direct Binding Assay MDM2 with Fluorescence Polarization (FP)

The assay was performed according to the following general protocol:

1. Dilute MDM2 (In-house, 41 kD) into FP buffer (High salt buffer-200 mM NaCl, 5 mM CHAPS, pH 7.5) to make 10 μM working stock solution.
2. Add 30 μl of 10 μM of protein stock solution into A1 and B1 well of 96-well black HE microplate (Molecular Devices).
3. Fill in 30 μl of FP buffer into column A2 to A12, B2 to B12, C1 to C12, and D1 to D12.
4. 2 or 3 fold series dilution of protein stock from A1, B1 into A2, B2; A2, B2 to A3, B3; . . . to reach the single digit nM concentration at the last dilution point.
5. Dilute 1 mM (in 100% DMSO) of FAM labeled linear peptide with DMSO to 100 μM (dilution 1:10). Then, dilute from 100 μM to 10 μM with water (dilution 1:10) and then dilute with FP buffer from 10 μM to 40 nM (dilution 1:250). This is the working solution which will be a 10 nM concentration in well (dilution 1:4). Keep the diluted FAM labeled peptide in the dark until use.
6. Add 10 μl of 10 nM of FAM labeled peptide into each well and incubate, and read at different time points. K_{D} with 5-FAM-BaLTFEHYWAQLTS-NH₂ (SEQ ID NO: 943) is ˜13.38 nM.

### Example 8: Competitive Fluorescence Polarization Assay for MDM2

MDM2 (41 kD) was diluted into FP buffer (high-salt buffer-200 mM NaCl, 5 mM CHAPS, pH 7.5) to make a 84 nM (2×) working stock solution. 20 μl of the 84 nM (2×) protein stock solution was added into each well of a 96-well black microplate. 1 mM of FAM-labeled linear peptide (in 100% DMSO) was diluted to 100 μM with DMSO (dilution 1:10). Then, diluted solution was further diluted from 100 μM to 10 μM with water (dilution 1:10), and diluted again with FP buffer from 10 μM to 40 nM (dilution 1:250). The resulting working solution resulted in a 10 nM concentration in each well (dilution 1:4). The diluted FAM-labeled peptides were kept in the dark until use.

Unlabeled peptide dose plates were prepared with FP buffer starting with 1 μM (final) of the peptide. 5-fold serial dilutions were made for 6 points using the following dilution scheme. 10 mM of the solution (in 100% DMSO) with DMSO to 5 mM (dilution 1:2); dilution from 5 mM to 500 μM with H₂O (dilution 1:10); and dilution with FP buffer from 500 μM to 20 μM (dilution 1:25). 5-fold serial dilutions from 4 μM (4×) were made for 6 points. 10 l of the serial diluted unlabeled peptides were transferred to each well, which was filled with 20 μl of 84 nM of protein. 10 μl of 10 nM (4×) of FAM-labeled peptide was added into each well, and the wells were incubated for 3 h before being read.

### Example 9: Direct Binding Assay MDMX with Fluorescence Polarization (FP)

MDMX (40 kD) was diluted into FP buffer (high-salt buffer-200 mM NaCl, 5 mM CHAPS, pH 7.5) to make a 10 μM working stock solution. 30 μl of the 10 μM of protein stock solution was added into the A1 and B1 wells of a 96-well black microplate. 30 μl of FP buffer was added to columns A2 to A12, B2 to B12, C1 to C12, and D1 to D12. 2-fold or 3-fold series dilutions of protein stocks were created from A1, B1 into A2, B2; A2, B2 to A3, B3; . . . to reach the single digit nM concentration at the last dilution point. 1 mM (in 100% DMSO) of a FAM-labeled linear peptide was diluted with DMSO to 100 μM (dilution 1:10). The resulting solution was diluted from 100 μM to 10 μM with water (dilution 1:10), and diluted again with FP buffer from 10 μM to 40 nM (dilution 1:250). The working solution resulted in 10 nM concentration in each well (dilution 1:4). The FAM-labeled peptides were kept in the dark until use. 10 μl of the 10 nM FAM-labeled peptide was added into each well, and the plate was incubated and read at different time points. The K_{D} with 5-FAM-BaLTFEHYWAQLTS-NH₂ (SEQ ID NO: 943) was −51 nM.

### Example 10: Competitive Fluorescence Polarization Assay for MDMX

MDMX (40 kD) was diluted into FP buffer (high-salt buffer 200 mM NaCl, 5 mM CHAPS, pH 7.5) to make a 300 nM (2×) working stock solution. 20 μl of the 300 nM (2×) of protein stock solution was added into each well of 96-well black microplate. 1 mM (in 100% DMSO) of a FAM-labeled linear peptide was diluted with DMSO to a concentration of 100 μM (dilution 1:10). The solution was diluted from 100 μM to 10 μM with water (dilution 1:10), and diluted further with FP buffer from 10 μM to 40 nM (dilution 1:250). The final working solution resulted in a concentration of 10 nM per well (dilution 1:4). The diluted FAM-labeled peptide was kept in the dark until use. An unlabeled peptide dose plate was prepared with FP buffer starting with a concentration of 5 μM (final) of a peptide. 5-fold serial dilutions were prepared for 6 points using the following dilution scheme. 10 mM (in 100% DMSO) of the solution was diluted with DMSO to prepare a 5 mM (dilution 1:2) solution. The solution was diluted from 5 mM to 500 μM with H₂O (dilution 1:10), and diluted further with FP buffer from 500 μM to 20 μM (dilution 1:25). 5-fold serial dilutions from 20 μM (4×) were prepared for 6 points. 10 μl of the serially diluted unlabeled peptides were added to each well, which was filled with 20 μl of the 300 nM protein solution. 10 μl of the 10 nM (4×) FAM-labeled peptide solution was added into each well, and the wells were incubated for 3 h before reading.

Results from EXAMPLE 7-EXAMPLE 10 are shown in TABLE 4. The following scale is used: “+” represents a value greater than 1000 nM, “++” represents a value greater than 100 and less than or equal to 1000 nM, “+++” represents a value greater than 10 nM and less than or equal to 100 nM, and “++++” represents a value of less than or equal to 10 nM.

TABLE 4

| SP# | IC₅₀ (MDM2) | IC₅₀ (MDMX) | Ki (MDM2) | Ki (MDMX) |
|---|---|---|---|---|
| 3 | ++ | ++ | +++ | +++ |
| 4 | +++ | ++ | ++++ | +++ |
| 5 | +++ | ++ | ++++ | +++ |
| 6 | ++ | ++ | +++ | +++ |
| 7 | +++ | +++ | ++++ | +++ |
| 8 | ++ | ++ | +++ | +++ |
| 9 | ++ | ++ | +++ | +++ |
| 10 | ++ | ++ | +++ | +++ |
| 11 | +++ | ++ | ++++ | +++ |
| 12 | + | + | +++ | ++ |
| 13 | ++ | ++ | +++ | ++ |
| 14 | +++ | +++ | ++++ | ++++ |
| 15 | +++ | ++ | +++ | +++ |
| 16 | +++ | +++ | ++++ | +++ |
| 17 | +++ | +++ | ++++ | +++ |
| 18 | +++ | +++ | ++++ | ++++ |
| 19 | ++ | +++ | +++ | +++ |
| 20 | ++ | ++ | +++ | +++ |
| 21 | ++ | +++ | +++ | +++ |
| 22 | +++ | +++ | ++++ | +++ |
| 23 | ++ | ++ | +++ | +++ |
| 24 | +++ | ++ | ++++ | +++ |
| 26 | +++ | ++ | ++++ | +++ |
| 28 | +++ | +++ | ++++ | +++ |
| 30 | ++ | ++ | +++ | +++ |
| 32 | +++ | ++ | ++++ | +++ |
| 38 | + | ++ | ++ | +++ |
| 39 | + | ++ | ++ | ++ |
| 40 | ++ | ++ | ++ | +++ |
| 41 | ++ | +++ | +++ | +++ |
| 42 | ++ | ++ | +++ | ++ |
| 43 | +++ | +++ | ++++ | +++ |
| 45 | +++ | +++ | ++++ | ++++ |
| 46 | +++ | +++ | ++++ | +++ |
| 47 | ++ | ++ | +++ | +++ |
| 48 | ++ | ++ | +++ | +++ |
| 49 | ++ | ++ | +++ | +++ |
| 50 | +++ | ++ | ++++ | +++ |
| 52 | +++ | +++ | ++++ | ++++ |
| 54 | ++ | ++ | +++ | +++ |
| 55 | + | + | ++ | ++ |
| 65 | +++ | ++ | ++++ | +++ |
| 68 | ++ | ++ | +++ | +++ |
| 69 | +++ | ++ | ++++ | +++ |
| 70 | ++ | ++ | ++++ | +++ |
| 71 | +++ | ++ | ++++ | +++ |
| 75 | +++ | ++ | ++++ | +++ |
| 77 | +++ | ++ | ++++ | +++ |
| 80 | +++ | ++ | ++++ | +++ |
| 81 | ++ | ++ | +++ | +++ |
| 82 | ++ | ++ | +++ | +++ |
| 85 | +++ | ++ | ++++ | +++ |
| 99 | ++++ | ++ | ++++ | +++ |
| 100 | ++ | ++ | ++++ | +++ |
| 101 | +++ | ++ | ++++ | +++ |
| 102 | ++ | ++ | ++++ | +++ |
| 103 | ++ | ++ | ++++ | +++ |
| 104 | +++ | ++ | ++++ | +++ |
| 105 | +++ | ++ | ++++ | +++ |
| 106 | ++ | ++ | +++ | +++ |
| 107 | ++ | ++ | +++ | +++ |
| 108 | +++ | ++ | ++++ | +++ |
| 109 | +++ | ++ | ++++ | +++ |
| 110 | ++ | ++ | ++++ | +++ |
| 111 | ++ | ++ | ++++ | +++ |
| 112 | ++ | ++ | +++ | +++ |
| 113 | ++ | ++ | +++ | +++ |
| 114 | +++ | ++ | ++++ | +++ |
| 115 | ++++ | ++ | ++++ | +++ |
| 116 | + | + | ++ | ++ |
| 118 | ++++ | ++ | ++++ | +++ |
| 120 | +++ | ++ | ++++ | +++ |
| 121 | ++++ | ++ | ++++ | +++ |
| 122 | ++++ | ++ | ++++ | +++ |
| 123 | ++++ | ++ | ++++ | +++ |
| 124 | ++++ | ++ | ++++ | +++ |
| 125 | ++++ | ++ | ++++ | +++ |
| 126 | ++++ | ++ | ++++ | +++ |
| 127 | ++++ | ++ | ++++ | +++ |
| 128 | ++++ | ++ | ++++ | +++ |
| 129 | ++++ | ++ | ++++ | +++ |
| 130 | ++++ | ++ | ++++ | +++ |
| 133 | ++++ | ++ | ++++ | +++ |
| 134 | ++++ | ++ | ++++ | +++ |
| 135 | ++++ | ++ | ++++ | +++ |
| 136 | ++++ | ++ | ++++ | +++ |
| 137 | ++++ | ++ | ++++ | +++ |
| 139 | ++++ | ++ | ++++ | +++ |
| 142 | ++++ | +++ | ++++ | +++ |
| 144 | ++++ | ++ | ++++ | +++ |
| 146 | ++++ | ++ | ++++ | +++ |
| 148 | ++++ | ++ | ++++ | +++ |
| 150 | ++++ | ++ | ++++ | +++ |
| 153 | ++++ | +++ | ++++ | +++ |
| 154 | ++++ | +++ | ++++ | ++++ |
| 156 | ++++ | ++ | ++++ | +++ |
| 158 | ++++ | ++ | ++++ | +++ |
| 160 | ++++ | ++ | ++++ | +++ |
| 161 | ++++ | ++ | ++++ | +++ |
| 166 | ++++ | ++ | ++++ | +++ |
| 167 | +++ | ++ | ++++ | ++ |
| 169 | ++++ | +++ | ++++ | +++ |
| 170 | ++++ | ++ | ++++ | +++ |
| 173 | ++++ | ++ | ++++ | +++ |
| 175 | ++++ | ++ | ++++ | +++ |
| 177 | +++ | ++ | ++++ | +++ |
| 180 | +++ | ++ | ++++ | +++ |
| 182 | ++++ | ++ | ++++ | +++ |
| 185 | +++ | + | ++++ | ++ |
| 186 | +++ | ++ | ++++ | +++ |
| 189 | +++ | ++ | ++++ | +++ |
| 192 | +++ | ++ | ++++ | +++ |
| 194 | +++ | ++ | ++++ | ++ |
| 196 | +++ | ++ | ++++ | +++ |
| 197 | ++++ | ++ | ++++ | +++ |
| 199 | +++ | ++ | ++++ | ++ |
| 201 | +++ | ++ | ++++ | ++ |
| 203 | +++ | ++ | ++++ | +++ |
| 204 | +++ | ++ | ++++ | +++ |
| 206 | +++ | ++ | ++++ | +++ |
| 207 | ++++ | ++ | ++++ | +++ |
| 210 | ++++ | ++ | ++++ | +++ |
| 211 | ++++ | ++ | ++++ | +++ |
| 213 | ++++ | ++ | ++++ | +++ |
| 215 | +++ | ++ | ++++ | +++ |
| 217 | ++++ | ++ | ++++ | +++ |
| 218 | ++++ | ++ | ++++ | +++ |
| 221 | ++++ | +++ | ++++ | +++ |
| 227 | ++++ | ++ | ++++ | +++ |
| 230 | ++++ | +++ | ++++ | ++++ |
| 232 | ++++ | ++ | ++++ | +++ |
| 233 | ++++ | +++ | ++++ | +++ |
| 236 | +++ | ++ | ++++ | +++ |
| 237 | +++ | ++ | ++++ | +++ |
| 238 | +++ | +++ | ++++ | +++ |
| 239 | +++ | ++ | +++ | +++ |
| 240 | +++ | ++ | ++++ | +++ |
| 241 | +++ | ++ | ++++ | +++ |
| 242 | +++ | ++ | ++++ | +++ |
| 243 | +++ | +++ | ++++ | +++ |
| 244 | +++ | +++ | ++++ | ++++ |
| 245 | +++ | +++ | ++++ | +++ |
| 246 | +++ | ++ | ++++ | +++ |
| 247 | +++ | +++ | ++++ | +++ |
| 248 | +++ | +++ | ++++ | +++ |
| 249 | +++ | +++ | ++++ | ++++ |
| 250 | ++ | + | ++ | + |
| 252 | ++ | + | ++ | + |
| 254 | +++ | ++ | ++++ | +++ |
| 255 | +++ | +++ | ++++ | +++ |
| 256 | +++ | +++ | ++++ | +++ |
| 257 | +++ | +++ | ++++ | +++ |
| 258 | +++ | ++ | ++++ | +++ |
| 259 | +++ | +++ | ++++ | +++ |
| 260 | +++ | +++ | ++++ | +++ |
| 261 | +++ | ++ | ++++ | +++ |
| 262 | +++ | ++ | ++++ | +++ |
| 263 | +++ | ++ | ++++ | +++ |
| 264 | +++ | +++ | ++++ | +++ |
| 266 | +++ | ++ | ++++ | +++ |
| 267 | +++ | +++ | ++++ | ++++ |
| 270 | ++++ | +++ | ++++ | +++ |
| 271 | ++++ | +++ | ++++ | ++++ |
| 272 | ++++ | +++ | ++++ | ++++ |
| 276 | +++ | +++ | ++++ | ++++ |
| 277 | +++ | +++ | ++++ | ++++ |
| 278 | +++ | +++ | ++++ | ++++ |
| 279 | ++++ | +++ | ++++ | +++ |
| 280 | +++ | ++ | ++++ | +++ |
| 281 | +++ | + | +++ | ++ |
| 282 | ++ | + | +++ | + |
| 283 | +++ | ++ | +++ | ++ |
| 284 | +++ | ++ | ++++ | +++ |
| 289 | +++ | +++ | ++++ | +++ |
| 291 | +++ | +++ | ++++ | ++++ |
| 293 | ++++ | +++ | ++++ | +++ |
| 306 | ++++ | ++ | ++++ | +++ |
| 308 | ++ | ++ | +++ | +++ |
| 310 | +++ | +++ | ++++ | +++ |
| 312 | +++ | ++ | +++ | +++ |
| 313 | ++++ | ++ | ++++ | +++ |
| 314 | ++++ | +++ | ++++ | ++++ |
| 315 | +++ | +++ | ++++ | +++ |
| 316 | ++++ | ++ | ++++ | +++ |
| 317 | +++ | ++ | +++ | +++ |
| 318 | +++ | ++ | +++ | +++ |
| 319 | +++ | ++ | +++ | ++ |
| 320 | +++ | ++ | +++ | ++ |
| 321 | +++ | ++ | ++++ | +++ |
| 322 | +++ | ++ | +++ | ++ |
| 323 | +++ | + | +++ | ++ |
| 328 | +++ | +++ | ++++ | +++ |
| 329 | +++ | +++ | ++++ | +++ |
| 331 | ++++ | +++ | ++++ | ++++ |
| 332 | ++++ | +++ | ++++ | ++++ |
| 334 | ++++ | +++ | ++++ | ++++ |
| 336 | ++++ | +++ | ++++ | ++++ |
| 339 | ++++ | ++ | ++++ | +++ |
| 341 | +++ | +++ | ++++ | ++++ |
| 343 | +++ | +++ | ++++ | ++++ |
| 347 | +++ | +++ | ++++ | +++ |
| 349 | ++++ | +++ | ++++ | ++++ |
| 351 | ++++ | +++ | ++++ | ++++ |
| 353 | ++++ | +++ | ++++ | ++++ |
| 355 | ++++ | +++ | ++++ | ++++ |
| 357 | ++++ | +++ | ++++ | ++++ |
| 359 | ++++ | +++ | ++++ | +++ |
| 360 | ++++ | ++++ | ++++ | ++++ |
| 363 | +++ | +++ | ++++ | ++++ |
| 364 | +++ | +++ | ++++ | ++++ |
| 365 | +++ | +++ | ++++ | ++++ |
| 366 | +++ | +++ | ++++ | +++ |
| 369 | ++ | ++ | +++ | +++ |
| 370 | +++ | +++ | ++++ | +++ |
| 371 | ++ | ++ | +++ | +++ |
| 372 | ++ | ++ | +++ | +++ |
| 373 | +++ | +++ | +++ | +++ |
| 374 | +++ | +++ | ++++ | ++++ |
| 375 | +++ | +++ | ++++ | ++++ |
| 376 | +++ | +++ | ++++ | ++++ |
| 377 | +++ | +++ | ++++ | +++ |
| 378 | +++ | +++ | ++++ | +++ |
| 379 | +++ | +++ | ++++ | +++ |
| 380 | +++ | +++ | ++++ | +++ |
| 381 | +++ | +++ | ++++ | +++ |
| 382 | +++ | +++ | ++++ | ++++ |
| 384 | ++ | + | ++ | + |
| 386 | ++ | + | ++ | + |
| 388 | ++ | +++ | +++ | ++++ |
| 390 | +++ | +++ | ++++ | +++ |
| 392 | +++ | +++ | ++++ | ++++ |
| 394 | ++++ | +++ | ++++ | ++++ |
| 396 | ++++ | ++++ | ++++ | ++++ |
| 398 | +++ | +++ | ++++ | +++ |
| 402 | ++++ | ++++ | ++++ | ++++ |
| 404 | +++ | +++ | ++++ | ++++ |
| 408 | +++ | +++ | ++++ | +++ |
| 410 | ++++ | ++++ | ++++ | ++++ |
| 411 | ++ | + | ++ | + |
| 412 | ++++ | +++ | ++++ | ++++ |
| 415 | ++++ | ++++ | ++++ | ++++ |
| 416 | +++ | +++ | ++++ | +++ |
| 417 | +++ | +++ | ++++ | +++ |
| 418 | ++++ | +++ | ++++ | ++++ |
| 419 | +++ | +++ | +++ | ++++ |
| 421 | ++++ | ++++ | ++++ | ++++ |
| 423 | +++ | +++ | ++++ | +++ |
| 425 | +++ | +++ | +++ | +++ |
| 427 | ++ | ++ | +++ | +++ |
| 432 | ++++ | +++ | ++++ | ++++ |
| 434 | +++ | +++ | ++++ | +++ |
| 435 | ++++ | +++ | ++++ | ++++ |
| 437 | +++ | +++ | ++++ | +++ |
| 439 | ++++ | +++ | ++++ | ++++ |
| 441 | ++++ | ++++ | ++++ | ++++ |
| 443 | +++ | +++ | ++++ | +++ |
| 445 | +++ | ++ | ++++ | +++ |
| 446 | +++ | + | ++++ | + |
| 447 | ++ | + | ++ | + |
| 551 | N/A | N/A | ++++ | +++ |
| 555 | N/A | N/A | ++++ | +++ |
| 556 | N/A | N/A | ++++ | +++ |
| 557 | N/A | N/A | +++ | +++ |
| 558 | N/A | N/A | +++ | +++ |
| 559 | N/A | N/A | +++ | +++ |
| 560 | N/A | N/A | + | + |
| 561 | N/A | N/A | ++++ | +++ |
| 562 | N/A | N/A | +++ | +++ |
| 563 | N/A | N/A | +++ | +++ |
| 564 | N/A | N/A | ++++ | +++ |
| 565 | N/A | N/A | +++ | +++ |
| 566 | N/A | N/A | ++++ | +++ |
| 567 | N/A | N/A | ++++ | +++ |
| 568 | N/A | N/A | ++++ | ++++ |
| 569 | N/A | N/A | ++++ | +++ |
| 570 | N/A | N/A | ++++ | +++ |
| 571 | N/A | N/A | ++++ | +++ |
| 572 | N/A | N/A | +++ | +++ |
| 573 | N/A | N/A | +++ | +++ |
| 574 | N/A | N/A | ++++ | +++ |
| 575 | N/A | N/A | ++++ | +++ |
| 576 | N/A | N/A | ++++ | +++ |
| 577 | N/A | N/A | ++++ | +++ |
| 578 | N/A | N/A | ++++ | +++ |
| 585 | N/A | N/A | +++ | +++ |
| 586 | N/A | N/A | ++++ | +++ |
| 587 | N/A | N/A | ++++ | ++++ |
| 589 | N/A | N/A | ++++ | |
| 594 | N/A | N/A | ++++ | ++++ |
| 596 | N/A | N/A | ++++ | +++ |
| 597 | N/A | N/A | ++++ | +++ |
| 598 | N/A | N/A | ++++ | +++ |
| 600 | N/A | N/A | ++++ | ++++ |
| 602 | N/A | N/A | ++++ | ++++ |
| 603 | N/A | N/A | ++++ | ++++ |
| 604 | N/A | N/A | +++ | +++ |
| 608 | N/A | N/A | ++++ | +++ |
| 609 | N/A | N/A | ++++ | +++ |
| 610 | N/A | N/A | ++++ | +++ |
| 611 | N/A | N/A | ++++ | +++ |
| 612 | N/A | N/A | ++++ | +++ |
| 613 | N/A | N/A | ++++ | +++ |
| 615 | N/A | N/A | ++++ | ++++ |
| 433 | N/A | N/A | ++++ | +++ |
| 686 | N/A | N/A | ++++ | +++ |
| 687 | N/A | N/A | ++ | ++ |
| 595 | N/A | N/A | + | N/A |
| 665 | N/A | N/A | +++ | N/A |
| 708 | N/A | N/A | +++ | +++ |
| 710 | N/A | N/A | +++ | +++ |
| 711 | N/A | N/A | +++ | ++ |
| 712 | N/A | N/A | ++++ | ++++ |
| 713 | N/A | N/A | ++++ | ++++ |
| 716 | N/A | N/A | ++++ | ++++ |
| 765 | + | + | | |
| 766 | +++ | + | | |
| 752 | ++ | + | | |
| 753 | +++ | + | | |
| 754 | ++ | + | | |
| 755 | ++++ | + | | |
| 756 | +++ | + | | |
| 757 | ++++ | + | | |
| 758 | +++ | + | | |

### Example 11: Competition Binding ELISA Assay for MDM2 and MDMX

p53-His6 protein (30 nM/well) was coated overnight at room temperature in the wells of 96-well plates. On the day of the experiment, the plates were washed with 1×PBS-Tween 20 (0.05%) using an automated ELISA plate washer, and blocked with ELISA microwell blocking buffer for 30 minutes at room temperature. The excess blocking agent was washed off by washing the plates with 1×PBS-Tween 20 (0.05%). The peptides were diluted from 10 mM DMSO stock solutions to 500 μM working stock solutions using sterile water. Further dilutions were made in 0.5% DMSO to keep the concentration of DMSO constant across the samples. The peptide solutions were added to the wells at 2× the desired concentrations in 50 μL volumes, followed by addition of diluted GST-MDM2 or GST-HMDX protein (final concentration: 10 nM). The samples were incubated at room temperature for 2 h, and the plates were washed with PBS-Tween 20 (0.05%) prior to adding 100 μL of HRP-conjugated anti-GST antibody diluted to 0.5 μg/ml in HRP-stabilizing buffer. The plates were incubated with a detection antibody for 30 min, and the plates were washed and incubated with 100 μL per well of TMB-E substrate solution for up to 30 minutes. The reactions were stopped using 1M HCL, and absorbance was measured at 450 nm using a micro plate reader. The data were analyzed using Graph Pad PRISM software.

### Example 12: Cell Viability Assay

Cells were trypsinized, counted, and seeded at pre-determined densities in 96-well plates one day prior to conducting the cell viability assay. The following cell densities were used for each cell line: SJSA-1: 7500 cells/well; RKO: 5000 cells/well; RKO-E6: 5000 cells/well; HCT-116: 5000 cells/well; SW-480: 2000 cells/well; and MCF-7: 5000 cells/well. On the day of cell viability assay, the media was replaced with fresh media containing 11% FBS (assay media) at room temperature. 180 μL of the assay media was added to each well. Control wells were prepared with no cells, and the control wells received 200 μL of media.

Peptide dilutions were made at room temperature, and the diluted peptide solutions were added to the cells at room temperature. 10 mM stock solutions of the peptides were prepared in DMSO. The stock solutions were serially diluted using a 1:3 dilution scheme to obtain 10 mM, 3.3 mM, 1.1 mM, 0.33 mM, 0.11 mM, 0.03 mM, and 0.01 mM solutions in DMSO. The serially DMSO-diluted peptides were diluted 33.3 times using sterile water, resulting in a range of 10× working stock solutions. A DMSO/sterile water (3% DMSO) solution was prepared for use in the control well. The working stock solution concentrations ranges were 300 μM, 100 μM, 30 μM, 10 μM, 3 μM, 1 μM, 0.3 μM, and 0 μM. The solutions were mixed well at each dilution step using a multichannel pipette.

Row H of the plate contained the controls. Wells H1-H3 received 20 μL of assay media. Rows H4-H9 received 20 μL of the 3% DMSO-water vehicle. Wells H10-H12 received media alone control with no cells. The MDM2 small molecule inhibitor Nutlin-3a (10 mM) was used as a positive control. Nutlin-3a was diluted using the same dilution scheme used for the peptides.

20 μL of a 10× concentration peptide stock solution was added to the appropriate well to achieve the final concentration in 200 μL in each well. For example, 20 μL of 300 μM peptide solution+180 μL of cells in media=30 μM final concentration in 200 μL volume in wells. The solution was mixed gently a few times using a pipette. The final concentration range was 30 μM, 10 μM, 3 μM, 1 μM, 0.3 μM, 0.1 μM, 0.03 μM, and 0 μM. Further dilutions were used for potent peptides. Controls included wells that received no peptides, but contained the same concentration of DMSO as the wells containing peptides and wells containing no cells. The plates were incubated for 72 hours at 37° C. in a humidified 5% CO₂ atmosphere.

The viability of the cells was determined using MTT reagent. The viability of SJSA-1, RKO, RKO-E6, HCT-116 cells was determined on day 3. The viability of MCF-7 cells was determined on day 5. The viability of SW-480 cells was determined on on day 6. At the end of the designated incubation time, the plates were cooled to room temperature. 80 μL of assay media was removed from each well. 15 μL of thawed MTT reagent was then added to each well. The plate was incubated for 2 h at 37° C. in a humidified 5% CO₂ atmosphere. 100 μL of the solubilization reagent was added to each well. The plates were incubated with agitation for 1 h at room temperature, and read using a multiplate reader for absorbance at 570 nM. Cell viability was analyzed against the DMSO controls.

Results from cell viability assays are shown in TABLE 5 and TABLE 6. “+” represents a value greater than 30 μM, “++” represents a value greater than 15 μM and less than or equal to 30 M, “+++” represents a value greater than 5 μM and less than or equal to 15 μM, and “++++” represents a value of less than or equal to 5 μM. “IC₅₀ ratio” represents the ratio of average IC₅₀ in p53+/+ cells relative to average IC₅₀ in p53−/− cells.

TABLE 5

| | SJSA-1 EC50 |
|---|---|
| SP# | (72 h) |
| 3 | +++ |
| 4 | +++ |
| 5 | ++++ |
| 6 | ++ |
| 7 | ++++ |
| 8 | +++ |
| 9 | +++ |
| 10 | +++ |
| 11 | ++++ |
| 12 | ++ |
| 13 | +++ |
| 14 | + |
| 15 | ++ |
| 16 | + |
| 17 | + |
| 18 | + |
| 19 | ++ |
| 20 | + |
| 21 | + |
| 22 | + |
| 24 | +++ |
| 26 | ++++ |
| 28 | + |
| 29 | + |
| 30 | + |
| 32 | ++ |
| 38 | + |
| 39 | + |
| 40 | + |
| 41 | + |
| 42 | + |
| 43 | ++ |
| 45 | + |
| 46 | + |
| 47 | + |
| 48 | + |
| 49 | +++ |
| 50 | ++++ |
| 52 | + |
| 54 | + |
| 55 | + |
| 65 | ++++ |
| 68 | ++++ |
| 69 | ++++ |
| 70 | ++++ |
| 71 | ++++ |
| 72 | ++++ |
| 74 | ++++ |
| 75 | ++++ |
| 77 | ++++ |
| 78 | ++ |
| 80 | ++++ |
| 81 | +++ |
| 82 | +++ |
| 83 | +++ |
| 84 | + |
| 85 | +++ |
| 99 | ++++ |
| 102 | +++ |
| 103 | +++ |
| 104 | +++ |
| 105 | +++ |
| 108 | +++ |
| 109 | +++ |
| 110 | +++ |
| 111 | ++ |
| 114 | ++++ |
| 115 | ++++ |
| 118 | ++++ |
| 120 | ++++ |
| 121 | ++++ |
| 122 | ++++ |
| 123 | ++++ |
| 124 | +++ |
| 125 | ++++ |
| 126 | ++++ |
| 127 | ++++ |
| 128 | +++ |
| 129 | ++ |
| 130 | ++++ |
| 131 | +++ |
| 132 | ++++ |
| 133 | +++ |
| 134 | +++ |
| 135 | +++ |
| 136 | ++ |
| 137 | +++ |
| 139 | ++++ |
| 142 | +++ |
| 144 | ++++ |
| 147 | ++++ |
| 148 | ++++ |
| 149 | ++++ |
| 150 | ++++ |
| 152 | +++ |
| 153 | ++++ |
| 154 | ++++ |
| 155 | ++ |
| 156 | +++ |
| 157 | +++ |
| 158 | +++ |
| 160 | ++++ |
| 161 | ++++ |
| 162 | +++ |
| 163 | +++ |
| 166 | ++ |
| 167 | +++ |
| 168 | ++ |
| 169 | ++++ |
| 170 | ++++ |
| 171 | ++ |
| 173 | +++ |
| 174 | ++++ |
| 175 | +++ |
| 176 | +++ |
| 177 | ++++ |
| 179 | +++ |
| 180 | +++ |
| 181 | +++ |
| 182 | ++++ |
| 183 | ++++ |
| 184 | +++ |
| 185 | +++ |
| 186 | ++ |
| 188 | ++ |
| 190 | ++++ |
| 192 | +++ |
| 193 | ++ |
| 194 | + |
| 195 | ++++ |
| 196 | ++++ |
| 197 | ++++ |
| 198 | ++ |
| 199 | +++ |
| 200 | +++ |
| 201 | ++++ |
| 202 | +++ |
| 203 | ++++ |
| 204 | ++++ |
| 205 | ++ |
| 206 | ++ |
| 207 | +++ |
| 208 | +++ |
| 209 | ++++ |
| 210 | +++ |
| 211 | ++++ |
| 213 | ++++ |
| 214 | ++++ |
| 215 | ++++ |
| 216 | ++++ |
| 217 | ++++ |
| 218 | ++++ |
| 219 | ++++ |
| 220 | +++ |
| 221 | ++++ |
| 222 | +++ |
| 223 | ++++ |
| 224 | ++ |
| 225 | +++ |
| 226 | ++ |
| 227 | +++ |
| 228 | ++++ |
| 229 | ++++ |
| 230 | ++++ |
| 231 | ++++ |
| 232 | ++++ |
| 233 | ++++ |
| 234 | ++++ |
| 235 | ++++ |
| 236 | ++++ |
| 237 | ++++ |
| 238 | ++++ |
| 239 | +++ |
| 240 | ++ |
| 241 | +++ |
| 242 | ++++ |
| 243 | ++++ |
| 244 | ++++ |
| 245 | ++++ |
| 246 | +++ |
| 247 | ++++ |
| 248 | ++++ |
| 249 | ++++ |
| 250 | ++ |
| 251 | + |
| 252 | + |
| 253 | + |
| 254 | +++ |
| 255 | +++ |
| 256 | ++ |
| 257 | +++ |
| 258 | +++ |
| 259 | ++ |
| 260 | ++ |
| 261 | ++ |
| 262 | +++ |
| 263 | ++ |
| 264 | ++++ |
| 266 | +++ |
| 267 | ++++ |
| 270 | ++ |
| 271 | ++ |
| 272 | ++ |
| 276 | ++ |
| 277 | ++ |
| 278 | ++ |
| 279 | ++++ |
| 280 | +++ |
| 281 | ++ |
| 282 | ++ |
| 283 | ++ |
| 284 | ++++ |
| 289 | ++++ |
| 290 | +++ |
| 291 | ++++ |
| 292 | ++++ |
| 293 | ++++ |
| 294 | ++++ |
| 295 | +++ |
| 296 | ++++ |
| 297 | +++ |
| 298 | ++++ |
| 300 | ++++ |
| 301 | ++++ |
| 302 | ++++ |
| 303 | ++++ |
| 304 | ++++ |
| 305 | ++++ |
| 306 | ++++ |
| 307 | +++ |
| 308 | ++++ |
| 309 | +++ |
| 310 | ++++ |
| 312 | ++++ |
| 313 | ++++ |
| 314 | ++++ |
| 315 | ++++ |
| 316 | ++++ |
| 317 | ++++ |
| 318 | ++++ |
| 319 | ++++ |
| 320 | ++++ |
| 321 | ++++ |
| 322 | ++++ |
| 323 | ++++ |
| 324 | ++++ |
| 326 | ++++ |
| 327 | ++++ |
| 328 | ++++ |
| 329 | ++++ |
| 330 | ++++ |
| 331 | ++++ |
| 332 | ++++ |
| 333 | ++ |
| 334 | +++ |
| 335 | ++++ |
| 336 | ++++ |
| 337 | ++++ |
| 338 | ++++ |
| 339 | ++++ |
| 340 | ++++ |
| 341 | ++++ |
| 342 | ++++ |
| 343 | ++++ |
| 344 | ++++ |
| 345 | ++++ |
| 346 | ++++ |
| 347 | ++++ |
| 348 | ++++ |
| 349 | ++++ |
| 350 | ++++ |
| 351 | ++++ |
| 352 | ++++ |
| 353 | ++++ |
| 355 | ++++ |
| 357 | ++++ |
| 358 | ++++ |
| 359 | ++++ |
| 360 | ++++ |
| 361 | +++ |
| 362 | ++++ |
| 363 | ++++ |
| 364 | ++++ |
| 365 | +++ |
| 366 | ++++ |
| 367 | ++++ |
| 368 | + |
| 369 | ++++ |
| 370 | ++++ |
| 371 | ++++ |
| 372 | +++ |
| 373 | +++ |
| 374 | ++++ |
| 375 | ++++ |
| 376 | ++++ |
| 377 | ++++ |
| 378 | ++++ |
| 379 | ++++ |
| 380 | ++++ |
| 381 | ++++ |
| 382 | ++++ |
| 386 | +++ |
| 388 | ++ |
| 390 | ++++ |
| 392 | +++ |
| 394 | +++ |
| 396 | +++ |
| 398 | +++ |
| 402 | +++ |
| 404 | +++ |
| 408 | ++++ |
| 410 | +++ |
| 411 | +++ |
| 412 | + |
| 421 | +++ |
| 423 | ++++ |
| 425 | ++++ |
| 427 | ++++ |
| 434 | +++ |
| 435 | ++++ |
| 436 | ++++ |
| 437 | ++++ |
| 438 | ++++ |
| 439 | ++++ |
| 440 | ++++ |
| 441 | ++++ |
| 442 | ++++ |
| 443 | ++++ |
| 444 | +++ |
| 445 | ++++ |
| 449 | ++++ |
| 551 | ++++ |
| 552 | ++++ |
| 554 | + |
| 555 | ++++ |
| 586 | ++++ |
| 587 | ++++ |
| 588 | ++++ |
| 589 | +++ |
| 432 | ++++ |
| 672 | + |
| 673 | ++ |
| 682 | + |
| 686 | + |
| 557 | ++++ |
| 558 | ++++ |
| 560 | + |
| 561 | ++++ |
| 562 | ++++ |
| 563 | ++++ |
| 564 | ++++ |
| 566 | ++++ |
| 567 | ++++ |
| 568 | +++ |
| 569 | ++++ |
| 571 | ++++ |
| 572 | ++++ |
| 573 | ++++ |
| 574 | ++++ |
| 575 | ++++ |
| 576 | ++++ |
| 577 | ++++ |
| 578 | ++++ |
| 585 | ++++ |
| 687 | + |
| 662 | ++++ |
| 663 | ++++ |
| 553 | +++ |
| 559 | ++++ |
| 579 | ++++ |
| 581 | ++++ |
| 582 | ++ |
| 582 | ++++ |
| 584 | +++ |
| 675 | ++++ |
| 676 | ++++ |
| 677 | + |
| 679 | ++++ |
| 700 | +++ |
| 704 | +++ |
| 591 | + |
| 706 | ++ |
| 695 | ++ |
| 595 | ++++ |
| 596 | ++++ |
| 597 | +++ |
| 598 | +++ |
| 599 | ++++ |
| 600 | ++++ |
| 601 | +++ |
| 602 | +++ |
| 603 | +++ |
| 604 | +++ |
| 606 | ++++ |
| 607 | ++++ |
| 608 | ++++ |
| 610 | ++++ |
| 611 | ++++ |
| 612 | ++++ |
| 613 | +++ |
| 614 | +++ |
| 615 | ++++ |
| 618 | ++++ |
| 619 | ++++ |
| 707 | ++++ |
| 620 | ++++ |
| 621 | ++++ |
| 622 | ++++ |
| 623 | ++++ |
| 624 | ++++ |
| 625 | ++++ |
| 626 | +++ |
| 631 | ++++ |
| 633 | ++++ |
| 634 | ++++ |
| 635 | +++ |
| 636 | +++ |
| 638 | + |
| 641 | +++ |
| 665 | ++++ |
| 708 | ++++ |
| 709 | +++ |
| 710 | + |
| 711 | ++++ |
| 712 | ++++ |
| 713 | ++++ |
| 714 | +++ |
| 715 | +++ |
| 716 | ++++ |
| 765 | + |
| 753 | + |
| 754 | + |
| 755 | + |
| 756 | + |
| 757 | ++++ |
| 758 | +++ |

TABLE 6

| | | | | SW480 | |
|---|---|---|---|---|---|
| | HCT-116 EC50 | RKO EC₅₀ | RKO-E6 EC₅₀ | EC50 | IC₅₀ |
| SP# | (72 h) | (72 h) | (72 h) | (6 days) | Ratio |
| 4 | ++++ | ++++ | +++ | ++++ | |
| 5 | ++++ | ++++ | +++ | ++++ | |
| 7 | ++++ | ++++ | +++ | ++++ | |
| 10 | ++++ | +++ | +++ | +++ | |
| 11 | ++++ | ++++ | ++ | +++ | |
| 50 | ++++ | ++++ | ++ | +++ | |
| 65 | +++ | +++ | +++ | +++ | |
| 69 | ++++ | ++++ | + | ++++ | |
| 70 | ++++ | ++++ | ++ | +++ | |
| 71 | ++++ | ++++ | +++ | +++ | |
| 81 | +++ | +++ | +++ | +++ | |
| 99 | ++++ | ++++ | +++ | ++++ | |
| 109 | ++++ | ++++ | ++ | +++ | |
| 114 | | +++ | + | +++ | |
| 115 | | +++ | + | +++ | 1-29 |
| 118 | +++ | ++++ | + | ++++ | |
| 120 | ++++ | ++++ | + | ++++ | |
| 121 | ++++ | ++++ | + | ++++ | |
| 122 | | +++ | + | +++ | 1-29 |
| 125 | +++ | +++ | + | + | |
| 126 | + | + | + | + | |
| 148 | | ++ | + | + | |
| 150 | | ++ | + | + | |
| 153 | +++ | | + | | |
| 154 | +++ | +++ | + | + | 30-49 |
| 158 | + | + | + | + | |
| 160 | +++ | + | + | + | 1-29 |
| 161 | +++ | + | + | + | |
| 175 | + | + | + | + | |
| 196 | ++++ | ++++ | +++ | ++++ | |
| 219 | ++++ | +++ | + | + | 1-29 |
| 233 | ++++ | | | | |
| 237 | ++++ | | + | + | |
| 238 | ++++ | | + | + | |
| 243 | ++++ | | + | + | |
| 244 | ++++ | | + | + | ≥50 |
| 245 | ++++ | | + | + | |
| 247 | ++++ | | + | + | |
| 249 | ++++ | ++++ | + | + | ≥50 |
| 255 | ++++ | | + | | |
| 291 | | | + | | |
| 293 | +++ | | + | | |
| 303 | +++ | | + | | 1-29 |
| 305 | | | + | | |
| 306 | ++++ | | + | | |
| 310 | ++++ | | + | | |
| 312 | ++++ | | | | |
| 313 | ++++ | | ++ | | |
| 314 | | | + | | |
| 315 | ++++ | ++++ | ++ | ++++ | ≥50 |
| 316 | ++++ | ++++ | + | +++ | ≥50 |
| 317 | +++ | | + | ++ | |
| 321 | ++++ | | + | | |
| 324 | +++ | | + | | |
| 325 | +++ | | | | |
| 326 | +++ | | + | | |
| 327 | +++ | | + | | |
| 328 | +++ | | ++ | | |
| 329 | ++++ | | + | | |
| 330 | | | + | | |
| 331 | ++++ | ++++ | + | + | ≥50 |
| 338 | ++++ | ++++ | ++ | +++ | |
| 341 | +++ | ++ | + | + | |
| 343 | +++ | | + | + | |
| 346 | ++++ | | + | + | |
| 347 | +++ | | + | + | |
| 349 | ++++ | +++ | + | + | 30-49 |
| 350 | ++++ | | + | + | |
| 351 | ++++ | +++ | + | + | 30-49 |
| 353 | ++ | ++ | + | + | |
| 355 | ++++ | ++ | + | + | 1-29 |
| 357 | ++++ | ++++ | + | + | |
| 358 | ++++ | ++ | + | + | |
| 359 | ++++ | ++ | + | + | |
| 367 | ++++ | | + | + | 30-49 |
| 386 | ++++ | ++++ | ++++ | ++++ | |
| 388 | ++ | ++ | + | +++ | 1-29 |
| 390 | ++++ | ++++ | +++ | ++++ | |
| 435 | +++ | ++ | + | | |
| 436 | ++++ | ++++ | ++ | | |
| 437 | ++++ | ++++ | ++ | ++++ | 30-49 |
| 440 | ++ | ++ | + | | |
| 442 | ++++ | ++++ | ++ | | |
| 444 | ++++ | ++++ | +++ | | |
| 445 | ++++ | +++ | + | + | ≥50 |
| 555 | | | | | ≥50 |
| 557 | | | | | ≥50 |
| 558 | | | | | 30-49 |
| 562 | | | | | 30-49 |
| 564 | | | | | 30-49 |
| 566 | | | | | 30-49 |
| 567 | | | | | ≥50 |
| 572 | | | | | ≥50 |
| 573 | | | | | 30-49 |
| 578 | | | | | 30-49 |
| 662 | | | | | ≥50 |
| 379 | | | | | 1-29 |
| 375 | | | | | 1-29 |
| 559 | | | | | ≥50 |
| 561 | | | | | 1-29 |
| 563 | | | | | 1-29 |
| 568 | | | | | 1-29 |
| 569 | | | | | 1-29 |
| 571 | | | | | 1-29 |
| 574 | | | | | 1-29 |
| 575 | | | | | 1-29 |
| 576 | | | | | 1-29 |
| 577 | | | | | 30-49 |
| 433 | | | | | 1-29 |
| 551 | | | | | 30-49 |
| 553 | | | | | 1-29 |
| 710 | | | | + | |
| 711 | | | | + | |
| 712 | | | | ++ | |
| 713 | | | | ++ | |
| 714 | | | | +++ | |
| 715 | | | | +++ | |
| 716 | | | | + | |

### Example 13: p21 ELISA Assay

SJSA-1 cells were trypsinized, counted, and seeded at a density of 7500 cells/100 μL/well in 96-well plates one day prior to running the assay. On the day of the assay, the media was replaced with fresh RPMI-11% FBS assay media. 90 μL of the assay media was added to each well. The control wells contained no cells and received 100 μL of the assay media.

10 mM stock solutions of the peptides were prepared in DMSO. The stock solutions were serially diluted using a 1:3 dilution scheme to obtain 10 mM, 3.3 mM, 1.1 mM, 0.33 mM, 0.11 mM, 0.03 mM, and 0.01 mM solutions in DMSO. The solutions were serially diluted 33.3 times using sterile water to provide a range of 10× working stock solutions. A DMSO/sterile water (3% DMSO) solution was prepared for use in the control wells. The working stock solution concentration range was 300 μM, 100 μM, 30 μM, 10 μM, 3 μM, 1 μM, 0.3 μM, and 0 μM. Each solution was mixed well at each dilution step using a multichannel pipette. Row H contained the control wells. Wells H1-H3 received 10 μL of the assay media. Wells H4-H9 received 10 μL of the 3% DMSO-water solution. Wells H10-H12 received media alone and contained no cells. The MDM2 small molecule inhibitor Nutlin-3a (10 mM) was used as a positive control. Nutlin-3a was diluted using the same dilution scheme used for the peptides.

10 μL of a 10× peptide solution was added to the appropriate well to achieve a final concentration in a volume of 100 μL. For example, 10 μL of 300 μM peptide+90 μL of cells in media=30 μM final concentration in 100 μL volume in wells. The final concentration range used was 30 μM, 10 μM, 3 μM, 1 μM, 0.3 μM, and 0 μM. Control wells included wells that did not receive peptides but contained the same concentration of DMSO as the wells containing the peptides and wells containing no cells.

20 h after incubation, the media was aspirated from the wells. The cells were washed with 1×PBS (without Ca⁺⁺/Mg⁺⁺) and lysed in 60 μL of 1× cell lysis buffer (10× buffer diluted to 1× and supplemented with protease inhibitors and phosphatase inhibitors) on ice for 30 min. The plates were centrifuged at 5000 rpm at 4° C. for 8 min. The clear supernatants were collected and frozen at −80° C. until further use. The total protein contents of the lysates were measured using a BCA protein detection kit and BSA standards. Each well provided about 6-7 μg of protein. 50 μL of the lysate was used per well to set up the p21 ELISA assay. For the human total p21 ELISA assay, 50 μL of lysate was used for each well, and each well was set up in triplicate.

### Example 14: Caspase 3 Detection Assay

SJSA-1 cells were trypsinized, counted, and seeded at a density of 7500 cells/100 μL/well in 96-well plates one day prior to conducting the assay. One the day of the assay, the media was replaced with fresh RPMI-11% FBS assay media. 180 μL of the assay media was added to each well. Control wells contained no cells, and received 200 μL of the assay media.

10 mM stock solutions of the peptides were prepared in DMSO. The stock solutions were serially diluted using a 1:3 dilution scheme to obtain 10 mM, 3.3 mM, 1.1 mM, 0.33 mM, 0.11 mM, 0.03 mM, and 0.01 mM solutions in DMSO. The solutions were serially diluted 33.3 times using sterile water to provide a range of 10× working stock solutions. A DMSO/sterile water (3% DMSO) solution was prepared for use in the control wells. The working stock solution concentration range was 300 μM, 100 μM, 30 μM, 10 μM, 3 μM, 1 μM, 0.3 μM, and 0 μM. Each well was mixed well at each dilution step using a multichannel pipette. 20 μL of the 10× working stock solutions were added to the appropriate wells. Row H of the plates had control wells. Wells H1-H3 received 20 μL of the assay media. Wells H4-H9 received 20 μL of the 3% DMSO-water solutions. Wells H10-H12 received media and had no cells. The MDM2 small molecule inhibitor Nutlin-3a (10 mM) was used as a positive control. Nutlin-3a was diluted using the same dilution scheme as the peptides.

10 μL of the 10× stock solutions were added to the appropriate wells to achieve the final concentrations in a total volume of 100 μL. For example, 10 μL of 300 μM peptide+90 μL of cells in media=30 μM final concentration in 100 μL volume in wells. The final concentration range used was 30 μM, 10 μM, 3 μM, 1 μM, 0.3 μM, and 0 μM. Control wells contained no peptides but contained the same concentration of DMSO as the wells containing the peptides and well containing no cells. 48 h after incubation, 80 μL of the media was aspirated from each well. 100 μL of Caspase 3/7Glo assay reagent was added to each well. The plates were incubated with gentle shaking for 1 h at room temperature and read using a multi-plate reader for luminescence. Data were analyzed as Caspase 3 activation over DMSO-treated cells. Results from EXAMPLE 13 and EXAMPLE 14 are shown in TABLE 7.

TABLE 7

| | caspase | caspase | caspase | caspase | caspase | p21 | p21 | p21 | p21 | p21 |
|---|---|---|---|---|---|---|---|---|---|---|
| SP# | 0.3 μM | 1 μM | 3 μM | 10 μM | 30 μM | 0.3 μM | 1 μM | 3 μM | 10 μM | 30 μM |
| 4 | | | 9 | 37 | 35 | | | 317 | 3049 | 3257 |
| 7 | 0.93 | 1.4 | 5.08 | 21.7 | 23.96 | | 18 | 368 | 1687 | 2306 |
| 8 | | | 1 | 19 | 25 | | | 34 | 972 | 2857 |
| 10 | 1 | | 1 | 17 | 32 | | 10 | 89 | 970 | 2250 |
| 11 | 1 | | 5 | 23 | 33.5 | | 140 | 350 | 2075.5 | 3154 |
| 26 | 1 | | 1 | 3 | 14 | | | | | |
| 50 | | | 8 | 29 | 29 | | 44 | 646 | 1923 | 1818 |
| 65 | 1 | | 6 | 28 | 34 | −69 | −24 | 122 | 843 | 1472 |
| 69 | 4.34 | 9.51 | 16.39 | 26.59 | 26.11 | 272 | 458.72 | 1281.39 | 2138.88 | 1447.22 |
| 70 | | | 1 | 9 | 26 | | −19 | 68 | 828 | 1871 |
| 71 | 0.95 | 1.02 | 3.68 | 14.72 | 23.52 | | 95 | 101 | 1204 | 2075 |
| 72 | 1 | | 1 | 4 | 10 | −19 | 57 | 282 | 772 | 1045 |
| 77 | 1 | | 2 | 19 | 23 | | | | | |
| 80 | 1 | | 2 | 13 | 20 | | | | | |
| 81 | 1 | | 1 | 6 | 21 | | 0 | 0 | 417 | 1649 |
| 99 | 1 | | 7 | 31 | 33 | −19 | 117 | 370 | 996 | 1398 |
| 109 | | | 4 | 16 | 25 | | 161 | 445 | 1221 | 1680 |
| 114 | 1 | | 6 | 28 | 34 | −21 | 11 | 116 | 742 | 910 |
| 115 | 1 | | 10 | 26 | 32 | −10 | 36 | 315 | 832 | 1020 |
| 118 | 1 | | 2 | 18 | 27 | −76 | −62 | −11 | 581 | 1270 |
| 120 | 2 | | 11 | 20 | 30 | −4 | 30 | 164 | 756 | 1349 |
| 121 | 1 | | 5 | 19 | 30 | 9 | 33 | 81 | 626 | 1251 |
| 122 | 1 | | 2 | 15 | 30 | −39 | −18 | 59 | 554 | 1289 |
| 123 | 1 | | 1 | 6 | 14 | | | | | |
| 125 | 1 | | 3 | 9 | 29 | 50 | 104 | 196 | 353 | 1222 |
| 126 | 1 | | 1 | 6 | 30 | −47 | −10 | 90 | 397 | 1443 |
| 127 | 1 | | 1 | 4 | 13 | | | | | |
| 130 | 1 | | 2 | 6 | 17 | | | | | |
| 139 | 1 | | 2 | 9 | 18 | | | | | |
| 142 | 1 | | 2 | 15 | 20 | | | | | |
| 144 | 1 | | 4 | 10 | 16 | | | | | |
| 148 | 1 | | 11 | 23 | 31 | −23 | 55 | 295 | 666 | 820 |
| 149 | 1 | | 2 | 4 | 10 | 35 | 331 | 601 | 1164 | 1540 |
| 150 | 2 | | 11 | 19 | 35 | −37 | 24 | 294 | 895 | 906 |
| 153 | 2 | | 10 | 15 | 20 | | | | | |
| 154 | 2.68 | 4 | 13.93 | 19.86 | 30.14 | 414.04 | 837.45 | 1622.42 | 2149.51 | 2156.98 |
| 158 | 1 | | 1.67 | 5 | 16.33 | −1.5 | 95 | 209.5 | 654 | 1665.5 |
| 160 | 2 | | 10 | 16 | 31 | −43 | 46 | 373 | 814 | 1334 |
| 161 | 2 | | 8 | 14 | 22 | 13 | 128 | 331 | 619 | 1078 |
| 170 | 1 | | 1 | 16 | 20 | | | | | |
| 175 | 1 | | 5 | 12 | 21 | −65 | 1 | 149 | 543 | 1107 |
| 177 | 1 | | 1 | 8 | 20 | | | | | |
| 183 | 1 | | 1 | 4 | 8 | −132 | −119 | −14 | 1002 | 818 |
| 196 | 1 | | 4 | 33 | 26 | −49 | −1 | 214 | 1715 | 687 |
| 197 | 1 | | 1 | 10 | 20 | | | | | |
| 203 | 1 | | 3 | 12 | 10 | 77 | 329 | 534 | 1805 | 380 |
| 204 | 1 | | 4 | 10 | 10 | 3 | 337 | 928 | 1435 | 269 |
| 218 | 1 | | 2 | 8 | 18 | | | | | |
| 219 | 1 | | 5 | 17 | 34 | 28 | 53 | 289 | 884 | 1435 |
| 221 | 1 | | 3 | 6 | 12 | 127 | 339 | 923 | 1694 | 1701 |
| 223 | 1 | | 1 | 5 | 18 | | | | | |
| 230 | 1 | | 2 | 3 | 11 | 245.5 | 392 | 882 | 1549 | 2086 |
| 233 | 6 | 8 | 17 | 22 | 23 | 2000 | 2489 | 3528 | 3689 | 2481 |
| 237 | 1 | | 5 | 9 | 15 | 0 | 0 | 2 | 284 | 421 |
| 238 | 1 | | 2 | 4 | 21 | 0 | 149 | 128 | 825 | 2066 |
| 242 | 1 | | 4 | 5 | 18 | 0 | 0 | 35 | 577 | 595 |
| 243 | 1 | | 2 | 5 | 23 | 0 | 0 | 0 | 456 | 615 |
| 244 | 1 | | 2 | 7 | 17 | 0 | 178 | 190 | 708 | 1112 |
| 245 | 1 | | 3 | 9 | 16 | 0 | 0 | 0 | 368 | 536 |
| 247 | 1 | | 3 | 11 | 24 | 0 | 0 | 49 | 492 | 699 |
| 248 | | | | | | 0 | 50 | 22 | 174 | 1919 |
| 249 | 2 | | 5 | 11 | 23 | 0 | 0 | 100 | 907 | 1076 |
| 251 | | | | | | 0 | 0 | 0 | 0 | 0 |
| 252 | | | | | | 0 | 0 | 0 | 0 | 0 |
| 253 | | | | | | 0 | 0 | 0 | 0 | 0 |
| 254 | 1 | 3 | 7 | 14 | 22 | 118 | 896 | 1774 | 3042 | 3035 |
| 286 | 1 | 4 | 11 | 20 | 22 | 481 | 1351 | 2882 | 3383 | 2479 |
| 287 | 1 | 1 | 3 | 11 | 23 | 97 | 398 | 986 | 2828 | 3410 |
| 315 | 11 | 14.5 | 25.5 | 32 | 34 | 2110 | 2209 | 2626 | 2965 | 2635 |
| 316 | 6.5 | 10.5 | 21 | 32 | 32.5 | 1319 | 1718 | 2848 | 2918 | 2540 |
| 317 | 3 | 4 | 9 | 26 | 35 | 551 | 624 | 776 | 1367 | 1076 |
| 331 | 4.5 | 8 | 11 | 14.5 | 30.5 | 1510 | 1649 | 2027 | 2319 | 2509 |
| 338 | 1 | 5 | 23 | 20 | 29 | 660.37 | 1625.38 | 3365.87 | 2897.62 | 2727 |
| 341 | 3 | 8 | 11 | 14 | 21 | 1325.62 | 1873 | 2039.75 | 2360.75 | 2574 |
| 343 | 1 | 1 | 2 | 5 | 29 | 262 | 281 | 450 | 570 | 1199 |
| 346 | | | | | | 235.86 | 339.82 | 620.36 | 829.32 | 1695.78 |
| 347 | 2 | 3 | 5 | 8 | 29 | 374 | 622 | 659 | 905 | 1567 |
| 349 | 1 | 8 | 11 | 16 | 24 | 1039.5 | 1598.88 | 1983.75 | 2191.25 | 2576.38 |
| 351 | 3 | 9 | 13 | 15 | 24 | 1350.67 | 1710.67 | 2030.92 | 2190.67 | 2668.54 |
| 353 | 1 | 2 | 5 | 7 | 30 | 390 | 490 | 709 | 931 | 1483 |
| 355 | 1 | 4 | 11 | 13 | 30 | 191 | 688 | 1122 | 1223 | 1519 |
| 357 | 2 | 7 | 11 | 15 | 23 | 539 | 777 | 1080 | 1362 | 1177 |
| 358 | 1 | 2 | 3 | 6 | 24 | 252 | 321 | 434 | 609 | 1192 |
| 359 | 3 | 9 | 11 | 13 | 23 | 1163.29 | 1508.79 | 1780.29 | 2067.67 | 2479.29 |
| 416 | | | | | | 33.74 | 39.82 | 56.57 | 86.78 | 1275.28 |
| 417 | | | | | | 0 | 0 | 101.13 | 639.04 | 2016.58 |
| 419 | | | | | | 58.28 | 97.36 | 221.65 | 1520.69 | 2187.94 |
| 432 | | | | | | 54.86 | 68.86 | 105.11 | 440.28 | 1594.4 |

### Example 15: Cell Lysis by Peptidomimetic Macrocycles

SJSA-1 cells were plated out one day in advance in clear, flat-bottom plates at a density of 7500 cells/well with 100 μL/well of growth media. Row H columns 10-12 were left empty to be treated with media alone. On the day of the assay, the media was exchanged with RPMI 1% FBS media to result in 90 μL of media per well. 10 mM stock solutions of the peptidomimetic macrocycles were prepared in 100% DMSO. The peptidomimetic macrocycles were diluted serially in 100% DMSO, and further diluted 20-fold in sterile water to prepare working stock solutions in 5% DMSO/water. The concentrations of the peptidomimetic macrocycles ranged from 500 μM to 62.5 μM.

10 μL of each compound solution was added to the 90 μL of SJSA-1 cells to yield final concentration of 50 μM to 6.25 μM in 0.5% DMSO-containing media. The negative control (non-lytic sample) was 0.5% of DMSO alone. The positive control (lytic) samples included 10 μM of Melittin and 1% Triton X-100. The cell plates were incubated for 1 h at 37° C. After incubation for 1 h, the morphology of the cells was examined by microscope. The plates were then centrifuged at 1200 rpm for 5 min at room temperature. 40 μL of the supernatant for each peptidomimetic macrocycle and control sample was transferred to clear assay plates. LDH release was measured using an LDH cytotoxicity assay kit. The results of the cell lysis assay are shown in TABLE 8:

TABLE 8

| | 6.25 μM | 12.5 μM | 25 μM | 50 μM |
|---|---|---|---|---|
| | % Lysed | % Lysed | % Lysed | % Lysed |
| SP# | cells (1 h LDH) | cells (1 h LDH) | cells (1 h LDH) | cells (1 h LDH) |
| 3 | 1 | 0 | 1 | 3 |
| 4 | −2 | 1 | 1 | 2 |
| 6 | 1 | 1 | 1 | 1 |
| 7 | 0 | 0 | 0 | 0 |
| 8 | −1 | 0 | 1 | 1 |
| 9 | −3 | 0 | 0 | 2 |
| 11 | −2 | 1 | 2 | 3 |
| 15 | 1 | 2 | 2 | 5 |
| 18 | 0 | 1 | 2 | 4 |
| 19 | 2 | 2 | 3 | 21 |
| 22 | 0 | −1 | 0 | 0 |
| 26 | 2 | 5 | −1 | 0 |
| 32 | 0 | 0 | 2 | 0 |
| 39 | 0 | −1 | 0 | 3 |
| 43 | 0 | 0 | −1 | −1 |
| 55 | 1 | 5 | 9 | 13 |
| 65 | 0 | 0 | 0 | 2 |
| 69 | 1 | 0.5 | −0.5 | 5 |
| 71 | 0 | 0 | 0 | 0 |
| 72 | 2 | 1 | 0 | 3 |
| 75 | −1 | 3 | 1 | 1 |
| 77 | −2 | −2 | 1 | −1 |
| 80 | 0 | 1 | 1 | 5 |
| 81 | 1 | 1 | 0 | 0 |
| 82 | 0 | 0 | 0 | 1 |
| 99 | 1.5 | 3 | 2 | 3.5 |
| 108 | 0 | 0 | 0 | 1 |
| 114 | 3 | −1 | 4 | 9 |
| 115 | 0 | 1 | −1 | 6 |
| 118 | 4 | 2 | 2 | 4 |
| 120 | 0 | −1 | 0 | 6 |
| 121 | 1 | 0 | 1 | 7 |
| 122 | 1 | 3 | 0 | 6 |
| 123 | −2 | 2 | 5 | 3 |
| 125 | 0 | 1 | 0 | 2 |
| 126 | 1 | 2 | 1 | 1 |
| 130 | 1 | 3 | 0 | −1 |
| 139 | −2 | −3 | −1 | −1 |
| 142 | 1 | 0 | 1 | 3 |
| 144 | 1 | 2 | −1 | 2 |
| 147 | 8 | 9 | 16 | 55 |
| 148 | 0 | 1 | −1 | 0 |
| 149 | 6 | 7 | 7 | 21 |
| 150 | −1 | −2 | 0 | 2 |
| 153 | 4 | 3 | 2 | 3 |
| 154 | −1 | −1.5 | −1 | −1 |
| 158 | 0 | −6 | −2 | |
| 160 | −1 | 0 | −1 | 1 |
| 161 | 1 | 1 | −1 | 0 |
| 169 | 2 | 3 | 3 | 7 |
| 170 | 2 | 2 | 1 | −1 |
| 174 | 5 | 3 | 2 | 5 |
| 175 | 3 | 2 | 1 | 0 |
| 177 | −1 | −1 | 0 | 1 |
| 182 | 0 | 2 | 3 | 6 |
| 183 | 2 | 1 | 0 | 3 |
| 190 | −1 | −1 | 0 | 1 |
| 196 | 0 | −2 | 0 | 3 |
| 197 | 1 | −4 | −1 | −2 |
| 203 | 0 | −1 | 2 | 2 |
| 204 | 4 | 3 | 2 | 0 |
| 211 | 5 | 4 | 3 | 1 |
| 217 | 2 | 1 | 1 | 2 |
| 218 | 0 | −3 | −4 | 1 |
| 219 | 0 | 0 | −1 | 2 |
| 221 | 3 | 3 | 3 | 11 |
| 223 | −2 | −2 | −4 | −1 |
| 230 | 0.5 | −0.5 | 0 | 3 |
| 232 | 6 | 6 | 5 | 5 |
| 233 | 2.5 | 4.5 | 3.5 | 6 |
| 237 | 0 | 3 | 7 | 55 |
| 243 | 4 | 23 | 39 | 64 |
| 244 | 0 | 1 | 0 | 4 |
| 245 | 1 | 14 | 11 | 56 |
| 247 | 0 | 0 | 0 | 4 |
| 249 | 0 | 0 | 0 | 0 |
| 254 | 11 | 34 | 60 | 75 |
| 279 | 6 | 4 | 5 | 6 |
| 280 | 5 | 4 | 6 | 18 |
| 284 | 5 | 4 | 5 | 6 |
| 286 | 0 | 0 | 0 | 0 |
| 287 | 0 | 6 | 11 | 56 |
| 316 | 0 | 1 | 0 | 1 |
| 317 | 0 | 1 | 0 | 0 |
| 331 | 0 | 0 | 0 | 0 |
| 335 | 0 | 0 | 0 | 1 |
| 336 | 0 | 0 | 0 | 0 |
| 338 | 0 | 0 | 0 | 1 |
| 340 | 0 | 2 | 0 | 0 |
| 341 | 0 | 0 | 0 | 0 |
| 343 | 0 | 1 | 0 | 0 |
| 347 | 0 | 0 | 0 | 0 |
| 349 | 0 | 0 | 0 | 0 |
| 351 | 0 | 0 | 0 | 0 |
| 353 | 0 | 0 | 0 | 0 |
| 355 | 0 | 0 | 0 | 0 |
| 357 | 0 | 0 | 0 | 0 |
| 359 | 0 | 0 | 0 | 0 |
| 413 | 5 | 3 | 3 | 3 |
| 414 | 3 | 3 | 2 | 2 |
| 415 | 4 | 4 | 2 | 2 |

### Example 16: p53 GRIP Assay

The p53 GRIP assay monitors the protein interaction of p53 and MDM2, and the cellular translocation of GFP-tagged p53 in response to drug compounds or other stimuli. Recombinant CHO-hIR cells stably express human p53 (1-312) fused to the C-terminus of enhanced green fluorescent protein (EGFP) and PDE4A4-MDM2 (1-124), a fusion protein between PDE4A4 and MDM2 (1-124). The effects of experimental conditions on the interaction of p53 and MDM2 can be measured.

CHO-hIR cells were regularly maintained in Ham's F12 media supplemented with penicillin-streptomycin, 0.5 mg/ml Geneticin, 1 mg/ml Zeocin, and 10% FBS. Cells were seeded into 96-well plates at a density of 7000 cells/100 μL/well using culture media 18-24 h prior to running the assay. On the day of the assay, the media was refreshed, and PD-177 was added to cells to reach a final concentration of 3 μM to activate foci formation. Control wells were kept without PD-177. 24 h post-stimulation with PD-177, the cells were washed once with reduced-serum media. 50 μL of the reduced-serum media supplemented with PD-177 (6 μM) was added to the cells. The peptides were diluted from 10 mM DMSO stock solutions to 500 μM working stock solutions in sterile water. Further dilutions were made in 0.5% DMSO to keep the concentration of DMSO constant across the samples. The final highest DMSO concentration was 0.5%, and 0.5% DMSO was used as the negative control. (−)-Nutlin-3 (10 mM) was used as a positive control. Nutlin was diluted using the same dilution scheme used for the peptides.

50 μL of the 2× desired concentration peptide solutions were added to the appropriate wells to achieve desired final concentrations. Cells were then incubated with the peptides for 6 h at 37° C. in a humidified 5% CO₂ atmosphere. After incubation, the cells were fixed by gently aspirating out the media and adding 150 μL of fixing solution per well for 20 minutes at room temperature. The fixed cells were washed 4 times with 200 μL PBS per well each time. At the end of last wash, 100 L of 1 M Hoechst staining solution was added. The sealed plates were incubated for at least 30 min in the dark, and washed with PBS to remove excess staining solution. PBS was added to each well. The plates could be stored at 4° C. in the dark for up to 3 days. The translocation of p53/MDM2 was imaged using a molecular translocation module using 10× objective and XF-100 filter sets for Hoechst and GFP. The minimal acceptable number of cells per well used for image analysis was set to 500 cells.

### Example 17: MCF-7 Breast Cancer Study Using SP315, SP249 and SP154

A xenograft study was performed to test the efficacy of SP315, SP249 and SP154 in inhibiting tumor growth in athymic mice in the MCF-7 breast cancer xenograft model. A negative control stapled peptide (SP252) and a point mutation of SP154 (F to A at position 19) were tested in one group. The negative control stapled peptide exhibited no activity in the SJSA-1 in vitro viability assay. Slow release 90 day 0.72 mg 17β-estradiol pellets were implanted subcutaneously (sc) on the nape of the neck one day prior to tumor cell implantation (Day −1). On Day 0, MCF-7 tumor cells were implanted sc in the flank of female nude (Cr1:NU-Foxn1nu) mice. On Day 18, the resultant sc tumors were measured using calipers to determine their length and width, and the mice were weighed. The tumor sizes were calculated using the formula (length×width²)/2, and expressed as cubic millimeters (mm³). Mice with tumors smaller than 85.3 mm³ or larger than 417.4 mm³ were excluded from the subsequent group formation. Thirteen groups of mice, 10 mice per group, were formed by randomization such that the group mean tumor sizes were essentially equivalent (mean of groups±standard deviation of groups=180.7±17.5 mm³).

SP315, SP249, SP154 and SP252 dosing solutions were prepared from peptides formulated in a vehicle containing MPEG(2K)-DSPE at 50 mg/mL concentration in a 10 mM histidine-buffered saline solution at pH 7. The peptide formulations were prepared once for the duration of the study. The vehicle was used as the vehicle control in the subsequent study.

Each group was assigned to a different treatment regimen. Group 1, as the vehicle negative control group, received the vehicle administered at 8 mL/kg body weight intravenously (I.V.) three times per week from Days 18-39. Group 2 received SP154 as an I.V. injection at 30 mg/kg three times per week. Group 3 received SP154 as an I.V. injection at 40 mg/kg twice a week. Group 4 received 6.7 mg/kg SP249 as an I.V. injection three times per week. Group 5 received SP315 as an I.V. injection of 26.7 mg/kg three times per week. Group 6 received SP315 as an I.V. injection of 20 mg/kg twice per week. Group 7 received SP315 as an I.V. injection of 30 mg/kg twice per week. Group 8 received SP315 as an I.V. injection of 40 mg/kg twice per week. Group 9 received 30 mg/kg SP252 as an I.V. injection three times per week.

During the dosing period, the mice were weighed and the tumors were measured 1-2 times per week. Tumor growth inhibition was compared with the vehicle group. Changes in body weight and number of mice with ≥20% body weight loss or death is shown in TABLE 9. Tumor growth inhibition (TGI) was calculated as

% TGI=100−[(TuVol^{Treated-day x}−TuVol^{Treated-day 18})/(TuVol^{Vehicle negative control-day x}−TuVol^{vehicle negative control-day 18})*100,

where x=day that effect of treatment is being assessed. Group 1, the vehicle negative control group, showed good tumor growth rates.

2 mice died during treatment with SP154 when dosed with 40 mg/kg twice a week. The dosing regimen of 30 mg/kg of SP154 three times per week yielded a TGI of 84%. 4 mice died in the group dosed with SP249 6.7 mg/kg three times. No body weight loss or deaths were noted for all groups treated with SP315. Dosing with 40 mg/kg of SP315 twice per week produced the highest TGI (92%). The dosing regimens of SP315 of 26.7 mg/kg three times per week, 20 mg/kg twice per week, 30 mg/kg twice per week produced TGI of 86, 82, and 85%, respectively. No body weight loss or deaths were noted for the group treated with SP252 30 mg/kg three times per week. The TGI was 88% on day 32, and reduced to 41% by day 39.

TABLE 9

| Group | | % BW | No. with ≥10% | No. with ≥20% | |
|---|---|---|---|---|---|
| Number | Treatment Group | Change | BW Loss | BW Loss or death | % TGI |
| 1 | Vehicle | +8.6 | 0/10 | 0/10 | — |
| 2 | SP154 30 mg/kg | +5.7 | 0/10 | 0/10 | *84 |
| | 3x/wk I.V. | | | | |
| 3 | SP154 40 mg/kg | N/A | 0/10 | 2/10 (2 deaths) | Regimen not |
| | 2x/wk I.V. | | | | tolerated |
| 4 | SP249 6.7 mg/kg | N/A | 6/10 | 4/10 | Regimen not |
| | 3x/wk I.V. | | | | tolerated |
| 5 | SP315 26.7 mg/kg | +3.7 | 0/10 | 0/10 | *86 |
| | 3x/wk I.V. | | | | |
| 6 | SP315 20 mg/kg | +3.9 | 0/10 | 0/10 | *82 |
| | 2x/wk I.V. | | | | |
| 7 | SP315 30 mg/kg | +8.0 | 0/10 | 0/10 | *85 |
| | 2x/wk I.V. | | | | |
| 8 | SP315 40 mg/kg | +2.1 | 0/10 | 0/10 | *92 |
| | 2x/wk I.V. | | | | |
| 9 | SP252 30 mg/kg | +3.3 | 0/10 | 0/10 | *41 |
| | 3x/wk I.V. | | | | |

*p ≤ 0.05 Vs Vehicle Control

### Example 18: Testing of Peptidomimetic Macrocycles for Ability to Reduce Immune Checkpoint Protein Expression or Inhibit Immune Checkpoint Protein Activity

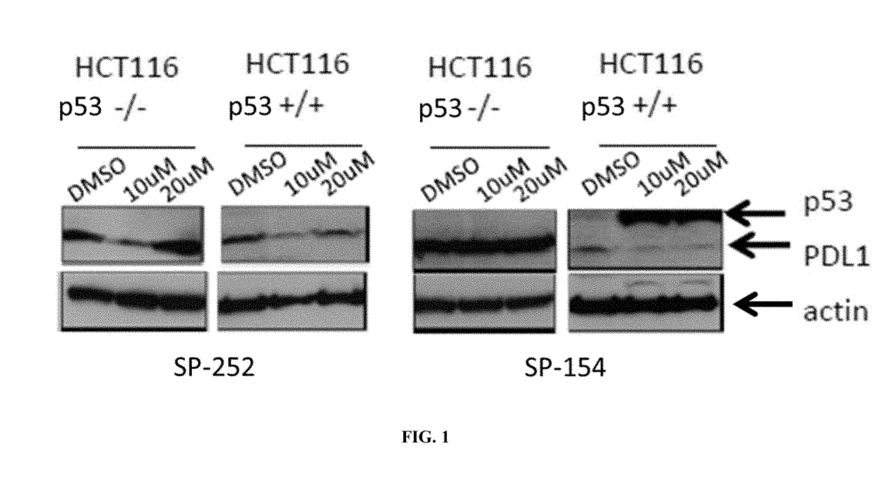

Assays were performed to determine whether the peptidomimetic macrocycles can diminish PD-L1 activity or expression. HCT-116 p53^{+/+} cells and HCT-116 p53^{-/-} cells were treated with DMSO or 10 μM SP or 20 μM SP. FIG. 1 shows that treatment with SP262 and SP154 resulted in decreased PD-L1 expression in HCT-116 p53^{+/+} cells, but not HCT-116 p53^{-/-} cells. Similar assays are performed in cell lines that express higher levels of PD-L1, such as A549 cells, H460 cells, and syngeneic mouse cell lines.

Assays are performed to determine whether the peptidomimetic macrocycles can diminish PD-L1 activity or expression via miR-34a to enhance immune response against tumors. Assays are performed to determine whether the peptidomimetic macrocycles of the invention mimic the immune-enhancing effects of anti-PD-1 and/or anti-PD-L1 agents, with the added benefit of inducing cell cycle arrest and apoptosis.

Cancer cells from different lineages MCF-7 (breast), HCT-116 (large intestine), MV4-11 (leukemia), DOHH2, and A375 (melanoma) are dosed with peptidomimetic macrocycles. These cell lines and others are selected to include cell lines that have high levels of PD-L1 expression and others that have low levels of PD-L1 expression. Changes in protein and mRNA levels of PD-1, PD-L1 and miR-34a are measured, for example, using flow cytometry. p53 and p21 are used as controls. RT-PCR assays are performed to quantify miR-34a, miR-34b, and/or miR-34c levels in samples in parallel with flow cytometry measurements. Full dose-response curves are taken 24, 48, and 72 hours after dosing. Additionally, apoptosis measurements are made in parallel.

### Example 19: Phase I Dose-Escalation Clinical Trial for AP1

A dose-escalation study was conducted in a Phase 1 open-label, multi-center, two-arm trial of the compound AP1. AP1 was administered by I.V. infusion to patients with advanced solid tumors or lymphomas expressing WT p53 that was refractory to or intolerant of standard therapy or for which no standard therapy existed. The trial established a 3.1 mg/kg dose of AP1 as the MTD (i.e., the highest dose of the drug that did not cause unacceptable side effects) when dosed once a week by I.V. administration. The trial also evaluated the safety, tolerability, and the pharmacokinetics of AP1 and provided a preliminary assessment of activity using pharmacodynamic biomarkers and imaging assessments.

71 patients were enrolled in the dose-escalation trial. The trial used a “3+3” dose-escalation design. Patients in the first two dose levels received AP1 once a week for three consecutive weeks over a 28-day cycle or a lower dose level twice a week for two consecutive weeks over a 21-day cycle. The dose-escalation study was used to evaluate different benefit-risk ratios and provide supporting evidence for dose selection during the clinical development of AP1.

Starting with dose level 4, patients who had cancers associated with HPV were excluded from enrollment because HPV is known to destroy WT p53. Dosing started at relatively low dose levels, and the protocol did not require patients in the first three dose levels to have WT p53 or cancers unassociated with HPV because the trial focused primarily on the safety and tolerability of AP1.

To identify specific WT p53 patients for the clinical trials, commercially available third-party assays and a central laboratory were used to conduct next generation sequencing on archived tumor tissue samples or fresh biopsy samples from patients taken prior to enrollment.

WT p53 status was not required of the patients for the initial three dose levels prior to enrollment. The patients' WT p53 statuses were established through testing after enrollment. Seven of the 13 patients enrolled in those dose levels who completed at least one cycle were confirmed to have WT p53 status, the status of four was unknown, and two patients tested positive for mutated p53. Starting with dose level 4, WT p53 status was a mandatory eligibility criterion.

Clinical activity or a patient's response to AP1 was determined using pharmacodynamics (PD) biomarkers and imaging assessments. PD biomarkers provided information on on-target activity, specific patient type responses, and provided an early insight on AP1's effect on tumors. The effect of AP1 on potential PD biomarkers was determined for different sources of biological samples, such as tumor biopsies, circulating tumor cells where detectable, mononuclear blood cells, and blood and bone marrow samples. Depending on the sample type, the PD biomarkers included measures of MDMX, MDM2, p21, p53, apoptosis, macrophage inhibitory cytokine 1, or MIC-1. Standard imaging assessments, such as computed tomography (CT) or positron emission tomography (PET), were used to obtain images from patients.

Anti-tumor activity was measured using Response Evaluation Criteria in Solid Tumors (RECIST) criteria for patients with solid tumors, and 2015 International Working Group (IWG) criteria for patients with lymphomas. The RECIST and IWG criteria enabled the objective evaluation of whether a tumor had progressed, stabilized, or decreased in size. Anti-tumor effects were also determined through physical examinations or clinically validated blood or serum tumor markers.

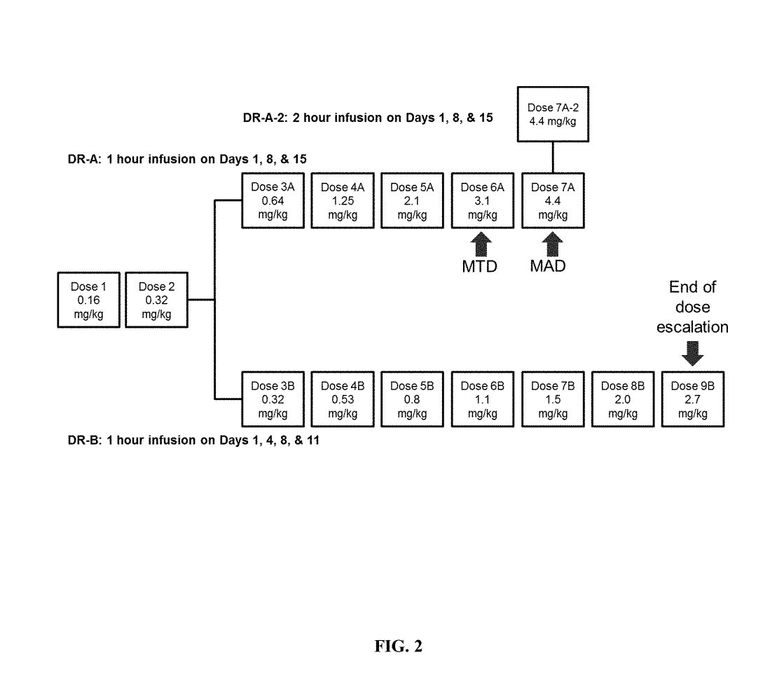

FIG. 2 illustrates the dosing regiments (DRs) used in the “3+3” dose escalation trial. DR-A depicts patients that received AP1 once a week for three consecutive weeks over a 28-day cycle. DR-B depicts patients who received lower doses of AP1 twice a week for two consecutive weeks over a 21-day cycle. The MTD of AP1 was 3.1 mg/kg, and the multiple-ascending dose (MAD) ended at 4.4 mg/kg.

### Example 20: Pharmacokinetic Profile of AP1

AP1 was delivered systematically using I.V. administration because of the potential advantage of avoiding metabolic impact from hepatic and gastrointestinal enzymes as well as reproducible systemic bioavailability.

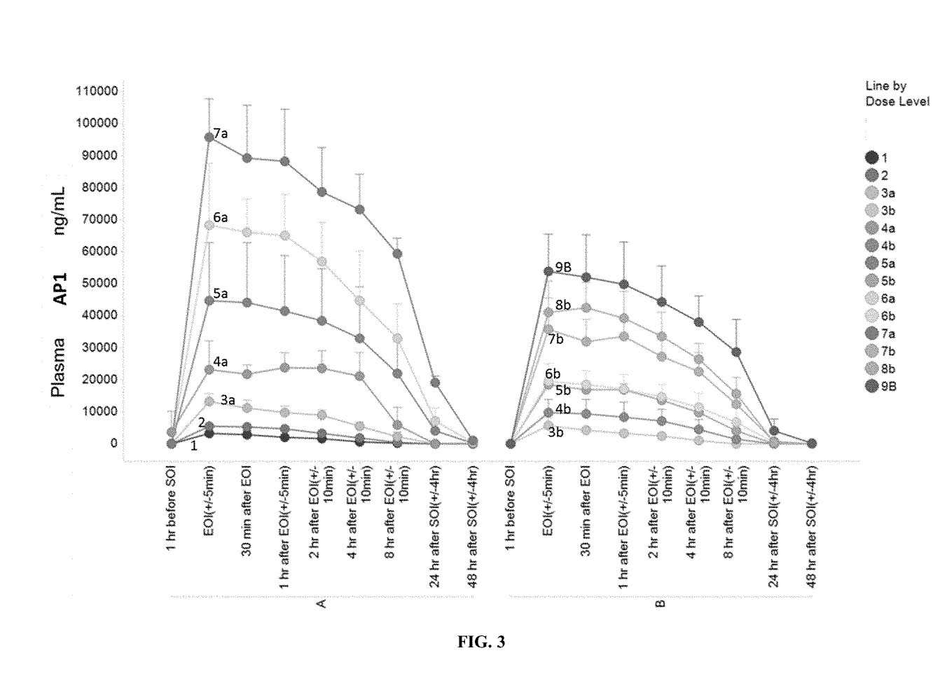

FIG. 3 shows drug concentration levels in patient plasma at all dose levels tested in Arm A (left panel) and Arm B (right panel). AP1 consistently produced a dose-related increase in maximum drug serum concentrations observed (Cₘₐₓ) in patients, and longer corresponding half-lives of between five and seven hours at higher dose levels. Data were collected at different time points after the start of infusion (SOI) and the end of infusion (EOI).

### Example 21: Safety Results for AP1

AP1 has been considered by the dose escalation trial's investigators to be generally well tolerated. The most frequently reported drug-related events to date reported by ≥10% of the patients were nausea, fatigue, vomiting, decreased appetite, anemia, headache, and constipation. Transient decreases in lymphocytes post-dosing and primarily Grade 1 and 2 abnormalities were observed in approximately 50% of patients with full recovery within a few days.

Seven patients experienced infusion-related reactions, and administration of AP1 was discontinued for three patients. Eight patients experienced dose reductions, including two patients who had been on study treatment for over 1 year, and another patient who had been on study treatment for 11 months. One dose limiting toxicity (DLT) of Grade 3 fatigue was reported at 3.1 mg/kg once weekly dosing, and four DLTs (Grade 3 elevated alkaline phosphatase, Grade 3 hypotension, Grade 3 anemia, Grade 4 neutropenia) were reported at 4.4 mg/kg once weekly. Nine severe adverse events (SAEs) were reported, two of which were related to AP1. Both events were Grade 3 hypotension and were at the 3.1 mg/kg and the 4.4 mg/kg once-weekly dosing levels. Grade 3/4 events that were at least possibly related to AP1 occurred in fifteen patients, and included anemia (n=2), an increase in blood alkaline phosphatase levels, diarrhea, fatigue (n=3), hyponatremia, hypotension (n=2), hypoxia, nausea, neutropenia (n=3), and vomiting. Five patients discontinued treatment with AP1 due to these events.

### Example 22: Biomarker Assessments for the Biological Activity of AP1

Several exploratory biomarkers were used to confirm the pharmacological or on-target biological activity of AP1, aid patient recruitment, and help inform dose selection. In the Phase 1 dose-escalation study, plasma MIC-1 levels were measured at several time points after initial infusion.

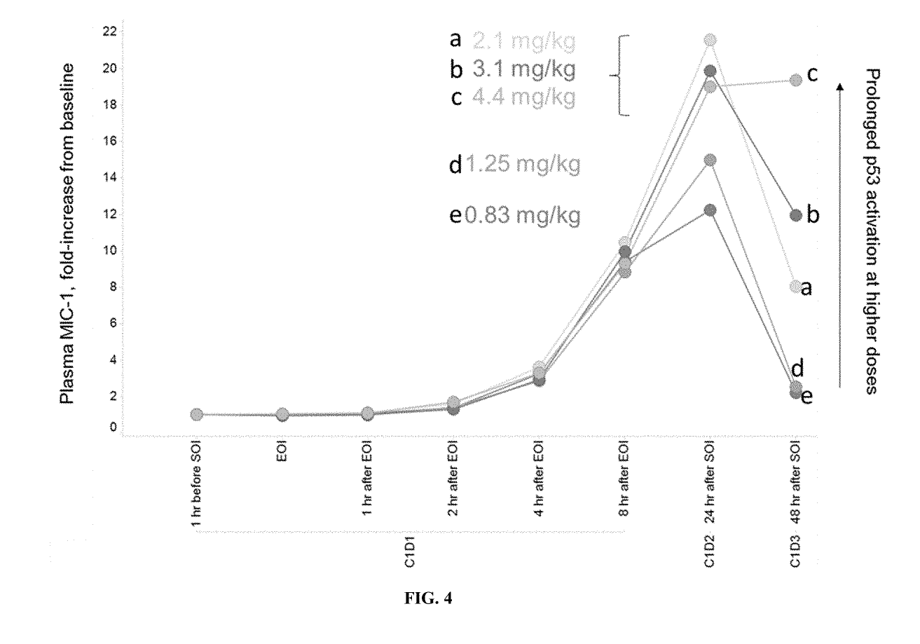

FIG. 4 shows fold-increase levels from baseline levels of plasma MIC-1 on cycle one, day one, two, or three (C1D1, C1D2, C1D3) at dose levels at or above 0.83 mg/kg. The results demonstrated that dose-related, on-mechanism increased in MIC-1 blood levels after the end of AP1 infusion (EOI) achieved a maximal 24 hr MIC-1 increase above baseline at doses ≥2.1 mg/kg. Prolonged p53 activation of MIC-1 was observed 48 hours after the start of infusion (SOI).

### Example 23: Clinical Activity of AP1

Clinical activity or responses to AP1 were assessed using imaging methods. Anti-tumor activity was measured using RECIST criteria for patients with solid tumors and the IWG criteria for patients with lymphomas to objectively evaluate whether a tumor progressed, stabilized, or shrunk. In the dose-escalation Phase 1 trial patients in Arm A (28-day cycle group) of the pharmacokinetic study (EXAMPLE 20), plasma AP1 levels were measured at baseline and again after two cycles of study medication, or approximately within 56 days following initial dosing and every 2 cycles thereafter. Patients in Arm B (21-day cycle group of the pharmacokinetic study (EXAMPLE 20), plasmas AP1 levels were measured at baseline and again after three cycles of study medication, or approximately within 63 days following initial dosing and every three cycles thereafter.

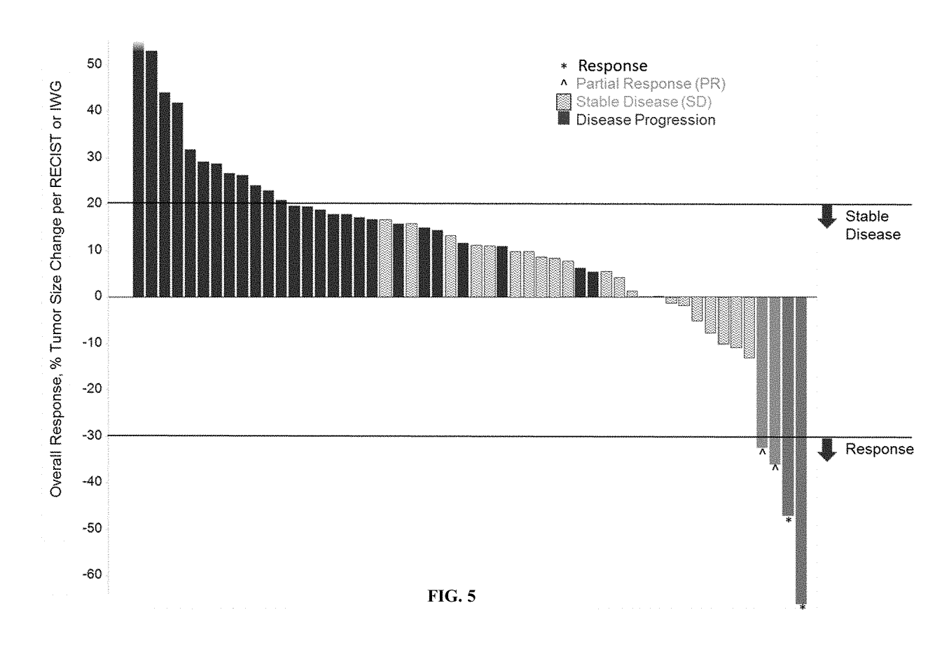

FIG. 5 shows a waterfall plot that illustrates the anti-tumor activity of AP1 in patients of the Phase 1 dose-escalation trial. The percent change in tumor volume for each evaluable patient (i.e., having measurable disease by CT or PET-CT scan) is plotted from the highest to lowest value, or low to high response, and each bar of the histogram is colored by the best overall response measured for that patient per RECIST or IWG criteria.

57 patients were evaluated using RECIST or IWG guidelines, including the 52 with CT- or PET-CT scans shown in FIG. 5, and five with clinical or objective evidence of disease progression without scans. Of the evaluable patients, 25 (44%) patients demonstrated disease control in at least one scan following the start of AP1 therapy. There were two responses (CRs), two partial responses (PRs), and 21 responses with stabilization of tumor size (SDs). The latter included 7 stable diseased patients that exhibited tumor shrinkage.

The anti-tumor activity of the Phase 1 dose-escalation trial compared favorably to results of Phase 1 trials using valuable oncology agents, such as nivolumab and pembrolizumab. The results for AP1 in 57 patients included 2 R₅, 2 PRs, and 21 (7 shrinkages). For 39 patients treated with nivolumab, the results were 1 R, 2 PRs, and 12 SDs (2 shrinkages). For 30 patients treated with pembrolizumab, the results were 2 R₅, 3 PRs, and 11 SDs (3 shrinkages).

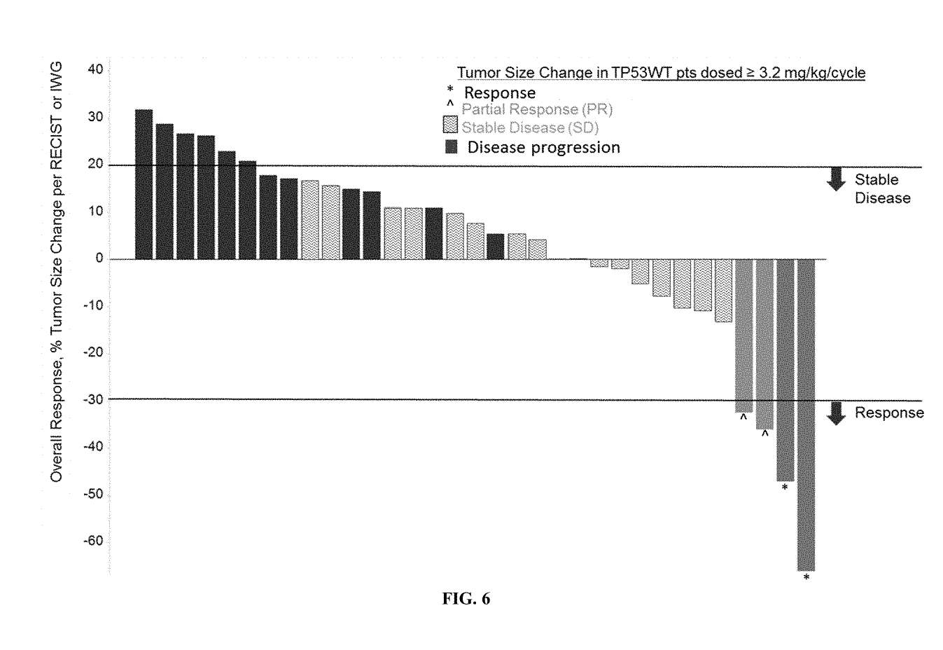

AP1 yielded a disease control rate of 20/35 (56%) when considering the anti-tumor activity of the Phase 1 dose-escalation trial at doses most relevant to continued clinical development (≥3.2 mg/kg/cycle) and limiting analyses to patients with WT p53. FIG. 6 shows results of the anti-tumor activity study for 33 patients. The study also included results for three additional patients with clinical or objective evidence of disease progression without imaging scans.

The duration of time a patient continued treatment with AP1 served as an additional measure of anti-tumor activity and continued response to AP1 therapy. FIG. 7 shows the time-on-drug for evaluable p53-WT patients who had CRs, PRs, and SDs when dosed with AP1 at ≥3.2 mg/kg/cycle. The median time a patient received AP1 was 147 days, with an average of 192 days, and a max for one patient of 613 days. Three patients received AP1 for over a year, and all patients who achieved R₅ or PRs since inception of the trial remained on AP1 therapy.

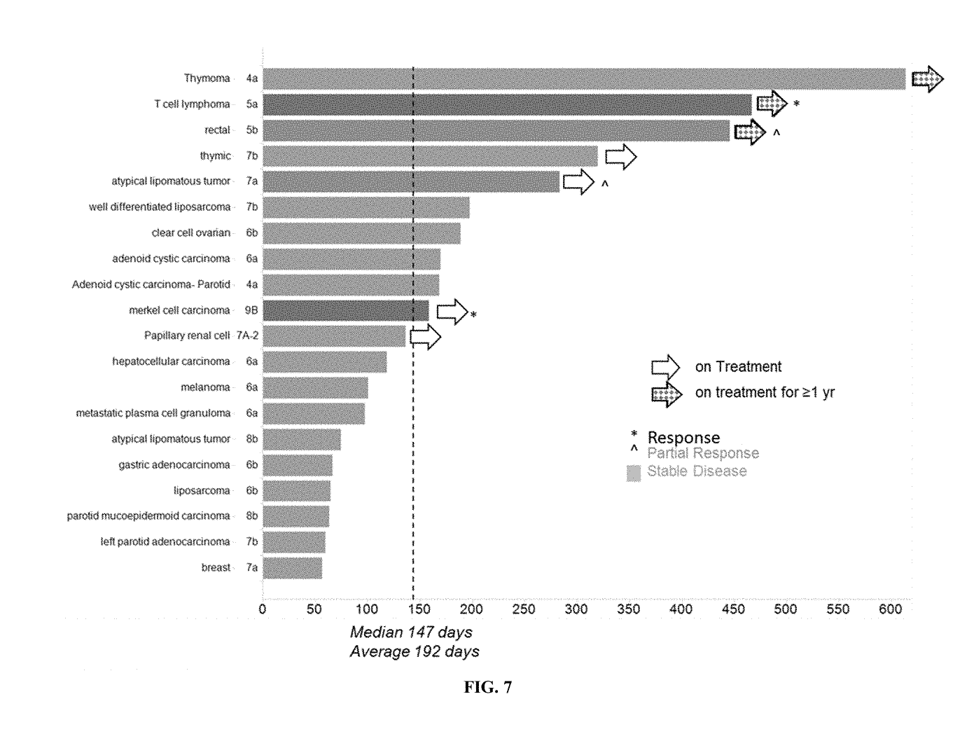

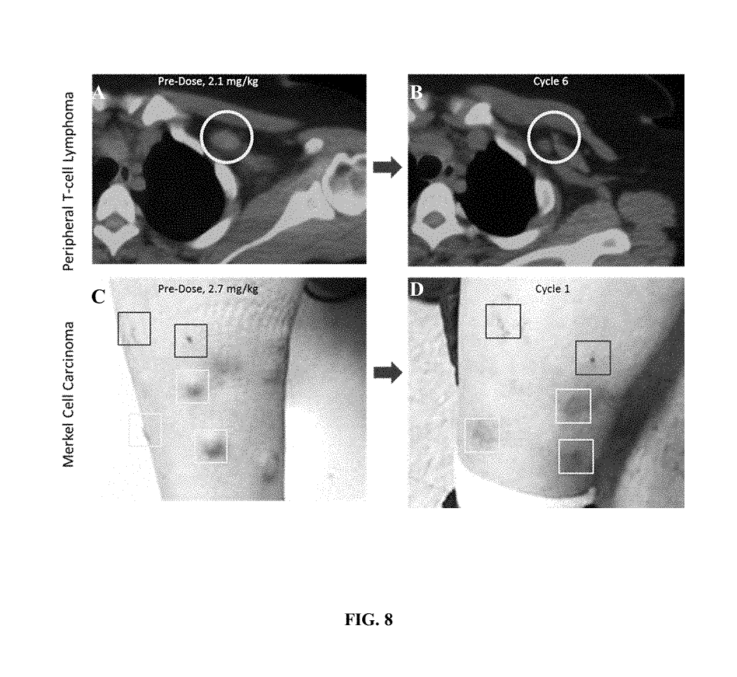

FIG. 8 PANEL A-FIG. 8 PANEL D shows patient scans from two CR patients observed in the dose-escalation Phase 1 trial. FIG. 8 PANEL A shows a 50-year-old patient with peripheral T-Cell Lymphoma (PTCL), a highly aggressive form of non-Hodgkin's lymphoma. The images showed a strong signal for aberrant cellular metabolism, which indicated cancer in a lymph node of the patient's chest. After six cycles of AP1 treatment, the lymph node returned to its normal size and no was longer detected by the PET tracer as being cancerous (FIG. 8 PANEL B).

FIG. 8 PANEL C shows images of a 73-year-old patient with Merkel Cell Carcinoma (MCC), a highly aggressive skin cancer. The patient exhibited skin lesions consistent with MCC. After one cycle of AP1 therapy, the skin lesions diminished in size and left only mild scar tissue (FIG. 8 PANEL D). After further treatment with AP1, a biopsy sample from the formerly tumorous skin areas and PET-CT scans showed no trace of cancer in the patient.

### Example 24: Phase 2a Clinical Trials with AP1 in Patients with Peripheral T-Cell Lymphoma

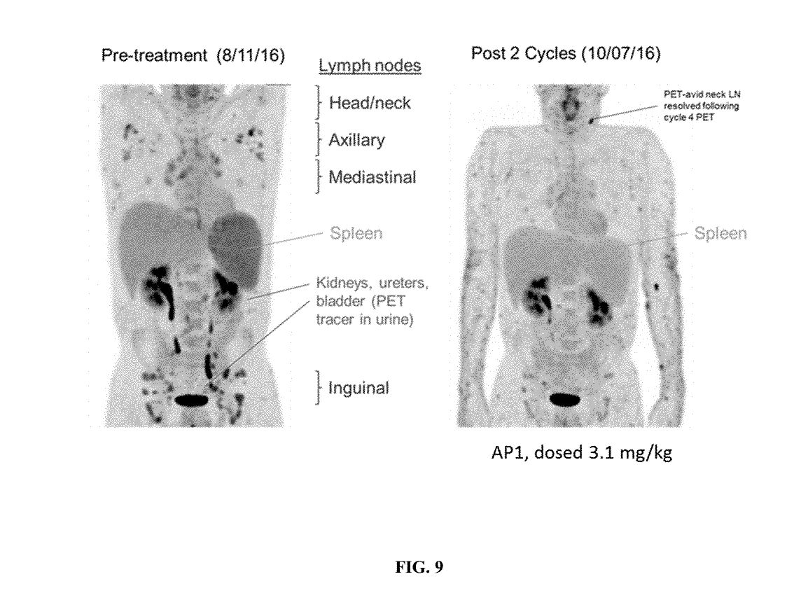

Based on the results of the dose-escalation study and the complete response observed in a patient with PTCL, a Phase 2a clinical trial was conducted in patients with PTCL. The first patient enrolled in the Phase 2 study in PTCL achieved a partial response. FIG. 9 LEFT PANEL shows PET scans from the first patient enrolled in the Phase 2 study prior to treatment with AP1. FIG. 9 RIGHT PANEL shows PET scans from the first patient enrolled in the Phase 2 study after 2 cycles of treatment with AP1. Before beginning treatment with AP1, a 39-year-old male patient exhibited strong signals for aberrant cellular metabolism indicative of cancer in the lymph nodes of his neck, under his arms, in his chest, and in his groin (FIG. 9 LEFT PANEL). Following two cycles of treatment with AP1, the lymph nodes picked up a substantially reduced amount of the PET tracer that would indicate the lymph nodes were cancerous (FIG. 9 RIGHT PANEL).

TABLE 10 shows Phase 2a study results of seven PTCL patients on AP1 therapy, with details on the status, days on AP1 treatment and best overall response of each patient.

TABLE 10

| Patient | | | Days on | Best overall |
|---|---|---|---|---|
| # | Study | Status | treatment | response |
| 1 | Dose escalation | Ongoing | 487 | CR |
| 2 | Phase 2a | Disease progression | 122 | PR |
| 3 | Phase 2a | Ongoing | 134 | SD |
| 4 | Phase 2a | Disease progression | 66 | ODP |
| 5 | Phase 2a | Ongoing | 53 | Tbd |
| 6 | Phase 2a | Ongoing | 32 | Tbd |
| 7 | Phase 2a | Ongoing | 1 | Tbd |

### Example 25: Survival in an In Vivo Xenotransplantation Model

AP1 was tested for overall survival in an in vivo xenotransplantation model. Engraftment of human CD45 leukemic cells after 5 weeks ranged from 1% to 73% in vehicle and 0% to 0.05% in AP1 treated animals. Mice treated with AP1 lived significantly longer than vehicle treated counterparts. The median survival for group 1 and group 2 was 34 days and 83 days, respectively (p<0.0001). Long term survival was assessed at 130 days post start of treatment, with 22% of mice in group 2 and 60% of mice in group 3 still alive.

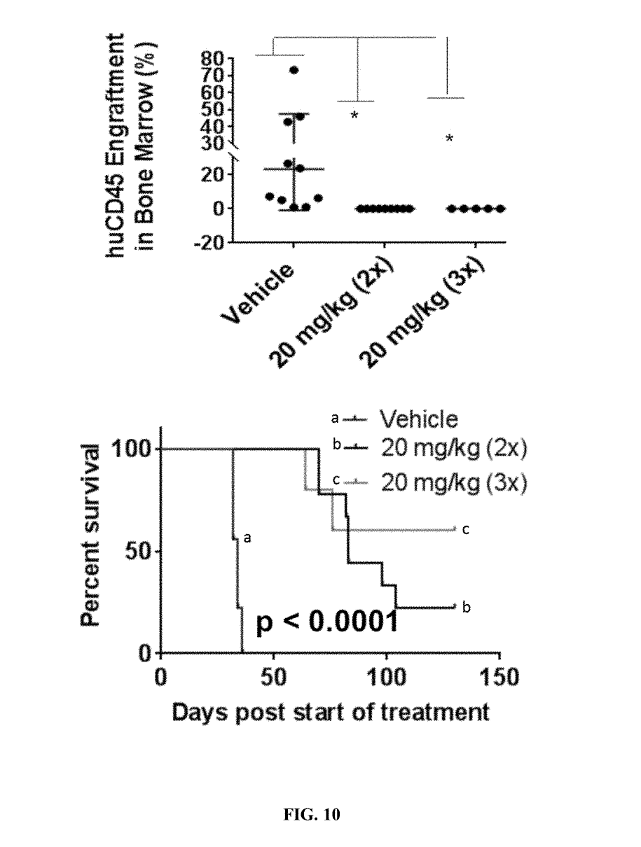

Treatment with AP1 doubled the overall survival in an in vivo implantation model. FIG. 10 TOP PANEL shows percentage of human CD45 engraftment in bone marrow for the vehicle, and treatment with 20 mg/kg AP1. FIG. 10 BOTTOM PANEL shows the percentage survival of mice upon treatment with the vehicle or administration of AP1.

### Example 26: WST-1 Cell Proliferation Assays

The WST-1 variant of the MTT assay was used to measure cell viability. WST-1 is a cell-impermeable, sulfonated tetrazolium salt that can be used to examine cell viability without killing the cells. The human tumor cell lines MCF-7 and MOLT-3 were grown in EMEM and RPMI1640, respectively. All media were supplemented with 10% (v/v) fetal calf serum, 100 units of penicillin, and 100 μg/mL of streptomycin at 37° C. and 5% CO₂. Prior to dosing, MCF-7 cells were switched to serum-free medium and grown at 37° C. overnight. One day prior to assaying, the cells were trypsinized, counted, and seeded at pre-determined densities in 96-well plates as follows: MCF-7, 5000 cells/well/200 μL; MOLT-3, 30,000 cells/well/200 μL.

FIG. 11 shows a graph of MCF-7 cell proliferation determined using a WST-1 assay measured at the indicated time points after different numbers of MCF-7 cells were grown at 37° C. for a 24 hour growth period. The MCF-7 cells were not treated with any peptides or compounds.

### Example 27: Combination Therapy with AP1 and CDK4/CDK6 Inhibitors

a. Combination Therapy with AP1 and Ribociclib

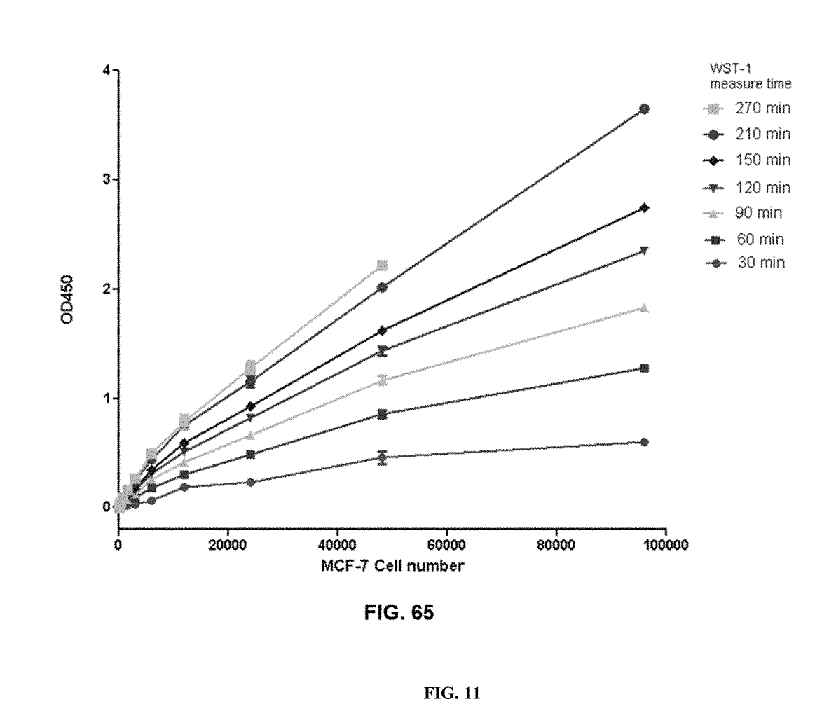

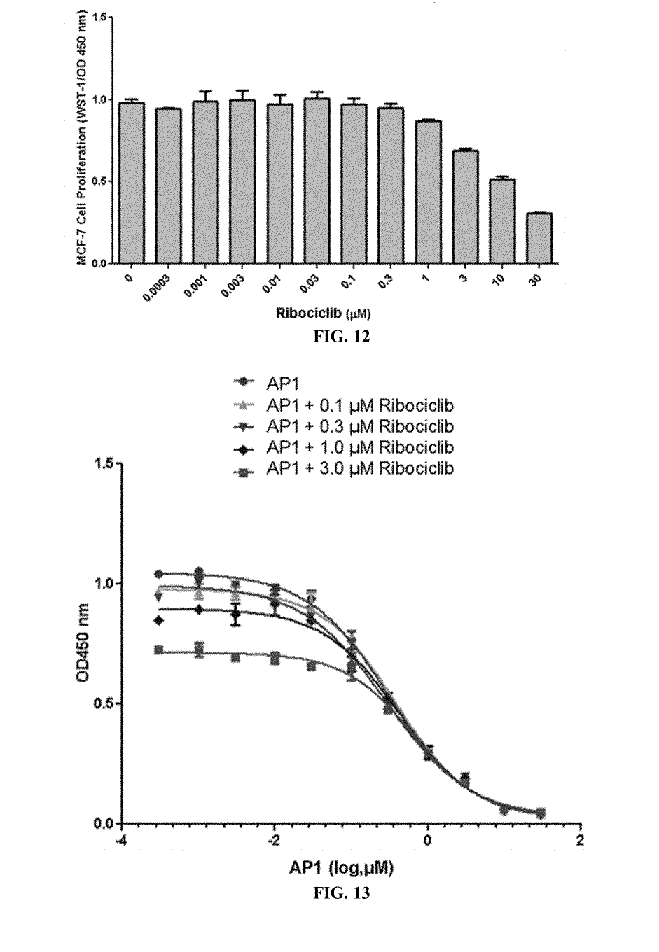

MCF-7 cell proliferation was measured using the WST-1 assay described in EXAMPLE 26. MCF-7 cells were treated with ribociclib at concentrations of 0 μM, 0.0003 μM, 0.001 μM, 0.003 μM, 0.01 μM, 0.03 μM, 0.1 μM, 0.3 μM, 1 μM, 3 μM, 10 μM, and 30 μM. FIG. 12 shows MCF-7 cell proliferation when the cells were treated with varying concentrations of ribociclib. MCF-7 cells were treated with AP1 or a combination of AP1 and ribociclib at concentrations of 0.1 μM, 0.3 μM, 1 μM, and 3 μM. The concentration of AP1 was kept constant. FIG. 13 shows MCF-7 cell proliferation when the cells were treated with AP1 or AP1 with varying concentrations of ribociclib.

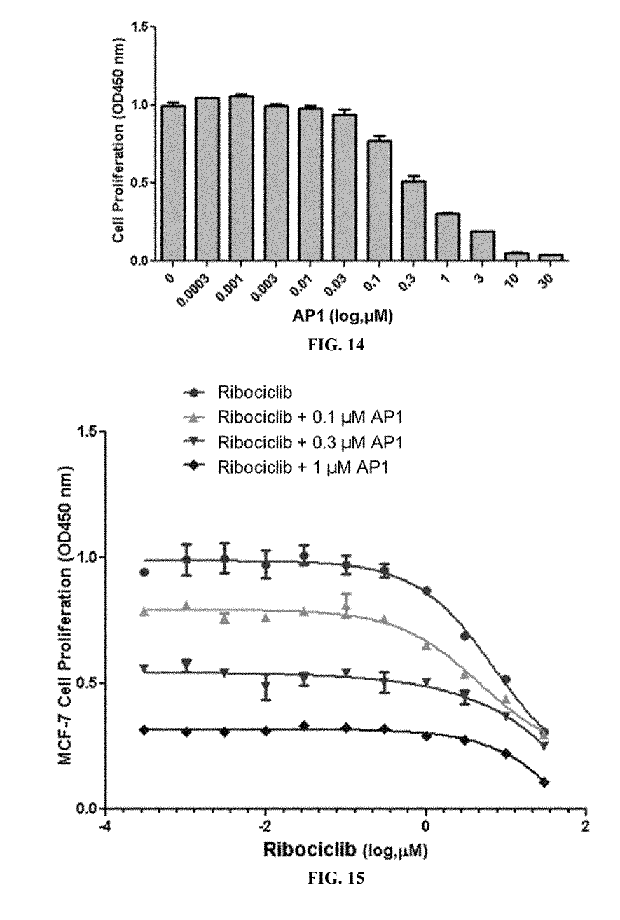

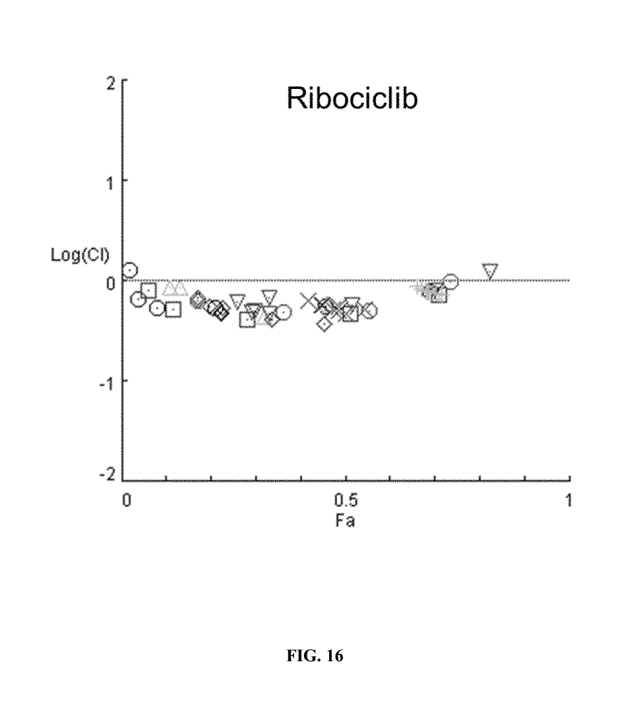

MCF-7 cells were treated with AP1 at concentrations of 0 μM, 0.0003 μM, 0.001 μM, 0.003 μM, 0.01 μM, 0.03 μM, 0.1 μM, 0.3 μM, 1 μM, 3 μM, 10 μM, and 30 μM. FIG. 14 shows MCF-7 cell proliferation when the cells were treated with varying concentrations of AP1. MCF-7 cells were treated with ribociclib or a combination of ribociclib and AP1 at concentrations of 0.1 μM, 0.3 μM, and 1 μM. The concentration of ribociclib was kept constant. FIG. 15 shows MCF-7 cell proliferation when the cells were treated with ribociclib or ribociclib with varying concentrations of AP1. FIG. 16 shows a combination index plot of ribociclib in MCF-7 cells.

b. Combination Therapy with AP1 and Abemaciclib

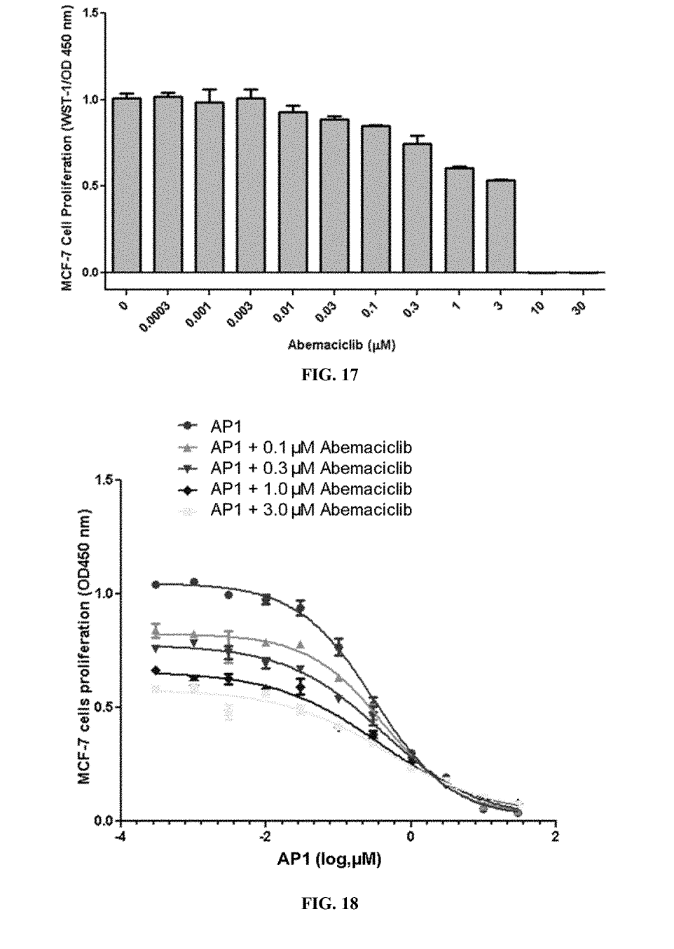

MCF-7 cell proliferation was measured using the WST-1 assay described in EXAMPLE 26. MCF-7 cells were treated with abemaciclib at concentrations of 0 μM, 0.0003 μM, 0.001 μM, 0.003 μM, 0.01 μM, 0.03 μM, 0.1 μM, 0.3 μM, 1 μM, 3 μM, 10 μM, and 30 μM. FIG. 17 shows MCF-7 cell proliferation when the cells were treated with varying concentrations of abemaciclib. MCF-7 cells were treated with AP1 or a combination of AP1 and abemaciclib at concentrations of 0.1 μM, 0.3 μM, 1 μM, and 3 μM. The concentration of AP1 was kept constant. FIG. 18 shows MCF-7 cell proliferation when the cells were treated with AP1 or AP1 with varying concentrations of abemaciclib.

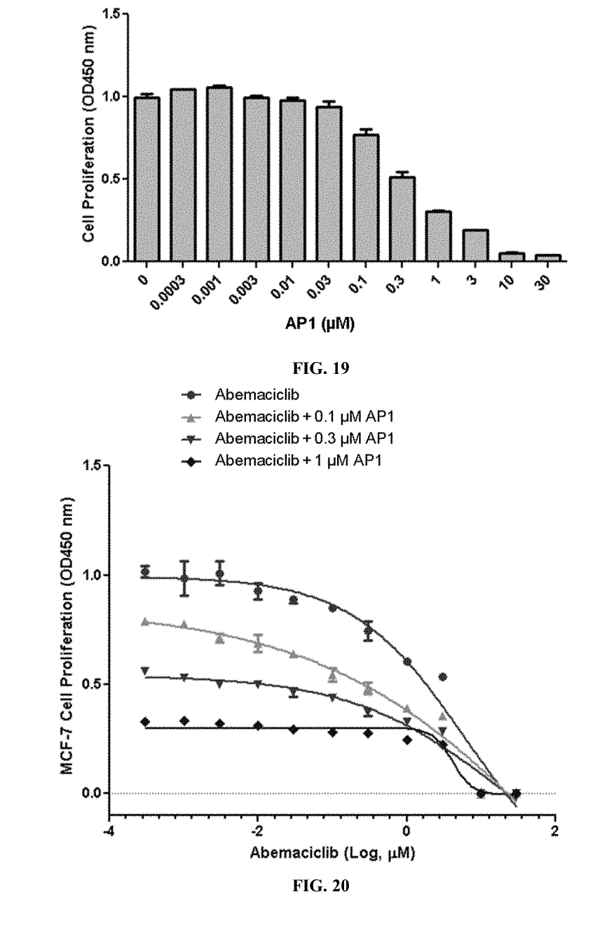

MCF-7 cells were treated with AP1 at concentrations of 0 μM, 0.0003 μM, 0.001 μM, 0.003 μM, 0.01 μM, 0.03 μM, 0.1 μM, 0.3 μM, 1 μM, 3 μM, 10 μM, and 30 μM. FIG. 19 shows MCF-7 cell proliferation when the cells were treated with varying concentrations of AP1. MCF-7 cells were treated with abemaciclib or a combination of abemaciclib and AP1 at concentrations of 0.1 μM, 0.3 μM, and 1 μM. The concentration of abemaciclib was kept constant. FIG. 20 shows MCF-7 cell proliferation when the cells were treated with abemaciclib or abemaciclib with varying concentrations of AP1.

c. Combination Therapy with AP1 and Palbociclib

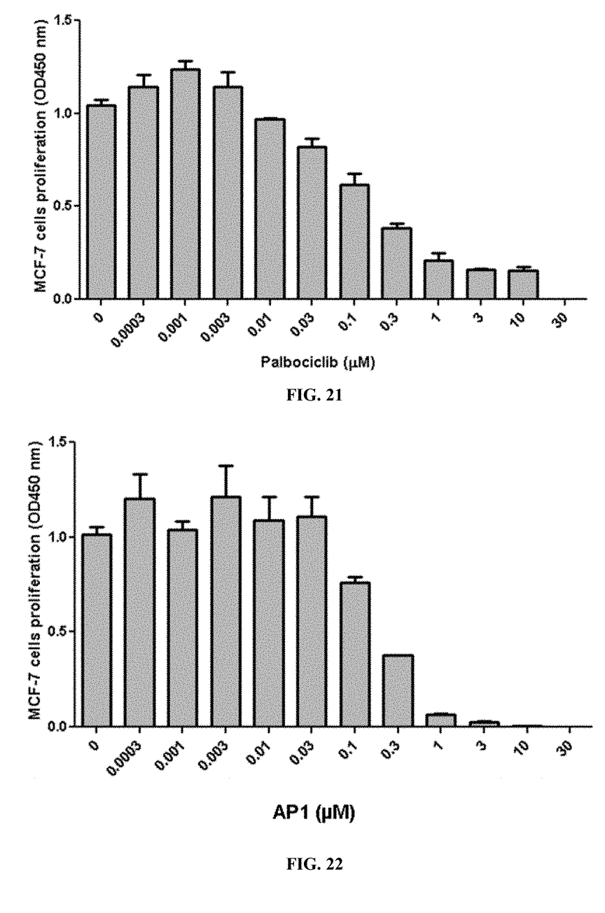

The combination of AP1 and palbociclib was tested at various drug doses on MCF-7 cells. Various MCF-7 cell numbers were plated and evaluated 3-7 days after plating to determine the optimal number of cells to be plated and to determine the treatment duration. The optimal number of cells were plated and treated with various concentrations of AP1 alone or with palbociclib alone. MCF-7 cells were evaluated for viability using the WST-1 assay or the CyQUANT method 3-7 days or 120 h after beginning treatment. FIG. 21 shows cell proliferation of MCF-7 cells when the cells were treated with palbociclib alone. FIG. 22 shows cell proliferation of MCF-7 cells when the cells were treated with AP1 alone. A number of concentrations around the IC₅₀ of AP1, and a number of concentrations around the IC₅₀ of palbociclib were determined. The EC₅₀ of AP1 on MCF-7 cells was determined to be 410 nM. The concentrations used to obtain the EC₅₀ value were tested on MCF-7 cells to test the effect of treatment with the combination of AP1 and palbociclib.

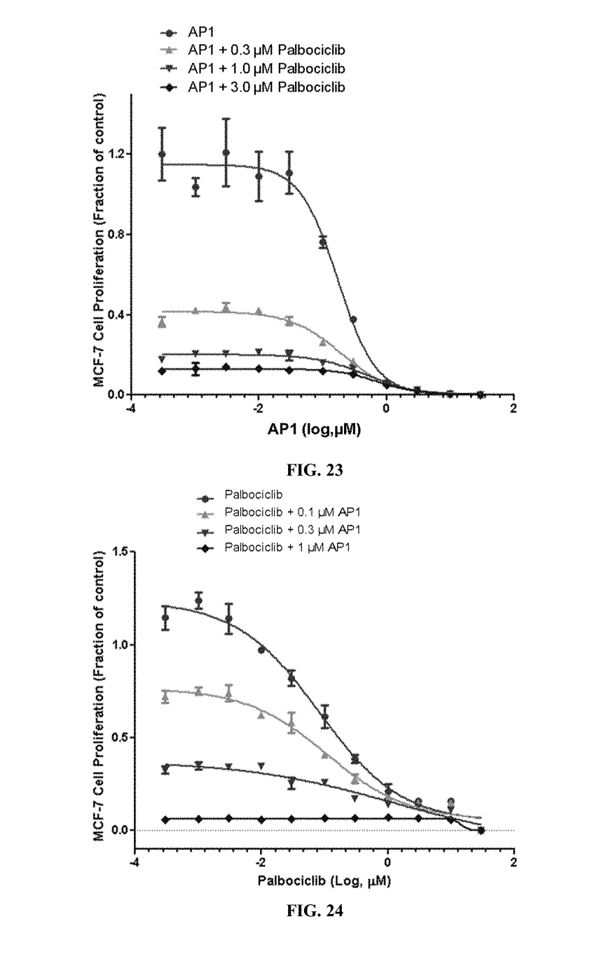

The optimal number of MCF-7 cells were plated and treated with AP1 and palbociclib. The MCF-7 cells were incubated for 3-5 days or 3-7 days. AP1 was added to the cells simultaneously with palbociclib, before adding palbociclib, or after the addition of palbociclib. The cells were evaluated for viability using the CyQUANT method after beginning the simultaneous treatments. FIG. 23 shows MCF-7 cell proliferation when the cells were treated simultaneously with a fixed amount of AP1 and varying amounts of palbociclib. FIG. 24 shows MCF-7 cell proliferation when the cells were treated simultaneously with a fixed amount of palbociclib and varying amounts of AP1.

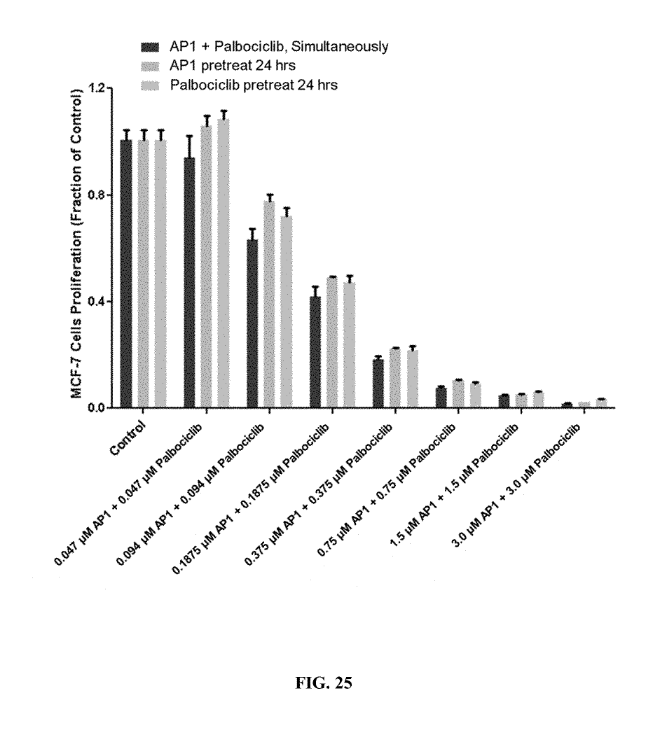

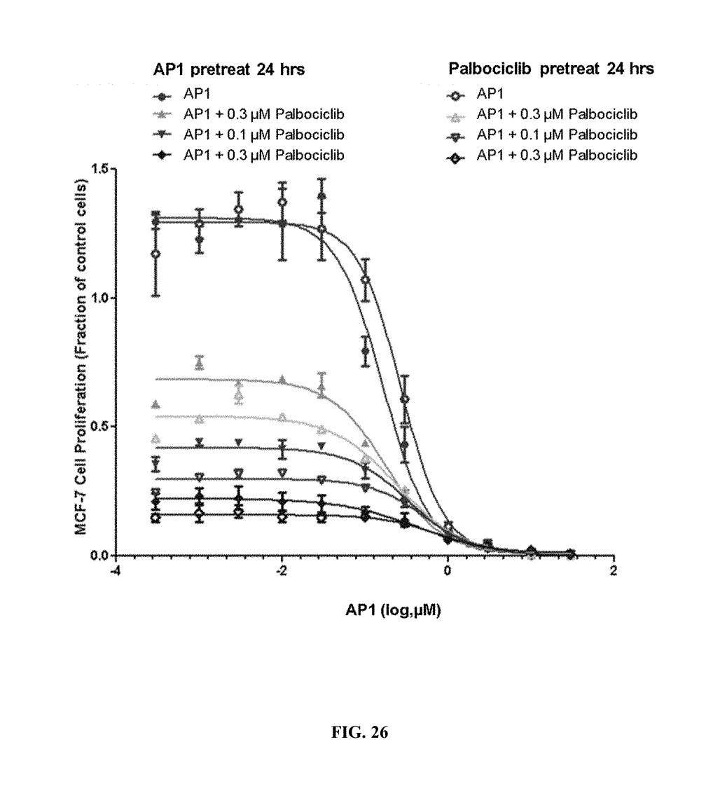

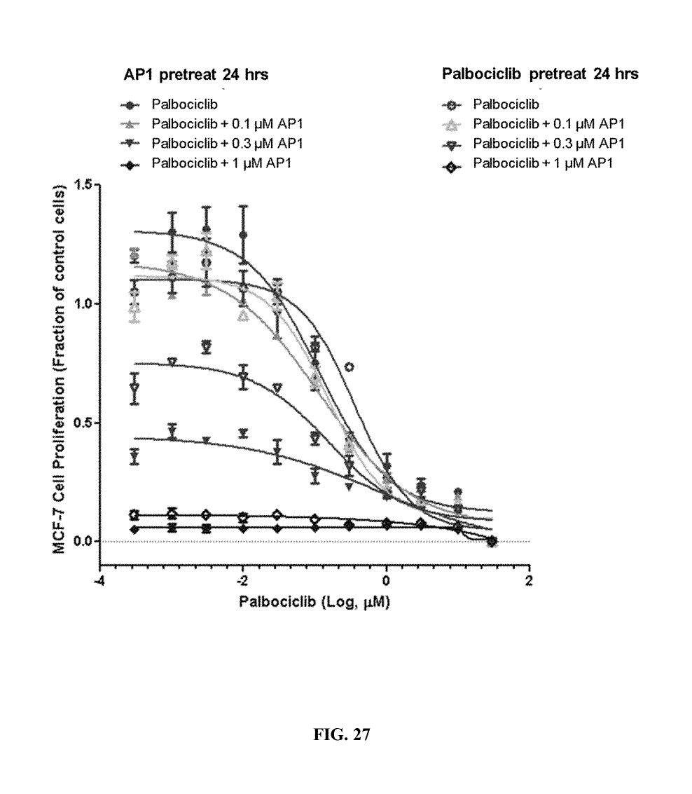

The cells were evaluated for viability using a WST-1 assay or MTT assay 3-7 days after beginning the treatments. The effects of adding AP1 and palbociclib in different orders was evaluated using various concentrations of AP1 using the CyQUANT method. FIG. 25 shows MCF-7 cell proliferation when the cells were treated with varying concentrations of AP1 and palbociclib in different orders over a period of 72 h. FIG. 26 shows MCF-7 cell proliferation when the cells were pre-treated with AP1 for 24 h and subsequently treated with varying concentrations of palbociclib; and when the cells were pre-treated with varying concentrations of palbociclib for 24 h and subsequently treated with a fixed amount of AP1. AP1 suppressed MCF-7 cell growth with or without treatment with palbociclib. FIG. 27 shows MCF-7 cell proliferation when the cells were pre-treated with varying concentrations of AP1 for 24 h and subsequently treated with fixed amounts of palbociclib; and when the cells were pre-treated with fixed amounts of palbociclib and subsequently treated with varying concentrations of AP1. Palbociclib suppressed MCF-7 cell growth with or without treatment with AP1.

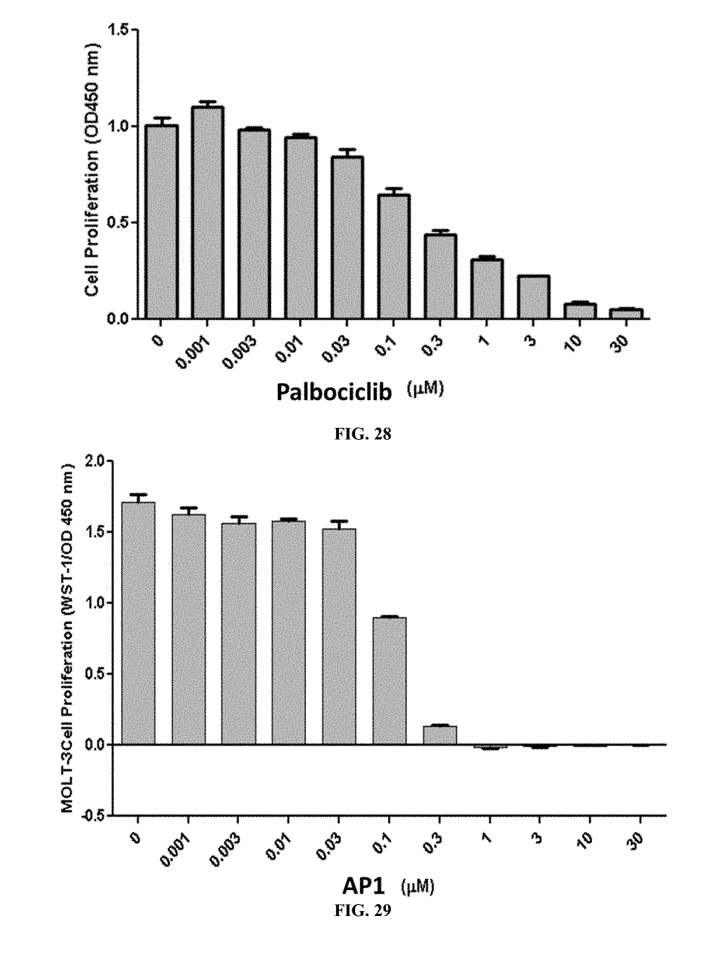

The combination of AP1 and palbociclib was tested at various drug doses on MOLT-3 cells. Various MOLT-3 cell numbers were plated and evaluated 3-7 days after plating to determine the optimal number of cells to be plated and to determine the treatment duration. The optimal number of cells were plated and treated with various concentrations of AP1 alone or with palbociclib alone. The MOLT-3 cells were evaluated for viability using the WST-1 assay or the CyQUANT method 3-7 days or 120 h after beginning treatment. FIG. 28 shows MOLT-3 cell proliferation when the cells were treated with palbociclib alone. FIG. 29 shows MOLT-3 cell proliferation when the cells were treated with AP1 alone.

Combination Index Plots of AP1 and Palbociclib Using WST-1 and CyQUANT Assays in MCF-7 Cells.

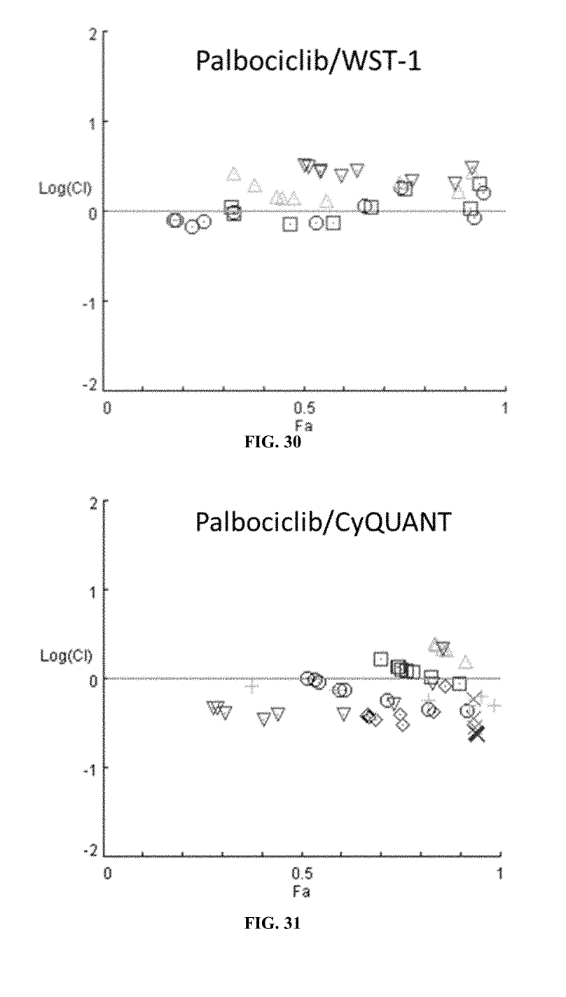

Combination index plots of treatment with AP1 and palbociclib in MCF-7 cells showed additive or increased complementarity. FIG. 30 shows the combination index plot of the treatment of MCF-7 cells with AP1 and palbociclib using a WST-1 assay. FIG. 31 shows the combination index plot of the treatment of MCF-7 cells with AP1 and palbociclib using CyQUANT. Example cooperativity index calculations are shown in TABLE 14. The data are expressed as log(CI). CI values: 0-0.1, very strong synergism; 0.1-0.3, strong synergism; 0.3-0.7, synergism; 0.7-0.85, moderate synergism; 0.85-0.90, slight synergism; 0.90-1.10, nearly additive; 1.10-1.20, slight antagonism; 1.20-1.45, moderate antagonism; 1.45-3.3, antagonism; 3.3-10, strong antagonism; 10, very strong antagonism.

TABLE 14

| Dose AP1 | Dose palbociclib | | |
|---|---|---|---|
| (μM) | (μM) | Effect | CI |
| 0.001 | 0.3 | 0.178 | 0.59570 |
| 0.003 | 0.3 | 0.184 | 0.59898 |
| 0.01 | 0.3 | 0.223 | 0.54530 |
| 0.03 | 0.3 | 0.25 | 0.62998 |
| 0.1 | 0.3 | 0.325 | 0.79278 |
| 0.3 | 0.3 | 0.532 | 0.68885 |
| 1.0 | 0.3 | 0.65 | 1.13080 |
| 3.0 | 0.3 | 0.743 | 1.92593 |
| 10.0 | 0.3 | 0.924 | 1.17267 |
| 30.0 | 0.3 | 0.945 | 2.32597 |
| 0.4 | 0.001 | 0.585 | 0.57898 |
| 0.4 | 0.003 | 0.553 | 0.67550 |
| 0.4 | 0.01 | 0.55 | 0.68802 |
| 0.4 | 0.03 | 0.545 | 0.71276 |
| 0.4 | 0.1 | 0.556 | 0.70459 |
| 0.4 | 0.3 | 0.608 | 0.61579 |
| 0.4 | 1.0 | 0.592 | 0.90805 |
| 0.4 | 3.0 | 0.614 | 1.46501 |
| 0.4 | 10.0 | 0.698 | 2.61449 |
| 0.4 | 30.0 | 0.999 | 0.02893 |

The efficacy of AP1 alone and in combination with palbociclib was tested in the SJSA-1 osteosarcoma xenograft model using female athymic nude mice. Charles River NCr nu/nu mice with 5×10⁶ SJSA-1 tumor cells in 0% Matrigel® were injected subcutaneously into the flank of the mice. The cell injection volume was 0.1 mL/mouse. The mice were 8-12 weeks of age at the beginning of the study. A pair match was performed when tumors reached an average size of 100 mm³-150 mm³, and the treatment regimen was started. Body weight and caliper measurements were made biweekly to the end of the study.

Any individual animal with a single observation of >30% body weight loss or three consecutive measurements of >25% body weight loss was euthanized. Any group with a mean body weight loss of >20% or >10% mortality was removed from the study. The group was not euthanized, and the mice were allowed to recover. Within a group with >20% weight loss, individuals hitting the individual body weight loss endpoint was euthanized. If the group treatment-related body weight loss was recovered to within 10% of the original weights, dosing was resumed at a lower dose or less frequent dosing schedule. Animals were monitored individually. The end point of the experiment was a tumor volume of 2000 mm³ or 60 days, whichever came first. Responders were followed for a longer period of time. When the endpoint was reached, the animals were euthanized.

Palbociclib was prepared as a solution in sodium lactate buffer (50 mM, pH 4.0). An aqueous phosphate-buffered saline solution or sodium lactate (50 mM, pH 4.0) solution was used as the vehicle. The dosing volume was 10 mL/kg (0.2 mL/20 g mouse), and the volume was adjusted according to the body weight of the mouse.

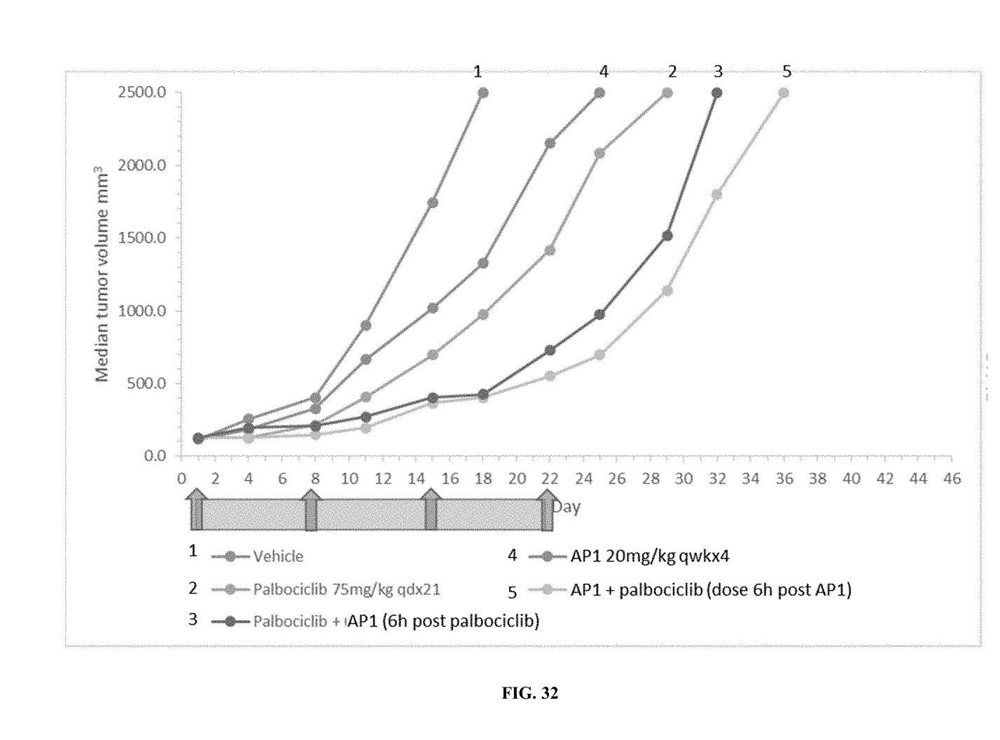

FIG. 32 shows the effects of AP1, palbociclib, or combination treatment with AP1+palbociclib on the median tumor volumes in the SJSA-1 osteosarcoma xenograft model. The data show that mice treated with a combination of AP1 and palbociclib required a longer duration to reach the same median tumor volume as mice treated with vehicle alone, AP1 alone, or palbociclib alone. Mice first treated with AP1 and treated with palbociclib 6 h after administration of AP1 required a longer duration to reach the same median tumor volume as mice first treated with palbociclib and treated with AP1 6 h after administration of palbociclib.

TABLE 11 shows the results of the CDK inhibitor efficacy test using combination treatment with AP1 and palbociclib in the SJSA-1 osteosarcoma xenograft model.

TABLE 11

| | Median | | | | | |
|---|---|---|---|---|---|---|
| | tumor | | % TGI | | Median time | Median time |
| | volume | | (±SEM) on | Animals | to tumor | to tumor |
| | (mm³) | | d22¹ (end | with disease | volume > 1000 | volume > 2000 |
| Treatment | D1 | D22 | of dosing) | progression² | mm³ (days) | mm³ (days) |
| Vehicle | 117 | 2500 | — | 10/10 | 12 | 16 |
| AP1 20 mg/kg | 117 | 2150 | 17 (6) | 10/10 | 16 | 21 |
| qwkx4 | | | | | | |
| Palbociclib 75 | 126 | 1418 | 51 (9) | 9/10 | 18 | 24 |
| mg/kg qdx22 | | | | | | |
| AP1 + | 126 | 550 | 82 (2) | 10/10 | 27 | 34 |
| Palbociclib | | | | | | |
| (dose 6 h post | | | | | | |
| AP1) | | | | | | |
| Palbociclib + | 126 | 727 | 71 (3) | 10/10 | 25 | 32 |
| AP1 (6 h post | | | | | | |
| palbociclib | | | | | | |

¹Calculated using vehicle median volumes on d0 and d22
²Defined as three consecutive measurements > 150% of d1 volume

The efficacy of AP1 alone and in combination with palbociclib was tested in the MCF-7.1 human breast carcinoma xenograft model using female athymic nude mice. Female athymic nude mice were provided with drinking water with 10 μg/mL with 17 beta estradiol supplementation 3 days prior to cell implantation and for the duration of the study. Charles River NCI athymic nude mice were treated with 1×10⁷ MCF-7.1 tumor cells in 0% Matrigel® subcutaneously in the flank. The cell injection volume was 0.1 mL/mouse. The mice were between 8-12 weeks of age at the beginning of the study. A pair match was performed when tumors reached an average size of 100 mm³-150 mm³, and the treatment regimen was started. Body weight and caliper measurements were made biweekly to the end of the study.

Any individual animal with a single observation of >30% body weight loss or three consecutive measurements of >25% body weight loss was euthanized. Any group with a mean body weight loss of >20% or >10% mortality was removed from the study. The group was not euthanized, and the mice were allowed to recover. Within a group with >20% weight loss, individuals hitting the individual body weight loss endpoint was euthanized. If the group treatment-related body weight loss was recovered to within 10% of the original weights, dosing was resumed at a lower dose or less frequent dosing schedule. Animals were monitored individually. The end point of the experiment was a tumor volume of 1000 mm³ or 60 days, whichever came first. Responders were followed for a longer period of time. When the endpoint was reached, the animals were euthanized.

Palbociclib was prepared as a solution in sodium lactate buffer (50 mM, pH 4.0). An aqueous phosphate-buffered saline solution or sodium lactate (50 mM, pH 4.0) solution was used as the vehicle. The dosing volume was 10 mL/kg (0.2 mL/20 g mouse), and the volume was adjusted according to the body weight of the mouse.

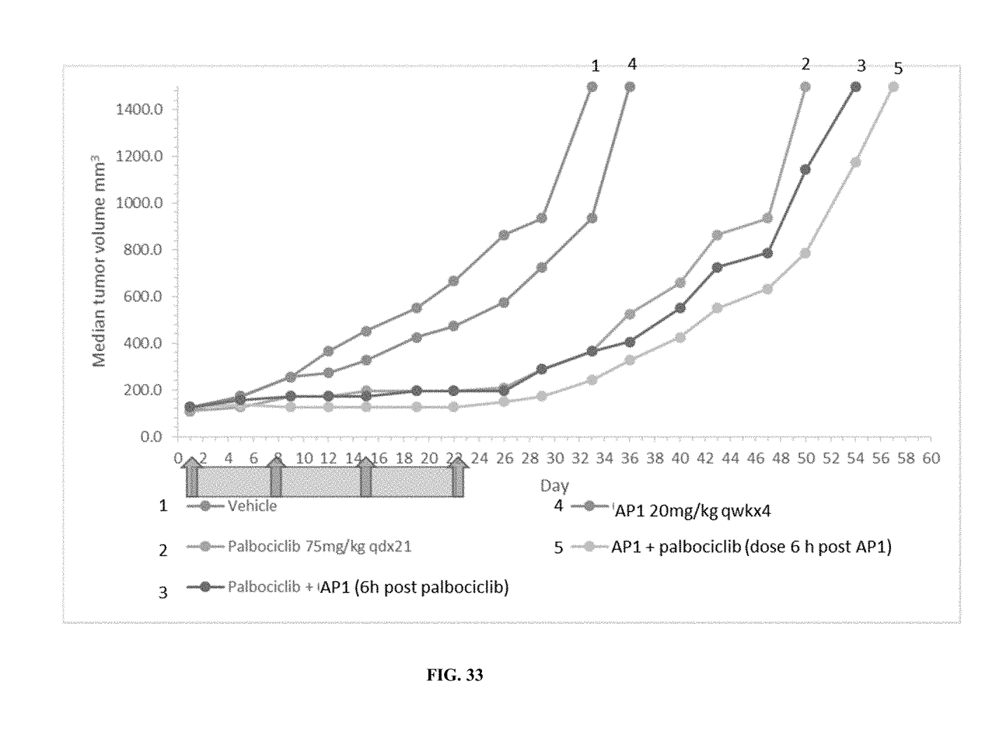

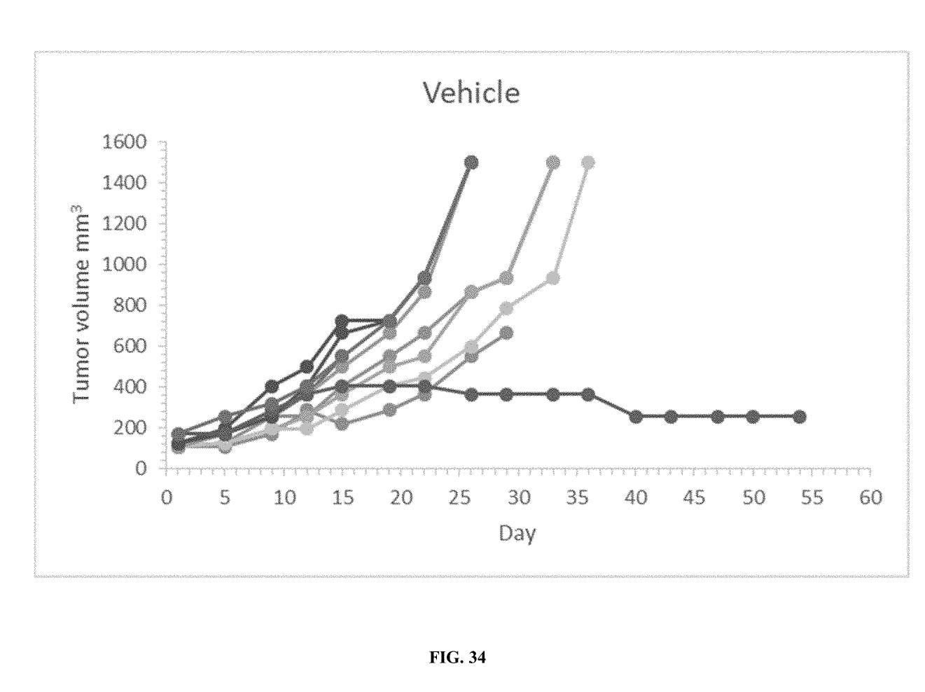

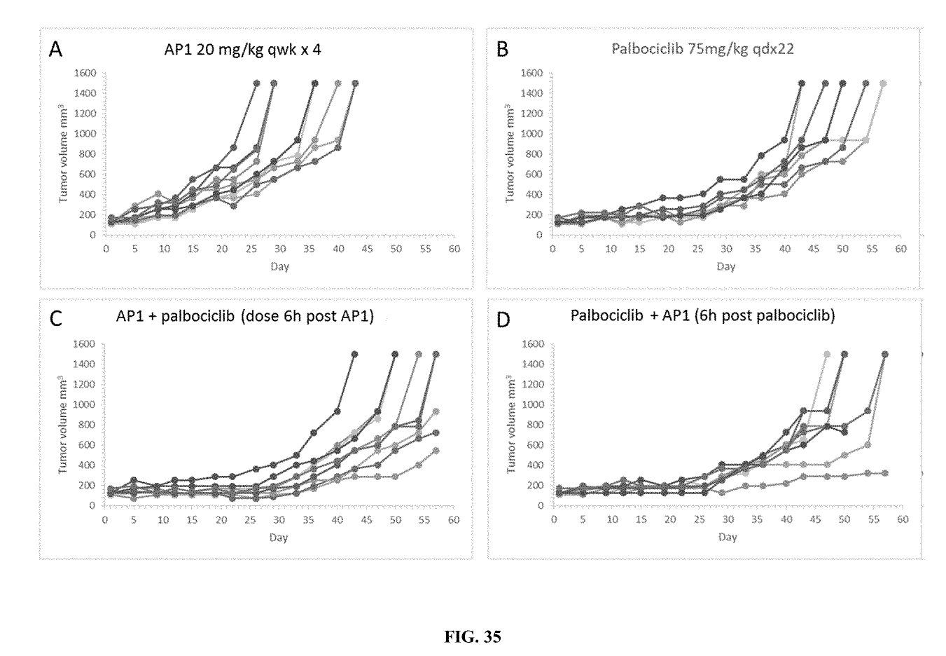

FIG. 33 shows the effects of AP1, palbociclib, or combination treatment with AP1+palbociclib on the median tumor volumes in the MCF-7.1 human breast carcinoma xenograft model. The data show that mice treated with a combination of AP1 and palbociclib required a longer duration to reach the same median tumor volume as mice treated with vehicle alone, AP1 alone, or palbociclib alone. FIG. 34 shows individual tumor volumes of mice treated with MCF-7.1 human breast carcinoma xenografts treated with the vehicle. FIG. 35 PANEL A shows the individual tumor volumes of mice treated with AP1 20 mg/kg qwk×4. FIG. 35 PANEL B shows the individual tumor volumes of mice treated with palbociclib 75 mg/kg qd×22. FIG. 35 PANEL C shows the individual tumor volumes of mice treated with AP1, and treated with palbociclib 6 h after administration of AP1. FIG. 35 PANEL D shows the individual tumor volumes of mice treated with palbociclib, and treated with AP1 6 h after administration of AP1. The data show that mice treated with a combination of AP1 and palbociclib required a longer duration to reach the same median tumor volume as mice treated with AP1 alone or palbociclib alone.

TABLE 12 shows the results of the CDK inhibitor efficacy test using the MCF-7.1 human breast carcinoma xenograft model.

TABLE 12

| | Median | | | | | |
|---|---|---|---|---|---|---|
| | tumor | | % TGI | | Median time | Median time |
| | volume | | (±SEM) on | Animals | to tumor | to tumor |
| | (mm³) | | d22¹ (end | with disease | volume > 500 | volume > 1000 |
| Treatment | D1 | D22 | of dosing) | progression² | mm³ (days) | mm³ (days) |
| Vehicle | 108 | 666 | — | 10/10 | 19 | 27 |
| | | | | | | 9/10 animals |
| | | | | | | have reached |
| | | | | | | endpoint |
| AP1 20 mg/kg | 126 | 473 | 30 (9) | 10/10 | 22 | 35 |
| qwkx4 | | | | | | 10/10 animals |
| | | | | | | have reached |
| | | | | | | endpoint |
| Palbociclib 75 | 108 | 196 | 84 (4) | 8/8 | 37 | 48 |
| mg/kg qdx22 | | | | | | 10/10 animals |
| | | | | | | have reached |
| | | | | | | endpoint |
| AP1 + | 126 | 126 | 95 (3) | 10/10 | 42 | 53 |
| Palbociclib | | | | | | 7/10 animals |
| (dose 6 h post | | | | | | have reached |
| AP1) | | | | | | endpoint |
| Palbociclib + | 126 | 196 | 88 (2) | 9/9 | 37 | 49 |
| AP1 (6 h post | | | | | | 6/7 animals |
| palbociclib | | | | | | have reached |
| | | | | | | endpoint |

¹Calculated using vehicle median volumes on d0 and d22
²Defined as three consecutive measurements > 150% of d1 volume

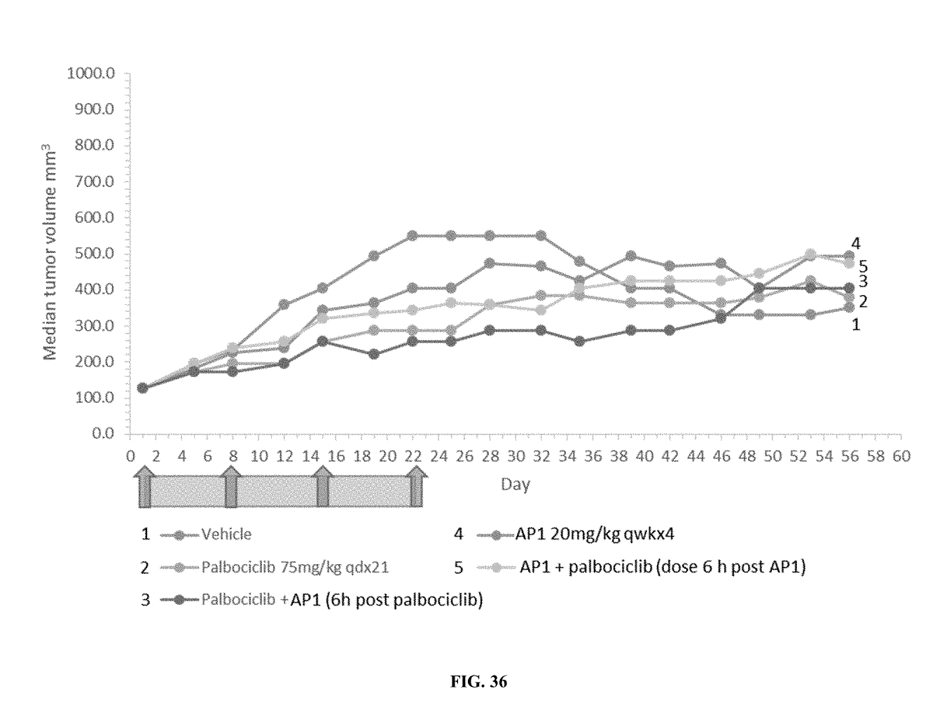

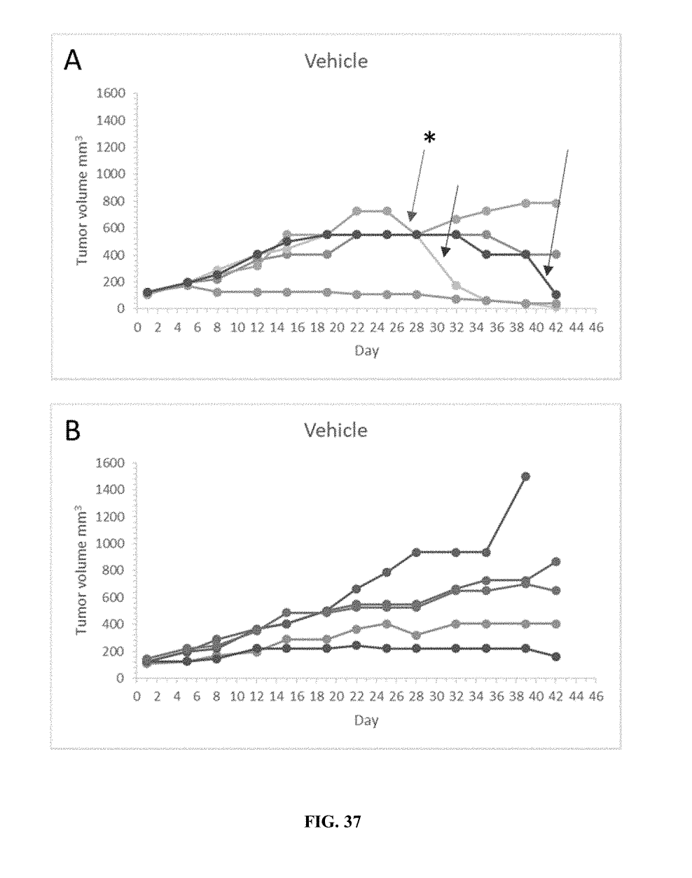

The efficacy of AP1 alone and in combination with palbociclib was tested in the A549 xenograft model using female athymic nude mice with the methods described above. FIG. 36 shows the effects of AP1, palbociclib, or combination treatment with AP1+palbociclib on the median tumor volumes in the A549 xenograft model. FIG. 37 PANEL A shows the effect of vehicle treatment on median tumor volumes in the A549 xenograft model. FIG. 37 PANEL B shows the effect of vehicle treatment on median tumor volumes in the A549 xenograft model. The arrows indicate spontaneous tumor shrinkage in vehicle controls. The arrows with * indicate poor growth of tumors late in the study.

TABLE 13 shows the CDK inhibitor efficacy test in the A549 xenograft model.

TABLE 13

| | Median | | | | | |
|---|---|---|---|---|---|---|
| | tumor | | % TGI | | Median time | Median time |
| | volume | | (±SEM) on | Animals | to tumor | to tumor |
| | (mm³) | | d22¹ (end | with disease | volume > 500 | volume > 1000 |
| Treatment | D1 | D22 | of dosing) | progression² | mm³ (days) | mm³ (days) |
| Vehicle | 126 | 500 | — | 9/10 | 18 | ND |
| | | | | | 9/10 animals | 1/10 animals |
| | | | | | have reached | have reached |
| | | | | | endpoint | endpoint |
| AP1 20 mg/kg | 126 | 405 | 42 (7) | 10/10 | 25 | ND |
| qwkx4 | | | | | 8/10 animals | |
| | | | | | have reached | |
| | | | | | endpoint | |
| Palbociclib 75 | 126 | 288 | 62 (7) | 9/10 | 5/10 animals | ND |
| mg/kg qdx22 | | | | | have reached | |
| | | | | | endpoint | |
| AP1 + | 126 | 343 | 48 (7) | 10/10 | 6/10 animals | ND |
| Palbociclib | | | | | have reached | |
| (dose 6 h post | | | | | endpoint | |
| AP1) | | | | | | |
| Palbociclib + | 126 | 256 | 77 (9) | 7/9 | 4/9 animals | ND |
| AP1 (6 h post | | | | | have reached | |
| palbociclib | | | | | endpoint | |

¹Calculated using vehicle median volumes on d0 and d22
²Defined as three consecutive measurements > 150% of d1 volume

### Example 28: Combination Therapy with AP1 and MEK Inhibitors

a. Combination Therapy with AP1 and Trametinib

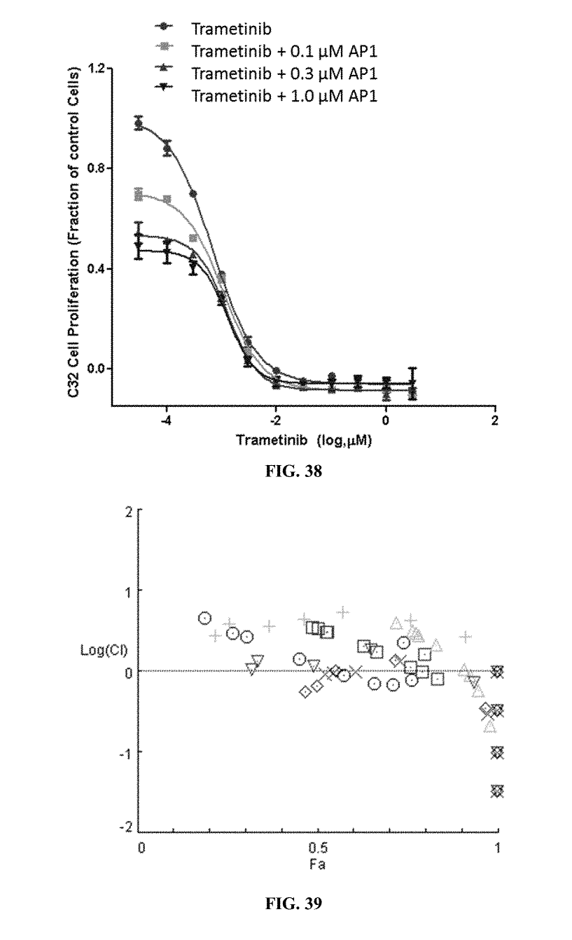

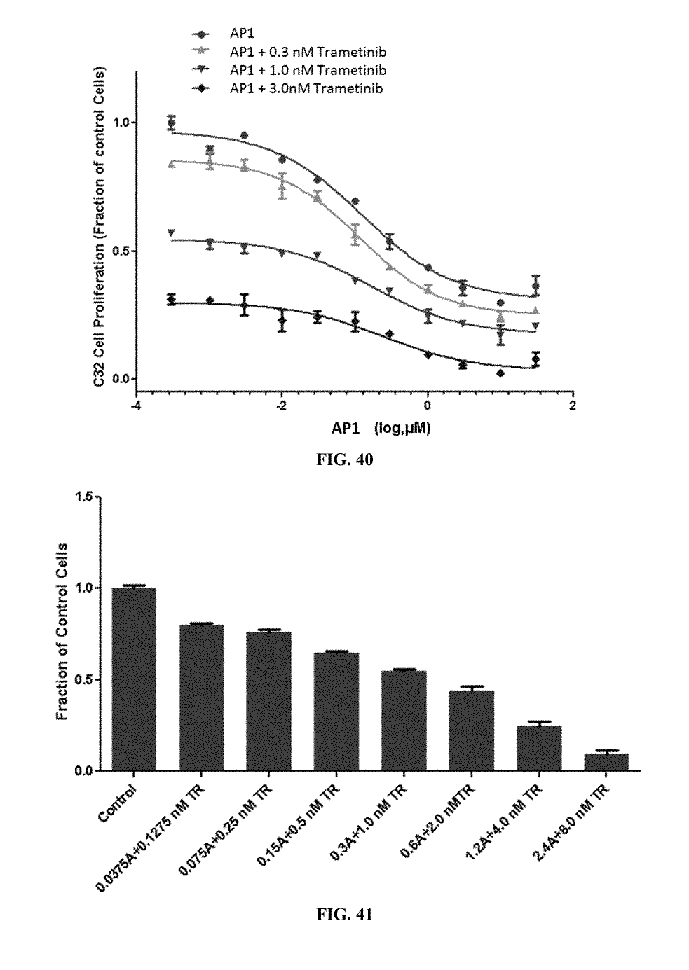

The combination of AP1 and trametinib was tested on human melanoma tumor C32 cells. FIG. 38 shows C32 cell proliferation when the cells were treated with trametinib alone or trametinib in combination with varying concentrations of AP1. FIG. 39 shows the combination index plot of the treatment of C32 cells with AP1 and trametinib. FIG. 40 shows C32 cell proliferation when the cells were treated with AP1 alone or AP1 with varying concentrations of trametinib. FIG. 41 shows C32 cell proliferation when the cells were treated with varying concentrations of AP1 and varying concentrations of trametinib.

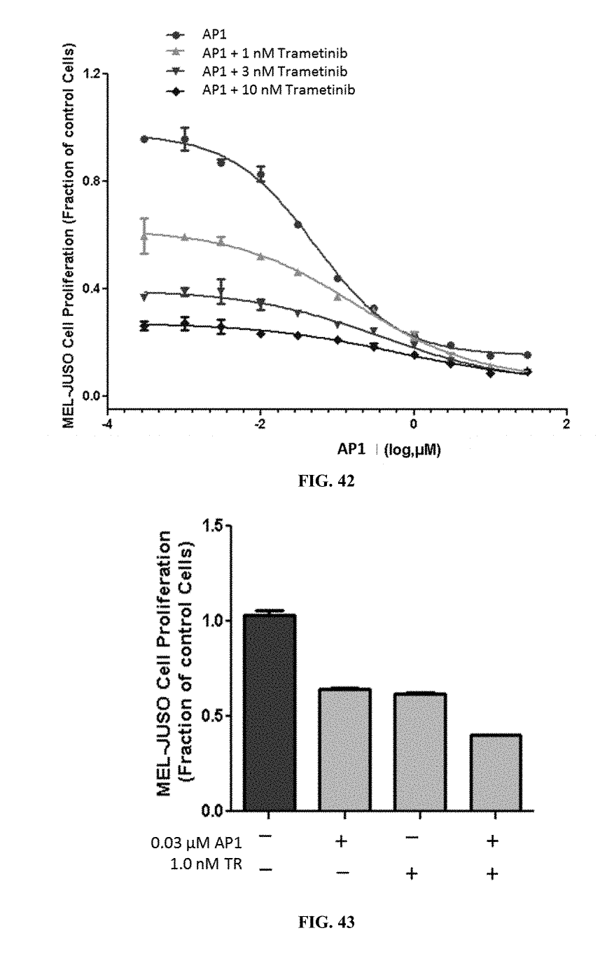

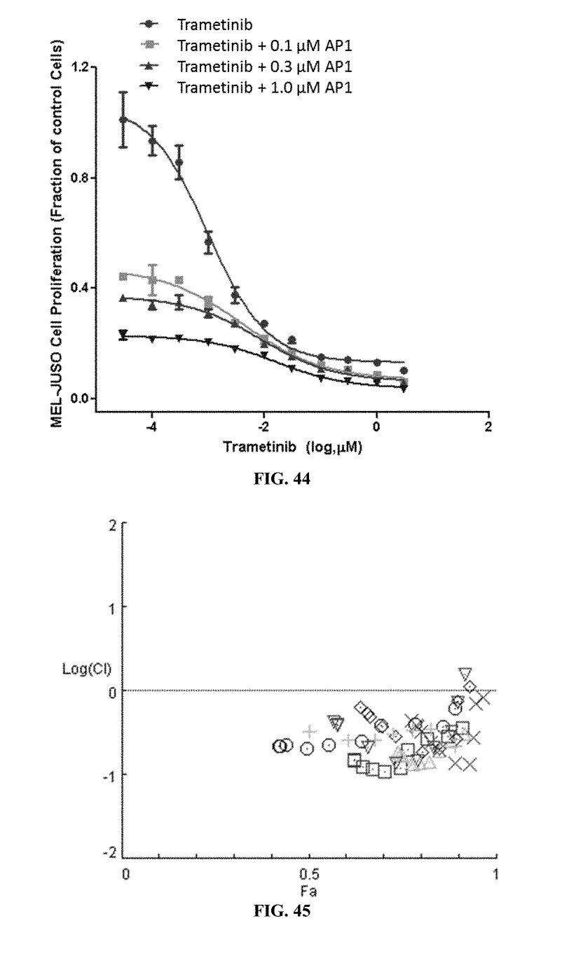

The combination of AP1 and trametinib was tested on MEL-JUSO cells. FIG. 42 shows MEL-JUSO cell proliferation when the cells were treated with AP1 alone or AP1 and varying concentrations of trametinib. FIG. 43 shows MEL-JUSO cell proliferation when the cells were treated with no agent, AP1 alone, trametinib alone, or 0.03 μM AP1 and 1.0 nM trametinib. FIG. 44 shows MEL-JUSO cell proliferation when the cells were treated with trametinib alone or trametinib with varying concentrations of AP1. FIG. 45 shows the combination index plot of the treatment of MEL-JUSO cells with AP1 and trametinib.

The combination of AP1 and trametinib was tested on A375 human melanoma cells. Various A375 cell numbers were plated and evaluated 3-7 later to determine the optimal number of cells to be treated and to determine the optimal treatment duration. The optimal number of cells were plated and treated with various concentrations of AP1 alone or trametinib alone. The cells were evaluated for viability using a WST-1 assay or MTT assay 3-7 days after treatment. A number of concentrations around the IC₅₀ of AP1 and a number of concentrations around the IC₅₀ of trametinib were determined. The EC₅₀ of AP1 on A375 cells was 70 nM.

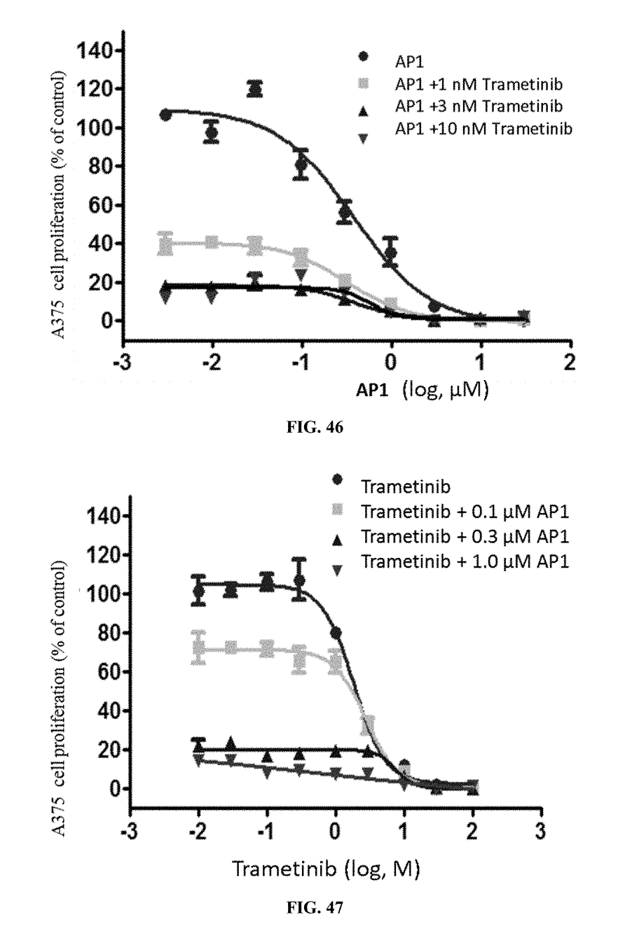

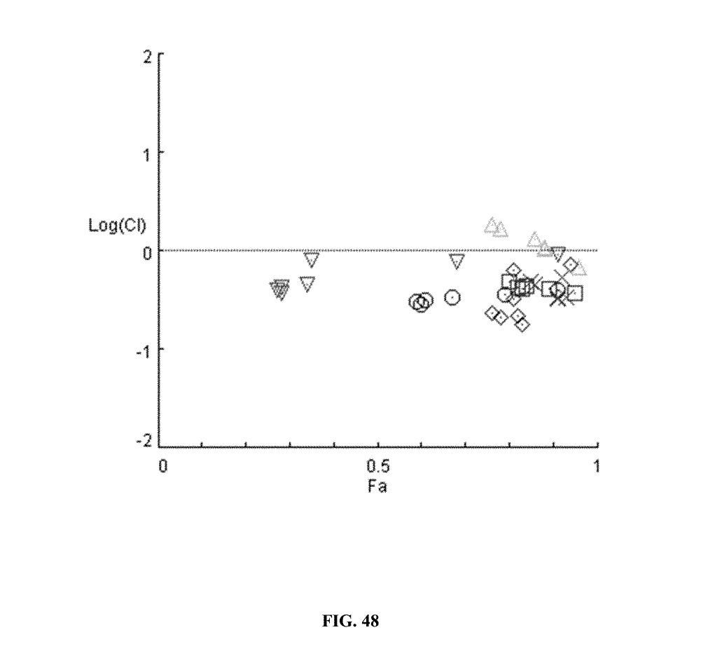

The cell viability of A375 cells were tested against treatment with AP1 at the selected concentrations in combination with trametinib. The optimal number of A375 cells was plated, and the cells were treated with AP1 and trametinib. The cells were evaluated for viability using a WST-1 assay or MTT assay 3-7 days after beginning simultaneous or sequential treatments with AP1 and trametinib. FIG. 46 shows A375 cell proliferation when the cells were treated with AP1 alone or AP1 in combination with varying concentrations of trametinib. FIG. 47 shows A375 cell proliferation when the cells were treated with trametinib alone or trametinib in combination with varying concentrations of AP1. FIG. 48 shows the combination index plot of the treatment of A375 melanoma cells with AP1 and trametinib.

b. Combination Therapy with AP1 and Binimetinib

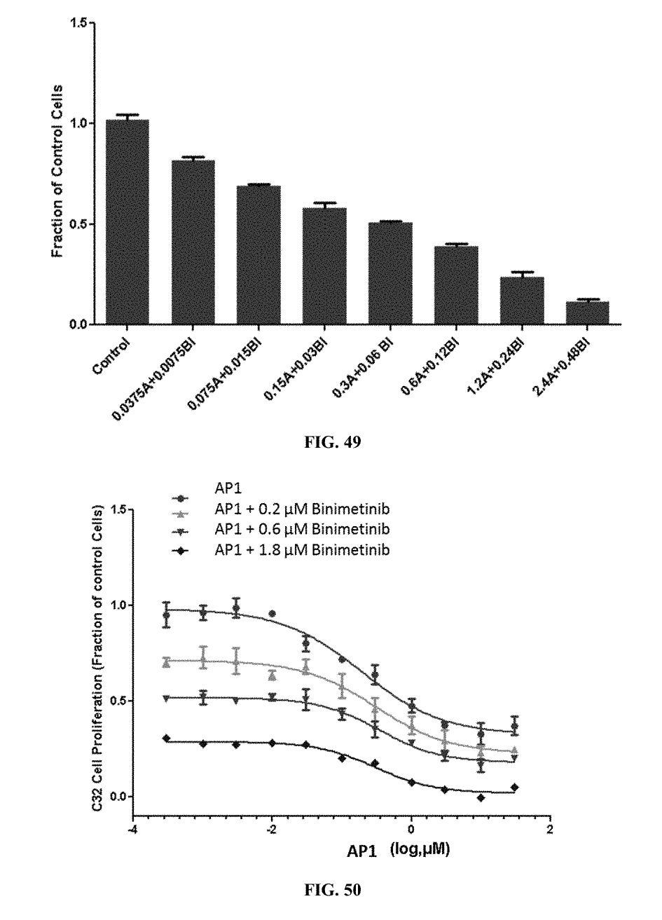

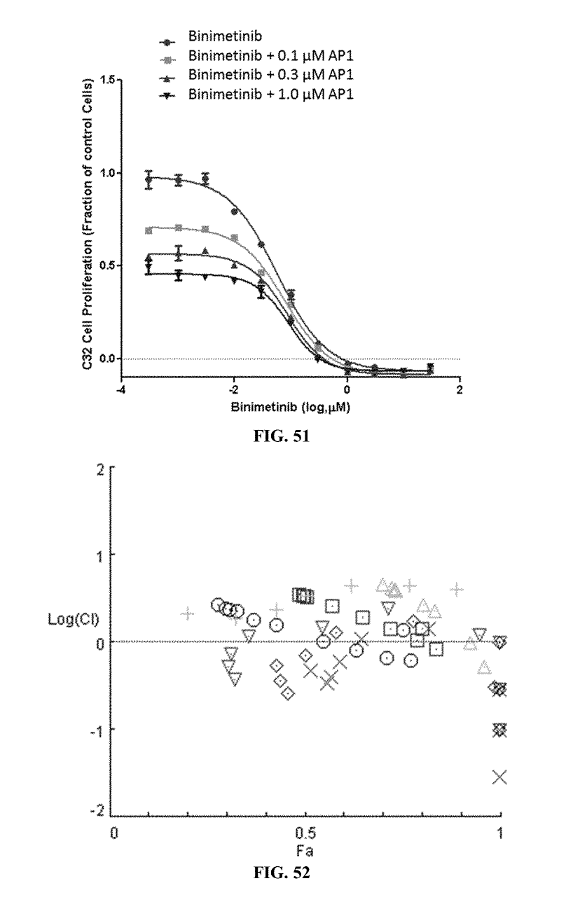

The combination of AP1 and binimetinib was tested on human melanoma tumor C32 cells. The C32 cells were grown in EMEM medium supplemented with 10% (v/v) fetal calf serum, 100 units of penicillin, and 100 μg/mL of streptomycin at 37° C. and 5% CO₂. One day prior to performing the assay, the C32 cells were trypsinized, counted, and seeded at 3000 cells/well/200 μL in 96-well plates. The cells were dosed with AP1 alone, binimetinib alone, or AP1 and binimetinib. The cells were incubated for 72 h, and cell viability was measured using a WST-1 variant of the MTT assay. FIG. 49 shows C32 cell proliferation when the cells were treated with varying concentrations of binimetinib and AP1. FIG. 50 shows C32 cell proliferation when the cells were treated with AP1 alone or AP1 in combination with varying concentrations of binimetinib. FIG. 51 shows C32 cell proliferation when the cells were treated with binimetinib alone or binimetinib in combination with varying concentrations of AP1. FIG. 52 shows the combination index plot of the treatment of C32 cells with AP1 and binimetinib. The combination index plot showed additive or increased complimentarily for treatment with AP1 and binimetinib in C32 cells.

The combination of AP1 and binimetinib was tested on MEL-JUSO cells. MEL-JUSO cells were grown in EMEM medium supplemented with 10% (v/v) fetal calf serum, 100 units of penicillin, and 100 μg/mL of streptomycin at 37° C. and 5% CO₂. One day prior to performing the assay, the MEL-JUSO cells were trypsinized, counted, and seeded at 3000 cells/well/200 μL in 96-well plates. The cells were dosed with AP1 alone, binimetinib alone, or AP1 and binimetinib. The cells were incubated for 72 h, and cell viability was measured using a WST-1 variant of the MTT assay.

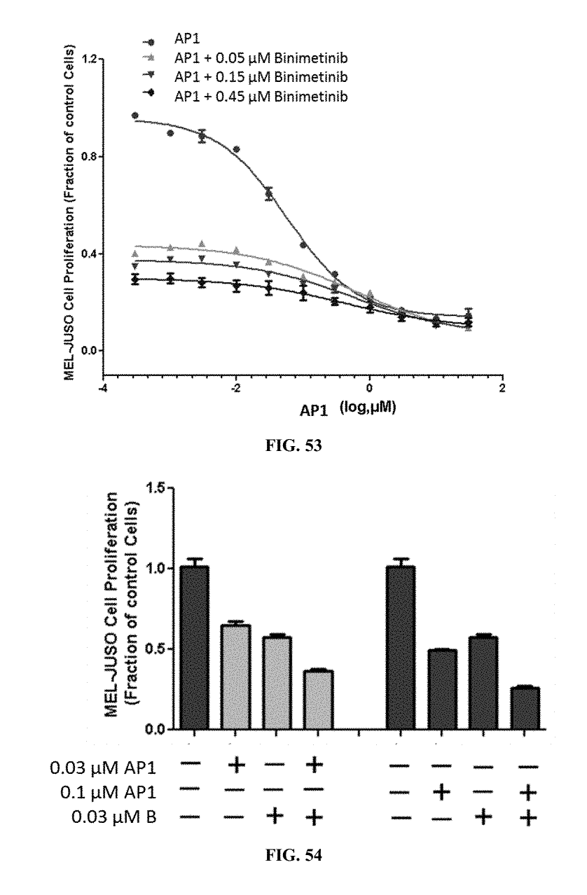

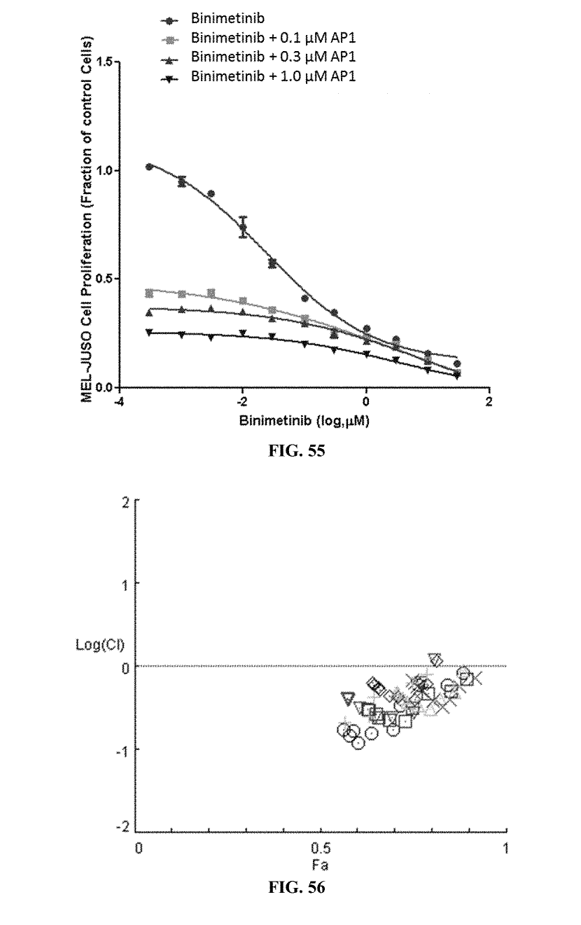

FIG. 53 shows MEL-JUSO cell proliferation when the cells were treated with AP1 alone or AP1 in combination with varying concentrations of binimetinib. FIG. 54 shows MEL-JUSO cell proliferation when the cells were treated with AP1 alone or AP1 in combination with varying concentrations of binimetinib. FIG. 55 shows MEL-JUSO cell proliferation when the cells were treated with binimetinib alone or binimetinib in combination with varying concentrations of AP1. FIG. 56 shows the combination index plot of the treatment of MEL-JUSO cells with AP1 and binimetinib. The combination index plot showed additive or increased complimentarily for treatment with AP1 and binimetinib in MEL-JUSO cells.

c. Combination Therapy with AP1 and Pimasertib

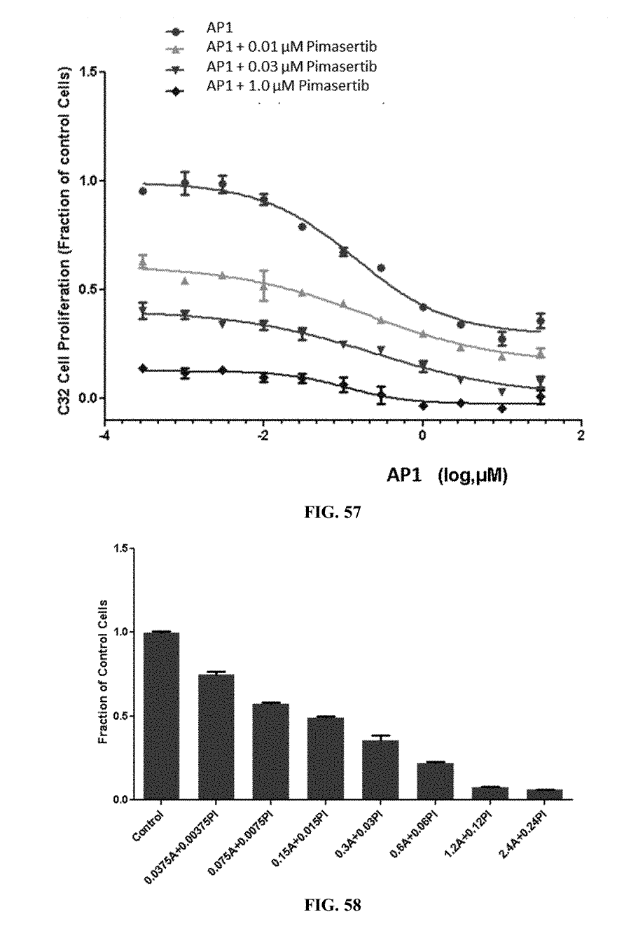

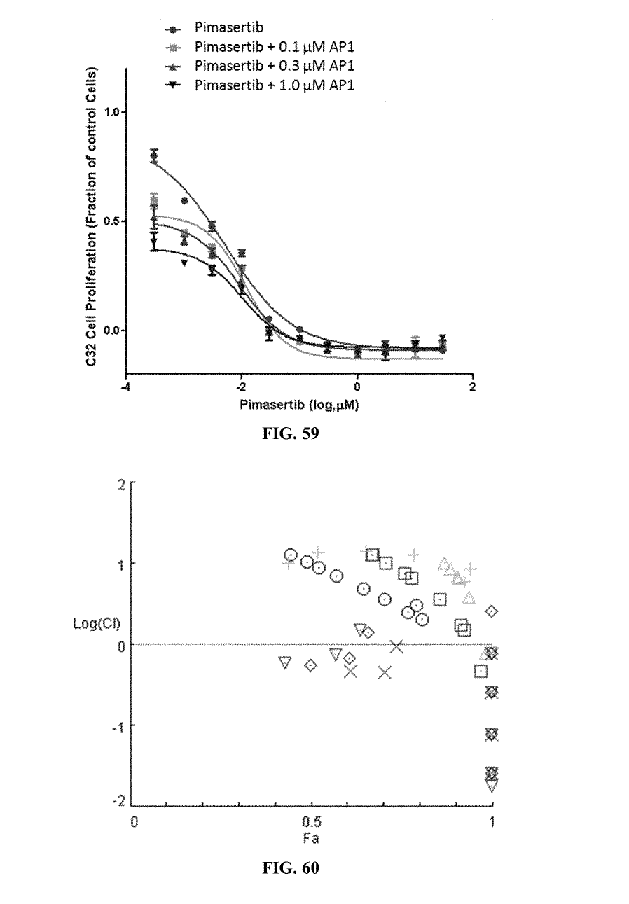

The combination of AP1 and pimasertib was tested on human melanoma tumor C32 cells. FIG. 57 shows C32 cell proliferation when the cells were treated with AP1 alone or AP1 in combination with varying combinations of pimasertib. FIG. 58 shows C32 cell proliferation when the cells were treated with varying concentrations of AP1 and pimasertib. FIG. 59 shows C32 cell proliferation when the cells were treated with pimasertib alone or pimasertib in combination with varying concentrations of AP1. FIG. 60 shows the combination index plot of the treatment of C32 cells with AP1 and pimasertib.

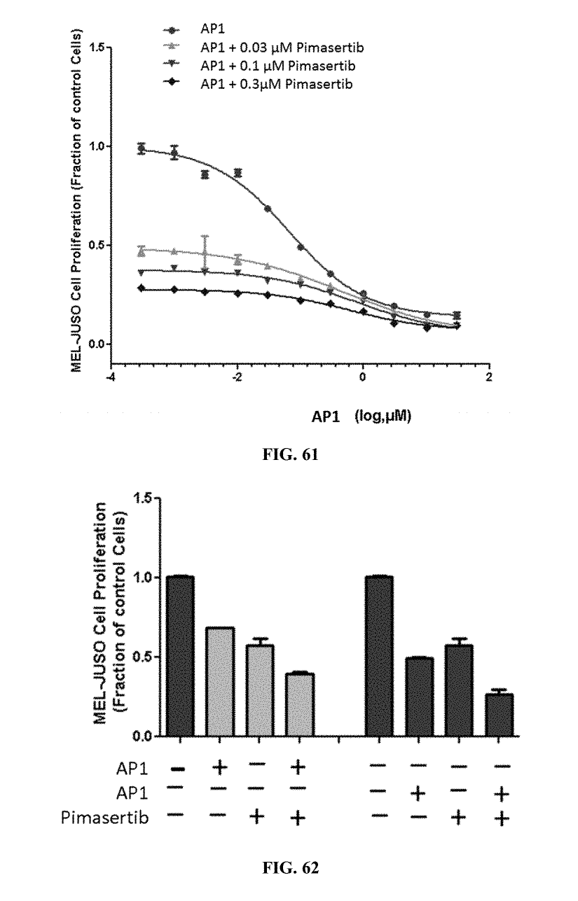

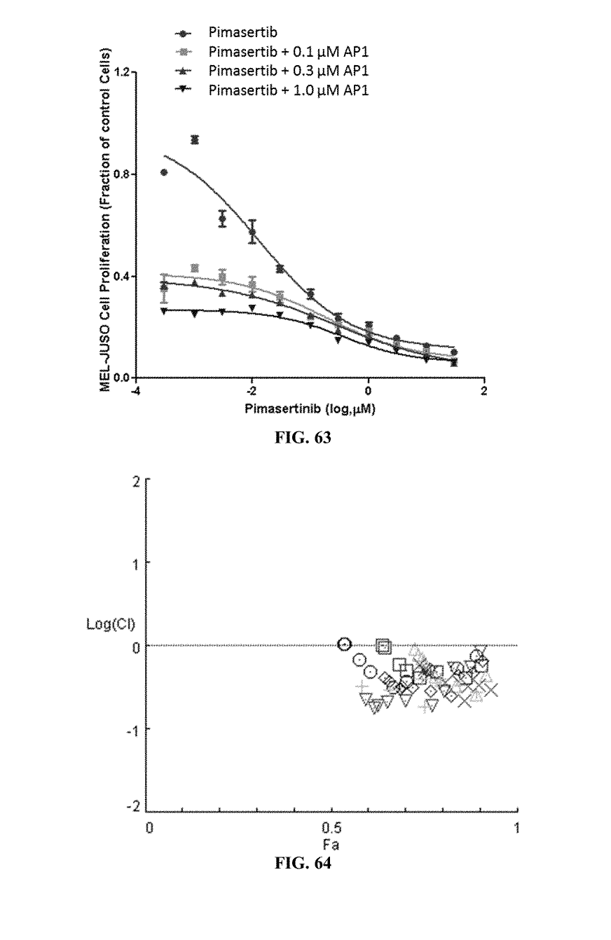

The combination of AP1 and pimasertib was tested on MEL-JUSO cells. FIG. 61 shows MEL-JUSO cell proliferation when the cells were treated with AP1 alone or AP1 in combination with varying concentrations of pimasertib. FIG. 62 shows MEL-JUSO cell proliferation when the cells were treated with AP1 and pimasertib. FIG. 63 shows MEL-JUSO cell proliferation when the cells were treated with pimasertib alone or pimasertib in combination with varying concentrations of AP1. FIG. 64 shows the combination index plot of the treatment of MEL-JUSO cells with AP1 and pimasertib.

d. Combination Therapy with AP1 and Selumetinib

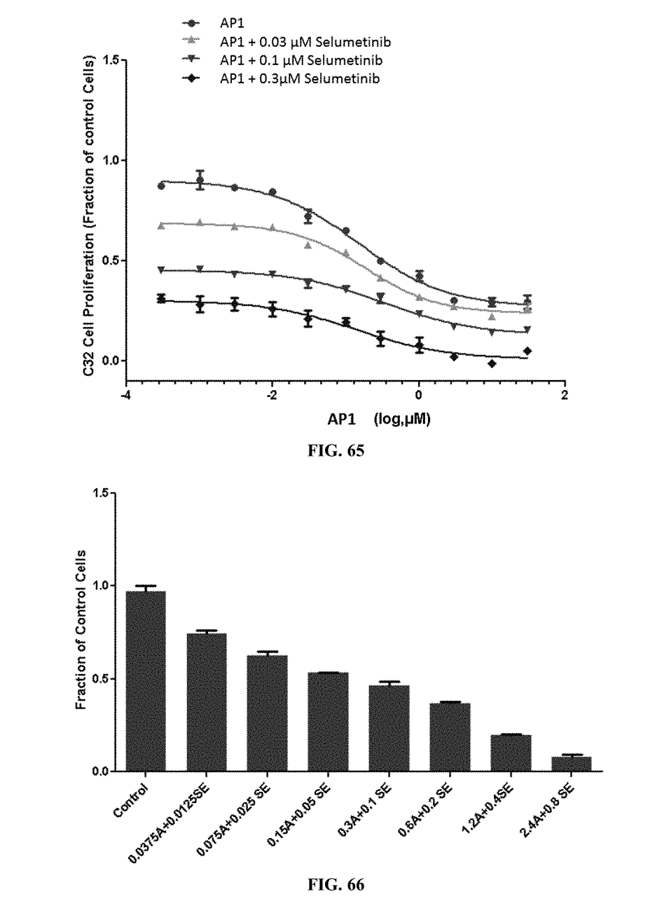

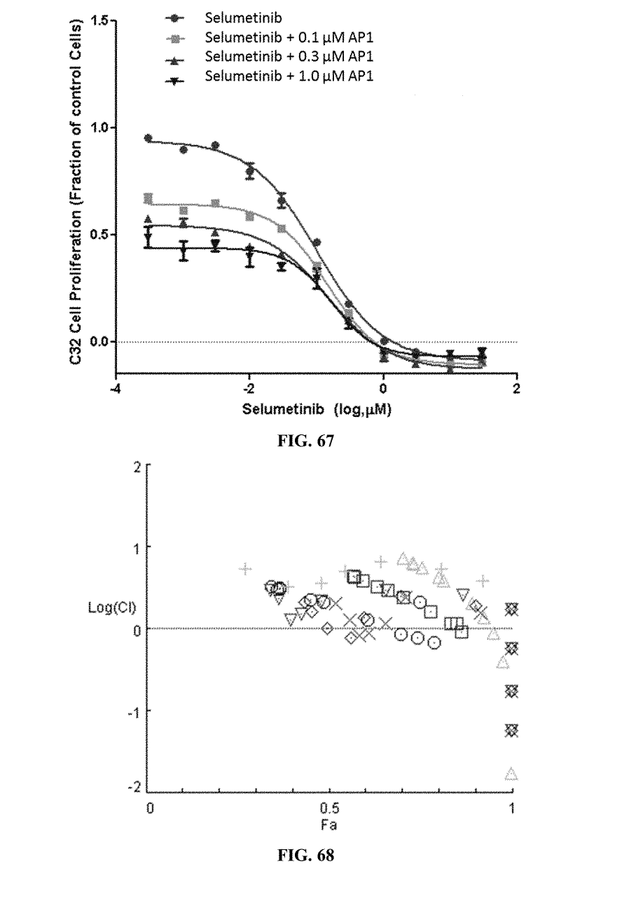

The combination of AP1 and selumetinib was tested on human melanoma tumor C32 cells. FIG. 65 shows C32 cell proliferation when the cells were treated with AP1 alone or AP1 in combination with varying combinations of selumetinib. FIG. 66 shows C32 cell proliferation when the cells were treated with varying concentrations of AP1 and selumetinib. FIG. 67 shows C32 cell proliferation when the cells were treated with selumetinib alone or selumetinib in combination with varying concentrations of AP1. FIG. 68 shows the combination index plot of the treatment of C32 cells with AP1 and selumetinib.

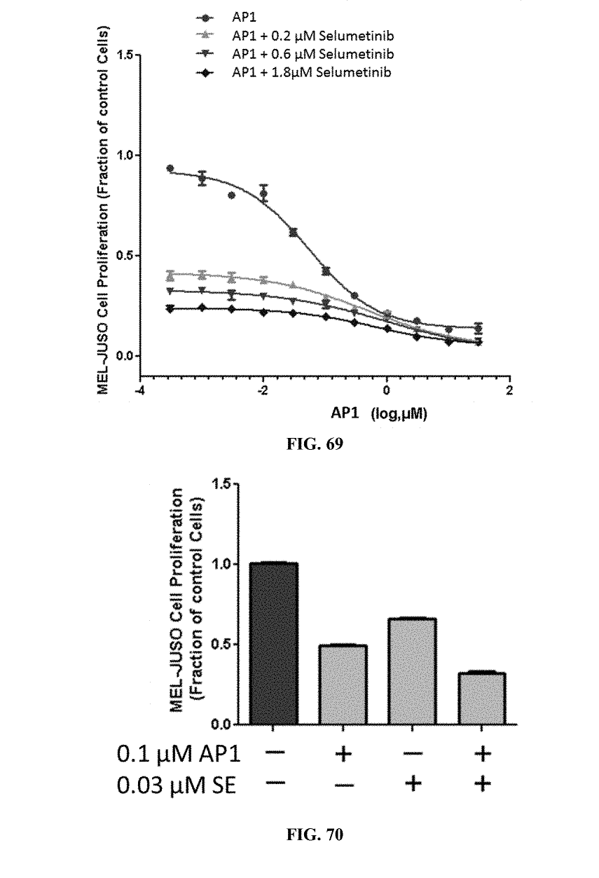

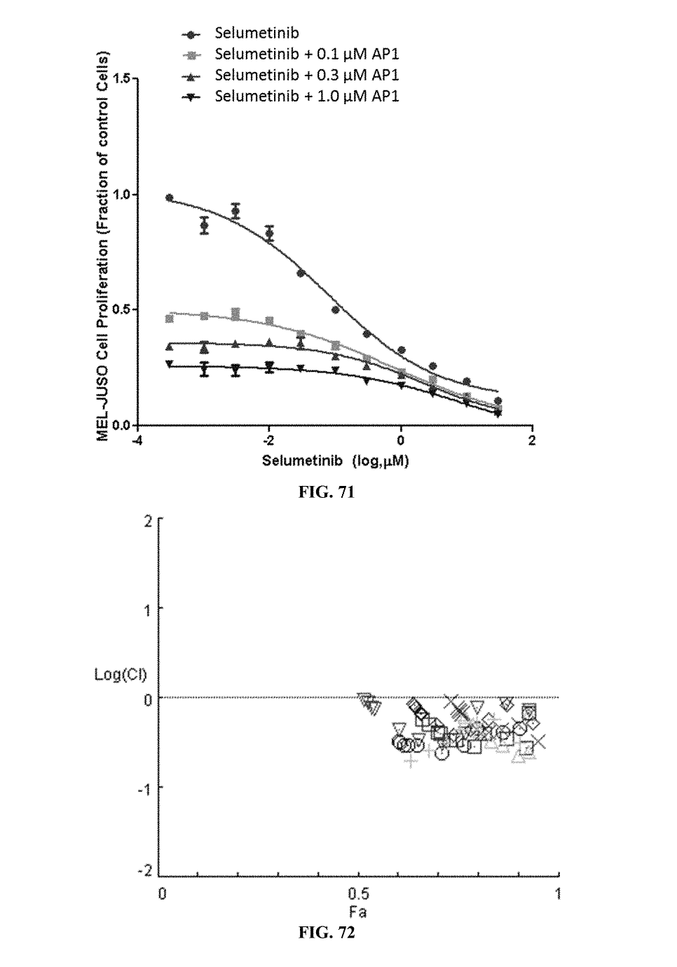

The combination of AP1 and pimasertib was tested on MEL-JUSO cells. FIG. 69 shows MEL-JUSO cell proliferation when the cells were treated with AP1 alone or AP1 in combination with varying concentrations of pimasertib. FIG. 70 shows MEL-JUSO cell proliferation when the cells were treated with AP1 and pimasertib. FIG. 71 shows MEL-JUSO cell proliferation when the cells were treated with pimasertib alone or pimasertib in combination with varying concentrations of AP1. FIG. 72 shows the combination index plot of the treatment of MEL-JUSO cells with AP1 and pimasertib.

### Example 29: Combination Therapy with AP1 and Cancer Agents

a. Clinical Development for the Treatment of Acute Myeloid Leukemia

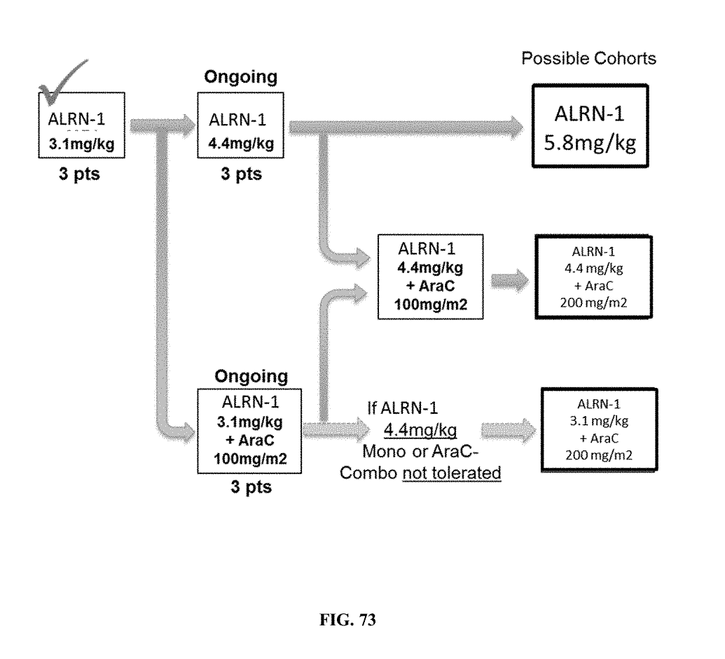

AP1 was tested for the treatment of patients with the hematological cancers, Acute Myeloid Leukemia (AML) or Myelodysplastic Syndrome (MDS), expressing WT p53. AML and MDS patients received AP1 or AP1 in combination with cytarabine. Cytarabine is an important agent for the treatment of patients with AML or MDS. Combination treatment is a standard treatment practice in oncology used to improve patient outcomes. FIG. 73 shows combination treatment and dosing regimens used to study the effects of AP1 to treat AML.

TABLE 14 shows initial patient analyses of the AML study. Of the evaluable patients where bone marrow biopsies were available before and after dosing with AP1, 3 patients demonstrated stabilization of their disease.

TABLE 14

| | | | | Days on | Best overall |
|---|---|---|---|---|---|
| Patient # | Disease | Dose level | Status | treatment | response |
| 1 | MDS | 3.1 alone | Disease | 98 | SD |
| | | | progression | | |
| 2 | MDS | 3.1 alone | Ongoing | 138 | SD |
| 3 | AML | 3.1 alone | Withdrew | 99 | SD |
| | | | consent | | |
| 4 | AML | 3.1 combo | Disease | 68 | ODP |
| | | | progression | | |
| 5 | AML | 3.1 combo | Ongoing | 41 | tbd |
| 6 | AML | 3.1 combo | Ongoing | 19 | tbd |
| 7 | MDS | 4.4 alone | Ongoing | 82 | tbd |
| 8 | MDS | 4.4 alone | Ongoing | 47 | tbd |

b. Combination Therapy with AP1, Paclitaxel, and Eribulin

The efficacy of AP1 alone and in combination with paclitaxel or eribulin was tested in the MCF-7.1 human breast carcinoma xenograft model using female athymic nude mice. TABLE 15 shows the dosing group numbers and amounts of paclitaxel and eribulin for the combination treatment.

TABLE 15

| | | AP1 | | |
|---|---|---|---|---|
| Drug | Amount | 0 mg/kg | 5 mg/kg | 10 mg/kg |
| Paclitaxel | 0 mg/kg | 1 | 3 | 2 |
| | 10 mg/kg | 5 | 10 | 9 |
| | 15 mg/kg | 4 | 8 | 7 |
| Eribulin | 0.1 mg/kg | 6 | 12 | 11 |

Animals were provided with drinking water with 10 μg/mL of 17 beta-estradiol supplementation, 3 days prior to cell implementation and for the duration of the study. Charles River NCr nu/nu mice were treated with subcutaneous injections of 1×10⁷ MCF-7.1 tumor cells in 0% Matrigel® in the flank. The cell injection volume was 0.1 mL/mouse. The mice were 8-12 weeks of age at the start of the study. Body weight and caliper measurements were made biweekly to the end of the study. Any individual animal with a single observation of >30% body weight loss or three consecutive measurements of >25% body weight loss was euthanized. Any group with a mean body weight loss of >20% or >10% mortality was not given further dosages. The groups were not euthanized and recovery was allowed. Within a group with >20% weight loss, individuals hitting the individual body weight loss endpoint was euthanized. If the group treatment-related body weight loss was recovered within 10% of the original weight, dosing was resumed at a loser dose or less frequent dosing schedule. Exceptions to non-treatment body weight % recovery were allowed on a case-by-case basis. Animals were monitored individually. The endpoint of the experiment was a tumor volume of 1000 mm³ or 60 days, whichever came first. Responders were followed for a longer period of time. When the endpoint was reached, the animals were euthanized.

AP1 was prepared as a phosphate-buffered aqueous solution. Paclitaxel was prepared in 5% ethanol and 5% cremaphor EL® in D5W. The vehicle was a phosphate-buffered aqueous solution. The dosing volume was 10 mL/kg (0.2 mL/20 g mouse). The volume was adjusted accordingly for the body weight of each mouse.

TABLE 16 shows results from the paclitaxel combination therapy efficacy test in MCF-7 subjects using AP1, paclitaxel, and eribulin. After 28 days, the surviving animals were followed to the tumor size endpoint or death.

TABLE 16

| Arm | Treatment A | Treatment B | N | Dosing schedule |
|---|---|---|---|---|
| 1 | Vehicle | Vehicle | 10 | Days 1, 4, 8, 11, 15, 18, 22, 25 |
| 2 | AP1 10 mg/kg | Vehicle | 10 | Days 1, 4, 8, 11, 15, 18, 22, 25 |
| 3 | AP1 5 mg/kg | Vehicle | 10 | Days 1, 4, 8, 11, 15, 18, 22, 25 |
| 4 | Paclitaxel 15 mg/kg | Vehicle | 10 | Days 1, 8, 15, 22 |
| 5 | Paclitaxel 10 mg/kg | Vehicle | 10 | Days 1, 8, 15, 22 |
| 6 | Eribulin 0.1 mg/kg | Vehicle | 10 | Days 1, 8, 15, 22 |
| 7 | AP1 10 mg/kg | Paclitaxel 15 mg/kg | 10 | Days 1, 8, 15, 22: Paclitaxel followed by |
| | | | | AP1 6 h later |
| | | | | Days 4, 11, 18, 25: AP1 only |
| 8 | AP1 5 mg/kg | Paclitaxel 15 mg/kg | 10 | Days 1, 8, 15, 22: Paclitaxel followed by |
| | | | | AP1 6 h later |
| | | | | Days 4, 11, 18, 25: AP1 only |
| 9 | AP1 10 mg/kg | Paclitaxel 10 mg/kg | 10 | Days 1, 8, 15, 22: Paclitaxel followed by |
| | | | | AP1 6 h later |
| | | | | Days 4, 11, 18, 25: AP1 only |
| 10 | AP1 5 mg/kg | Paclitaxel 10 mg/kg | 10 | Days 1, 8, 15, 22: Paclitaxel followed by |
| | | | | AP1 6 h later |
| | | | | Days 4, 11, 18, 25: AP1 only |
| 11 | AP1 10 mg/kg | Eribulin 0.1 mg/kg | 10 | Days 1, 8, 15, 22: Eribulin followed by |
| | | | | AP1 6 h later |
| | | | | Days 4, 11, 18, 25: AP1 only |
| 12 | AP1 5 mg/kg | Eribulin 0.1 mg/kg | 10 | Days 1, 8, 15, 22: Eribulin followed by |
| | | | | AP1 6 h later |
| | | | | Days 4, 11, 18, 25: AP1 only |

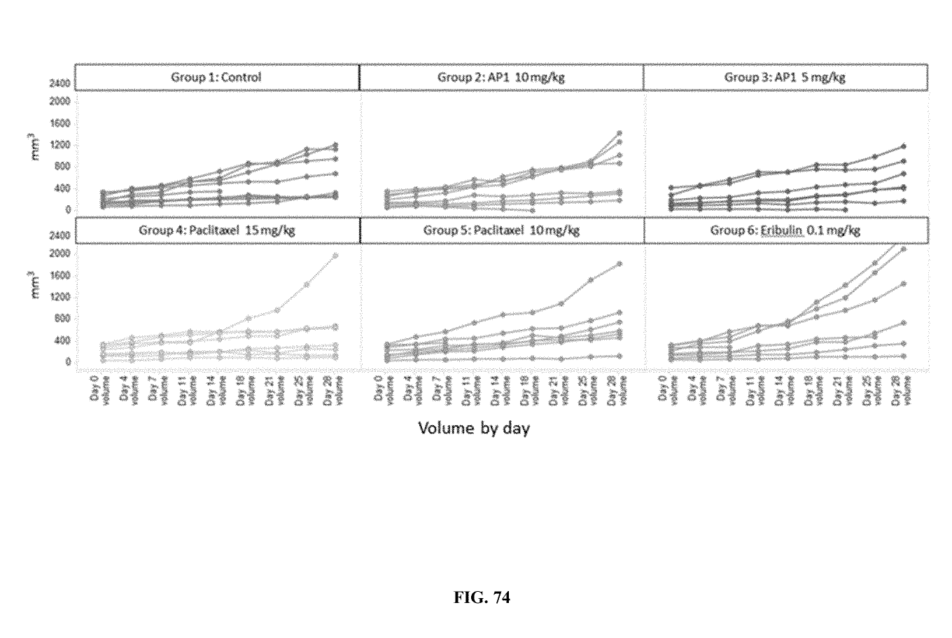

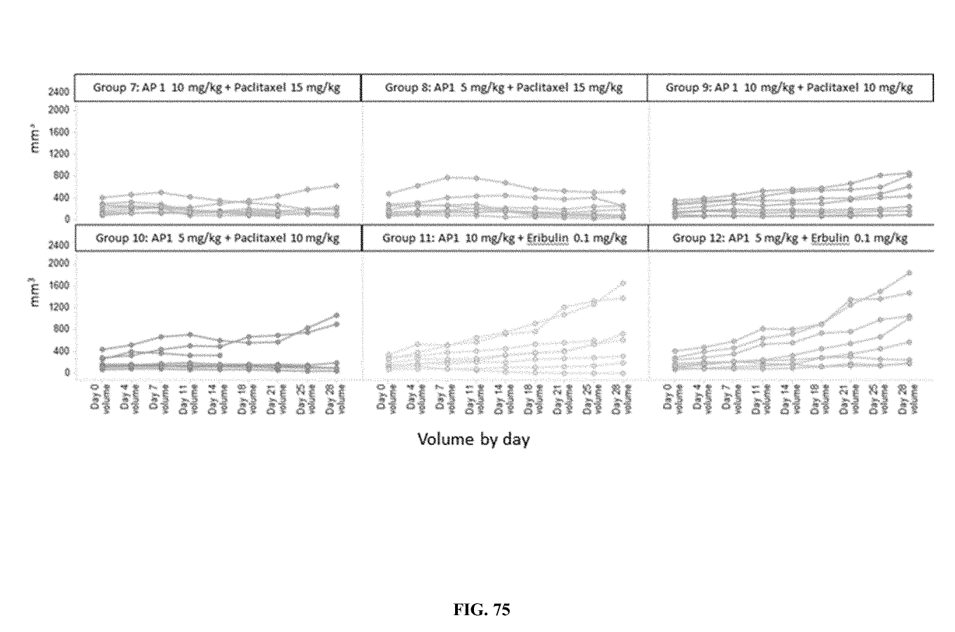

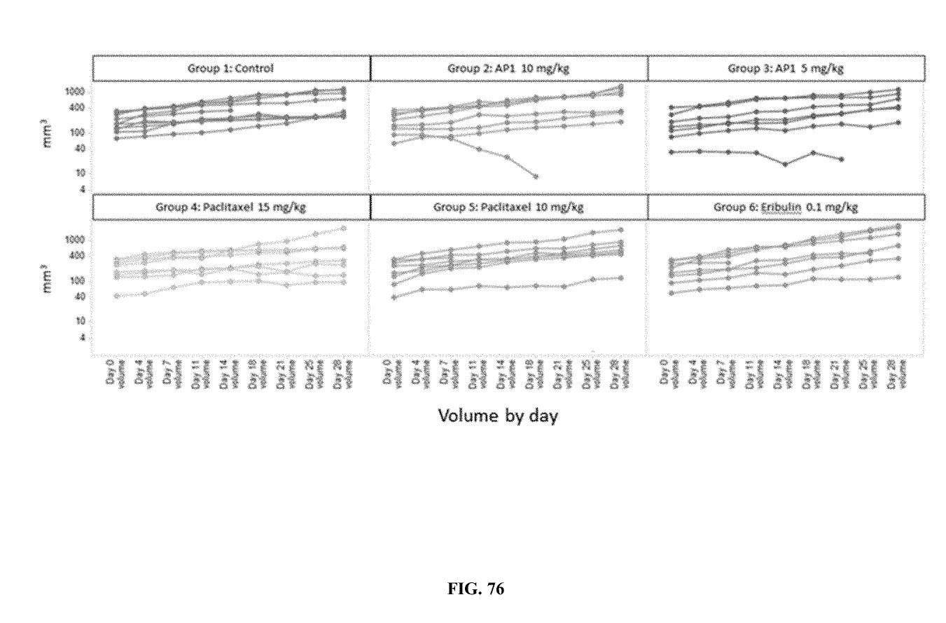

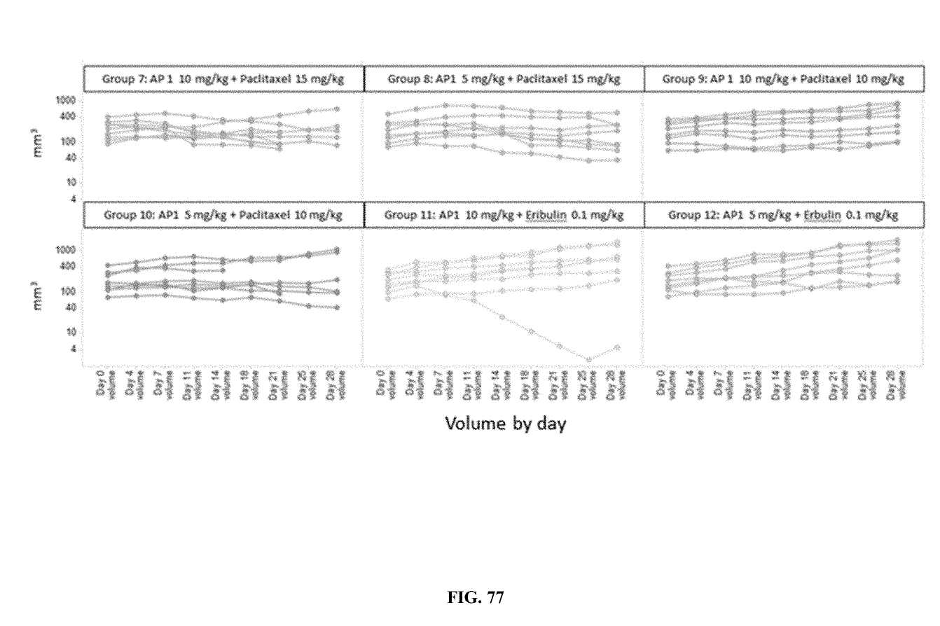

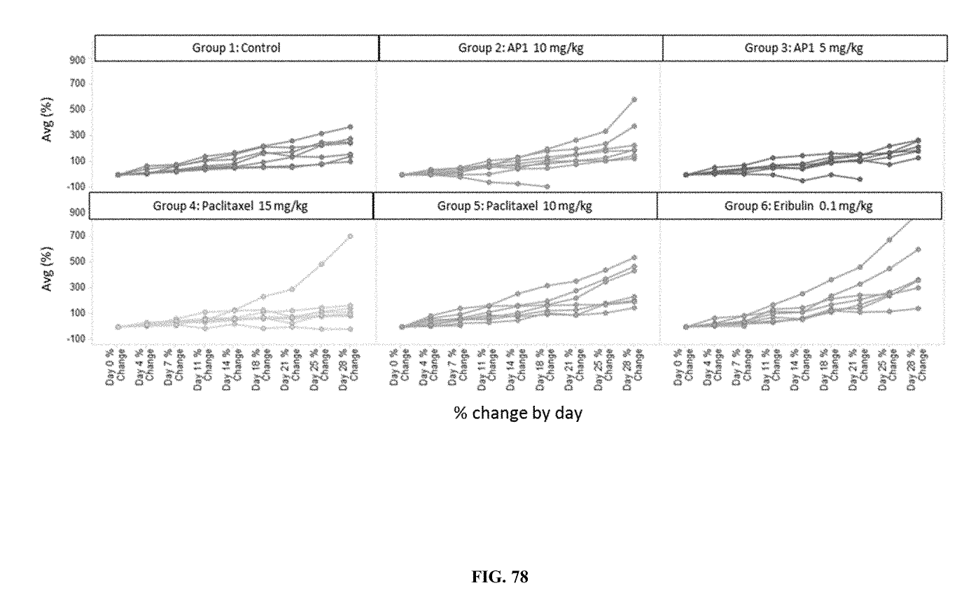

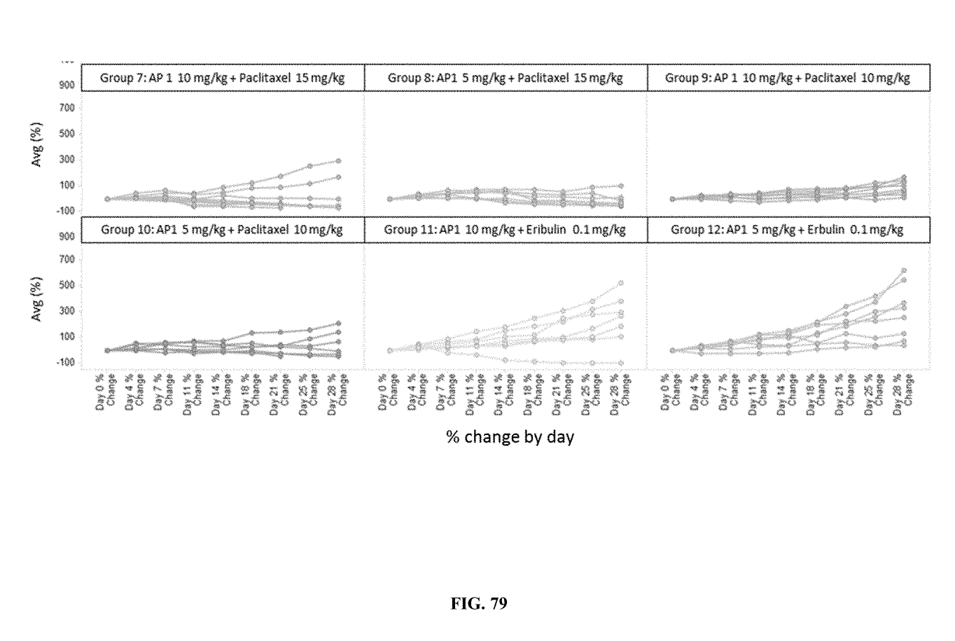

FIG. 74 shows the results of treatment with AP1 or Paclitaxel on individual mouse tumor volume by day. FIG. 75 shows the results of combination treatment with AP1+paclitaxel on individual mouse tumor volume by day. FIG. 76 shows the results of treatment with AP1 or Paclitaxel on individual mouse tumor volume by day on a Log₁₀ axis to show growth. FIG. 77 shows the results of combination treatment with AP1+paclitaxel on individual mouse tumor volume by day on a Log₁₀ axis to show growth. FIG. 78 shows the results of treatment with AP1 or Paclitaxel on individual mouse tumor volume % change from baseline by day. FIG. 79 shows the results of combination treatment with AP1+paclitaxel on individual mouse tumor volume % change from baseline by day.

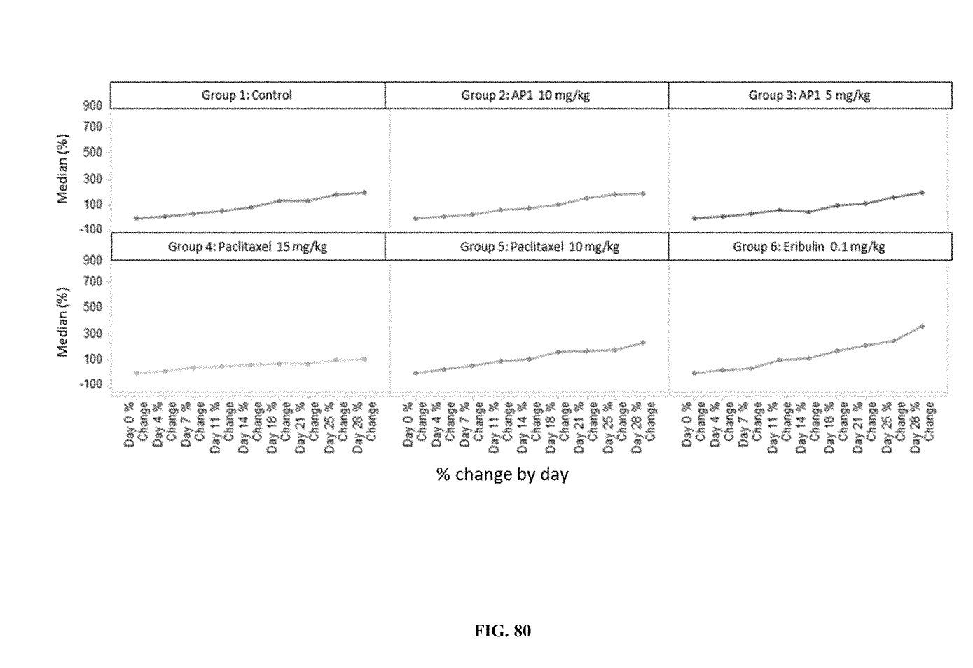

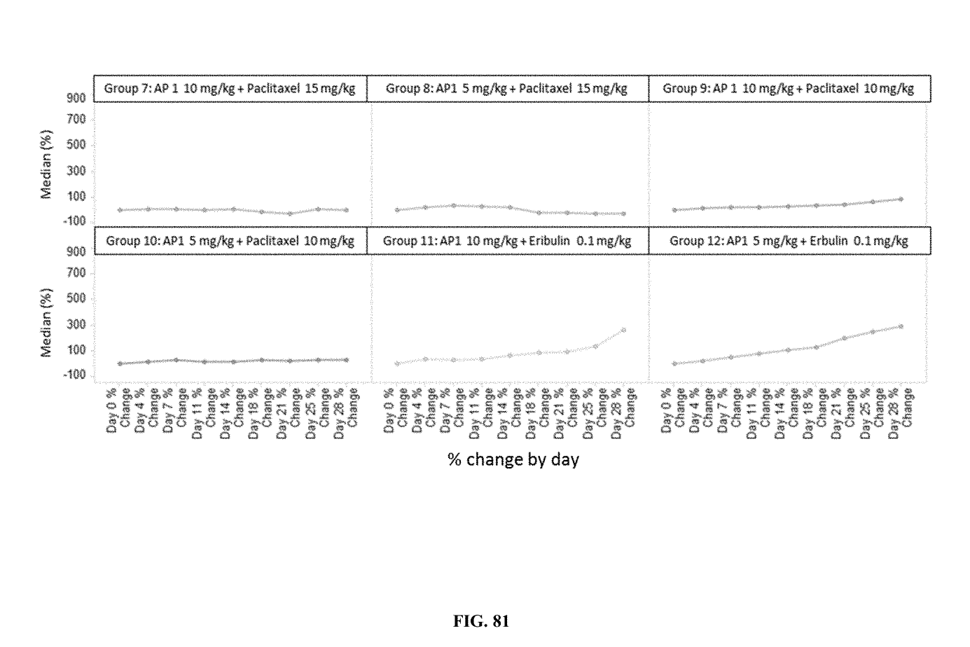

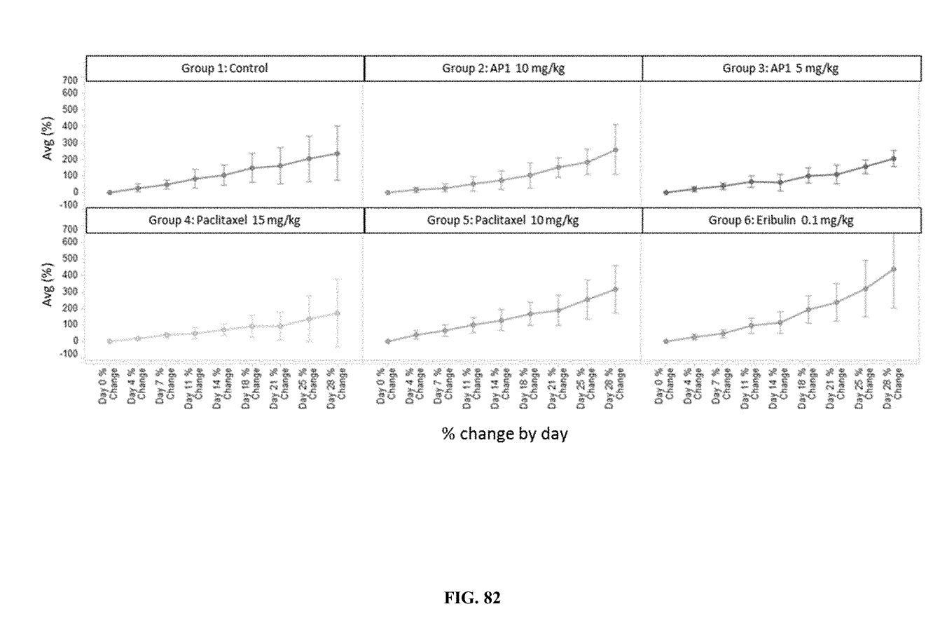

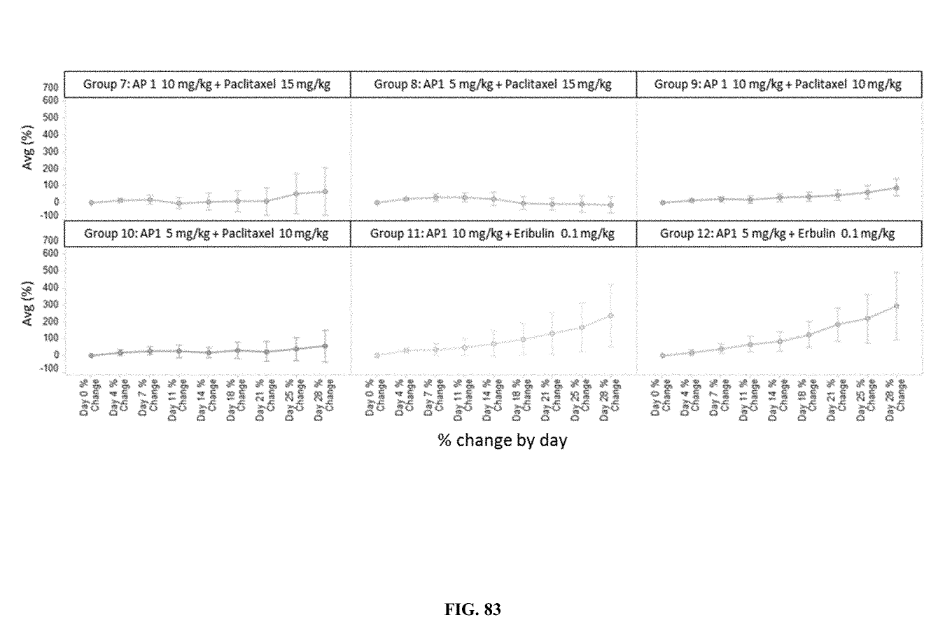

FIG. 80 shows the results of treatment with AP1 or Paclitaxel on median tumor volume % change from baseline by day. FIG. 81 shows the results of combination treatment with AP1+paclitaxel on median tumor volume % change from baseline by day. FIG. 82 shows the results of treatment with AP1 or Paclitaxel on average (±1 StDev) tumor volume % change from baseline by day. FIG. 83 shows the results of combination treatment with AP1+paclitaxel on average (±1 StDev) tumor volume % change from baseline by day.

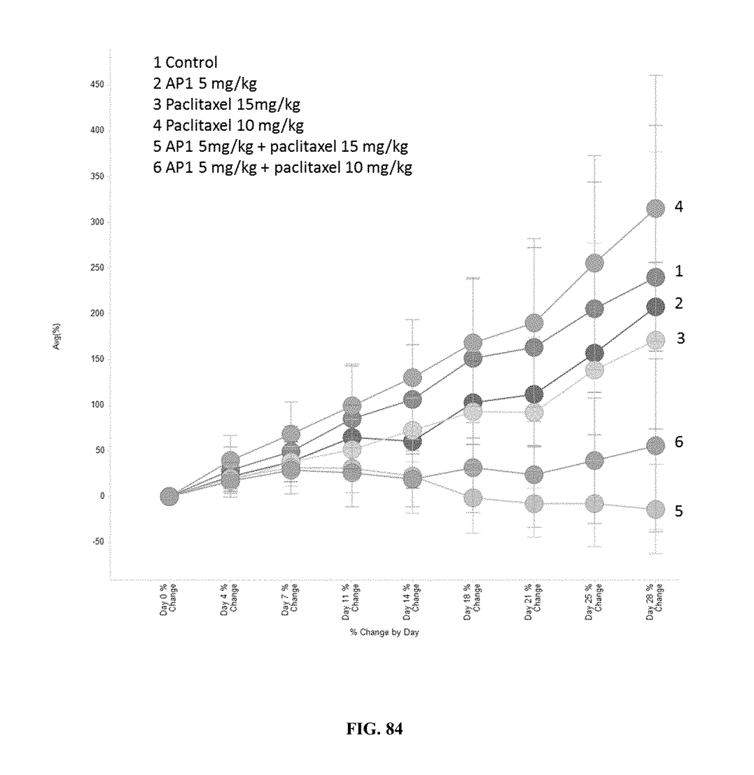

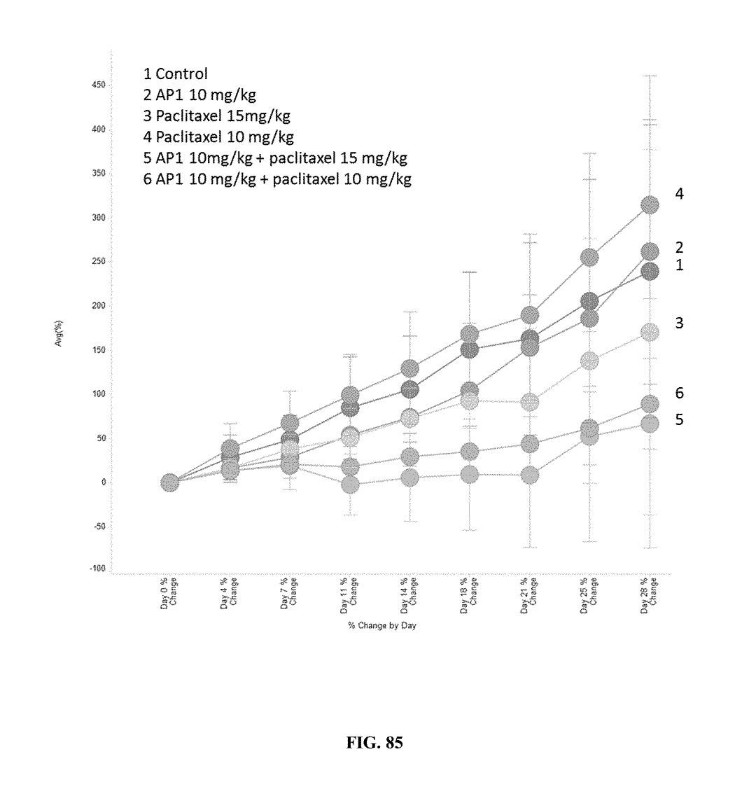

FIG. 84 compares the results of treatment with AP1, paclitaxel, or combination treatment with AP1+paclitaxel on the average % change in tumor volume from baseline per day. The data show that combination therapy with 5 mg/kg AP1 and 10 mg/kg paclitaxel; or 5 mg/kg AP1 and 15 mg/kg paclitaxel minimized the average % change in tumor volume from baseline for the duration of the study. FIG. 85 compares the results of treatment with AP1, paclitaxel, or combination treatment with AP1+paclitaxel on the average % change in tumor volume from baseline per day. The data show that combination therapy with 10 mg/kg AP1 and 10 mg/kg paclitaxel; or 5 mg/kg AP1 and 15 mg/kg paclitaxel minimized the average % change in tumor volume from baseline for the duration of the study.

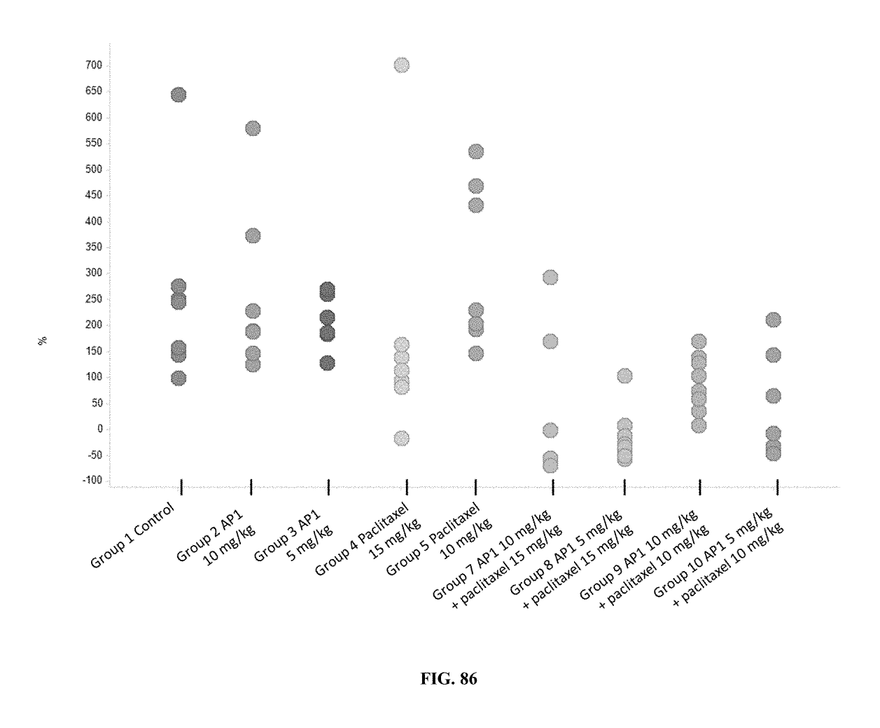

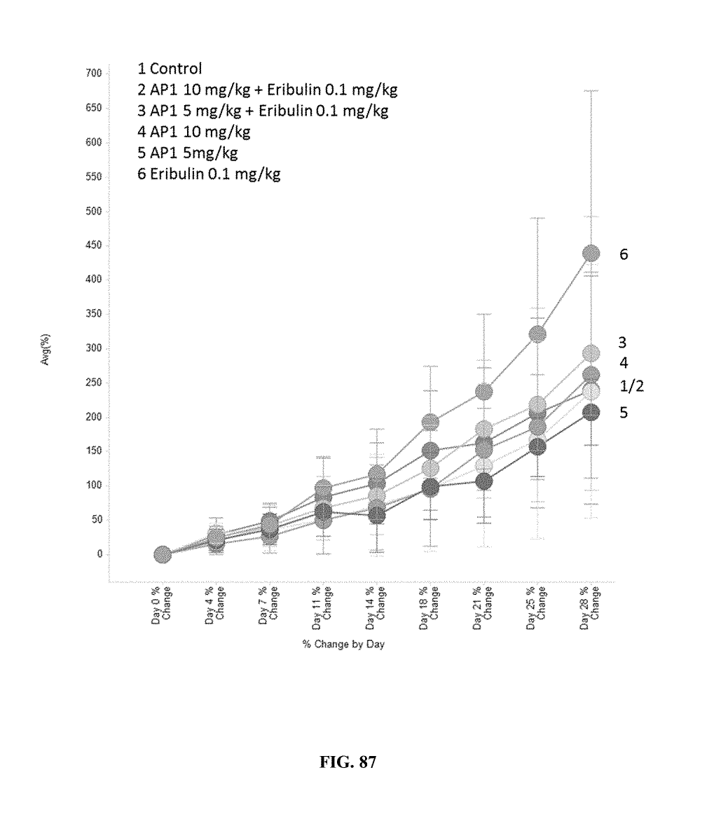

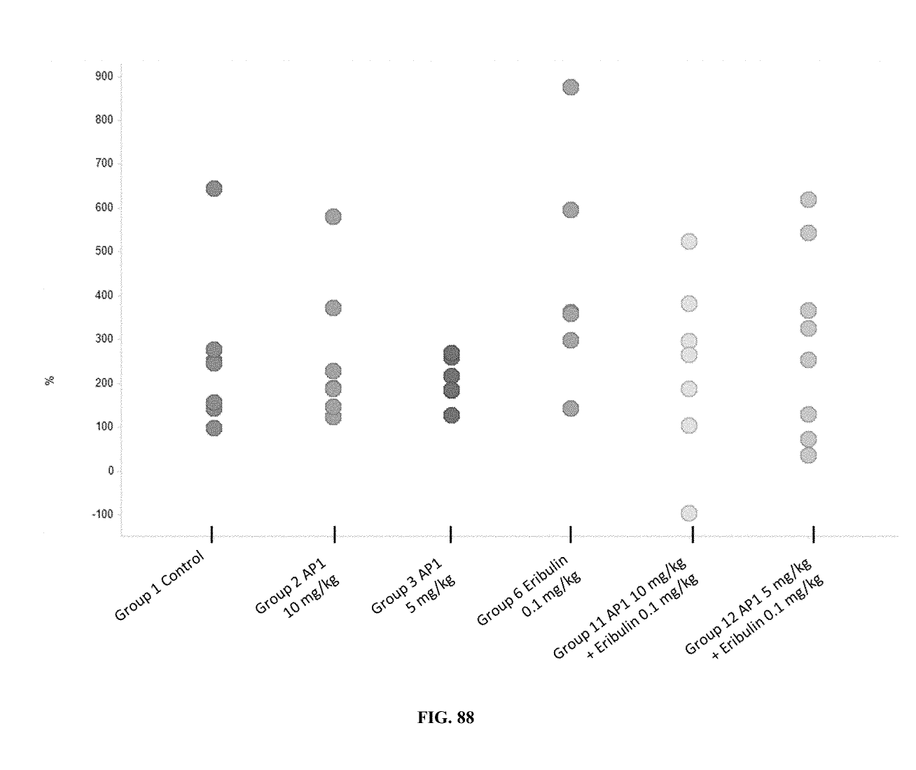

FIG. 86 shows the effect of treatment with AP1, paclitaxel, or combination treatment with AP1+paclitaxel on individual tumor volume % change from baseline on Day 28 per study group. Group 1: control; Group 2: AP110 mg/kg; Group 3: AP1 5 mg/kg; Group 4: paclitaxel 15 mg/kg; Group 5: paclitaxel 10 mg/kg; Group 7: combination treatment AP1 10 mg/kg+paclitaxel 15 mg/kg; Group 8: combination treatment AP1 15 mg/kg+paclitaxel 15 mg/kg; Group 9: combination treatment AP1 10 mg/kg+paclitaxel 10 mg/kg; Group 10: AP1 5 mg/kg+paclitaxel 10 mg/kg. FIG. 87 shows the effect of treatment with AP1, eribulin, or combination treatment with AP1+eribulin on the average % change of tumor volume. Line 1: control; Line 2: combination treatment with AP1 10 mg/kg+eribulin 0.1 mg/kg; Line 3: combination treatment with AP1 5 mg/kg+eribulin 0.1 mg/kg; Line 4: AP1 10 mg/kg; Line 5: AP1 5 mg/kg; Line 6: eribulin 0.1 mg/kg. FIG. 88 shows the effect of treatment with AP1, eribulin, or combination treatment with AP1+eribulin on individual tumor volume? % change from baseline on Day 28. Group 1: control; Group 2: AP1 10 mg/kg; Group 3: AP1 5 mg/kg; Group 6: eribulin 0.1 mg/kg; Group 11: combination treatment with AP1 10 mg/kg+eribulin 0.1 mg/kg; Group 12: combination treatment with AP1 5 mg/kg+eribulin 0.1 mg/kg.

c. Combination Therapy with AP1 and Abraxane®

Abraxane®, also known as protein-bound paclitaxel or nanoparticle albumin-bound paclitaxel, is an injectable formulation of paclitaxel used to treat breast cancer, lung cancer, and pancreatic cancer. The efficacy of AP1 alone and in combination with Abraxane® was tested in the MCF-7.1 human breast carcinoma xenograft model using female athymic nude mice, following the method used to test the efficacy of AP1 in combination with paclitaxel.

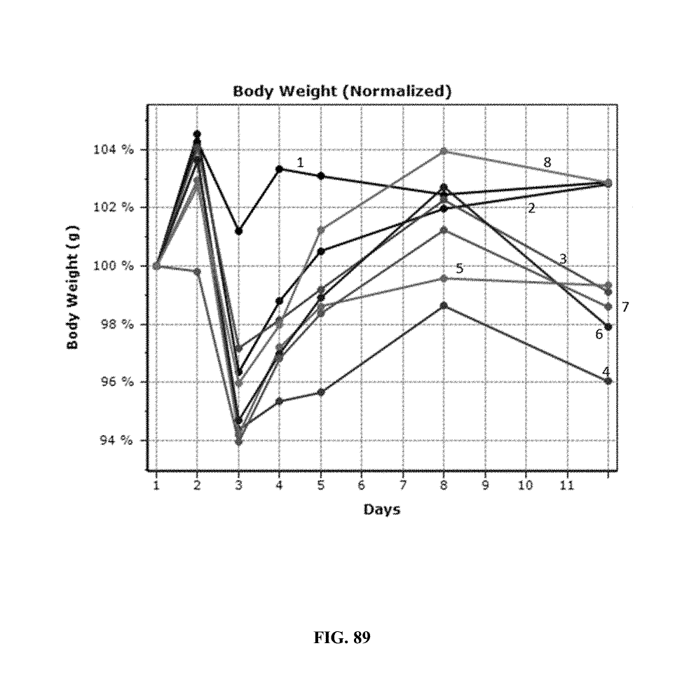

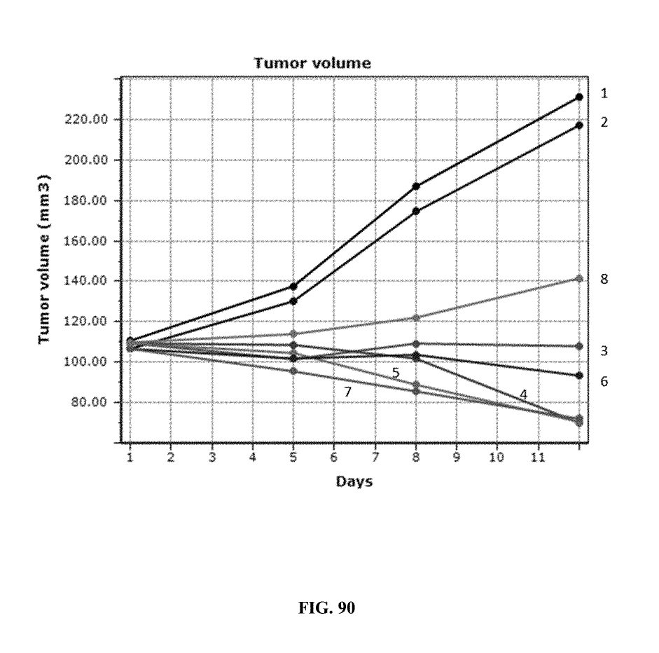

FIG. 89 shows changes in the normalized body weights of mice treated under various dosing regimens of AP1, Abraxane®, or combination treatment with AP1+Abraxane® over a period of 12 days in the MCF-7.1 human breast carcinoma xenograft model. FIG. 90 shows changes in tumor volumes (mm³) of mice treated under various dosing regimens of AP1, Abraxane®, or combination treatment with AP1+Abraxane® over a period of 12 days in the MCF-7.1 human breast carcinoma xenograft model.

TABLE 17 shows the dosing regimens used to obtain data on the efficacy of combination treatment using AP1 and Abraxane®.

TABLE 17

| Group # | Dosing |
|---|---|
| Group 1 | vehicle (i.v., days 2, 5, 9, 12, 16, 19, 23, 26) |
| Group 2 | AP1 5 mg/kg (i.v., days 2, 5, 9, 12, 16, 19, 23, 26) |
| Group 3 | Abraxane ® 15 mg/kg (i.v., qwk × 4 starting on day 2) |
| Group 4 | combination treatment with AP1 5 mg/kg (i.v., days 2, 5, 9, |
| | 12, 16, 19, 23, 26) + Abraxane ® 15 mg/kg |
| | (i.v., qwk × 4 starting on day 4) |
| Group 5 | combination treatment with AP1 5 mg/kg (i.v., days 2, 5, 9, 12, |
| | 16, 19, 23, 26; dose 6 hours prior to Abraxane ®) + |
| | Abraxane ® 15 mg/kg (i.v., qwk × 4 starting on day 2) |
| Group 6 | combination treatment with Abraxane ® 15 mg/kg |
| | (i.v., qwk × 4 starting on day 2) + AP1 5 mg/kg (i.v., days 2, 5, |
| | 9, 12, 16, 19, 23, 26; dose 6 hours post-Abraxane ®) |
| Group 7 | Combination treatment with Abraxane ® 15 mg/kg |
| | (i.v., qwk × 4) + AP1 5 mg/kg (i.v., days 2, 5, 9, 12, 16, 19, |
| | 23, 26; dose 24 hours post-Abraxane ®) |
| Group 8 | Combination treatment with AP1 5 mg/kg (i.v., days 2, 5, 9, 12, |
| | 16, 19, 23, 26; dose 24 hours prior to Abraxane ®) + |
| | Abraxane ® 15 mg/kg (i.v., qwk × 4 starting on day 3) |

The data show that Group 7, Group 6, Group 5, and Group 4 resulted in an overall reduction in tumor volume upon treatment. Group 7 had the highest reduction in tumor volume 5 days after treatment.

### Example 30: Combination Therapy with AP1 and PD-1 or PD-L1 Antagonists

a. Mice Treated with CloudmanS91 Malignant Melanoma Tumors

The efficacy of AP1 in combination with murine anti-PD-1, anti-PD-L1, or anti-CTLA-4 was tested in syngeneic mouse models. The murine syngeneic models used for the studies were CT-26 for CTLA-4; CloudmanS91, Colon26, EMT-6, A20, and MC-38 for PD-1; and CloudmanS91, A20, MC-38, and B16F10 for PD-L1.

AP1 was administered intravenously starting on D1 at dosages of 5 mg/kg, 10 mg/kg, or 20 mg/kg per body weight of each mouse. AP1 was administered 2 times per week for 2 weeks. Anti-PD-1 was administered I.P. on day 3 at a dose of 5 mg/kg, twice a week for two weeks. Anti-PD-L1 was administered I.P. on day 3 at a dose of 5 mg/kg, twice a week for two weeks. Anti-CTLA-4 was administered I.P. on day 3 at a dose of 5 mg/kg, and then at a dose of 2.5 mg/kg on day 6 and day 10. End points were based on tumor volume, body weight, and clinical observations.

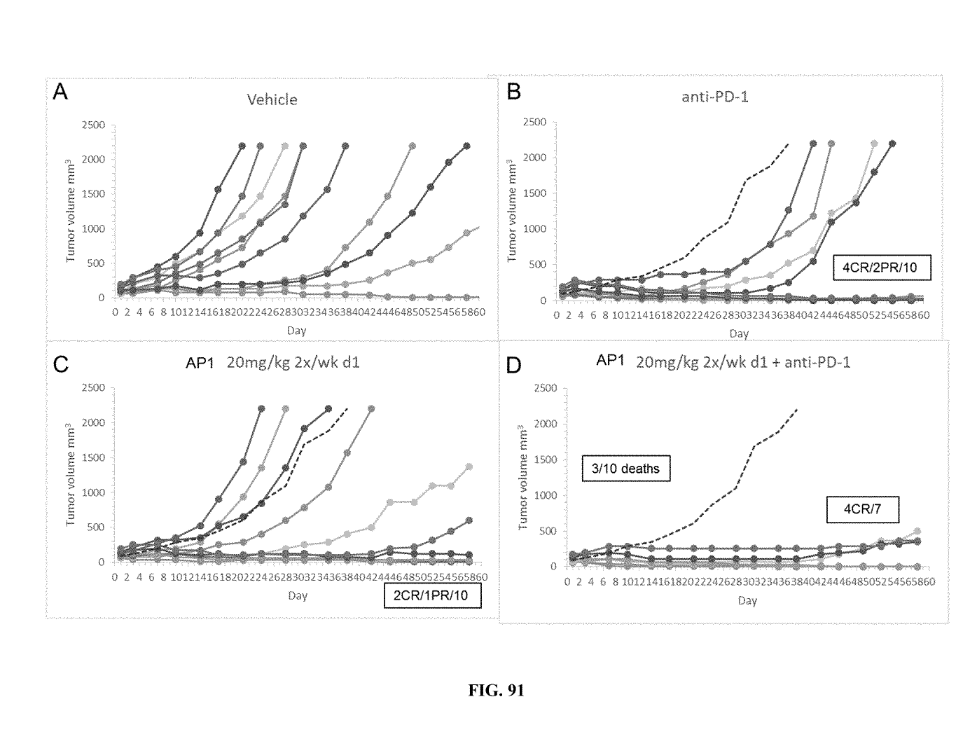

FIG. 91 PANEL A shows the results of vehicle treatment on tumor volumes (mm³) of mice using a CloudmanS91 malignant melanoma model. FIG. 91 PANEL B shows the results of treatment with anti-PD-1 on tumor volumes (mm³) of mice using a CloudmanS91 malignant melanoma model. FIG. 91 PANEL C shows the effect of treatment with twice a week treatment of AP1 at 20 mg/kg on tumor volumes (mm³) of mice using a CloudmanS91 malignant melanoma model. FIG. 91 PANEL D shows the effect of combination treatment with twice a week treatment of AP1 at 20 mg/kg and anti-PD-1 on tumor volumes (mm³) of mice using a CloudmanS91 malignant melanoma model. The dotted line indicates the median tumor volume for the vehicle control.

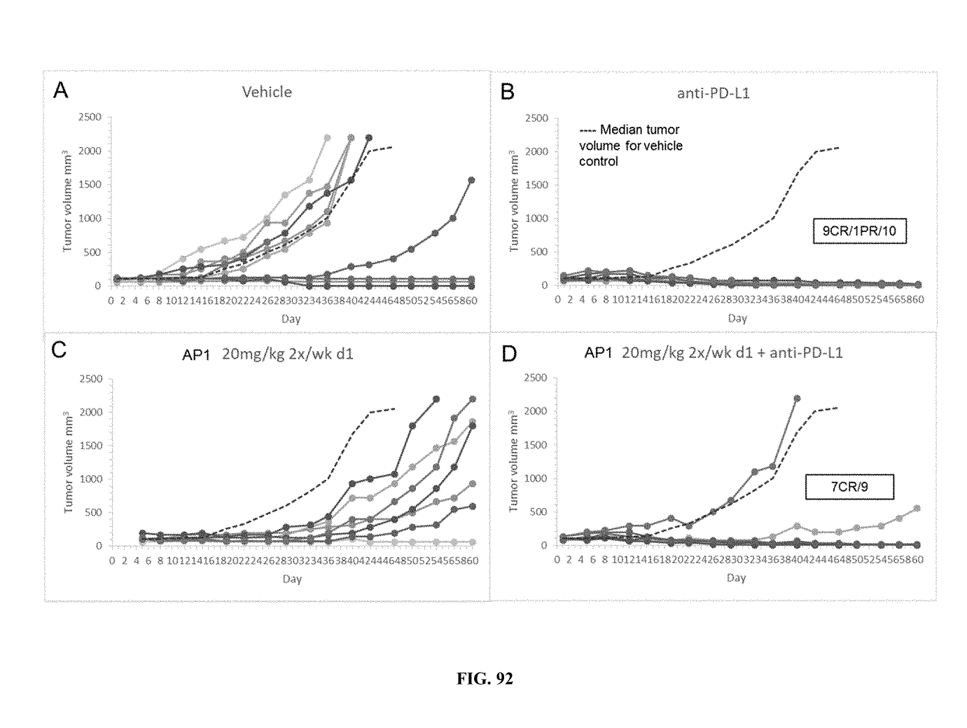

FIG. 92 PANEL A shows the results of vehicle treatment on tumor volumes (mm³) of mice using a CloudmanS91 malignant melanoma model. FIG. 92 PANEL B shows the results of treatment with anti-PD-L1 on tumor volumes (mm³) of mice using a CloudmanS91 malignant melanoma model. FIG. 92 PANEL C shows the effect of treatment with twice a week treatment of AP1 at 20 mg/kg on tumor volumes (mm³) of mice using a CloudmanS91 malignant melanoma model. FIG. 92 PANEL D shows the effect of combination treatment with twice a week treatment of AP1 at 20 mg/kg and anti-PD-L1 on tumor volumes (mm³) of mice using a CloudmanS91 malignant melanoma model. The dotted line indicates the median tumor volume for the vehicle control.

b. Mice Treated with A20 Lymphoma

The efficacy of treatment with AP1 alone and in combination with anti-PD-1 was tested in the A20 murine lymphoma model using female BALB/c mice. Charles River female BALB/c mice were treated subcutaneously in the flank with 1×10⁶ A20 cells in 0% Matrigel®. The cell injection volume was 0.1 mL/mouse. The mice were 8 to 12 weeks of age at the start of the experiment. A pair match was performed when tumors reached an average size of 90-120 mm³, and treatment began. Body weight and caliper measurements were made biweekly throughout the experiment. Dosing volume was 10 mL/kg, and the volume was adjusted accordingly for the body weight of each mouse.

Any individual animal with a single observation of >30% body weight loss or three consecutive measurements of >25% body weight loss was euthanized. Any group with a mean body weight loss of >20% or >10% mortality was not given further dosages. The groups were not euthanized and recovery was allowed. Within a group with >20% weight loss, individuals hitting the individual body weight loss endpoint was euthanized. If the group treatment-related body weight loss was recovered within 10% of the original weight, dosing was resumed at a loser dose or less frequent dosing schedule. Exceptions to non-treatment body weight % recovery were allowed on a case-by-case basis. Animals were monitored individually. The endpoint of the experiment was a tumor volume of 2000 mm³ or 45 days, whichever came first. Responders were followed for a longer period of time. When the endpoint was reached, the animals were euthanized.

Anti-PD-1 RMP1-14 (ratIgG) was used to test the efficacy of combination treatment using AP1 and anti-PD-1. TABLE 18 shows the treatment regimens used to test the efficacy of combination treatment using AP1 and anti-PD-1.

TABLE 18

| | | Regimen 1 | | | | Regimen 2 | | | |
|---|---|---|---|---|---|---|---|---|---|
| Gr. | N | Agent | mg/kg | Route | Schedule | Agent | mg/kg | Route | Schedule |
| 1^{#} | 10 | vehicle | — | iv | biwk x 2 (start | PBS | — | ip | biwk x 2 (start |
| | | | | | on day 1) | | | | on day 3) |
| 2 | 10 | anti-PD-1 | 5 | ip | biwk x 2 (start | — | — | — | — |
| | | RMP1-14 | | | on day 3) | | | | |
| 3 | 10 | AR16 | 20 | iv | biwk x 2 (start | — | — | — | — |
| | | | | | on day 1) | | | | |
| 4 | 10 | AR16 | 20 | iv | biwk x 2 (start | — | — | — | — |
| | | | | | on day 3) | | | | |
| 5 | 10 | AR16 | 20 | iv | biwk x 2 (start | — | — | — | — |
| | | | | | on day 5) | | | | |
| 6 | 10 | AR16 | 5 | iv | biwk x 2 (start | anti-PD-1 | 5 | ip | biwk x 2 (start |
| | | | | | on day 1) | RMP1-14 | | | on day 3) |
| 7 | 10 | AR16 | 10 | iv | biwk x 2 (start | anti-PD-1 | 5 | ip | biwk x 2 (start |
| | | | | | on day 1) | RMP1-14 | | | on day 3) |
| 8 | 10 | AR16 | 20 | iv | biwk x 2 (start | anti-PD-1 | 5 | ip | biwk x 2 (start |
| | | | | | on day 1) | RMP1-14 | | | on day 3) |
| 9 | 10 | AR16 | 5 | iv | biwk x 2 (start | anti-PD-1 | 5 | ip | biwk x 2 (start |
| | | | | | on day 3) | RMP1-14 | | | on day 3) |
| 10 | 10 | AR16 | 10 | iv | biwk x 2 (start | anti-PD-1 | 5 | ip | biwk x 2 (start |
| | | | | | on day 3) | RMP1-14 | | | on day 3) |
| 11 | 10 | AR16 | 20 | iv | biwk x 2 (start | anti-PD-1 | 5 | ip | biwk x 2 (start |
| | | | | | on day 3) | RMP1-14 | | | on day 3) |
| 12 | 10 | AR16 | 5 | iv | biwk x 2 (start | anti-PD-1 | 5 | ip | biwk x 2 (start |
| | | | | | on day 5) | RMP1-14 | | | on day 3) |
| 13 | 10 | AR16 | 10 | iv | biwk x 2 (start | anti-PD-1 | 5 | ip | biwk x 2 (start |
| | | | | | on day 5) | RMP1-14 | | | on day 3) |
| 14 | 10 | AR16 | 20 | iv | biwk x 2 (start | anti-PD-1 | 5 | ip | biwk x 2 (start |
| | | | | | on day 5) | RMP1-14 | | | on day 3) |

^{#}control

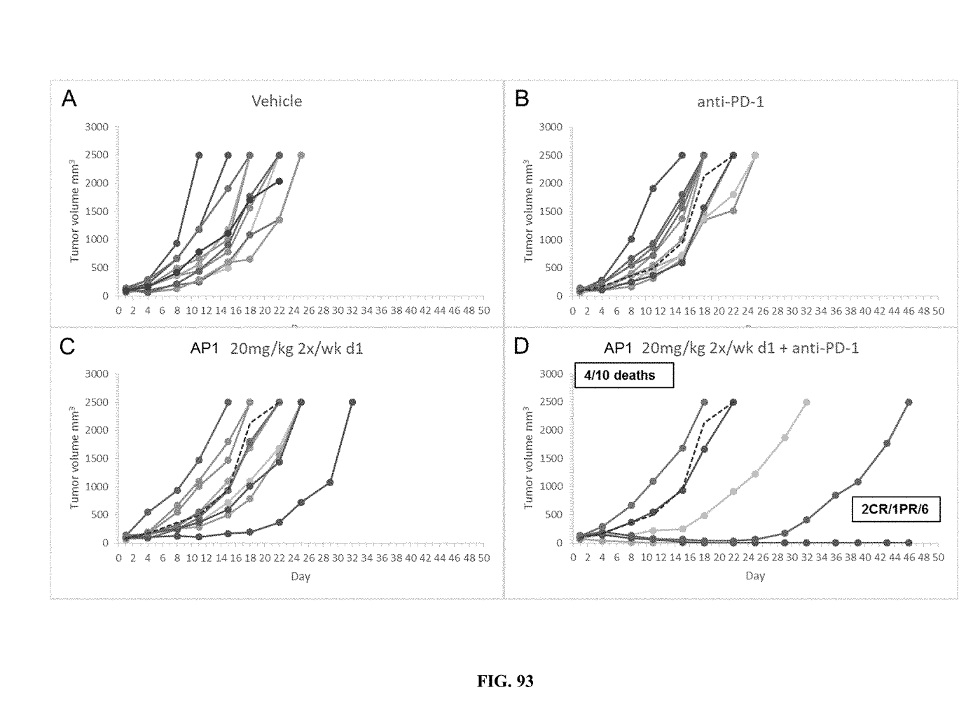

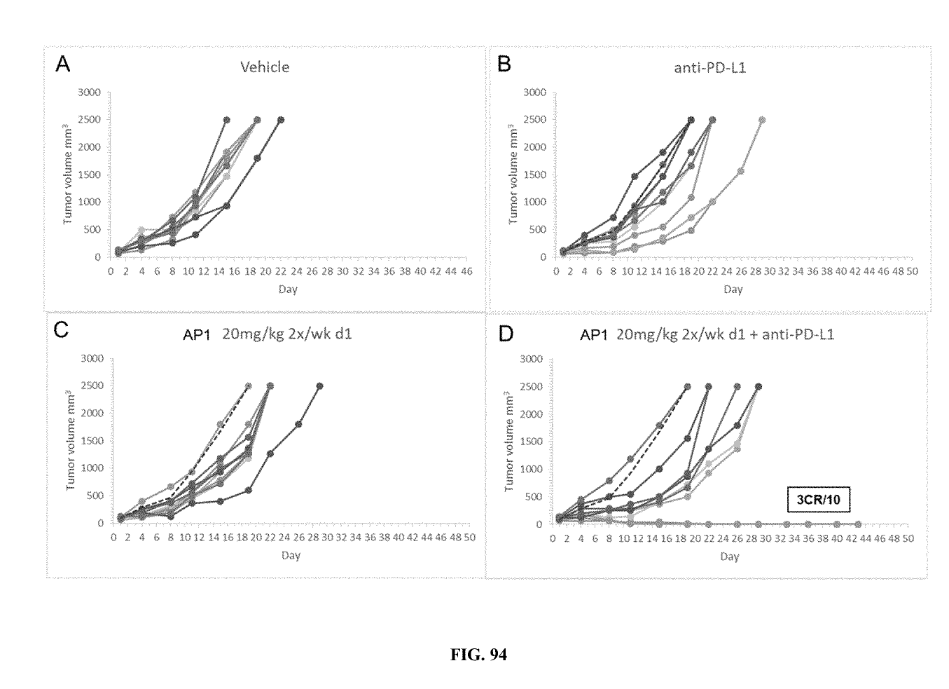

FIG. 93 PANEL A shows the results of vehicle treatment on tumor volumes (mm³) of mice using the A20 murine lymphoma model. FIG. 93 PANEL B shows the results of treatment with anti-PD-1 on tumor volumes (mm³) of mice using the A20 murine lymphoma model. FIG. 93 PANEL C shows the effect of treatment with twice a week treatment of AP1 at 20 mg/kg on tumor volumes (mm³) of mice using the A20 murine lymphoma model. FIG. 93 PANEL D shows the effect of combination treatment with twice a week treatment of AP1 at 20 mg/kg and anti-PD-1 on tumor volumes (mm³) of mice using the A20 murine lymphoma model. The dotted line indicates the median tumor volume for the vehicle control.

Anti-PD-L1 10F.9G2 in PBS was used to test the efficacy of combination treatment using AP1 and anti-PD-L1. The dosing volume for the vehicle and AP1 was 10 mL/kg, and was adjusted accordingly for the body weight of each mouse. The dosing volume for PBS and anti-PD-L1 was 0.2 mL/mouse, and was not adjusted for body weight. TABLE 19 shows the treatment regimens used to test the efficacy of combination treatment using AP1 and anti-PD-L1.

TABLE 19

| | | Regimen 1 | | | | Regimen 2 | | | |
|---|---|---|---|---|---|---|---|---|---|
| Gr. | N | Agent | mg/kg | Route | Schedule | Agent | mg/kg | Route | Schedule |
| 1^{#} | 10 | vehicle | — | iv | biwk x 2 (start | PBS | — | ip | biwk x 2 (start |
| | | | | | on day 1) | | | | on day 3) |
| 2 | 10 | anti-PD-L1 | 100* | ip | biwk x 2 (start | — | — | — | — |
| | | | | | on day 3) | | | | |
| 3 | 10 | AR16 | 20 | iv | biwk x 2 (start | — | — | — | — |
| | | | | | on day 1) | | | | |
| 4 | 10 | AR16 | 20 | iv | biwk x 2 (start | — | — | — | — |
| | | | | | on day 3) | — | — | — | — |
| 5 | 10 | AR16 | 20 | iv | biwk x 2 (start | — | — | — | — |
| | | | | | on day 5) | | | | |
| 6 | 10 | AR16 | 5 | iv | biwk x 2 (start | anti-PDL-1 | 100* | ip | biwk x 2 (start |
| | | | | | on day 1) | | | | on day 3) |
| 7 | 10 | AR16 | 10 | iv | biwk x 2 (start | anti-PDL-1 | 100* | ip | biwk x 2 (start |
| | | | | | on day 1) | | | | on day 3) |
| 8 | 10 | AR16 | 20 | iv | biwk x 2 (start | anti-PDL-1 | 100* | ip | biwk x 2 (start |
| | | | | | on day 1) | | | | on day 3) |
| 9 | 10 | AR16 | 5 | iv | biwk x 2 (start | anti-PDL-1 | 100* | ip | biwk x 2 (start |
| | | | | | on day 3) | | | | on day 3) |
| 10 | 10 | AR16 | 10 | iv | biwk x 2 (start | anti-PDL-1 | 100* | ip | biwk x 2 (start |
| | | | | | on day 3) | | | | on day 3) |
| 11 | 10 | AR16 | 20 | iv | biwk x 2 (start | anti-PDL-1 | 100* | ip | biwk x 2 (start |
| | | | | | on day 3) | | | | on day 3) |
| 12 | 10 | AR16 | 5 | iv | biwk x 2 (start | anti-PDL-1 | 100* | ip | biwk x 2 (start |
| | | | | | on day 5) | | | | on day 3) |
| 13 | 10 | AR16 | 10 | iv | biwk x 2 (start | anti-PDL-1 | 100* | ip | biwk x 2 (start |
| | | | | | on day 5) | | | | on day 3) |
| 14 | 10 | AR16 | 20 | iv | biwk x 2 (start | anti-PDL-1 | 100* | ip | biwk x 2 (start |
| | | | | | on day 5) | | | | on day 3) |

^{#}control
*μg/animal

FIG. 94 PANEL A shows the results of vehicle treatment on tumor volumes (mm³) of mice using the A20 murine lymphoma model. FIG. 94 PANEL B shows the results of treatment with anti-PD-L1 on tumor volumes (mm³) of mice using the A20 murine lymphoma model. FIG. 94 PANEL C shows the effect of treatment with twice a week treatment of AP1 at 20 mg/kg on tumor volumes (mm³) of mice using the A20 murine lymphoma model. FIG. 94 PANEL D shows the effect of combination treatment with twice a week treatment of AP1 at 20 mg/kg and anti-PD-L1 on tumor volumes (mm³) of mice using the A20 murine lymphoma model. The dotted line indicates the median tumor volume for the vehicle control.

c. Mice Treated with M38 Syngeneic Colon Carcinoma

The efficacy of AP1 alone and in combination with anti-PD-1 and anti-PD-L1 was tested in the M38 syngeneic colon carcinoma model using C57BL/6 female mice.

Mice were anesthetized with isoflurane for the implantation of cells to reduce ulcerations. Charles River female C57BL/6 mice were treated subcutaneously in the flank with 5×10⁵ MC38 tumor cells in 0% Matrigel®. The cell injection volume was 0.1 mL/mouse. The mice were 8-12 weeks of age at the beginning of the experiments. A pair match was performed when tumors reached an average size of 80-120 mm³. Body weight and caliper measurements were made biweekly throughout the duration of the experiment.

Any individual animal with a single observation of >30% body weight loss or three consecutive measurements of >25% body weight loss was euthanized. Any group with a mean body weight loss of >20% or >10% mortality was not given further dosages. The groups were not euthanized and recovery was allowed. Within a group with >20% weight loss, individuals hitting the individual body weight loss endpoint was euthanized. If the group treatment-related body weight loss was recovered within 10% of the original weight, dosing was resumed at a loser dose or less frequent dosing schedule. Exceptions to non-treatment body weight % recovery were allowed on a case-by-case basis. Animals were monitored individually. The endpoint of the experiment was a tumor volume of 1000 mm³ or 45 days, whichever came first. Responders were followed for a longer period of time. When the endpoint was reached, the animals were euthanized.

Anti-PD-1 RMP1-14 (ratIgG) was used to test the efficacy of combination treatment using AP1 and anti-PD-1. TABLE 20 shows the treatment regimens used to test the efficacy of combination treatment using AP1 and anti-PD-1.

TABLE 20

| | | Regimen 1 | | | | Regimen 2 | | | |
|---|---|---|---|---|---|---|---|---|---|
| Gr. | N | Agent | mg/kg | Route | Schedule | Agent | mg/kg | Route | Schedule |
| 1^{#} | 10 | vehicle | — | iv | biwk x 2 (start | PBS | — | ip | biwk x 2 (start |
| | | | | | on day 1) | | | | on day 3) |
| 2 | 10 | anti-PD-1 | 5 | ip | biwk x 2 (start | — | — | — | — |
| | | RMP1-14 | | | on day 3) | | | | |
| 3 | 10 | AR16 | 20 | iv | biwk x 2 (start | — | — | — | — |
| | | | | | on day 1) | | | | |
| 4 | 10 | AR16 | 20 | iv | biwk x 2 (start | — | — | — | — |
| | | | | | on day 3) | | | | |
| 5 | 10 | AR16 | 20 | iv | biwk x 2 (start | — | — | — | — |
| | | | | | on day 5) | | | | |
| 6 | 10 | AR16 | 5 | iv | biwk x 2 (start | anti-PD-1 | 5 | ip | biwk x 2 (start |
| | | | | | on day 1) | RMP1-14 | | | on day 3) |
| 7 | 10 | AR16 | 10 | iv | biwk x 2 (start | anti-PD-1 | 5 | ip | biwk x 2 (start |
| | | | | | on day 1) | RMP1-14 | | | on day 3) |
| 8 | 10 | AR16 | 20 | iv | biwk x 2 (start | anti-PD-1 | 5 | ip | biwk x 2 (start |
| | | | | | on day 1) | RMP1-14 | | | on day 3) |
| 9 | 10 | AR16 | 5 | iv | biwk x 2 (start | anti-PD-1 | 5 | ip | biwk x 2 (start |
| | | | | | on day 3) | RMP1-14 | | | on day 3) |
| 10 | 10 | AR16 | 10 | iv | biwk x 2 (start | anti-PD-1 | 5 | ip | biwk x 2 (start |
| | | | | | on day 3) | RMP1-14 | | | on day 3) |
| 11 | 10 | AR16 | 20 | iv | biwk x 2 (start | anti-PD-1 | 5 | ip | biwk x 2 (start |
| | | | | | on day 3) | RMP1-14 | | | on day 3) |
| 12 | 10 | AR16 | 5 | iv | biwk x 2 (start | anti-PD-1 | 5 | ip | biwk x 2 (start |
| | | | | | on day 5) | RMP1-14 | | | on day 3) |
| 13 | 10 | AR16 | 10 | iv | biwk x 2 (start | anti-PD-1 | 5 | ip | biwk x 2 (start |
| | | | | | on day 5) | RMP1-14 | | | on day 3) |
| 14 | 10 | AR16 | 20 | iv | biwk x 2 (start | anti-PD-1 | 5 | ip | biwk x 2 (start |
| | | | | | on day 5) | RMP1-14 | | | on day 3) |

^{#}Control group

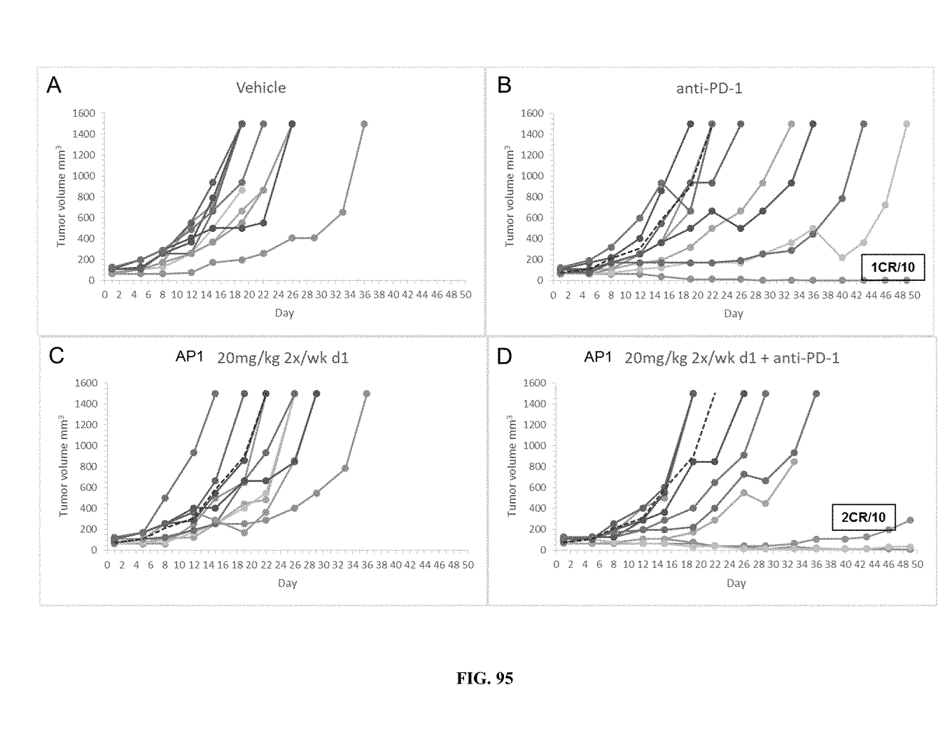

FIG. 95 PANEL A shows the results of vehicle treatment on tumor volumes (mm³) of mice using the M38 syngeneic colon carcinoma model. FIG. 95 PANEL B shows the results of treatment with anti-PD-L1 on tumor volumes (mm³) of mice using the M38 syngeneic colon carcinoma model. FIG. 95 PANEL C shows the effect of treatment with twice a week treatment of AP1 at 20 mg/kg on tumor volumes (mm³) of mice using the M38 syngeneic colon carcinoma model. FIG. 95 PANEL D shows the effect of combination treatment with twice a week treatment of AP1 at 20 mg/kg and anti-PD-L1 on tumor volumes (mm³) of mice using the M38 syngeneic colon carcinoma model. The dotted line indicates the median tumor volume for the vehicle control.

Anti-PD-L1 10F.9G2 in PBS was used to test the efficacy of combination treatment using AP1 and anti-PD-L1. The dosing volume for the vehicle and AP1 was 10 mL/kg, and was adjusted accordingly for the body weight of each mouse. The dosing volume for PBS and anti-PD-L1 was 0.2 mL/mouse, and was not adjusted for body weight. TABLE 21 shows the treatment regimens used to test the efficacy of combination treatment using AP1 and anti-PD-L1.

TABLE 21

| | | Regimen 1 | | | | Regimen 2 | | | |
|---|---|---|---|---|---|---|---|---|---|
| Gr. | N | Agent | mg/kg | Route | Schedule | Agent | mg/kg | Route | Schedule |
| 1^{#} | 10 | vehicle | — | iv | biwk x 2 (start | PBS | — | ip | biwk x 2 (start |
| | | | | | on day 1) | | | | on day 3) |
| 2 | 10 | anti-PDL-1 | 100* | ip | biwk x 2 (start | — | — | — | — |
| | | | | | on day 3) | | | | |
| 3 | 10 | AR16 | 20 | iv | biwk x 2 (start | — | — | — | — |
| | | | | | on day 1) | | | | |
| 4 | 10 | AR16 | 20 | iv | biwk x 2 (start | — | — | — | — |
| | | | | | on day 3) | | | | |
| 5 | 10 | AR16 | 20 | iv | biwk x 2 (start | — | — | — | — |
| | | | | | on day 5) | | | | |
| 6 | 10 | AR16 | 5 | iv | biwk x 2 (start | anti-PDL-1 | 100* | ip | biwk x 2 (start |
| | | | | | on day 1) | | | | on day 3) |
| 7 | 10 | AR16 | 10 | iv | biwk x 2 (start | anti-PDL-1 | 100* | ip | biwk x 2 (start |
| | | | | | on day 1) | | | | on day 3) |
| 8 | 10 | AR16 | 20 | iv | biwk x 2 (start | anti-PDL-1 | 100* | ip | biwk x 2 (start |
| | | | | | on day 1) | | | | on day 3) |
| 9 | 10 | AR16 | 5 | iv | biwk x 2 (start | anti-PDL-1 | 100* | ip | biwk x 2 (start |
| | | | | | on day 3) | | | | on day 3) |
| 10 | 10 | AR16 | 10 | iv | biwk x 2 (start | anti-PDL-1 | 100* | ip | biwk x 2 (start |
| | | | | | on day 3) | | | | on day 3) |
| 11 | 10 | AR16 | 20 | iv | biwk x 2 (start | anti-PDL-1 | 100* | ip | biwk x 2 (start |
| | | | | | on day 3) | | | | on day 3) |
| 12 | 10 | AR16 | 5 | iv | biwk x 2 (start | anti-PDL-1 | 100* | ip | biwk x 2 (start |
| | | | | | on day 5) | | | | on day 3) |
| 13 | 10 | AR16 | 10 | iv | biwk x 2 (start | anti-PDL-1 | 100* | ip | biwk x 2 (start |
| | | | | | on day 5) | | | | on day 3) |
| 14 | 10 | AR16 | 20 | iv | biwk x 2 (start | anti-PDL-1 | 100* | ip | biwk x 2 (start |
| | | | | | on day 5) | | | | on day 3) |

^{#}Control group
*μg/animal

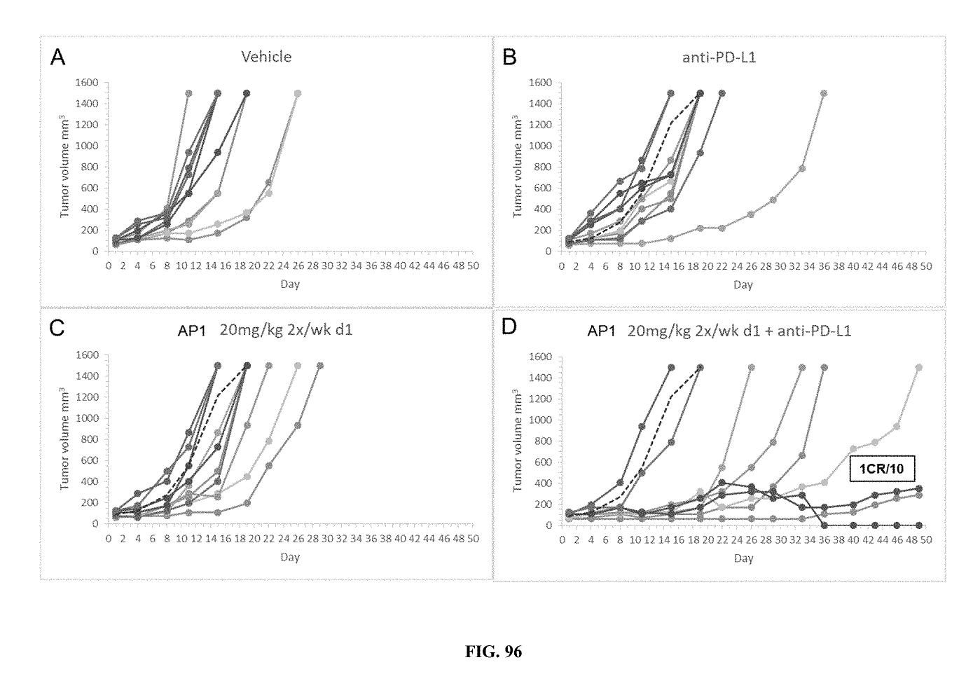

FIG. 96 PANEL A shows the results of vehicle treatment on tumor volumes (mm³) of mice using the M38 syngeneic colon carcinoma model. FIG. 96 PANEL B shows the results of treatment with anti-PD-L1 on tumor volumes (mm³) of mice using the M38 syngeneic colon carcinoma model. FIG. 96 PANEL C shows the effect of treatment with twice a week treatment of AP1 at 20 mg/kg on tumor volumes (mm³) of mice using the M38 syngeneic colon carcinoma model. FIG. 96 PANEL D shows the effect of combination treatment with twice a week treatment of AP1 at 20 mg/kg and anti-PD-L1 on tumor volumes (mm³) of mice using the M38 syngeneic colon carcinoma model. The dotted line indicates the median tumor volume for the vehicle control.

d. Mice Treated with CT26 Undifferentiated Colon Carcinoma Cell Line

The efficacy of AP1 alone and in combination with anti-CTLA-4 was tested in the CT26 undifferentiated colon carcinoma cell line in mice.

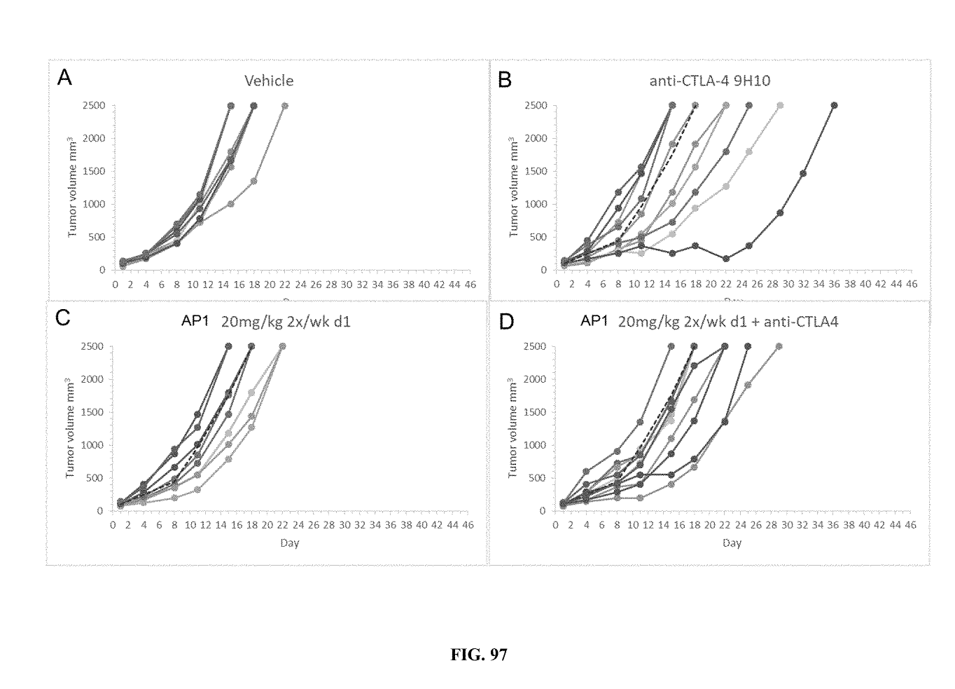

FIG. 97 PANEL A shows the results of vehicle treatment on tumor volumes (mm³) of mice using the CT26 undifferentiated colon carcinoma cell line. FIG. 97 PANEL B shows the results of treatment with anti-CTLA-4 9H10 on tumor volumes (mm³) of mice using the CT26 undifferentiated colon carcinoma cell line. FIG. 97 PANEL C shows the effect of treatment with twice a week treatment of AP1 at 20 mg/kg on tumor volumes (mm³) of mice using the CT26 undifferentiated colon carcinoma cell line. FIG. 97 PANEL D shows the effect of combination treatment with twice a week treatment of AP1 at 20 mg/kg and anti-CTLA-4 on tumor volumes (mm³) of mice using the CT26 undifferentiated colon carcinoma cell line. The dotted line indicates the median tumor volume for the vehicle control.

### EMBODIMENTS

The following non-limiting embodiments provide illustrative examples of the invention, but do not limit the scope of the invention.

### Embodiment 1

A method of treating cancer in a subject in need thereof, the method comprising administering to the subject a therapeutically effective amount of a peptidomimetic macrocycle and at least one additional pharmaceutically active agent, wherein the at least one additional pharmaceutically active agent:
(a) is selected from the group consisting of cobimetinib and binimetinib, or
(b) is a cyclin dependent kinase inhibitor (CDKI) and the CDKI and the peptidomimetic macrocycle are administered with a time separation of more than about 61 minutes;
   wherein the peptidomimetic macrocycle has a Formula:

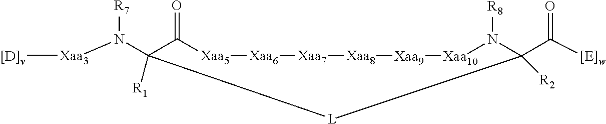

wherein:
each of Xaa₃, Xaa₅, Xaa₆, Xaa₇, Xaa₈, Xaa₉, and Xaa₁₀ is individually an amino acid, wherein at least three of Xaa₃, Xaa₅, Xaa₆, Xaa₇, Xaa₈, Xaa₉, and Xaa₁₀ are the same amino acid as the amino acid at the corresponding position of the sequence Phe₃-X₄-His₅-Tyr₆-Trp₇-Ala₈-Gln₉-Leu₁₀-X₁₁-Ser₁₂ (SEQ ID NO: 8), wherein each X is an amino acid;
each D and E is independently an amino acid;
R₁ and R₂ are independently —H, alkyl, alkenyl, alkynyl, arylalkyl, cycloalkyl, cycloalkylalkyl, heteroalkyl, or heterocycloalkyl, unsubstituted or substituted with halo-; or at least one of R₁ and R₂ forms a macrocycle-forming linker L′ connected to the alpha position of one of said D or E amino acids;
each L and L′ is independently a macrocycle-forming linker of the formula -L₁-L₂-;
each L₁ and L₂ is independently alkylene, alkenylene, alkynylene, heteroalkylene, cycloalkylene, heterocycloalkylene, arylene, heteroarylene, or [—R₄—K—R₄-]ₙ, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
R₇ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
R₈ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
v is an integer from 1-1000;
w is an integer from 3-1000; and
n is an integer from 1-5.

### Embodiment 2

A method of treating cancer in a subject in need thereof, the method comprising administering to the subject a therapeutically effective amount of a peptidomimetic macrocycle and at least one additional pharmaceutically active, wherein the at least one additional pharmaceutically active agent:
(a) is selected from the group consisting of cobimetinib and binimetinib, or
(b) is a cyclin dependent kinase inhibitor (CDKI) and the CDKI and the peptidomimetic macrocycle are administered with a time separation of more than about 61 minutes;
   wherein the peptidomimetic macrocycle has a Formula:

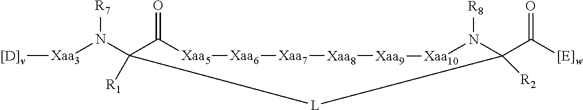

wherein:
each of Xaa₃, Xaa₅, Xaa₆, Xaa₇, Xaa₈, Xaa₉, and Xaa₁₀ is individually an amino acid, wherein at least three of Xaa₃, Xaa₅, Xaa₆, Xaa₇, Xaa₈, Xaa₉, and Xaa₁₀ are the same amino acid as the amino acid at the corresponding position of the sequence Phe₃-X₄-Glu₅-Tyr₆-Trp₇-Ala₈-Gln₉-Leu₁₀/Cba₁₀-X₁₁-Ala₁₂ (SEQ ID NO: 9), wherein each X is an amino acid;
each D and E is independently an amino acid;
R₁ and R₂ are independently —H, alkyl, alkenyl, alkynyl, arylalkyl, cycloalkyl, cycloalkylalkyl, heteroalkyl, or heterocycloalkyl, unsubstituted or substituted with halo-; or at least one of R₁ and R₂ forms a macrocycle-forming linker L′ connected to the alpha position of one of said D or E amino acids;
each L and L′ is independently a macrocycle-forming linker of the formula -L₁-L₂-;
each L₁ and L₂ is independently alkylene, alkenylene, alkynylene, heteroalkylene, cycloalkylene, heterocycloalkylene, arylene, heteroarylene, or [—R₄—K—R₄-]ₙ, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
R₇ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
R₈ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
v is an integer from 1-1000;
w is an integer from 3-1000; and
n is an integer from 1-5.

### Embodiment 3

The method of embodiments 1 or 2, wherein the peptidomimetic macrocycle has improved binding affinity to MDM2 or MDMX relative to a corresponding peptidomimetic macrocycle wherein w is 0, 1 or 2.

### Embodiment 4

The method of embodiments 1 or 2, wherein the peptidomimetic macrocycle has a reduced ratio of binding affinities to MDMX versus MDM2 relative to a corresponding peptidomimetic macrocycle wherein w is 0, 1, or 2.

### Embodiment 5

The method of any one of embodiment 1-4, wherein the peptidomimetic macrocycle has improved in vitro anti-tumor efficacy against p53 positive tumor cell lines relative to a corresponding peptidomimetic macrocycle wherein w is 0, 1, or 2.

### Embodiment 6

The method of any one of embodiments 1-5, wherein the peptidomimetic macrocycle shows improved in vitro induction of apoptosis in p53 positive tumor cell lines relative to a corresponding peptidomimetic macrocycle wherein w is 0, 1, or 2.

### Embodiment 7

The method of any one of embodiments 1-6, wherein the peptidomimetic macrocycle has an improved in vitro anti-tumor efficacy ratio for p53 positive versus p53 negative or mutant tumor cell lines relative to a corresponding peptidomimetic macrocycle wherein w is 0, 1, or 2.

### Embodiment 8

The method of any one of embodiments 1-6, wherein the peptidomimetic macrocycle has improved in vivo anti-tumor efficacy against p53 positive tumors relative to a corresponding peptidomimetic macrocycle wherein w is 0, 1, or 2.

### Embodiment 9

The method of any one of embodiments 1-8, wherein the peptidomimetic macrocycle has improved cell permeability relative to a corresponding peptidomimetic macrocycle wherein w is 0, 1, or 2.

### Embodiment 10

The method of any one of embodiments 1-9, wherein the peptidomimetic macrocycle has improved solubility relative to a corresponding peptidomimetic macrocycle wherein w is 0, 1, or 2.

### Embodiment 11

The method of any one of embodiments 1-10, wherein Xaa₅ is Glu or an amino acid analogue thereof.

### Embodiment 12

The method of any one of embodiments 1-11, wherein Xaa₅ is Glu or an amino acid analogue thereof and wherein the peptidomimetic macrocycle has an improved binding affinity, improved solubility, improved cellular efficacy, improved helicity, improved cell permeability, improved in vivo or in vitro anti-tumor efficacy, or improved induction of apoptosis relative to a corresponding peptidomimetic macrocycle wherein Xaa₅ is Ala.

Embodiment 13 The method of any one of embodiments 1-12, wherein each E is independently an amino acid selected from Ala (alanine), D-Ala (D-alanine), Aib (α-aminoisobutyric acid), Sar (N-methyl glycine), and Ser (serine).

### Embodiment 14

The method of any one of embodiments 1-13, wherein [D]ᵥ is -Leu₁-Thr₂.

### Embodiment 15

The method of any one of embodiments 1-14, wherein w is 3-10.

### Embodiment 16

The method of any one of embodiments 1-15, wherein w is 3-6.

### Embodiment 17

The method of any one of embodiments 1-15, wherein w is 6-10.

### Embodiment 18

The method of any one of embodiments 1-17, wherein w is 6.

### Embodiment 19

The method of any one of any one of embodiments 1-18, wherein v is 1-10.

### Embodiment 20

The method of any one of embodiments 1-19, wherein v is 2-10.

### Embodiment 21

The method of any one of embodiments 1-20, wherein v is 2-5.

### Embodiment 22

The method of any one of embodiments 1-21, wherein v is 2.

### Embodiment 23

A method of treating cancer in a subject in need thereof, the method comprising administering to the subject a therapeutically effective amount of a peptidomimetic macrocycle and at least one additional pharmaceutically active agent, wherein the at least one additional pharmaceutically active agent:
(a) is selected from the group consisting of cobimetinib and binimetinib, or
(b) is a cyclin dependent kinase inhibitor (CDKI) and the CDKI and the peptidomimetic macrocycle are administered with a time separation of more than about 61 minutes;

wherein the peptidomimetic macrocycle comprises an amino acid sequence which is at least about 60% identical to an amino acid sequence chosen from the group consisting of the amino acid sequences in Table 1, Table 1a, Table 1b, or Table 1c and wherein the peptidomimetic macrocycle has the formula:

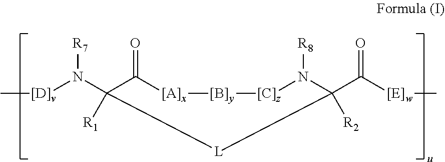

wherein:
each A, C, D, and E is independently an amino acid;
each B is independently an amino acid, amino acid analogue,

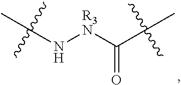

[—NH-L₃-CO—], [—NH-L₃-SO₂—], or [—NH-L₃-];
each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-; or at least one of R₁ and R₂ forms a macrocycle-forming linker L′ connected to the alpha position of one of said D or E amino acids;
each R₃ is independently hydrogen, alkyl, alkenyl, alkynyl, arylalkyl, heteroalkyl, cycloalkyl, heterocycloalkyl, cycloalkylalkyl, aryl, or heteroaryl, optionally substituted with R₅;
each L and L′ is independently a macrocycle-forming linker of the formula -L₁-L₂-;
each L₁, L₂, and L₃ is independently alkylene, alkenylene, alkynylene, heteroalkylene, cycloalkylene, heterocycloalkylene, arylene, heteroarylene, or [—R₄—K—R₄-]ₙ, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
each R₇ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
each R₈ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
each v and w is independently an integer from 1-1000;
u is an integer from 1-10;
each x, y, and z is independently an integer from 0-10;
n is an integer from 1-5; and
   wherein the peptidomimetic macrocycle is not a peptidomimetic macrocycle of Tables 2a or 2b.

### Embodiment 24

A method of treating cancer in a subject in need thereof, the method comprising administering to the subject a therapeutically effective amount of a peptidomimetic macrocycle and at least one additional pharmaceutically active agent, wherein the at least one additional pharmaceutically active agent:
(a) is selected from the group consisting of cobimetinib and binimetinib, or
(b) is a cyclin dependent kinase inhibitor (CDKI) and the CDKI and the peptidomimetic macrocycle are administered with a time separation of more than about 61 minutes;

wherein the peptidomimetic macrocycle comprises an amino acid sequence which is at least about 60% identical to an amino acid sequence chosen from the group consisting of the amino acid sequences in Table 1, Table 1a, Table 1b, or Table 1c, wherein the peptidomimetic macrocycle has the formula:

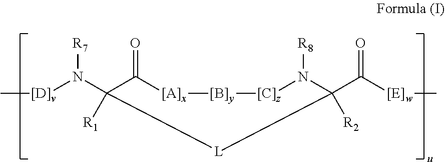

wherein:
each A, C, D, and E is independently an amino acid;
each B is independently an amino acid, amino acid analogue,

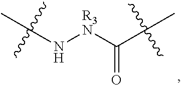

[—NH-L₃-CO—], [—NH-L₃-SO₂—], or [—NH-L₃-];
each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-; or at least one of R₁ and R₂ forms a macrocycle-forming linker L′ connected to the alpha position of one of said D or E amino acids;
each R₃ is independently hydrogen, alkyl, alkenyl, alkynyl, arylalkyl, heteroalkyl, cycloalkyl, heterocycloalkyl, cycloalkylalkyl, aryl, or heteroaryl, optionally substituted with R₅;
each L and L′ is independently a macrocycle-forming linker of the formula -L₁-L₂-;
each L₁, L₂, and L₃ is independently alkylene, alkenylene, alkynylene, heteroalkylene, cycloalkylene, heterocycloalkylene, arylene, heteroarylene, or [—R₄—K—R₄-]ₙ, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
each R₇ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
each R₈ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
each v and w is independently an integer from 1-1000;
u is an integer from 1-10;
each x, y, and z is independently an integer from 0-10;
n is an integer from 1-5;
wherein w>2 and each of the first two amino acid represented by E comprises an uncharged side chain or a negatively charged side chain,
with the proviso that the peptidomimetic macrocycle is not a peptidomimetic macrocycle of Table 2a and does not have the sequence:

(SEQ ID NO: 762)
Ac-RTQATF$r8NQWAibANle$TNAibTR-NH₂,
(SEQ ID NO: 813)
Ac-Sr8SQQTFS$LWRLLAibQN-NH₂,
(SEQ ID NO: 814)
Ac-QSQ$r8TFSNLW$LLAibQN-NH₂,
(SEQ ID NO: 816)
Ac-QS$r5QTFStNLW$LLAibQN-NH₂,
or
(SEQ ID NO: 896)
Ac-QSQQ$r8FSNLWR$LAibQN-NH₂,

wherein Aib represents 2-aminoisobutyric acid, $ represents an alpha-Me S5-pentenyl-alanine olefin amino acid connected to another amino acid side chain by an all-carbon crosslinker comprising one double bond, $r5 represents an alpha-Me R₅-pentenyl-alanine olefin amino acid connected to another amino acid side chain by an all-carbon comprising one double bond, and $r8 represents an alpha-Me R₈-octenyl-alanine olefin amino acid connected to another amino acid side chain by an all-carbon crosslinker comprising one double bond.

### Embodiment 25

The method of embodiments 24 or 25, wherein each E is independently an amino acid selected from Ala (alanine), D-Ala (D-alanine), Aib (α-aminoisobutyric acid), Sar (N-methyl glycine), and Ser (serine).

### Embodiment 26

The method of any one of embodiments 24-25, wherein the first C-terminal amino acid and/or the second C-terminal amino acid represented by E comprise a hydrophobic side chain.

### Embodiment 27

The method of embodiment 27, wherein the hydrophobic chain is a large hydrophobic side chain.

### Embodiment 28

A method of treating cancer in a subject in need thereof, the method comprising administering to the subject a therapeutically effective amount of a peptidomimetic macrocycle and at least one additional pharmaceutically active agent, wherein the at least one additional pharmaceutically active agent:
(a) is selected from the group consisting of cobimetinib and binimetinib, or
(b) is a cyclin dependent kinase inhibitor (CDKI) and the CDKI and the peptidomimetic macrocycle are administered with a time separation of more than about 61 minutes;

wherein the peptidomimetic macrocycle comprises an amino acid sequence which is at least about 60% identical to an amino acid sequence chosen from the group consisting of the amino acid sequences in Table 1, Table 1a, Table 1b, or Table 1c, wherein the peptidomimetic macrocycle has the formula:

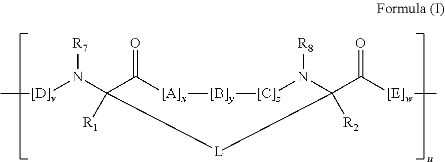

wherein:
each A, C, D, and E is independently an amino acid;
each B is independently an amino acid, amino acid analogue,

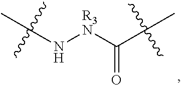

[—NH-L₃-CO—], [—NH-L₃-SO₂—], or [—NH-L₃-];
each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-; or at least one of R₁ and R₂ forms a macrocycle-forming linker L′ connected to the alpha position of one of said D or E amino acids;
each R₃ is independently hydrogen, alkyl, alkenyl, alkynyl, arylalkyl, heteroalkyl, cycloalkyl, heterocycloalkyl, cycloalkylalkyl, aryl, or heteroaryl, optionally substituted with R₅;
each L and L′ is independently a macrocycle-forming linker of the formula -L₁-L₂-;
each L₁, L₂, and L₃ is independently alkylene, alkenylene, alkynylene, heteroalkylene, cycloalkylene, heterocycloalkylene, arylene, heteroarylene, or [—R₄—K—R₄-]ₙ, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
each R₇ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
each R₅ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
each v and w is independently an integer from 1-1000;
u is an integer from 1-10;
each x, y, and z is independently an integer from 0-10;
n is an integer from 1-5; and
w>2,

wherein the third amino acid represented by E comprises a large hydrophobic side chain, with the proviso that the peptidomimetic macrocycle is not a peptidomimetic macrocycle of Table 2a and does not have the sequence of: Ac-Q$r8QQTFSN$WRLLAibQN-NH₂ (SEQ ID NO: 895).

### Embodiment 29

The method of embodiment 28, wherein each E other than the third amino acid represented by E is an amino acid selected from Ala (alanine), D-Ala (D-alanine), Aib (α-aminoisobutyric acid), Sar (N-methyl glycine), and Ser (serine).

### Embodiment 30

The method of any one of embodiments 23-29, wherein w is 3-10.

### Embodiment 31

The method of any one of embodiments 23-30, wherein w is 3-6.

### Embodiment 32

The method of any one of embodiments 23-29, wherein w is 6-10.

### Embodiment 33

The method of any one of embodiments 23-32, wherein w is 6.

### Embodiment 34

The method of any one of embodiments 24-33, wherein v is 1-10.

### Embodiment 35

The method of any one of embodiments 23-34, wherein v is 3-10.

### Embodiment 36

The method of any one of embodiments 23-35, wherein v is 3-5.

### Embodiment 37

The method of any one of embodiments 23-36, wherein v is 3.

### Embodiment 38

The method of any one of embodiments 34-37, wherein [D]ᵥ is -Leu₁-Thr₂-Phe₃.

### Embodiment 39

The method of any one of embodiments 28-38, wherein each of the first two amino acid represented by E comprises an uncharged side chain or a negatively charged side chain.

### Embodiment 40

The method of any one of embodiments 28-38, wherein the third amino acid represented by E is an amino acid selected from the group consisting of: isoleucine (I), leucine (L), methionine (M), phenylalanine (F), tryptophan (W), and tyrosine (Y).

### Embodiment 41

The method of any one of embodiments 1-40, wherein L₁ and L₂ are independently alkylene, alkenylene, alkynylene, heteroalkylene, cycloalkylene, heterocycloalkylene, arylene, or heteroarylene, each being optionally substituted with R₅.

### Embodiment 42

The method of any one of embodiments 1-40, wherein L₁ and L₂ are independently alkylene or alkenylene.

### Embodiment 43

The method of any one of embodiments 1-40, wherein L is alkylene, alkenylene, or alkynylene.

### Embodiment 44

The method of any one of embodiments 1-43, wherein L is alkylene.

### Embodiment 45

The method of any one of embodiments 1-44, wherein L is C₃-C₁₆ alkylene.

### Embodiment 46

The method of any one of embodiments 1-44, wherein L is C₁₀-C₁₄ alkylene.

### Embodiment 47

The method of any one of embodiments 1-46, wherein R₁ and R₂ are independently —H, alkyl, alkenyl, alkynyl, arylalkyl, cycloalkyl, cycloalkylalkyl, heteroalkyl, or heterocycloalkyl, unsubstituted or substituted with halo.

### Embodiment 48

The method of any one of embodiments 1-47, wherein R₁ and R₂ are H.

### Embodiment 49

The method of any one of embodiments 1-48, wherein R₁ and R₂ are independently alkyl.

### Embodiment 50

The method of any one of embodiments 1-49, wherein R₁ and R₂ are methyl.

### Embodiment 51

The method of any one of embodiments 1-50, wherein x+y+z=6.

### Embodiment 52

The method of any one of embodiments 1-51, wherein u is 1.

### Embodiment 53

The method of any one of embodiments 1-52, wherein the peptidomimetic macrocycle is not a macrocycle of Table 2a or Table 2b.

### Embodiment 54

The method of any one of embodiments 1-53, wherein each E is Ser or Ala or an analogue thereof.

### Embodiment 55

The method of any one of embodiments 1-54, wherein the peptidomimetic macrocycle comprises at least one amino acid which is an amino acid analogue.

### Embodiment 56

A method of treating cancer in a subject in need thereof, the method comprising administering to the subject

(a) a therapeutically effective amount of a p53 agent that
(i) inhibits the interaction between p53 and MDM2 and/or p53 and MDMX, and/or
(ii) modulates the activity of p53 and/or MDM2 and/or MDMX; and

(b) at least one additional pharmaceutically active agent, wherein the at least one additional pharmaceutically active agent
(i) modulates the activity of CDK4 and/or CDK6, and/or
(ii) inhibits CDK4 and/or CDK6;
   wherein the at least one additional pharmaceutically active agent and the peptidomimetic macrocycle are administered with a time separation of more than about 61 minutes.

### Embodiment 57

The method of embodiment 56, wherein the p53 agent antagonizes an interaction between p53 and MDM2 proteins and/or between p53 and MDMX proteins.

### Embodiment 58

The method of embodiments 56 or 57, wherein the at least one additional pharmaceutically active agent binds to CDK4 and/or CDK6.

### Embodiment 59

The method of any one of embodiments 56-58, wherein the p53 agent is selected from the group consisting of a small organic or inorganic molecule; a saccharine; an oligosaccharide; a polysaccharide; a peptide, a protein, a peptide analogue, a peptide derivative; an antibody, an antibody fragment, a peptidomimetic; a peptidomimetic macrocycle of any one of embodiments 1-55 a nucleic acid; a nucleic acid analogue, a nucleic acid derivative; an extract made from biological materials; a naturally occurring or synthetic composition; and any combination thereof.

### Embodiment 60

The method of any one of embodiments 56-59, wherein the p53 agent is selected from the group consisting of RG7388 (RO5503781, idasanutlin); RG7112 (RO5045337); nutlin3a; nutlin3b; nutlin3; nutlin2; spirooxindole containing small molecules; 1,4-diazepines; 1,4-benzodiazepine-2,5-dione compounds; WK23; WK298; SJ172550; RO2443; RO5963; RO5353; RO2468; MK8242 (SCH900242); MI888; MI773 (SAR405838); NVPCGM097; DS3032b; AM8553; AMG232; NSC207895 (XI006); JNJ26854165 (serdemetan); RITA (NSC652287); YH239EE; and any combination thereof.

### Embodiment 61

The method of any one of embodiments 56-60, wherein the at least one additional pharmaceutically active agent is selected from the group consisting of a small organic or inorganic molecule; a saccharine; an oligosaccharide; a polysaccharide; a peptide, a protein, a peptide analogue, a peptide derivative; an antibody, an antibody fragment, a peptidomimetic; a peptidomimetic macrocycle of any one of embodiments 1-55; a nucleic acid; a nucleic acid analogue, a nucleic acid derivative; an extract made from biological materials; a naturally occurring or synthetic composition; and any combination thereof.

### Embodiment 62

The method of any one of embodiments 1-61, wherein the at least one additional pharmaceutically active agent is selected from the group consisting of palbociclib (PD0332991); abemaciclib (LY2835219); ribociclib (LEE 011); voruciclib (P1446A-05); fascaplysin; arcyriaflavin; 2-bromo-12,13-dihydro-5H-indolo[2,3-a]pyrrolo[3,4-c]carbazole-5,7(6H)-dione; 3-amino thioacridone (3-ATA), trans-4-((6-(ethylamino)-2-((1-(phenylmethyl)-1H-indol-5-yl)amino)-4-pyrimidinyl)amino)-cyclohexano (CINK4); 1,4-dimethoxyacridine-9(10H)-thione (NSC 625987); 2-methyl-5-(p-tolylamino)benzo[d]thiazole-4,7-dione (ryuvidine); and flavopiridol (alvocidib); seliciclib; dinaciclib; milciclib; roniciclib; atuveciclib; briciclib; riviciclib; trilaciclib (G1T28); and any combination thereof.

### Embodiment 63

The method of embodiments 1 or 2, wherein the peptidomimetic macrocycle is (SEQ ID NO: 163)

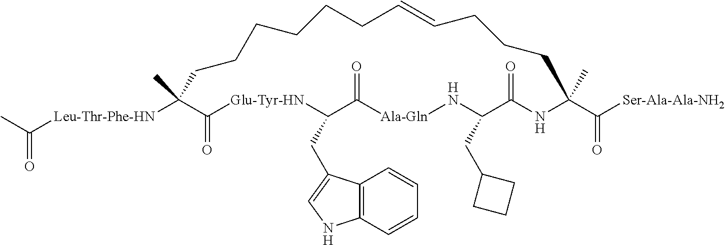

or a pharmaceutically acceptable salt thereof.

### Embodiment 64

The method of embodiments 1 or 2, wherein the peptidomimetic macrocycle is (SEQ ID NO: 124)

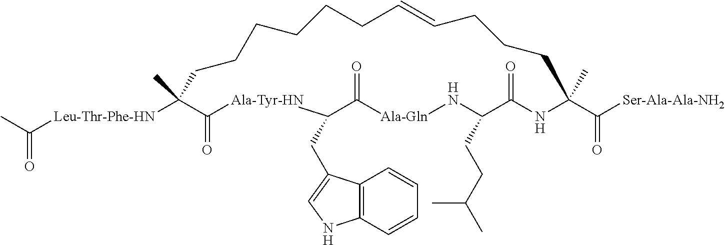

or a pharmaceutically acceptable salt thereof.

### Embodiment 65

The method of embodiments 1 or 2, wherein the peptidomimetic macrocycle is (SEQ ID NO: 123):

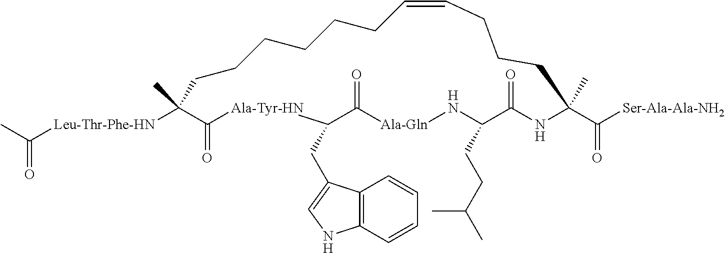

or a pharmaceutically acceptable salt thereof.

### Embodiment 66

The method of embodiments 1 or 2, wherein the peptidomimetic macrocycle is (SEQ ID NO: 108)

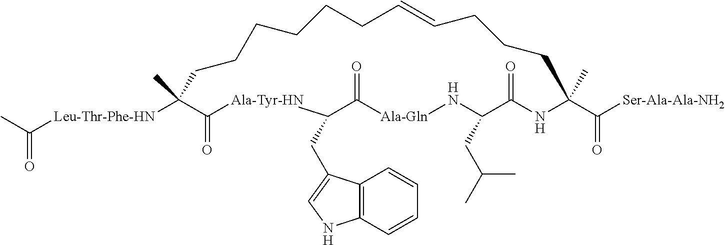

or a pharmaceutically acceptable salt thereof.

### Embodiment 67

The method of embodiments 1 or 2, wherein the peptidomimetic macrocycle is (SEQ ID NO: 397)

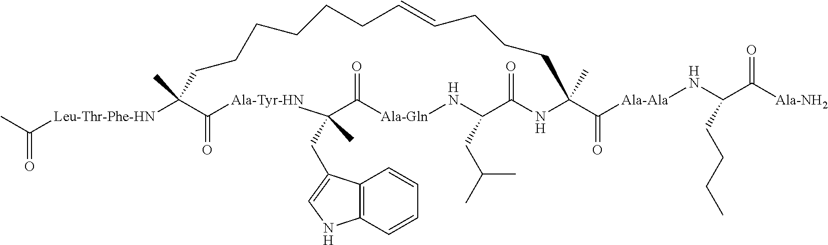

or a pharmaceutically acceptable salt thereof.

### Embodiment 68

The method of embodiments 1 or 2, wherein the peptidomimetic macrocycle is (SEQ ID NO: 340)

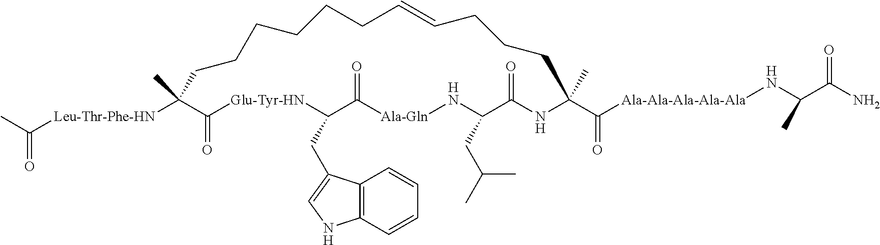

or a pharmaceutically acceptable salt thereof.

### Embodiment 69

The method of embodiments 1 or 2, wherein the peptidomimetic macrocycle is (SEQ ID NO: 454)

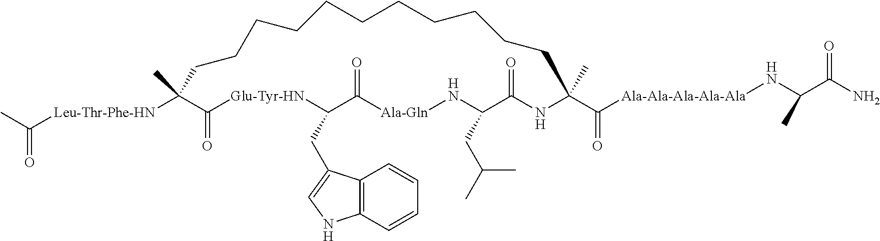

or a pharmaceutically acceptable salt thereof.

### Embodiment 70

The method of embodiments 1 or 2, wherein the peptidomimetic macrocycle is (SEQ ID NO: 360)

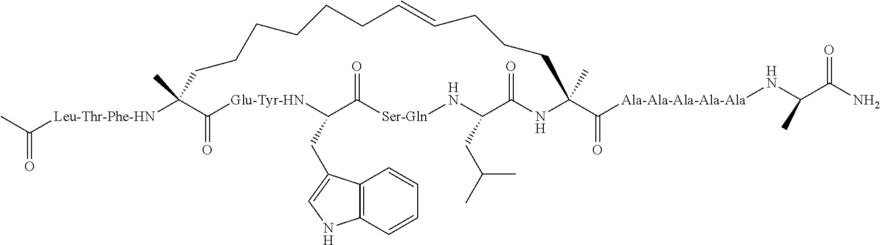

or a pharmaceutically acceptable salt thereof.

### Embodiment 71

The method of embodiments 1 or 2, wherein the peptidomimetic macrocycle is (SEQ ID NO: 80)

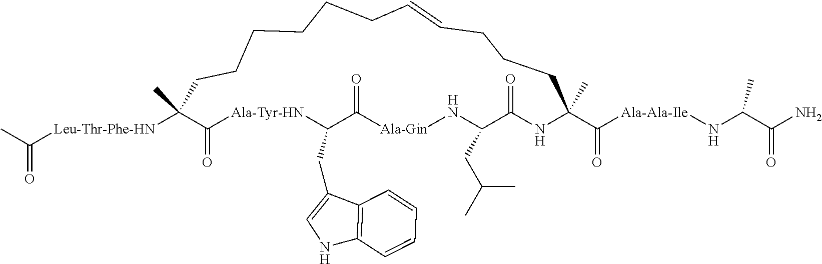

or a pharmaceutically acceptable salt thereof.

### Embodiment 72

The method of embodiments 1 or 2, wherein the peptidomimetic macrocycle is (SEQ ID NO: 78)

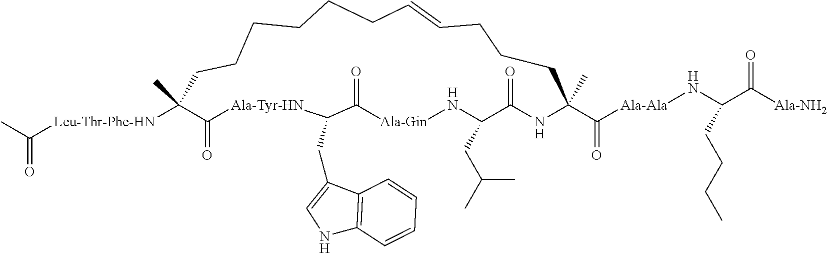

or a pharmaceutically acceptable salt thereof.

### Embodiment 73

The method of embodiments 1 or 2, wherein the peptidomimetic macrocycle is (SEQ ID NO: 16)

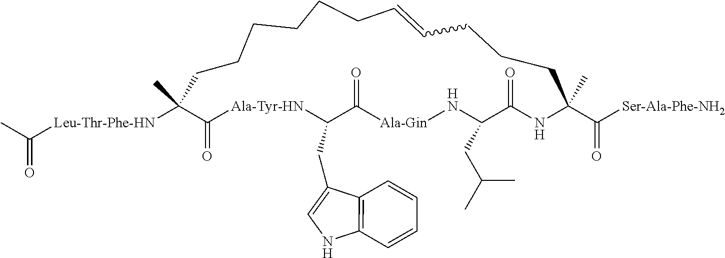

or a pharmaceutically acceptable salt thereof.

### Embodiment 74

The method of embodiments 1 or 2, wherein the peptidomimetic macrocycle is (SEQ ID NO: 169)

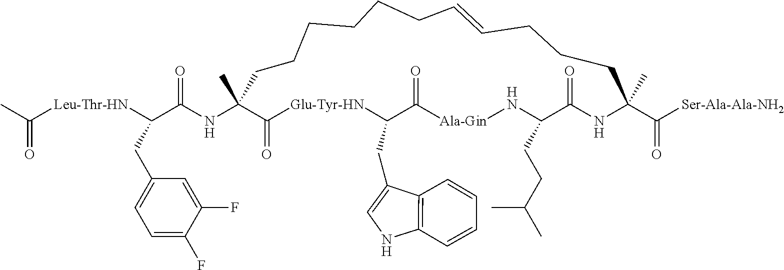

or a pharmaceutically acceptable salt thereof.

### Embodiment 75

The method of embodiments 1 or 2, wherein the peptidomimetic macrocycle is (SEQ ID NO: 324)

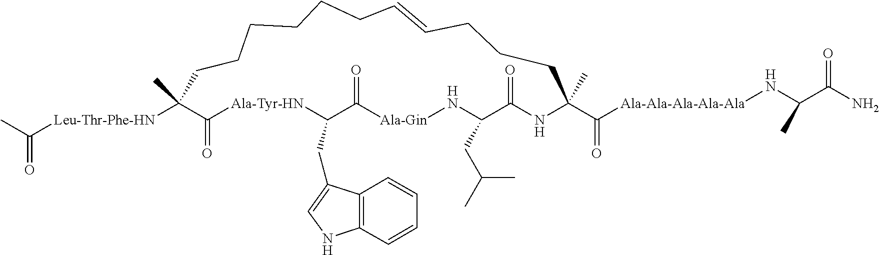

or a pharmaceutically acceptable salt thereof.

### Embodiment 76

The method of embodiments 1 or 2, wherein the peptidomimetic macrocycle is (SEQ ID NO: 258)

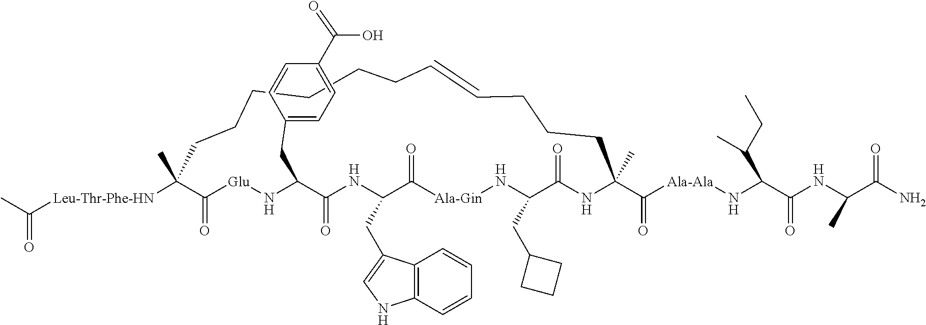

or a pharmaceutically acceptable salt thereof.

### Embodiment 77

The method of embodiments 1 or 2, wherein the peptidomimetic macrocycle is (SEQ ID NO: 446)

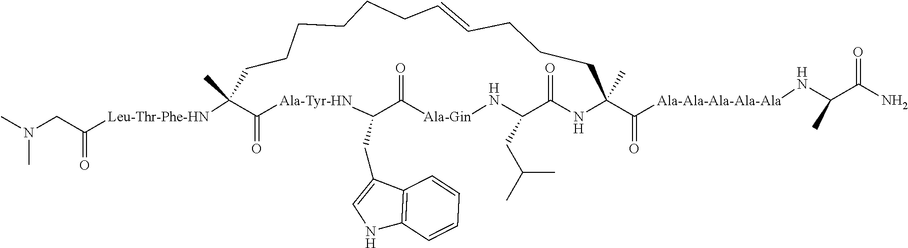

or a pharmaceutically acceptable salt thereof.

### Embodiment 78

The method of embodiments 1 or 2, wherein the peptidomimetic macrocycle is (SEQ ID NO: 358)

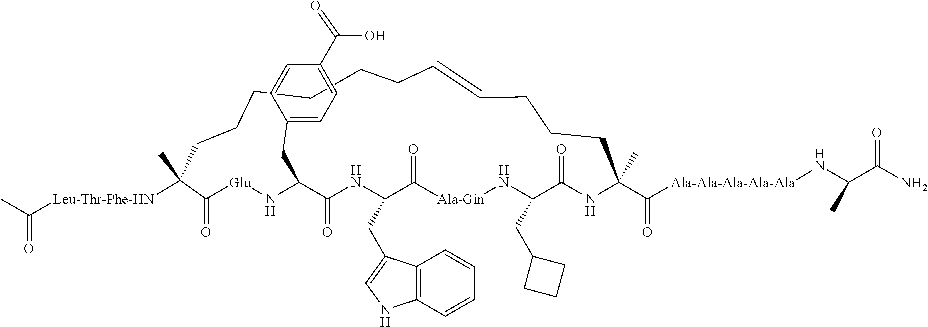

or a pharmaceutically acceptable salt thereof.

### Embodiment 79

The method of embodiments 1 or 2, wherein the peptidomimetic macrocycle is (SEQ ID NO: 464)

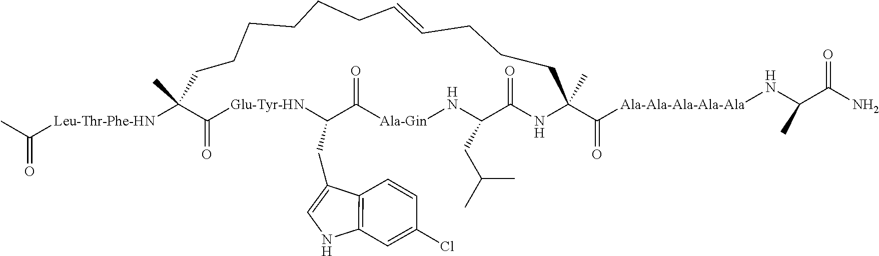

or a pharmaceutically acceptable salt thereof.

### Embodiment 80

The method of embodiments 1 or 2, wherein the peptidomimetic macrocycle is (SEQ ID NO: 466)

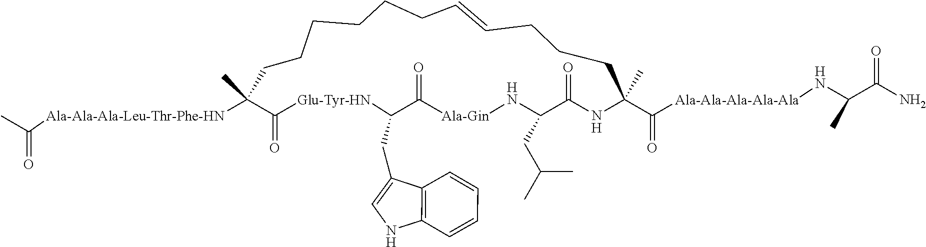

or a pharmaceutically acceptable salt thereof.

### Embodiment 81

The method of embodiments 1 or 2, wherein the peptidomimetic macrocycle is (SEQ ID NO: 467)

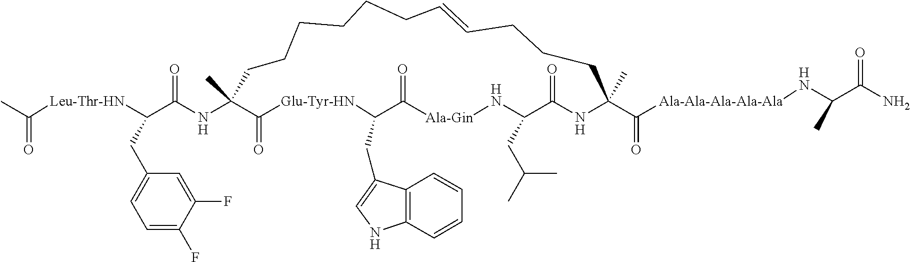

or a pharmaceutically acceptable salt thereof.

### Embodiment 82

The method of embodiments 1 or 2, wherein the peptidomimetic macrocycle is (SEQ ID NO: 376)

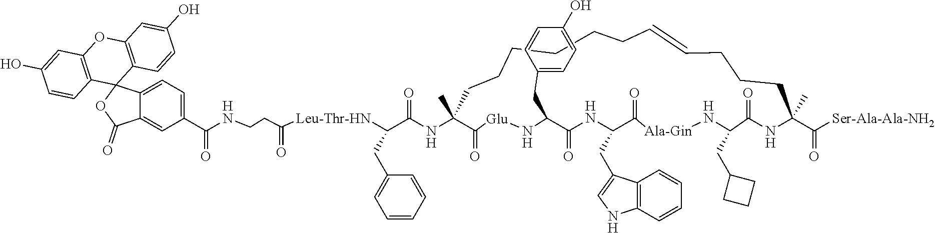

or a pharmaceutically acceptable salt thereof.

### Embodiment 83

The method of embodiments 1 or 2, wherein the peptidomimetic macrocycle is (SEQ ID NO: 471)

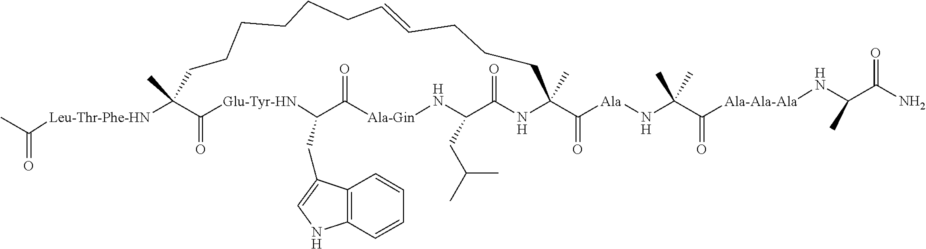

or a pharmaceutically acceptable salt thereof.

### Embodiment 84

The method of embodiments 1 or 2, wherein the peptidomimetic macrocycle is (SEQ ID NO: 473)

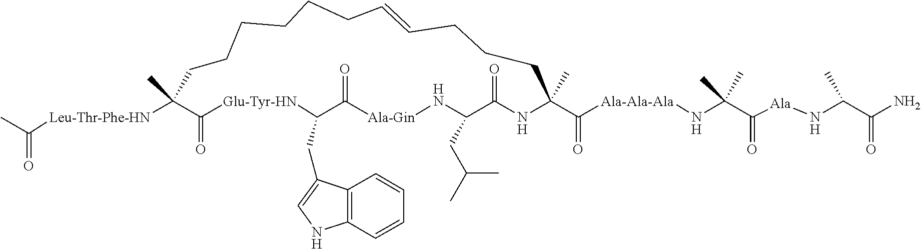

or a pharmaceutically acceptable salt thereof.

### Embodiment 85

The method of embodiments 1 or 2, wherein the peptidomimetic macrocycle is (SEQ ID NO: 475)

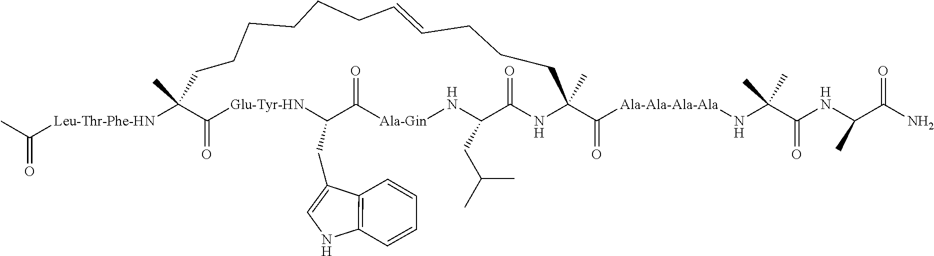

or a pharmaceutically acceptable salt thereof.

### Embodiment 86

The method of embodiments 1 or 2, wherein the peptidomimetic macrocycle is (SEQ ID NO: 476)

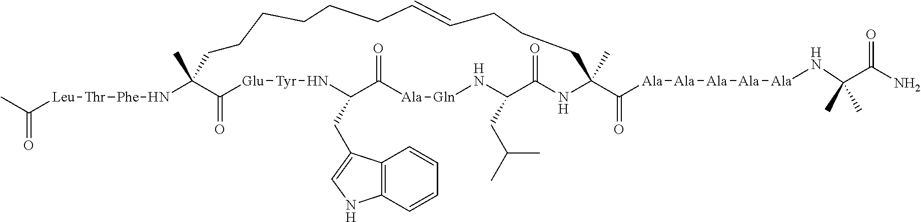

or a pharmaceutically acceptable salt thereof.

### Embodiment 87

The method of embodiments 1 or 2, wherein the peptidomimetic macrocycle is (SEQ ID NO: 481)

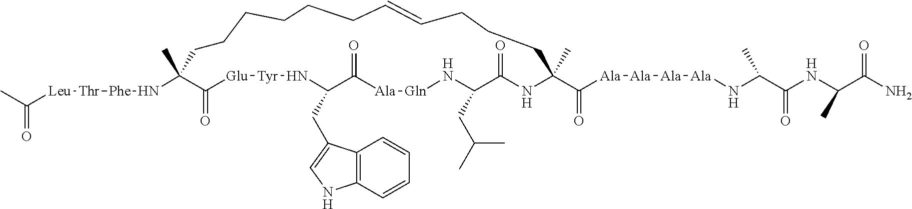

or a pharmaceutically acceptable salt thereof.

### Embodiment 88

The method of embodiments 1 or 2, wherein the peptidomimetic macrocycle is (SEQ ID NO: 482)

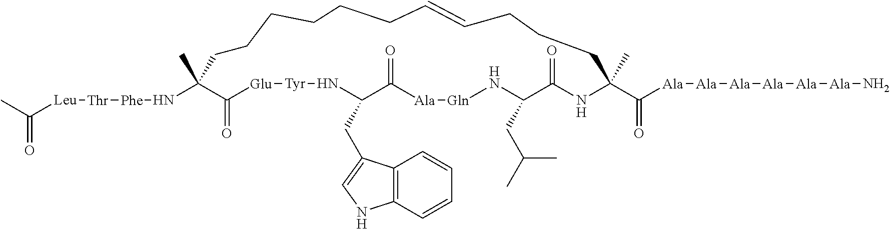

or a pharmaceutically acceptable salt thereof.

### Embodiment 89

The method of embodiments 1 or 2, wherein the peptidomimetic macrocycle is (SEQ ID NO: 487)

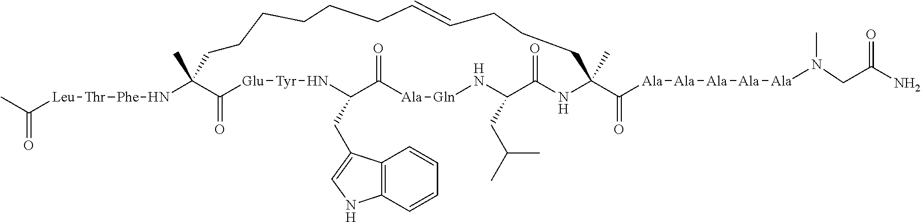

or a pharmaceutically acceptable salt thereof.

### Embodiment 90

The method of embodiments 1 or 2, wherein the peptidomimetic macrocycle is (SEQ ID NO: 572)

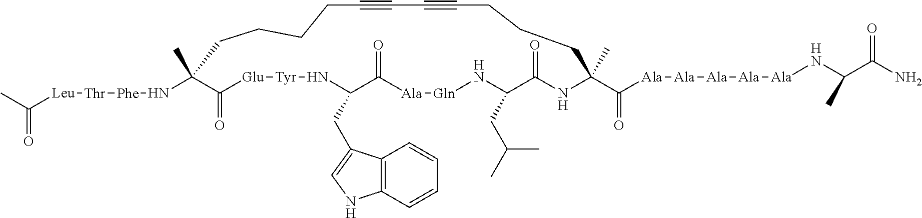

or a pharmaceutically acceptable salt thereof.

### Embodiment 91

The method of embodiments 1 or 2, wherein the peptidomimetic macrocycle is (SEQ ID NO: 572)

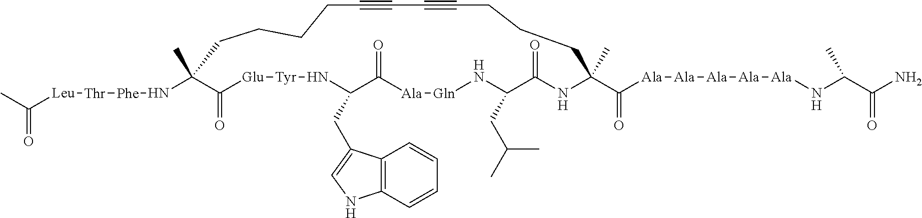

or a pharmaceutically acceptable salt thereof.

### Embodiment 92

The method of embodiments 1 or 2, wherein the peptidomimetic macrocycle is (SEQ ID NO: 1500)

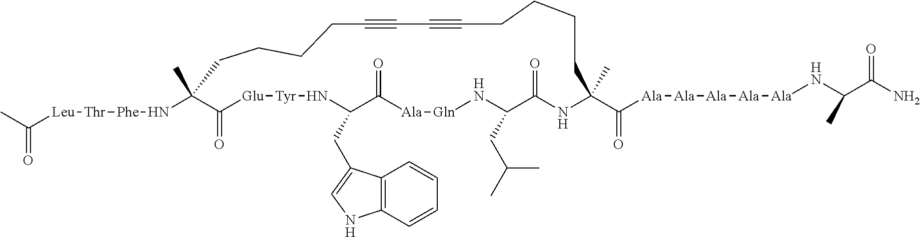

or a pharmaceutically acceptable salt thereof.

### Embodiment 94

A method of modulating the activity of p53 and/or MDM2 and/or MDMX in a subject in need thereof comprising administering to the subject a therapeutically-effective amount of a peptidomimetic macrocycle and at least one additional pharmaceutically active agent, wherein the at least one additional pharmaceutically active agent:
(a) is selected from the group consisting of cobimetinib and binimetinib, or
(b) is a cyclin dependent kinase inhibitor (CDKI) and the CDKI and the peptidomimetic macrocycle are administered with a time separation of more than about 61 minutes;

wherein the peptidomimetic macrocycle comprises an amino acid sequence which is at least about 60% identical to an amino acid sequence in any of Table 1, Table 1a, Table 1b, and Table 1c, wherein the peptidomimetic macrocycle has the formula:

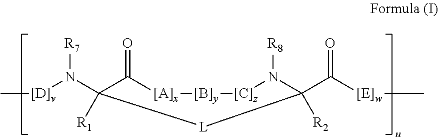

or pharmaceutically acceptable salt thereof, wherein:
each A, C, D, and E is independently an amino acid;
each B is independently an amino acid,

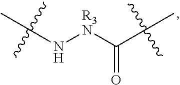

[—NH-L₃-CO—], [—NH-L₃-SO₂—], or [—NH-L₃-];
each R₁ and R₂ is independently hydrogen, alkyl, alkenyl, alkynyl, arylalkyl, cycloalkyl, cycloalkylalkyl, heteroalkyl, or heterocycloalkyl, unsubstituted or substituted with halo-; or forms a macrocycle-forming linker L′ connected to the alpha position of one of said D or E amino acids;
each R₃ is independently hydrogen, alkyl, alkenyl, alkynyl, arylalkyl, heteroalkyl, cycloalkyl, heterocycloalkyl, cycloalkylalkyl, aryl, or heteroaryl, optionally substituted with R₅;
each L and L′ is independently a macrocycle-forming linker of the formula -L₁-L₂-;
each L₁, L₂, and L₃ is independently alkylene, alkenylene, alkynylene, heteroalkylene, cycloalkylene, heterocycloalkylene, arylene, heteroarylene, or [—R₄—K—R₄-]ₙ, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently hydrogen, alkyl, alkenyl, alkynyl, arylalkyl, cycloalkylalkyl, heterocycloalkyl, a fluorescent moiety, a radioisotope or a therapeutic agent;
each R₇ is independently hydrogen, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
each R₈ is independently hydrogen, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
each v is independently an integer from 1-1000;
each w is independently an integer from 1-1000;
u is an integer from 1-10;
each x, y and z is independently an integer from 0-10; and
each n is independently an integer from 1-5.

### Embodiment 95

A method of antagonizing an interaction between p53 and MDM2 proteins and/or between p53 and MDMX proteins in a subject in need thereof comprising administering to the subject a therapeutically-effective amount of a peptidomimetic macrocycle and at least one additional pharmaceutically active agent, wherein the at least one additional pharmaceutically active agent:
(a) is selected from the group consisting of cobimetinib and binimetinib, or
(b) is a cyclin dependent kinase inhibitor (CDKI) and the CDKI and the peptidomimetic macrocycle are administered with a time separation of more than about 61 minutes;

wherein the peptidomimetic macrocycle comprises an amino acid sequence which is at least about 60% identical to an amino acid sequence in any of Table 1, Table 1a, Table 1b, and Table 1c and wherein the peptidomimetic macrocycle has the formula:

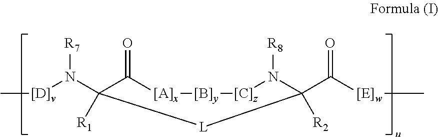

or pharmaceutically acceptable salt thereof, wherein:
each A, C, D, and E is independently an amino acid;
each B is independently an amino acid,

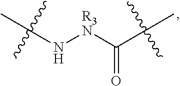

[—NH-L₃-CO—], [—NH-L₃-SO₂—], or [—NH-L₃-];
each R₁ and R₂ is independently hydrogen, alkyl, alkenyl, alkynyl, arylalkyl, cycloalkyl, cycloalkylalkyl, heteroalkyl, or heterocycloalkyl, unsubstituted or substituted with halo-; or forms a macrocycle-forming linker L′ connected to the alpha position of one of said D or E amino acids;
each R₃ is independently hydrogen, alkyl, alkenyl, alkynyl, arylalkyl, heteroalkyl, cycloalkyl, heterocycloalkyl, cycloalkylalkyl, aryl, or heteroaryl, optionally substituted with R₅;
each L and L′ is independently a macrocycle-forming linker of the formula -L₁-L₂-;
each L₁, L₂, and L₃ is independently alkylene, alkenylene, alkynylene, heteroalkylene, cycloalkylene, heterocycloalkylene, arylene, heteroarylene, or [—R₄—K—R₄-]ₙ, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently hydrogen, alkyl, alkenyl, alkynyl, arylalkyl, cycloalkylalkyl, heterocycloalkyl, a fluorescent moiety, a radioisotope or a therapeutic agent;
each R₇ is independently hydrogen, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
each R₈ is independently hydrogen, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
each v is independently an integer from 1-1000;
each w is independently an integer from 1-1000;
u is an integer from 1-10;
each x, y and z is independently an integer from 0-10; and
each n is independently an integer from 1-5.

### Embodiment 96

The method of any one of embodiments 1-95, wherein the cancer is selected from the group consisting of head and neck cancer, melanoma, lung cancer, breast cancer, colon cancer, ovarian cancer, NSCLC, stomach cancer, prostate cancer, leukemia, lymphoma, mesothelioma, renal cancer, non-Hodgkin lymphoma (NHL), and glioma.

### Embodiment 97

The method of any one of embodiments 1-96, wherein, a sub-therapeutic amount of the at least one additional pharmaceutically active agent is administered.

### Embodiment 98

The method of any one of embodiments 1-97, wherein a therapeutic amount of the at least one additional pharmaceutically active agent is administered.

### Embodiment 99

The method of any one of embodiments 1-98, wherein the at least one additional pharmaceutically active agent comprises cobimetinib or binimetinib.

### Embodiment 100

The method of any one of embodiments 1-98, wherein the at least one additional pharmaceutically active agent comprises the cyclin dependent kinase inhibitor (CDKI) and the CDKI and the peptidomimetic macrocycle are administered with a time separation of more than about 61 minutes.

### Embodiment 101

The method of any one of embodiments 1-98 or 100, wherein the at least one additional pharmaceutically active agent comprises palbociclib (PD0332991); abemaciclib (LY2835219); ribociclib (LEE 011); voruciclib (P1446A-05); fascaplysin; arcyriaflavin; 2-bromo-12,13-dihydro-5H-indolo[2,3-a]pyrrolo[3,4-c]carbazole-5,7(6H)-dione; 3-amino thioacridone (3-ATA), trans-4-((6-(ethylamino)-2-((1-(phenylmethyl)-1H-indol-5-yl)amino)-4-pyrimidinyl)amino)-cyclohexano (CINK4); 1,4-dimethoxyacridine-9(10H)-thione (NSC 625987); 2-methyl-5-(p-tolylamino)benzo[d]thiazole-4,7-dione (ryuvidine); and flavopiridol (alvocidib); seliciclib; dinaciclib; milciclib; roniciclib; atuveciclib; briciclib; riviciclib; trilaciclib; and any combination thereof.

### Embodiment 102

The method of embodiment 100 or 101, wherein the peptidomimetic macrocycle is administered at least 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before the cyclin dependent kinase inhibitor is administered.

### Embodiment 103

The method of embodiment 100 or 101, wherein the peptidomimetic macrocycle is administered at most 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before the cyclin dependent kinase inhibitor is administered.

### Embodiment 104

The method of embodiment 100 or 101, wherein the peptidomimetic macrocycle is administered about 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before the cyclin dependent kinase inhibitor is administered.

### Embodiment 105

The method of embodiment 100 or 101, wherein the peptidomimetic macrocycle is administered at least 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, after the cyclin dependent kinase inhibitor is administered.

### Embodiment 106

The method of embodiment 100 or 101, wherein the peptidomimetic macrocycle is administered at most 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, after the cyclin dependent kinase inhibitor is administered.

### Embodiment 107

The method of embodiment 100 or 101, wherein the peptidomimetic macrocycle is administered about 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, after the cyclin dependent kinase inhibitor is administered.

### Embodiment 108

The method of any one of embodiments 1-107, wherein an additional therapeutic agent is administered.

### Embodiment 109

The method of any one of embodiments 1-108, wherein the subject comprises cancer cells that overexpress PD-L1.

### Embodiment 110

The method of any one of embodiments 1-109, wherein the subject comprises cancer cells that overexpress PD-1.

### Embodiment 111

The method of any one of embodiments 1-110, wherein the subject comprises cancer cells that overexpress miR-34.

### Embodiment 112

The method of any one of embodiments 108-111, wherein the additional therapeutic agent is a PD-1 antagonist.

### Embodiment 113

The method of any one of embodiments 108-112, wherein the additional therapeutic agent is a PD-L1 antagonist.

### Embodiment 114

The method of any one of embodiments 108-113, wherein the additional therapeutic agent is an agent that blocks the binding of PD-L1 to PD-1.

### Embodiment 115

The method of any one of embodiments 108-114, wherein the additional therapeutic agent specifically binds to PD-1.

### Embodiment 116

The method of any one of embodiments 108-115, wherein the additional therapeutic agent specifically binds to PD-L1.

### Embodiment 117

The method of any one of embodiments 1-116, wherein PD-L1 expression is downregulated.

### Embodiment 118

The method of any one of embodiments 1-117, wherein PD-1 expression is downregulated.

### Embodiment 119

The method of any one of embodiments 1-118, wherein S-phase is inhibited.

### Embodiment 120

The method of any one of embodiments 1-119, wherein M-phase is inhibited.

### Embodiment 121

The method of any one of embodiments 1-120, wherein the peptidomimetic macrocycle antagonizes an interaction between p53 and MDM2 proteins.

### Embodiment 122

The method of any one of embodiments 1-121, wherein the peptidomimetic macrocycle antagonizes an interaction between p53 and MDMX proteins.

### Embodiment 123

The method of any one of embodiments 1-122, wherein the peptidomimetic macrocycle antagonizes an interaction between p53 and MDM2 proteins and p53 and MDMX proteins.

### Embodiment 124

The method of any one of embodiments 1-123, wherein the peptidomimetic macrocycle antagonizes an interaction between p53 and MDM2 proteins and p53 and MDMX proteins.

### Embodiment 201

A method of treating a condition in a subject in need thereof, the method comprising administering to the subject a therapeutically-effective amount of a peptidomimetic macrocycle and at least one pharmaceutically-active agent, wherein the peptidomimetic macrocycle and the at least one pharmaceutically-active agent are administered with a time separation of more than 61 minutes.

### Embodiment 202

The method of embodiment 201, wherein the peptidomimetic macrocycle is of the formula:

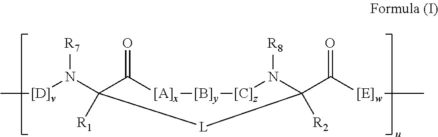

or pharmaceutically acceptable salt thereof, wherein:
each A, C, D, and E is independently an amino acid;
each B is independently an amino acid,

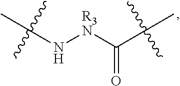

[—NH-L₃-CO—], [—NH-L₃-SO₂—], or [—NH-L₃-];
each R₁ and R₂ is independently hydrogen, alkyl, alkenyl, alkynyl, arylalkyl, cycloalkyl, cycloalkylalkyl, heteroalkyl, or heterocycloalkyl, unsubstituted or substituted with halo-; or forms a macrocycle-forming linker L′ connected to the alpha position of one of said D or E amino acids;
each R₃ is independently hydrogen, alkyl, alkenyl, alkynyl, arylalkyl, heteroalkyl, cycloalkyl, heterocycloalkyl, cycloalkylalkyl, aryl, or heteroaryl, optionally substituted with R₅;
each L and L′ is independently a macrocycle-forming linker of the formula -L₁-L₂-;
each L₁, L₂, and L₃ is independently alkylene, alkenylene, alkynylene, heteroalkylene, cycloalkylene, heterocycloalkylene, arylene, heteroarylene, or [—R₄—K—R₄-]ₙ, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently hydrogen, alkyl, alkenyl, alkynyl, arylalkyl, cycloalkylalkyl, heterocycloalkyl, a fluorescent moiety, a radioisotope or a therapeutic agent;
each R₇ is independently hydrogen, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
each R₈ is independently hydrogen, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
each v is independently an integer from 1-1000;
each w is independently an integer from 1-1000;
u is 1, 2, 3, 4, 5, 6, 7, 8, 9, or 10;
each x, y and z is independently 1, 2, 3, 4, 5, 6, 7, 8, 9, or 10; and
each n is independently 1, 2, 3, 4, or 5.

### Embodiment 203

The method of embodiment 202, wherein v is 3-10.

### Embodiment 204

The method of embodiments 202 or 203, wherein v is 3.

### Embodiment 205

The method of any one of embodiments 202-204, wherein w is 3-10.

### Embodiment 206

The method of any one of embodiments 202-205, wherein w is 6.

### Embodiment 207

The method of any one of embodiments 202-206, wherein x+y+z=6.

### Embodiment 208

The method of any one of embodiments 202-207, wherein each L₁ and L₂ is independently alkylene, alkenylene, alkynylene, heteroalkylene, cycloalkylene, heterocycloalkylene, arylene, or heteroarylene.

### Embodiment 209

The method of any one of embodiments 202-208, wherein each L₁ and L₂ is independently alkylene or alkenylene.

### Embodiment 210

The method of any one of embodiments 202-209, wherein each R₁ and R₂ is independently hydrogen, alkyl, alkenyl, alkynyl, arylalkyl, cycloalkyl, cycloalkylalkyl, heteroalkyl, or heterocycloalkyl, unsubstituted or substituted with halo-.

### Embodiment 211

The method of any one of embodiments 202-210, wherein each R₁ and R₂ is independently hydrogen.

### Embodiment 212

The method of any one of embodiments 202-210, wherein each R₁ and R₂ is independently alkyl.

### Embodiment 213

The method of any one of embodiments 202-210 or 212, wherein each R₁ and R₂ is independently methyl.

### Embodiment 214

The method of any one of embodiments 202-214, wherein u is 1.

### Embodiment 215

The method of any one of embodiments 202-214, wherein each E is Ser or Ala, or an analogue thereof.

### Embodiment 216

The method of any one of embodiments 201-215, wherein the peptidomimetic macrocycle comprises an amino acid sequence that is at least 60% identical to an amino acid sequence listed in Table 1, Table 1a, Table 1b, Table 1c, Table 2a, or Table 2b.

### Embodiment 217

The method of any one of embodiments 201-216, wherein the peptidomimetic macrocycle comprises an amino acid sequence that is at least 70% identical to an amino acid sequence listed in Table 1, Table 1a, Table 1b, Table 1c, Table 2a, or Table 2b.

### Embodiment 218

The method of any one of embodiments 201-217, wherein the peptidomimetic macrocycle comprises an amino acid sequence that is at least 80% identical to an amino acid sequence listed in Table 1, Table 1a, Table 1b, Table 1c, Table 2a, or Table 2b.

### Embodiment 219

The method of any one of embodiments 201-218, wherein the peptidomimetic macrocycle is at least 60% identical to SP-153, SP-303, SP-331, or SP-671.

### Embodiment 220

The method of any one of embodiments 201-219, wherein the condition is cancer.

### Embodiment 221

The method of any one of embodiments 201-220, wherein the cancer is lymphoma.

### Embodiment 222

The method of any one of embodiments 201-220, wherein the cancer is breast cancer.

### Embodiment 223

The method of any one of embodiments 201-220, wherein the cancer is skin cancer.

### Embodiment 224

The method of any one of embodiments 201-220, wherein the cancer is leukemia.

### Embodiment 225

The method of any one of embodiments 201-220, wherein the cancer is melanoma.

### Embodiment 226

The method of any one of embodiments 201-220, wherein the cancer is bone cancer

### Embodiment 227

The method of any one of embodiments 201-226, wherein the at least one pharmaceutically-active agent, pharmaceutically-acceptable salt, or conjugate thereof is a cyclin-dependent kinase (CDK) inhibitor.

### Embodiment 228

The method of any one of embodiments 201-227, wherein the CDK inhibitor is palbociclib.

### Embodiment 229

The method of any one of embodiments 201-227, wherein the CDK inhibitor is abemaciclib.

### Embodiment 230

The method of any one of embodiments 201-227, wherein the CDK inhibitor is ribociclib.

### Embodiment 231

The method of any one of embodiments 201-226, wherein the at least one pharmaceutically-active agent is a mitogen-activated protein kinase (MEK) inhibitor.

### Embodiment 232

The method of any one of embodiments 201-226, wherein the at least one pharmaceutically-active agent is a microtubule inhibitor.

### Embodiment 233

The method of any one of embodiments 201-226 or 232, wherein the microtubule inhibitor is eribulin.

### Embodiment 234

The method of any one of embodiments 201-226 or 232, wherein the microtubule inhibitor is paclitaxel.

### Embodiment 235

The method of any one of embodiments 201-226, 232, or 234, wherein the microtubule inhibitor is nanoparticle albumin-bound paclitaxel.

What is claimed is:

1. A method of treating a condition in a subject in need thereof, the method comprising administering to the subject a therapeutically-effective amount of a peptidomimetic macrocycle and at least one pharmaceutically-active agent, wherein the peptidomimetic macrocycle and the at least one pharmaceutically-active agent are administered with a time separation of more than 61 minutes.

2. The method of claim 1, wherein the peptidomimetic macrocycle is of the formula: 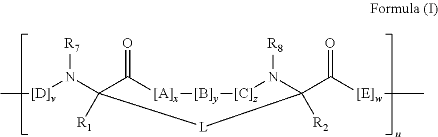
or pharmaceutically acceptable salt thereof, wherein:
each A, C, D, and E is independently an amino acid;
each B is independently an amino acid, 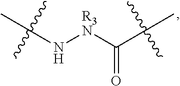
[—NH-L₃-CO—], [—NH-L₃-SO₂—], or [—NH-L₃-];
each R₁ and R₂ is independently hydrogen, alkyl, alkenyl, alkynyl, arylalkyl, cycloalkyl, cycloalkylalkyl, heteroalkyl, or heterocycloalkyl, unsubstituted or substituted with halo-; or
forms a macrocycle-forming linker L′ connected to the alpha position of one of said D or E amino acids;
each R₃ is independently hydrogen, alkyl, alkenyl, alkynyl, arylalkyl, heteroalkyl, cycloalkyl, heterocycloalkyl, cycloalkylalkyl, aryl, or heteroaryl, optionally substituted with R₅;
each L and L′ is independently a macrocycle-forming linker of the formula -L₁-L₂-;
each L₁, L₂, and L₃ is independently alkylene, alkenylene, alkynylene, heteroalkylene, cycloalkylene, heterocycloalkylene, arylene, heteroarylene, or [—R₄—K—R₄-]ₙ, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently hydrogen, alkyl, alkenyl, alkynyl, arylalkyl, cycloalkylalkyl, heterocycloalkyl, a fluorescent moiety, a radioisotope or a therapeutic agent;
each R₇ is independently hydrogen, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
each R₈ is independently hydrogen, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
each v is independently an integer from 1-1000;
each w is independently an integer from 1-1000;
u is 1, 2, 3, 4, 5, 6, 7, 8, 9, or 10;
each x, y and z is independently 1, 2, 3, 4, 5, 6, 7, 8, 9, or 10; and
each n is independently 1, 2, 3, 4, or 5.

3. The method of claim 2, wherein v is 3-10.

4. The method of claim 3, wherein v is 3.

5. The method of claim 2, wherein w is 3-10.

6. The method of claim 5, wherein w is 6.

7. The method of claim 2, wherein x+y+z=6.

8. The method of claim 2, wherein each L₁ and L₂ is independently alkylene, alkenylene, alkynylene, heteroalkylene, cycloalkylene, heterocycloalkylene, arylene, or heteroarylene.

9. The method of claim 8, wherein each L₁ and L₂ is independently alkylene or alkenylene.

10. The method of claim 2, wherein each R₁ and R₂ is independently hydrogen, alkyl, alkenyl, alkynyl, arylalkyl, cycloalkyl, cycloalkylalkyl, heteroalkyl, or heterocycloalkyl, unsubstituted or substituted with halo-.

11. The method of claim 10, wherein each R₁ and R₂ is independently hydrogen.

12. The method of claim 10, wherein each R₁ and R₂ is independently alkyl.

13. The method of claim 10, wherein each R₁ and R₂ is independently methyl.

14. The method of claim 2, wherein u is 1.

15. The method of claim 2, wherein each E is Ser or Ala, or an analogue thereof.

16. The method of claim 1, wherein the peptidomimetic macrocycle comprises an amino acid sequence that is at least 60% identical to an amino acid sequence listed in Table 1, Table 1a, Table 1b, Table 1c, Table 2a, or Table 2b.

17. The method of claim 16, wherein the peptidomimetic macrocycle comprises an amino acid sequence that is at least 70% identical to an amino acid sequence listed in Table 1, Table 1a, Table 1b, Table 1c, Table 2a, or Table 2b.

18. The method of claim 17, wherein the peptidomimetic macrocycle comprises an amino acid sequence that is at least 80% identical to an amino acid sequence listed in Table 1, Table 1a, Table 1b, Table 1c, Table 2a, or Table 2b.

19. The method of claim 16, wherein the peptidomimetic macrocycle is at least 60% identical to SP-153, SP-303, SP-331, or SP-671.

20. The method of claim 1, wherein the condition is cancer.

21. The method of claim 20, wherein the cancer is lymphoma.

22. The method of claim 20, wherein the cancer is breast cancer.

23. The method of claim 20, wherein the cancer is skin cancer.

24. The method of claim 20, wherein the cancer is leukemia.

25. The method of claim 20, wherein the cancer is melanoma.

26. The method of claim 20, wherein the cancer is bone cancer.

27. The method of claim 1, wherein the at least one pharmaceutically-active agent, pharmaceutically-acceptable salt, or conjugate thereof is a cyclin-dependent kinase (CDK) inhibitor.

28. The method of claim 27, wherein the CDK inhibitor is palbociclib.

29. The method of claim 27, wherein the CDK inhibitor is abemaciclib.

30. The method of claim 27, wherein the CDK inhibitor is ribociclib.

31. The method of claim 1, wherein the at least one pharmaceutically-active agent is a mitogen-activated protein kinase (MEK) inhibitor.

32. The method of claim 1, wherein the at least one pharmaceutically-active agent is a microtubule inhibitor.

33. The method of claim 32, wherein the microtubule inhibitor is eribulin.

34. The method of claim 32, wherein the microtubule inhibitor is paclitaxel.

35. The method of claim 34, wherein the microtubule inhibitor is nanoparticle albumin-bound paclitaxel.
